(19) United States
(12) Patent Application Publication
(10) Pub. No.: US 2010/0285144 A1
(43) Pub. Date: Nov. 11, 2010

(54) POLYMER-EPOTHILONE CONJUGATES, PARTICLES, COMPOSITIONS, AND RELATED METHODS OF USE

(76) Inventors: Scott Eliasof, Lexington, MA (US); Thomas C. Crawford, Essex, CT (US); Geeti Gangal, Cambridge, MA (US); Lawrence Alan Reiter, Mystic, CT (US); Pei-Sze Ng, Cambridge, MA (US)

Correspondence Address: LANDO & ANASTASI, LLP, ONE MAIN STREET, SUITE 1100, CAMBRIDGE, MA, 02142, US

(21) Appl. No.: 12/748,818

(22) Filed: Mar. 29, 2010

Related U.S. Application Data
(63) Continuation of application No. PCT/US10/28793, filed on Mar. 26, 2010.
(60) Provisional application No. 61/164,720, filed on Mar. 30, 2009.
(60) Provisional application No. 61/164,722, filed on Mar. 30, 2009.
(60) Provisional application No. 61/164,725, filed on Mar. 30, 2009.
(60) Provisional application No. 61/164,728, filed on Mar. 30, 2009.
(60) Provisional application No. 61/164,731, filed on Mar. 30, 2009.
(60) Provisional application No. 61/164,734, filed on Mar. 30, 2009.
(60) Provisional application No. 61/262,993, filed on Nov. 20, 2009.
(60) Provisional application No. 61/262,994, filed on Nov. 20, 2009.
(60) Provisional application No. 61/281,730, filed on Nov. 20, 2009.
(60) Provisional application No. 61/281,731, filed on Nov. 20, 2009.

(51) Int. Cl. A61K 9/14; A61K 31/765; A61P 35/00; C08G 63/91

(52) U.S. Cl. 424/501; 424/78.38; 424/78.37; 525/411; 525/418

(57) ABSTRACT

Described herein are polymer-agent conjugates and particles, which can be used, for example, in the treatment of cancer or neurological deficits. Also described herein are mixtures, compositions and dosage forms containing the particles, methods of using the particles (e.g., to treat a disorder), kits including the polymer-agent conjugates and particles, methods of making the polymer-agent conjugates and particles, methods of storing the particles and methods of analyzing the particles.

### RELATED APPLICATIONS

This application claims priority to PCT/US 10/28793, filed Mar. 26, 2010; U.S. Ser. No. 61/164,720, filed Mar. 30, 2009; U.S. Ser. No. 61/164,722, filed Mar. 30, 2009; U.S. Ser. No. 61/164,725, filed Mar. 30, 2009; U.S. Ser. No. 61/164,728, filed Mar. 30, 2009; U.S. Ser. No. 61/164,731, filed Mar. 30, 2009; U.S. Ser. No. 61/164,734, filed Mar. 30, 2009; U.S. Ser. No. 61/262,993, filed Nov. 20, 2009; U.S. Ser. No. 61/262,994, filed Nov. 20, 2009; U.S. Ser. No. 61/281,730, filed Nov. 20, 2009; and U.S. Ser. No. 61/281,731, filed Nov. 20, 2009. The disclosures of the prior applications are considered part of (and are incorporated by reference in) the disclosure of this application.

### BACKGROUND OF INVENTION

The delivery of a drug with controlled release of the active agent is desirable to provide optimal use and effectiveness. Controlled release polymer systems may increase the efficacy of the drug and minimize problems with patient compliance.

### SUMMARY OF INVENTION

Described herein are polymer-agent conjugates and particles, which can be used, for example, in the treatment of cancer or neurological deficits. Also described herein are mixtures, compositions and dosage forms containing the particles, methods of using the particles (e.g., to treat a disorder), kits including the polymer-agent conjugates and particles, methods of making the polymer-agent conjugates and particles, methods of storing the particles and methods of analyzing the particles.

Accordingly, in one aspect, the invention features, a polymer-agent conjugate comprising:

a polymer; and

an agent attached to the polymer, wherein the agent is an epothilone.

In some embodiments, the polymer is a biodegradable polymer (e.g., polylactic acid (PLA), polyglycolic acid (PGA), poly(lactic-co-glycolic acid) (PLGA), polycaprolactone (PCL), polydioxanone (PDO), polyanhydrides, polyorthoesters, or chitosan). In some embodiments, the polymer is a hydrophobic polymer. In some embodiments, the polymer is PLA. In some embodiments, the polymer is PGA.

In some embodiments, the polymer is a copolymer of lactic and glycolic acid (e.g., PLGA). In some embodiments, the polymer is a PLGA-ester. In some embodiments, the polymer is a PLGA-lauryl ester. In some embodiments, the polymer comprises a terminal free acid prior to conjugation to an agent. In some embodiments, the polymer comprises a terminal acyl group (e.g., an acetyl group). In some embodiments, the polymer comprises a terminal hydroxyl group. In some embodiments, the ratio of lactic acid monomers to glycolic acid monomers in PLGA is from about 0.1:99.9 to about 99.9:0.1. In some embodiments, the ratio of lactic acid monomers to glycolic acid monomers in PLGA is from about 75:25 to about 25:75, e.g., about 60:40 to about 40:60 (e.g., about 50:50), about 60:40, or about 75:25.

In some embodiments, the weight average molecular weight of the polymer is from about 1 kDa to about 20 kDa (e.g., from about 1 kDa to about 15 kDa, from about 2 kDa to about 12 kDa, from about 6 kDa to about 20 kDa, from about 5 kDa to about 15 kDa, from about 7 kDa to about 11 kDa, from about 5 kDa to about 10 kDa, from about 7 kDa to about 10 kDa, from about 5 kDa to about 7 kDa, from about 6 kDa to about 8 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa or about 17 kDa). In some embodiments, the polymer has a glass transition temperature of about 20° C. to about 60° C. In some embodiments, the polymer has a polymer polydispersity index of less than or equal to about 2.5 (e.g., less than or equal to about 2.2, or less than or equal to about 2.0). In some embodiments, the polymer has a polymer polydispersity index of about 1.0 to about 2.5, e.g., from about 1.0 to about 2.0, from about 1.0 to about 1.8, from about 1.0 to about 1.7, or from about 1.0 to about 1.6.

In some embodiments, the polymer has a hydrophilic portion and a hydrophobic portion. In some embodiments, the polymer is a block copolymer. In some embodiments, the polymer comprises two regions, the two regions together being at least about 70% by weight of the polymer (e.g., at least about 80%, at least about 90%, at least about 95%). In some embodiments, the polymer is a block copolymer comprising a hydrophobic polymer and a hydrophilic polymer. In some embodiments, the polymer, e.g., a diblock copolymer, comprises a hydrophobic polymer and a hydrophilic polymer. In some embodiments, the polymer, e.g., a triblock copolymer, comprises a hydrophobic polymer, a hydrophilic polymer and a hydrophobic polymer, e.g., PLA-PEG-PLA, PGA-PEG-PGA, PLGA-PEG-PLGA, PCL-PEG-PCL, PDO-PEG-PDO, PEG-PLGA-PEG, PLA-PEG-PGA, PGA-PEG-PLA, PLGA-PEG-PLA or PGA-PEG-PLGA.

In some embodiments, the hydrophobic portion of the polymer is a biodegradable polymer (e.g., PLA, PGA, PLGA, PCL, PDO, polyanhydrides, polyorthoesters, or chitosan). In some embodiments, the hydrophobic portion of the polymer is PLA. In some embodiments, the hydrophobic portion of the polymer is PGA. In some embodiments, the hydrophobic portion of the polymer is a copolymer of lactic and glycolic acid (e.g., PLGA). In some embodiments, the hydrophobic portion of the polymer has a weight average molecular weight of from about 1 kDa to about 20 kDa (e.g., from about 1 kDa to about 18 kDa, 17 kDa, 16 kDa, 15 kDa, 14 kDa or 13 kDa, from about 2 kDa to about 12 kDa, from about 6 kDa to about 20 kDa, from about 5 kDa to about 18 kDa, from about 7 kDa to about 17 kDa, from about 8 kDa to about 13 kDa, from about 9 kDa to about 11 kDa, from about 10 kDa to about 14 kDa, from about 6 kDa to about 8 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa or about 17 kDa).

In some embodiments, the hydrophilic portion of the polymer is polyethylene glycol (PEG). In some embodiments, the hydrophilic portion of the polymer has a weight average molecular weight of from about 1 kDa to about 21 kDa (e.g., from about 1 kDa to about 3 kDa, e.g., about 2 kDa, or from about 2 kDa to about 5 kDa, e.g., about 3.5 kDa, or from about 4 kDa to about 6 kDa, e.g., about 5 kDa). In some embodiments, the ratio of the weight average molecular weights of the hydrophilic to hydrophobic portions of the polymer is from about 1:1 to about 1:20 (e.g., about 1:4 to about 1:10, about 1:4 to about 1:7, about 1:3 to about 1:7, about 1:3 to about 1:6, about 1:4 to about 1:6.5 (e.g., 1:4, 1:4.5, 1:5, 1:5.5, 1:6, 1:6.5) or about 1:1 to about 1:4 (e.g., about 1:1.4, 1:1.8, 1:2, 1:2.4, 1:2.8, 1:3, 1:3.2, 1:3.5 or 1:4). In one embodiment, the hydrophilic portion of the polymer has a weight average molecular weight of from about 2 kDa to 3.5 kDa and the ratio of the weight average molecular weight of the hydrophilic to hydrophobic portions of the polymer is from about 1:4 to about 1:6.5 (e.g., 1:4, 1:4.5, 1:5, 1:5.5, 1:6, 1:6.5). In one embodiment, the hydrophilic portion of the polymer has a weight average molecular weight of from about 4 kDa to 6 kDa (e.g., 5 kDa) and the ratio of the weight average molecular weight of the hydrophilic to hydrophobic portions of the polymer is from about 1:1 to about 1:3.5 (e.g., about 1:1.4, 1:1.8, 1:2, 1:2.4, 1:2.8, 1:3, 1:3.2, or 1:3.5).

In some embodiments, the hydrophilic portion of the polymer has a terminal hydroxyl moiety prior to conjugation to an agent. In some embodiments, the hydrophilic portion of has a terminal alkoxy moiety. In some embodiments, the hydrophilic portion of the polymer is a methoxy PEG (e.g., a terminal methoxy PEG). In some embodiments, the hydrophilic polymer portion of the polymer does not have a terminal alkoxy moiety. In some embodiments, the terminus of the hydrophilic polymer portion of the polymer is conjugated to a hydrophobic polymer, e.g., to make a triblock copolymer.

In some embodiments, the hydrophilic portion of the polymer is attached to the hydrophobic portion through a covalent bond. In some embodiments, the hydrophilic polymer is attached to the hydrophobic polymer through an amide, ester, ether, amino, carbamate, or carbonate bond (e.g., an ester or an amide).

In some embodiments, a single agent is attached to a single polymer, e.g., to a terminal end of the polymer. In some embodiments, a plurality of agents are attached to a single polymer (e.g., 2, 3, 4, 5, 6, or more). In some embodiments, the agents are the same agent. In some embodiments, the agents are different agents.

In some embodiments, the agent is an epothilone selected from ixabepilone, epothilone B, epothilone D, BMS310705, dehydelone and ZK-EPO. In some embodiments, the agent is an epothilone described herein.

In some embodiments, the agent is an epothilone attached to the polymer via the hydroxyl group at the 3 position. In some embodiments, the agent is an epothilone attached to the polymer via the hydroxyl group at the 7 position. In some embodiments, the agent is attached directly to the polymer, e.g., through a covalent bond. In some embodiments, the agent is attached to a terminal end of the polymer via an amide, ester, ether, amino, carbamate or carbonate bond. In some embodiments, the agent is attached to a terminal end of the polymer. In some embodiments, the polymer comprises one or more side chains and the agent is directly attached to the polymer through one or more of the side chains.

In some embodiments, the polymer-agent conjugate is:

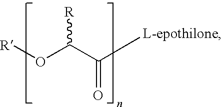

wherein L is a bond or linker, e.g., a linker described herein; and

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the epothilone is selected from ixabepilone, epothilone B, epothilone D, BMS310705, dehydelone and ZK-EPO. In some embodiments, the agent is an epothilone described herein.

In some embodiments, L is a bond.

In some embodiments, L is a linker, e.g., a linker described herein.

In some embodiments, the linker is an alkanoate linker. In some embodiments, the linker is a PEG-based linker. In some embodiments, the linker comprises a disulfide bond. In some embodiments, the linker is a self-immolative linker. In some embodiments, the linker is an amino acid or a peptide (e.g., glutamic acid such as L-glutamic acid, D-glutamic acid, DL-glutamic acid or β-glutamic acid, branched glutamic acid or polyglutamic acid). In some embodiments, the linker is β-alanine glycolate.

In some embodiments, the polymer-agent conjugate is:

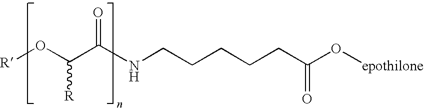

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the epothilone is selected from ixabepilone, epothilone B, epothilone D, BMS310705, dehydelone and ZK-EPO. In some embodiments, the agent is an epothilone described herein.

In some embodiments, the polymer-agent conjugate is:

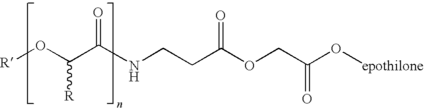

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the epothilone is selected from ixabepilone, epothilone B, epothilone D, BMS310705, dehydelone and ZK-EPO. In some embodiments, the agent is an epothilone described herein.

In some embodiments the linker is a multifunctional linker. In some embodiments, the multifunctional linker has 2, 3, 4, 5, 6 or more reactive moieties that may be functionalized with an agent. In some embodiments, all reactive moieties are functionalized with an agent. In some embodiments, not all of the reactive moieties are functionalized with an agent (e.g., the multifunctional linker has two reactive moieties, and only one reacts with an agent; or the multifunctional linker has four reactive moieties, and only one, two or three react with an agent.)

In some embodiments, two agents are attached to a polymer via a multifunctional linker. In some embodiments, the two agents are the same agent. In some embodiments, the two agents are different agents. In some embodiments, the agent is covalently attached to the polymer via a glutamate linker.

In some embodiments, the polymer-agent conjugate is:

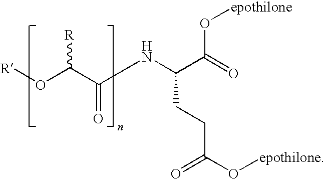

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, each epothilone is independently selected from ixabepilone, epothilone B, epothilone D, BMS310705, dehydelone and ZK-EPO. In some embodiments, each epothilone is independently selected from the epothilones described herein.

In some embodiments, at least one epothilone is attached to the polymer via the hydroxyl group at the 3 position. In some embodiments, at least one epothilone is attached to the polymer via the hydroxyl group at the 7 position. In some embodiments, each epothilone is attached via the same hydroxyl group, e.g., the hydroxyl group at the 3 position or the hydroxyl group at the 7 position. In some embodiments, each epothilone is attached via the hydroxyl group at the 3 position. In some embodiments, each epothilone is attached via the hydroxyl group at the 7 position. In some embodiments, the epothilone molecules may be attached via different hydroxyl groups, e.g., one epothilone is attached via the hydroxyl group at the 3 position and the other epothilone is attached via the hydroxyl group at the 7 position.

In some embodiments, four agents are attached to a polymer via a multifunctional linker. In some embodiments, the four agents are the same agent. In some embodiments, the four agents are different agents. In some embodiments, the agent is covalently attached to the polymer via a tri(glutamate) linker.

In some embodiments, the polymer-agent conjugate is:

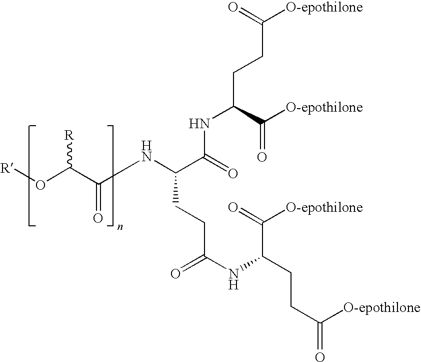

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, each epothilone is independently selected from ixabepilone, epothilone B, epothilone D, BMS310705, dehydelone and ZK-EPO. In some embodiments, each epothilone is independently selected from the epothilones described herein.

In some embodiments, at least one epothilone is attached to the polymer via the hydroxyl group at the 3 position. In some embodiments, at least one epothilone is attached to the polymer via the hydroxyl group at the 7 position. In some embodiments, each epothilone is attached via the same hydroxyl group, e.g., the hydroxyl group at the 3 position or the hydroxyl group at the 7 position. In some embodiments, each epothilone is attached via the hydroxyl group at the 3 position. In some embodiments, each epothilone is attached via the hydroxyl group at the 7 position. In some embodiments, the epothilone molecules may be attached via different hydroxyl groups, e.g., three epothilones are attached via the hydroxyl group at the 3 position and the other epothilone is attached via the hydroxyl group at the 7 position.

In another aspect, the invention features a composition comprising a plurality of polymer-agent conjugates, wherein the polymer-agent conjugate has the following formula:

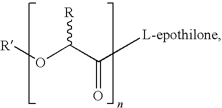

wherein L is a bond or linker, e.g., a linker described herein;

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the epothilone is selected from ixabepilone, epothilone B, epothilone D, BMS310705, dehydelone and ZK-EPO. In some embodiments, the agent is an epothilone described herein.

In some embodiments, L is a bond.

In some embodiments, L is a linker, e.g., a linker described herein.

In some embodiments, the composition comprises a plurality of polymer-agent conjugates wherein the polymer-agent conjugates have the same polymer and the same agent, and differ in the nature of the linkage between the agent and the polymer. For example, in some embodiments, the polymer is PLGA, and the plurality of polymer-agent conjugates includes PLGA polymers attached to an epothilone via the hydroxyl group at the 3 position and PLGA polymers attached to an epothilone via the hydroxyl group at the 7 position.

In another aspect, the invention features a particle. The particle comprises:

a first polymer,

a second polymer having a hydrophilic portion and a hydrophobic portion,

an agent attached to the first polymer or second polymer, wherein the agent is an epothilone, and

optionally, the particle comprises one or more of the following properties:

it further comprises a compound comprising at least one acidic moiety,

wherein the compound is a polymer or a small molecule;

it further comprises a surfactant;

the first polymer is a PLGA polymer, wherein the ratio of lactic acid to glycolic acid is from about 25:75 to about 75:25 and, optionally, the agent is attached to the first polymer;

the first polymer is PLGA polymer, and the weight average molecular weight of the first polymer is from about 1 to about 20 kDa, e.g., is about 1, 2, 3, 4, 5, 6, 7, 8, 9, 10, 11, 12, 13, 14, 15, 16, 17, 18, 19 or 20 kDa; or

the ratio of the first polymer to the second polymer is such that the particle comprises at least 5%, 8%, 10%, 12%, 15%, 18%, 20%, 23%, 25% or 30% by weight of a polymer having a hydrophobic portion and a hydrophilic portion.

In some embodiments, the particle is a nanoparticle. In some embodiments, the nanoparticle has a diameter of less than or equal to about 220 nm (e.g., less than or equal to about 215 nm, 210 nm, 205 nm, 200 nm, 195 nm, 190 nm, 185 nm, 180 nm, 175 nm, 170 nm, 165 nm, 160 nm, 155 nm, 150 nm, 145 nm, 140 nm, 135 nm, 130 nm, 125 nm, 120 nm, 115 nm, 110 nm, 105 nm, 100 nm, 95 nm, 90 nm, 85 nm, 80 nm, 75 nm, 70 nm, 65 nm, 60 nm, 55 nm or 50 nm).

In some embodiments, the particle further comprises a compound comprising at least one acidic moiety, wherein the compound is a polymer or a small molecule.

In some embodiments, the compound comprising at least one acidic moiety is a polymer comprising an acidic group. In some embodiments, the compound comprising at least one acidic moiety is a hydrophobic polymer. In some embodiments, the first polymer and the compound comprising at least one acidic moiety are the same polymer. In some embodiments, the compound comprising at least one acidic moiety is PLGA. In some embodiments, the ratio of lactic acid monomers to glycolic acid monomers in PLGA is from about 0.1:99.9 to about 99.9:0.1. In some embodiments, the ratio of lactic acid monomers to glycolic acid monomers in PLGA is from about 75:25 to about 25:75, e.g., about 60:40 to about 40:60 (e.g., about 50:50), about 60:40, or about 75:25. In some embodiments, the PLGA comprises a terminal hydroxyl group. In some embodiments, the PLGA comprises a terminal acyl group (e.g., an acetyl group).

In some embodiments, the weight average molecular weight of the compound comprising at least one acidic moiety is from about 1 kDa to about 20 kDa (e.g., from about 1 kDa to about 15 kDa, from about 2 kDa to about 12 kDa, from about 6 kDa to about 20 kDa, from about 5 kDa to about 15 kDa, from about 7 kDa to about 11 kDa, from about 5 kDa to about 10 kDa, from about 7 kDa to about 10 kDa, from about 5 kDa to about 7 kDa, from about 6 kDa to about 8 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa or about 17 kDa). In some embodiments, the compound comprising at least one acidic moiety has a glass transition temperature of from about 20° C. to about 60° C.

In some embodiments, the compound comprising at least one acidic moiety has a polymer polydispersity index of less than or equal to about 2.5 (e.g., less than or equal to about 2.2, or less than or equal to about 2.0). In some embodiments, the compound comprising at least one acidic moiety has a polymer polydispersity index of about 1.0 to about 2.5, e.g., from about 1.0 to about 2.0, from about 1.0 to about 1.8, from about 1.0 to about 1.7, or from about 1.0 to about 1.6.

In some embodiments, the particle comprises a plurality of compounds comprising at least one acidic moiety. For example, in some embodiments, one compound of the plurality of compounds comprising at least one acidic moiety is a PLGA polymer wherein the hydroxy terminus is functionalized with an acetyl group, and another compound in the plurality is a PLGA polymer wherein the hydroxy terminus is unfunctionalized.

In some embodiments, the percent by weight of the compound comprising at least one acidic moiety within the particle is up to about 50% (e.g., up to about 45% by weight, up to about 40% by weight, up to about 35% by weight, up to about 30% by weight, from about 0 to about 30% by weight, e.g., about 4.5%, about 9%, about 12%, about 15%, about 18%, about 20%, about 22%, about 24%, about 26%, about 28% or about 30%).

In some embodiments, the compound comprising at least one acidic moiety is a small molecule comprising an acidic group.

In some embodiments, the particle further comprises a surfactant. In some embodiments, the surfactant is PEG, poly(vinyl alcohol) (PVA), poly(vinylpyrrolidone) (PVP), poloxamer, a polysorbate, a polyoxyethylene ester, a PEG-lipid (e.g., PEG-ceramide, d-alpha-tocopheryl polyethylene glycol 1000 succinate), 1,2-Distearoyl-sn-Glycero-3-[Phospho-rac-(1-glycerol)] or lecithin. In some embodiments, the surfactant is PVA and the PVA is from about 3 kDa to about 50 kDa (e.g., from about 5 kDa to about 45 kDa, about 7 kDa to about 42 kDa, from about 9 kDa to about 30 kDa, or from about 11 to about 28 kDa) and up to about 98% hydrolyzed (e.g., about 75-95%, about 80-90% hydrolyzed, or about 85% hydrolyzed). In some embodiments, the surfactant is polysorbate 80. In some embodiments, the surfactant is Solutol® HS 15. In some embodiments, the surfactant is present in an amount of up to about 35% by weight of the particle (e.g., up to about 20% by weight or up to about 25% by weight, from about 15% to about 35% by weight, from about 20% to about 30% by weight, or from about 23% to about 26% by weight).

In some embodiments, the particle further comprises a stabilizer or lyoprotectant, e.g., a stabilizer or lyoprotectant described herein. In some embodiments, the stabilizer or lyoprotectant is a carbohydrate (e.g., a carbohydrate described herein, such as, e.g., sucrose, cyclodextrin or a derivative of cyclodextrin (e.g. 2-hydroxypropyl-β-cyclodextrin)), salt, PEG, PVP or crown ether.

In some embodiments, the agent is attached to the first polymer to form a polymer-agent conjugate. In some embodiments, the agent is attached to the second polymer to form a polymer-agent conjugate.

In some embodiments the amount of agent in the particle that is not attached to the first or second polymer is less than about 5% (e.g., less than about 2% or less than about 1%, e.g., in terms of w/w or number/number) of the amount of agent attached to the first polymer or second polymer.

In some embodiments, the first polymer is a biodegradable polymer (e.g., PLA, PGA, PLGA, PCL, PDO, polyanhydrides, polyorthoesters, or chitosan). In some embodiments, the first polymer is a hydrophobic polymer. In some embodiments, the percent by weight of the first polymer within the particle is from about 20% to about 90% (e.g., from about 20% to about 80%, from about 25% to about 75%, or from about 30% to about 70%). In some embodiments, the first polymer is PLA. In some embodiments, the first polymer is PGA.

In some embodiments, the first polymer is a copolymer of lactic and glycolic acid (e.g., PLGA). In some embodiments, the first polymer is a PLGA-ester. In some embodiments, the first polymer is a PLGA-lauryl ester. In some embodiments, the first polymer comprises a terminal free acid. In some embodiments, the first polymer comprises a terminal acyl group (e.g., an acetyl group). In some embodiments, the polymer comprises a terminal hydroxyl group. In some embodiments, the ratio of lactic acid monomers to glycolic acid monomers in PLGA is from about 0.1:99.9 to about 99.9:0.1. In some embodiments, the ratio of lactic acid monomers to glycolic acid monomers in PLGA is from about 75:25 to about 25:75, e.g., about 60:40 to about 40:60 (e.g., about 50:50), about 60:40, or about 75:25.

In some embodiments, the weight average molecular weight of the first polymer is from about 1 kDa to about 20 kDa (e.g., from about 1 kDa to about 15 kDa, from about 2 kDa to about 12 kDa, from about 6 kDa to about 20 kDa, from about 5 kDa to about 15 kDa, from about 7 kDa to about 11 kDa, from about 5 kDa to about 10 kDa, from about 7 kDa to about 10 kDa, from about 5 kDa to about 7 kDa, from about 6 kDa to about 8 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa or about 17 kDa). In some embodiments, the first polymer has a glass transition temperature of from about 20° C. to about 60° C. In some embodiments, the first polymer has a polymer polydispersity index of less than or equal to about 2.5 (e.g., less than or equal to about 2.2, or less than or equal to about 2.0). In some embodiments, the first polymer has a polymer polydispersity index of about 1.0 to about 2.5, e.g., from about 1.0 to about 2.0, from about 1.0 to about 1.8, from about 1.0 to about 1.7, or from about 1.0 to about 1.6.

In some embodiments, the percent by weight of the second polymer within the particle is up to about 50% by weight (e.g., from about 4 to any of about 50%, about 5%, about 8%, about 10%, about 15%, about 20%, about 23%, about 25%, about 30%, about 35%, about 40%, about 45% or about 50% by weight). For example, the percent by weight of the second polymer within the particle is from about 3% to 30%, from about 5% to 25% or from about 8% to 23%. In some embodiments, the second polymer has a hydrophilic portion and a hydrophobic portion. In some embodiments, the second polymer is a copolymer, e.g., a block copolymer. In some embodiments, the second polymer comprises two regions, the two regions together being at least about 70% by weight of the polymer (e.g., at least about 80%, at least about 90%, at least about 95%). In some embodiments, the second polymer is a block copolymer comprising a hydrophobic polymer and a hydrophilic polymer. In some embodiments, the second polymer, e.g., a diblock copolymer, comprises a hydrophobic polymer and a hydrophilic polymer. In some embodiments, the second polymer, e.g., a triblock copolymer, comprises a hydrophobic polymer, a hydrophilic polymer and a hydrophobic polymer, e.g., PLA-PEG-PLA, PGA-PEG-PGA, PLGA-PEG-PLGA, PCL-PEG-PCL, PDO-PEG-PDO, PEG-PLGA-PEG, PLA-PEG-PGA, PGA-PEG-PLA, PLGA-PEG-PLA or PGA-PEG-PLGA.

In some embodiments, the hydrophobic portion of the second polymer is a biodegradable polymer (e.g., PLA, PGA, PLGA, PCL, PDO, polyanhydrides, polyorthoesters, or chitosan). In some embodiments, the hydrophobic portion of the second polymer is PLA. In some embodiments, the hydrophobic portion of the second polymer is PGA. In some embodiments, the hydrophobic portion of the second polymer is a copolymer of lactic and glycolic acid (e.g., PLGA). In some embodiments, the hydrophobic portion of the second polymer has a weight average molecular weight of from about 1 kDa to about 20 kDa (e.g., from about 1 kDa to about 18 kDa, 17 kDa, 16 kDa, 15 kDa, 14 kDa or 13 kDa, from about 2 kDa to about 12 kDa, from about 6 kDa to about 20 kDa, from about 5 kDa to about 18 kDa, from about 7 kDa to about 17 kDa, from about 8 kDa to about 13 kDa, from about 9 kDa to about 11 kDa, from about 10 kDa to about 14 kDa, from about 6 kDa to about 8 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa or about 17 kDa).

In some embodiments, the hydrophilic polymer portion of the second polymer is PEG. In some embodiments, the hydrophilic portion of the second polymer has a weight average molecular weight of from about 1 kDa to about 21 kDa (e.g., from about 1 kDa to about 3 kDa, e.g., about 2 kDa, or from about 2 kDa to about 5 kDa, e.g., about 3.5 kDa, or from about 4 kDa to about 6 kDa, e.g., about 5 kDa). In some embodiments, the ratio of weight average molecular weight of the hydrophilic to hydrophobic polymer portions of the second polymer from about 1:1 to about 1:20 (e.g., about 1:4 to about 1:10, about 1:4 to about 1:7, about 1:3 to about 1:7, about 1:3 to about 1:6, about 1:4 to about 1:6.5 (e.g., 1:4, 1:4.5, 1:5, 1:5.5, 1:6, 1:6.5) or about 1:1 to about 1:4 (e.g., about 1:1.4, 1:1.8, 1:2, 1:2.4, 1:2.8, 1:3, 1:3.2, 1:3.5 or 1:4). In one embodiment, the hydrophilic portion of the second polymer has a weight average molecular weight of from about 2 kDa to 3.5 kDa and the ratio of the weight average molecular weight of the hydrophilic to hydrophobic portions of the second polymer is from about 1:4 to about 1:6.5 (e.g., 1:4, 1:4.5, 1:5, 1:5.5, 1:6, 1:6.5). In one embodiment, the hydrophilic portion of the second polymer has a weight average molecular weight of from about 4 kDa to 6 kDa (e.g., 5 kDa) and the ratio of the weight average molecular weight of the hydrophilic to hydrophobic portions of the second polymer is from about 1:1 to about 1:3.5 (e.g., about 1:1.4, 1:1.8, 1:2, 1:2.4, 1:2.8, 1:3, 1:3.2, or 1:3.5).

In some embodiments, the hydrophilic polymer portion of the second polymer has a terminal hydroxyl moiety. In some embodiments, the hydrophilic polymer portion of the second polymer has a terminal alkoxy moiety. In some embodiments, the hydrophilic polymer portion of the second polymer is a methoxy PEG (e.g., a terminal methoxy PEG). In some embodiments, the hydrophilic polymer portion of the second polymer does not have a terminal alkoxy moiety. In some embodiments, the terminus of the hydrophilic polymer portion of the second polymer is conjugated to a hydrophobic polymer, e.g., to make a triblock copolymer.

In some embodiments, the hydrophilic polymer portion of the second polymer comprises a terminal conjugate. In some embodiments, the terminal conjugate is a targeting agent or a dye. In some embodiments, the terminal conjugate is a folate or a rhodamine. In some embodiments, the terminal conjugate is a targeting peptide (e.g., an RGD peptide).

In some embodiments, the hydrophilic polymer portion of the second polymer is attached to the hydrophobic polymer portion through a covalent bond. In some embodiments, the hydrophilic polymer is attached to the hydrophobic polymer through an amide, ester, ether, amino, carbamate, or carbonate bond (e.g., an ester or an amide).

In some embodiments, the ratio by weight of the first to the second polymer is from about 1:1 to about 20:1, e.g., about 1:1 to about 10:1, e.g., about 1:1 to 9:1, or about 1.2: to 8:1. In some embodiments, the ratio of the first and second polymer is from about 85:15 to about 55:45 percent by weight or about 84:16 to about 60:40 percent by weight. In some embodiments, the ratio by weight of the first polymer to the compound comprising at least one acidic moiety is from about 1:3 to about 1000:1, e.g., about 1:1 to about 10:1, or about 1.5:1. In some embodiments, the ratio by weight of the second polymer to the compound comprising at least one acidic moiety is from about 1:10 to about 250:1, e.g., from about 1:5 to about 5:1, or from about 1:3.5 to about 1:1.

In some embodiments the particle is substantially free of a targeting agent (e.g., of a targeting agent covalently linked to a component of the particle, e.g., to the first or second polymer or agent), e.g., a targeting agent able to bind to or otherwise associate with a target biological entity, e.g., a membrane component, a cell surface receptor, prostate specific membrane antigen, or the like. In some embodiments the particle is substantially free of a targeting agent that causes the particle to become localized to a tumor, a disease site, a tissue, an organ, a type of cell, e.g., a cancer cell, within the body of a subject to whom a therapeutically effective amount of the particle is administered. In some embodiments, the particle is substantially free of a targeting agent selected from nucleic acid aptamers, growth factors, hormones, cytokines, interleukins, antibodies, integrins, fibronectin receptors, p-glycoprotein receptors, peptides and cell binding sequences. In some embodiments, no polymer is conjugated to a targeting moiety. In an embodiment substantially free of a targeting agent means substantially free of any moiety other than the first polymer, the second polymer, a third polymer (if present), a surfactant (if present), and the agent, e.g., an epothilone or anti-cancer agent, that targets the particle. Thus, in such embodiments, any contribution to localization by the first polymer, the second polymer, a third polymer (if present), a surfactant (if present), and the agent is not considered to be “targeting.” In an embodiment the particle is free of moieties added for the purpose of selectively targeting the particle to a site in a subject, e.g., by the use of a moiety on the particle having a high and specific affinity for a target in the subject.

In some embodiments the second polymer is other than a lipid, e.g., other than a phospholipid. In some embodiments the particle is substantially free of an amphiphilic layer that reduces water penetration into the nanoparticle. In some embodiment the particle comprises less than 5 or 10% (e.g., as determined as w/w, v/v) of a lipid, e.g., a phospholipid. In some embodiments the particle is substantially free of a lipid layer, e.g., a phospholipid layer, e.g., that reduces water penetration into the nanoparticle. In some embodiments the particle is substantially free of lipid, e.g., is substantially free of phospholipid.

In some embodiments the agent is covalently bound to a PLGA polymer.

In some embodiments the particle is substantially free of a radiopharmaceutical agent, e.g., a radiotherapeutic agent, radiodiagnostic agent, prophylactic agent, or other radioisotope. In some embodiments the particle is substantially free of an immunomodulatory agent, e.g., an immunostimulatory agent or immunosuppressive agent. In some embodiments the particle is substantially free of a vaccine or immunogen, e.g., a peptide, sugar, lipid-based immunogen, B cell antigen or T cell antigen. In some embodiments, the particle is substantially free of water soluble PLGA (e.g., PLGA having a weight average molecular weight of less than about 1 kDa).

In some embodiments, the ratio of the first polymer to the second polymer is such that the particle comprises at least 5%, 8%, 10%, 12%, 15%, 18%, 20%, 23%, 25%, or 30% by weight of a polymer having a hydrophobic portion and a hydrophilic portion.

In some embodiments, the zeta potential of the particle surface, when measured in water, is from about −80 mV to about 50 mV, e.g., about −50 mV to about 30 mV, about −20 mV to about 20 mV, or about −10 mV to about 10 mV. In some embodiments, the zeta potential of the particle surface, when measured in water, is neutral or slightly negative. In some embodiments, the zeta potential of the particle surface, when measured in water, is less than 0, e.g., about 0 mV to about −20 mV.

In some embodiments, the particle comprises less than 5000 ppm of a solvent (e.g., acetone, tert-butylmethyl ether, heptane, dichloromethane, dimethylformamide, ethyl acetate, acetonitrile, tetrahydrofuran, ethanol, methanol, isopropyl alcohol, methyl ethyl ketone, butyl acetate, or propyl acetate), (e.g., less than 4500 ppm, less than 4000 ppm, less than 3500 ppm, less than 3000 ppm, less than 2500 ppm, less than 2000 ppm, less than 1500 ppm, less than 1000 ppm, less than 500 ppm, less than 250 ppm, less than 100 ppm, less than 50 ppm, less than 25 ppm, less than 10 ppm, less than 5 ppm, less than 2 ppm, or less than 1 ppm). In some embodiments, the particle is substantially free of a solvent (e.g., acetone, tert-butylmethyl ether, heptane, dichloromethane, dimethylformamide, ethyl acetate, acetonitrile, tetrahydrofuran, ethanol, methanol, isopropyl alcohol, methyl ethyl ketone, butyl acetate, or propyl acetate).

In some embodiments, the particle is substantially free of a class II or class III solvent as defined by the United States Department of Health and Human Services Food and Drug Administration “Q3c-Tables and List.” In some embodiments, the particle comprises less than 5000 ppm of acetone. In some embodiments, the particle comprises less than 5000 ppm of tert-butylmethyl ether. In some embodiments, the particle comprises less than 5000 ppm of heptane. In some embodiments, the particle comprises less than 600 ppm of dichloromethane. In some embodiments, the particle comprises less than 880 ppm of dimethylformamide. In some embodiments, the particle comprises less than 5000 ppm of ethyl acetate. In some embodiments, the particle comprises less than 410 ppm of acetonitrile. In some embodiments, the particle comprises less than 720 ppm of tetrahydrofuran. In some embodiments, the particle comprises less than 5000 ppm of ethanol. In some embodiments, the particle comprises less than 3000 ppm of methanol. In some embodiments, the particle comprises less than 5000 ppm of isopropyl alcohol. In some embodiments, the particle comprises less than 5000 ppm of methyl ethyl ketone. In some embodiments, the particle comprises less than 5000 ppm of butyl acetate. In some embodiments, the particle comprises less than 5000 ppm of propyl acetate.

In some embodiments, a composition comprising a plurality of particles is substantially free of solvent.

In some embodiments, in a composition of a plurality of particles, the particles have an average diameter of from about 50 nm to about 500 nm (e.g., from about 50 to about 200 nm). In some embodiments, in a composition of a plurality of particles, the particles have a Dv50 (median particle size) from about 50 nm to about 220 nm (e.g., from about 75 nm to about 200 nm). In some embodiments, in a composition of a plurality of particles, the particles have a Dv90 (particle size below which 90% of the volume of particles exists) of about 50 nm to about 500 nm (e.g., about 75 nm to about 220 nm).

In some embodiments, a single agent is attached to a single polymer (e.g., a single first polymer or a single second polymer), e.g., to a terminal end of the polymer. In some embodiments, a plurality of agents are attached to a single polymer (e.g., a single first polymer or a single second polymer) (e.g., 2, 3, 4, 5, 6, or more). In some embodiments, the agents are the same agent. In some embodiments, the agents are different agents.

In some embodiments, the agent is an epothilone selected from ixabepilone, epothilone B, epothilone D, BMS310705, dehydelone and ZK-EPO. In some embodiments, the agent is an epothilone described herein.

In some embodiments, the agent is an epothilone attached to the polymer via the hydroxyl group at the 3 position. In some embodiments, the agent is an epothilone attached to the polymer via the hydroxyl group at the 7 position.

In some embodiments, the agent is attached directly to the polymer, e.g., through a covalent bond. In some embodiments, the agent is attached to a terminal end of the polymer via an amide, ester, ether, amino, carbamate or carbonate bond. In some embodiments, the agent is attached to a terminal end of the polymer. In some embodiments, the polymer comprises one or more side chains and the agent is directly attached to the polymer through one or more of the side chains.

In some embodiments, the polymer-agent conjugate in the particle, e.g., the nanoparticle, is:

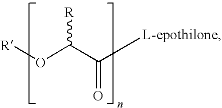

wherein L is a bond or linker, e.g., a linker described herein; and

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the epothilone is selected from ixabepilone, epothilone B, epothilone D, BMS310705, dehydelone and ZK-EPO. In some embodiments, the agent is an epothilone described herein.

In some embodiments, L is a bond.

In some embodiments, L is a linker, e.g., a linker described herein.

In some embodiments, the linker is an alkanoate linker. In some embodiments, the linker is a PEG-based linker. In some embodiments, the linker comprises a disulfide bond. In some embodiments, the linker is a self-immolative linker. In some embodiments, the linker is an amino acid or a peptide (e.g., glutamic acid such as L-glutamic acid, D-glutamic acid, DL-glutamic acid or β-glutamic acid, branched glutamic acid or polyglutamic acid). In some embodiments, the linker is β-alanine glycolate.

In some embodiments, the polymer-agent conjugate in the particle, e.g., the nanoparticle, is:

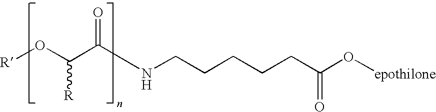

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the epothilone is selected from ixabepilone, epothilone B, epothilone D, BMS310705, dehydelone and ZK-EPO. In some embodiments, the agent is an epothilone described herein.

In some embodiments, the polymer-agent conjugate in the particle, e.g., the nanoparticle, is:

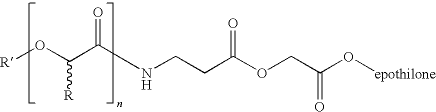

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the epothilone is selected from ixabepilone, epothilone B, epothilone D, BMS310705, dehydelone and ZK-EPO. In some embodiments, the agent is an epothilone described herein.

In some embodiments the linker is a multifunctional linker. In some embodiments, the multifunctional linker has 2, 3, 4, 5, 6 or more reactive moieties that may be functionalized with an agent. In some embodiments, all reactive moieties are functionalized with an agent. In some embodiments, not all of the reactive moieties are functionalized with an agent (e.g., the multifunctional linker has two reactive moieties, and only one reacts with an agent; or the multifunctional linker has four reactive moieties, and only one, two or three react with an agent.)

In some embodiments, two agents are attached to a polymer via a multifunctional linker. In some embodiments, the two agents are the same agent. In some embodiments, the two agents are different agents. In some embodiments, the agent is covalently attached to the polymer via a glutamate linker.

In some embodiments, the polymer-agent conjugate in the particle, e.g., the nanoparticle, is:

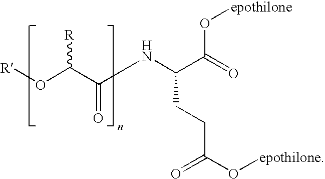

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, each epothilone is independently selected from ixabepilone, epothilone B, epothilone D, BMS310705, dehydelone and ZK-EPO. In some embodiments, each epothilone is independently selected from the epothilones described herein.

In some embodiments, at least one epothilone is attached to the polymer via the hydroxyl group at the 3 position. In some embodiments, at least one epothilone is attached to the polymer via the hydroxyl group at the 7 position. In some embodiments, each epothilone is attached via the same hydroxyl group, e.g., the hydroxyl group at the 3 position or the hydroxyl group at the 7 position. In some embodiments, each epothilone is attached via the hydroxyl group at the 3 position. In some embodiments, each epothilone is attached via the hydroxyl group at the 7 position. In some embodiments, the epothilone molecules may be attached via different hydroxyl groups, e.g., one epothilone is attached via the hydroxyl group at the 3 position and the other epothilone is attached via the hydroxyl group at the 7 position.

In some embodiments, four agents are attached to a polymer via a multifunctional linker. In some embodiments, the four agents are the same agent. In some embodiments, the four agents are different agents. In some embodiments, the agent is covalently attached to the polymer via a tri(glutamate) linker.

In some embodiments, the polymer-agent conjugate in the particle, e.g., the nanoparticle, is:

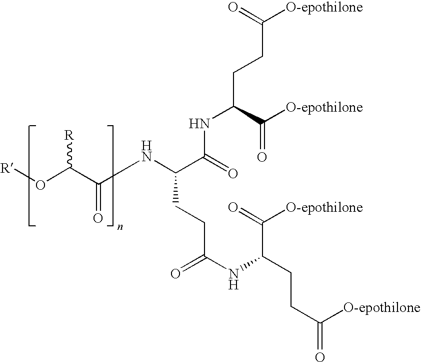

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, each epothilone is independently selected from ixabepilone, epothilone B, epothilone D, BMS310705, dehydelone and ZK-EPO. In some embodiments, each epothilone is independently selected from the epothilones described herein.

In some embodiments, at least one epothilone is attached to the polymer via the hydroxyl group at the 3 position. In some embodiments, at least one epothilone is attached to the polymer via the hydroxyl group at the 7 position. In some embodiments, each epothilone is attached via the same hydroxyl group, e.g., the hydroxyl group at the 3 position or the hydroxyl group at the 7 position. In some embodiments, each epothilone is attached via the hydroxyl group at the 3 position. In some embodiments, each epothilone is attached via the hydroxyl group at the 7 position. In some embodiments, the epothilone molecules may be attached via different hydroxyl groups, e.g., three epothilones are attached via the hydroxyl group at the 3 position and the other epothilone is attached via the hydroxyl group at the 7 position.

In some embodiments, the particle comprises a plurality of polymer-agent conjugates. In some embodiments, the plurality of polymer-agent conjugates have the same polymer and the same agent, and differ in the nature of the linkage between the agent and the polymer. For example, in some embodiments, the polymer is PLGA, and the plurality of polymer-agent conjugates includes PLGA polymers attached to an epothilone via the hydroxyl group at the 3 position, and PLGA polymers attached to an epothilone via the hydroxyl group at the 7 position. In some embodiments, the polymer is PLGA, and the plurality of polymer-agent conjugates includes epothilone molecules attached to more than one polymer chain, e.g., epothilone molecules with PLGA polymers attached to the hydroxyl group at the 3 position and the hydroxyl group at the 7 position.

In some embodiments, the plurality of polymer-agent conjugates have the same polymer and the same agent, but the agent may be attached to the polymer via different linkers. In some embodiments, the plurality of polymer-agent conjugates includes a polymer directly attached to an agent and a polymer attached to an agent via a linker. In an embodiment, one agent is released from one polymer-agent conjugate in the plurality with a first release profile and a second agent is released from a second polymer-agent conjugate in the plurality with a second release profile. E.g., a bond between the first agent and the first polymer is more rapidly broken than a bond between the second agent and the second polymer. E.g., the first polymer-agent conjugate can comprise a first linker linking the first agent to the first polymer and the second polymer-agent conjugate can comprise a second linker linking the second agent to the second polymer, wherein the linkers provide for different profiles for release of the first and second agents from their respective agent-polymer conjugates.

In some embodiments, the plurality of polymer-agent conjugates includes different polymers. In some embodiments, the plurality of polymer-agent conjugates includes different agents.

In some embodiments, the agent is present in the particle in an amount of from about 1 to about 30% by weight (e.g., from about 3 to about 30% by weight, from about 4 to about 25% by weight, or from about 5 to about 13%, 14%, 15%, 16%, 17%, 18%, 19% or 20% by weight).

In an embodiment the particle comprises the enumerated elements.

In an embodiment the particle consists of the enumerated elements.

In an embodiment the particle consists essentially of the enumerated elements.

In another aspect, the invention features a particle. The particle comprises:

a first polymer,

a second polymer having a hydrophilic portion and a hydrophobic portion,

an agent, wherein the agent is an epothilone, and wherein the agent is attached to the first polymer to form a polymer-agent conjugate, and

optionally, the particle comprises one or more of the following:

it further comprises a compound comprising at least one acidic moiety,

wherein the compound is a polymer or a small molecule;

it further comprises a surfactant;

the first polymer is a PLGA polymer, wherein the ratio of lactic acid to glycolic acid is from about 25:75 to about 75:25 and the agent is attached to the first polymer;

the first polymer is PLGA polymer, and the weight average molecular weight of the first polymer is from about 1 to about 20 kDa, e.g., is about 1, 2, 3, 4, 5, 6, 7, 8, 9, 10, 11, 12, 13, 14, 15, 16, 17, 18, 19 or 20 kDa; or

the ratio of the first polymer to the second polymer is such that the particle comprises at least 5%, 8%, 10%, 12%, 15%, 18%, 20%, 23%, 25% or 30% by weight of a polymer having a hydrophobic portion and a hydrophilic portion.

In some embodiments, the particle is a nanoparticle. In some embodiments, the nanoparticle has a diameter of less than or equal to about 220 nm (e.g., less than or equal to about 215 nm, 210 nm, 205 nm, 200 nm, 195 nm, 190 nm, 185 nm, 180 nm, 175 nm, 170 nm, 165 nm, 160 nm, 155 nm, 150 nm, 145 nm, 140 nm, 135 nm, 130 nm, 125 nm, 120 nm, 115 nm, 110 nm, 105 nm, 100 nm, 95 nm, 90 nm, 85 nm, 80 nm, 75 nm, 70 nm, 65 nm, 60 nm, 55 nm or 50 nm).

In some embodiments, the particle further comprises a compound comprising at least one acidic moiety, wherein the compound is a polymer or a small molecule.

In some embodiments, the compound comprising at least one acidic moiety is a polymer comprising an acidic group. In some embodiments, the compound comprising at least one acidic moiety is a hydrophobic polymer. In some embodiments, the first polymer and the compound comprising at least one acidic moiety are the same polymer. In some embodiments, the compound comprising at least one acidic moiety is PLGA. In some embodiments, the ratio of lactic acid monomers to glycolic acid monomers in PLGA is from about 0.1:99.9 to about 99.9:0.1. In some embodiments, the ratio of lactic acid monomers to glycolic acid monomers in PLGA is from about 75:25 to about 25:75, e.g., about 60:40 to about 40:60 (e.g., about 50:50), about 60:40, or about 75:25. In some embodiments, the PLGA comprises a terminal hydroxyl group. In some embodiments, the PLGA comprises a terminal acyl group (e.g., an acetyl group).

In some embodiments, the weight average molecular weight of the compound comprising at least one acidic moiety is from about 1 kDa to about 20 kDa (e.g., from about 1 kDa to about 15 kDa, from about 2 kDa to about 12 kDa, from about 6 kDa to about 20 kDa, from about 5 kDa to about 15 kDa, from about 7 kDa to about 11 kDa, from about 5 kDa to about 10 kDa, from about 7 kDa to about 10 kDa, from about 5 kDa to about 7 kDa, from about 6 kDa to about 8 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa or about 17 kDa). In some embodiments, the compound comprising at least one acidic moiety has a glass transition temperature of from about 20° C. to about 60° C.

In some embodiments, the compound comprising at least one acidic moiety has a polymer polydispersity index of less than or equal to about 2.5 (e.g., less than or equal to about 2.2, or less than or equal to about 2.0). In some embodiments, the compound comprising at least one acidic moiety has a polymer polydispersity index of about 1.0 to about 2.5, e.g., from about 1.0 to about 2.0, from about 1.0 to about 1.8, from about 1.0 to about 1.7, or from about 1.0 to about 1.6.

In some embodiments, the particle comprises a plurality of compounds comprising at least one acidic moiety. For example, in some embodiments, one compound of the plurality of compounds comprising at least one acidic moiety is a PLGA polymer wherein the hydroxy terminus is functionalized with an acetyl group, and another compound in the plurality is a PLGA polymer wherein the hydroxy terminus is unfunctionalized.

In some embodiments, the percent by weight of the compound comprising at least one acidic moiety within the particle is up to about 50% (e.g., up to about 45% by weight, up to about 40% by weight, up to about 35% by weight, up to about 30% by weight, from about 0 to about 30% by weight, e.g., about 4.5%, about 9%, about 12%, about 15%, about 18%, about 20%, about 22%, about 24%, about 26%, about 28%, or about 30%).

In some embodiments, the compound comprising at least one acidic moiety is a small molecule comprising an acidic group.

In some embodiments, the particle further comprises a surfactant. In some embodiments, the surfactant is PEG, PVA, PVP, poloxamer, a polysorbate, a polyoxyethylene ester, a PEG-lipid (e.g., PEG-ceramide, d-alpha-tocopheryl polyethylene glycol 1000 succinate), 1,2-Distearoyl-sn-Glycero-3-[Phospho-rac-(1-glycerol)] or lecithin. In some embodiments, the surfactant is PVA and the PVA is from about 3 kDa to about 50 kDa (e.g., from about 5 kDa to about 45 kDa, about 7 kDa to about 42 kDa, from about 9 kDa to about 30 kDa, or from about 11 to about 28 kDa) and up to about 98% hydrolyzed (e.g., about 75-95%, about 80-90% hydrolyzed, or about 85% hydrolyzed). In some embodiments, the surfactant is polysorbate 80. In some embodiments, the surfactant is Solutol® HS 15. In some embodiments, the surfactant is present in an amount of up to about 35% by weight of the particle (e.g., up to about 20% by weight or up to about 25% by weight, from about 15% to about 35% by weight, from about 20% to about 30% by weight, or from about 23% to about 26% by weight).

In some embodiments, the particle further comprises a stabilizer or lyoprotectant, e.g., a stabilizer or lyoprotectant described herein. In some embodiments, the stabilizer or lyoprotectant is a carbohydrate (e.g., a carbohydrate described herein, such as, e.g., sucrose, cyclodextrin or a derivative of cyclodextrin (e.g. 2-hydroxypropyl-β-cyclodextrin)), salt, PEG, PVP or crown ether.

In an embodiment the amount of agent in the particle that is not attached to the first polymer is less than about 5% (e.g., less than about 2% or less than about 1%, e.g., in terms of w/w or number/number) of the amount of agent attached to the first polymer.

In some embodiments, the first polymer is a biodegradable polymer (e.g., PLA, PGA, PLGA, PCL, PDO, polyanhydrides, polyorthoesters, or chitosan). In some embodiments, the first polymer is a hydrophobic polymer. In some embodiments, the percent by weight of the first polymer within the particle is from about 20% to about 90% (e.g., from about 20% to about 80%, from about 25% to about 75%, or from about 30% to about 70%). In some embodiments, the first polymer is PLA. In some embodiments, the first polymer is PGA.

In some embodiments, the first polymer is a copolymer of lactic and glycolic acid (e.g., PLGA). In some embodiments, the first polymer is a PLGA-ester. In some embodiments, the first polymer is a PLGA-lauryl ester. In some embodiments, the first polymer comprises a terminal free acid. In some embodiments, the first polymer comprises a terminal acyl group (e.g., an acetyl group). In some embodiments, the polymer comprises a terminal hydroxyl group. In some embodiments, the ratio of lactic acid monomers to glycolic acid monomers in PLGA is from about 0.1:99.9 to about 99.9:0.1. In some embodiments, the ratio of lactic acid monomers to glycolic acid monomers in PLGA is from about 75:25 to about 25:75, e.g., about 60:40 to about 40:60 (e.g., about 50:50), about 60:40, or about 75:25.

In some embodiments, the weight average molecular weight of the first polymer is from about 1 kDa to about 20 kDa (e.g., from about 1 kDa to about 15 kDa, from about 2 kDa to about 12 kDa, from about 6 kDa to about 20 kDa, from about 5 kDa to about 15 kDa, from about 7 kDa to about 11 kDa, from about 5 kDa to about 10 kDa, from about 7 kDa to about 10 kDa, from about 5 kDa to about 7 kDa, from about 6 kDa to about 8 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa or about 17 kDa). In some embodiments, the first polymer has a glass transition temperature of from about 20° C. to about 60° C. In some embodiments, the first polymer has a polymer polydispersity index of less than or equal to about 2.5 (e.g., less than or equal to about 2.2, or less than or equal to about 2.0). In some embodiments, the first polymer has a polymer polydispersity index of about 1.0 to about 2.5, e.g., from about 1.0 to about 2.0, from about 1.0 to about 1.8, from about 1.0 to about 1.7, or from about 1.0 to about 1.6.

In some embodiments, the percent by weight of the second polymer within the particle is up to about 50% by weight (e.g., from about 4 to any of about 50%, about 5%, about 8%, about 10%, about 15%, about 20%, about 23%, about 25%, about 30%, about 35%, about 40%, about 45% or about 50% by weight). For example, the percent by weight of the second polymer within the particle is from about 3% to 30%, from about 5% to 25% or from about 8% to 23%. In some embodiments, the second polymer has a hydrophilic portion and a hydrophobic portion. In some embodiments, the second polymer is a block copolymer. In some embodiments, the second polymer comprises two regions, the two regions together being at least about 70% by weight of the polymer (e.g., at least about 80%, at least about 90%, at least about 95%). In some embodiments, the second polymer is a block copolymer comprising a hydrophobic polymer and a hydrophilic polymer. In some embodiments, the second polymer, e.g., a diblock copolymer, comprises a hydrophobic polymer and a hydrophilic polymer. In some embodiments, the second polymer, e.g., a triblock copolymer, comprises a hydrophobic polymer, a hydrophilic polymer and a hydrophobic polymer, e.g., PLA-PEG-PLA, PGA-PEG-PGA, PLGA-PEG-PLGA, PCL-PEG-PCL, PDO-PEG-PDO, PEG-PLGA-PEG, PLA-PEG-PGA, PGA-PEG-PLA, PLGA-PEG-PLA or PGA-PEG-PLGA.

In some embodiments, the hydrophobic portion of the second polymer is a biodegradable polymer (e.g., PLA, PGA, PLGA, PCL, PDO, polyanhydrides, polyorthoesters, or chitosan). In some embodiments, the hydrophobic portion of the second polymer is PLA. In some embodiments, the hydrophobic portion of the second polymer is PGA. In some embodiments, the hydrophobic portion of the second polymer is a copolymer of lactic and glycolic acid (e.g., PLGA). In some embodiments, the hydrophobic portion of the second polymer has a weight average molecular weight of from about 1 kDa to about 20 kDa (e.g., from about 1 kDa to about 18 kDa, 17 kDa, 16 kDa, 15 kDa, 14 kDa or 13 kDa, from about 2 kDa to about 12 kDa, from about 6 kDa to about 20 kDa, from about 5 kDa to about 18 kDa, from about 7 kDa to about 17 kDa, from about 8 kDa to about 13 kDa, from about 9 kDa to about 11 kDa, from about 10 kDa to about 14 kDa, from about 6 kDa to about 8 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa or about 17 kDa).

In some embodiments, the hydrophilic polymer portion of the second polymer is PEG. In some embodiments, the hydrophilic portion of the second polymer has a weight average molecular weight of from about 1 kDa to about 21 kDa (e.g., from about 1 kDa to about 3 kDa, e.g., about 2 kDa, or from about 2 kDa to about 5 kDa, e.g., about 3.5 kDa, or from about 4 kDa to about 6 kDa, e.g., about 5 kDa). In some embodiments, the ratio of weight average molecular weight of the hydrophilic to hydrophobic polymer portions of the second polymer is from about 1:1 to about 1:20 (e.g., about 1:4 to about 1:10, about 1:4 to about 1:7, about 1:3 to about 1:7, about 1:3 to about 1:6, about 1:4 to about 1:6.5 (e.g., 1:4, 1:4.5, 1:5, 1:5.5, 1:6, 1:6.5) or about 1:1 to about 1:4 (e.g., about 1:1.4, 1:1.8, 1:2, 1:2.4, 1:2.8, 1:3, 1:3.2, 1:3.5 or 1:4). In one embodiment, the hydrophilic portion of the second polymer has a weight average molecular weight of from about 2 kDa to 3.5 kDa and the ratio of the weight average molecular weight of the hydrophilic to hydrophobic portions of the second polymer is from about 1:4 to about 1:6.5 (e.g., 1:4, 1:4.5, 1:5, 1:5.5, 1:6, 1:6.5). In one embodiment, the hydrophilic portion of the second polymer has a weight average molecular weight of from about 4 kDa to 6 kDa (e.g., 5 kDa) and the ratio of the weight average molecular weight of the hydrophilic to hydrophobic portions of the second polymer is from about 1:1 to about 1:3.5 (e.g., about 1:1.4, 1:1.8, 1:2, 1:2.4, 1:2.8, 1:3, 1:3.2, or 1:3.5).

In some embodiments, the hydrophilic polymer portion of the second polymer has a terminal hydroxyl moiety. In some embodiments, the hydrophilic polymer portion of the second polymer has a terminal alkoxy moiety. In some embodiments, the hydrophilic polymer portion of the second polymer is a methoxy PEG (e.g., a terminal methoxy PEG). In some embodiments, the hydrophilic polymer portion of the second polymer does have a terminal alkoxy moiety. In some embodiments, the terminus of the hydrophilic polymer portion of the second polymer is conjugated to a hydrophobic polymer, e.g., to make a triblock copolymer.

In some embodiments, the hydrophilic polymer portion of the second polymer comprises a terminal conjugate. In some embodiments, the terminal conjugate is a targeting agent or a dye. In some embodiments, the terminal conjugate is a folate or a rhodamine. In some embodiments, the terminal conjugate is a targeting peptide (e.g., an RGD peptide).

In some embodiments, the hydrophilic polymer portion of the second polymer is attached to the hydrophobic polymer portion through a covalent bond. In some embodiments, the hydrophilic polymer is attached to the hydrophobic polymer through an amide, ester, ether, amino, carbamate, or carbonate bond (e.g., an ester or an amide).

In some embodiments, the ratio by weight of the first to the second polymer is from about 1:1 to about 20:1, e.g., about 1:1 to about 10:1, e.g., about 1:1 to 9:1, or about 1.2: to 8:1. In some embodiments, the ratio of the first and second polymer is from about 85:15 to about 55:45 percent by weight or about 84:16 to about 60:40 percent by weight. In some embodiments, the ratio by weight of the first polymer to the compound comprising at least one acidic moiety is from about 1:3 to about 1000:1, e.g., about 1:1 to about 10:1, or about 1.5:1. In some embodiments, the ratio by weight of the second polymer to the compound comprising at least one acidic moiety is from about 1:10 to about 250:1, e.g., from about 1:5 to about 5:1, or from about 1:3.5 to about 1:1.

In some embodiments the particle is substantially free of a targeting agent (e.g., of a targeting agent covalently linked to a component of the particle, e.g., to the first or second polymer or agent), e.g., a targeting agent able to bind to or otherwise associate with a target biological entity, e.g., a membrane component, a cell surface receptor, prostate specific membrane antigen, or the like. In some embodiments the particle is substantially free of a targeting agent that causes the particle to become localized to a tumor, a disease site, a tissue, an organ, a type of cell, e.g., a cancer cell, within the body of a subject to whom a therapeutically effective amount of the particle is administered. In some embodiments, the particle is substantially free of a targeting agent selected from nucleic acid aptamers, growth factors, hormones, cytokines, interleukins, antibodies, integrins, fibronectin receptors, p-glycoprotein receptors, peptides and cell binding sequences. In some embodiments, no polymer is conjugated to a targeting moiety. In an embodiment substantially free of a targeting agent means substantially free of any moiety other than the first polymer, the second polymer, a third polymer (if present), a surfactant (if present), and the agent, e.g., an epothilone or anti-cancer agent, that targets the particle. Thus, in such embodiments, any contribution to localization by the first polymer, the second polymer, a third polymer (if present), a surfactant (if present), and the agent is not considered to be “targeting.” In an embodiment the particle is free of moieties added for the purpose of selectively targeting the particle to a site in a subject, e.g., by the use of a moiety on the particle having a high and specific affinity for a target in the subject.

In some embodiments the second polymer is other than a lipid, e.g., other than a phospholipid. In some embodiments the particle is substantially free of an amphiphilic layer that reduces water penetration into the nanoparticle. In some embodiment the particle comprises less than 5 or 10% (e.g., as determined as w/w, v/v) of a lipid, e.g., a phospholipid. In some embodiments the particle is substantially free of a lipid layer, e.g., a phospholipid layer, e.g., that reduces water penetration into the nanoparticle. In some embodiments the particle is substantially free of lipid, e.g., is substantially free of phospholipid.

In some embodiments the agent is covalently bound to a PLGA polymer.

In some embodiments the particle is substantially free of a radiopharmaceutical agent, e.g., a radiotherapeutic agent, radiodiagnostic agent, prophylactic agent, or other radioisotope. In some embodiments the particle is substantially free of an immunomodulatory agent, e.g., an immunostimulatory agent or immunosuppressive agent. In some embodiments the particle is substantially free of a vaccine or immunogen, e.g., a peptide, sugar, lipid-based immunogen, B cell antigen or T cell antigen. In some embodiments, the particle is substantially free of water soluble PLGA (e.g., PLGA having a weight average molecular weight of less than about 1 kDa).

In some embodiments, the ratio of the first polymer to the second polymer is such that the particle comprises at least 5%, 8%, 10%, 12%, 15%, 18%, 20%, 23%, 25%, or 30% by weight of a polymer having a hydrophobic portion and a hydrophilic portion.

In some embodiments, the zeta potential of the particle surface, when measured in water, is from about −80 mV to about 50 mV, e.g., about −50 mV to about 30 mV, about −20 mV to about 20 mV, or about −10 mV to about 10 mV. In some embodiments, the zeta potential of the particle surface, when measured in water, is neutral or slightly negative. In some embodiments, the zeta potential of the particle surface, when measured in water, is less than 0, e.g., about 0 mV to about −20 mV.

In some embodiments, the particle comprises less than 5000 ppm of a solvent (e.g., acetone, tert-butylmethyl ether, heptane, dichloromethane, dimethylformamide, ethyl acetate, acetonitrile, tetrahydrofuran, ethanol, methanol, isopropyl alcohol, methyl ethyl ketone, butyl acetate, or propyl acetate), (e.g., less than 4500 ppm, less than 4000 ppm, less than 3500 ppm, less than 3000 ppm, less than 2500 ppm, less than 2000 ppm, less than 1500 ppm, less than 1000 ppm, less than 500 ppm, less than 250 ppm, less than 100 ppm, less than 50 ppm, less than 25 ppm, less than 10 ppm, less than 5 ppm, less than 2 ppm, or less than 1 ppm). In some embodiments, the particle is substantially free of a solvent (e.g., acetone, tert-butylmethyl ether, heptane, dichloromethane, dimethylformamide, ethyl acetate, acetonitrile, tetrahydrofuran, ethanol, methanol, isopropyl alcohol, methyl ethyl ketone, butyl acetate, or propyl acetate).

In some embodiments, the particle is substantially free of a class II or class III solvent as defined by the United States Department of Health and Human Services Food and Drug Administration “Q3c-Tables and List.” In some embodiments, the particle comprises less than 5000 ppm of acetone. In some embodiments, the particle comprises less than 5000 ppm of tert-butylmethyl ether. In some embodiments, the particle comprises less than 5000 ppm of heptane. In some embodiments, the particle comprises less than 600 ppm of dichloromethane. In some embodiments, the particle comprises less than 880 ppm of dimethylformamide. In some embodiments, the particle comprises less than 5000 ppm of ethyl acetate. In some embodiments, the particle comprises less than 410 ppm of acetonitrile. In some embodiments, the particle comprises less than 720 ppm of tetrahydrofuran. In some embodiments, the particle comprises less than 5000 ppm of ethanol. In some embodiments, the particle comprises less than 3000 ppm of methanol. In some embodiments, the particle comprises less than 5000 ppm of isopropyl alcohol. In some embodiments, the particle comprises less than 5000 ppm of methyl ethyl ketone. In some embodiments, the particle comprises less than 5000 ppm of butyl acetate. In some embodiments, the particle comprises less than 5000 ppm of propyl acetate.

In some embodiments, a composition comprising a plurality of particles is substantially free of solvent.

In some embodiments, in a composition of a plurality of particles, the particles have an average diameter of from about 50 nm to about 500 nm (e.g., from about 50 to about 200 nm). In some embodiments, in a composition of a plurality of particles, the particles have a Dv50 (median particle size) from about 50 nm to about 220 nm (e.g., from about 75 nm to about 200 nm). In some embodiments, in a composition of a plurality of particles, the particles have a Dv90 (particle size below which 90% of the volume of particles exists) of about 50 nm to about 500 nm (e.g., about 75 nm to about 220 nm).

In some embodiments, a single agent is attached to a single first polymer, e.g., to a terminal end of the polymer. In some embodiments, a plurality of agents are attached to a single first polymer (e.g., 2, 3, 4, 5, 6, or more). In some embodiments, the agents are the same agent. In some embodiments, the agents are different agents.

In some embodiments, the agent is an epothilone selected from ixabepilone, epothilone B, epothilone D, BMS310705, dehydelone and ZK-EPO. In some embodiments, the agent is an epothilone described herein.

In some embodiments, the agent is an epothilone attached to the polymer via the hydroxyl group at the 3 position. In some embodiments, the agent is an epothilone attached to the polymer via the hydroxyl group at the 7 position.

In some embodiments, the agent is attached directly to the polymer, e.g., through a covalent bond. In some embodiments, the agent is attached to a terminal end of the polymer via an amide, ester, ether, amino, carbamate or carbonate bond. In some embodiments, the agent is attached to a terminal end of the polymer. In some embodiments, the polymer comprises one or more side chains and the agent is directly attached to the polymer through one or more of the side chains.

In some embodiments, the polymer-agent conjugate in the particle, e.g., the nanoparticle, is:

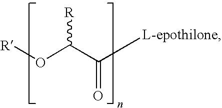

wherein L is a bond or linker, e.g., a linker described herein; and

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the epothilone is selected from ixabepilone, epothilone B, epothilone D, BMS310705, dehydelone and ZK-EPO. In some embodiments, the agent is an epothilone described herein.

In some embodiments, L is a bond.

In some embodiments, L is a linker, e.g., a linker described herein.

In some embodiments, the linker is an alkanoate linker. In some embodiments, the linker is a PEG-based linker. In some embodiments, the linker comprises a disulfide bond. In some embodiments, the linker is a self-immolative linker. In some embodiments, the linker is an amino acid or a peptide (e.g., glutamic acid such as L-glutamic acid, D-glutamic acid, DL-glutamic acid or β-glutamic acid, branched glutamic acid or polyglutamic acid). In some embodiments, the linker is β-alanine glycolate.

In some embodiments, the polymer-agent conjugate in the particle, e.g., the nanoparticle, is:

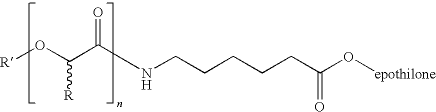

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the epothilone is selected from ixabepilone, epothilone B, epothilone D, BMS310705, dehydelone and ZK-EPO. In some embodiments, the agent is an epothilone described herein.

In some embodiments, the polymer-agent conjugate in the particle, e.g., the nanoparticle, is:

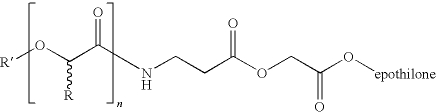

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the epothilone is selected from ixabepilone, epothilone B, epothilone D, BMS310705, dehydelone and ZK-EPO. In some embodiments, the agent is an epothilone described herein.

In some embodiments the linker is a multifunctional linker. In some embodiments, the multifunctional linker has 2, 3, 4, 5, 6 or more reactive moieties that may be functionalized with an agent. In some embodiments, all reactive moieties are functionalized with an agent. In some embodiments, not all of the reactive moieties are functionalized with an agent (e.g., the multifunctional linker has two reactive moieties, and only one reacts with an agent; or the multifunctional linker has four reactive moieties, and only one, two or three react with an agent.)

In some embodiments, two agents are attached to a polymer via a multifunctional linker. In some embodiments, the two agents are the same agent. In some embodiments, the two agents are different agents. In some embodiments, the agent is covalently attached to the polymer via a glutamate linker.

In some embodiments, the polymer-agent conjugate in the particle, e.g., the nanoparticle, is:

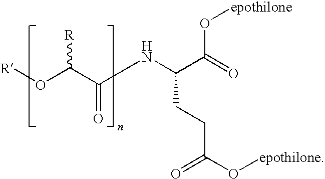

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, each epothilone is independently selected from ixabepilone, epothilone B, epothilone D, BMS310705, dehydelone and ZK-EPO. In some embodiments, each epothilone is independently selected from the epothilones described herein.

In some embodiments, at least one epothilone is attached to the polymer via the hydroxyl group at the 3 position. In some embodiments, at least one epothilone is attached to the polymer via the hydroxyl group at the 7 position. In some embodiments, each epothilone is attached via the same hydroxyl group, e.g., the hydroxyl group at the 3 position or the hydroxyl group at the 7 position. In some embodiments, each epothilone is attached via the hydroxyl group at the 3 position. In some embodiments, each epothilone is attached via the hydroxyl group at the 7 position. In some embodiments, the epothilone molecules may be attached via different hydroxyl groups, e.g., one epothilone is attached via the hydroxyl group at the 3 position and the other epothilone is attached via the hydroxyl group at the 7 position.

In some embodiments, four agents are attached to a polymer via a multifunctional linker. In some embodiments, the four agents are the same agent. In some embodiments, the four agents are different agents. In some embodiments, the agent is covalently attached to the polymer via a tri(glutamate) linker.

In some embodiments, the polymer-agent conjugate in the particle, e.g., the nanoparticle, is:

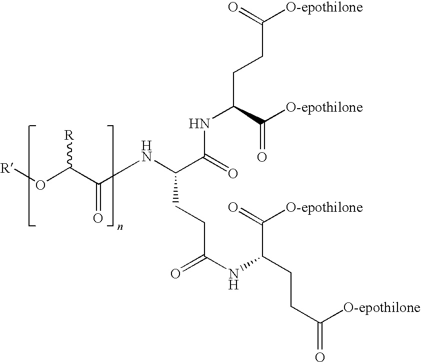

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, each epothilone is independently selected from ixabepilone, epothilone B, epothilone D, BMS310705, dehydelone and ZK-EPO. In some embodiments, each epothilone is independently selected from the epothilones described herein.

In some embodiments, at least one epothilone is attached to the polymer via the hydroxyl group at the 3 position. In some embodiments, at least one epothilone is attached to the polymer via the hydroxyl group at the 7 position. In some embodiments, each epothilone is attached via the same hydroxyl group, e.g., the hydroxyl group at the 3 position or the hydroxyl group at the 7 position. In some embodiments, each epothilone is attached via the hydroxyl group at the 3 position. In some embodiments, each epothilone is attached via the hydroxyl group at the 7 position. In some embodiments, the epothilone molecules may be attached via different hydroxyl groups, e.g., three epothilones are attached via the hydroxyl group at the 3 position and the other epothilone is attached via the hydroxyl group at the 7 position.

In some embodiments, the particle comprises a plurality of polymer-agent conjugates. In some embodiments, the plurality of polymer-agent conjugates have the same polymer and the same agent, and differ in the nature of the linkage between the agent and the polymer. For example, in some embodiments, the polymer is PLGA, and the plurality of polymer-agent conjugates includes PLGA polymers attached to an epothilone via the hydroxyl group at the 3 position, and PLGA polymers attached to an epothilone via the hydroxyl group at the 7 position. In some embodiments, the polymer is PLGA, and the plurality of polymer-agent conjugates includes epothilone molecules attached to more than one polymer chain, e.g., epothilone molecules with PLGA polymers attached to the hydroxyl group at the 3 position and the hydroxyl group at the 7 position.

In some embodiments, the plurality of polymer-agent conjugates have the same polymer and the same agent, but the agent may be attached to the polymer via different linkers. In some embodiments, the plurality of polymer-agent conjugates includes a polymer directly attached to an agent and a polymer attached to an agent via a linker. In an embodiment, one agent is released from one polymer-agent conjugate in the plurality with a first release profile and a second agent is released from a second polymer-agent conjugate in the plurality with a second release profile. E.g., a bond between the first agent and the first polymer is more rapidly broken than a bond between the second agent and the second polymer. E.g., the first polymer-agent conjugate can comprise a first linker linking the first agent to the first polymer and the second polymer-agent conjugate can comprise a second linker linking the second agent to the second polymer, wherein the linkers provide for different profiles for release of the first and second agents from their respective agent-polymer conjugates.

In some embodiments, the plurality of polymer-agent conjugates includes different polymers. In some embodiments, the plurality of polymer-agent conjugates includes different agents.

In some embodiments, the agent is present in the particle in an amount of from about 1 to about 30% by weight (e.g., from about 3 to about 30% by weight, from about 4 to about 25% by weight, or from about 5 to about 13%, 14%, 15%, 16%, 17%, 18%, 19% or 20% by weight).

In an embodiment the particle comprises the enumerated elements.

In an embodiment the particle consists of the enumerated elements.

In an embodiment the particle consists essentially of the enumerated elements.

In one yet another aspect, the invention features a particle. The particle comprises:

a first polymer,

a second polymer having a hydrophilic portion and a hydrophobic portion,

a first agent attached to the first polymer or second polymer to form a polymer-agent conjugate, and

a second agent embedded in the particle,

wherein at least one of the first or second agent is an epothilone.

In some embodiments, the epothilone is selected from ixabepilone, epothilone B, epothilone D, BMS310705, dehydelone and ZK-EPO. In some embodiments, the epothilone is an epothilone described herein. In some embodiments, at least one of the first or second agent is an epothilone, and the other of the first or second agent is an anti-cancer agent, e.g., an anti-cancer agent described herein. In some embodiments, the anti-cancer agent is an agent other than an epothilone.

In some embodiments, the second agent embedded in the particle makes up from about 0.1 to about 10% by weight of the particle (e.g., about 0.5% wt., about 1% wt., about 2% wt., about 3% wt., about 4% wt., about 5% wt., about 6% wt., about 7% wt., about 8% wt., about 9% wt., about 10% wt.).

In some embodiments, the second agent embedded in the particle is substantially absent from the surface of the particle. In some embodiments, the second agent embedded in the particle is substantially uniformly distributed throughout the particle. In some embodiments, the second agent embedded in the particle is not uniformly distributed throughout the particle. In some embodiments, the particle includes hydrophobic pockets and the embedded second agent is concentrated in hydrophobic pockets of the particle.

In some embodiments, the second agent embedded in the particle forms one or more non-covalent interactions with a polymer in the particle. In some embodiments, the second agent forms one or more hydrophobic interactions with a hydrophobic polymer in the particle. In some embodiments, the second agent forms one or more hydrogen bonds with a polymer in the particle.

In some embodiments, the particle is a nanoparticle. In some embodiments, the nanoparticle has a diameter of less than or equal to about 220 nm (e.g., less than or equal to about 215 nm, 210 nm, 205 nm, 200 nm, 195 nm, 190 nm, 185 nm, 180 nm, 175 nm, 170 nm, 165 nm, 160 nm, 155 nm, 150 nm, 145 nm, 140 nm, 135 nm, 130 nm, 125 nm, 120 nm, 115 nm, 110 nm, 105 nm, 100 nm, 95 nm, 90 nm, 85 nm, 80 nm, 75 nm, 70 nm, 65 nm, 60 nm, 55 nm or 50 nm).

In some embodiments, the particle further comprises a compound comprising at least one acidic moiety, wherein the compound is a polymer or a small molecule.

In some embodiments, the compound comprising at least one acidic moiety is a polymer comprising an acidic group. In some embodiments, the compound comprising at least one acidic moiety is a hydrophobic polymer. In some embodiments, the first polymer and the compound comprising at least one acidic moiety are the same polymer. In some embodiments, the compound comprising at least one acidic moiety is PLGA. In some embodiments, the ratio of lactic acid monomers to glycolic acid monomers in PLGA is from about 0.1:99.9 to about 99.9:0.1. In some embodiments, the ratio of lactic acid monomers to glycolic acid monomers in PLGA is from about 75:25 to about 25:75, e.g., about 60:40 to about 40:60 (e.g., about 50:50), about 60:40, or about 75:25. In some embodiments, the PLGA comprises a terminal hydroxyl group. In some embodiments, the PLGA comprises a terminal acyl group (e.g., an acetyl group).

In some embodiments, the weight average molecular weight of the compound comprising at least one acidic moiety is from about 1 kDa to about 20 kDa (e.g., from about 1 kDa to about 15 kDa, from about 2 kDa to about 12 kDa, from about 6 kDa to about 20 kDa, from about 5 kDa to about 15 kDa, from about 7 kDa to about 11 kDa, from about 5 kDa to about 10 kDa, from about 7 kDa to about 10 kDa, from about 5 kDa to about 7 kDa, from about 6 kDa to about 8 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa or about 17 kDa). In some embodiments, the compound comprising at least one acidic moiety has a glass transition temperature of from about 20° C. to about 60° C.

In some embodiments, the compound comprising at least one acidic moiety has a polymer polydispersity index of less than or equal to about 2.5 (e.g., less than or equal to about 2.2, or less than or equal to about 2.0). In some embodiments, the compound comprising at least one acidic moiety has a polymer polydispersity index of about 1.0 to about 2.5, e.g., from about 1.0 to about 2.0, from about 1.0 to about 1.8, from about 1.0 to about 1.7, or from about 1.0 to about 1.6.

In some embodiments, the particle comprises a plurality of compounds comprising at least one acidic moiety. For example, in some embodiments, one compound of the plurality of compounds comprising at least one acidic moiety is a PLGA polymer wherein the hydroxy terminus is functionalized with an acetyl group, and another compound in the plurality is a PLGA polymer wherein the hydroxy terminus is unfunctionalized.

In some embodiments, the percent by weight of the compound comprising at least one acidic moiety within the particle is up to about 50% (e.g., up to about 45% by weight, up to about 40% by weight, up to about 35% by weight, up to about 30% by weight, from about 0 to about 30% by weight, e.g., about 4.5%, about 9%, about 12%, about 15%, about 18%, about 20%, about 22%, about 24%, about 26%, about 28% or about 30%).

In some embodiments, the compound comprising at least one acidic moiety is a small molecule comprising an acidic group.

In some embodiments, the particle further comprises a surfactant. In some embodiments, the surfactant is PEG, PVA, PVP, poloxamer, a polysorbate, a polyoxyethylene ester, a PEG-lipid (e.g., PEG-ceramide, d-alpha-tocopheryl polyethylene glycol 1000 succinate), 1,2-Distearoyl-sn-Glycero-3-[Phospho-rac-(1-glycerol)] or lecithin. In some embodiments, the surfactant is PVA and the PVA is from about 3 kDa to about 50 kDa (e.g., from about 5 kDa to about 45 kDa, about 7 kDa to about 42 kDa, from about 9 kDa to about 30 kDa, or from about 11 to about 28 kDa) and up to about 98% hydrolyzed (e.g., about 75-95%, about 80-90% hydrolyzed, or about 85% hydrolyzed). In some embodiments, the surfactant is polysorbate 80. In some embodiments, the surfactant is Solutol® HS 15. In some embodiments, the surfactant is present in an amount of up to about 35% by weight of the particle (e.g., up to about 20% by weight or up to about 25% by weight, from about 15% to about 35% by weight, from about 20% to about 30% by weight, or from about 23% to about 26% by weight).

In some embodiments, the particle further comprises a stabilizer or lyoprotectant, e.g., a stabilizer or lyoprotectant described herein. In some embodiments, the stabilizer or lyoprotectant is a carbohydrate (e.g., a carbohydrate described herein, such as, e.g., sucrose, cyclodextrin or a derivative of cyclodextrin (e.g. 2-hydroxypropyl-β-cyclodextrin)), salt, PEG, PVP or crown ether.

In some embodiments, the first agent and the second agent are the same agent (e.g., both the first and second agents are ixabepilone). In some embodiments, the first agent and the second agent are different agents (e.g., one agent is ixabepilone and the other is epothilone B; or one agent is ixabepilone and the other is docetaxel).

In some embodiments, the first agent is attached to the first polymer to form a polymer-agent conjugate. In some embodiments, first agent is attached to the second polymer to form a polymer-agent conjugate.

In some embodiments, the second agent is not covalently bound to the first or second polymer.

In an embodiment the amount of the first agent in the particle that is not attached to the first polymer is less than about 5% (e.g., less than about 2% or less than about 1%, e.g., in terms of w/w or number/number) of the amount of the first agent attached to the first polymer.

In some embodiments, the first polymer is a biodegradable polymer (e.g., PLA, PGA, PLGA, PCL, PDO, polyanhydrides, polyorthoesters or chitosan). In some embodiments, the first polymer is a hydrophobic polymer. In some embodiments, the percent by weight of the first polymer within the particle is from about 40% to about 90%, e.g., about 30% to about 70%. In some embodiments, the first polymer is PLA. In some embodiments, the first polymer is PGA.

In some embodiments, the first polymer is a copolymer of lactic and glycolic acid (e.g., PLGA). In some embodiments, the first polymer is a PLGA-ester. In some embodiments, the first polymer is a PLGA-lauryl ester. In some embodiments, the first polymer comprises a terminal free acid. In some embodiments, the first polymer comprises a terminal acyl group (e.g., an acetyl group). In some embodiments, the polymer comprises a terminal hydroxyl group. In some embodiments, the ratio of lactic acid monomers to glycolic acid monomers in PLGA is from about 0.1:99.9 to about 99.9:0.1. In some embodiments, the ratio of lactic acid monomers to glycolic acid monomers in PLGA is from about 75:25 to about 25:75, e.g., about 60:40 to about 40:60 (e.g., about 50:50), about 60:40, or about 75:25.

In some embodiments, the weight average molecular weight of the first polymer is from about 1 kDa to about 20 kDa (e.g., from about 1 kDa to about 15 kDa, from about 2 kDa to about 12 kDa, from about 6 kDa to about 20 kDa, from about 5 kDa to about 15 kDa, from about 7 kDa to about 11 kDa, from about 5 kDa to about 10 kDa, from about 7 kDa to about 10 kDa, from about 5 kDa to about 7 kDa, from about 6 kDa to about 8 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa or about 17 kDa). In some embodiments, the first polymer has a glass transition temperature of from about 20° C. to about 60° C. In some embodiments, the first polymer has a polymer polydispersity index of less than or equal to about 2.5 (e.g., less than or equal to about 2.2, or less than or equal to about 2.0). In some embodiments, the first polymer has a polymer polydispersity index of about 1.0 to about 2.5, e.g., from about 1.0 to about 2.0, from about 1.0 to about 1.8, from about 1.0 to about 1.7, or from about 1.0 to about 1.6.

In some embodiments, the percent by weight of the second polymer within the particle is up to about 50% by weight (e.g., from about 4 to any of about 50%, about 5%, about 8%, about 10%, about 15%, about 20%, about 23%, about 25%, about 30%, about 35%, about 40%, about 45% or about 50% by weight). For example, the percent by weight of the second polymer within the particle is from about 3% to 30%, from about 5% to 25% or from about 8% to 23%. In some embodiments, the second polymer has a hydrophilic portion and a hydrophobic portion. In some embodiments, the second polymer is a block copolymer. In some embodiments, the second polymer comprises two regions, the two regions together being at least about 70% by weight of the polymer (e.g., at least about 80%, at least about 90%, at least about 95%). In some embodiments, the second polymer is a block copolymer comprising a hydrophobic polymer and a hydrophilic polymer. In some embodiments, the second polymer is diblock copolymer comprising a hydrophobic polymer and a hydrophilic polymer. In some embodiments, the second polymer, e.g., a diblock copolymer, comprises a hydrophobic polymer and a hydrophilic polymer. In some embodiments, the second polymer, e.g., a triblock copolymer, comprises a hydrophobic polymer, a hydrophilic polymer and a hydrophobic polymer, e.g., PLA-PEG-PLA, PGA-PEG-PGA, PLGA-PEG-PLGA, PCL-PEG-PCL, PDO-PEG-PDO, PEG-PLGA-PEG, PLA-PEG-PGA, PGA-PEG-PLA, PLGA-PEG-PLA or PGA-PEG-PLGA.

In some embodiments, the hydrophobic portion of the second polymer is a biodegradable polymer (e.g., PLA, PGA, PLGA, PCL, PDO, polyanhydrides, polyorthoesters or chitosan). In some embodiments, the hydrophobic portion of the second polymer is PLA. In some embodiments, the hydrophobic portion of the second polymer is PGA. In some embodiments, the hydrophobic portion of the second polymer is a copolymer of lactic and glycolic acid (e.g., PLGA). In some embodiments, the hydrophobic portion of the second polymer has a weight average molecular weight of from about 1 kDa to about 20 kDa (e.g., from about 1 kDa to about 18 kDa, 17 kDa, 16 kDa, 15 kDa, 14 kDa or 13 kDa, from about 2 kDa to about 12 kDa, from about 6 kDa to about 20 kDa, from about 5 kDa to about 18 kDa, from about 7 kDa to about 17 kDa, from about 8 kDa to about 13 kDa, from about 9 kDa to about 11 kDa, from about 10 kDa to about 14 kDa, from about 6 kDa to about 8 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa or about 17 kDa).

In some embodiments, the hydrophilic polymer portion of the second polymer is PEG. In some embodiments, the hydrophilic portion of the second polymer has a weight average molecular weight of from about 1 kDa to about 21 kDa (e.g., from about 1 kDa to about 3 kDa, e.g., about 2 kDa, or from about 2 kDa to about 5 kDa, e.g., about 3.5 kDa, or from about 4 kDa to about 6 kDa, e.g., about 5 kDa). In some embodiments, the ratio of weight average molecular weight of the hydrophilic to hydrophobic polymer portions of the second polymer is from about 1:1 to about 1:20 (e.g., about 1:4 to about 1:10, about 1:4 to about 1:7, about 1:3 to about 1:7, about 1:3 to about 1:6, about 1:4 to about 1:6.5 (e.g., 1:4, 1:4.5, 1:5, 1:5.5, 1:6, 1:6.5) or about 1:1 to about 1:4 (e.g., about 1:1.4, 1:1.8, 1:2, 1:2.4, 1:2.8, 1:3, 1:3.2, 1:3.5 or 1:4). In one embodiment, the hydrophilic portion of the second polymer has a weight average molecular weight of from about 2 kDa to 3.5 kDa and the ratio of the weight average molecular weight of the hydrophilic to hydrophobic portions of the second polymer is from about 1:4 to about 1:6.5 (e.g., 1:4, 1:4.5, 1:5, 1:5.5, 1:6, 1:6.5). In one embodiment, the hydrophilic portion of the second polymer has a weight average molecular weight of from about 4 kDa to 6 kDa (e.g., 5 kDa) and the ratio of the weight average molecular weight of the hydrophilic to hydrophobic portions of the second polymer is from about 1:1 to about 1:3.5 (e.g., about 1:1.4, 1:1.8, 1:2, 1:2.4, 1:2.8, 1:3, 1:3.2, or 1:3.5).

In some embodiments, the hydrophilic polymer portion of the second polymer has a terminal hydroxyl moiety. In some embodiments, the hydrophilic polymer portion of the second polymer has a terminal alkoxy moiety. In some embodiments, the hydrophilic polymer portion of the second polymer is a methoxy PEG (e.g., a terminal methoxy PEG). In some embodiments, the hydrophilic polymer portion of the second polymer does not have a terminal alkoxy moiety. In some embodiments, the terminus of the hydrophilic polymer portion of the second polymer is conjugated to a hydrophobic polymer, e.g., to make a triblock copolymer.

In some embodiments, the hydrophilic polymer portion of the second polymer comprises a terminal conjugate. In some embodiments, the terminal conjugate is a targeting agent or a dye. In some embodiments, the terminal conjugate is a folate or a rhodamine. In some embodiments, the terminal conjugate is a targeting peptide (e.g., an RGD peptide).

In some embodiments, the hydrophilic polymer portion of the second polymer is attached to the hydrophobic polymer portion through a covalent bond. In some embodiments, the hydrophilic polymer is attached to the hydrophobic polymer through an amide, ester, ether, amino, carbamate, or carbonate bond (e.g., an ester or an amide).

In some embodiments, the ratio by weight of the first to the second polymer is from about 1:1 to about 20:1, e.g., about 1:1 to about 10:1, e.g., about 1:1 to 9:1, or about 1.2: to 8:1. In some embodiments, the ratio of the first and second polymer is from about 85:15 to about 55:45 percent by weight or about 84:16 to about 60:40 percent by weight. In some embodiments, the ratio by weight of the first polymer to the compound comprising at least one acidic moiety is from about 1:3 to about 1000:1, e.g., about 1:1 to about 10:1, or about 1.5:1. In some embodiments, the ratio by weight of the second polymer to the compound comprising at least one acidic moiety is from about 1:10 to about 250:1, e.g., from about 1:5 to about 5:1, or from about 1:3.5 to about 1:1.

In some embodiments the particle is substantially free of a targeting agent (e.g., of a targeting agent covalently linked to a component of the particle, e.g., to the first or second polymer or agent), e.g., a targeting agent able to bind to or otherwise associate with a target biological entity, e.g., a membrane component, a cell surface receptor, prostate specific membrane antigen, or the like. In some embodiments the particle is substantially free of a targeting agent that causes the particle to become localized to a tumor, a disease site, a tissue, an organ, a type of cell, e.g., a cancer cell, within the body of a subject to whom a therapeutically effective amount of the particle is administered. In some embodiments, the particle is substantially free of a targeting agent selected from nucleic acid aptamers, growth factors, hormones, cytokines, interleukins, antibodies, integrins, fibronectin receptors, p-glycoprotein receptors, peptides and cell binding sequences. In some embodiments, no polymer is conjugated to a targeting moiety. In an embodiment substantially free of a targeting agent means substantially free of any moiety other than the first polymer, the second polymer, a third polymer (if present), a surfactant (if present), and the agent, e.g., an epothilone or anti-cancer agent, that targets the particle. Thus, in such embodiments, any contribution to localization by the first polymer, the second polymer, a third polymer (if present), a surfactant (if present), and the agent is not considered to be “targeting.” In an embodiment the particle is free of moieties added for the purpose of selectively targeting the particle to a site in a subject, e.g., by the use of a moiety on the particle having a high and specific affinity for a target in the subject.

In some embodiments the second polymer is other than a lipid, e.g., other than a phospholipid. In some embodiments the particle is substantially free of an amphiphilic layer that reduces water penetration into the nanoparticle. In some embodiment the particle comprises less than 5 or 10% (e.g., as determined as w/w, v/v) of a lipid, e.g., a phospholipid. In some embodiments the particle is substantially free of a lipid layer, e.g., a phospholipid layer, e.g., that reduces water penetration into the nanoparticle. In some embodiments the particle is substantially free of lipid, e.g., is substantially free of phospholipid.

In some embodiments the first agent is covalently bound to a PLGA polymer.

In some embodiments the particle is substantially free of a radiopharmaceutical agent, e.g., a radiotherapeutic agent, radiodiagnostic agent, prophylactic agent, or other radioisotope. In some embodiments the particle is substantially free of an immunomodulatory agent, e.g., an immunostimulatory agent or immunosuppressive agent. In some embodiments the particle is substantially free of a vaccine or immunogen, e.g., a peptide, sugar, lipid-based immunogen, B cell antigen or T cell antigen. In some embodiments, the particle is substantially free of water soluble PLGA (e.g., PLGA having a weight average molecular weight of less than about 1 kDa).

In some embodiments, the ratio of the first polymer to the second polymer is such that the particle comprises at least 5%, 8%, 10%, 12%, 15%, 18%, 20%, 23%, 25% or 30% by weight of a polymer having a hydrophobic portion and a hydrophilic portion.

In some embodiments, the zeta potential of the particle surface, when measured in water, is from about −80 mV to about 50 mV, e.g., about −50 mV to about 30 mV, about −20 mV to about 20 mV, or about −10 mV to about 10 mV. In some embodiments, the zeta potential of the particle surface, when measured in water, is neutral or slightly negative. In some embodiments, the zeta potential of the particle surface, when measured in water, is less than 0, e.g., about 0 mV to about −20 mV.

In some embodiments, the particle comprises less than 5000 ppm of a solvent (e.g., acetone, tert-butylmethyl ether, heptane, dichloromethane, dimethylformamide, ethyl acetate, acetonitrile, tetrahydrofuran, ethanol, methanol, isopropyl alcohol, methyl ethyl ketone, butyl acetate, or propyl acetate), e.g., less than 4500 ppm, less than 4000 ppm, less than 3500 ppm, less than 3000 ppm, less than 2500 ppm, less than 2000 ppm, less than 1500 ppm, less than 1000 ppm, less than 500 ppm, less than 250 ppm, less than 100 ppm, less than 50 ppm, less than 25 ppm, less than 10 ppm, less than 5 ppm, less than 2 ppm, or less than 1 ppm). In some embodiments, the particle is substantially free of a solvent (e.g., acetone, tert-butylmethyl ether, heptane, dichloromethane, dimethylformamide, ethyl acetate, acetonitrile, tetrahydrofuran, ethanol, methanol, isopropyl alcohol, methyl ethyl ketone, butyl acetate, or propyl acetate).

In some embodiments, the particle is substantially free of a class II or class III solvent as defined by the United States Department of Health and Human Services Food and Drug Administration “Q3c-Tables and List.” In some embodiments, the particle comprises less than 5000 ppm of acetone. In some embodiments, the particle comprises less than 5000 ppm of tert-butylmethyl ether. In some embodiments, the particle comprises less than 5000 ppm of heptane. In some embodiments, the particle comprises less than 600 ppm of dichloromethane. In some embodiments, the particle comprises less than 880 ppm of dimethylformamide. In some embodiments, the particle comprises less than 5000 ppm of ethyl acetate. In some embodiments, the particle comprises less than 410 ppm of acetonitrile. In some embodiments, the particle comprises less than 720 ppm of tetrahydrofuran. In some embodiments, the particle comprises less than 5000 ppm of ethanol. In some embodiments, the particle comprises less than 3000 ppm of methanol. In some embodiments, the particle comprises less than 5000 ppm of isopropyl alcohol. In some embodiments, the particle comprises less than 5000 ppm of methyl ethyl ketone. In some embodiments, the particle comprises less than 5000 ppm of butyl acetate. In some embodiments, the particle comprises less than 5000 ppm of propyl acetate.

In some embodiments, a composition comprising a plurality of particles is substantially free of solvent.

In some embodiments, in a composition of a plurality of particles, the particles have an average diameter of from about 50 to about 500 nm (e.g., from about 50 to about 200 nm). In some embodiments, in a composition of a plurality of particles, the particles have a Dv50 (median particle size) from about 50 nm to about 220 nm (e.g., from about 75 nm to about 200 nm). In some embodiments, in a composition of a plurality of particles, the particles have a Dv90 (particle size below which 90% of the volume of particles exists) of about 50 nm to about 500 nm (e.g., about 75 nm to about 220 nm).

In some embodiments, a single first agent is attached to a single first polymer, e.g., to a terminal end of the polymer. In some embodiments, a plurality of first agents are attached to a single first polymer (e.g., 2, 3, 4, 5, 6, or more).

In some embodiments, the first agent is an epothilone selected from ixabepilone, epothilone B, epothilone D, BMS310705, dehydelone and ZK-EPO. In some embodiments, the first agent is an epothilone described herein. In some embodiments, the first agent is an anti-cancer agent.

In some embodiments, the first agent is an epothilone attached to the first polymer via the hydroxyl group at the 3 position. In some embodiments, the first agent is an epothilone attached to the first polymer via the hydroxyl group at the 7 position.

In some embodiments, the first agent is attached directly to the first polymer, e.g., through a covalent bond. In some embodiments, the first agent is attached to a terminal end of the first polymer via an amide, ester, ether, amino, carbamate or carbonate bond. In some embodiments, the first agent is attached to a terminal end of the first polymer. In some embodiments, the first polymer comprises one or more side chains and the first agent is directly attached to the first polymer through one or more of the side chains.

In some embodiments, the first agent is attached to the first polymer to form a polymer-agent conjugate.

In some embodiments, the polymer-agent conjugate in the particle, e.g., the nanoparticle, is:

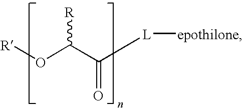

wherein L is a bond or linker, e.g., a linker described herein; and

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the epothilone is selected from ixabepilone, epothilone B, epothilone D, BMS310705, dehydelone and ZK-EPO. In some embodiments, the agent is an epothilone described herein.

In some embodiments, L is a bond.

In some embodiments, L is a linker, e.g., a linker described herein.

In some embodiments, the linker is an alkanoate linker. In some embodiments, the linker is a PEG-based linker. In some embodiments, the linker comprises a disulfide bond. In some embodiments, the linker is a self-immolative linker. In some embodiments, the linker is an amino acid or a peptide (e.g., glutamic acid such as L-glutamic acid, D-glutamic acid, DL-glutamic acid or β-glutamic acid, branched glutamic acid or polyglutamic acid). In some embodiments, the linker is β-alanine glycolate.

In some embodiments, the polymer-agent conjugate in the particle, e.g., the nanoparticle, is:

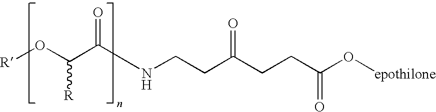

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the epothilone is selected from ixabepilone, epothilone B, epothilone D, BMS310705, dehydelone and ZK-EPO. In some embodiments, the agent is an epothilone described herein.

In some embodiments, the polymer-agent conjugate in the particle, e.g., the nanoparticle, is:

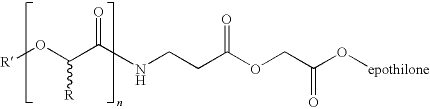

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the epothilone is selected from ixabepilone, epothilone B, epothilone D, BMS310705, dehydelone and ZK-EPO. In some embodiments, the agent is an epothilone described herein.

In some embodiments the linker is a multifunctional linker. In some embodiments, the multifunctional linker has 2, 3, 4, 5, 6 or more reactive moieties that may be functionalized with an agent. In some embodiments, all reactive moieties are functionalized with an agent. In some embodiments, not all of the reactive moieties are functionalized with an agent (e.g., the multifunctional linker has two reactive moieties, and only one reacts with an agent; or the multifunctional linker has four reactive moieties, and only one, two or three react with an agent.)

In some embodiments, two agents are attached to a polymer via a multifunctional linker. In some embodiments, the two agents are the same agent. In some embodiments, the two agents are different agents. In some embodiments, the agent is covalently attached to the polymer via a glutamate linker.

In some embodiments, the polymer-agent conjugate in the particle, e.g., the nanoparticle, is:

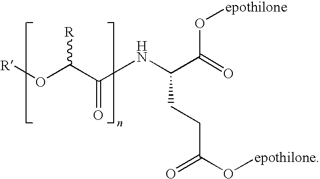

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, each epothilone is independently selected from ixabepilone, epothilone B, epothilone D, BMS310705, dehydelone and ZK-EPO. In some embodiments, each epothilone is independently selected from the epothilones described herein.

In some embodiments, at least one epothilone is attached to the polymer via the hydroxyl group at the 3 position. In some embodiments, at least one epothilone is attached to the polymer via the hydroxyl group at the 7 position. In some embodiments, each epothilone is attached via the same hydroxyl group, e.g., the hydroxyl group at the 3 position or the hydroxyl group at the 7 position. In some embodiments, each epothilone is attached via the hydroxyl group at the 3 position. In some embodiments, each epothilone is attached via the hydroxyl group at the 7 position. In some embodiments, the epothilone molecules may be attached via different hydroxyl groups, e.g., one epothilone is attached via the hydroxyl group at the 3 position and the other epothilone is attached via the hydroxyl group at the 7 position.

In some embodiments, four agents are attached to a polymer via a multifunctional linker. In some embodiments, the four agents are the same agent. In some embodiments, the four agents are different agents. In some embodiments, the agent is covalently attached to the polymer via a tri(glutamate) linker.

In some embodiments, the polymer-agent conjugate in the particle, e.g., the nanoparticle, is:

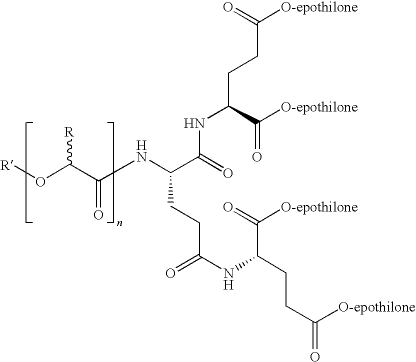

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, each epothilone is independently selected from ixabepilone, epothilone B, epothilone D, BMS310705, dehydelone and ZK-EPO. In some embodiments, each epothilone is independently selected from the epothilones described herein.

In some embodiments, at least one epothilone is attached to the polymer via the hydroxyl group at the 3 position. In some embodiments, at least one epothilone is attached to the polymer via the hydroxyl group at the 7 position. In some embodiments, each epothilone is attached via the same hydroxyl group, e.g., the hydroxyl group at the 3 position or the hydroxyl group at the 7 position. In some embodiments, each epothilone is attached via the hydroxyl group at the 3 position. In some embodiments, each epothilone is attached via the hydroxyl group at the 7 position. In some embodiments, the epothilone molecules may be attached via different hydroxyl groups, e.g., three epothilones are attached via the hydroxyl group at the 3 position and the other epothilone is attached via the hydroxyl group at the 7 position.

In some embodiments, the particle comprises a plurality of polymer-agent conjugates. In some embodiments, the plurality of polymer-agent conjugates have the same polymer and the same agent, and differ in the nature of the linkage between the agent and the polymer. For example, in some embodiments, the polymer is PLGA, and the plurality of polymer-agent conjugates includes PLGA polymers attached to an epothilone via the hydroxyl group at the 3 position, and PLGA polymers attached to an epothilone via the hydroxyl group at the 7 position. In some embodiments, the polymer is PLGA, and the plurality of polymer-agent conjugates includes epothilone molecules attached to more than one polymer chain, e.g., epothilone molecules with PLGA polymers attached to the hydroxyl group at the 3 position and the hydroxyl group at the 7 position.

In some embodiments, the plurality of polymer-agent conjugates have the same polymer and the same agent, but the agent may be attached to the polymer via different linkers. In some embodiments, the plurality of polymer-agent conjugates includes a polymer directly attached to an agent and a polymer attached to an agent via a linker. In an embodiment, one agent is released from one polymer-agent conjugate in the plurality with a first release profile and a second agent is released from a second polymer-agent conjugate in the plurality with a second release profile. E.g., a bond between the first agent and the first polymer is more rapidly broken than a bond between the second agent and the second polymer. E.g., the first polymer-agent conjugate can comprise a first linker linking the first agent to the first polymer and the second polymer-agent conjugate can comprise a second linker linking the second agent to the second polymer, wherein the linkers provide for different profiles for release of the first and second agents from their respective agent-polymer conjugates.

In some embodiments, the plurality of polymer-agent conjugates includes different polymers. In some embodiments, the plurality of polymer-agent conjugates includes different agents.

In some embodiments, the first agent is present in the particle in an amount of from about 1 to about 30% by weight (e.g., from about 3 to about 30% by weight, from about 4 to about 25% by weight, or from about 5 to about 13%, 14%, 15%, 16%, 17%, 18%, 19% or 20% by weight).

In some embodiments, the second agent is an epothilone selected from ixabepilone, epothilone B, epothilone D, BMS310705, dehydelone and ZK-EPO. In some embodiments, the second agent is an epothilone described herein. In some embodiments, the second agent is an anti-cancer agent.

In some embodiments, the second agent is in the form of a salt (e.g., an insoluble salt). In some embodiments, the second agent is in the form of a prodrug (i.e., the prodrug releases the agent in vivo). In some embodiments, the prodrug of the agent is conjugated to a hydrophobic moiety that is cleaved in vivo (e.g., a polymer or oligomer).

In some embodiments, at least about 50% of the second agent is embedded in the particle (e.g., embedded in the first polymer, second polymer, and/or compound comprising at least one acidic moiety). In some embodiments, substantially all of the second agent is embedded in the particle (e.g., embedded in the first polymer, second polymer, and/or compound comprising at least one acidic moiety).

In an embodiment the particle comprises the enumerated elements.

In an embodiment the particle consists of the enumerated elements.

In an embodiment the particle consists essentially of the enumerated elements.

In another aspect, the invention features a particle. The particle comprises:

a first polymer,

a second polymer having a hydrophilic portion and a hydrophobic portion, and

an agent embedded in the particle, wherein the agent is an epothilone.

In some embodiments, the agent embedded in the particle makes up from about 0.1 to about 10% by weight of the particle (e.g., about 0.5% wt., about 1% wt., about 2% wt., about 3% wt., about 4% wt., about 5% wt., about 6% wt., about 7% wt., about 8% wt., about 9% wt., about 10% wt.).

In some embodiments, the agent is substantially absent from the surface of the particle. In some embodiments, the agent is substantially uniformly distributed throughout the particle. In some embodiments, the agent is not uniformly distributed throughout the particle. In some embodiments, the particle includes hydrophobic pockets and the agent is concentrated in hydrophobic pockets of the particle.

In some embodiments, the agent forms one or more non-covalent interactions with a polymer in the particle. In some embodiments, the agent forms one or more hydrophobic interactions with a hydrophobic polymer in the particle. In some embodiments, the agent forms one or more hydrogen bonds with a polymer in the particle.

In some embodiments, the agent is not covalently bound to the first or second polymer.

In some embodiments, the particle is a nanoparticle. In some embodiments, the nanoparticle has a diameter of less than or equal to about 220 nm (e.g., less than or equal to about 215 nm, 210 nm, 205 nm, 200 nm, 195 nm, 190 nm, 185 nm, 180 nm, 175 nm, 170 nm, 165 nm, 160 nm, 155 nm, 150 nm, 145 nm, 140 nm, 135 nm, 130 nm, 125 nm, 120 nm, 115 nm, 110 nm, 105 nm, 100 nm, 95 nm, 90 nm, 85 nm, 80 nm, 75 nm, 70 nm, 65 nm, 60 nm, 55 nm or 50 nm).

In some embodiments, the particle further comprises a surfactant. In some embodiments, the surfactant is PEG, PVA, PVP, poloxamer, a polysorbate, a polyoxyethylene ester, a PEG-lipid (e.g., PEG-ceramide, d-alpha-tocopheryl polyethylene glycol 1000 succinate), 1,2-Distearoyl-sn-Glycero-3-[Phospho-rac-(1-glycerol)] or lecithin. In some embodiments, the surfactant is PVA and the PVA is from about 3 kDa to about 50 kDa (e.g., from about 5 kDa to about 45 kDa, about 7 kDa to about 42 kDa, from about 9 kDa to about 30 kDa, or from about 11 to about 28 kDa) and up to about 98% hydrolyzed (e.g., about 75-95%, about 80-90% hydrolyzed, or about 85% hydrolyzed). In some embodiments, the surfactant is polysorbate 80. In some embodiments, the surfactant is Solutol® HS 15. In some embodiments, the surfactant is present in an amount of up to about 35% by weight of the particle (e.g., up to about 20% by weight or up to about 25% by weight, from about 15% to about 35% by weight, from about 20% to about 30% by weight, or from about 23% to about 26% by weight).

In some embodiments, the particle further comprises a stabilizer or lyoprotectant, e.g., a stabilizer or lyoprotectant described herein. In some embodiments, the stabilizer or lyoprotectant is a carbohydrate (e.g., a carbohydrate described herein, such as, e.g., sucrose, cyclodextrin or a derivative of cyclodextrin (e.g. 2-hydroxypropyl-β-cyclodextrin)), salt, PEG, PVP or crown ether.

In some embodiments, the first polymer is a biodegradable polymer (e.g., PLA, PGA, PLGA, PCL, PDO, polyanhydrides, polyorthoesters or chitosan). In some embodiments, the first polymer is a hydrophobic polymer. In some embodiments, the percent by weight of the first polymer within the particle is from about 40% to about 90%. In some embodiments, the first polymer is PLA. In some embodiments, the first polymer is PGA.

In some embodiments, the first polymer is a copolymer of lactic and glycolic acid (e.g., PLGA). In some embodiments, the first polymer is a PLGA-ester. In some embodiments, the first polymer is a PLGA-lauryl ester. In some embodiments, the first polymer comprises a terminal free acid. In some embodiments, the first polymer comprises a terminal acyl group (e.g., an acetyl group). In some embodiments, the polymer comprises a terminal hydroxyl group. In some embodiments, the ratio of lactic acid monomers to glycolic acid monomers in PLGA is from about 0.1:99.9 to about 99.9:0.1. In some embodiments, the ratio of lactic acid monomers to glycolic acid monomers in PLGA is from about 75:25 to about 25:75, e.g., about 60:40 to about 40:60 (e.g., about 50:50), about 60:40, or about 75:25.

In some embodiments, the weight average molecular weight of the first polymer is from about 1 kDa to about 20 kDa (e.g., from about 1 kDa to about 15 kDa, from about 2 kDa to about 12 kDa, from about 6 kDa to about 20 kDa, from about 5 kDa to about 15 kDa, from about 7 kDa to about 11 kDa, from about 5 kDa to about 10 kDa, from about 7 kDa to about 10 kDa, from about 5 kDa to about 7 kDa, from about 6 kDa to about 8 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa or about 17 kDa). In some embodiments, the first polymer has a glass transition temperature of from about 20° C. to about 60° C. In some embodiments, the first polymer has a polymer polydispersity index of less than or equal to about 2.5 (e.g., less than or equal to about 2.2, or less than or equal to about 2.0). In some embodiments, the first polymer has a polymer polydispersity index of about 1.0 to about 2.5, e.g., from about 1.0 to about 2.0, from about 1.0 to about 1.8, from about 1.0 to about 1.7, or from about 1.0 to about 1.6.

In some embodiments, the percent by weight of the second polymer within the particle is up to about 50% by weight (e.g., from about 4 to any of about 50%, about 5%, about 8%, about 10%, about 15%, about 20%, about 23%, about 25%, about 30%, about 35%, about 40%, about 45% or about 50% by weight). For example, the percent by weight of the second polymer within the particle is from about 3% to 30%, from about 5% to 25% or from about 8% to 23%. In some embodiments, the second polymer has a hydrophilic portion and a hydrophobic portion. In some embodiments, the second polymer is a block copolymer. In some embodiments, the second polymer comprises two regions, the two regions together being at least about 70% by weight of the polymer (e.g., at least about 80%, at least about 90%, at least about 95%). In some embodiments, the second polymer is a block copolymer comprising a hydrophobic polymer and a hydrophilic polymer. In some embodiments, the second polymer is diblock copolymer comprising a hydrophobic polymer and a hydrophilic polymer. In some embodiments, the second polymer, e.g., a diblock copolymer, comprises a hydrophobic polymer and a hydrophilic polymer. In some embodiments, the second polymer, e.g., a triblock copolymer, comprises a hydrophobic polymer, a hydrophilic polymer and a hydrophobic polymer, e.g., PLA-PEG-PLA, PGA-PEG-PGA, PLGA-PEG-PLGA, PCL-PEG-PCL, PDO-PEG-PDO, PEG-PLGA-PEG, PLA-PEG-PGA, PGA-PEG-PLA, PLGA-PEG-PLA or PGA-PEG-PLGA.

In some embodiments, the hydrophobic portion of the second polymer is a biodegradable polymer (e.g., PLA, PGA, PLGA, PCL, PDO, polyanhydrides, polyorthoesters or chitosan). In some embodiments, the hydrophobic portion of the second polymer is PLA. In some embodiments, the hydrophobic portion of the second polymer is PGA. In some embodiments, the hydrophobic portion of the second polymer is a copolymer of lactic and glycolic acid (e.g., PLGA). In some embodiments, the hydrophobic portion of the second polymer has a weight average molecular weight of from about 1 kDa to about 20 kDa (e.g., from about 1 kDa to about 18 kDa, 17 kDa, 16 kDa, 15 kDa, 14 kDa or 13 kDa, from about 2 kDa to about 12 kDa, from about 6 kDa to about 20 kDa, from about 5 kDa to about 18 kDa, from about 7 kDa to about 17 kDa, from about 8 kDa to about 13 kDa, from about 9 kDa to about 11 kDa, from about 10 kDa to about 14 kDa, from about 6 kDa to about 8 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa or about 17 kDa).

In some embodiments, the hydrophilic polymer portion of the second polymer is PEG. In some embodiments, the hydrophilic portion of the second polymer has a weight average molecular weight of from about 1 kDa to about 21 kDa (e.g., from about 1 kDa to about 3 kDa, e.g., about 2 kDa, or from about 2 kDa to about 5 kDa, e.g., about 3.5 kDa, or from about 4 kDa to about 6 kDa, e.g., about 5 kDa). In some embodiments, the ratio of weight average molecular weight of the hydrophilic to hydrophobic polymer portions of the second polymer is from about 1:1 to about 1:20 (e.g., about 1:4 to about 1:10, about 1:4 to about 1:7, about 1:3 to about 1:7, about 1:3 to about 1:6, about 1:4 to about 1:6.5 (e.g., 1:4, 1:4.5, 1:5, 1:5.5, 1:6, 1:6.5) or about 1:1 to about 1:4 (e.g., about 1:1.4, 1:1.8, 1:2, 1:2.4, 1:2.8, 1:3, 1:3.2, 1:3.5 or 1:4). In one embodiment, the hydrophilic portion of the second polymer has a weight average molecular weight of from about 2 kDa to 3.5 kDa and the ratio of the weight average molecular weight of the hydrophilic to hydrophobic portions of the second polymer is from about 1:4 to about 1:6.5 (e.g., 1:4, 1:4.5, 1:5, 1:5.5, 1:6, 1:6.5). In one embodiment, the hydrophilic portion of the second polymer has a weight average molecular weight of from about 4 kDa to 6 kDa (e.g., 5 kDa) and the ratio of the weight average molecular weight of the hydrophilic to hydrophobic portions of the second polymer is from about 1:1 to about 1:3.5 (e.g., about 1:1.4, 1:1.8, 1:2, 1:2.4, 1:2.8, 1:3, 1:3.2, or 1:3.5).

In some embodiments, the hydrophilic polymer portion of the second polymer has a terminal hydroxyl moiety. In some embodiments, the hydrophilic polymer portion of the second polymer has a terminal alkoxy moiety. In some embodiments, the hydrophilic polymer portion of the second polymer is a methoxy PEG (e.g., a terminal methoxy PEG). In some embodiments, the hydrophilic polymer portion of the second polymer does not have a terminal alkoxy moiety. In some embodiments, the terminus of the hydrophilic polymer portion of the second polymer is conjugated to a hydrophobic polymer, e.g., to make a triblock copolymer.

In some embodiments, the hydrophilic polymer portion of the second polymer comprises a terminal conjugate. In some embodiments, the terminal conjugate is a targeting agent or a dye. In some embodiments, the terminal conjugate is a folate or a rhodamine. In some embodiments, the terminal conjugate is a targeting peptide (e.g., an RGD peptide).

In some embodiments, the hydrophilic polymer portion of the second polymer is attached to the hydrophobic polymer portion through a covalent bond. In some embodiments, the hydrophilic polymer is attached to the hydrophobic polymer through an amide, ester, ether, amino, carbamate, or carbonate bond (e.g., an ester or an amide).

In some embodiments, the ratio of the first and second polymer is from about 1:1 to about 20:1, e.g., about 1:1 to about 10:1, e.g., about 1:1 to 9:1, or about 1.2: to 8:1. In some embodiments, the ratio of the first and second polymer is from about 85:15 to about 55:45 percent by weight or about 84:16 to about 60:40 percent by weight.

In some embodiments the particle is substantially free of a targeting agent (e.g., of a targeting agent covalently linked to a component of the particle, e.g., to the first or second polymer or agent), e.g., a targeting agent able to bind to or otherwise associate with a target biological entity, e.g., a membrane component, a cell surface receptor, prostate specific membrane antigen, or the like. In some embodiments the particle is substantially free of a targeting agent that causes the particle to become localized to a tumor, a disease site, a tissue, an organ, a type of cell, e.g., a cancer cell, within the body of a subject to whom a therapeutically effective amount of the particle is administered. In some embodiments, the particle is substantially free of a targeting agent selected from nucleic acid aptamers, growth factors, hormones, cytokines, interleukins, antibodies, integrins, fibronectin receptors, p-glycoprotein receptors, peptides and cell binding sequences. In some embodiments, no polymer is conjugated to a targeting moiety. In an embodiment substantially free of a targeting agent means substantially free of any moiety other than the first polymer, the second polymer, a third polymer (if present), a surfactant (if present), and the agent, e.g., an epothilone or anti-cancer agent, that targets the particle. Thus, in such embodiments, any contribution to localization by the first polymer, the second polymer, a third polymer (if present), a surfactant (if present), and the agent is not considered to be “targeting.” In an embodiment the particle is free of moieties added for the purpose of selectively targeting the particle to a site in a subject, e.g., by the use of a moiety on the particle having a high and specific affinity for a target in the subject.

In some embodiments the second polymer is other than a lipid, e.g., other than a phospholipid. In some embodiments the particle is substantially free of an amphiphilic layer that reduces water penetration into the nanoparticle. In some embodiment the particle comprises less than 5 or 10% (e.g., as determined as w/w, v/v) of a lipid, e.g., a phospholipid. In some embodiments the particle is substantially free of a lipid layer, e.g., a phospholipid layer, e.g., that reduces water penetration into the nanoparticle. In some embodiments the particle is substantially free of lipid, e.g., is substantially free of phospholipid.

In some embodiments the particle is substantially free of a radiopharmaceutical agent, e.g., a radiotherapeutic agent, radiodiagnostic agent, prophylactic agent, or other radioisotope. In some embodiments the particle is substantially free of an immunomodulatory agent, e.g., an immunostimulatory agent or immunosuppressive agent. In some embodiments the particle is substantially free of a vaccine or immunogen, e.g., a peptide, sugar, lipid-based immunogen, B cell antigen or T cell antigen. In some embodiments, the particle is substantially free of water soluble PLGA (e.g., PLGA having a weight average molecular weight of less than about 1 kDa).

In some embodiments, the ratio of the first polymer to the second polymer is such that the particle comprises at least 5%, 8%, 10%, 12%, 15%, 18%, 20%, 23%, 25%, or 30% by weight of a polymer having a hydrophobic portion and a hydrophilic portion.

In some embodiments, the zeta potential of the particle surface, when measured in water, is from about −80 mV to about 50 mV, e.g., about −50 mV to about 30 mV, about −20 mV to about 20 mV, or about −10 mV to about 10 mV. In some embodiments, the zeta potential of the particle surface, when measured in water, is neutral or slightly negative. In some embodiments, the zeta potential of the particle surface, when measured in water, is less than 0, e.g., about 0 mV to about −20 mV.

In some embodiments, the particle comprises less than 5000 ppm of a solvent (e.g., acetone, tert-butylmethyl ether, heptane, dichloromethane, dimethylformamide, ethyl acetate, acetonitrile, tetrahydrofuran, ethanol, methanol, isopropyl alcohol, methyl ethyl ketone, butyl acetate, or propyl acetate), e.g., less than 4500 ppm, less than 4000 ppm, less than 3500 ppm, less than 3000 ppm, less than 2500 ppm, less than 2000 ppm, less than 1500 ppm, less than 1000 ppm, less than 500 ppm, less than 250 ppm, less than 100 ppm, less than 50 ppm, less than 25 ppm, less than 10 ppm, less than 5 ppm, less than 2 ppm, or less than 1 ppm). In some embodiments, the particle is substantially free of a solvent (e.g., acetone, tert-butylmethyl ether, heptane, dichloromethane, dimethylformamide, ethyl acetate, acetonitrile, tetrahydrofuran, ethanol, methanol, isopropyl alcohol, methyl ethyl ketone, butyl acetate, or propyl acetate).

In some embodiments, the particle is substantially free of a class II or class III solvent as defined by the United States Department of Health and Human Services Food and Drug Administration “Q3c-Tables and List.” In some embodiments, the particle comprises less than 5000 ppm of acetone. In some embodiments, the particle comprises less than 5000 ppm of tert-butylmethyl ether. In some embodiments, the particle comprises less than 5000 ppm of heptane. In some embodiments, the particle comprises less than 600 ppm of dichloromethane. In some embodiments, the particle comprises less than 880 ppm of dimethylformamide. In some embodiments, the particle comprises less than 5000 ppm of ethyl acetate. In some embodiments, the particle comprises less than 410 ppm of acetonitrile. In some embodiments, the particle comprises less than 720 ppm of tetrahydrofuran. In some embodiments, the particle comprises less than 5000 ppm of ethanol. In some embodiments, the particle comprises less than 3000 ppm of methanol. In some embodiments, the particle comprises less than 5000 ppm of isopropyl alcohol. In some embodiments, the particle comprises less than 5000 ppm of methyl ethyl ketone. In some embodiments, the particle comprises less than 5000 ppm of butyl acetate. In some embodiments, the particle comprises less than 5000 ppm of propyl acetate.

In some embodiments, a composition comprising a plurality of particles is substantially free of solvent.

In some embodiments, in a composition of a plurality of particles, the particles have an average diameter of from about 50 to about 500 nm (e.g., from about 50 to about 200 nm). In some embodiments, in a composition of a plurality of particles, the particles have a Dv50 (median particle size) from about 50 nm to about 220 nm (e.g., from about 75 nm to about 200 nm). In some embodiments, in a composition of a plurality of particles, the particles have a Dv90 (particle size below which 90% of the volume of particles exists) of about 50 nm to about 500 nm (e.g., about 75 nm to about 220 nm).

In some embodiments, the agent is in the form of a salt (e.g., an insoluble salt). In some embodiments, the agent is in the form of a prodrug (i.e., the prodrug releases the agent in vivo).

In some embodiments, the agent is an epothilone selected from ixabepilone, epothilone B, epothilone D, BMS310705, dehydelone and ZK-EPO. In some embodiments, the agent is an epothilone described herein.

In some embodiments, the agent is present in the particle in an amount of from about 1 to about 30% by weight (e.g., from about 3 to about 30% by weight, from about 4 to about 25% by weight, or from about 5 to about 13%, 14%, 15%, 16%, 17%, 18%, 19% or 20% by weight).

In some embodiments, at least about 50% of the agent is embedded in the particle (e.g., embedded in the first polymer and/or the second polymer). In some embodiments, substantially all of the agent is embedded in particle (e.g., embedded in the first polymer and/or the second polymer).

In an embodiment the particle comprises the enumerated elements.

In an embodiment the particle consists of the enumerated elements.

In an embodiment the particle consists essentially of the enumerated elements.

In another aspect, the invention features a particle. The particle comprises:

a first polymer and a second polymer;

a first agent and a second agent, wherein the first agent is attached to the first polymer to form a first polymer-agent conjugate, and the second agent is attached to the second polymer to form a second polymer-agent conjugate; and

a third polymer, the third polymer comprising a hydrophilic portion and a hydrophobic portion,

wherein at least one of the first or second agent is an epothilone.

In some embodiments, the epothilone is selected from ixabepilone, epothilone B, epothilone D, BMS310705, dehydelone and ZK-EPO. In some embodiments, the epothilone is an epothilone described herein. In some embodiments, at least one of the first or second agent is an epothilone, and the other of the first or second agent is an anti-cancer agent, e.g., an anti-cancer agent described herein. In some embodiments, the anti-cancer agent is an agent other than an epothilone.

In some embodiments, the first and second agent have the same chemical structure. In some embodiments, the first agent and second agent have the same chemical structure and are attached to the respective polymers via the same point of attachment. In some embodiments, the first agent and second agent have the same chemical structure and are attached to the respective polymers through different points of attachment. In some embodiments, the first and second agent have different chemical structures.

In some embodiments, the particle is a nanoparticle. In some embodiments, the nanoparticle has a diameter of less than or equal to about 220 nm (e.g., less than or equal to about 215 nm, 210 nm, 205 nm, 200 nm, 195 nm, 190 nm, 185 nm, 180 nm, 175 nm, 170 nm, 165 nm, 160 nm, 155 nm, 150 nm, 145 nm, 140 nm, 135 nm, 130 nm, 125 nm, 120 nm, 115 nm, 110 nm, 105 nm, 100 nm, 95 nm, 90 nm, 85 nm, 80 nm, 75 nm, 70 nm, 65 nm, 60 nm, 55 nm or 50 nm).

In some embodiments, the first polymer is a PLGA polymer. In some embodiments, the second polymer is a PLGA polymer. In some embodiments, both the first and second polymers are PLGA polymers.

In some embodiments, the particle has one or more of the following properties:

it further comprises a compound comprising at least one acidic moiety,

wherein the compound is a polymer or a small molecule;

it further comprises a surfactant;

the first or second polymer is a PLGA polymer, wherein the ratio of lactic acid to glycolic acid is from about 25:75 to about 75:25;

the first or second polymer is a PLGA polymer, and the weight average molecular weight of the first polymer is from about 1 to about 20 kDa, e.g., is about 1, 2, 3, 4, 5, 6, 7, 8, 9, 10, 11, 12, 13, 14, 15, 16, 17, 18, 19 or 20 kDa; or the ratio of the combined first and second polymer to the third polymer is such that the particle comprises at least 5%, 10%, 15%, 20%, 25% by weight of a polymer having a hydrophobic portion and a hydrophilic portion.

In an embodiment the first agent is attached to a first polymer, the second agent is attached to a second polymer and:

the first and second agents are the same, e.g., the same epothilone;

the first and second agents are the same, e.g., the same epothilone, and the first and second polymers are different from one another. E.g., the first and second polymers differ by molecular weight, subunit composition (e.g., the first and second polymers are PLGA polymers having different ratios of ratio of lactic acid monomers to glycolic acid monomers), or subunit identity, e.g. a chitosan polymer and a PLGA polymer;

the first and second agents are different agents, e.g., two different epothilones, or one epothilone and one anti-cancer agent;

the first and second agents are different agents, e.g., two different epothilones, or one epothilone and one anti-cancer agent, and the first and second polymers have the same structure, e.g., they are the same PLGA polymer; or

the first and second agents are different agents, e.g., two different epothilones, or one epothilone and one anti-cancer agent, and the first and second polymers are different from one another. E.g., the first and second polymers differ by molecular weight, subunit composition (e.g., the first and second polymers are PLGA polymers having different ratios of ratio of lactic acid monomers to glycolic acid monomers), or subunit identity, e.g. a chitosan polymer and a PLGA polymer.

In an embodiment the first agent is released from the first polymer-agent conjugate with a first release profile and the second agent is released from the second polymer-agent conjugate with a second release profile. E.g., a bond between the first agent and the first polymer is more rapidly broken than a bond between the second agent and the second polymer. E.g., the first polymer-agent conjugate can comprise a first linker (e.g., a linker or a bond) linking the first agent to the first polymer and the second polymer-agent conjugate can comprise a second linker (e.g., a linker or a bond) linking the second agent to the second polymer, wherein the linkers provide for different profiles for release of the first and second agents from their respective agent-polymer conjugates. As described above, the first and second agents can differ or be the same. Similarly, the first and second polymers can differ or be the same. Thus, the release profile of one or more agents can be optimized.

In some embodiments, the particle further comprises a compound comprising at least one acidic moiety, wherein the compound is a polymer or a small molecule.

In some embodiments, the compound comprising at least one acidic moiety is a polymer comprising an acidic group. In some embodiments, the compound comprising at least one acidic moiety is a hydrophobic polymer. In some embodiments, the first polymer and the compound comprising at least one acidic moiety are the same polymer. In some embodiments, the compound comprising at least one acidic moiety is PLGA. In some embodiments, the ratio of lactic acid monomers to glycolic acid monomers in PLGA is from about 0.1:99.9 to about 99.9:0.1. In some embodiments, the ratio of lactic acid monomers to glycolic acid monomers in PLGA is from about 75:25 to about 25:75, e.g., about 60:40 to about 40:60 (e.g., about 50:50), about 60:40, or about 75:25. In some embodiments, the PLGA comprises a terminal hydroxyl group. In some embodiments, the PLGA comprises a terminal acyl group (e.g., an acetyl group).

In some embodiments, the weight average molecular weight of the compound comprising at least one acidic moiety is from about 1 kDa to about 20 kDa (e.g., from about 1 kDa to about 15 kDa, from about 2 kDa to about 12 kDa, from about 6 kDa to about 20 kDa, from about 5 kDa to about 15 kDa, from about 7 kDa to about 11 kDa, from about 5 kDa to about 10 kDa, from about 7 kDa to about 10 kDa, from about 5 kDa to about 7 kDa, from about 6 kDa to about 8 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa or about 17 kDa). In some embodiments, the compound comprising at least one acidic moiety has a glass transition temperature of from about 20° C. to about 60° C.

In some embodiments, the compound comprising at least one acidic moiety has a polymer polydispersity index of less than or equal to about 2.5 (e.g., less than or equal to about 2.2, or less than or equal to about 2.0). In some embodiments, the compound comprising at least one acidic moiety has a polymer polydispersity index of about 1.0 to about 2.5, e.g., from about 1.0 to about 2.0, from about 1.0 to about 1.8, from about 1.0 to about 1.7, or from about 1.0 to about 1.6.

In some embodiments, the particle comprises a plurality of compounds comprising at least one acidic moiety. For example, in some embodiments, one compound of the plurality of compounds comprising at least one acidic moiety is a PLGA polymer wherein the hydroxy terminus is functionalized with an acetyl group, and another compound in the plurality is a PLGA polymer wherein the hydroxy terminus is unfunctionalized.

In some embodiments, the percent by weight of the compound comprising at least one acidic moiety within the particle is up to about 50% (e.g., up to about 45% by weight, up to about 40% by weight, up to about 35% by weight, up to about 30% by weight, from about 0 to about 30% by weight, e.g., about 4.5%, about 9%, about 12%, about 15%, about 18%, about 20%, about 22%, about 24%, about 26%, about 28% or about 30%).

In some embodiments, the compound comprising at least one acidic moiety is a small molecule comprising an acidic group.

In some embodiments, the particle further comprises a surfactant. In some embodiments, the surfactant is PEG, PVA, PVP, poloxamer, a polysorbate, a polyoxyethylene ester, a PEG-lipid (e.g., PEG-ceramide, d-alpha-tocopheryl polyethylene glycol 1000 succinate), 1,2-Distearoyl-sn-Glycero-3-[Phospho-rac-(1-glycerol)] or lecithin. In some embodiments, the surfactant is PVA and the PVA is from about 3 kDa to about 50 kDa (e.g., from about 5 kDa to about 45 kDa, about 7 kDa to about 42 kDa, from about 9 kDa to about 30 kDa, or from about 11 to about 28 kDa) and up to about 98% hydrolyzed (e.g., about 75-95%, about 80-90% hydrolyzed, or about 85% hydrolyzed). In some embodiments, the surfactant is polysorbate 80. In some embodiments, the surfactant is Solutol® HS 15. In some embodiments, the surfactant is present in an amount of up to about 35% by weight of the particle (e.g., up to about 20% by weight or up to about 25% by weight, from about 15% to about 35% by weight, from about 20% to about 30% by weight, or from about 23% to about 26% by weight).

In some embodiments, the particle further comprises a stabilizer or lyoprotectant, e.g., a stabilizer or lyoprotectant described herein. In some embodiments, the stabilizer or lyoprotectant is a carbohydrate (e.g., a carbohydrate described herein, such as, e.g., sucrose, cyclodextrin or a derivative of cyclodextrin (e.g. 2-hydroxypropyl-β-cyclodextrin)), salt, PEG, PVP or crown ether.

In an embodiment the amount of first and second agent in the particle that is not attached to the first or second polymer is less than about 5% (e.g., less than about 2% or less than about 1%, e.g., in terms of w/w or number/number) of the amount of first or second agent attached to the first polymer or second polymer.

In some embodiments, the first polymer is a biodegradable polymer (e.g., PLA, PGA, PLGA, PCL, PDO, polyanhydrides, polyorthoesters, or chitosan). In some embodiments, the first polymer is a hydrophobic polymer. In some embodiments, the percent by weight of the first polymer within the particle is from about 20% to about 90% (e.g., from about 20% to about 80%, from about 25% to about 75%, or from about 30% to about 70%). In some embodiments, the first polymer is PLA. In some embodiments, the first polymer is PGA.

In some embodiments, the first polymer is a copolymer of lactic and glycolic acid (e.g., PLGA). In some embodiments, the first polymer is a PLGA-ester. In some embodiments, the first polymer is a PLGA-lauryl ester. In some embodiments, the first polymer comprises a terminal free acid. In some embodiments, the first polymer comprises a terminal acyl group (e.g., an acetyl group). In some embodiments, the polymer comprises a terminal hydroxyl group. In some embodiments, the ratio of lactic acid monomers to glycolic acid monomers in PLGA is from about 0.1:99.9 to about 99.9:0.1. In some embodiments, the ratio of lactic acid monomers to glycolic acid monomers in PLGA is from about 75:25 to about 25:75, e.g., about 60:40 to about 40:60 (e.g., about 50:50), about 60:40, or about 75:25.

In some embodiments, the weight average molecular weight of the first polymer is from about 1 kDa to about 20 kDa (e.g., from about 1 kDa to about 15 kDa, from about 2 kDa to about 12 kDa, from about 6 kDa to about 20 kDa, from about 5 kDa to about 15 kDa, from about 7 kDa to about 11 kDa, from about 5 kDa to about 10 kDa, from about 7 kDa to about 10 kDa, from about 5 kDa to about 7 kDa, from about 6 kDa to about 8 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa or about 17 kDa). In some embodiments, the first polymer has a glass transition temperature of from about 20° C. to about 60° C. In some embodiments, the first polymer has a polymer polydispersity index of less than or equal to about 2.5 (e.g., less than or equal to about 2.2, or less than or equal to about 2.0). In some embodiments, the first polymer has a polymer polydispersity index of about 1.0 to about 2.5, e.g., from about 1.0 to about 2.0, from about 1.0 to about 1.8, from about 1.0 to about 1.7, or from about 1.0 to about 1.6.

In some embodiments, the second polymer is a biodegradable polymer (e.g., PLA, PGA, PLGA, PCL, PDO, polyanhydrides, polyorthoesters, or chitosan). In some embodiments, the second polymer is a hydrophobic polymer. In some embodiments, the percent by weight of the second polymer within the particle is from about 20% to about 90% (e.g., from about 20% to about 80%, from about 25% to about 75%, or from about 30% to about 70%). In some embodiments, the second polymer is PLA. In some embodiments, the second polymer is PGA.

In some embodiments, the second polymer is a copolymer of lactic and glycolic acid (e.g., PLGA). In some embodiments, the second polymer is a PLGA-ester. In some embodiments, the second polymer is a PLGA-lauryl ester. In some embodiments, the second polymer comprises a terminal free acid. In some embodiments, the second polymer comprises a terminal acyl group (e.g., an acetyl group). In some embodiments, the polymer comprises a terminal hydroxyl group. In some embodiments, the ratio of lactic acid monomers to glycolic acid monomers in PLGA is from about 0.1:99.9 to about 99.9:0.1. In some embodiments, the ratio of lactic acid monomers in PLGA to glycolic acid monomers is from about 75:25 to about 25:75, e.g., about 60:40 to about 40:60 (e.g., about 50:50), about 60:40, or about 75:25.

In some embodiments, the weight average molecular weight of the second polymer is from about 1 kDa to about 20 kDa (e.g., from about 1 kDa to about 15 kDa, from about 2 kDa to about 12 kDa, from about 6 kDa to about 20 kDa, from about 5 kDa to about 15 kDa, from about 7 kDa to about 11 kDa, from about 5 kDa to about 10 kDa, from about 7 kDa to about 10 kDa, from about 5 kDa to about 7 kDa, from about 6 kDa to about 8 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa or about 17 kDa). In some embodiments, the second polymer has a glass transition temperature of from about 20° C. to about 60° C. In some embodiments, the second polymer has a polymer polydispersity index of less than or equal to about 2.5 (e.g., less than or equal to about 2.2, or less than or equal to about 2.0). In some embodiments, the second polymer has a polymer polydispersity index of about 1.0 to about 2.5, e.g., from about 1.0 to about 2.0, from about 1.0 to about 1.8, from about 1.0 to about 1.7, or from about 1.0 to about 1.6.

In some embodiments, the percent by weight of the third polymer within the particle is up to about 50% by weight (e.g., from about 4 to any of about 50%, about 5%, about 10%, about 15%, about 20%, about 25%, about 30%, about 35%, about 40%, about 45% or about 50% by weight). In some embodiments, the third polymer has a hydrophilic portion and a hydrophobic portion. In some embodiments, the third polymer is a block copolymer. In some embodiments, the third polymer comprises two regions, the two regions together being at least about 70% by weight of the polymer (e.g., at least about 80%, at least about 90%, at least about 95%). In some embodiments, the third polymer is a block copolymer comprising a hydrophobic polymer and a hydrophilic polymer. In some embodiments, the third polymer, e.g., a diblock copolymer, comprises a hydrophobic polymer and a hydrophilic polymer. In some embodiments, the third polymer, e.g., a triblock copolymer, comprises a hydrophobic polymer, a hydrophilic polymer and a hydrophobic polymer, e.g., PLA-PEG-PLA, PGA-PEG-PGA, PLGA-PEG-PLGA, PCL-PEG-PCL, PDO-PEG-PDO, PEG-PLGA-PEG, PLA-PEG-PGA, PGA-PEG-PLA, PLGA-PEG-PLA or PGA-PEG-PLGA.

In some embodiments, the hydrophobic portion of the third polymer is a biodegradable polymer (e.g., PLA, PGA, PLGA, PCL, PDO, polyanhydrides, polyorthoesters, or chitosan). In some embodiments, the hydrophobic portion of the third polymer is PLA. In some embodiments, the hydrophobic portion of the third polymer is PGA. In some embodiments, the hydrophobic portion of the third polymer is a copolymer of lactic and glycolic acid (e.g., PLGA). In some embodiments, the hydrophobic portion of the third polymer has a weight average molecular weight of from about 1 kDa to about 20 kDa (e.g., from about 1 kDa to about 18 kDa, 17 kDa, 16 kDa, 15 kDa, 14 kDa or 13 kDa, from about 2 kDa to about 12 kDa, from about 6 kDa to about 20 kDa, from about 5 kDa to about 18 kDa, from about 7 kDa to about 17 kDa, from about 8 kDa to about 13 kDa, from about 9 kDa to about 11 kDa, from about 10 kDa to about 14 kDa, from about 6 kDa to about 8 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa or about 17 kDa).

In some embodiments, the hydrophilic polymer portion of the third polymer is PEG. In some embodiments, the hydrophilic portion of the third polymer has a weight average molecular weight of from about 1 kDa to about 21 kDa (e.g., from about 1 kDa to about 3 kDa, e.g., about 2 kDa, or from about 2 kDa to about 5 kDa, e.g., about 3.5 kDa, or from about 4 kDa to about 6 kDa, e.g., about 5 kDa). In some embodiments, the ratio of weight average molecular weight of the hydrophilic to hydrophobic polymer portions of the third polymer is from about 1:1 to about 1:20 (e.g., about 1:4 to about 1:10, about 1:4 to about 1:7, about 1:3 to about 1:7, about 1:3 to about 1:6, about 1:4 to about 1:6.5 (e.g., 1:4, 1:4.5, 1:5, 1:5.5, 1:6, 1:6.5) or about 1:1 to about 1:4 (e.g., about 1:1.4, 1:1.8, 1:2, 1:2.4, 1:2.8, 1:3, 1:3.2, 1:3.5 or 1:4). In one embodiment, the hydrophilic portion of the third polymer has a weight average molecular weight of from about 2 kDa to 3.5 kDa and the ratio of the weight average molecular weight of the hydrophilic to hydrophobic portions of the third polymer is from about 1:4 to about 1:6.5 (e.g., 1:4, 1:4.5, 1:5, 1:5.5, 1:6, 1:6.5). In one embodiment, the hydrophilic portion of the third polymer has a weight average molecular weight of from about 4 kDa to 6 kDa (e.g., 5 kDa) and the ratio of the weight average molecular weight of the hydrophilic to hydrophobic portions of the third polymer is from about 1:1 to about 1:3.5 (e.g., about 1:1.4, 1:1.8, 1:2, 1:2.4, 1:2.8, 1:3, 1:3.2, or 1:3.5).

In some embodiments, the hydrophilic polymer portion of the third polymer has a terminal hydroxyl moiety. In some embodiments, the hydrophilic polymer portion of the third polymer has a terminal alkoxy moiety. In some embodiments, the hydrophilic polymer portion of the third polymer is a methoxy PEG (e.g., a terminal methoxy PEG). In some embodiments, the hydrophilic polymer portion of the third polymer does not have a terminal alkoxy moiety. In some embodiments, the terminus of the hydrophilic polymer portion of the third polymer is conjugated to hydrophobic polymer, e.g., to make a triblock copolymer.

In some embodiments, the hydrophilic polymer portion of the third polymer comprises a terminal conjugate. In some embodiments, the terminal conjugate is a targeting agent or a dye. In some embodiments, the terminal conjugate is a folate or a rhodamine. In some embodiments, the terminal conjugate is a targeting peptide (e.g., an RGD peptide).

In some embodiments, the hydrophilic polymer portion of the third polymer is attached to the hydrophobic polymer portion through a covalent bond. In some embodiments, the hydrophilic polymer is attached to the hydrophobic polymer through an amide, ester, ether, amino, carbamate, or carbonate bond (e.g., an ester or an amide).

In some embodiments, the ratio by weight of the combined first and second polymers to the third polymer is from about 1:1 to about 20:1, e.g., about 1:1 to about 10:1, e.g., about 1:1 to 9:1, or about 1.2: to 8:1. In some embodiments, the ratio of the first and second polymer is from about 85:15 to about 55:45 percent by weight or about 84:16 to about 60:40 percent by weight. In some embodiments, the ratio by weight of the combined first and second polymers to the compound comprising at least one acidic moiety is from about 1:3 to about 1000:1, e.g., about 1:1 to about 10:1, or about 1.5:1. In some embodiments, the ratio of the third polymer to the compound comprising at least one acidic moiety is from about 1:10 to about 250:1, e.g., from about 1:5 to about 5:1, or from about 1:3.5 to about 1:1.

In some embodiments the particle is substantially free of a targeting agent (e.g., of a targeting agent covalently linked to a component of the particle, e.g., to the first or second polymer or agent), e.g., a targeting agent able to bind to or otherwise associate with a target biological entity, e.g., a membrane component, a cell surface receptor, prostate specific membrane antigen, or the like. In some embodiments the particle is substantially free of a targeting agent that causes the particle to become localized to a tumor, a disease site, a tissue, an organ, a type of cell, e.g., a cancer cell, within the body of a subject to whom a therapeutically effective amount of the particle is administered. In some embodiments, the particle is substantially free of a targeting agent selected from nucleic acid aptamers, growth factors, hormones, cytokines, interleukins, antibodies, integrins, fibronectin receptors, p-glycoprotein receptors, peptides and cell binding sequences. In some embodiments, no polymer is conjugated to a targeting moiety. In an embodiment substantially free of a targeting agent means substantially free of any moiety other than the first polymer, the second polymer, a third polymer (if present), a surfactant (if present), and the agent, e.g., an epothilone or anti-cancer agent, that targets the particle. Thus, in such embodiments, any contribution to localization by the first polymer, the second polymer, a third polymer (if present), a surfactant (if present), and the agent is not considered to be “targeting.” In an embodiment the particle is free of moieties added for the purpose of selectively targeting the particle to a site in a subject, e.g., by the use of a moiety on the particle having a high and specific affinity for a target in the subject.

In some embodiments the third polymer is other than a lipid, e.g., other than a phospholipid. In some embodiments the particle is substantially free of an amphiphilic layer that reduces water penetration into the nanoparticle. In some embodiment the particle comprises less than 5 or 10% (e.g., as determined as w/w, v/v) of a lipid, e.g., a phospholipid. In some embodiments the particle is substantially free of a lipid layer, e.g., a phospholipid layer, e.g., that reduces water penetration into the nanoparticle. In some embodiments the particle is substantially free of lipid, e.g., is substantially free of phospholipid.

In some embodiments the particle is substantially free of a radiopharmaceutical agent, e.g., a radiotherapeutic agent, radiodiagnostic agent, prophylactic agent, or other radioisotope. In some embodiments the particle is substantially free of an immunomodulatory agent, e.g., an immunostimulatory agent or immunosuppressive agent. In some embodiments the particle is substantially free of a vaccine or immunogen, e.g., a peptide, sugar, lipid-based immunogen, B cell antigen or T cell antigen. In some embodiments, the particle is substantially free of water soluble PLGA (e.g., PLGA having a weight average molecular weight of less than about 1 kDa).

In some embodiments, the ratio of the combined first and second polymer to the third polymer is such that the particle comprises at least 5%, 8%, 10%, 12%, 15%, 18%, 20%, 23%, 25% or 30% by weight of a polymer having a hydrophobic portion and a hydrophilic portion.

In some embodiments, the zeta potential of the particle surface, when measured in water, is from about −80 mV to about 50 mV, e.g., about −50 mV to about 30 mV, about −20 mV to about 20 mV, or about −10 mV to about 10 mV. In some embodiments, the zeta potential of the particle surface, when measured in water, is neutral or slightly negative. In some embodiments, the zeta potential of the particle surface, when measured in water, is less than 0, e.g., about 0 mV to about −20 mV.

In some embodiments, the particle comprises less than 5000 ppm of a solvent (e.g., acetone, tert-butylmethyl ether, heptane, dichloromethane, dimethylformamide, ethyl acetate, acetonitrile, tetrahydrofuran, ethanol, methanol, isopropyl alcohol, methyl ethyl ketone, butyl acetate, or propyl acetate), (e.g., less than 4500 ppm, less than 4000 ppm, less than 3500 ppm, less than 3000 ppm, less than 2500 ppm, less than 2000 ppm, less than 1500 ppm, less than 1000 ppm, less than 500 ppm, less than 250 ppm, less than 100 ppm, less than 50 ppm, less than 25 ppm, less than 10 ppm, less than 5 ppm, less than 2 ppm, or less than 1 ppm). In some embodiments, the particle is substantially free of a solvent (e.g., acetone, tert-butylmethyl ether, heptane, dichloromethane, dimethylformamide, ethyl acetate, acetonitrile, tetrahydrofuran, ethanol, methanol, isopropyl alcohol, methyl ethyl ketone, butyl acetate, or propyl acetate).

In some embodiments, the particle is substantially free of a class II or class III solvent as defined by the United States Department of Health and Human Services Food and Drug Administration “Q3c-Tables and List.” In some embodiments, the particle comprises less than 5000 ppm of acetone. In some embodiments, the particle comprises less than 5000 ppm of tert-butylmethyl ether. In some embodiments, the particle comprises less than 5000 ppm of heptane. In some embodiments, the particle comprises less than 600 ppm of dichloromethane. In some embodiments, the particle comprises less than 880 ppm of dimethylformamide. In some embodiments, the particle comprises less than 5000 ppm of ethyl acetate. In some embodiments, the particle comprises less than 410 ppm of acetonitrile. In some embodiments, the particle comprises less than 720 ppm of tetrahydrofuran. In some embodiments, the particle comprises less than 5000 ppm of ethanol. In some embodiments, the particle comprises less than 3000 ppm of methanol. In some embodiments, the particle comprises less than 5000 ppm of isopropyl alcohol. In some embodiments, the particle comprises less than 5000 ppm of methyl ethyl ketone. In some embodiments, the particle comprises less than 5000 ppm of butyl acetate. In some embodiments, the particle comprises less than 5000 ppm of propyl acetate.

In some embodiments, a composition comprising a plurality of particles is substantially free of solvent.

In some embodiments, in a composition of a plurality of particles, the particles have an average diameter of from about 50 nm to about 500 nm (e.g., from about 50 to about 200 nm). In some embodiments, in a composition of a plurality of particles, the particles have a Dv50 (median particle size) from about 50 nm to about 220 nm (e.g., from about 75 nm to about 200 nm). In some embodiments, in a composition of a plurality of particles, the particles have a Dv90 (particle size below which 90% of the volume of particles exists) of about 50 nm to about 500 nm (e.g., about 75 nm to about 220 nm).

In some embodiments, a single first agent is attached to a single first polymer, e.g., to a terminal end of the polymer. In some embodiments, a plurality of first agents are attached to a single first polymer (e.g., 2, 3, 4, 5, 6, or more). In some embodiments, the agents are the same agent. In some embodiments, the agents are different agents. In some embodiments, a single second agent is attached to a single second polymer, e.g., to a terminal end of the polymer. In some embodiments, a plurality of second agents are attached to a single second polymer (e.g., 2, 3, 4, 5, 6, or more). In some embodiments, the agents are the same agent. In some embodiments, the agents are different agents.

In some embodiments, the first agent is an epothilone is selected from ixabepilone, epothilone B, epothilone D, BMS310705, dehydelone and ZK-EPO. In some embodiments, the epothilone is an epothilone described herein. In some embodiments, the first agent is an anti-cancer agent.

In some embodiments, the second agent is an epothilone is selected from ixabepilone, epothilone B, epothilone D, BMS310705, dehydelone and ZK-EPO. In some embodiments, the epothilone is an epothilone described herein. In some embodiments, the second agent is an anti-cancer agent.

In some embodiments, the first agent is an epothilone attached to the first polymer via the hydroxyl group at the 3 position. In some embodiments, the first agent is an epothilone attached to the first polymer via the hydroxyl group at the 7 position.

In some embodiments, the first agent is attached directly to the first polymer, e.g., through a covalent bond. In some embodiments, the first agent is attached to a terminal end of the first polymer via an amide, ester, ether, amino, carbamate or carbonate bond. In some embodiments, the first agent is attached to a terminal end of the first polymer. In some embodiments, the first polymer comprises one or more side chains and the first agent is directly attached to the first polymer through one or more of the side chains.

In some embodiments, the second agent is an epothilone attached to the second polymer via the hydroxyl group at the 3 position. In some embodiments, the second agent is an epothilone attached to the first polymer via the hydroxyl group at the 7 position.

In some embodiments, the second agent is attached directly to the second polymer, e.g., through a covalent bond. In some embodiments, the second agent is attached to a terminal end of the second polymer via an amide, ester, ether, amino, carbamate or carbonate bond. In some embodiments, the second agent is attached to a terminal end of the second polymer. In some embodiments, the second polymer comprises one or more side chains and the second agent is directly attached to the second polymer through one or more of the side chains.

In some embodiments, the first or second polymer-agent conjugate in the particle, e.g., the nanoparticle, is:

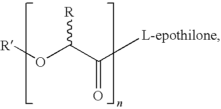

wherein L is a bond or linker, e.g., a linker described herein; and

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the epothilone is selected from ixabepilone, epothilone B, epothilone D, BMS310705, dehydelone and ZK-EPO. In some embodiments, the agent is an epothilone described herein.

In some embodiments, L is a bond.

In some embodiments, L is a linker, e.g., a linker described herein.

In some embodiments, the linker is an alkanoate linker. In some embodiments, the linker is a PEG-based linker. In some embodiments, the linker comprises a disulfide bond. In some embodiments, the linker is a self-immolative linker. In some embodiments, the linker is an amino acid or a peptide (e.g., glutamic acid such as L-glutamic acid, D-glutamic acid, DL-glutamic acid or β-glutamic acid, branched glutamic acid or polyglutamic acid). In some embodiments, the linker is β-alanine glycolate.

In some embodiments, the first or second polymer-agent conjugate in the particle, e.g., the nanoparticle, is:

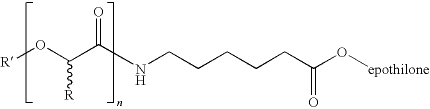

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the epothilone is selected from ixabepilone, epothilone B, epothilone D, BMS310705, dehydelone and ZK-EPO. In some embodiments, the agent is an epothilone described herein.

In some embodiments, the first or second polymer-agent conjugate in the particle, e.g., the nanoparticle, is:

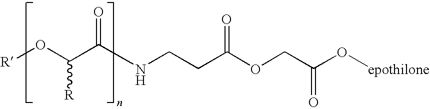

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, the epothilone is selected from ixabepilone, epothilone B, epothilone D, BMS310705, dehydelone and ZK-EPO. In some embodiments, the agent is an epothilone described herein.

In some embodiments the linker is a multifunctional linker. In some embodiments, the multifunctional linker has 2, 3, 4, 5, 6 or more reactive moieties that may be functionalized with an agent. In some embodiments, all reactive moieties are functionalized with an agent. In some embodiments, not all of the reactive moieties are functionalized with an agent (e.g., the multifunctional linker has two reactive moieties, and only one reacts with an agent; or the multifunctional linker has four reactive moieties, and only one, two or three react with an agent.)

In some embodiments, two agents are attached to a polymer via a multifunctional linker. In some embodiments, the two agents are the same agent. In some embodiments, the two agents are different agents. In some embodiments, the agent is covalently attached to the polymer via a glutamate linker.

In some embodiments, the first or second polymer-agent conjugate in the particle, e.g., the nanoparticle, is:

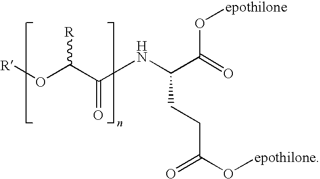

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, each epothilone is independently selected from ixabepilone, epothilone B, epothilone D, BMS310705, dehydelone and ZK-EPO. In some embodiments, each epothilone is independently selected from the epothilones described herein.

In some embodiments, at least one epothilone is attached to the polymer via the hydroxyl group at the 3 position. In some embodiments, at least one epothilone is attached to the polymer via the hydroxyl group at the 7 position. In some embodiments, each epothilone is attached via the same hydroxyl group, e.g., the hydroxyl group at the 3 position or the hydroxyl group at the 7 position. In some embodiments, each epothilone is attached via the hydroxyl group at the 3 position. In some embodiments, each epothilone is attached via the hydroxyl group at the 7 position. In some embodiments, the epothilone molecules may be attached via different hydroxyl groups, e.g., one epothilone is attached via the hydroxyl group at the 3 position and the other epothilone is attached via the hydroxyl group at the 7 position.

In some embodiments, four agents are attached to a polymer via a multifunctional linker. In some embodiments, the four agents are the same agent. In some embodiments, the four agents are different agents. In some embodiments, the agent is covalently attached to the polymer via a tri(glutamate) linker.

In some embodiments, the first or second polymer-agent conjugate in the particle, e.g., the nanoparticle, is:

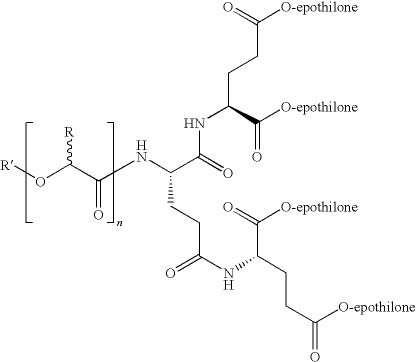

wherein about 30% to about 70%, e.g., about 35% to about 65%, 40% to about 60%, about 45% to about 55% of R substituents are hydrogen (e.g., about 50%) and about 30% to about 70%, about 35% to about 65%, about 40% to about 60%, about 45% to about 55% are methyl (e.g., about 50%); R′ is selected from hydrogen and acyl (e.g., acetyl); and wherein n is an integer from about 15 to about 308, e.g., about 77 to about 232, e.g., about 105 to about 170 (e.g., n is an integer such that the weight average molecular weight of the polymer is from about 1 kDa to about 20 kDa (e.g., from about 5 to about 15 kDa, from about 6 to about 13 kDa, or from about 7 to about 11 kDa)).

In some embodiments, each epothilone is independently selected from ixabepilone, epothilone B, epothilone D, BMS310705, dehydelone and ZK-EPO. In some embodiments, each epothilone is independently selected from the epothilones described herein.

In some embodiments, at least one epothilone is attached to the polymer via the hydroxyl group at the 3 position. In some embodiments, at least one epothilone is attached to the polymer via the hydroxyl group at the 7 position. In some embodiments, each epothilone is attached via the same hydroxyl group, e.g., the hydroxyl group at the 3 position or the hydroxyl group at the 7 position. In some embodiments, each epothilone is attached via the hydroxyl group at the 3 position. In some embodiments, each epothilone is attached via the hydroxyl group at the 7 position. In some embodiments, the epothilone molecules may be attached via different hydroxyl groups, e.g., three epothilones are attached via the hydroxyl group at the 3 position and the other epothilone is attached via the hydroxyl group at the 7 position.

In some embodiments, the particle comprises a plurality of polymer-agent conjugates. In some embodiments, the plurality of polymer-agent conjugates have the same polymer and the same agent, and differ in the nature of the linkage between the agent and the polymer. For example, in some embodiments, the polymer is PLGA, and the plurality of polymer-agent conjugates includes PLGA polymers attached to an epothilone via the hydroxyl group at the 3 position, and PLGA polymers attached to an epothilone via the hydroxyl group at the 7 position. In some embodiments, the polymer is PLGA, and the plurality of polymer-agent conjugates includes epothilone molecules attached to more than one polymer chain, e.g., epothilone molecules with PLGA polymers attached to the hydroxyl group at the 3 position and the hydroxyl group at the 7 position.

In some embodiments, the plurality of polymer-agent conjugates have the same polymer and the same agent, but the agent may be attached to the polymer via different linkers. In some embodiments, the plurality of polymer-agent conjugates includes a polymer directly attached to an agent and a polymer attached to an agent via a linker. In an embodiment, one agent is released from one polymer-agent conjugate in the plurality with a first release profile and a second agent is released from a second polymer-agent conjugate in the plurality with a second release profile. E.g., a bond between the first agent and the first polymer is more rapidly broken than a bond between the second agent and the second polymer. E.g., the first polymer-agent conjugate can comprise a first linker (e.g., a linker or a bond) linking the first agent to the first polymer and the second polymer-agent conjugate can comprise a second linker (e.g., a linker or a bond) linking the second agent to the second polymer, wherein the linkers provide for different profiles for release of the first and second agents from their respective agent-polymer conjugates.

In some embodiments, the plurality of polymer-agent conjugates includes different polymers. In some embodiments, the plurality of polymer-agent conjugates includes different agents.

In some embodiments, the first agent is present in the particle in an amount of from about 1 to about 30% by weight (e.g., from about 3 to about 30% by weight, from about 4 to about 25% by weight, or from about 5 to about 13%, 14%, 15%, 16%, 17%, 18%, 19% or 20% by weight).

In an embodiment the particle comprises the enumerated elements.

In an embodiment the particle consists of the enumerated elements.

In an embodiment the particle consists essentially of the enumerated elements.

In yet another aspect, the invention features a method of making a particle described herein, the method comprising:

providing a hydrophobic polymer having a weight average molecular weight range from about 5 kDa to about 15 kDa (e.g., about 6 to about 13 kDa, or about 7 kDa to about 11 kDa) with an agent attached thereto, wherein the agent is an epothilone,

providing a polymer comprising a hydrophilic portion and a hydrophobic portion to form a mixture, and

subjecting the mixture to conditions sufficient to form a particle comprising the agent attached to the hydrophobic polymer and the polymer having a hydrophilic portion and a hydrophobic portion.

In some embodiments, the method further comprises attaching the agent to the hydrophobic polymer.

In some embodiments, the method further comprises providing a compound comprising at least one acidic moiety in the mixture.

In some embodiments, the method further comprises providing a surfactant in the mixture.

In some embodiments, the polymer polydispersity index of the hydrophobic polymer is less than about 2.5 (e.g., less than or equal to about 2.2, or less than or equal to about 2.0). In some embodiments, the polymer has a polymer polydispersity index of about 1.0 to about 2.5, e.g., from about 1.0 to about 2.0, from about 1.0 to about 1.8, from about 1.0 to about 1.7, or from about 1.0 to about 1.6. In some embodiments, the particle is precipitated from the mixture. In some embodiments, the particle is lyophilized from the mixture.

In some embodiments, the epothilone is selected from ixabepilone, epothilone B, epothilone D, BMS310705, dehydelone and ZK-EPO. In some embodiments, the epothilone is an epothilone described herein.

In another aspect, the invention features a method of making a particle described herein, the method comprising:

providing a hydrophobic polymer having a weight average molecular weight range from about 5 kDa to about 15 kDa (e.g., about 6 to about 13 kDa, or about 7 kDa to about 11 kDa) having a first agent attached thereto,

providing a polymer comprising a hydrophilic portion and a hydrophobic portion,

providing a second agent to form a mixture, and

subjecting the mixture to conditions sufficient to form a particle comprising the first agent attached to the hydrophobic polymer, the polymer comprising a hydrophilic portion and a hydrophobic portion, and a second agent,

wherein at least one of the first or second agent is an epothilone.

In some embodiments, the epothilone is selected from ixabepilone, epothilone B, epothilone D, BMS310705, dehydelone and ZK-EPO. In some embodiments, the epothilone is an epothilone described herein. In some embodiments, at least one of the first or second agent is an epothilone, and the other of the first or second agent is an anti-cancer agent, e.g., an anti-cancer agent described herein. In some embodiments, the anti-cancer agent is an agent other than an epothilone.

In some embodiments, the method further comprises attaching the first agent to the hydrophobic polymer.

In some embodiments, the method further comprises providing a compound comprising at least one acidic moiety in the mixture.

In some embodiments, the method further comprises providing a surfactant in the mixture.

In some embodiments, the polymer polydispersity index of the hydrophobic polymer is less than about 2.5 (e.g., less than or equal to about 2.2, or less than or equal to about 2.0). In some embodiments, the polymer has a polymer polydispersity index of about 1.0 to about 2.5, e.g., from about 1.0 to about 2.0, from about 1.0 to about 1.8, from about 1.0 to about 1.7, or from about 1.0 to about 1.6. In some embodiments, the particle is precipitated from the mixture. In some embodiments, the particle is lyophilized from the mixture.

In another aspect, the invention features a method of making a particle described herein, the method comprising:

providing a hydrophobic polymer having a weight average molecular weight range from about 5 kDa to about 15 kDa (e.g., about 6 to about 13 kDa, or about 7 kDa to about 11 kDa),

providing a polymer comprising a hydrophilic portion and a hydrophobic portion,

providing an agent to form a mixture, wherein the agent is an epothilone, and

subjecting the mixture to conditions sufficient to form a particle comprising the hydrophobic polymer, the polymer comprising a hydrophilic portion and a hydrophobic portion, and the agent.

In some embodiments, the method further comprises providing a surfactant in the mixture.

In some embodiments, the epothilone is selected from ixabepilone, epothilone B, epothilone D, BMS310705, dehydelone and ZK-EPO. In some embodiments, the epothilone is an epothilone described herein.

In some embodiments, the polymer polydispersity index of the hydrophobic polymer is less than about 2.5 (e.g., less than or equal to about 2.2, or less than or equal to about 2.0). In some embodiments, the polymer has a polymer polydispersity index of about 1.0 to about 2.5, e.g., from about 1.0 to about 2.0, from about 1.0 to about 1.8, from about 1.0 to about 1.7, or from about 1.0 to about 1.6. In some embodiments, the particle is precipitated from the mixture. In some embodiments, the particle is lyophilized from of the mixture.

In another aspect, the invention features a method of making a particle described herein, the method comprising:

dissolving a hydrophobic polymer-agent conjugate and polymer comprising a hydrophilic portion and a hydrophobic portion in an organic solvent to provide an organic solution, wherein the agent is an epothilone;

combining the organic solution with an aqueous solution, the aqueous solution comprising a surfactant; and

mixing the resulting combination to provide a mixture comprising a particle described herein.

In some embodiments, the method further comprises providing a compound comprising at least one acidic moiety in the organic solution.

In some embodiments, the organic solution is filtered (e.g., through a 0.22 micron filter) prior to mixing. In some embodiments, the aqueous solution is filtered (e.g., through a 0.22 micron filter) prior to mixing.

In some embodiments, the organic solvent is miscible with water. In some embodiments, the solvent is acetone, ethanol, methanol, isopropyl alcohol, dichloromethane, acetonitrile, methyl ethyl ketone, tetrahydrofuran, butyl acetate, ethyl acetate, propyl acetate or dimethylformamide. In some embodiments, the organic solvent is immiscible with water.

In some embodiments, the ratio of the hydrophobic polymer-agent conjugate and polymer comprising a hydrophilic portion and a hydrophobic portion in the organic solution is from about 90:10 to about 55:45 weight % (e.g., from about 85:15 to about 60:40 weight %).

In some embodiments, the concentration of the surfactant in the aqueous solution is from about 0.1 to about 3.0 weight/volume. In one embodiment, the surfactant is a polymer (e.g., PVA).

In some embodiments, the mixture is purified. In some embodiments, the mixture is concentrated. In some embodiments, the mixture is subjected to tangential flow filtration or dialysis.

In some embodiments, the resulting particle is lyophilized. In one embodiment, the resulting particle is lyophilized in the presence of a lyoprotectant (e.g., a carbohydrate (e.g., a carbohydrate described herein, such as, e.g., sucrose, cyclodextrin or a derivative of cyclodextrin (e.g. 2-hydroxypropyl-β-cyclodextrin)), salt, PEG, PVP or crown ether).

In some embodiments, the method provides a plurality of particles. In one embodiment, the particles are filtered (e.g., though a 0.22 micron filter). In some embodiments, subsequent to filtering a composition of a plurality of particles, the particles have a Dv90 of less than about 200 nm.

In some embodiments, the epothilone is selected from ixabepilone, epothilone B, epothilone D, BMS310705, dehydelone and ZK-EPO. In some embodiments, the epothilone is an epothilone described herein.

In another aspect, the invention features a mixture, the mixture comprising:

a hydrophobic polymer-agent conjugate, wherein the agent is an epothilone;

a polymer comprising a hydrophilic portion and a hydrophobic portion; and

a liquid, wherein the polymer-agent conjugate and polymer comprising a hydrophilic portion and a hydrophobic portion are each independently suspended or dissolved in the liquid.

In some embodiments, the liquid is water. In some embodiments, the liquid is an organic solvent. In some embodiments, the organic solvent is miscible with water. In some embodiments, the organic solvent is acetone, ethanol, methanol, isopropyl alcohol, dichloromethane, acetonitrile, methyl ethyl ketone, tetrahydrofuran, butyl acetate, ethyl acetate, propyl acetate or dimethylformamide. In some embodiments, the liquid is a mixture of water and an organic solvent.

In some embodiments, the mixture further comprises a surfactant (e.g., PVA). In some embodiments, the mixture further comprises a compound comprising at least one acidic moiety.

In some embodiments, the hydrophobic polymer-agent conjugate and polymer comprising a hydrophilic portion and a hydrophobic portion are in the mixture as a particle (e.g., a particle described herein).

In some embodiments, the epothilone is selected from ixabepilone, epothilone B, epothilone D, BMS310705, dehydelone and ZK-EPO. In some embodiments, the epothilone is an epothilone described herein.

In another aspect, the invention features a mixture, the mixture comprising:

a first hydrophobic polymer;

a second polymer comprising a hydrophilic portion and a hydrophobic portion;

a first agent attached to the first or second polymer;

a second agent; and

a liquid, wherein the first polymer, the second polymer, the first agent, and the second agent are each independently suspended or dissolved in the liquid,

wherein at least one of the first or second agent is an epothilone.

In some embodiments, the epothilone is selected from ixabepilone, epothilone B, epothilone D, BMS310705, dehydelone and ZK-EPO. In some embodiments, the epothilone is an epothilone described herein. In some embodiments, at least one of the first or second agent is an epothilone, and the other of the first or second agent is an anti-cancer agent, e.g., an anti-cancer agent described herein. In some embodiments, the anti-cancer agent is an agent other than an epothilone.

In some embodiments, the first hydrophilic polymer, second polymer comprising a hydrophilic portion and a hydrophobic portion, first agent attached to the first or second polymer, and second agent are in the mixture as a particle (e.g., a particle described herein).

In some embodiments, the liquid is water. In some embodiments, the liquid is an organic solvent. In some embodiments, the organic solvent is acetone, ethanol, methanol, isopropyl alcohol, dichloromethane, acetonitrile, methyl ethyl ketone, tetrahydrofuran, butyl acetate, ethyl acetate, propyl acetate or dimethylformamide. In some embodiments, the liquid is a mixture of water and an organic solvent.

In yet another aspect, the invention features a composition (e.g., a pharmaceutical composition) comprising a plurality of particles described herein. In some embodiments, the composition further comprises an additional component. In some embodiments, the additional component is a pharmaceutically acceptable carrier. In some embodiments, the additional component is a surfactant or a polymer, e.g., a surfactant or a polymer not associated with a particle. In some embodiments, the surfactant is PEG, PVA, PVP, poloxamer, a polysorbate, a polyoxyethylene ester, a PEG-lipid (e.g., PEG-ceramide, d-alpha-tocopheryl polyethylene glycol 1000 succinate), 1,2-Distearoyl-sn-Glycero-3-[Phospho-rac-(1-glycerol)] or lecithin. In some embodiments, the surfactant is PVA and the PVA is from about 3 kDa to about 50 kDa (e.g., from about 5 kDa to about 45 kDa, about 7 kDa to about 42 kDa, from about 9 kDa to about 30 kDa, or from about 11 to about 28 kDa) and up to about 98% hydrolyzed (e.g., about 75-95%, about 80-90% hydrolyzed, or about 85% hydrolyzed). In some embodiments, the surfactant is polysorbate 80. In some embodiments, the surfactant is Solutol® HS 15. In some embodiments, the surfactant is present in an amount of up to about 35% by weight of the particle (e.g., up to about 20% by weight or up to about 25% by weight, from about 15% to about 35% by weight, from about 20% to about 30% by weight, or from about 23% to about 26% by weight).

In some embodiments, the composition further comprises a stabilizer or lyoprotectant, e.g., a stabilizer or lyoprotectant described herein. In some embodiments, the stabilizer or lyoprotectant is a carbohydrate (e.g., a carbohydrate described herein, such as, e.g., sucrose, cyclodextrin or a derivative of cyclodextrin (e.g. 2-hydroxypropyl-β-cyclodextrin)), salt, PEG, PVP or crown ether.

In some embodiments, the composition further comprises a solvent or suspending liquid (e.g., dextrose). In some embodiments, the composition further comprises one or more of the following: antioxidant, antibacterial, buffer, bulking agent, chelating agent, inert gas, tonicity agent or viscosity agent.

In yet another aspect, the invention features, a composition, e.g., a pharmaceutical composition, that comprises at least two structurally distinct types of particles described herein. The first and second type of particle can differ, e.g., by: the agent, the first polymer, the second polymer, or an additional component, e.g., a surfactant.

E.g., the composition can comprise a first particle comprising a first polymer-agent conjugate, and a second, structurally distinct polymer-agent conjugate. In an embodiment the first polymer-agent conjugate comprises a first agent, and the second polymer-agent conjugate comprises a second agent, wherein at least one of the first or second agent is an epothilone. In some embodiments, the epothilone is selected from ixabepilone, epothilone B, epothilone D, BMS310705, dehydelone and ZK-EPO. In some embodiments, the epothilone is an epothilone described herein. In some embodiments, at least one of the first or second agent is an epothilone, and the other of the first or second agent is an anti-cancer agent, e.g., an anti-cancer agent described herein. In some embodiments, the anti-cancer agent is an agent other than an epothilone.

In an embodiment the first or second polymer of the first type of particle and the corresponding polymer of the second type of particle can differ. E.g., they can differ by molecular weight, subunit composition (e.g., the first and second polymers are PLGA polymers having different ratios of ratio of lactic acid monomers to glycolic acid monomers), or subunit identity, e.g. a chitosan polymer and a PLGA polymer.

In an embodiment the first type of particle provides for a different profile for release of its agent as compared with the second type of particle, e.g., agent is released from the first type of particle with a first release profile and agent is released from the second type of particle with a second (different) release profile (the agent can be the same or different, e.g., two different anti-cancer agents). E.g., a bond between the agent and polymer in the first type of particle is more rapidly broken than a bond between the agent and polymer in the second type of particle. Thus, the release profile of one or more agents can be optimized.

In yet another aspect, the invention features a kit comprising a polymer-agent conjugate, particle or composition described herein and a device for delivery of the polymer-agent conjugate, particle or composition to a subject. In some embodiments, the device for delivery is an IV admixture bag, an IV infusion set, or a piggy back set.

In another aspect, the invention features a kit comprising a polymer-agent conjugate, particle or composition described herein and a container. In some embodiments, the container is a vial. In some embodiments, the vial is a sealed vial (e.g., under inert atmosphere). In some embodiments, the vial is sealed with a flexible seal, e.g., a rubber or silicone closure (e.g., polybutadiene or polyisoprene). In some embodiments, the vial is a light blocking vial. In some embodiments, the vial is substantially free of moisture.

In another aspect, the invention features a kit comprising a polymer-agent conjugate, particle or composition described herein and instructions for reconstituting the polymer-agent conjugate, particle or composition into a pharmaceutically acceptable composition. In embodiments the kit comprises a liquid for reconstitution, e.g., in a single or multi dose formant.

In another aspect, the invention features a kit comprising a polymer-agent conjugate, particle or composition described herein and pharmaceutically acceptable carrier.

In some embodiments, the kit comprises a single dosage unit of a polymer-agent conjugate, particle or composition described herein.

In another aspect, the invention features a method of storing a polymer-agent conjugate, particle or composition described herein, the method comprising providing a polymer-agent conjugate, article or composition described herein in a container, and storing the container for at least about 24 hours. In some embodiments, the container is stored at ambient conditions. In some embodiments, the container is stored at a temperature of less than or equal to about 4° C. In some embodiments, the container is a light blocking container. In some embodiments, the container is maintained under inert atmosphere. In some embodiments, the container is substantially free of moisture. In some embodiments, the container is a vial. In some embodiments, the vial is a sealed vial (e.g., under inert atmosphere). In some embodiments, vial is sealed with a rubber or silicone closure (e.g., polybutadiene or polyisoprene). In some embodiments, the vial is a light blocking vial. In some embodiments, the vial is substantially free of moisture.

In some embodiments, the invention features a dosage form comprising a polymer-agent conjugate, particle or composition described herein. In some embodiments, the dosage form is an oral dosage form. In some embodiments, the dosage form is a parenteral dosage form.

In some embodiments, the dosage form further comprises one or more of the following: antioxidant, antibacterial, buffer, bulking agent, chelating agent, inert gas, tonicity agent or viscosity agent.

In some embodiments, the dosage form is a parenteral dosage form (e.g., an intravenous dosage form). In some embodiments, the dosage form is an oral dosage form. In some embodiments, the dosage form is an inhaled dosage form. In some embodiments, the inhaled dosage form is delivered via nebulization, propellant or a dry powder device). In some embodiments, the dosage form is a topical dosage form. In some embodiments, the dosage form is a mucosal dosage form (e.g., a rectal dosage form or a vaginal dosage form). In some embodiments, the dosage form is an ophthalmic dosage form.

In some embodiments, the dosage form is a solid dosage form. In some embodiments, the dosage form is a liquid dosage form.

In yet another aspect, the invention features a single dosage unit comprising a polymer-agent conjugate, particle or composition described herein. In some embodiments, the single dosage unit is an intravenous dosage unit.

In another aspect, the invention features a method of preparing a liquid dosage form, the method comprising:

providing a polymer-agent conjugate, particle or composition described herein; and

dissolving or suspending the polymer-agent conjugate, particle or composition in a pharmaceutically acceptable carrier.

In one aspect, the invention features a method of instructing a user to prepare a liquid dosage form, the method comprising:

providing a polymer-agent conjugate, particle or composition described herein; and

instructing a user to dissolve or suspend the polymer-agent conjugate, particle or composition in a pharmaceutically acceptable carrier.

In one aspect, the invention features a method of evaluating a polymer-agent conjugate, particle or composition described herein, the method comprising:

subjecting a polymer-agent conjugate, particle or composition described herein to an analytical measurement and evaluating the particle or composition based on that measurement.

In some embodiments, the analytical measurement is evaluation of the presence or amount of an impurity or residual solvent. In some embodiments, the analytical measurement is a measurement of the polymer polydispersity index. In some embodiments, the analytical measurement is a measurement of the average particle size. In some embodiments, the analytical measurement is a measurement of the median particle size (Dv50). In some embodiments, the analytical measurement is a measurement of the particle size below which 90% of the volume of particles exists (Dv90). In some embodiments, the analytical measurement is a measurement of the particle polydispersity index.

In another aspect, the invention features a method of treating a disorder or disease described herein, the method comprising administering to a subject a polymer-agent conjugate, particle or composition described herein.

In an embodiment, the method further comprises administering agent not disposed in a particle, e.g., a particle described herein and/or not conjugated to a polymer, referred to herein as a “free” agent. In an embodiment, the agent disposed in a particle and the free agent are both anti-cancer agents, e.g., epothilones.

In an embodiment, the agent disposed in a particle and the free agent are the same anti-cancer agent. E.g., the agent is an epothilone selected from ixabepilone, epothilone B, epothilone D, BMS310705, dehydelone and ZK-EPO, or an epothilone described herein.

In an embodiment, the agent disposed in a particle and the free agent are different anti-cancer agents. E.g., one agent is an epothilone selected from ixabepilone, epothilone B, epothilone D, BMS310705, dehydelone and ZK-EPO, or an epothilone described herein and the other is an anti-cancer agent.

In one embodiment, the cancer is a cancer described herein. In one embodiment, the polymer-agent conjugate, particle or composition is administered in combination with one or more additional chemotherapeutic agent, e.g., a chemotherapeutic agent or combination of chemotherapeutic agents described herein.

In an embodiment, the polymer-agent conjugate comprises an agent coupled, e.g., via linkers, to a polymer described herein. In an embodiment, the polymer-agent conjugate comprises an agent, coupled via a linker shown in FIG. 1 to a polymer described herein.

In yet another aspect, the invention features a method of treating a proliferative disorder, e.g., a cancer, in a subject, e.g., a human. The method comprises: administering a polymer-agent conjugate, particle or composition, e.g., a polymer-agent conjugate, particle or composition described herein, to a subject in an amount effective to treat the disorder, to thereby treat the proliferative disorder.

In an embodiment, the polymer-agent conjugate comprises an anti-cancer agent such as an epothilone, coupled, e.g., via a linker, to a polymer described herein. In an embodiment, the polymer-agent conjugate comprises an anticancer agent such as an epothilone, coupled via a linker shown in FIG. 1 to a polymer, e.g., a polymer described herein. In an embodiment, the polymer-agent conjugate is a polymer-epothilone conjugate shown in FIG. 1.

In one embodiment, the polymer-agent conjugate, particle or composition is administered in combination with one or more additional chemotherapeutic agent, e.g., a chemotherapeutic agent or combination of chemotherapeutic agents described herein. For example, the polymer-agent conjugate, particle or composition can be administered in combination with an anti-metabolite such as capecitabine.

In one embodiment, the cancer is a cancer described herein. For example, the cancer can be a cancer of the bladder (including accelerated, locally advanced and metastatic bladder cancer), breast (e.g., estrogen receptor positive breast cancer; estrogen receptor negative breast cancer; HER-2 positive breast cancer; HER-2 negative breast cancer; progesterone receptor positive breast cancer; progesterone receptor negative breast cancer; estrogen receptor negative, HER-2 negative and progesterone receptor negative breast cancer (i.e., triple negative breast cancer); inflammatory breast cancer, colon (including colorectal cancer), kidney (e.g., transitional cell carcinoma), liver, lung (including small and non-small cell lung cancer (including lung adenocarcinoma, bronchioalveolar cancer and squamous cell cancer)), genitourinary tract, e.g., ovary (including fallopian tube and peritoneal cancers), cervix, prostate, testes, kidney, and ureter, lymphatic system, rectum, larynx, pancreas (including exocrine pancreatic carcinoma), esophagus, stomach, gall bladder, thyroid, skin (including squamous cell carcinoma), brain (including glioblastoma multiforme), head and neck (e.g., occult primary), and soft tissue (e.g., Kaposi's sarcoma (e.g., AIDS related Kaposi's sarcoma), leiomyosarcoma, angiosarcoma, and histiocytoma). Preferred cancers include breast cancer (e.g., metastatic or locally advanced breast cancer), prostate cancer (e.g., hormone refractory prostate cancer), renal cell carcinoma, lung cancer (e.g., non-small cell lung cancer and small cell lung cancer (including lung adenocarcinoma, bronchioalveolar cancer and squamous cell cancer) e.g., unresectable, locally advanced or metastatic non-small cell lung cancer and small cell lung cancer), pancreatic cancer, gastric cancer (e.g., metastatic gastric adenocarcinoma), colorectal cancer, rectal cancer, squamous cell cancer of the head and neck, lymphoma (Hodgkin's lymphoma or non-Hodgkin's lymphoma), renal cell carcinoma, carcinoma of the urothelium, soft tissue sarcoma (e.g., Kaposi's sarcoma (e.g., AIDS related Kaposi's sarcoma), leiomyosarcoma, angiosarcoma, and histiocytoma), gliomas, myeloma (e.g., multiple myeloma), melanoma (e.g., advanced or metastatic melanoma), germ cell tumors, ovarian cancer (e.g., advanced ovarian cancer, e.g., advanced fallopian tube or peritoneal cancer), and gastrointestinal cancer.

In one embodiment, the polymer-agent conjugate, particle or composition is administered by intravenous administration, e.g., an intravenous administration that is completed in a period equal to or less than 2 hours, 1.5 hours, 1 hour, 45 minutes or 30 minutes. In one embodiment, the polymer-agent conjugate, particle or composition is administered as a bolus infusion or intravenous push, e.g., over a period of 15 minutes, 10 minutes, 5 minutes or less.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein, and e.g., the polymer-agent conjugate, particle or composition is administered to the subject in an amount of the polymer-agent conjugate, particle or composition that includes 40 mg/m² or greater (e.g., 45 mg/m², 48 mg/m², 50 mg/m², 60 mg/m², 70 mg/m², 80 mg/m², 85 mg/m², 90 mg/m², 95 mg/m², 100 mg/m²), of an epothilone, e.g., ixabepilone, to thereby treat the disorder. In one embodiment, the polymer-agent conjugate, particle or composition is administered by intravenous administration over a period of about 30 minutes, 45 minutes, 60 minutes, 90 minutes, 120 minutes, 150 minutes or 180 minutes. In one embodiment, the subject is administered at least one additional dose of the polymer-agent conjugate, particle or composition, e.g., the subject is administered at least two, three, four, five, six, seven or eight additional doses of the polymer-agent conjugate, particle or composition. In one embodiment, the polymer-agent conjugate, particle or composition is administered once every one, two, three, four, five or six weeks. In one embodiment, the dosing schedule is not changed between doses. For example, when the dosing schedule is once every three weeks, an additional dose (or doses) is administered in three weeks. In one embodiment, when at least one additional dose is administered, the additional dose (or additional doses) is administered in an amount of the polymer-agent conjugate, particle or composition such that the polymer-agent conjugate, particle or composition includes 40 mg/m² or greater (e.g., 45 mg/m², 48 mg/m², 50 mg/m², 60 mg/m², 70 mg/m², 80 mg/m², 85 mg/m², 90 mg/m², 95 mg/m², 100 mg/m²) of an epothilone, e.g., ixabepilone. In one embodiment, when at least one additional dose is administered, the additional dose (or additional doses) is administered by intravenous administration over a period equal to or less than about 30 minutes, 45 minutes, 60 minutes, 90 minutes, 120 minutes, 150 minutes or 180 minutes.

In one embodiment, the polymer-agent conjugate, particle or composition includes a polymer-ixabepilone conjugate, e.g., a polymer-ixabepilone conjugate described herein, e.g., a polymer-ixabepilone conjugate comprising an ixabepilone molecule, coupled, e.g., via a linker, to a polymer described herein, and the polymer-ixabepilone conjugate, particle or composition is administered to the subject in an amount of the polymer-ixabepilone conjugate, particle or composition that includes 40 mg/m² or greater (e.g., 45 mg/m², 48 mg/m², 50 mg/m², 60 mg/m², 70 mg/m², 80 mg/m², 85 mg/m², 90 mg/m², 95 mg/m², 100 mg/m²), of ixabepilone, administered by intravenous administration over a period equal to or less than about 30 minutes, 45 minutes, 60 minutes, 90 minutes, 120 minutes, 150 minutes or 180 minutes, for at least two, three, fours, five or six doses, wherein the subject is administered a dose of the polymer-ixabepilone conjugate, particle or composition once every one, two, three, four, five or six weeks.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein, and at least two, three, four, five, six, seven or eight doses are administered to the subject and each dose is an amount of the polymer-ixabepilone conjugate, particle or composition that includes 40 mg/m² or greater (e.g., 45 mg/m², 48 mg/m², 50 mg/m², 60 mg/m², 70 mg/m², 80 mg/m², 85 mg/m², 90 mg/m², 95 mg/m², 100 mg/m²) of ixabepilone, to thereby treat the disorder. In one embodiment, the dose is administered once every one, two, three, four, five, six, seven or eight weeks. In one embodiment, a dose is administered once every three weeks. In one embodiment, each dose is administered by intravenous administration over a period of about 30 minutes, 45 minutes, 60 minutes, 90 minutes, 120 minutes, 150 minutes or 180 minutes. In one embodiment, the dosing schedule is not changed between doses. For example, when the dosing schedule is once every three weeks, an additional dose (or doses) is administered in three weeks.

In one embodiment, the polymer-agent conjugate, particle or composition includes a polymer-epothilone B conjugate, particle or composition, e.g., a polymer-epothilone B conjugate, particle or composition described herein and, e.g., a polymer-epothilone B conjugate comprising an epothilone B molecule, coupled, e.g., via a linker, to a polymer described herein, e.g., the polymer-epothilone B conjugate, particle or composition is administered in an amount of the polymer-epothilone B conjugate, particle or composition that includes 2.5 to 30 mg/m² (e.g., 2.5 mg/m², 5 mg/m², 6.5 mg/m², 8 mg/m², 10 mg/m², 12 mg/m², 15 mg/m², 18 mg/m², 20 mg/m², mg/m²) of epothilone B, to thereby treat the disorder. In one embodiment, the polymer-epothilone B conjugate, particle or composition is administered by intravenous administration over a period equal to or less than about 30 minutes, 45 minutes, 60 minutes, 90 minutes, 120 minutes, 150 minutes or 180 minutes. In one embodiment, the subject is administered at least one additional dose of the polymer-epothilone B conjugate, particle or composition, e.g., the subject is administered at least two, three, four, five, six, seven or eight additional doses of the polymer-epothilone B conjugate, particle or composition. In one embodiment, the polymer-epothilone B conjugate, particle or composition is administered once every one, two, three, four, five or six weeks. In one embodiment, the dosing schedule is not changed between doses. For example, when the dosing schedule is once every three weeks, an additional dose (or doses) is administered in three weeks. In one embodiment, when at least one additional dose is administered, the additional dose (or additional doses) is administered in an amount of the polymer-epothilone B conjugate, particle or composition that includes 2.5 to 30 mg/m² (e.g., 2.5 mg/m², 5 mg/m², 6.5 mg/m², 8 mg/m², 10 mg/m², 12 mg/m², 15 mg/m², 18 mg/m², 20 mg/m², 25 mg/m²) of the epothilone, e.g. epothilone B. In one embodiment, when at least one additional dose is administered, the additional dose (or additional doses) is administered by intravenous administration over a period equal to or less than about 30 minutes, 45 minutes, 60 minutes, 90 minutes, 120 minutes, 150 minutes or 180 minutes.

In one embodiment, the polymer-agent conjugate, particle or composition includes a polymer-epothilone B conjugate, particle or composition, e.g., a polymer-epothilone B conjugate, particle or composition described herein, e.g., a polymer-epothilone B conjugate comprising an epothilone B molecule, coupled, e.g., via a linker, to a polymer described herein, and the polymer-epothilone B conjugate, particle or composition is administered to the subject in an amount of the polymer-epothilone B conjugate, particle or composition that includes 2.5 to 30 mg/m² (e.g., 2.5 mg/m², 5 mg/m², 6.5 mg/m², 8 mg/m², 10 mg/m², 12 mg/m², 15 mg/m², 18 mg/m², mg/m², 25 mg/m²) of epothilone B, administered by intravenous administration over a period equal to or less than about 30 minutes, 45 minutes, 60 minutes, 90 minutes, 120 minutes, 150 minutes or 180 minutes, for at least two, three, fours, five or six doses, wherein the subject is administered a dose of the polymer-epothilone B conjugate, particle or composition once every one, two, three, four, five or six weeks.

In one embodiment, the polymer-agent conjugate, particle or composition includes a polymer-epothilone B conjugate, particle or composition, e.g., a polymer-epothilone B conjugate, particle or composition described herein, a e.g., a polymer-epothilone B conjugate comprising an epothilone B molecule, coupled, e.g., via a linker, to a polymer described herein, and at least two, three, four, five, six, seven or eight doses are administered to the subject and each dose is an amount of the polymer-epothilone B conjugate, particle or composition that includes 2.5 to 30 mg/m² (e.g., 2.5 mg/m², 5 mg/m², 6.5 mg/m², 8 mg/m², 10 mg/m², 12 mg/m², 15 mg/m², 18 mg/m², mg/m², 25 mg/m²) of epothilone B, to thereby treat the disorder. In one embodiment, the dose is administered once every one, two, three, four, five, six, seven or eight weeks. In one embodiment, a dose is administered once every three weeks. In one embodiment, each dose is administered by intravenous administration over a period equal to or less than about 30 minutes, 45 minutes, 60 minutes, 90 minutes, 120 minutes, 150 minutes or 180 minutes. In one embodiment, the dosing schedule is not changed between doses. For example, when the dosing schedule is once every three weeks, an additional dose (or doses) is administered in three weeks.

In one embodiment, the polymer-agent conjugate, particle or composition includes a polymer-epothilone D conjugate, particle or composition, e.g., a polymer-epothilone D conjugate, particle or composition described herein, e.g., a polymer-epothilone D conjugate comprising an epothilone D molecule, coupled, e.g., via a linker, to a polymer described herein, and, e.g., the polymer-epothilone D conjugate, particle or composition is administered in an amount of the polymer-epothilone D conjugate, particle or composition that includes 9 to 280 mg/m² (e.g., 9 mg/m², 16 mg/m², 20 mg/m², 50 mg/m², 100 mg/m², 150 mg/m², 185 mg/m², 200 mg/m², 220 mg/m², 240 mg/m², 260 mg/m², 280 mg/m², 300 mg/m², 320 mg/m², 340 mg/m², 360 mg/m², 370 mg/m²) of the epothilone, e.g., epothilone D, to thereby treat the disorder. In one embodiment, the polymer-epothilone D conjugate, particle or composition is administered by intravenous administration over a period equal to or less than about 30 minutes, 45 minutes, 60 minutes, 90 minutes, 120 minutes, 150 minutes or 180 minutes. In one embodiment, the subject is administered at least one additional dose of the polymer-epothilone D conjugate, particle or composition, e.g., the subject is administered at least two, three, four, five, six, seven or eight additional doses of the polymer-epothilone D conjugate, particle or composition. In one embodiment, the polymer-epothilone D conjugate, particle or composition is administered once every one, two, three, four, five or six weeks. In one embodiment, the dosing schedule is not changed between doses. For example, when the dosing schedule is once every three weeks, an additional dose (or doses) is administered in three weeks. In one embodiment, when at least one additional dose is administered, an additional dose (or additional doses) is administered in an amount of the polymer-epothilone D conjugate, particle or composition that includes 9 to 280 mg/m² (e.g., 9 mg/m², 16 mg/m², 20 mg/m², 50 mg/m², 100 mg/m², 150 mg/m², 185 mg/m², 200 mg/m², 220 mg/m², 240 mg/m², 260 mg/m², 280 mg/m², 300 mg/m², 320 mg/m², 340 mg/m², 360 mg/m², 370 mg/m²) of epothilone D. In one embodiment, when at least one additional dose is administered, the additional dose (or additional doses) is administered by intravenous administration over a period equal to or less than about 30 minutes, 45 minutes, 60 minutes, 90 minutes, 120 minutes, 150 minutes or 180 minutes.

In one embodiment, the polymer-agent conjugate, particle or composition includes a polymer-epothilone D conjugate, e.g., a polymer-epothilone D conjugate described herein, e.g., a polymer-epothilone D conjugate comprising an epothilone D molecule, coupled, e.g., via a linker, to a polymer described herein, and the polymer-epothilone D conjugate, particle or composition is administered to the subject in an amount of the polymer-epothilone D conjugate, particle or composition that includes 9 to 280 mg/m² (e.g., 9 mg/m², 16 mg/m², 20 mg/m², 50 mg/m², 100 mg/m², 150 mg/m², 185 mg/m², 200 mg/m², 220 mg/m², 240 mg/m², 260 mg/m², 280 mg/m², 300 mg/m², 320 mg/m², 340 mg/m², 360 mg/m², 370 mg/m²) of epothilone D, administered by intravenous administration over a period equal to or less than about 30 minutes, 45 minutes, 60 minutes, 90 minutes, 120 minutes, 150 minutes or 180 minutes, for at least two, three, fours, five or six doses, wherein the subject is administered a dose of the polymer-epothilone D conjugate, particle or composition once every one, two, three, four, five or six weeks.

In one embodiment, the polymer-agent conjugate, particle or composition includes a polymer-epothilone D conjugate, particle or composition, e.g., a polymer-epothilone D conjugate, particle or composition described herein, e.g., a polymer-epothilone D conjugate comprising an epothilone D molecule, coupled, e.g., via a linker, to a polymer described herein, and at least two, three, four, five, six, seven or eight doses are administered to the subject and each dose is an amount of the polymer-epothilone D conjugate, particle or composition that includes 9 to 280 mg/m² (e.g., 9 mg/m², 16 mg/m², 20 mg/m², 50 mg/m², 100 mg/m², 150 mg/m², 185 mg/m², 200 mg/m², 220 mg/m², 240 mg/m², 260 mg/m², 280 mg/m², 300 mg/m², 320 mg/m², 340 mg/m², 360 mg/m², 370 mg/m²) of epothilone D, to thereby treat the disorder. In one embodiment, the dose is administered once every one, two, three, four, five, six, seven or eight weeks. In one embodiment, a dose is administered once every three weeks. In one embodiment, each dose is administered by intravenous administration over a period equal to or less than about 30 minutes, 45 minutes, 60 minutes, 90 minutes, 120 minutes, 150 minutes or 180 minutes. In one embodiment, the dosing schedule is not changed between doses. For example, when the dosing schedule is once every three weeks, an additional dose (or doses) is administered in three weeks.

In one embodiment, the polymer-agent conjugate, particle or composition includes a polymer-BMS310705 conjugate, particle or composition, e.g., a polymer-BMS310705 conjugate, particle or composition described herein, e.g., a polymer-BMS310705 conjugate comprising a BMS310705 molecule, coupled, e.g., via a linker, to a polymer described herein, and, e.g., the polymer-BMS310705 conjugate, particle or composition is administered in an amount of the polymer-BMS310705 conjugate, particle or composition that includes 0.5 to 110 mg/m² (e.g., 0.6 mg/m², 1 mg/m², 5 mg/m², 10 mg/m², 15 mg/m², 20 mg/m², 24 mg/m², 25 mg/m², 30 mg/m², 35 mg/m², 40 mg/m², 45 mg/m², 50 mg/m², 55 mg/m², 60 mg/m², 65 mg/m², 70 mg/m², 75 mg/m², 80 mg/m², 85 mg/m², 90 mg/m², 95 mg/m², 100 mg/m², 105 mg/m², 105 mg/m², 110 mg/m², 115 mg/m², 120 mg/m², 125 mg/m², 130 mg/m², 135 mg/m², 140 mg/m²) of the epothilone, e.g., BMS310705, to thereby treat the disorder. In one embodiment, the polymer-BMS310705 conjugate, particle or composition is administered by intravenous administration over a period equal to or less than about 30 minutes, 45 minutes, 60 minutes, 90 minutes, 120 minutes, 150 minutes or 180 minutes. In one embodiment, the subject is administered at least one additional dose of the polymer-BMS310705 conjugate, particle or composition, e.g., the subject is administered at least two, three, four, five, six, seven or eight additional doses of the polymer-BMS310705 conjugate, particle or composition. In one embodiment, the dosing schedule is not changed between doses. For example, when the dosing schedule is once every three weeks, an additional dose (or doses) is administered in three weeks. In one embodiment, the polymer-BMS310705 conjugate, particle or composition is administered once every one, two, three, four, five or six weeks. In one embodiment, when at least one additional dose is administered, the additional dose (or additional doses) is administered in an amount of the polymer-BMS310705 conjugate, particle or composition that includes 0.5 to 110 mg/m² (e.g., 0.6 mg/m², 1 mg/m², 5 mg/m², 10 mg/m², 15 mg/m², 20 mg/m², 24 mg/m², 25 mg/m², 30 mg/m², 35 mg/m², 40 mg/m², 45 mg/m², 50 mg/m², 55 mg/m², 60 mg/m², 65 mg/m², 70 mg/m², 75 mg/m², 80 mg/m², 85 mg/m², 90 mg/m², 95 mg/m², 100 mg/m², 105 mg/m², 105 mg/m², 110 mg/m², 115 mg/m², 120 mg/m², 125 mg/m², 130 mg/m², 135 mg/m², 140 mg/m²) of the epothilone, e.g., BMS310705. In one embodiment, when at least one additional dose is administered, the additional dose (or additional doses) is administered by intravenous administration over a period equal to or less than about 30 minutes, 45 minutes, 60 minutes, 90 minutes, 120 minutes, 150 minutes or 180 minutes.

In one embodiment, the polymer-agent conjugate, particle or composition includes a polymer-BMS310705 conjugate, particle or composition, e.g., a polymer-BMS310705 conjugate, particle or composition described herein, e.g., a polymer-BMS310705 conjugate comprising a BMS310705 molecule, coupled, e.g., via a linker, to a polymer described herein, and the polymer-BMS310705 conjugate, particle or composition is administered to the subject in an amount of the polymer-BMS310705 conjugate, particle or composition that includes 0.5 to 110 mg/m² (e.g., 0.6 mg/m², 1 mg/m², 5 mg/m², 10 mg/m², 15 mg/m², 20 mg/m², 24 mg/m², 25 mg/m², mg/m², 35 mg/m², 40 mg/m², 45 mg/m², 50 mg/m², 55 mg/m², 60 mg/m², 65 mg/m², 70 mg/m², 75 mg/m², 80 mg/m², 85 mg/m², 90 mg/m², 95 mg/m², 100 mg/m², 105 mg/m², 105 mg/m², 110 mg/m², 115 mg/m², 120 mg/m², 125 mg/m², 130 mg/m², 135 mg/m², 140 mg/m²) of BMS310705, administered by intravenous administration over a period equal to or less than about 30 minutes, 45 minutes, 60 minutes, 90 minutes, 120 minutes, 150 minutes or 180 minutes, for at least two, three, fours, five or six doses, wherein the subject is administered a dose of the polymer-BMS310705 conjugate, particle or composition once every one, two, three, four, five or six weeks.

In one embodiment, the polymer-agent conjugate, particle or composition includes a polymer-BMS310705 conjugate, particle or composition, e.g., a polymer-BMS310705 conjugate, particle or composition described herein, e.g., a polymer-BMS310705 conjugate comprising a BMS310705 molecule, coupled, e.g., via a linker, to a polymer described herein, and at least two, three, four, five, six, seven or eight doses are administered to the subject and each dose is an amount of the polymer-BMS310705 conjugate, particle or composition that includes 0.5 to 110 mg/m² (e.g., 0.6 mg/m², 1 mg/m², 5 mg/m², 10 mg/m², 15 mg/m², 20 mg/m², 24 mg/m², 25 mg/m², mg/m², 35 mg/m², 40 mg/m², 45 mg/m², 50 mg/m², 55 mg/m², 60 mg/m², 65 mg/m², 70 mg/m², 75 mg/m², 80 mg/m², 85 mg/m², 90 mg/m², 95 mg/m², 100 mg/m², 105 mg/m², 105 mg/m², 110 mg/m², 115 mg/m², 120 mg/m², 125 mg/m², 130 mg/m², 135 mg/m², 140 mg/m²) of BMS310705, to thereby treat the disorder. In one embodiment, the dose is administered once every one, two, three, four, five, six, seven or eight weeks. In one embodiment, a dose is administered once every three weeks. In one embodiment, each dose is administered by intravenous administration over a period equal to or less than about 30 minutes, 45 minutes, 60 minutes, 90 minutes, 120 minutes, 150 minutes or 180 minutes. In one embodiment, the dosing schedule is not changed between doses. For example, when the dosing schedule is once every three weeks, an additional dose (or doses) is administered in three weeks.

In one embodiment, the polymer-agent conjugate, particle or composition includes a polymer-dehydelone conjugate, particle or composition, e.g., a polymer-dehydelone conjugate, particle or composition described herein, e.g., a polymer-dehydelone conjugate comprising a dehydelone molecule, coupled, e.g., via a linker, to a polymer described herein, and, e.g., the polymer-dehydelone conjugate, particle or composition is administered in an amount of the polymer-dehydelone conjugate, particle or composition that includes 0.5 to 35 mg/m² (e.g., 0.8 mg/m², 1 mg/m², 5 mg/m², 10 mg/m², 15 mg/m², 20 mg/m², 25 mg/m², 30 mg/m², 35 mg/m², 40 mg/m², 45 mg/m², 50 mg/m²) of the epothilone, e.g., dehydelone, to thereby treat the disorder. In one embodiment, the polymer-dehydelone conjugate, particle or composition is administered by intravenous administration over a period equal to or less than about 30 minutes, 45 minutes, 60 minutes, 90 minutes, 120 minutes, 150 minutes or 180 minutes. In one embodiment, the subject is administered at least one additional dose of the polymer-dehydelone conjugate, particle or composition, e.g., the subject is administered at least two, three, four, five, six, seven or eight additional doses of the polymer-dehydelone conjugate, particle or composition. In one embodiment, the polymer-dehydelone conjugate, particle or composition is administered once every one, two, three, four, five or six weeks. In one embodiment, the dosing schedule is not changed between doses. For example, when the dosing schedule is once every three weeks, an additional dose (or doses) is administered in three weeks. In one embodiment, when at least one additional dose is administered, the additional dose (or additional doses) is administered in an amount of the polymer-dehydelone conjugate, particle or composition that includes 0.5 to 35 mg/m² (e.g., 0.8 mg/m², 1 mg/m², 5 mg/m², 10 mg/m², 15 mg/m², 20 mg/m², 25 mg/m², 30 mg/m², 35 mg/m², 40 mg/m², 45 mg/m², 50 mg/m²) of dehydelone. In one embodiment, when at least one additional dose is administered, the additional dose (or additional doses) is administered by intravenous administration over a period equal to or less than about 30 minutes, 45 minutes, 60 minutes, 90 minutes, 120 minutes, 150 minutes or 180 minutes.

In one embodiment, the polymer-agent conjugate, particle or composition includes a polymer-dehydelone conjugate, particle or composition, e.g., a polymer-dehydelone conjugate, particle or composition described herein, e.g., a polymer-dehydelone conjugate comprising a dehydelone molecule, coupled, e.g., via a linker, to a polymer described herein, and the polymer-dehydelone conjugate, particle or composition is administered to the subject in an amount of the polymer-dehydelone conjugate, particle or composition that includes 0.5 to 35 mg/m² (e.g., 0.8 mg/m², 1 mg/m², 5 mg/m², 10 mg/m², 15 mg/m², 20 mg/m², 25 mg/m², 30 mg/m², 35 mg/m², 40 mg/m², 45 mg/m², 50 mg/m²) of dehydelone, administered by intravenous administration over a period equal to or less than about 30 minutes, 45 minutes, 60 minutes, 90 minutes, 120 minutes, 150 minutes or 180 minutes, for at least two, three, fours, five or six doses, wherein the subject is administered a dose of the polymer-dehydelone conjugate, particle or composition once every one, two, three, four, five or six weeks.

In one embodiment, the polymer-agent conjugate, particle or composition includes a polymer-dehydelone conjugate, particle or composition, e.g., a polymer-dehydelone conjugate, particle or composition described herein, e.g., a polymer-dehydelone conjugate comprising a dehydelone molecule, coupled, e.g., via a linker, to a polymer described herein, and at least two, three, four, five, six, seven or eight doses are administered to the subject and each dose is an amount of the polymer-dehydelone conjugate, particle or composition that includes 0.5 to 35 mg/m² (e.g., 0.8 mg/m², 1 mg/m², 5 mg/m², 10 mg/m², 15 mg/m², 20 mg/m², 25 mg/m², 30 mg/m², 35 mg/m², 40 mg/m², 45 mg/m², 50 mg/m²) of dehydelone to thereby treat the disorder. In one embodiment, the dose is administered once every one, two, three, four, five, six, seven or eight weeks. In one embodiment, a dose is administered once every three weeks. In one embodiment, each dose is administered by intravenous administration over a period equal to or less than about 30 minutes, 45 minutes, 60 minutes, 90 minutes, 120 minutes, 150 minutes or 180 minutes. In one embodiment, the dosing schedule is not changed between doses. For example, when the dosing schedule is once every three weeks, an additional dose (or doses) is administered in three weeks.

In one embodiment, the polymer-agent conjugate, particle or composition includes a polymer-ZK-EPO conjugate, particle or composition, e.g., a polymer-ZK-EPO conjugate, particle or composition described herein, e.g., a polymer-ZK-EPO conjugate comprising a ZK-EPO molecule, coupled, e.g., via a linker, to a polymer described herein, and, e.g., the polymer-ZK-EPO conjugate, particle or composition is administered in an amount of the polymer-ZK-EPO conjugate, particle or composition that includes 1 to 40 mg/m² (e.g., 2 mg/m², 5 mg/m², 10 mg/m², 16 mg/m², 20 mg/m², mg/m², 30 mg/m², 35 mg/m²) of the epothilone, e.g., ZK-EPO, to thereby treat the disorder. In one embodiment, the polymer-ZK-EPO conjugate, particle or composition is administered by intravenous administration over a period equal to or less than about 30 minutes, 45 minutes, 60 minutes, 90 minutes, 120 minutes, 150 minutes or 180 minutes. In one embodiment, the subject is administered at least one additional dose of the polymer-ZK-EPO conjugate, particle or composition, e.g., the subject is administered at least two, three, four, five, six, seven or eight additional doses of the polymer-ZK-EPO conjugate, particle or composition. In one embodiment, the polymer-ZK-EPO conjugate, particle or composition is administered once every one, two, three, four, five or six weeks. In one embodiment, the dosing schedule is not changed between doses. For example, when the dosing schedule is once every three weeks, an additional dose (or doses) is administered in three weeks. In one embodiment, when at least one additional dose is administered, the additional dose (or additional doses) is administered in an amount of the polymer-ZK-EPO conjugate, particle or composition that includes 1 to 40 mg/m² (e.g., 2 mg/m², 5 mg/m², 10 mg/m², 16 mg/m², 20 mg/m², 25 mg/m², 30 mg/m², 35 mg/m²) of ZK-EPO. In one embodiment, when at least one additional dose is administered, the additional dose (or additional doses) is administered by intravenous administration over a period equal to or less than about 30 minutes, 45 minutes, 60 minutes, 90 minutes, 120 minutes, 150 minutes or 180 minutes.

In one embodiment, the polymer-agent conjugate, particle or composition includes a polymer-ZK-EPO conjugate, particle or composition, e.g., a polymer-ZK-EPO conjugate, particle or composition described herein, e.g., a polymer-ZK-EPO conjugate comprising a ZK-EPO molecule, coupled, e.g., via a linker, to a polymer described herein, and the polymer-ZK-EPO conjugate, particle or composition is administered to the subject in an amount of the polymer-ZK-EPO conjugate, particle or composition that includes 1 to 40 mg/m² (e.g., 2 mg/m², 5 mg/m², 10 mg/m², 16 mg/m², 20 mg/m², 25 mg/m², 30 mg/m², 35 mg/m²) ZK-EPO, administered by intravenous administration over a period equal to or less than about 30 minutes, 45 minutes, 60 minutes, 90 minutes, 120 minutes, 150 minutes or 180 minutes, for at least two, three, fours, five or six doses, wherein the subject is administered a dose of the polymer-ZK-EPO conjugate, particle or composition once every one, two, three, four, five or six weeks.

In one embodiment, the polymer-agent conjugate, particle or composition includes a polymer-ZK-EPO conjugate, particle or composition, e.g., a polymer-ZK-EPO conjugate, particle or composition described herein, e.g., a polymer-ZK-EPO conjugate comprising a ZK-EPO molecule, coupled, e.g., via a linker, to a polymer described herein, and at least two, three, four, five, six, seven or eight doses are administered to the subject and each dose is an amount of the polymer-ZK-EPO conjugate, particle or composition that includes 1 to 40 mg/m² (e.g., 2 mg/m², 5 mg/m², 10 mg/m², 16 mg/m², 20 mg/m², 25 mg/m², 30 mg/m², 35 mg/m²) of ZK-EPO, to thereby treat the disorder. In one embodiment, the dose is administered once every one, two, three, four, five, six, seven or eight weeks. In one embodiment, a dose is administered once every three weeks. In one embodiment, each dose is administered by intravenous administration over a period equal to or less than about 30 minutes, 45 minutes, 60 minutes, 90 minutes, 120 minutes, 150 minutes or 180 minutes. In one embodiment, the dosing schedule is not changed between doses. For example, when the dosing schedule is once every three weeks, an additional dose (or doses) is administered in three weeks.

In one embodiment, the polymer-agent conjugate, particle or composition, e.g., a polymer-agent conjugate, particle or composition comprising an anticancer agent such as an epothilone, coupled, e.g., via a linker, to a polymer described herein, is administered once every three weeks in combination with one or more additional chemotherapeutic agent that is also administered once every three weeks. In one embodiment, the polymer-agent conjugate, particle or composition is administered once every three weeks in combination with one or more of the following chemotherapeutic agents: an antimetabolite (e.g., floxuridine, pemetrexed 5FU); an anthracycline (e.g., daunorubicin, epirubicin, idarubicin, mitoxantrone, valrubicin); a vinca alkaloid (e.g., vinblastine, vincristine, vindesine and vinorelbine); a topoisomerase inhibitor (e.g., topotecan, irinotecan, etoposide, teniposide, lamellarin D, camptothecin (e.g., IT-101)); and a platinum-based agent (e.g., cisplatin, carboplatin, oxaliplatin).

In one embodiment, the polymer-agent conjugate, particle or composition, e.g., a polymer-agent conjugate, particle or composition comprising an anticancer agent such as an epothilone, coupled, e.g., via a linker, to a polymer described herein, is administered once every two weeks in combination with one or more additional chemotherapeutic agent that is administered orally. In one embodiment, the polymer-agent conjugate, particle or composition is administered once every two weeks in combination with one or more of the following chemotherapeutic agents: capecitabine, estramustine, erlotinib, rapamycin, SDZ-RAD, CP-547632; AZD2171, sunitinib, sorafenib and everolimus.

In another aspect, the disclosure features a method of treating a chemotherapeutic sensitive, a chemotherapeutic refractory, a chemotherapeutic resistant, and/or a relapsed cancer. The method comprises: administering a polymer-agent conjugate, particle or composition, e.g., a polymer-agent conjugate, particle or composition described herein, to a subject, e.g., a human, in an amount effective to treat the disorder, to thereby treat the proliferative cancer.

In an embodiment, the polymer-agent conjugate comprises an anticancer agent such as an epothilone, coupled, e.g., via a linker, to a polymer described herein. In an embodiment, the polymer-agent conjugate comprises an anticancer agent such as an epothilone, coupled via a linker shown in FIG. 1 to a polymer, e.g., a polymer described herein. In an embodiment, the polymer-agent conjugate is a polymer-epothilone conjugate shown in FIG. 1.

In one embodiment, the cancer is refractory to, resistant to and/or relapsed during or after, treatment with, one or more of: a taxane (e.g., paclitaxel, docetaxel), an anthracycline (e.g., daunorubicin, epirubicin, idarubicin, mitoxantrone, valrubicin), an antimetabolite (e.g., an antifolate, a purine analogue, a pyrimidine analogue (e.g., capecitabine)), a vinca alkaloid (e.g., vinblastine, vincristine, vindesine and vinorelbine), a topoisomerase inhibitor (e.g., topotecan, irinotecan, etoposide, teniposide, lamellarin D, camptothecin (e.g., IT-101)) and a platinum-based agent (e.g., cisplatin, carboplatin, oxaliplatin). In one embodiment, the cancer is resistant to more than one chemotherapeutic agent, e.g., the cancer is a multidrug resistant cancer. In one embodiment, the cancer is resistant to one or more of a taxane, a platinum based agent and a vinca alkaloid, e.g., a taxane, a platinum based agent and a vinca alkaloid described herein.

In one embodiment, the polymer-agent conjugate, particle or composition is administered in combination with a second chemotherapeutic agent, e.g., a chemotherapeutic agent described herein. For example, the polymer-agent conjugate, particle or composition can be administered in combination with an antimetabolite such as capecitabine.

In one embodiment, the cancer is a cancer described herein. For example, the cancer can be carcinoma, including that of the bladder (including accelerated and metastatic bladder cancer), breast (e.g., estrogen receptor positive breast cancer; estrogen receptor negative breast cancer; HER-2 positive breast cancer; HER-2 negative breast cancer; progesterone receptor positive breast cancer; progesterone receptor negative breast cancer; estrogen receptor negative, HER-2 negative and progesterone receptor negative breast cancer (i.e., triple negative breast cancer); inflammatory breast cancer), colon (including colorectal cancer), kidney, liver, lung (including small and non-small cell lung cancer, lung adenocarcinoma and squamous cell cancer), genitourinary tract, e.g., ovary (including fallopian tube and peritoneal cancers) cervical, prostate and testes, lymphatic system, rectum, larynx, pancreas (including exocrine pancreatic carcinoma), esophagus, stomach, gall bladder, cervix, thyroid, and skin (including squamous cell carcinoma), brain (including glioblastoma multiforme), and head and neck. Preferred cancers include breast cancer (e.g., metastatic or locally advanced breast cancer), prostate cancer (e.g., hormone refractory prostate cancer), renal cell carcinoma, lung cancer (e.g., non-small cell lung cancer, small cell lung cancer, lung adenocarcinoma and squamous cell cancer, e.g., advanced non-small cell lung cancer, small cell lung cancer, lung adenocarcinoma, and squamous cell cancer), pancreatic cancer, gastric cancer (e.g., metastatic gastric adenocarcinoma), colorectal cancer, rectal cancer, squamous cell cancer of the head and neck, lymphoma (Hodgkin's or non-Hodgkin's lymphoma), renal cell carcinoma, carcinoma of the urothelium, soft tissue sarcoma, gliomas, melanoma (e.g., advanced or metastatic melanoma), germ cell tumors, ovarian cancer (e.g., advanced ovarian cancer, e.g., advanced fallopian tube or peritoneal cancer), glioblastoma and gastrointestinal cancer.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the polymer-ixabepilone conjugate, particle or composition is administered at a dose and/or dosing schedule described herein.

In one embodiment, the polymer-agent conjugate, particle or composition includes a polymer-epothilone B conjugate, particle or composition, e.g., a polymer-epothilone B conjugate, particle or composition described herein, e.g., a polymer-epothilone B conjugate comprising an epothilone B molecule, coupled, e.g., via a linker, to a polymer described herein. In an embodiment, the polymer-agent conjugate comprises an epothilone B molecule, coupled via a linker shown in FIG. 1 to a polymer, e.g., a polymer described herein. In an embodiment, the polymer-agent conjugate is a polymer-epothilone B conjugate shown in FIG. 1. In one embodiment, the polymer-epothilone B conjugate, particle or composition is administered at a dose and/or dosing schedule described herein.

In one embodiment, the polymer-agent conjugate, particle or composition includes a polymer-epothilone D conjugate, particle or composition, e.g., a polymer-epothilone D conjugate, particle or composition described herein, e.g., a polymer-epothilone D conjugate comprising an epothilone D molecule, coupled, e.g., via a linker, to a polymer described herein. In an embodiment, the polymer-agent conjugate comprises an epothilone D molecule, coupled via a linker shown in FIG. 1 to a polymer, e.g., a polymer described herein. In an embodiment, the polymer-agent conjugate is a polymer-epothilone D conjugate shown in FIG. 1. In one embodiment, the polymer-epothilone D conjugate, particle or composition is administered at a dose and/or dosing schedule described herein.

In one embodiment, the polymer-agent conjugate, particle or composition includes a polymer-BMS310705 conjugate, particle or composition, e.g., a polymer-BMS310705 conjugate, particle or composition described herein, e.g., a polymer-BMS310705 conjugate comprising a BMS310705 molecule, coupled, e.g., via a linker, to a polymer described herein. In an embodiment, the polymer-agent conjugate comprises a BMS310705 molecule, coupled via a linker shown in FIG. 1 to a polymer, e.g., a polymer described herein. In an embodiment, the polymer-agent conjugate is a polymer-BMS310705 conjugate shown in FIG. 1. In one embodiment, the polymer-BMS310705 conjugate, particle or composition is administered at a dose and/or dosing schedule described herein.

In one embodiment, the polymer-agent conjugate, particle or composition includes a polymer-dehydelone conjugate, particle or composition, e.g., a polymer-dehydelone conjugate, particle or composition described herein, e.g., a polymer-dehydelone conjugate comprising a dehydelone molecule, coupled, e.g., via a linker, to a polymer described herein. In an embodiment, the polymer-agent conjugate comprises a dehydelone molecule, coupled via a linker shown in FIG. 1 to a polymer, e.g., a polymer described herein. In an embodiment, the polymer-agent conjugate is a polymer-dehydelone conjugate shown in FIG. 1. In one embodiment, the polymer-dehydelone conjugate, particle or composition is administered at a dose and/or dosing schedule described herein.

In one embodiment, the polymer-agent conjugate, particle or composition includes a polymer-ZK-EPO conjugate, particle or composition, e.g., a polymer-ZK-EPO conjugate, particle or composition described herein, e.g., a polymer-ZK-EPO conjugate comprising a ZK-EPO molecule, coupled, e.g., via a linker, to a polymer described herein. In an embodiment, the polymer-agent conjugate comprises a ZK-EPO molecule, coupled via a linker shown in FIG. 1 to a polymer, e.g., a polymer described herein. In an embodiment, the polymer-agent conjugate is a polymer-ZK-EPO conjugate shown in FIG. 1. In one embodiment, the polymer-ZK-EPO conjugate, particle or composition is administered at a dose and/or dosing schedule described herein.

In one aspect, the disclosure features a method of treating metastatic or locally advanced breast cancer in a subject, e.g., a human. The method comprises: administering a polymer-agent conjugate, particle or composition, e.g., a polymer-agent conjugate, particle or composition described herein, to a subject in an amount effective to treat the cancer, to thereby treat the cancer.

In an embodiment, the polymer-agent conjugate comprises an anticancer agent such as an epothilone, coupled, e.g., via a linker, to a polymer described herein. In an embodiment, the polymer-agent conjugate comprises an anticancer agent such as an epothilone, coupled via a linker shown in FIG. 1 to a polymer, e.g., a polymer described herein. In an embodiment, the polymer-agent conjugate is a polymer-epothilone conjugate shown in FIG. 1.

In one embodiment, the breast cancer is estrogen receptor positive breast cancer; estrogen receptor negative breast cancer; HER-2 positive breast cancer; HER-2 negative breast cancer; progesterone receptor positive breast cancer; progesterone receptor negative breast cancer; estrogen receptor negative, HER-2 negative and progesterone receptor negative breast cancer (i.e., triple negative breast cancer) or inflammatory breast cancer.

In one embodiment, the polymer-agent conjugate, particle or composition is not administered in combination with a taxane.

In one embodiment, the polymer-agent conjugate, particle or composition is administered in combination with a HER-2 pathway inhibitor, e.g., a HER-2 inhibitor or a HER-2 receptor inhibitor. For example, the polymer-agent conjugate, particle or composition is administered with trastuzumab.

In some embodiments, the polymer-agent conjugate, particle or composition is administered in combination with a second chemotherapeutic agent. For example, the polymer-agent conjugate, particle or composition is administered in combination with a vascular endothelial growth factor (VEGF) pathway inhibitor, e.g., a VEGF inhibitor (e.g., bevacizumab) or VEGF receptor inhibitor (e.g., CP-547632 and AZD2171). In one embodiment, the polymer-agent conjugate, particle or composition is administered in combination with bevacizumab.

In some embodiments, the polymer-agent conjugate, particle or composition is administered in combination with an anthracycline (e.g., daunorubicin, doxorubicin, epirubicin, valrubicin and idarubicin).

In some embodiments, the polymer-agent conjugate, particle or composition is administered in combination with an anti-metabolite, e.g., an antifolate (e.g., floxuridine, pemetrexed) or pyrimidine analogue (e.g., 5FU)).

In some embodiments, the polymer-agent conjugate, particle or composition is administered in combination with an anthracycline (e.g., daunorubicin, doxorubicin, epirubicin, valrubicin and idarubicin) and an anti-metabolite (e.g., floxuridine, pemetrexed, 5FU).

In some embodiments, the polymer-agent conjugate, particle or composition is administered in combination with a platinum-based agent (e.g., cisplatin, carboplatin, oxaliplatin).

In some embodiments, the polymer-agent conjugate, particle or composition is administered in combination with an mTOR inhibitor. Non-limiting examples of mTOR inhibitors include rapamycin, everolimus, AP23573, CCI-779 and SDZ-RAD.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the polymer-ixabepilone conjugate, particle or composition is administered at a dose and/or dosing schedule described herein.

In one embodiment, the polymer-agent conjugate, particle or composition includes a polymer-epothilone B conjugate, particle or composition, e.g., a polymer-epothilone B conjugate, particle or composition described herein, e.g., a polymer-epothilone B conjugate comprising an epothilone B molecule, coupled, e.g., via a linker, to a polymer described herein. In an embodiment, the polymer-agent conjugate comprises an epothilone B molecule, coupled via a linker shown in FIG. 1 to a polymer, e.g., a polymer described herein. In an embodiment, the polymer-agent conjugate is a polymer-epothilone B conjugate shown in FIG. 1. In one embodiment, the polymer-epothilone B conjugate, particle or composition is administered at a dose and/or dosing schedule described herein.

In one embodiment, the polymer-agent conjugate, particle or composition includes a polymer-epothilone D conjugate, particle or composition, e.g., a polymer-epothilone D conjugate, particle or composition described herein, e.g., a polymer-epothilone D conjugate comprising an epothilone D molecule, coupled, e.g., via a linker, to a polymer described herein. In an embodiment, the polymer-agent conjugate comprises an epothilone D molecule, coupled via a linker shown in FIG. 1 to a polymer, e.g., a polymer described herein. In an embodiment, the polymer-agent conjugate is a polymer-epothilone D conjugate shown in FIG. 1. In one embodiment, the polymer-epothilone D conjugate, particle or composition is administered at a dose and/or dosing schedule described herein.

In one embodiment, the polymer-agent conjugate, particle or composition includes a polymer-BMS310705 conjugate, particle or composition, e.g., a polymer-BMS310705 conjugate, particle or composition described herein, e.g., a polymer-BMS310705 conjugate comprising a BMS310705 molecule, coupled, e.g., via a linker, to a polymer described herein. In an embodiment, the polymer-agent conjugate comprises a BMS310705 molecule, coupled via a linker shown in FIG. 1 to a polymer, e.g., a polymer described herein. In an embodiment, the polymer-agent conjugate is a polymer-BMS310705 conjugate shown in FIG. 1. In one embodiment, the polymer-BMS310705 conjugate, particle or composition is administered at a dose and/or dosing schedule described herein.

In one embodiment, the polymer-agent conjugate, particle or composition includes a polymer-dehydelone conjugate, particle or composition, e.g., a polymer-dehydelone conjugate, particle or composition described herein, e.g., a polymer-dehydelone conjugate comprising a dehydelone molecule, coupled, e.g., via a linker, to a polymer described herein. In an embodiment, the polymer-agent conjugate comprises a dehydelone molecule, coupled via a linker shown in FIG. 1 to a polymer, e.g., a polymer described herein. In an embodiment, the polymer-agent conjugate is a polymer-dehydelone conjugate shown in FIG. 1. In one embodiment, the polymer-dehydelone conjugate, particle or composition is administered at a dose and/or dosing schedule described herein.

In one embodiment, the polymer-agent conjugate, particle or composition includes a polymer-ZK-EPO conjugate, particle or composition, e.g., a polymer-ZK-EPO conjugate, particle or composition described herein, e.g., a polymer-ZK-EPO conjugate comprising a ZK-EPO molecule, coupled, e.g., via a linker, to a polymer described herein. In an embodiment, the polymer-agent conjugate comprises a ZK-EPO molecule, coupled via a linker shown in FIG. 1 to a polymer, e.g., a polymer described herein. In an embodiment, the polymer-agent conjugate is a polymer-ZK-EPO conjugate shown in FIG. 1. In one embodiment, the polymer-ZK-EPO conjugate, particle or composition is administered at a dose and/or dosing schedule described herein.

In one aspect, the disclosure features a method of treating metastatic or locally advanced breast cancer, e.g. a breast cancer described herein, in a subject, e.g., a human. The method comprises:

providing a subject that has metastatic or locally advanced breast cancer and has been treated with a chemotherapeutic agent which did not effectively treat the cancer (e.g., the subject has a chemotherapeutic refractory, a chemotherapeutic resistant and/or a relapsed cancer) or which had an unacceptable side effect (e.g., the subject has a chemotherapeutic sensitive cancer); and

administering a polymer-agent conjugate, particle or composition, e.g., a polymer-agent conjugate, particle or composition described herein, to a subject in an amount effective to treat the cancer, to thereby treat the cancer.

In an embodiment, the polymer-agent conjugate comprises an anticancer agent such as an epothilone, coupled, e.g., via a linker, to a polymer described herein. In an embodiment, the polymer-agent conjugate comprises an anticancer agent such as an epothilone, coupled via a linker shown in FIG. 1 to a polymer, e.g., a polymer described herein. In an embodiment, the polymer-agent conjugate is a polymer-epothilone conjugate shown in FIG. 1.

In one embodiment, the cancer is refractory to, resistant to, and/or relapsed with treatment with one or more of: a taxane, an anthracycline, pyrimidine analog, a vinca alkaloid (e.g., vinblastine, vincristine, vindesine and vinorelbine) and a platinum-based agent (e.g., cisplatin, carboplatin, oxaliplatin).

In one embodiment, the cancer is a multidrug resistant cancer.

In one embodiment, the polymer-agent conjugate, particle or composition is administered in combination with a pyrimidine analogue, e.g., a pyrimidine analogue described herein (e.g., capecitabine).

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the polymer-ixabepilone conjugate, particle or composition is administered at a dose and/or dosing schedule described herein.

In one embodiment, the polymer-agent conjugate, particle or composition includes a polymer-epothilone B conjugate, particle or composition, e.g., a polymer-epothilone B conjugate, particle or composition described herein, e.g., a polymer-epothilone B conjugate comprising an epothilone B molecule, coupled, e.g., via a linker, to a polymer described herein. In an embodiment, the polymer-agent conjugate comprises an epothilone B molecule, coupled via a linker shown in FIG. 1 to a polymer, e.g., a polymer described herein. In an embodiment, the polymer-agent conjugate is a polymer-epothilone B conjugate shown in FIG. 1. In one embodiment, the polymer-epothilone B conjugate, particle or composition is administered at a dose and/or dosing schedule described herein.

In one embodiment, the polymer-agent conjugate, particle or composition includes a polymer-epothilone D conjugate, particle or composition, e.g., a polymer-epothilone D conjugate, particle or composition described herein, e.g., a polymer-epothilone D conjugate comprising an epothilone D molecule, coupled, e.g., via a linker, to a polymer described herein. In an embodiment, the polymer-agent conjugate comprises an epothilone D molecule, coupled via a linker shown in FIG. 1 to a polymer, e.g., a polymer described herein. In an embodiment, the polymer-agent conjugate is a polymer-epothilone D conjugate shown in FIG. 1. In one embodiment, the polymer-epothilone D conjugate, particle or composition is administered at a dose and/or dosing schedule described herein.

In one embodiment, the polymer-agent conjugate, particle or composition includes a polymer-BMS310705 conjugate, particle or composition, e.g., a polymer-BMS310705 conjugate, particle or composition described herein, e.g., a polymer-BMS310705 conjugate comprising a BMS310705 molecule, coupled, e.g., via a linker, to a polymer described herein. In an embodiment, the polymer-agent conjugate comprises a BMS310705 molecule, coupled via a linker shown in FIG. 1 to a polymer, e.g., a polymer described herein. In an embodiment, the polymer-agent conjugate is a polymer-BMS310705 conjugate shown in FIG. 1. In one embodiment, the polymer-BMS310705 conjugate, particle or composition is administered at a dose and/or dosing schedule described herein.

In one embodiment, the polymer-agent conjugate, particle or composition includes a polymer-dehydelone conjugate, particle or composition, e.g., a polymer-dehydelone conjugate, particle or composition described herein, e.g., a polymer-dehydelone conjugate comprising a dehydelone molecule, coupled, e.g., via a linker, to a polymer described herein. In an embodiment, the polymer-agent conjugate comprises a dehydelone molecule, coupled via a linker shown in FIG. 1 to a polymer, e.g., a polymer described herein. In an embodiment, the polymer-agent conjugate is a polymer-dehydelone conjugate shown in FIG. 1. In one embodiment, the polymer-dehydelone conjugate, particle or composition is administered at a dose and/or dosing schedule described herein.

In one embodiment, the polymer-agent conjugate, particle or composition includes a polymer-ZK-EPO conjugate, particle or composition, e.g., a polymer-ZK-EPO conjugate, particle or composition described herein, e.g., a polymer-ZK-EPO conjugate comprising a ZK-EPO molecule, coupled, e.g., via a linker, to a polymer described herein. In an embodiment, the polymer-agent conjugate comprises a ZK-EPO molecule, coupled via a linker shown in FIG. 1 to a polymer, e.g., a polymer described herein. In an embodiment, the polymer-agent conjugate is a polymer-ZK-EPO conjugate shown in FIG. 1. In one embodiment, the polymer-ZK-EPO conjugate, particle or composition is administered at a dose and/or dosing schedule described herein.

In one aspect, the disclosure features a method of treating hormone refractory prostate cancer in a subject, e.g., a human. The method comprises: administering a polymer-agent conjugate, particle or composition e.g., a polymer-agent conjugate, particle or composition described herein, to a subject in an amount effective to treat the cancer, to thereby treat the cancer.

In an embodiment, the polymer-agent conjugate comprises an anticancer agent such as an epothilone, coupled, e.g., via a linker, to a polymer described herein. In an embodiment, the polymer-agent conjugate comprises an anticancer agent such as an epothilone, coupled via a linker shown in FIG. 1 to a polymer, e.g., a polymer described herein. In an embodiment, the polymer-agent conjugate is a polymer-epothilone conjugate shown in FIG. 1.

In one embodiment, the polymer-agent conjugate, particle or composition is not administered in combination with a taxane.

In one embodiment, the polymer-agent conjugate, particle or composition is administered in combination with prednisone.

In one embodiment, the polymer-agent conjugate, particle or composition is administered in combination with estramustine.

In one embodiment, the polymer-agent conjugate, particle or composition is administered in combination with an anthracenedione (e.g., mitoxantrone) and prednisone.

In one embodiment, the polymer-agent conjugate, particle or composition is administered in combination with a vascular endothelial growth factor (VEGF) pathway inhibitor, e.g., a VEGF inhibitor (e.g., bevacizumab) or VEGF receptor inhibitor (e.g., CP-547632 and AZD2171).

In one embodiment, the polymer-agent conjugate, particle or composition is administered in combination with an mTOR inhibitor. Non-limiting examples of mTOR inhibitors include rapamycin, everolimus, AP23573, CCI-779, and SDZ-RAD.

In one embodiment, the polymer-agent conjugate, particle or composition is administered in combination with a platinum-based agent (e.g., cisplatin, carboplatin, oxaliplatin).

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the polymer-ixabepilone conjugate, particle or composition is administered at a dose and/or dosing schedule described herein.

In one embodiment, the polymer-agent conjugate, particle or composition includes a polymer-epothilone B conjugate, particle or composition, e.g., a polymer-epothilone B conjugate, particle or composition described herein, e.g., a polymer-epothilone B conjugate comprising an epothilone B molecule, coupled, e.g., via a linker, to a polymer described herein. In an embodiment, the polymer-agent conjugate comprises an epothilone B molecule, coupled via a linker shown in FIG. 1 to a polymer, e.g., a polymer described herein. In an embodiment, the polymer-agent conjugate is a polymer-epothilone B conjugate shown in FIG. 1. In one embodiment, the polymer-epothilone B conjugate, particle or composition is administered at a dose and/or dosing schedule described herein.

In one embodiment, the polymer-agent conjugate, particle or composition includes a polymer-epothilone D conjugate, particle or composition, e.g., a polymer-epothilone D conjugate, particle or composition described herein, e.g., a polymer-epothilone D conjugate comprising an epothilone D molecule, coupled, e.g., via a linker, to a polymer described herein. In an embodiment, the polymer-agent conjugate comprises an epothilone D molecule, coupled via a linker shown in FIG. 1 to a polymer, e.g., a polymer described herein. In an embodiment, the polymer-agent conjugate is a polymer-epothilone D conjugate shown in FIG. 1. In one embodiment, the polymer-epothilone D conjugate, particle or composition is administered at a dose and/or dosing schedule described herein.

In one embodiment, the polymer-agent conjugate, particle or composition includes a polymer-BMS310705 conjugate, particle or composition, e.g., a polymer-BMS310705 conjugate, particle or composition described herein, e.g., a polymer-BMS310705 conjugate comprising a BMS310705 molecule, coupled, e.g., via a linker, to a polymer described herein. In an embodiment, the polymer-agent conjugate comprises a BMS310705 molecule, coupled via a linker shown in FIG. 1 to a polymer, e.g., a polymer described herein. In an embodiment, the polymer-agent conjugate is a polymer-BMS310705 conjugate shown in FIG. 1. In one embodiment, the polymer-BMS310705 conjugate, particle or composition is administered at a dose and/or dosing schedule described herein.

In one embodiment, the polymer-agent conjugate, particle or composition includes a polymer-dehydelone conjugate, particle or composition, e.g., a polymer-dehydelone conjugate, particle or composition described herein, e.g., a polymer-dehydelone conjugate comprising a dehydelone molecule, coupled, e.g., via a linker, to a polymer described herein. In an embodiment, the polymer-agent conjugate comprises a dehydelone molecule, coupled via a linker shown in FIG. 1 to a polymer, e.g., a polymer described herein. In an embodiment, the polymer-agent conjugate is a polymer-dehydelone conjugate shown in FIG. 1. In one embodiment, the polymer-dehydelone conjugate, particle or composition is administered at a dose and/or dosing schedule described herein.

In one embodiment, the polymer-agent conjugate, particle or composition includes a polymer-ZK-EPO conjugate, particle or composition, e.g., a polymer-ZK-EPO conjugate, particle or composition described herein, e.g., a polymer-ZK-EPO conjugate comprising a ZK-EPO molecule, coupled, e.g., via a linker, to a polymer described herein. In an embodiment, the polymer-agent conjugate comprises a ZK-EPO molecule, coupled via a linker shown in FIG. 1 to a polymer, e.g., a polymer described herein. In an embodiment, the polymer-agent conjugate is a polymer-ZK-EPO conjugate shown in FIG. 1. In one embodiment, the polymer-ZK-EPO conjugate, particle or composition is administered at a dose and/or dosing schedule described herein.

In one aspect, the disclosure features a method of treating hormone refractory prostate cancer in a subject, e.g., a human. The method comprises:

providing a subject who has hormone refractory prostate cancer and has been treated with a chemotherapeutic agent that did not effectively treat the cancer (e.g., the subject has a chemotherapeutic refractory, chemotherapeutic resistant and/or relapsed cancer) or who had unacceptable side effect (e.g., the subject has a chemotherapeutic sensitive cancer); and

administering a polymer-agent conjugate, particle or composition e.g., a polymer-agent conjugate, particle or composition described herein, to a subject in an amount effective to treat the cancer, to thereby treat the cancer.

In an embodiment, the polymer-agent conjugate comprises an anticancer agent such as an epothilone, coupled, e.g., via a linker, to a polymer described herein. In an embodiment, the polymer-agent conjugate comprises an anticancer agent such as an epothilone, coupled via a linker shown in FIG. 1 to a polymer, e.g., a polymer described herein. In an embodiment, the polymer-agent conjugate is a polymer-epothilone conjugate shown in FIG. 1.

In one embodiment, the subject has been treated with a taxane which did not effectively treat the cancer (e.g., the subject has a taxane refractory, a taxane resistant and/or a relapsed cancer).

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the polymer-ixabepilone conjugate, particle or composition is administered at a dose and/or dosing schedule described herein.

In one embodiment, the polymer-agent conjugate, particle or composition includes a polymer-epothilone B conjugate, particle or composition, e.g., a polymer-epothilone B conjugate, particle or composition described herein, e.g., a polymer-epothilone B conjugate comprising an epothilone B molecule, coupled, e.g., via a linker, to a polymer described herein. In an embodiment, the polymer-agent conjugate comprises an epothilone B molecule, coupled via a linker shown in FIG. 1 to a polymer, e.g., a polymer described herein. In an embodiment, the polymer-agent conjugate is a polymer-epothilone B conjugate shown in FIG. 1. In one embodiment, the polymer-epothilone B conjugate, particle or composition is administered at a dose and/or dosing schedule described herein.

In one embodiment, the polymer-agent conjugate, particle or composition includes a polymer-epothilone D conjugate, particle or composition, e.g., a polymer-epothilone D conjugate, particle or composition described herein, e.g., a polymer-epothilone D conjugate comprising an epothilone D molecule, coupled, e.g., via a linker, to a polymer described herein. In an embodiment, the polymer-agent conjugate comprises an epothilone D molecule, coupled via a linker shown in FIG. 1 to a polymer, e.g., a polymer described herein. In an embodiment, the polymer-agent conjugate is a polymer-epothilone D conjugate shown in FIG. 1. In one embodiment, the polymer-epothilone D conjugate, particle or composition is administered at a dose and/or dosing schedule described herein.

In one embodiment, the polymer-agent conjugate, particle or composition includes a polymer-BMS310705 conjugate, particle or composition, e.g., a polymer-BMS310705 conjugate, particle or composition described herein, e.g., a polymer-BMS310705 conjugate comprising a BMS310705 molecule, coupled, e.g., via a linker, to a polymer described herein. In an embodiment, the polymer-agent conjugate comprises a BMS310705 molecule, coupled via a linker shown in FIG. 1 to a polymer, e.g., a polymer described herein. In an embodiment, the polymer-agent conjugate is a polymer-BMS310705 conjugate shown in FIG. 1. In one embodiment, the polymer-BMS310705 conjugate, particle or composition is administered at a dose and/or dosing schedule described herein.

In one embodiment, the polymer-agent conjugate, particle or composition includes a polymer-dehydelone conjugate, particle or composition, e.g., a polymer-dehydelone conjugate, particle or composition described herein, e.g., a polymer-dehydelone conjugate comprising a dehydelone molecule, coupled, e.g., via a linker, to a polymer described herein. In an embodiment, the polymer-agent conjugate comprises a dehydelone molecule, coupled via a linker shown in FIG. 1 to a polymer, e.g., a polymer described herein. In an embodiment, the polymer-agent conjugate is a polymer-dehydelone conjugate shown in FIG. 1. In one embodiment, the polymer-dehydelone conjugate, particle or composition is administered at a dose and/or dosing schedule described herein.

In one embodiment, the polymer-agent conjugate, particle or composition includes a polymer-ZK-EPO conjugate, particle or composition, e.g., a polymer-ZK-EPO conjugate, particle or composition described herein, e.g., a polymer-ZK-EPO conjugate comprising a ZK-EPO molecule, coupled, e.g., via a linker, to a polymer described herein. In an embodiment, the polymer-agent conjugate comprises a ZK-EPO molecule, coupled via a linker shown in FIG. 1 to a polymer, e.g., a polymer described herein. In an embodiment, the polymer-agent conjugate is a polymer-ZK-EPO conjugate shown in FIG. 1. In one embodiment, the polymer-ZK-EPO conjugate, particle or composition is administered at a dose and/or dosing schedule described herein.

In one aspect, the disclosure features a method of treating renal cell carcinoma in a subject, e.g., a human. The method comprises: administering a polymer-agent conjugate, particle or composition, e.g., a polymer-agent conjugate, particle or composition described herein, to a subject in an amount effective to treat the carcinoma, to thereby treat the carcinoma.

In an embodiment, the polymer-agent conjugate comprises an anticancer agent such as an epothilone, coupled, e.g., via a linker, to a polymer described herein. In an embodiment, the polymer-agent conjugate comprises an anticancer agent such as an epothilone, coupled via a linker shown in FIG. 1 to a polymer, e.g., a polymer described herein. In an embodiment, the polymer-agent conjugate is a polymer-epothilone conjugate shown in FIG. 1.

In one embodiment, the polymer-agent conjugate, particle or composition is administered in combination with an mTOR inhibitor. Exemplary mTOR inhibitors include rapamycin, everolimus, AP23573, CCI-779 and SDZ-RAD.

In one embodiment, the polymer-agent conjugate, particle or composition is administered in combination with a vascular endothelial growth factor (VEGF) pathway inhibitor, e.g., a VEGF inhibitor or VEGF receptor inhibitor. In one embodiment, the VEGF inhibitor is bevacizumab. In another embodiment, the VEGF receptor inhibitor is selected from CP-547632, AZD2171, sorafinib and sunitinib.

In one embodiment, the polymer-agent conjugate, particle or composition is administered in combination with interleukin-2.

In one embodiment, the polymer-agent conjugate, particle or composition is administered in combination with interferon.

In one embodiment, the polymer-agent conjugate, particle or composition is administered in combination with a pyrimidine analogue, e.g., capecitabine.

In one embodiment, the com polymer-agent conjugate, particle or composition position is administered in combination with an anti-metabolite, e.g., an antifolate, e.g., floxuridine or pyrimidine analogue, e.g., 5FU, and/or a nucleoside analog, e.g., gemcitabine.

In one embodiment, the polymer-agent conjugate, particle or composition is administered in combination with an anthracycline (e.g., daunorubicin, doxorubicin, epirubicin, valrubicin or idarubicin).

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the polymer-ixabepilone conjugate, particle or composition is administered at a dose and/or dosing schedule described herein.

In one embodiment, the polymer-agent conjugate, particle or composition includes a polymer-epothilone B conjugate, particle or composition, e.g., a polymer-epothilone B conjugate, particle or composition described herein, e.g., a polymer-epothilone B conjugate comprising an epothilone B molecule, coupled, e.g., via a linker, to a polymer described herein. In an embodiment, the polymer-agent conjugate comprises an epothilone B molecule, coupled via a linker shown in FIG. 1 to a polymer, e.g., a polymer described herein. In an embodiment, the polymer-agent conjugate is a polymer-epothilone B conjugate shown in FIG. 1. In one embodiment, the polymer-epothilone B conjugate, particle or composition is administered at a dose and/or dosing schedule described herein.

In one embodiment, the polymer-agent conjugate, particle or composition includes a polymer-epothilone D conjugate, particle or composition, e.g., a polymer-epothilone D conjugate, particle or composition described herein, e.g., a polymer-epothilone D conjugate comprising an epothilone D molecule, coupled, e.g., via a linker, to a polymer described herein. In an embodiment, the polymer-agent conjugate comprises an epothilone D molecule, coupled via a linker shown in FIG. 1 to a polymer, e.g., a polymer described herein. In an embodiment, the polymer-agent conjugate is a polymer-epothilone D conjugate shown in FIG. 1. In one embodiment, the polymer-epothilone D conjugate, particle or composition is administered at a dose and/or dosing schedule described herein.

In one embodiment, the polymer-agent conjugate, particle or composition includes a polymer-BMS310705 conjugate, particle or composition, e.g., a polymer-BMS310705 conjugate, particle or composition described herein, e.g., a polymer-BMS310705 conjugate comprising a BMS310705 molecule, coupled, e.g., via a linker, to a polymer described herein. In an embodiment, the polymer-agent conjugate comprises a BMS310705 molecule, coupled via a linker shown in FIG. 1 to a polymer, e.g., a polymer described herein. In an embodiment, the polymer-agent conjugate is a polymer-BMS310705 conjugate shown in FIG. 1. In one embodiment, the polymer-BMS310705 conjugate, particle or composition is administered at a dose and/or dosing schedule described herein.

In one embodiment, the polymer-agent conjugate, particle or composition includes a polymer-dehydelone conjugate, particle or composition, e.g., a polymer-dehydelone conjugate, particle or composition described herein, e.g., a polymer-dehydelone conjugate comprising a dehydelone molecule, coupled, e.g., via a linker, to a polymer described herein. In an embodiment, the polymer-agent conjugate comprises a dehydelone molecule, coupled via a linker shown in FIG. 1 to a polymer, e.g., a polymer described herein. In an embodiment, the polymer-agent conjugate is a polymer-dehydelone conjugate shown in FIG. 1. In one embodiment, the polymer-dehydelone conjugate, particle or composition is administered at a dose and/or dosing schedule described herein.

In one embodiment, the polymer-agent conjugate, particle or composition includes a polymer-ZK-EPO conjugate, particle or composition, e.g., a polymer-ZK-EPO conjugate, particle or composition described herein, e.g., a polymer-ZK-EPO conjugate comprising a ZK-EPO molecule, coupled, e.g., via a linker, to a polymer described herein. In an embodiment, the polymer-agent conjugate comprises a ZK-EPO molecule, coupled via a linker shown in FIG. 1 to a polymer, e.g., a polymer described herein. In an embodiment, the polymer-agent conjugate is a polymer-ZK-EPO conjugate shown in FIG. 1. In one embodiment, the polymer-ZK-EPO conjugate, particle or composition is administered at a dose and/or dosing schedule described herein.

In one aspect, the disclosure features a method of treating renal cell carcinoma in a subject, e.g., a human. The method comprises:

providing a subject who has renal cell carcinoma and has been treated with a chemotherapeutic agent that did not effectively treat the carcinoma (e.g., the subject has a chemotherapeutic refractory, a chemotherapeutic resistant and/or a relapsed carcinoma) or who had an unacceptable side effect (e.g., the subject has a chemotherapeutic sensitive carcinoma); and

administering a polymer-agent conjugate, particle or composition, e.g., a polymer-agent conjugate, particle or composition described herein, to a subject in an amount effective to treat the carcinoma, to thereby treat the carcinoma.

In an embodiment, the polymer-agent conjugate comprises an anticancer agent such as an epothilone, coupled, e.g., via a linker, to a polymer described herein.

In one embodiment, the subject has been treated with a taxane which did not effectively treat the carcinoma (e.g., the subject has a taxane refractory, a taxane resistant and/or a relapsed carcinoma).

In one embodiment, the subject has been treated with an mTOR inhibitor which did not effectively treat the carcinoma (e.g., the subject has been treated with rapamycin, everolimus, AP23573, CCI-779 or SDZ-RAD which did not effectively treat the carcinoma).

In one embodiment, the subject has been treated with a vascular endothelial growth factor (VEGF) pathway inhibitor (e.g., a VEGF inhibitor or a VEGF receptor inhibitor) which did not effectively treat the carcinoma (e.g., the subject has been treated with bevacizumab, CP-547632, AZD2171, sunitinib or sorafinib which did not effectively treat the carcinoma).

In one embodiment, the subject has been treated with interleukin-2 which did not effectively treat the carcinoma.

In one embodiment, the subject has been treated with a nucleoside analog which did not effectively treat the carcinoma (e.g., the subject has been treated with gemcitabine which did not effectively treat the carcinoma).

In one embodiment, the subject has been treated with an anti-metabolite which did not effectively treat the carcinoma (e.g., the subject has been treated with an antifolate, e.g., floxuridine, pemetrexed, or a pyrimidine analog, e.g., capecitabine or 5FU, which did not effectively treat the carcinoma).

In one embodiment, the subject has been treated with an anthracycline which did not effectively treat the carcinoma (e.g., the subject has been treated with daunorubicin, doxorubicin, epirubicin, valrubicin or idarubicin which did not effectively treat the carcinoma).

In one embodiment, the polymer-agent conjugate, particle or composition is administered in combination with an mTOR inhibitor, e.g., rapamycin, everolimus, AP23573, CCI-779 or SDZ-RAD. In one embodiment, the subject has been treated with a VEGF pathway inhibitor (e.g., a VEGF inhibitor or a VEGF receptor inhibitor) which did not effectively treat the cancer (e.g., the subject has been treated with bevacizumab, CP-547632, AZD2171, sunitinib or sorafinib which did not effectively treat the carcinoma), and the polymer-agent conjugate, particle or composition is administered to the subject in combination with an mTOR inhibitor, e.g., everolimus.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the polymer-ixabepilone conjugate, particle or composition is administered at a dose and/or dosing schedule described herein.

In one embodiment, the polymer-agent conjugate, particle or composition includes a polymer-epothilone B conjugate, particle or composition, e.g., a polymer-epothilone B conjugate, particle or composition described herein, e.g., a polymer-epothilone B conjugate comprising an epothilone B molecule, coupled, e.g., via a linker, to a polymer described herein. In an embodiment, the polymer-agent conjugate comprises an epothilone B molecule, coupled via a linker shown in FIG. 1 to a polymer, e.g., a polymer described herein. In an embodiment, the polymer-agent conjugate is a polymer-epothilone B conjugate shown in FIG. 1. In one embodiment, the polymer-epothilone B conjugate, particle or composition is administered at a dose and/or dosing schedule described herein.

In one embodiment, the polymer-agent conjugate, particle or composition includes a polymer-epothilone D conjugate, particle or composition, e.g., a polymer-epothilone D conjugate, particle or composition described herein, e.g., a polymer-epothilone D conjugate comprising an epothilone D molecule, coupled, e.g., via a linker, to a polymer described herein. In an embodiment, the polymer-agent conjugate comprises an epothilone D molecule, coupled via a linker shown in FIG. 1 to a polymer, e.g., a polymer described herein. In an embodiment, the polymer-agent conjugate is a polymer-epothilone D conjugate shown in FIG. 1. In one embodiment, the polymer-epothilone D conjugate, particle or composition is administered at a dose and/or dosing schedule described herein.

In one embodiment, the polymer-agent conjugate, particle or composition includes a polymer-BMS310705 conjugate, particle or composition, e.g., a polymer-BMS310705 conjugate, particle or composition described herein, e.g., a polymer-BMS310705 conjugate comprising a BMS310705 molecule, coupled, e.g., via a linker, to a polymer described herein. In an embodiment, the polymer-agent conjugate comprises a BMS310705 molecule, coupled via a linker shown in FIG. 1 to a polymer, e.g., a polymer described herein. In an embodiment, the polymer-agent conjugate is a polymer-BMS310705 conjugate shown in FIG. 1. In one embodiment, the polymer-BMS310705 conjugate, particle or composition is administered at a dose and/or dosing schedule described herein.

In one embodiment, the polymer-agent conjugate, particle or composition includes a polymer-dehydelone conjugate, particle or composition, e.g., a polymer-dehydelone conjugate, particle or composition described herein, e.g., a polymer-dehydelone conjugate comprising a dehydelone molecule, coupled, e.g., via a linker, to a polymer described herein. In an embodiment, the polymer-agent conjugate comprises a dehydelone molecule, coupled via a linker shown in FIG. 1 to a polymer, e.g., a polymer described herein. In an embodiment, the polymer-agent conjugate is a polymer-dehydelone conjugate shown in FIG. 1. In one embodiment, the polymer-dehydelone conjugate, particle or composition is administered at a dose and/or dosing schedule described herein.

In one embodiment, the polymer-agent conjugate, particle or composition includes a polymer-ZK-EPO conjugate, particle or composition, e.g., a polymer-ZK-EPO conjugate, particle or composition described herein, e.g., a polymer-ZK-EPO conjugate comprising a ZK-EPO molecule, coupled, e.g., via a linker, to a polymer described herein. In an embodiment, the polymer-agent conjugate comprises a ZK-EPO molecule, coupled via a linker shown in FIG. 1 to a polymer, e.g., a polymer described herein. In an embodiment, the polymer-agent conjugate is a polymer-ZK-EPO conjugate shown in FIG. 1. In one embodiment, the polymer-ZK-EPO conjugate, particle or composition is administered at a dose and/or dosing schedule described herein.

In one aspect, the disclosure features a method of treating advanced non small cell lung cancer or small cell lung cancer in a subject, e.g., a human. The method comprises: administering a polymer-agent conjugate, particle or composition e.g., a polymer-agent conjugate, particle or composition described herein, to a subject in an amount effective to treat the cancer, to thereby treat the cancer.

In an embodiment, the polymer-agent conjugate comprises an anticancer agent such as an epothilone, coupled, e.g., via a linker, to a polymer described herein. In an embodiment, the polymer-agent conjugate comprises an anticancer agent such as an epothilone, coupled via a linker shown in FIG. 1 to a polymer, e.g., a polymer described herein. In an embodiment, the polymer-agent conjugate is a polymer-epothilone conjugate shown in FIG. 1.

In one embodiment, the polymer-agent conjugate, particle or composition is not administered in combination with a taxane.

In one embodiment, the polymer-agent conjugate, particle or composition is administered in combination with a vascular endothelial (VEGF) pathway inhibitor, e.g., a VEGF inhibitor or VEGF receptor inhibitor. In one embodiment, the VEGF inhibitor is bevacizumab. In another embodiment, the VEGF receptor inhibitor is selected from CP-547632 and AZD2171.

In one embodiment, the polymer-agent conjugate, particle or composition is administered in combination with an epidermal growth factor (EGF) pathway inhibitor, e.g., an EGF inhibitor or EGF receptor inhibitor. In one embodiment, the EGF receptor inhibitor is cetuximab, erlotinib, or gefitinib.

In one embodiment, the polymer-agent conjugate, particle or composition is administered in combination with a platinum-based agent (e.g., cisplatin, carboplatin, oxaliplatin). In one embodiment, the polymer-agent conjugate, particle or composition is administered in combination with a platinum-based agent (e.g., cisplatin, carboplatin, oxaliplatin) and a nucleoside analog (e.g., gemcitabine). In one embodiment, the polymer-agent conjugate, particle or composition is administered in combination with a platinum-based agent (e.g., cisplatin, carboplatin, oxaliplatin) and an anti-metabolite, e.g., an antifolate (e.g., floxuridine, pemetrexed) or pyrimidine analogue (e.g., 5FU). In one embodiment, the polymer-agent conjugate, particle or composition is administered in combination with a platinum-based agent (e.g., cisplatin, carboplatin or oxaliplatin) and a vinca alkaloid (e.g., vinblastine, vincristine, vindesine or vinorelbine).

In one embodiment, the polymer-agent conjugate, particle or composition is administered in combination with an mTOR inhibitor, e.g., rapamycin, everolimus, AP23573, CCI-779 or SDZ-RAD.

In one embodiment, the polymer-agent conjugate, particle or composition, either alone or with any of the combinations described herein, is administered in combination with radiation.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the polymer-ixabepilone conjugate, particle or composition is administered at a dose and/or dosing schedule described herein.

In one embodiment, the polymer-agent conjugate, particle or composition includes a polymer-epothilone B conjugate, particle or composition, e.g., a polymer-epothilone B conjugate, particle or composition described herein, e.g., a polymer-epothilone B conjugate comprising an epothilone B molecule, coupled, e.g., via a linker, to a polymer described herein. In an embodiment, the polymer-agent conjugate comprises an epothilone B molecule, coupled via a linker shown in FIG. 1 to a polymer, e.g., a polymer described herein. In an embodiment, the polymer-agent conjugate is a polymer-epothilone B conjugate shown in FIG. 1. In one embodiment, the polymer-epothilone B conjugate, particle or composition is administered at a dose and/or dosing schedule described herein.

In one embodiment, the polymer-agent conjugate, particle or composition includes a polymer-epothilone D conjugate, particle or composition, e.g., a polymer-epothilone D conjugate, particle or composition described herein, e.g., a polymer-epothilone D conjugate comprising an epothilone D molecule, coupled, e.g., via a linker, to a polymer described herein. In an embodiment, the polymer-agent conjugate comprises an epothilone D molecule, coupled via a linker shown in FIG. 1 to a polymer, e.g., a polymer described herein. In an embodiment, the polymer-agent conjugate is a polymer-epothilone D conjugate shown in FIG. 1. In one embodiment, the polymer-epothilone D conjugate, particle or composition is administered at a dose and/or dosing schedule described herein.

In one embodiment, the polymer-agent conjugate, particle or composition includes a polymer-BMS310705 conjugate, particle or composition, e.g., a polymer-BMS310705 conjugate, particle or composition described herein, e.g., a polymer-BMS310705 conjugate comprising a BMS310705 molecule, coupled, e.g., via a linker, to a polymer described herein. In an embodiment, the polymer-agent conjugate comprises a BMS310705 molecule, coupled via a linker shown in FIG. 1 to a polymer, e.g., a polymer described herein. In an embodiment, the polymer-agent conjugate is a polymer-BMS310705 conjugate shown in FIG. 1. In one embodiment, the polymer-BMS310705 conjugate, particle or composition is administered at a dose and/or dosing schedule described herein.

In one embodiment, the polymer-agent conjugate, particle or composition includes a polymer-dehydelone conjugate, particle or composition, e.g., a polymer-dehydelone conjugate, particle or composition described herein, e.g., a polymer-dehydelone conjugate comprising a dehydelone molecule, coupled, e.g., via a linker, to a polymer described herein. In an embodiment, the polymer-agent conjugate comprises a dehydelone molecule, coupled via a linker shown in FIG. 1 to a polymer, e.g., a polymer described herein. In an embodiment, the polymer-agent conjugate is a polymer-dehydelone conjugate shown in FIG. 1. In one embodiment, the polymer-dehydelone conjugate, particle or composition is administered at a dose and/or dosing schedule described herein.

In one embodiment, the polymer-agent conjugate, particle or composition includes a polymer-ZK-EPO conjugate, particle or composition, e.g., a polymer-ZK-EPO conjugate, particle or composition described herein, e.g., a polymer-ZK-EPO conjugate comprising a ZK-EPO molecule, coupled, e.g., via a linker, to a polymer described herein. In an embodiment, the polymer-agent conjugate comprises a ZK-EPO molecule, coupled via a linker shown in FIG. 1 to a polymer, e.g., a polymer described herein. In an embodiment, the polymer-agent conjugate is a polymer-ZK-EPO conjugate shown in FIG. 1. In one embodiment, the polymer-ZK-EPO conjugate, particle or composition is administered at a dose and/or dosing schedule described herein.

In one aspect, the disclosure features a method of treating advanced non small cell lung cancer or small cell lung cancer in a subject, e.g., a human. The method comprises:

providing a subject who has advanced non small cell lung cancer or small cell lung cancer and has been treated with a chemotherapeutic agent that did not effectively treat the cancer (e.g., the subject has a chemotherapeutic refractory, a chemotherapeutic resistant and/or a relapsed cancer) or who had an unacceptable side effect (e.g., the subject has a chemotherapeutic sensitive cancer); and

administering a polymer-agent conjugate, particle or composition, e.g., a polymer-agent conjugate, particle or composition described herein, to a subject in an amount effective to treat the cancer, to thereby treat the cancer.

In an embodiment, the polymer-agent conjugate comprises an anticancer agent such as an epothilone, coupled, e.g., via a linker, to a polymer described herein. In an embodiment, the polymer-agent conjugate comprises an anticancer agent such as an epothilone, coupled via a linker shown in FIG. 1 to a polymer, e.g., a polymer described herein. In an embodiment, the polymer-agent conjugate is a polymer-epothilone conjugate shown in FIG. 1.

In one embodiment, the subject has been treated with a taxane which did not effectively treat the cancer (e.g., the subject has a taxane refractory, a taxane resistant and/or a relapsed cancer).

In one embodiment, the subject has been treated with a vascular endothelial growth factor (VEGF) pathway inhibitor (e.g., a VEGF inhibitor or VEGF receptor inhibitor) which did not effectively treat the cancer (e.g., the subject has been treated with bevacizumab CP-547632 or AZD2171 which did not effectively treat the cancer).

In one embodiment, the subject has been treated with an endothelial growth factor (EGF) pathway inhibitor (e.g., an EGF inhibitor or an EGF receptor inhibitor) which did not effectively treat the cancer (e.g., the subject has been treated with cetuximab, erlotinib, gefitinib which did not effectively treat the cancer).

In one embodiment, the subject has been treated with a platinum-based agent which did not effectively treat the cancer (e.g., the subject has been treated with cisplatin, carboplatin or oxaliplatin which did not effectively treat the cancer).

In one embodiment, the polymer-agent conjugate, particle or composition is administered in combination with an anti-metabolite, e.g., an antifolate, e.g., floxuridine, pemetrexed or pyrimidine analogue (e.g., 5FU).

In one embodiment, the polymer-agent conjugate, particle or composition is administered in combination with an EGF pathway inhibitor, e.g., an EGF inhibitor or EGF receptor inhibitor. The EGF receptor inhibitor can be, e.g., cetuximab, erlotinib or gefitinib.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the polymer-ixabepilone conjugate, particle or composition is administered at a dose and/or dosing schedule described herein.

In one embodiment, the polymer-agent conjugate, particle or composition includes a polymer-epothilone B conjugate, particle or composition, e.g., a polymer-epothilone B conjugate, particle or composition described herein, e.g., a polymer-epothilone B conjugate comprising an epothilone B molecule, coupled, e.g., via a linker, to a polymer described herein. In an embodiment, the polymer-agent conjugate comprises an epothilone B molecule, coupled via a linker shown in FIG. 1 to a polymer, e.g., a polymer described herein. In an embodiment, the polymer-agent conjugate is a polymer-epothilone B conjugate shown in FIG. 1. In one embodiment, the polymer-epothilone B conjugate, particle or composition is administered at a dose and/or dosing schedule described herein.

In one embodiment, the polymer-agent conjugate, particle or composition includes a polymer-epothilone D conjugate, particle or composition, e.g., a polymer-epothilone D conjugate, particle or composition described herein, e.g., a polymer-epothilone D conjugate comprising an epothilone D molecule, coupled, e.g., via a linker, to a polymer described herein. In an embodiment, the polymer-agent conjugate comprises an epothilone D molecule, coupled via a linker shown in FIG. 1 to a polymer, e.g., a polymer described herein. In an embodiment, the polymer-agent conjugate is a polymer-epothilone D conjugate shown in FIG. 1. In one embodiment, the polymer-epothilone D conjugate, particle or composition is administered at a dose and/or dosing schedule described herein.

In one embodiment, the polymer-agent conjugate, particle or composition includes a polymer-BMS310705 conjugate, particle or composition, e.g., a polymer-BMS310705 conjugate, particle or composition described herein, e.g., a polymer-BMS310705 conjugate comprising a BMS310705 molecule, coupled, e.g., via a linker, to a polymer described herein. In an embodiment, the polymer-agent conjugate comprises a BMS310705 molecule, coupled via a linker shown in FIG. 1 to a polymer, e.g., a polymer described herein. In an embodiment, the polymer-agent conjugate is a polymer-BMS310705 conjugate shown in FIG. 1. In one embodiment, the polymer-BMS310705 conjugate, particle or composition is administered at a dose and/or dosing schedule described herein.

In one embodiment, the polymer-agent conjugate, particle or composition includes a polymer-dehydelone conjugate, particle or composition, e.g., a polymer-dehydelone conjugate, particle or composition described herein, e.g., a polymer-dehydelone conjugate comprising a dehydelone molecule, coupled, e.g., via a linker, to a polymer described herein. In an embodiment, the polymer-agent conjugate comprises a dehydelone molecule, coupled via a linker shown in FIG. 1 to a polymer, e.g., a polymer described herein. In an embodiment, the polymer-agent conjugate is a polymer-dehydelone conjugate shown in FIG. 1. In one embodiment, the polymer-dehydelone conjugate, particle or composition is administered at a dose and/or dosing schedule described herein.

In one embodiment, the polymer-agent conjugate, particle or composition includes a polymer-ZK-EPO conjugate, particle or composition, e.g., a polymer-ZK-EPO conjugate, particle or composition described herein, e.g., a polymer-ZK-EPO conjugate comprising a ZK-EPO molecule, coupled, e.g., via a linker, to a polymer described herein. In an embodiment, the polymer-agent conjugate comprises a ZK-EPO molecule, coupled via a linker shown in FIG. 1 to a polymer, e.g., a polymer described herein. In an embodiment, the polymer-agent conjugate is a polymer-ZK-EPO conjugate shown in FIG. 1. In one embodiment, the polymer-ZK-EPO conjugate, particle or composition is administered at a dose and/or dosing schedule described herein.

In one aspect, the disclosure features a method of treating advanced ovarian cancer (e.g., peritoneal or fallopian tube cancer) in a subject, e.g., a human. The method comprises: administering a polymer-agent conjugate, particle or composition, e.g., a polymer-agent conjugate, particle or composition described herein, to a subject in an amount effective to treat the cancer, to thereby treat the cancer.

In one embodiment, the polymer-agent conjugate, particle or composition is not administered in combination with a taxane.

In one embodiment, the polymer-agent conjugate, particle or composition is administered in combination with a taxane (e.g., paclitaxel, docetaxel).

In one embodiment, the polymer-agent conjugate, particle or composition is administered in combination with a platinum-based agent (e.g., cisplatin, carboplatin or oxaliplatin).

In an embodiment, the polymer-agent conjugate comprises an anticancer agent such as an epothilone, coupled, e.g., via a linker, to a polymer described herein. In an embodiment, the polymer-agent conjugate comprises an anticancer agent such as an epothilone, coupled via a linker shown in FIG. 1 to a polymer, e.g., a polymer described herein. In an embodiment, the polymer-agent conjugate is a polymer-epothilone conjugate shown in FIG. 1.

In one embodiment, the polymer-agent conjugate, particle or composition is administered in combination with one or more of: an anti-metabolite, e.g., an antifolate (e.g., pemetrexed, floxuridine, raltitrexed) or pyrimidine analog (e.g., capecitabine, cytarabine, gemcitabine, 5FU); an alkylating agent (e.g., cyclophosphamide, dacarbazine, melphalan, ifosfamide, temozolomide; a topoisomerase inhibitor (e.g., etoposide, topotecan, irinotecan, tenoposide, lamellarin D); a platinum based agent (carboplatin, cisplatin, oxaliplatin); a vinca alkaloid (e.g., vinblastine, vincristine, vindesine and vinorelbine). In one embodiment, the polymer-agent conjugate, particle or composition is administered in combination with one or more of: capecitabine, cyclophosphamide, etoposide, gemcitabine, ifosfamide, irinotecan, melphalan, oxaliplatin, vinorelbine, vincristine and pemetrexed.

In one embodiment, the polymer-agent conjugate, particle or composition is administered in combination with a vascular endothelial growth factor (VEGF) pathway inhibitor, e.g., a VEGF inhibitor or VEGF receptor inhibitor. In one embodiment, the VEGF inhibitor is bevacizumab. In another embodiment, the VEGF receptor inhibitor is selected from CP-547632 and AZD2171.

In one embodiment, the polymer-agent conjugate, particle or composition is administered in combination with an mTOR inhibitor, e.g., rapamycin, everolimus, AP23573, CCI-779 or SDZ-RAD.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the polymer-ixabepilone conjugate, particle or composition is administered at a dose and/or dosing schedule described herein.

In one embodiment, the polymer-agent conjugate, particle or composition includes a polymer-epothilone B conjugate, particle or composition, e.g., a polymer-epothilone B conjugate, particle or composition described herein, e.g., a polymer-epothilone B conjugate comprising an epothilone B molecule, coupled, e.g., via a linker, to a polymer described herein. In an embodiment, the polymer-agent conjugate comprises an epothilone B molecule, coupled via a linker shown in FIG. 1 to a polymer, e.g., a polymer described herein. In an embodiment, the polymer-agent conjugate is a polymer-epothilone B conjugate shown in FIG. 1. In one embodiment, the polymer-epothilone B conjugate, particle or composition is administered at a dose and/or dosing schedule described herein.

In one embodiment, the polymer-agent conjugate, particle or composition includes a polymer-epothilone D conjugate, particle or composition, e.g., a polymer-epothilone D conjugate, particle or composition described herein, e.g., a polymer-epothilone D conjugate comprising an epothilone D molecule, coupled, e.g., via a linker, to a polymer described herein. In an embodiment, the polymer-agent conjugate comprises an epothilone D molecule, coupled via a linker shown in FIG. 1 to a polymer, e.g., a polymer described herein. In an embodiment, the polymer-agent conjugate is a polymer-epothilone D conjugate shown in FIG. 1. In one embodiment, the polymer-epothilone D conjugate, particle or composition is administered at a dose and/or dosing schedule described herein.

In one embodiment, the polymer-agent conjugate, particle or composition includes a polymer-BMS310705 conjugate, particle or composition, e.g., a polymer-BMS310705 conjugate, particle or composition described herein, e.g., a polymer-BMS310705 conjugate comprising a BMS310705 molecule, coupled, e.g., via a linker, to a polymer described herein. In an embodiment, the polymer-agent conjugate comprises a BMS310705 molecule, coupled via a linker shown in FIG. 1 to a polymer, e.g., a polymer described herein. In an embodiment, the polymer-agent conjugate is a polymer-BMS310705 conjugate shown in FIG. 1. In one embodiment, the polymer-BMS310705 conjugate, particle or composition is administered at a dose and/or dosing schedule described herein.

In one embodiment, the polymer-agent conjugate, particle or composition includes a polymer-dehydelone conjugate, particle or composition, e.g., a polymer-dehydelone conjugate, particle or composition described herein, e.g., a polymer-dehydelone conjugate comprising a dehydelone molecule, coupled, e.g., via a linker, to a polymer described herein. In an embodiment, the polymer-agent conjugate comprises a dehydelone molecule, coupled via a linker shown in FIG. 1 to a polymer, e.g., a polymer described herein. In an embodiment, the polymer-agent conjugate is a polymer-dehydelone conjugate shown in FIG. 1. In one embodiment, the polymer-dehydelone conjugate, particle or composition is administered at a dose and/or dosing schedule described herein.

In one embodiment, the polymer-agent conjugate, particle or composition includes a polymer-ZK-EPO conjugate, particle or composition, e.g., a polymer-ZK-EPO conjugate, particle or composition described herein, e.g., a polymer-ZK-EPO conjugate comprising a ZK-EPO molecule, coupled, e.g., via a linker, to a polymer described herein. In an embodiment, the polymer-agent conjugate comprises a ZK-EPO molecule, coupled via a linker shown in FIG. 1 to a polymer, e.g., a polymer described herein. In an embodiment, the polymer-agent conjugate is a polymer-ZK-EPO conjugate shown in FIG. 1. In one embodiment, the polymer-ZK-EPO conjugate, particle or composition is administered at a dose and/or dosing schedule described herein.

In one aspect, the disclosure features a method of treating advanced ovarian cancer (e.g., peritoneal or fallopian tube cancer) in a subject, e.g., a human. The method comprises:

providing a subject who has advanced ovarian cancer and has been treated with a chemotherapeutic agent that did not effectively treat the cancer (e.g., the subject has a chemotherapeutic refractory, a chemotherapeutic resistant and/or a relapsed cancer) or who had an unacceptable side effect (e.g., the subject has a chemotherapeutic sensitive cancer); and

administering a polymer-agent conjugate, particle or composition, e.g., a polymer-agent conjugate, particle or composition described herein, to a subject in an amount effective to treat the cancer, to thereby treat the cancer.

In an embodiment, the polymer-agent conjugate comprises an anticancer agent such as an epothilone, coupled, e.g., via a linker, to a polymer described herein. In an embodiment, the polymer-agent conjugate comprises an anticancer agent such as an epothilone, coupled via a linker shown in FIG. 1 to a polymer, e.g., a polymer described herein. In an embodiment, the polymer-agent conjugate is a polymer-epothilone conjugate shown in FIG. 1.

In one embodiment, the subject has been treated with a platinum-based agent that did not effectively treat the cancer (e.g., the subject has been treated with cisplatin, carboplatin or oxaliplatin which did not effectively treat the cancer). In one embodiment, the subject has been treated with cisplatin or carboplatin which did not effectively treat the cancer.

In one embodiment, the subject has been treated with a taxane that did not effectively treat the cancer (e.g., the subject has been treated with paclitaxel or docetaxel which did not effectively treat the cancer). In one embodiment, the subject has been treated with paclitaxel or docetaxel which did not effectively treat the cancer.

In one embodiment, the polymer-agent conjugate, particle or composition is administered in combination with a nucleoside analog, e.g., gemcitabine.

In one embodiment, the polymer-agent conjugate, particle or composition is administered in combination with a pyrimidine analog, e.g., capecitabine.

In one embodiment, the polymer-agent conjugate, particle or composition is administered in combination with a pyrimidine analog, e.g., capecitabine, and a nucleoside analog, e.g., gemcitabine.

In one embodiment, the polymer-agent conjugate, particle or composition is administered in combination with an anthracycline, e.g., daunorubicin, doxorubicin, epirubicin, valrubicin and idarubicin. In one embodiment, the anthracycline is doxorubicin, e.g., liposomal doxorubicin.

In one embodiment, the polymer-agent conjugate, particle or composition is administered in combination with a topoisomerase I inhibitor, e.g., irinotecan, topotecan, tenoposide, lamellarin D, camptothecin (e.g., IT-101). In one embodiment the topoisomerase I inhibitor is topotecan. In another embodiment, the topoisomerase I inhibitor is irinotecan or etoposide.

In one embodiment, the polymer-agent conjugate, particle or composition is administered in combination with one or more of: an anti-metabolite, e.g., an antifolate (e.g., pemetrexed, floxuridine, raltitrexed) or pyrimidine analog (e.g., capecitabine, cytarabine, gemcitabine, 5FU); an alkylating agent (e.g., cyclophosphamide, dacarbazine, melphalan, ifosfamide, temozolomide); a platinum based agent (carboplatin, cisplatin, oxaliplatin); and a vinca alkaloid (e.g., vinblastine, vincristine, vindesine and vinorelbine). In one embodiment, the polymer-agent conjugate, particle or composition is administered in combination with one or more of: capecitabine, cyclophosphamide, etoposide, gemcitabine, ifosfamide, irinotecan, melphalan, oxaliplatin, vinorelbine, vincristine and pemetrexed.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the polymer-ixabepilone conjugate, particle or composition is administered at a dose and/or dosing schedule described herein.

In one embodiment, the polymer-agent conjugate, particle or composition includes a polymer-epothilone B conjugate, particle or composition, e.g., a polymer-epothilone B conjugate, particle or composition described herein, e.g., a polymer-epothilone B conjugate comprising an epothilone B molecule, coupled, e.g., via a linker, to a polymer described herein. In an embodiment, the polymer-agent conjugate comprises an epothilone B molecule, coupled via a linker shown in FIG. 1 to a polymer, e.g., a polymer described herein. In an embodiment, the polymer-agent conjugate is a polymer-epothilone B conjugate shown in FIG. 1. In one embodiment, the polymer-epothilone B conjugate, particle or composition is administered at a dose and/or dosing schedule described herein.

In one embodiment, the polymer-agent conjugate, particle or composition includes a polymer-epothilone D conjugate, particle or composition, e.g., a polymer-epothilone D conjugate, particle or composition described herein, e.g., a polymer-epothilone D conjugate comprising an epothilone D molecule, coupled, e.g., via a linker, to a polymer described herein. In an embodiment, the polymer-agent conjugate comprises an epothilone D molecule, coupled via a linker shown in FIG. 1 to a polymer, e.g., a polymer described herein. In an embodiment, the polymer-agent conjugate is a polymer-epothilone D conjugate shown in FIG. 1. In one embodiment, the polymer-epothilone D conjugate, particle or composition is administered at a dose and/or dosing schedule described herein.

In one embodiment, the polymer-agent conjugate, particle or composition includes a polymer-BMS310705 conjugate, particle or composition, e.g., a polymer-BMS310705 conjugate, particle or composition described herein, e.g., a polymer-BMS310705 conjugate comprising a BMS310705 molecule, coupled, e.g., via a linker, to a polymer described herein. In an embodiment, the polymer-agent conjugate comprises a BMS310705 molecule, coupled via a linker shown in FIG. 1 to a polymer, e.g., a polymer described herein. In an embodiment, the polymer-agent conjugate is a polymer-BMS310705 conjugate shown in FIG. 1. In one embodiment, the polymer-BMS310705 conjugate, particle or composition is administered at a dose and/or dosing schedule described herein.

In one embodiment, the polymer-agent conjugate, particle or composition includes a polymer-dehydelone conjugate, particle or composition, e.g., a polymer-dehydelone conjugate, particle or composition described herein, e.g., a polymer-dehydelone conjugate comprising a dehydelone molecule, coupled, e.g., via a linker, to a polymer described herein. In an embodiment, the polymer-agent conjugate comprises a dehydelone molecule, coupled via a linker shown in FIG. 1 to a polymer, e.g., a polymer described herein. In an embodiment, the polymer-agent conjugate is a polymer-dehydelone conjugate shown in FIG. 1. In one embodiment, the polymer-dehydelone conjugate, particle or composition is administered at a dose and/or dosing schedule described herein.

In one embodiment, the polymer-agent conjugate, particle or composition includes a polymer-ZK-EPO conjugate, particle or composition, e.g., a polymer-ZK-EPO conjugate, particle or composition described herein, e.g., a polymer-ZK-EPO conjugate comprising a ZK-EPO molecule, coupled, e.g., via a linker, to a polymer described herein. In an embodiment, the polymer-agent conjugate comprises a ZK-EPO molecule, coupled via a linker shown in FIG. 1 to a polymer, e.g., a polymer described herein. In an embodiment, the polymer-agent conjugate is a polymer-ZK-EPO conjugate shown in FIG. 1. In one embodiment, the polymer-ZK-EPO conjugate, particle or composition is administered at a dose and/or dosing schedule described herein.

In one aspect, the disclosure features a method of treating advanced or metastatic melanoma in a subject, e.g., a human. The method comprises: administering a polymer-agent conjugate, particle or composition, e.g., a polymer-agent conjugate, particle or composition described herein, to a subject in an amount effective to treat the cancer, to thereby treat the cancer.

In an embodiment, the polymer-agent conjugate comprises an anticancer agent such as an epothilone, coupled, e.g., via a linker, to a polymer described herein. In an embodiment, the polymer-agent conjugate comprises an anticancer agent such as an epothilone, coupled via a linker shown in FIG. 1 to a polymer, e.g., a polymer described herein. In an embodiment, the polymer-agent conjugate is a polymer-epothilone conjugate shown in FIG. 1.

In one embodiment, the polymer-agent conjugate, particle or composition is not administered in combination with a taxane.

In one embodiment, the polymer-agent conjugate, particle or composition is administered in combination with a platinum-based agent (e.g., cisplatin, carboplatin, oxaliplatin). In one embodiment, the polymer-agent conjugate, particle or composition is administered in combination with a platinum-based agent (e.g., cisplatin, carboplatin, oxaliplatin) and a tetrazine, e.g., dacarbazine, mitozolomide or temozolomide. In one embodiment, the polymer-agent conjugate, particle or composition is administered in combination with cisplatin or carboplatin and dacarbazine or temozolomide.

In one embodiment, the polymer-agent conjugate, particle or composition is administered in combination with a tetrazine, e.g., dacarbazine, mitozolomide or temozolomide. In one embodiment, the tetrazine is dacarbazine or temozolomide.

In one embodiment, the polymer-agent conjugate, particle or composition is administered with interleukin-2.

In one embodiment, the polymer-agent conjugate, particle or composition is administered in combination with interferon.

In one embodiment, the polymer-agent conjugate, particle or composition is administered in combination with a vascular endothelial growth factor (VEGF) pathway inhibitor, e.g., a VEGF inhibitor or VEGF receptor inhibitor. In one embodiment, the VEGF inhibitor is bevacizumab. In one embodiment, the VEGF receptor inhibitor is selected from CP-547632 and AZD2171.

In one embodiment, the polymer-agent conjugate, particle or composition is administered in combination with an mTOR inhibitor. The mTOR inhibitor can be, e.g., rapamycin, everolimus, AP23573, CCI-779, SDZ-RAD.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the polymer-ixabepilone conjugate, particle or composition is administered at a dose and/or dosing schedule described herein.

In one embodiment, the polymer-agent conjugate, particle or composition includes a polymer-epothilone B conjugate, particle or composition, e.g., a polymer-epothilone B conjugate, particle or composition described herein, e.g., a polymer-epothilone B conjugate comprising an epothilone B molecule, coupled, e.g., via a linker, to a polymer described herein. In an embodiment, the polymer-agent conjugate comprises an epothilone B molecule, coupled via a linker shown in FIG. 1 to a polymer, e.g., a polymer described herein. In an embodiment, the polymer-agent conjugate is a polymer-epothilone B conjugate shown in FIG. 1. In one embodiment, the polymer-epothilone B conjugate, particle or composition is administered at a dose and/or dosing schedule described herein.

In one embodiment, the polymer-agent conjugate, particle or composition includes a polymer-epothilone D conjugate, particle or composition, e.g., a polymer-epothilone D conjugate, particle or composition described herein, e.g., a polymer-epothilone D conjugate comprising an epothilone D molecule, coupled, e.g., via a linker, to a polymer described herein. In an embodiment, the polymer-agent conjugate comprises an epothilone D molecule, coupled via a linker shown in FIG. 1 to a polymer, e.g., a polymer described herein. In an embodiment, the polymer-agent conjugate is a polymer-epothilone D conjugate shown in FIG. 1. In one embodiment, the polymer-epothilone D conjugate, particle or composition is administered at a dose and/or dosing schedule described herein.

In one embodiment, the polymer-agent conjugate, particle or composition includes a polymer-BMS310705 conjugate, particle or composition, e.g., a polymer-BMS310705 conjugate, particle or composition described herein, e.g., a polymer-BMS310705 conjugate comprising a BMS310705 molecule, coupled, e.g., via a linker, to a polymer described herein. In an embodiment, the polymer-agent conjugate comprises a BMS310705 molecule, coupled via a linker shown in FIG. 1 to a polymer, e.g., a polymer described herein. In an embodiment, the polymer-agent conjugate is a polymer-BMS310705 conjugate shown in FIG. 1. In one embodiment, the polymer-BMS310705 conjugate, particle or composition is administered at a dose and/or dosing schedule described herein.

In one embodiment, the polymer-agent conjugate, particle or composition includes a polymer-dehydelone conjugate, particle or composition, e.g., a polymer-dehydelone conjugate, particle or composition described herein, e.g., a polymer-dehydelone conjugate comprising a dehydelone molecule, coupled, e.g., via a linker, to a polymer described herein. In an embodiment, the polymer-agent conjugate comprises a dehydelone molecule, coupled via a linker shown in FIG. 1 to a polymer, e.g., a polymer described herein. In an embodiment, the polymer-agent conjugate is a polymer-dehydelone conjugate shown in FIG. 1. In one embodiment, the polymer-dehydelone conjugate, particle or composition is administered at a dose and/or dosing schedule described herein.

In one embodiment, the polymer-agent conjugate, particle or composition includes a polymer-ZK-EPO conjugate, particle or composition, e.g., a polymer-ZK-EPO conjugate, particle or composition described herein, e.g., a polymer-ZK-EPO conjugate comprising a ZK-EPO molecule, coupled, e.g., via a linker, to a polymer described herein. In an embodiment, the polymer-agent conjugate comprises a ZK-EPO molecule, coupled via a linker shown in FIG. 1 to a polymer, e.g., a polymer described herein. In an embodiment, the polymer-agent conjugate is a polymer-ZK-EPO conjugate shown in FIG. 1. In one embodiment, the polymer-ZK-EPO conjugate, particle or composition is administered at a dose and/or dosing schedule described herein.

In one aspect, the disclosure features a method of treating advanced or metastatic melanoma in a subject, e.g., a human, the method comprising:

providing a subject who has advanced or metastatic melanoma and has been treated with a chemotherapeutic agent that did not effectively treat the cancer (e.g., the subject has a chemotherapeutic refractory, a chemotherapeutic resistant and/or a relapsed cancer) or who had unacceptable side effects (e.g., the subject has a chemotherapeutic sensitive cancer); and

administering a polymer-agent conjugate, particle or composition, e.g., a polymer-agent conjugate, particle or composition described herein, to a subject in an amount effective to treat the cancer, to thereby treat the cancer.

In an embodiment, the polymer-agent conjugate comprises an anticancer agent such as an epothilone, coupled, e.g., via a linker, to a polymer described herein. In an embodiment, the polymer-agent conjugate comprises an anticancer agent such as an epothilone, coupled via a linker shown in FIG. 1 to a polymer, e.g., a polymer described herein. In an embodiment, the polymer-agent conjugate is a polymer-epothilone conjugate shown in FIG. 1.

In one embodiment, the subject has been treated with a platinum-based agent which did not effectively treat the cancer (e.g., the subject has been treated with cisplatin, carboplatin or oxaliplatin which did not effectively treat the cancer).

In one embodiment, the subject has been treated with a taxane which did not effectively treat the cancer (e.g., the subject has a taxane refractory, taxane resistant and/or relapsed cancer). In one embodiment, the taxane is paclitaxel.

In one embodiment, the subject has been treated with a tetrazine which did not effectively treat the cancer (e.g., the subject has a dacarbazine, mitozolomide or temozolomide refractory, a dacarbazine, mitozolomide or temozolomide resistant and/or relapsed cancer).

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the polymer-ixabepilone conjugate, particle or composition is administered at a dose and/or dosing schedule described herein.

In one embodiment, the polymer-agent conjugate, particle or composition includes a polymer-epothilone B conjugate, particle or composition, e.g., a polymer-epothilone B conjugate, particle or composition described herein, e.g., a polymer-epothilone B conjugate comprising an epothilone B molecule, coupled, e.g., via a linker, to a polymer described herein. In an embodiment, the polymer-agent conjugate comprises an epothilone B molecule, coupled via a linker shown in FIG. 1 to a polymer, e.g., a polymer described herein. In an embodiment, the polymer-agent conjugate is a polymer-epothilone B conjugate shown in FIG. 1. In one embodiment, the polymer-epothilone B conjugate, particle or composition is administered at a dose and/or dosing schedule described herein.

In one embodiment, the polymer-agent conjugate, particle or composition includes a polymer-epothilone D conjugate, particle or composition, e.g., a polymer-epothilone D conjugate, particle or composition described herein, e.g., a polymer-epothilone D conjugate comprising an epothilone D molecule, coupled, e.g., via a linker, to a polymer described herein. In an embodiment, the polymer-agent conjugate comprises an epothilone D molecule, coupled via a linker shown in FIG. 1 to a polymer, e.g., a polymer described herein. In an embodiment, the polymer-agent conjugate is a polymer-epothilone D conjugate shown in FIG. 1. In one embodiment, the polymer-epothilone D conjugate, particle or composition is administered at a dose and/or dosing schedule described herein.

In one embodiment, the polymer-agent conjugate, particle or composition includes a polymer-BMS310705 conjugate, particle or composition, e.g., a polymer-BMS310705 conjugate, particle or composition described herein, e.g., a polymer-BMS310705 conjugate comprising a BMS310705 molecule, coupled, e.g., via a linker, to a polymer described herein. In an embodiment, the polymer-agent conjugate comprises a BMS310705 molecule, coupled via a linker shown in FIG. 1 to a polymer, e.g., a polymer described herein. In an embodiment, the polymer-agent conjugate is a polymer-BMS310705 conjugate shown in FIG. 1. In one embodiment, the polymer-BMS310705 conjugate, particle or composition is administered at a dose and/or dosing schedule described herein.

In one embodiment, the polymer-agent conjugate, particle or composition includes a polymer-dehydelone conjugate, particle or composition, e.g., a polymer-dehydelone conjugate, particle or composition described herein, e.g., a polymer-dehydelone conjugate comprising a dehydelone molecule, coupled, e.g., via a linker, to a polymer described herein. In an embodiment, the polymer-agent conjugate comprises a dehydelone molecule, coupled via a linker shown in FIG. 1 to a polymer, e.g., a polymer described herein. In an embodiment, the polymer-agent conjugate is a polymer-dehydelone conjugate shown in FIG. 1. In one embodiment, the polymer-dehydelone conjugate, particle or composition is administered at a dose and/or dosing schedule described herein.

In one embodiment, the polymer-agent conjugate, particle or composition includes a polymer-ZK-EPO conjugate, particle or composition, e.g., a polymer-ZK-EPO conjugate, particle or composition described herein, e.g., a polymer-ZK-EPO conjugate comprising a ZK-EPO molecule, coupled, e.g., via a linker, to a polymer described herein. In an embodiment, the polymer-agent conjugate comprises a ZK-EPO molecule, coupled via a linker shown in FIG. 1 to a polymer, e.g., a polymer described herein. In an embodiment, the polymer-agent conjugate is a polymer-ZK-EPO conjugate shown in FIG. 1. In one embodiment, the polymer-ZK-EPO conjugate, particle or composition is administered at a dose and/or dosing schedule described herein.

In one aspect, the disclosure features a method of treating advanced or metastatic colorectal cancer in a subject, e.g., a human. The method comprises: administering a polymer-agent conjugate, particle or composition, e.g., a polymer-agent conjugate, particle or composition described herein, to a subject in an amount effective to treat the cancer, to thereby treat the cancer.

In an embodiment, the polymer-agent conjugate comprises an anticancer agent such as an epothilone, coupled, e.g., via a linker, to a polymer described herein. In an embodiment, the polymer-agent conjugate comprises an anticancer agent such as an epothilone, coupled via a linker shown in FIG. 1 to a polymer, e.g., a polymer described herein. In an embodiment, the polymer-agent conjugate is a polymer-epothilone conjugate shown in FIG. 1.

In one embodiment, the polymer-agent conjugate, particle or composition is not administered in combination with a taxane.

In one embodiment, the polymer-agent conjugate, particle or composition is administered in combination with an antimetabolite, e.g., an antifolate (e.g., pemetrexed, raltitrexed). In one embodiment, the polymer-agent conjugate, particle or composition is administered in combination with an antimetabolite, e.g., 5FU, and leucovorin. In one embodiment, the polymer-agent conjugate, particle or composition is further administered in combination with a platinum-based agent (e.g., cisplatin, carboplatin, oxaliplatin). For example, in one embodiment, the polymer-agent conjugate, particle or composition is administered in combination with an antimetabolite, e.g., 5FU, leucovorin, and a platinum-based agent, e.g., oxaliplatin. In another embodiment, the antimetabolite is a pyrimidine analog, e.g., capecitabine.

In one embodiment, the polymer-agent conjugate, particle or composition is administered in combination with a platinum-based agent (e.g., cisplatin, carboplatin, oxaliplatin).

In one embodiment, the polymer-agent conjugate, particle or composition is administered in combination with a vascular endothelial growth factor (VEGF) pathway inhibitor, e.g., a VEGF inhibitor or VEGF receptor inhibitor. In one embodiment, the VEGF inhibitor is bevacizumab. In one embodiment, the VEGF receptor inhibitor is selected from CP-547632 and AZD2171. In one embodiment, the polymer-agent conjugate, particle or composition is administered in combination with a VEGF pathway inhibitor, e.g., bevacizumab, and an antimetabolite, e.g., an antifolate (e.g., pemetrexed, raltitrexed) or pyrimidine analogue (e.g., 5FU). In one embodiment, the polymer-agent conjugate, particle or composition is administered with a VEGF pathway inhibitor, e.g., bevacizumab, an antimetabolite, e.g., a pyrimidine analogue (e.g., 5FU), and leucovorin. In another embodiment, the polymer-agent conjugate, particle or composition is administered with a VEGF pathway inhibitor, e.g., bevacizumab, an antimetabolite, e.g., a pyrimidine analogue (e.g., 5FU), leucovorin, a platinum-based agent (e.g., cisplatin, carboplatin, oxaliplatin) and/or a topoisomerase inhibitor (e.g., irinotecan, topotecan, etoposide, teniposide, lamellarin D, camptothecin (e.g., IT-101)). For example, in one embodiment, the polymer-agent conjugate, particle or composition is administered with the following combination: a VEGF pathway inhibitor, e.g., bevacizumab, an antimetabolite (e.g., 5FU), leucovorin and a platinum-based agent (e.g., oxaliplatin); a VEGF pathway inhibitor, e.g., bevacizumab, an antimetabolite (e.g., 5FU), leucovorin, a platinum-based agent (e.g., oxaliplatin) and a topoisomerase inhibitor (e.g., irinotecan); or a VEGF pathway inhibitor, e.g., bevacizumab, an antimetabolite (e.g., 5FU), leucovorin and a topoisomerase inhibitor (e.g., irinotecan).

In another embodiment, the polymer-agent conjugate, particle or composition is administered in combination with a VEGF pathway inhibitor, e.g., bevacizumab, and an antimetabolite wherein the antimetabolite is a pyrimidine analog, e.g., capecitabine. In one embodiment, the polymer-agent conjugate, particle or composition is further administered in combination with a platinum-based agent (e.g., cisplatin, carboplatin, oxaliplatin) or a topoisomerase inhibitor (e.g., irinotecan, topotecan, etoposide, teniposide, lamellarin D, camptothecin (e.g., IT-101)). For example, in one embodiment, the polymer-agent conjugate, particle or composition is administered with the following combination: a VEGF pathway inhibitor, e.g., bevacizumab, a pyrimidine analog, e.g., capecitabine, and a platinum-based agent (e.g., oxaliplatin); or a VEGF pathway inhibitor, e.g., bevacizumab, a pyrimidine analog, e.g., capecitabine, and a topoisomerase inhibitor (e.g., irinotecan).

In one embodiment, the polymer-agent conjugate, particle or composition is administered in combination with an epidermal growth factor (EGF) pathway inhibitor, e.g., an EGF inhibitor or EGF receptor inhibitor. The EGF receptor inhibitor can be, e.g., cetuximab, erlotinib, gefitinib, panitumumab. In one embodiment, the polymer-agent conjugate, particle or composition is administered in combination with an EGF pathway inhibitor, e.g., cetuximab or panitumumab, and a VEGF pathway inhibitor, e.g., bevacizumab.

In one embodiment, the polymer-agent conjugate, particle or composition is administered in combination with a topoisomerase inhibitor (e.g., irinotecan, topotecan, etoposide, teniposide, lamellarin D, camptothecin (e.g., IT-101)). In one embodiment, the polymer-agent conjugate, particle or composition is administered in combination with a topoisomerase inhibitor (e.g., irinotecan) and a VEGF pathway inhibitor, e.g., bevacizumab.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the polymer-ixabepilone conjugate, particle or composition is administered at a dose and/or dosing schedule described herein.

In one embodiment, the polymer-agent conjugate, particle or composition includes a polymer-epothilone B conjugate, particle or composition, e.g., a polymer-epothilone B conjugate, particle or composition described herein, e.g., a polymer-epothilone B conjugate comprising an epothilone B molecule, coupled, e.g., via a linker, to a polymer described herein. In an embodiment, the polymer-agent conjugate comprises an epothilone B molecule, coupled via a linker shown in FIG. 1 to a polymer, e.g., a polymer described herein. In an embodiment, the polymer-agent conjugate is a polymer-epothilone B conjugate shown in FIG. 1. In one embodiment, the polymer-epothilone B conjugate, particle or composition is administered at a dose and/or dosing schedule described herein.

In one embodiment, the polymer-agent conjugate, particle or composition includes a polymer-epothilone D conjugate, particle or composition, e.g., a polymer-epothilone D conjugate, particle or composition described herein, e.g., a polymer-epothilone D conjugate comprising an epothilone D molecule, coupled, e.g., via a linker, to a polymer described herein. In an embodiment, the polymer-agent conjugate comprises an epothilone D molecule, coupled via a linker shown in FIG. 1 to a polymer, e.g., a polymer described herein. In an embodiment, the polymer-agent conjugate is a polymer-epothilone D conjugate shown in FIG. 1. In one embodiment, the polymer-epothilone D conjugate, particle or composition is administered at a dose and/or dosing schedule described herein.

In one embodiment, the polymer-agent conjugate, particle or composition includes a polymer-BMS310705 conjugate, particle or composition, e.g., a polymer-BMS310705 conjugate, particle or composition described herein, e.g., a polymer-BMS310705 conjugate comprising a BMS310705 molecule, coupled, e.g., via a linker, to a polymer described herein. In an embodiment, the polymer-agent conjugate comprises a BMS310705 molecule, coupled via a linker shown in FIG. 1 to a polymer, e.g., a polymer described herein. In an embodiment, the polymer-agent conjugate is a polymer-BMS310705 conjugate shown in FIG. 1. In one embodiment, the polymer-BMS310705 conjugate, particle or composition is administered at a dose and/or dosing schedule described herein.

In one embodiment, the polymer-agent conjugate, particle or composition includes a polymer-dehydelone conjugate, particle or composition, e.g., a polymer-dehydelone conjugate, particle or composition described herein, e.g., a polymer-dehydelone conjugate comprising a dehydelone molecule, coupled, e.g., via a linker, to a polymer described herein. In an embodiment, the polymer-agent conjugate comprises a dehydelone molecule, coupled via a linker shown in FIG. 1 to a polymer, e.g., a polymer described herein. In an embodiment, the polymer-agent conjugate is a polymer-dehydelone conjugate shown in FIG. 1. In one embodiment, the polymer-dehydelone conjugate, particle or composition is administered at a dose and/or dosing schedule described herein.

In one embodiment, the polymer-agent conjugate, particle or composition includes a polymer-ZK-EPO conjugate, particle or composition, e.g., a polymer-ZK-EPO conjugate, particle or composition described herein, e.g., a polymer-ZK-EPO conjugate comprising a ZK-EPO molecule, coupled, e.g., via a linker, to a polymer described herein. In an embodiment, the polymer-agent conjugate comprises a ZK-EPO molecule, coupled via a linker shown in FIG. 1 to a polymer, e.g., a polymer described herein. In an embodiment, the polymer-agent conjugate is a polymer-ZK-EPO conjugate shown in FIG. 1. In one embodiment, the polymer-ZK-EPO conjugate, particle or composition is administered at a dose and/or dosing schedule described herein.

In one aspect, the disclosure features a method of treating advanced or metastatic colorectal cancer in a subject, e.g., a human, the method comprising:

providing a subject who has advanced or metastatic colorectal cancer and has been treated with a chemotherapeutic agent that did not effectively treat the cancer (e.g., the subject has a chemotherapeutic refractory, chemotherapeutic resistant and/or relapsed cancer) or who had unacceptable side effects (e.g., the subject has a chemotherapeutic sensitive cancer); and

administering a composition comprising a polymer-agent conjugate, particle or composition, e.g., a polymer-agent conjugate, particle or composition described herein, to a subject in an amount effective to treat the cancer, to thereby treat the cancer.

In an embodiment, the polymer-agent conjugate comprises an anticancer agent such as an epothilone, coupled, e.g., via a linker, to a polymer described herein. In an embodiment, the polymer-agent conjugate comprises an anticancer agent such as an epothilone, coupled via a linker shown in FIG. 1 to a polymer, e.g., a polymer described herein. In an embodiment, the polymer-agent conjugate is a polymer-epothilone conjugate shown in FIG. 1.

In one embodiment, the subject has been treated with an anti-metabolite, e.g., a pyrimidine analogue which did not effectively treat the cancer (e.g., the subject has a capecitabine and/or 5FU refractory, a capecitabine and/or 5FU resistant and/or relapsed cancer).

In one embodiment, the subject has been treated with a pyrimidine analog which did not effectively treat the cancer (e.g., the subject has a capecitabine refractory, a capecitabine resistant and/or relapsed cancer).

In one embodiment, the polymer-agent conjugate, particle or composition is administered in combination with a vascular endothelial growth factor (VEGF) pathway inhibitor, e.g., a VEGF inhibitor or VEGF receptor inhibitor. In one embodiment, the VEGF inhibitor is bevacizumab. In one embodiment, the VEGF receptor inhibitor is selected from CP-547632 and AZD2171. In one embodiment, the polymer-agent conjugate, particle or composition is administered in combination with a VEGF pathway inhibitor, e.g., bevacizumab, and an antimetabolite, e.g., an antifolate (e.g., pemetrexed, raltitrexed) or pyrimidine analogue (e.g., 5FU). In one embodiment, the polymer-agent conjugate, particle or composition is administered with a VEGF pathway inhibitor, e.g., bevacizumab, an antimetabolite (e.g., 5FU) and leucovorin. In another embodiment, the polymer-agent conjugate, particle or composition is administered with a VEGF pathway inhibitor, e.g., bevacizumab, an antimetabolite (e.g., 5FU), leucovorin, a platinum-based agent (e.g., cisplatin, carboplatin, oxaliplatin) and/or a topoisomerase inhibitor (e.g., irinotecan, topotecan, etoposide, teniposide, lamellarin D, camptothecin (e.g., IT-101)). For example, in one embodiment, the polymer-agent conjugate, particle or composition is administered with the following combination: a VEGF pathway inhibitor, e.g., bevacizumab, an antimetabolite (e.g., 5FU), leucovorin and a platinum-based agent (e.g., oxaliplatin); a VEGF pathway inhibitor, e.g., bevacizumab, an antimetabolite (e.g., 5FU), leucovorin, a platinum-based agent (e.g., oxaliplatin) and a topoisomerase inhibitor (e.g., irinotecan); or a VEGF pathway inhibitor, e.g., bevacizumab, an antimetabolite (e.g., 5FU), leucovorin and a topoisomerase inhibitor (e.g., irinotecan).

In another embodiment, the polymer-agent conjugate, particle or composition is administered in combination with a VEGF pathway inhibitor, e.g., bevacizumab, and an antimetabolite wherein the antimetabolite is a pyrimidine analog, e.g., capecitabine. In one embodiment, the polymer-agent conjugate, particle or composition is further administered in combination with a platinum-based agent (e.g., cisplatin, carboplatin, oxaliplatin) or a topoisomerase inhibitor (e.g., irinotecan, topotecan, etoposide, teniposide, lamellarin D, camptothecin (e.g., IT-101)). For example, in one embodiment, the polymer-agent conjugate, particle or composition is administered with the following combination: a VEGF pathway inhibitor, e.g., bevacizumab, a pyrimidine analog, e.g., capecitabine, and a platinum-based agent (e.g., oxaliplatin); or a VEGF pathway inhibitor, e.g., bevacizumab, a pyrimidine analog, e.g., capecitabine, and a topoisomerase inhibitor (e.g., irinotecan).

In one embodiment, the polymer-agent conjugate, particle or composition is administered in combination with an epidermal growth factor (EGF) pathway inhibitor, e.g., an EGF inhibitor or EGF receptor inhibitor. The EGF receptor inhibitor can be, e.g., cetuximab, erlotinib, gefitinib, panitumumab. In one embodiment, the polymer-agent conjugate, particle or composition is administered in combination with an EGF pathway inhibitor, e.g., cetuximab or panitumumab, and a VEGF pathway inhibitor, e.g., bevacizumab.

In one embodiment, the polymer-agent conjugate, particle or composition is administered in combination with a topoisomerase inhibitor (e.g., irinotecan, topotecan, etoposide, teniposide, lamellarin D, camptothecin (e.g., IT-101)). In one embodiment, the polymer-agent conjugate, particle or composition is administered in combination with a topoisomerase inhibitor (e.g., irinotecan) and a VEGF pathway inhibitor, e.g., bevacizumab.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the polymer-ixabepilone conjugate, particle or composition is administered at a dose and/or dosing schedule described herein.

In one embodiment, the polymer-agent conjugate, particle or composition includes a polymer-epothilone B conjugate, particle or composition, e.g., a polymer-epothilone B conjugate, particle or composition described herein, e.g., a polymer-epothilone B conjugate comprising an epothilone B molecule, coupled, e.g., via a linker, to a polymer described herein. In an embodiment, the polymer-agent conjugate comprises an epothilone B molecule, coupled via a linker shown in FIG. 1 to a polymer, e.g., a polymer described herein. In an embodiment, the polymer-agent conjugate is a polymer-epothilone B conjugate shown in FIG. 1. In one embodiment, the polymer-epothilone B conjugate, particle or composition is administered at a dose and/or dosing schedule described herein.

In one embodiment, the polymer-agent conjugate, particle or composition includes a polymer-epothilone D conjugate, particle or composition, e.g., a polymer-epothilone D conjugate, particle or composition described herein, e.g., a polymer-epothilone D conjugate comprising an epothilone D molecule, coupled, e.g., via a linker, to a polymer described herein. In an embodiment, the polymer-agent conjugate comprises an epothilone D molecule, coupled via a linker shown in FIG. 1 to a polymer, e.g., a polymer described herein. In an embodiment, the polymer-agent conjugate is a polymer-epothilone D conjugate shown in FIG. 1. In one embodiment, the polymer-epothilone D conjugate, particle or composition is administered at a dose and/or dosing schedule described herein.

In one embodiment, the polymer-agent conjugate, particle or composition includes a polymer-BMS310705 conjugate, particle or composition, e.g., a polymer-BMS310705 conjugate, particle or composition described herein, e.g., a polymer-BMS310705 conjugate comprising a BMS310705 molecule, coupled, e.g., via a linker, to a polymer described herein. In an embodiment, the polymer-agent conjugate comprises a BMS310705 molecule, coupled via a linker shown in FIG. 1 to a polymer, e.g., a polymer described herein. In an embodiment, the polymer-agent conjugate is a polymer-BMS310705 conjugate shown in FIG. 1. In one embodiment, the polymer-BMS310705 conjugate, particle or composition is administered at a dose and/or dosing schedule described herein.

In one embodiment, the polymer-agent conjugate, particle or composition includes a polymer-dehydelone conjugate, particle or composition, e.g., a polymer-dehydelone conjugate, particle or composition described herein, e.g., a polymer-dehydelone conjugate comprising a dehydelone molecule, coupled, e.g., via a linker, to a polymer described herein. In an embodiment, the polymer-agent conjugate comprises a dehydelone molecule, coupled via a linker shown in FIG. 1 to a polymer, e.g., a polymer described herein. In an embodiment, the polymer-agent conjugate is a polymer-dehydelone conjugate shown in FIG. 1. In one embodiment, the polymer-dehydelone conjugate, particle or composition is administered at a dose and/or dosing schedule described herein.

In one embodiment, the polymer-agent conjugate, particle or composition includes a polymer-ZK-EPO conjugate, particle or composition, e.g., a polymer-ZK-EPO conjugate, particle or composition described herein, e.g., a polymer-ZK-EPO conjugate comprising a ZK-EPO molecule, coupled, e.g., via a linker, to a polymer described herein. In an embodiment, the polymer-agent conjugate comprises a ZK-EPO molecule, coupled via a linker shown in FIG. 1 to a polymer, e.g., a polymer described herein. In an embodiment, the polymer-agent conjugate is a polymer-ZK-EPO conjugate shown in FIG. 1. In one embodiment, the polymer-ZK-EPO conjugate, particle or composition is administered at a dose and/or dosing schedule described herein.

In one aspect, the disclosure features a method for selecting a subject, e.g., a human, with a proliferative disorder, e.g., cancer, for treatment with a polymer-agent conjugate, particle or composition, e.g., a polymer-agent conjugate, particle or composition described herein. The method comprises:

determining whether a subject with a proliferative disorder, e.g., cancer, has diabetes; and

selecting a subject for treatment with a polymer-agent conjugate, particle or composition, e.g., a polymer-ixabepilone conjugate, particle or composition described herein, on the basis that the subject has diabetes.

In an embodiment, the polymer-agent conjugate comprises an anticancer agent such as an epothilone, coupled, e.g., via a linker, to a polymer described herein. In an embodiment, the polymer-agent conjugate comprises an anticancer agent such as an epothilone, coupled via a linker shown in FIG. 1 to a polymer, e.g., a polymer described herein. In an embodiment, the polymer-agent conjugate is a polymer-epothilone conjugate shown in FIG. 1.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the subject is selected for treatment with the polymer-ixabepilone conjugate, particle or composition at a dose and/or dosing schedule described herein.

In one embodiment, the polymer-agent conjugate, particle or composition includes a polymer-epothilone B conjugate, particle or composition, e.g., a polymer-epothilone B conjugate, particle or composition described herein, e.g., a polymer-epothilone B conjugate comprising an epothilone B molecule, coupled, e.g., via a linker, to a polymer described herein. In an embodiment, the polymer-agent conjugate comprises an epothilone B molecule, coupled via a linker shown in FIG. 1 to a polymer, e.g., a polymer described herein. In an embodiment, the polymer-agent conjugate is a polymer-epothilone B conjugate shown in FIG. 1. In one embodiment, the subject is selected for treatment with the polymer-epothilone B conjugate, particle or composition at a dose and/or dosing schedule described herein.

In one embodiment, the polymer-agent conjugate, particle or composition includes a polymer-epothilone D conjugate, particle or composition, e.g., a polymer-epothilone D conjugate, particle or composition described herein, e.g., a polymer-epothilone D conjugate comprising an epothilone D molecule, coupled, e.g., via a linker, to a polymer described herein. In an embodiment, the polymer-agent conjugate comprises an epothilone D molecule, coupled via a linker shown in FIG. 1 to a polymer, e.g., a polymer described herein. In an embodiment, the polymer-agent conjugate is a polymer-epothilone D conjugate shown in FIG. 1. In one embodiment, the subject is selected for treatment with the polymer-epothilone D conjugate, particle or composition at a dose and/or dosing schedule described herein.

In one embodiment, the polymer-agent conjugate, particle or composition includes a polymer-BMS310705 conjugate, particle or composition, e.g., a polymer-BMS310705 conjugate, particle or composition described herein, e.g., a polymer-BMS310705 conjugate comprising a BMS310705 molecule, coupled, e.g., via a linker, to a polymer described herein. In an embodiment, the polymer-agent conjugate comprises a BMS310705 molecule, coupled via a linker shown in FIG. 1 to a polymer, e.g., a polymer described herein. In an embodiment, the polymer-agent conjugate is a polymer-BMS310705 conjugate shown in FIG. 1. In one embodiment, the subject is selected for treatment with the polymer-BMS310705 conjugate, particle or composition at a dose and/or dosing schedule described herein.

In one embodiment, the polymer-agent conjugate, particle or composition includes a polymer-dehydelone conjugate, particle or composition, e.g., a polymer-dehydelone conjugate, particle or composition described herein, e.g., a polymer-dehydelone conjugate comprising a dehydelone molecule, coupled, e.g., via a linker, to a polymer described herein. In an embodiment, the polymer-agent conjugate comprises a dehydelone molecule, coupled via a linker shown in FIG. 1 to a polymer, e.g., a polymer described herein. In an embodiment, the polymer-agent conjugate is a polymer-dehydelone conjugate shown in FIG. 1. In one embodiment, the subject is selected for treatment with the polymer-dehydelone conjugate, particle or composition at a dose and/or dosing schedule described herein.

In one embodiment, the polymer-agent conjugate, particle or composition includes a polymer-ZK-EPO conjugate, particle or composition, e.g., a polymer-ZK-EPO conjugate, particle or composition described herein, e.g., a polymer-ZK-EPO conjugate comprising a ZK-EPO molecule, coupled, e.g., via a linker, to a polymer described herein. In an embodiment, the polymer-agent conjugate comprises a ZK-EPO molecule, coupled via a linker shown in FIG. 1 to a polymer, e.g., a polymer described herein. In an embodiment, the polymer-agent conjugate is a polymer-ZK-EPO conjugate shown in FIG. 1. In one embodiment, the subject is selected for treatment with the polymer-ZK-EPO conjugate, particle or composition at a dose and/or dosing schedule described herein.

In one embodiment, the cancer is a cancer described herein. In one embodiment, the subject is selected for treatment with the polymer-agent conjugate, particle or composition in combination with one or more additional chemotherapeutic agent, e.g., a chemotherapeutic agent or combination of chemotherapeutic agents described herein.

In one aspect, the disclosure features a method for treating a subject, e.g., a human, with a proliferative disorder, e.g., cancer, comprising:

selecting a subject with a proliferative disorder who has diabetes; and

administering a polymer-agent conjugate, particle or composition, e.g., a polymer-agent conjugate, particle or composition described herein, to the subject in an amount effective to treat the disorder, to thereby treat the proliferative disorder.

In an embodiment, the polymer-agent conjugate comprises an anticancer agent such as an epothilone, coupled, e.g., via a linker, to a polymer described herein. In an embodiment, the polymer-agent conjugate comprises an anticancer agent such as an epothilone, coupled via a linker shown in FIG. 1 to a polymer, e.g., a polymer described herein. In an embodiment, the polymer-agent conjugate is a polymer-epothilone conjugate shown in FIG. 1.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the polymer-ixabepilone conjugate, particle or composition is administered at a dose and/or dosing schedule described herein.

In one embodiment, the polymer-agent conjugate, particle or composition includes a polymer-epothilone B conjugate, particle or composition, e.g., a polymer-epothilone B conjugate, particle or composition described herein, e.g., a polymer-epothilone B conjugate comprising an epothilone B molecule, coupled, e.g., via a linker, to a polymer described herein. In an embodiment, the polymer-agent conjugate comprises an epothilone B molecule, coupled via a linker shown in FIG. 1 to a polymer, e.g., a polymer described herein. In an embodiment, the polymer-agent conjugate is a polymer-epothilone B conjugate shown in FIG. 1. In one embodiment, the polymer-epothilone B conjugate, particle or composition is administered at a dose and/or dosing schedule described herein.

In one embodiment, the polymer-agent conjugate, particle or composition includes a polymer-epothilone D conjugate, particle or composition, e.g., a polymer-epothilone D conjugate, particle or composition described herein, e.g., a polymer-epothilone D conjugate comprising an epothilone D molecule, coupled, e.g., via a linker, to a polymer described herein. In an embodiment, the polymer-agent conjugate comprises an epothilone D molecule, coupled via a linker shown in FIG. 1 to a polymer, e.g., a polymer described herein. In an embodiment, the polymer-agent conjugate is a polymer-epothilone D conjugate shown in FIG. 1. In one embodiment, the polymer-epothilone D conjugate, particle or composition is administered at a dose and/or dosing schedule described herein.

In one embodiment, the polymer-agent conjugate, particle or composition includes a polymer-BMS310705 conjugate, particle or composition, e.g., a polymer-BMS310705 conjugate, particle or composition described herein, e.g., a polymer-BMS310705 conjugate comprising a BMS310705 molecule, coupled, e.g., via a linker, to a polymer described herein. In an embodiment, the polymer-agent conjugate comprises a BMS310705 molecule, coupled via a linker shown in FIG. 1 to a polymer, e.g., a polymer described herein. In an embodiment, the polymer-agent conjugate is a polymer-BMS310705 conjugate shown in FIG. 1. In one embodiment, the polymer-BMS310705 conjugate, particle or composition is administered at a dose and/or dosing schedule described herein.

In one embodiment, the polymer-agent conjugate, particle or composition includes a polymer-dehydelone conjugate, particle or composition, e.g., a polymer-dehydelone conjugate, particle or composition described herein, e.g., a polymer-dehydelone conjugate comprising a dehydelone molecule, coupled, e.g., via a linker, to a polymer described herein. In an embodiment, the polymer-agent conjugate comprises a dehydelone molecule, coupled via a linker shown in FIG. 1 to a polymer, e.g., a polymer described herein. In an embodiment, the polymer-agent conjugate is a polymer-dehydelone conjugate shown in FIG. 1. In one embodiment, the polymer-dehydelone conjugate, particle or composition is administered at a dose and/or dosing schedule described herein.

In one embodiment, the polymer-agent conjugate, particle or composition includes a polymer-ZK-EPO conjugate, particle or composition, e.g., a polymer-ZK-EPO conjugate, particle or composition described herein, e.g., a polymer-ZK-EPO conjugate comprising a ZK-EPO molecule, coupled, e.g., via a linker, to a polymer described herein. In an embodiment, the polymer-agent conjugate comprises a ZK-EPO molecule, coupled via a linker shown in FIG. 1 to a polymer, e.g., a polymer described herein. In an embodiment, the polymer-agent conjugate is a polymer-ZK-EPO conjugate shown in FIG. 1. In one embodiment, the polymer-ZK-EPO conjugate, particle or composition is administered at a dose and/or dosing schedule described herein.

In one embodiment, the cancer is a cancer described herein. In one embodiment, the polymer-agent conjugate, particle or composition is administered in combination with one or more additional chemotherapeutic agent, e.g., a chemotherapeutic agent or combination of chemotherapeutic agents described herein.

In one aspect, the disclosure features a method for selecting a subject, e.g., a human, with a proliferative disorder, e.g., cancer, for treatment with a polymer-agent conjugate, particle or composition, e.g., a polymer-agent conjugate, particle or composition described herein, comprising:

determining whether a subject with a proliferative disorder, e.g., cancer, has experienced neuropathy from treatment with a chemotherapeutic agent, e.g., a taxane, a vinca alkaloid, a platinum-based agent or an epothilone; and

selecting a subject for treatment with a polymer-agent conjugate, particle or composition, e.g., a polymer-agent conjugate, particle or composition described herein, on the basis that the subject has experienced neuropathy from treatment with a chemotherapeutic agent, e.g., a taxane, a vinca alkaloid, a platinum-based agent or an epothilone.

In an embodiment, the polymer-agent conjugate comprises an anticancer agent such as an epothilone, coupled, e.g., via a linker, to a polymer described herein. In an embodiment, the polymer-agent conjugate comprises an anticancer agent such as an epothilone, coupled via a linker shown in FIG. 1 to a polymer, e.g., a polymer described herein. In an embodiment, the polymer-agent conjugate is a polymer-epothilone conjugate shown in FIG. 1.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the subject is selected for treatment with the polymer-ixabepilone conjugate, particle or composition at a dose and/or dosing schedule described herein.

In one embodiment, the polymer-agent conjugate, particle or composition includes a polymer-epothilone B conjugate, particle or composition, e.g., a polymer-epothilone B conjugate, particle or composition described herein, e.g., a polymer-epothilone B conjugate comprising an epothilone B molecule, coupled, e.g., via a linker, to a polymer described herein. In an embodiment, the polymer-agent conjugate comprises an epothilone B molecule, coupled via a linker shown in FIG. 1 to a polymer, e.g., a polymer described herein. In an embodiment, the polymer-agent conjugate is a polymer-epothilone B conjugate shown in FIG. 1. In one embodiment, the subject is selected for treatment with the polymer-epothilone B conjugate, particle or composition at a dose and/or dosing schedule described herein.

In one embodiment, the polymer-agent conjugate, particle or composition includes a polymer-epothilone D conjugate, particle or composition, e.g., a polymer-epothilone D conjugate, particle or composition described herein, e.g., a polymer-epothilone D conjugate comprising an epothilone D molecule, coupled, e.g., via a linker, to a polymer described herein. In an embodiment, the polymer-agent conjugate comprises an epothilone D molecule, coupled via a linker shown in FIG. 1 to a polymer, e.g., a polymer described herein. In an embodiment, the polymer-agent conjugate is a polymer-epothilone D conjugate shown in FIG. 1. In one embodiment, the subject is selected for treatment with the polymer-epothilone D conjugate, particle or composition at a dose and/or dosing schedule described herein.

In one embodiment, the polymer-agent conjugate, particle or composition includes a polymer-BMS310705 conjugate, particle or composition, e.g., a polymer-BMS310705 conjugate, particle or composition described herein, e.g., a polymer-BMS310705 conjugate comprising a BMS310705 molecule, coupled, e.g., via a linker, to a polymer described herein. In an embodiment, the polymer-agent conjugate comprises a BMS310705 molecule, coupled via a linker shown in FIG. 1 to a polymer, e.g., a polymer described herein. In an embodiment, the polymer-agent conjugate is a polymer-BMS310705 conjugate shown in FIG. 1. In one embodiment, the subject is selected for treatment with the polymer-BMS310705 conjugate, particle or composition at a dose and/or dosing schedule described herein.

In one embodiment, the polymer-agent conjugate, particle or composition includes a polymer-dehydelone conjugate, particle or composition, e.g., a polymer-dehydelone conjugate, particle or composition described herein, e.g., a polymer-dehydelone conjugate comprising a dehydelone molecule, coupled, e.g., via a linker, to a polymer described herein. In an embodiment, the polymer-agent conjugate comprises a dehydelone molecule, coupled via a linker shown in FIG. 1 to a polymer, e.g., a polymer described herein. In an embodiment, the polymer-agent conjugate is a polymer-dehydelone conjugate shown in FIG. 1. In one embodiment, the subject is selected for treatment with the polymer-dehydelone conjugate, particle or composition at a dose and/or dosing schedule described herein.

In one embodiment, the polymer-agent conjugate, particle or composition includes a polymer-ZK-EPO conjugate, particle or composition, e.g., a polymer-ZK-EPO conjugate, particle or composition described herein, e.g., a polymer-ZK-EPO conjugate comprising a ZK-EPO molecule, coupled, e.g., via a linker, to a polymer described herein. In an embodiment, the polymer-agent conjugate comprises a ZK-EPO molecule, coupled via a linker shown in FIG. 1 to a polymer, e.g., a polymer described herein. In an embodiment, the polymer-agent conjugate is a polymer-ZK-EPO conjugate shown in FIG. 1. In one embodiment, the subject is selected for treatment with the polymer-ZK-EPO conjugate, particle or composition at a dose and/or dosing schedule described herein.

In one embodiment, the neuropathy is peripheral neuropathy. In one embodiment, the neuropathy is sensory neuropathy, motor neuropathy or both. In one embodiment, the neuropathy is central nervous system neuropathy.

In one embodiment, the cancer is a cancer described herein. In one embodiment, the subject is selected for treatment with the polymer-agent conjugate, particle or composition in combination with one or more additional chemotherapeutic agent, e.g., a chemotherapeutic agent or combination of chemotherapeutic agents described herein.

In one aspect, the disclosure features a method for treating a subject, e.g., a human, with a proliferative disorder, e.g., cancer, comprising:

selecting a subject with a proliferative disorder, e.g., cancer, who has experienced one or more symptom of neuropathy from treatment with a chemotherapeutic agent, e.g., a taxane, a vinca alkaloid, a platinum-based agent or an epothilone; and

administering a polymer-agent conjugate, particle or composition, e.g., a polymer-agent conjugate, particle or composition described herein, to the subject in an amount effective to treat the disorder, to thereby treat the proliferative disorder.

In an embodiment, the polymer-agent conjugate comprises an anticancer agent such as an epothilone, coupled, e.g., via a linker, to a polymer described herein. In an embodiment, the polymer-agent conjugate comprises an anticancer agent such as an epothilone, coupled via a linker shown in FIG. 1 to a polymer, e.g., a polymer described herein. In an embodiment, the polymer-agent conjugate is a polymer-epothilone conjugate shown in FIG. 1.

In one embodiment, the subject experienced moderate to severe neuropathy from treatment with an epothilone. In one embodiment, the neuropathy is peripheral neuropathy. In one embodiment, the neuropathy is sensory neuropathy, motor neuropathy or both. In one embodiment, the neuropathy is central nervous system neuropathy.

In one embodiment, the subject has experienced neuropathy after two, three fours, five cycles of treatment with an epothilone.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the polymer-ixabepilone conjugate, particle or composition is administered at a dose and/or dosing schedule described herein.

In one embodiment, the polymer-agent conjugate, particle or composition includes a polymer-epothilone B conjugate, particle or composition, e.g., a polymer-epothilone B conjugate, particle or composition described herein, e.g., a polymer-epothilone B conjugate comprising an epothilone B molecule, coupled, e.g., via a linker, to a polymer described herein. In an embodiment, the polymer-agent conjugate comprises an epothilone B molecule, coupled via a linker shown in FIG. 1 to a polymer, e.g., a polymer described herein. In an embodiment, the polymer-agent conjugate is a polymer-epothilone B conjugate shown in FIG. 1. In one embodiment, the polymer-epothilone B conjugate, particle or composition is administered at a dose and/or dosing schedule described herein.

In one embodiment, the polymer-agent conjugate, particle or composition includes a polymer-epothilone D conjugate, particle or composition, e.g., a polymer-epothilone D conjugate, particle or composition described herein, e.g., a polymer-epothilone D conjugate comprising an epothilone D molecule, coupled, e.g., via a linker, to a polymer described herein. In an embodiment, the polymer-agent conjugate comprises an epothilone D molecule, coupled via a linker shown in FIG. 1 to a polymer, e.g., a polymer described herein. In an embodiment, the polymer-agent conjugate is a polymer-epothilone D conjugate shown in FIG. 1. In one embodiment, the polymer-epothilone D conjugate, particle or composition is administered at a dose and/or dosing schedule described herein.

In one embodiment, the polymer-agent conjugate, particle or composition includes a polymer-BMS310705 conjugate, particle or composition, e.g., a polymer-BMS310705 conjugate, particle or composition described herein, e.g., a polymer-BMS310705 conjugate comprising a BMS310705 molecule, coupled, e.g., via a linker, to a polymer described herein. In an embodiment, the polymer-agent conjugate comprises a BMS310705 molecule, coupled via a linker shown in FIG. 1 to a polymer, e.g., a polymer described herein. In an embodiment, the polymer-agent conjugate is a polymer-BMS310705 conjugate shown in FIG. 1. In one embodiment, the polymer-BMS310705 conjugate, particle or composition is administered at a dose and/or dosing schedule described herein.

In one embodiment, the polymer-agent conjugate, particle or composition includes a polymer-dehydelone conjugate, particle or composition, e.g., a polymer-dehydelone conjugate, particle or composition described herein, e.g., a polymer-dehydelone conjugate comprising a dehydelone molecule, coupled, e.g., via a linker, to a polymer described herein. In an embodiment, the polymer-agent conjugate comprises a dehydelone molecule, coupled via a linker shown in FIG. 1 to a polymer, e.g., a polymer described herein. In an embodiment, the polymer-agent conjugate is a polymer-dehydelone conjugate shown in FIG. 1. In one embodiment, the polymer-dehydelone conjugate, particle or composition is administered at a dose and/or dosing schedule described herein.

In one embodiment, the polymer-agent conjugate, particle or composition includes a polymer-ZK-EPO conjugate, particle or composition, e.g., a polymer-ZK-EPO conjugate, particle or composition described herein, e.g., a polymer-ZK-EPO conjugate comprising polymer-a ZK-EPO molecule, coupled, e.g., via a linker, to a polymer described herein. In an embodiment, the polymer-agent conjugate comprises a ZK-EPO molecule, coupled via a linker shown in FIG. 1 to a polymer, e.g., a polymer described herein. In an embodiment, the polymer-agent conjugate is a polymer-ZK-EPO conjugate shown in FIG. 1. In one embodiment, the polymer-ZK-EPO conjugate, particle or composition is administered at a dose and/or dosing schedule described herein.

In one embodiment, the cancer is a cancer described herein. In one embodiment, the polymer-agent conjugate, particle or composition is administered in combination with one or more additional chemotherapeutic agent, e.g., a chemotherapeutic agent or combination of chemotherapeutic agents described herein.

In one aspect, the disclosure features a method for selecting a subject, e.g., a human, with a proliferative disorder, e.g., cancer, for treatment with a polymer-agent conjugate, particle or composition, e.g., a polymer-agent conjugate, particle or composition described herein, comprising:

determining whether a subject with a proliferative disorder has moderate to severe neuropathy; and

selecting a subject for treatment with a polymer-agent conjugate, particle or composition on the basis that the subject has moderate to severe neuropathy.

In an embodiment, the polymer-agent conjugate comprises an anticancer agent such as an epothilone, coupled, e.g., via a linker, to a polymer described herein. In an embodiment, the polymer-agent conjugate comprises an anticancer agent such as an epothilone, coupled via a linker shown in FIG. 1 to a polymer, e.g., a polymer described herein. In an embodiment, the polymer-agent conjugate is a polymer-epothilone conjugate shown in FIG. 1.

In one embodiment, the method further comprises administering a polymer-agent conjugate, particle or composition, e.g., a polymer-agent conjugate, particle or composition described herein, to the subject.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, a dose and/or dosing schedule described herein is selected for administration to the subject.

In one embodiment, the polymer-agent conjugate, particle or composition includes a polymer-epothilone B conjugate, particle or composition, e.g., a polymer-epothilone B conjugate, particle or composition described herein, e.g., a polymer-epothilone B conjugate comprising an epothilone B molecule, coupled, e.g., via a linker, to a polymer described herein. In an embodiment, the polymer-agent conjugate comprises an epothilone B molecule, coupled via a linker shown in FIG. 1 to a polymer, e.g., a polymer described herein. In an embodiment, the polymer-agent conjugate is a polymer-epothilone B conjugate shown in FIG. 1. In one embodiment, a dose and/or dosing schedule described herein is selected for administration to the subject.

In one embodiment, the polymer-agent conjugate, particle or composition includes a polymer-epothilone D conjugate, particle or composition, e.g., a polymer-epothilone D conjugate, particle or composition described herein, e.g., a polymer-epothilone D conjugate comprising an epothilone D molecule, coupled, e.g., via a linker, to a polymer described herein. In an embodiment, the polymer-agent conjugate comprises an epothilone D molecule, coupled via a linker shown in FIG. 1 to a polymer, e.g., a polymer described herein. In an embodiment, the polymer-agent conjugate is a polymer-epothilone D conjugate shown in FIG. 1. In one embodiment, a dose and/or dosing schedule described herein is selected for administration to the subject.

In one embodiment, the polymer-agent conjugate, particle or composition includes a polymer-BMS310705 conjugate, particle or composition, e.g., a polymer-BMS310705 conjugate, particle or composition described herein, e.g., a polymer-BMS310705 conjugate comprising a BMS310705 molecule, coupled, e.g., via a linker, to a polymer described herein. In an embodiment, the polymer-agent conjugate comprises a BMS310705 molecule, coupled via a linker shown in FIG. 1 to a polymer, e.g., a polymer described herein. In an embodiment, the polymer-agent conjugate is a polymer-BMS310705 conjugate shown in FIG. 1. In one embodiment, a dose and/or dosing schedule described herein is selected for administration to the subject.

In one embodiment, the polymer-agent conjugate, particle or composition includes a polymer-dehydelone conjugate, particle or composition, e.g., a polymer-dehydelone conjugate, particle or composition described herein, e.g., a polymer-dehydelone conjugate comprising a dehydelone molecule, coupled, e.g., via a linker, to a polymer described herein. In an embodiment, the polymer-agent conjugate comprises a dehydelone molecule, coupled via a linker shown in FIG. 1 to a polymer, e.g., a polymer described herein. In an embodiment, the polymer-agent conjugate is a polymer-dehydelone conjugate shown in FIG. 1. In one embodiment, a dose and/or dosing schedule described herein is selected for administration to the subject.

In one embodiment, the polymer-agent conjugate, particle or composition includes a polymer-ZK-EPO conjugate, particle or composition, e.g., a polymer-ZK-EPO conjugate, particle or composition described herein, e.g., a polymer-ZK-EPO conjugate comprising a ZK-EPO molecule, coupled, e.g., via a linker, to a polymer described herein. In an embodiment, the polymer-agent conjugate comprises a ZK-EPO molecule, coupled via a linker shown in FIG. 1 to a polymer, e.g., a polymer described herein. In an embodiment, the polymer-agent conjugate is a polymer-ZK-EPO conjugate shown in FIG. 1. In one embodiment, a dose and/or dosing schedule described herein is selected for administration to the subject.

In one embodiment, the subject experienced moderate to severe neuropathy from treatment with an epothilone. In one embodiment, the neuropathy is peripheral neuropathy. In one embodiment, the neuropathy is sensory neuropathy, motor neuropathy or both. In one embodiment, the neuropathy is central nervous system neuropathy.

In one embodiment, the cancer is a cancer described herein. In one embodiment, the polymer-agent conjugate, particle or composition is administered in combination with one or more additional chemotherapeutic agent, e.g., a chemotherapeutic agent or combination of chemotherapeutic agents described herein.

In one aspect, the disclosure features a method for treating a subject, e.g., a human, with a proliferative disorder, e.g., cancer, comprising:

selecting a subject with a proliferative disorder, e.g., cancer, who has moderate to severe neuropathy; and

administering a polymer-agent conjugate, particle or composition, e.g., a polymer-agent conjugate, particle or composition described herein, to the subject in an amount effective to treat the disorder, to thereby treat the proliferative disorder.

In an embodiment, the polymer-agent conjugate comprises an anticancer agent such as an epothilone, coupled, e.g., via a linker, to a polymer described herein. In an embodiment, the polymer-agent conjugate comprises an anticancer agent such as an epothilone, coupled via a linker shown in FIG. 1 to a polymer, e.g., a polymer described herein. In an embodiment, the polymer-agent conjugate is a polymer-epothilone conjugate shown in FIG. 1.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the polymer-ixabepilone conjugate, particle or composition is administered at a dose and/or dosing schedule described herein.

In one embodiment, the polymer-agent conjugate, particle or composition includes a polymer-epothilone B conjugate, particle or composition, e.g., a polymer-epothilone B conjugate, particle or composition described herein, e.g., a polymer-epothilone B conjugate comprising an epothilone B molecule, coupled, e.g., via a linker, to a polymer described herein. In an embodiment, the polymer-agent conjugate comprises an epothilone B molecule, coupled via a linker shown in FIG. 1 to a polymer, e.g., a polymer described herein. In an embodiment, the polymer-agent conjugate is a polymer-epothilone B conjugate shown in FIG. 1. In one embodiment, the polymer-epothilone B conjugate, particle or composition is administered at a dose and/or dosing schedule described herein.

In one embodiment, the polymer-agent conjugate, particle or composition includes a polymer-epothilone D conjugate, particle or composition, e.g., a polymer-epothilone D conjugate, particle or composition described herein, e.g., a polymer-epothilone D conjugate comprising an epothilone D molecule, coupled, e.g., via a linker, to a polymer described herein. In an embodiment, the polymer-agent conjugate comprises an epothilone D molecule, coupled via a linker shown in FIG. 1 to a polymer, e.g., a polymer described herein. In an embodiment, the polymer-agent conjugate is a polymer-epothilone D conjugate shown in FIG. 1. In one embodiment, the polymer-epothilone D conjugate, particle or composition is administered at a dose and/or dosing schedule described herein.

In one embodiment, the polymer-agent conjugate, particle or composition includes a polymer-BMS310705 conjugate, particle or composition, e.g., a polymer-BMS310705 conjugate, particle or composition described herein, e.g., a polymer-BMS310705 conjugate comprising a BMS310705 molecule, coupled, e.g., via a linker, to a polymer described herein. In an embodiment, the polymer-agent conjugate comprises a BMS310705 molecule, coupled via a linker shown in FIG. 1 to a polymer, e.g., a polymer described herein. In an embodiment, the polymer-agent conjugate is a polymer-BMS310705 conjugate shown in FIG. 1. In one embodiment, the polymer-BMS310705 conjugate, particle or composition is administered at a dose and/or dosing schedule described herein.

In one embodiment, the polymer-agent conjugate, particle or composition includes a polymer-dehydelone conjugate, particle or composition, e.g., a polymer-dehydelone conjugate, particle or composition described herein, e.g., a polymer-dehydelone conjugate comprising a dehydelone molecule, coupled, e.g., via a linker, to a polymer described herein. In an embodiment, the polymer-agent conjugate comprises a dehydelone molecule, coupled via a linker shown in FIG. 1 to a polymer, e.g., a polymer described herein. In an embodiment, the polymer-agent conjugate is a polymer-dehydelone conjugate shown in FIG. 1. In one embodiment, the polymer-dehydelone conjugate, particle or composition is administered at a dose and/or dosing schedule described herein.

In one embodiment, the polymer-agent conjugate, particle or composition includes a polymer-ZK-EPO conjugate, particle or composition, e.g., a polymer-ZK-EPO conjugate, particle or composition described herein, e.g., a polymer-ZK-EPO conjugate comprising a ZK-EPO molecule, coupled, e.g., via a linker, to a polymer described herein. In an embodiment, the polymer-agent conjugate comprises a ZK-EPO molecule, coupled via a linker shown in FIG. 1 to a polymer, e.g., a polymer described herein. In an embodiment, the polymer-agent conjugate is a polymer-ZK-EPO conjugate shown in FIG. 1. In one embodiment, the polymer-ZK-EPO conjugate, particle or composition is administered at a dose and/or dosing schedule described herein.

In one embodiment, the subject experienced moderate to severe neuropathy from treatment with an epothilone. In one embodiment, the neuropathy is peripheral neuropathy. In one embodiment, the neuropathy is sensory neuropathy, motor neuropathy or both. In one embodiment, the neuropathy is central nervous system neuropathy.

In one embodiment, the cancer is a cancer described herein. In one embodiment, the polymer-agent conjugate, particle or composition is administered in combination with one or more additional chemotherapeutic agent, e.g., a chemotherapeutic agent or combination of chemotherapeutic agents described herein.

In another aspect, the disclosure features a method for selecting a subject, e.g., a human, with a proliferative disorder, e.g., cancer, for treatment with a polymer-agent conjugate, particle or composition, e.g., a polymer-agent conjugate, particle or composition described herein, comprising:

determining whether a subject with a proliferative disorder, e.g., cancer, has experienced an infusion site reaction (e.g., during or within 12 hours of infusion of an epothilone (e.g., ixabepilone)) to treatment with an epothilone (e.g., ixabepilone); and

selecting a subject for treatment with a polymer-agent conjugate, particle or composition on the basis that the subject is in need of reduced infusion site reaction (e.g., reduced as compared to the reaction associated with or caused by the treatment with an epothilone (e.g., ixabepilone)).

In an embodiment, the polymer-agent conjugate comprises an anticancer agent such as an epothilone, coupled, e.g., via a linker, to a polymer described herein. In an embodiment, the polymer-agent conjugate comprises an epothilone molecule, coupled via a linker shown in FIG. 1 to a polymer, e.g., a polymer described herein. In an embodiment, the polymer-agent conjugate is a polymer-epothilone conjugate shown in FIG. 1.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the subject is selected for treatment with the polymer-ixabepilone conjugate, particle or composition at a dose and/or dosing schedule described herein.

In one embodiment, the polymer-agent conjugate, particle or composition includes a polymer-epothilone D conjugate, particle or composition, e.g., a polymer-epothilone D conjugate, particle or composition described herein, e.g., a polymer-epothilone D conjugate comprising an epothilone D molecule, coupled, e.g., via a linker, to a polymer described herein. In an embodiment, the polymer-agent conjugate comprises an epothilone D molecule, coupled via a linker shown in FIG. 1 to a polymer, e.g., a polymer described herein. In an embodiment, the polymer-agent conjugate is a polymer-epothilone D conjugate shown in FIG. 1. In one embodiment, the subject is selected for treatment with the polymer-epothilone D conjugate, particle or composition at a dose and/or dosing schedule described herein.

In one embodiment, the cancer is a cancer described herein. In one embodiment, the polymer-agent conjugate, particle or composition is selected for administration in combination with one or more additional chemotherapeutic agent, e.g., a chemotherapeutic agent or combination of chemotherapeutic agents described herein.

In one aspect, the disclosure features a method of treating a subject, e.g., a human, with a proliferative disorder, e.g., cancer, comprising:

selecting a subject with a proliferative disorder, e.g., cancer, who has experienced an infusion site reaction to treatment with an epothilone; and

administering a polymer-agent conjugate, particle or composition, e.g., a polymer-agent conjugate, particle or composition described herein, to the subject in an amount effective to treat the disorder, to thereby treat the proliferative disorder.

In an embodiment, the polymer-agent conjugate comprises an anticancer agent such as an epothilone, coupled, e.g., via a linker, to a polymer described herein. In an embodiment, the polymer-agent conjugate comprises an epothilone molecule, coupled via a linker shown in FIG. 1 to a polymer, e.g., a polymer described herein. In an embodiment, the polymer-agent conjugate is a polymer-epothilone conjugate shown in FIG. 1.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the subject is selected for treatment with the polymer-ixabepilone conjugate, particle or composition at a dose and/or dosing schedule described herein.

In one embodiment, the polymer-agent conjugate, particle or composition includes a polymer-epothilone D conjugate, particle or composition, e.g., a polymer-epothilone D conjugate, particle or composition described herein, e.g., a polymer-epothilone D conjugate comprising an epothilone D molecule, coupled, e.g., via a linker, to a polymer described herein. In an embodiment, the polymer-agent conjugate comprises an epothilone D molecule, coupled via a linker shown in FIG. 1 to a polymer, e.g., a polymer described herein. In an embodiment, the polymer-agent conjugate is a polymer-epothilone D conjugate shown in FIG. 1. In one embodiment, the subject is selected for treatment with the polymer-epothilone D conjugate, particle or composition at a dose and/or dosing schedule described herein.

In one embodiment, the cancer is a cancer described herein. In one embodiment, the polymer-agent conjugate, particle or composition is administered in combination with one or more additional chemotherapeutic agent, e.g., a chemotherapeutic agent or combination of chemotherapeutic agents described herein.

In one aspect, the disclosure features a method of treating a subject, e.g., a human, with a proliferative disorder, e.g., cancer, comprising:

administering a polymer-agent conjugate, particle or composition, e.g., a polymer-agent conjugate, particle or composition described herein, to a subject with a proliferative disorder, e.g., cancer, in an amount effective to treat the disorder and in the absence of administration of an H1 antagonist or an H2 antagonist, to thereby treat the proliferative disorder.

In an embodiment, the polymer-agent conjugate comprises an anticancer agent such as an epothilone, coupled, e.g., via a linker, to a polymer described herein. In an embodiment, the polymer-agent conjugate comprises an epothilone molecule, coupled via a linker shown in FIG. 1 to a polymer, e.g., a polymer described herein. In an embodiment, the polymer-agent conjugate is a polymer-epothilone conjugate shown in FIG. 1.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the polymer-ixabepilone conjugate, particle or composition is administered at a dose and/or dosing schedule described herein.

In one embodiment, the polymer-agent conjugate, particle or composition includes a polymer-epothilone D conjugate, particle or composition, e.g., a polymer-epothilone D conjugate, particle or composition described herein, e.g., a polymer-epothilone D conjugate comprising an epothilone D molecule, coupled, e.g., via a linker, to a polymer described herein. In an embodiment, the polymer-agent conjugate comprises an epothilone D molecule, coupled via a linker shown in FIG. 1 to a polymer, e.g., a polymer described herein. In an embodiment, the polymer-agent conjugate is a polymer-epothilone D conjugate shown in FIG. 1. In one embodiment, the subject is selected for treatment with the polymer-epothilone D conjugate, particle or composition at a dose and/or dosing schedule described herein.

In one embodiment, the cancer is a cancer described herein. In one embodiment, the polymer-agent conjugate, particle or composition is administered in combination with one or more additional chemotherapeutic agent, e.g., a chemotherapeutic agent or combination of chemotherapeutic agents described herein.

In one aspect, the disclosure features a method of treating a subject, e.g., a human, with a proliferative disorder, e.g., cancer, comprising:

administering a polymer-agent conjugate, particle or composition, e.g., a polymer-agent conjugate, particle or composition e described herein, to a subject with a proliferative disorder, e.g., cancer, in an amount effective to treat the disorder and in combination with an H1 antagonist or an H2 antagonist, wherein the H1 antagonist is administered at a dose of less than 40 mg, 30 mg, 20 mg, 15 mg, 10 mg, 5 mg and/or the H2 antagonist is administered at a dose of less than 140 mg, 130 mg, 120 mg, 100 mg, 90 mg, 80 mg, 70 mg, 60 mg, 50 mg to thereby treat the proliferative disorder.

In an embodiment, the polymer-agent conjugate comprises an anticancer agent such as an epothilone, coupled, e.g., via a linker, to a polymer described herein. In an embodiment, the polymer-agent conjugate comprises an epothilone molecule, coupled via a linker shown in FIG. 1 to a polymer, e.g., a polymer described herein. In an embodiment, the polymer-agent conjugate is a polymer-epothilone conjugate shown in FIG. 1.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the subject is selected for treatment with the polymer-ixabepilone conjugate, particle or composition at a dose and/or dosing schedule described herein.

In one embodiment, the polymer-agent conjugate, particle or composition includes a polymer-epothilone D conjugate, particle or composition, e.g., a polymer-epothilone D conjugate, particle or composition described herein, e.g., a polymer-epothilone D conjugate comprising an epothilone D molecule, coupled, e.g., via a linker, to a polymer described herein. In an embodiment, the polymer-agent conjugate comprises an epothilone D molecule, coupled via a linker shown in FIG. 1 to a polymer, e.g., a polymer described herein. In an embodiment, the polymer-agent conjugate is a polymer-epothilone D conjugate shown in FIG. 1. In one embodiment, the subject is selected for treatment with the polymer-epothilone D conjugate, particle or composition at a dose and/or dosing schedule described herein.

In one embodiment, the cancer is a cancer described herein. In one embodiment, the polymer-agent conjugate, particle or composition is administered in combination with one or more additional chemotherapeutic agent, e.g., a chemotherapeutic agent or combination of chemotherapeutic agents described herein.

In one aspect, the disclosure features a method of selecting a subject, e.g., a human, with a proliferative disorder, e.g., cancer, for treatment with a polymer-agent conjugate, particle or composition, e.g., a polymer-agent conjugate, particle or composition described herein, comprising:

determining alanine aminotransferase (ALT), aspartate aminotransferase (AST) and/or bilirubin levels in a subject having a proliferative disorder; and

selecting a subject having ALT and/or AST levels greater than 2.5 times the upper limit of normal (ULN) and/or bilirubin levels greater than 1 times the ULN for treatment with polymer-agent conjugate, particle or composition, e.g., polymer-agent conjugate, particle or composition described herein, and capecitabine.

In an embodiment, the polymer-agent conjugate comprises an anticancer agent such as an epothilone, coupled, e.g., via a linker, to a polymer described herein. In an embodiment, the polymer-agent conjugate comprises an epothilone molecule, coupled via a linker shown in FIG. 1 to a polymer, e.g., a polymer described herein. In an embodiment, the polymer-agent conjugate is a polymer-epothilone conjugate shown in FIG. 1.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the subject is selected for treatment with the polymer-ixabepilone conjugate, particle or composition at a dose and/or dosing schedule described herein.

In one embodiment, the polymer-agent conjugate, particle or composition includes a polymer-epothilone B conjugate, particle or composition, e.g., a polymer-epothilone B conjugate, particle or composition described herein, e.g., a polymer-epothilone B conjugate comprising an epothilone B molecule, coupled, e.g., via a linker, to a polymer described herein. In an embodiment, the polymer-agent conjugate comprises an epothilone B molecule, coupled via a linker shown in FIG. 1 to a polymer, e.g., a polymer described herein. In an embodiment, the polymer-agent conjugate is a polymer-epothilone B conjugate shown in FIG. 1. In one embodiment, the subject is selected for treatment with the polymer-epothilone B conjugate, particle or composition at a dose and/or dosing schedule described herein.

In one embodiment, the polymer-agent conjugate, particle or composition includes a polymer-epothilone D conjugate, particle or composition, e.g., a polymer-epothilone D conjugate, particle or composition described herein, e.g., a polymer-epothilone D conjugate comprising an epothilone D molecule, coupled, e.g., via a linker, to a polymer described herein. In an embodiment, the polymer-agent conjugate comprises an epothilone D molecule, coupled via a linker shown in FIG. 1 to a polymer, e.g., a polymer described herein. In an embodiment, the polymer-agent conjugate is a polymer-epothilone D conjugate shown in FIG. 1. In one embodiment, the subject is selected for treatment with the polymer-epothilone D conjugate, particle or composition at a dose and/or dosing schedule described herein.

In one embodiment, the polymer-agent conjugate, particle or composition includes a polymer-BMS310705 conjugate, particle or composition, e.g., a polymer-BMS310705 conjugate, particle or composition described herein, e.g., a polymer-BMS310705 conjugate comprising a BMS310705 molecule, coupled, e.g., via a linker, to a polymer described herein. In an embodiment, the polymer-agent conjugate comprises a BMS310705 molecule, coupled via a linker shown in FIG. 1 to a polymer, e.g., a polymer described herein. In an embodiment, the polymer-agent conjugate is a polymer-BMS310705 conjugate shown in FIG. 1. In one embodiment, the subject is selected for treatment with the polymer-BMS310705 conjugate, particle or composition at a dose and/or dosing schedule described herein.

In one embodiment, the polymer-agent conjugate, particle or composition includes a polymer-dehydelone conjugate, particle or composition, e.g., a polymer-dehydelone conjugate, particle or composition described herein, e.g., a polymer-dehydelone conjugate comprising a dehydelone molecule, coupled, e.g., via a linker, to a polymer described herein. In an embodiment, the polymer-agent conjugate comprises a dehydelone molecule, coupled via a linker shown in FIG. 1 to a polymer, e.g., a polymer described herein. In an embodiment, the polymer-agent conjugate is a polymer-dehydelone conjugate shown in FIG. 1. In one embodiment, the subject is selected for treatment with the polymer-dehydelone conjugate, particle or composition at a dose and/or dosing schedule described herein.

In one embodiment, the polymer-agent conjugate, particle or composition includes a polymer-ZK-EPO conjugate, particle or composition, e.g., a polymer-ZK-EPO conjugate, particle or composition described herein, e.g., a polymer-ZK-EPO conjugate comprising a ZK-EPO molecule, coupled, e.g., via a linker, to a polymer described herein. In an embodiment, the polymer-agent conjugate comprises a ZK-EPO molecule, coupled via a linker shown in FIG. 1 to a polymer, e.g., a polymer described herein. In an embodiment, the polymer-agent conjugate is a polymer-ZK-EPO conjugate shown in FIG. 1. In one embodiment, the subject is selected for treatment with the polymer-ZK-EPO conjugate, particle or composition at a dose and/or dosing schedule described herein.

In one embodiment, the cancer is a cancer described herein. In one embodiment, the subject is selected for treatment with the polymer-agent conjugate, particle or composition in combination with one or more additional chemotherapeutic agent, e.g., a chemotherapeutic agent or combination of chemotherapeutic agents described herein.

In one aspect, the disclosure features a method of treating a subject, e.g., a human, having a proliferative disorder, e.g., cancer, comprising:

selecting a subject with a proliferative disorder who has alanine aminotransferase (ALT) and/or aspartate aminotransferase (AST) levels greater than 2.5 times the upper limit of normal (ULN) and/or bilirubin levels greater than 1 time the ULN; and

administering a polymer-agent conjugate, particle or composition, e.g., a polymer-agent conjugate, particle or composition described herein, to the subject in an amount effective to treat the disorder, to thereby treat the proliferative disorder.

In an embodiment, the polymer-agent conjugate comprises an anticancer agent such as an epothilone, coupled, e.g., via a linker, to a polymer described herein. In an embodiment, the polymer-agent conjugate comprises an epothilone molecule, coupled via a linker shown in FIG. 1 to a polymer, e.g., a polymer described herein. In an embodiment, the polymer-agent conjugate is a polymer-epothilone conjugate shown in FIG. 1.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the polymer-ixabepilone conjugate, particle or composition is administered at a dose and/or dosing schedule described herein.

In one embodiment, the polymer-agent conjugate, particle or composition includes a polymer-epothilone B conjugate, particle or composition, e.g., a polymer-epothilone B conjugate, particle or composition described herein, e.g., a polymer-epothilone B conjugate comprising an epothilone B molecule, coupled, e.g., via a linker, to a polymer described herein. In an embodiment, the polymer-agent conjugate comprises an epothilone B molecule, coupled via a linker shown in FIG. 1 to a polymer, e.g., a polymer described herein. In an embodiment, the polymer-agent conjugate is a polymer-epothilone B conjugate shown in FIG. 1. In one embodiment, the polymer-epothilone B conjugate, particle or composition is administered at a dose and/or dosing schedule described herein.

In one embodiment, the polymer-agent conjugate, particle or composition includes a polymer-epothilone D conjugate, particle or composition, e.g., a polymer-epothilone D conjugate, particle or composition described herein, e.g., a polymer-epothilone D conjugate comprising an epothilone D molecule, coupled, e.g., via a linker, to a polymer described herein. In an embodiment, the polymer-agent conjugate comprises an epothilone D molecule, coupled via a linker shown in FIG. 1 to a polymer, e.g., a polymer described herein. In an embodiment, the polymer-agent conjugate is a polymer-epothilone D conjugate shown in FIG. 1. In one embodiment, the polymer-epothilone D conjugate, particle or composition is administered at a dose and/or dosing schedule described herein.

In one embodiment, the polymer-agent conjugate, particle or composition includes a polymer-BMS310705 conjugate, particle or composition, e.g., a polymer-BMS310705 conjugate, particle or composition described herein, e.g., a polymer-BMS310705 conjugate comprising a BMS310705 molecule, coupled, e.g., via a linker, to a polymer described herein. In an embodiment, the polymer-agent conjugate comprises a BMS310705 molecule, coupled via a linker shown in FIG. 1 to a polymer, e.g., a polymer described herein. In an embodiment, the polymer-agent conjugate is a polymer-BMS310705 conjugate shown in FIG. 1. In one embodiment, the polymer-BMS310705 conjugate, particle or composition is administered at a dose and/or dosing schedule described herein.

In one embodiment, the polymer-agent conjugate, particle or composition includes a polymer-dehydelone conjugate, particle or composition, e.g., a polymer-dehydelone conjugate, particle or composition described herein, e.g., a polymer-dehydelone conjugate comprising a dehydelone molecule, coupled, e.g., via a linker, to a polymer described herein. In an embodiment, the polymer-agent conjugate comprises a dehydelone molecule, coupled via a linker shown in FIG. 1 to a polymer, e.g., a polymer described herein. In an embodiment, the polymer-agent conjugate is a polymer-dehydelone conjugate shown in FIG. 1. In one embodiment, the polymer-dehydelone conjugate, particle or composition is administered at a dose and/or dosing schedule described herein.

In one embodiment, the polymer-agent conjugate, particle or composition includes a polymer-ZK-EPO conjugate, particle or composition, e.g., a polymer-ZK-EPO conjugate, particle or composition described herein, e.g., a polymer-ZK-EPO conjugate comprising a ZK-EPO molecule, coupled, e.g., via a linker, to a polymer described herein. In an embodiment, the polymer-agent conjugate comprises a ZK-EPO molecule, coupled via a linker shown in FIG. 1 to a polymer, e.g., a polymer described herein. In an embodiment, the polymer-agent conjugate is a polymer-ZK-EPO conjugate shown in FIG. 1. In one embodiment, the polymer-ZK-EPO conjugate, particle or composition is administered at a dose and/or dosing schedule described herein.

In one embodiment, the cancer is a cancer described herein. In one embodiment, the polymer-agent conjugate, particle or composition is administered in combination with one or more additional chemotherapeutic agent, e.g., a chemotherapeutic agent or combination of chemotherapeutic agents described herein.

In another aspect, the disclosure features a method of selecting a subject, e.g., a human, with a proliferative disorder, e.g., cancer, for treatment with a polymer-agent conjugate, particle or composition, e.g., a polymer-agent conjugate, particle or composition described herein, comprising:

determining alanine aminotransferase (ALT), aspartate aminotransferase (AST) and/or bilirubin levels in a subject having a proliferative disorder; and

selecting a subject having ALT and/or AST levels less than or equal to 10 times the upper limit of normal (ULN) and bilirubin levels are less than or equal to 1.5 times the ULN for treatment with polymer-agent conjugate, particle or composition, e.g., polymer-agent conjugate, particle or composition described herein, at a dose of 40 mg/m² or greater.

In an embodiment, the polymer-agent conjugate comprises an anticancer agent such as an epothilone, coupled, e.g., via a linker, to a polymer described herein. In an embodiment, the polymer-agent conjugate comprises an epothilone molecule, coupled via a linker shown in FIG. 1 to a polymer, e.g., a polymer described herein. In an embodiment, the polymer-agent conjugate is a polymer-epothilone conjugate shown in FIG. 1.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the subject is selected for treatment with the polymer-ixabepilone conjugate, particle or composition in combination with one or more additional agent, e.g., one or more chemotherapeutic agent described herein. In one embodiment, the subject is selected for administration of at least two doses of the polymer-ixabepilone conjugate, particle or composition, e.g., at a dose and/or dosing schedule described herein.

In one aspect, the disclosure features a method of treating a subject, e.g., a human, having a proliferative disorder, e.g., cancer, comprising:

selecting a subject with a proliferative disorder, e.g., cancer, who has alanine aminotransferase (ALT) and/or aspartate aminotransferase (AST) levels less than or equal to 10 times the upper limit of normal (ULN) and bilirubin levels are less than or equal to 1.5 times the ULN; and

administering a polymer-agent conjugate, particle or composition, e.g., a polymer-agent conjugate, particle or composition described herein, to the subject at dose of 40 mg/m², to thereby treat the disorder.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the subject is selected for treatment with the polymer-ixabepilone conjugate, particle or composition in combination with one or more additional agent, e.g., one or more chemotherapeutic agent described herein. In one embodiment, the subject is selected for administration of at least two doses of the polymer-ixabepilone conjugate, particle or composition, e.g., at a dose and/or dosing schedule described herein.

In another aspect, the disclosure features a method of selecting a subject, e.g., a human, with a proliferative disorder, e.g., cancer, for treatment with a polymer-agent conjugate, particle or composition, e.g., a polymer-agent conjugate, particle or composition described herein, comprising:

determining alanine aminotransferase (ALT), aspartate aminotransferase (AST) and/or bilirubin levels in a subject having a proliferative disorder; and

selecting a subject having alanine aminotransferase and/or aspartate aminotransferase levels less than or equal to 10 times the upper limit of normal (ULN) and bilirubin levels in the range of greater than 1.5 times the ULN to less than or equal to 3 times the ULN for treatment with a polymer-agent conjugate, particle or composition, e.g., a polymer-agent conjugate, particle or composition described herein, at a dose of 40 mg/m².

In an embodiment, the polymer-agent conjugate comprises an anticancer agent such as an epothilone, coupled, e.g., via a linker, to a polymer described herein. In an embodiment, the polymer-agent conjugate comprises an epothilone molecule, coupled via a linker shown in FIG. 1 to a polymer, e.g., a polymer described herein. In an embodiment, the polymer-agent conjugate is a polymer-epothilone conjugate shown in FIG. 1.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the subject is selected for treatment with the polymer-ixabepilone conjugate, particle or composition in combination with one or more additional agent, e.g., one or more chemotherapeutic agent described herein. In one embodiment, the subject is selected for treatment with at least two doses of the polymer-ixabepilone conjugate, particle or composition, e.g., at a dose and/or dosing schedule described herein.

In one aspect, the disclosure features a method of treating a subject, e.g., a human, having a proliferative disorder, e.g., cancer, comprising:

selecting a subject with a proliferative disorder, e.g., cancer, who has alanine aminotransferase (ALT) and/or aspartate aminotransferase (AST) levels less than or equal to 10 times the upper limit of normal (ULN) and bilirubin levels in the range of greater than 1.5 times the ULN to less than or equal to 3 times the ULN; and

administering a polymer-agent conjugate, particle or composition, e.g., a polymer-agent conjugate, particle or composition described herein, to the subject at dose of 40 mg/m², to thereby treat the disorder.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the polymer-ixabepilone conjugate, particle or composition is administered in combination with one or more additional agent, e.g., one or more chemotherapeutic agent described herein. In one embodiment, the subject is administered at least an additional dose of the polymer-ixabepilone conjugate, particle or composition, e.g., at a dose and/or dosing schedule described herein.

In one aspect, the disclosure features a method of selecting a subject, e.g., a human, with a proliferative disorder, e.g., cancer, for treatment with a polymer-agent conjugate, particle or composition, e.g., a polymer-agent conjugate, particle or composition described herein, comprising:

determining if a subject having a proliferative disorder is currently being administered (e.g., the subject has been administered a CYP3A4 inhibitor the same day as chemotherapy treatment or within 1, 2, 3, 4, 5, 6, or 7 days before chemotherapy treatment) or will be administered (e.g., will be administered on the same day as the chemotherapy treatment or within 1, 2, 3, 4, 5, 6, or 7 days after chemotherapy treatment) a CYP3A4 inhibitor (e.g., ketoconazole, itraconazole, clarithromycin, atazanavir, nefazodone, saquinavir, telithromycin, ritonavir, amprenavir, indinavir, nelfinavir, delavirdine or voriconazole); and

selecting a subject with a proliferative disorder, e.g., cancer, that is currently being administered or will be administered a CYP3A4 inhibitor for treatment with a polymer-agent conjugate, particle or composition, e.g., a polymer-agent conjugate, particle or composition described herein, at a dose of 40 mg/m².

In an embodiment, the polymer-agent conjugate comprises an anticancer agent such as an epothilone, coupled, e.g., via a linker, to a polymer described herein. In an embodiment, the polymer-agent conjugate comprises an epothilone molecule, coupled via a linker shown in FIG. 1 to a polymer, e.g., a polymer described herein. In an embodiment, the polymer-agent conjugate is a polymer-epothilone conjugate shown in FIG. 1.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the subject is selected for treatment with the polymer-ixabepilone conjugate, particle or composition in combination with one or more additional agent, e.g., one or more chemotherapeutic agent described herein. In one embodiment, the subject is selected for administration of at least two doses of the polymer-ixabepilone conjugate, particle or composition, e.g., at a dose and/or dosing schedule described herein.

In another aspect, the disclosure features a method of treating a subject, e.g., a human, having a proliferative disorder, e.g., cancer, comprising:

selecting a subject with a proliferative disorder, e.g., cancer, who is currently being administered or will be, administered a CYP3A4 inhibitor; and

administering a polymer-agent conjugate, particle or composition, e.g., a polymer-agent conjugate, particle or composition described herein, to the subject at dose of 40 mg/m², to thereby treat the disorder.

In an embodiment, the polymer-agent conjugate comprises an anticancer agent such as an epothilone, coupled, e.g., via a linker, to a polymer described herein. In an embodiment, the polymer-agent conjugate comprises an epothilone molecule, coupled via a linker shown in FIG. 1 to a polymer, e.g., a polymer described herein. In an embodiment, the polymer-agent conjugate is a polymer-epothilone conjugate shown in FIG. 1.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the polymer-ixabepilone conjugate, particle or composition is administered in combination with one or more additional agent, e.g., one or more chemotherapeutic agent described herein. In one embodiment, the subject is administered at least an additional dose of the polymer-ixabepilone conjugate, particle or composition, e.g., at a dose and/or dosing schedule described herein.

In one aspect, the disclosure features a method of selecting a subject, e.g., a human, with a proliferative disorder, e.g., cancer, for treatment with a polymer-agent conjugate, particle or composition, e.g., a polymer-agent conjugate, particle or composition described herein, comprising:

determining if a subject having a proliferative disorder is currently being administered or will be administered an anti-depressant; and

selecting a subject who is currently being administered or will be administered an anti-depressant, e.g., St. John's wort, for treatment with a polymer-agent conjugate, particle or composition, e.g., a polymer-agent conjugate, particle or composition described herein.

In one embodiment, the anti-depressant is one or more of a monoamine oxidase inhibitor (MAOI), a tricyclic antidepressant (TCA), a tetracyclic antidepressant (TeCA), a selective serotonin reuptake inhibitor (SSRI), and a serotonin-norepinephrine reuptake inhibitor (SNRI). In one embodiment, the anti-depressant is St. John's wort.

In an embodiment, the polymer-agent conjugate comprises an anticancer agent such as an epothilone, coupled, e.g., via a linker, to a polymer described herein. In an embodiment, the polymer-agent conjugate comprises an epothilone molecule, coupled via a linker shown in FIG. 1 to a polymer, e.g., a polymer described herein. In an embodiment, the polymer-agent conjugate is a polymer-epothilone conjugate shown in FIG. 1.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the subject is selected for treatment with the polymer-ixabepilone conjugate, particle or composition in combination with one or more additional agent, e.g., one or more chemotherapeutic agent described herein. In one embodiment, the subject is selected for administration of at least two doses of the polymer-ixabepilone conjugate, particle or composition, e.g., at a dose and/or dosing schedule described herein.

In another aspect, the disclosure features a method of treating a subject, e.g., a human, having a proliferative disorder, e.g., cancer, comprising:

selecting a subject with a proliferative disorder, e.g., cancer, that is currently being administered or will be administered an antidepressant; and

administering a polymer-agent conjugate, particle or composition, e.g., a polymer-agent conjugate, particle or composition described herein, to the subject in an amount effective to treat the disorder, to thereby treat the disorder.

In one embodiment, the anti-depressant is one or more of a monoamine oxidase inhibitor (MAOI), a tricyclic antidepressant (TCA), a tetracyclic antidepressant (TeCA), a selective serotonin reuptake inhibitor (SSRI), and a serotonin-norepinephrine reuptake inhibitor (SNRI). In one embodiment, the anti-depressant is St. John's wort.

In an embodiment, the polymer-agent conjugate comprises an anticancer agent such as an epothilone, coupled, e.g., via a linker, to a polymer described herein. In an embodiment, the polymer-agent conjugate comprises an epothilone molecule, coupled via a linker shown in FIG. 1 to a polymer, e.g., a polymer described herein. In an embodiment, the polymer-agent conjugate is a polymer-epothilone conjugate shown in FIG. 1.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the polymer-ixabepilone conjugate, particle or composition is administered in combination with one or more additional agent, e.g., one or more chemotherapeutic agent described herein. In one embodiment, the subject is administered at least an additional dose of the polymer-ixabepilone conjugate, particle or composition, e.g., at a dose and/or dosing schedule described herein.

In one aspect, the disclosure features a method of selecting a subject, e.g., a human, with a proliferative disorder, e.g., cancer, for treatment with a polymer-agent conjugate, particle or composition, e.g., polymer-agent conjugate, particle or composition described herein, comprising:

determining if a subject having a proliferative disorder is 65 or older; and

selecting a subject who is 65 or older for treatment with a polymer-agent conjugate, particle or composition, e.g., a polymer-agent conjugate, particle or composition described herein.

In an embodiment, the polymer-agent conjugate comprises an anticancer agent such as an epothilone, coupled, e.g., via a linker, to a polymer described herein. In an embodiment, the polymer-agent conjugate comprises an epothilone molecule, coupled via a linker shown in FIG. 1 to a polymer, e.g., a polymer described herein. In an embodiment, the polymer-agent conjugate is a polymer-epothilone conjugate shown in FIG. 1.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the subject is selected for treatment with the polymer-ixabepilone conjugate, particle or composition in combination with one or more additional agent, e.g., one or more chemotherapeutic agent described herein. In one embodiment, the subject is selected for administration of at least an additional dose of the polymer-ixabepilone conjugate, particle or composition, e.g., at a dose and/or dosing schedule described herein.

In one embodiment, the polymer-agent conjugate, particle or composition includes a polymer-epothilone B conjugate, particle or composition, e.g., a polymer-epothilone B conjugate, particle or composition described herein, e.g., a polymer-epothilone B conjugate comprising an epothilone B molecule, coupled, e.g., via a linker, to a polymer described herein. In an embodiment, the polymer-agent conjugate comprises an epothilone B molecule, coupled via a linker shown in FIG. 1 to a polymer, e.g., a polymer described herein. In an embodiment, the polymer-agent conjugate is a polymer-epothilone B conjugate shown in FIG. 1. In one embodiment, the subject is selected for treatment with the polymer-epothilone B conjugate, particle or composition in combination with one or more additional agent, e.g., one or more chemotherapeutic agent described herein. In one embodiment, the subject is selected for administration of at least an additional dose of the polymer-epothilone B conjugate, particle or composition, e.g., at a dose and/or dosing schedule described herein.

In one embodiment, the polymer-agent conjugate, particle or composition includes a polymer-epothilone D conjugate, particle or composition, e.g., a polymer-epothilone D conjugate, particle or composition described herein, e.g., a polymer-epothilone D conjugate comprising an epothilone D molecule, coupled, e.g., via a linker, to a polymer described herein. In an embodiment, the polymer-agent conjugate comprises an epothilone D molecule, coupled via a linker shown in FIG. 1 to a polymer, e.g., a polymer described herein. In an embodiment, the polymer-agent conjugate is a polymer-epothilone D conjugate shown in FIG. 1. In one embodiment, the subject is selected for treatment with the polymer-epothilone D conjugate, particle or composition in combination with one or more additional agent, e.g., one or more chemotherapeutic agent described herein. In one embodiment, the subject is selected for administration of at least an additional dose of the polymer-epothilone D conjugate, particle or composition, e.g., at a dose and/or dosing schedule described herein.

In one embodiment, the polymer-agent conjugate, particle or composition includes a polymer-BMS310705 conjugate, particle or composition, e.g., a polymer-BMS310705 conjugate, particle or composition described herein, e.g., a polymer-BMS310705 conjugate comprising a BMS310705 molecule, coupled, e.g., via a linker, to a polymer described herein. In an embodiment, the polymer-agent conjugate comprises a BMS310705 molecule, coupled via a linker shown in FIG. 1 to a polymer, e.g., a polymer described herein. In an embodiment, the polymer-agent conjugate is a polymer-BMS310705 conjugate shown in FIG. 1. In one embodiment, the subject is selected for treatment with the polymer-BMS310705 conjugate, particle or composition in combination with one or more additional agent, e.g., one or more chemotherapeutic agent described herein. In one embodiment, the subject is selected for administration of at least an additional dose of the polymer-BMS310705 conjugate, particle or composition, e.g., at a dose and/or dosing schedule described herein.

In one embodiment, the polymer-agent conjugate, particle or composition includes a polymer-dehydelone conjugate, particle or composition, e.g., a polymer-dehydelone conjugate, particle or composition described herein, e.g., a polymer-dehydelone conjugate comprising a dehydelone molecule, coupled, e.g., via a linker, to a polymer described herein. In an embodiment, the polymer-agent conjugate comprises a dehydelone molecule, coupled via a linker shown in FIG. 1 to a polymer, e.g., a polymer described herein. In an embodiment, the polymer-agent conjugate is a polymer-dehydelone conjugate shown in FIG. 1. In one embodiment, the subject is selected for treatment with the polymer-dehydelone conjugate, particle or composition in combination with one or more additional agent, e.g., one or more chemotherapeutic agent described herein. In one embodiment, the subject is selected for administration of at least an additional dose of the polymer-dehydelone conjugate, particle or composition, e.g., at a dose and/or dosing schedule described herein.

In one embodiment, the polymer-agent conjugate, particle or composition includes a polymer-ZK-EPO conjugate, particle or composition, e.g., a polymer-ZK-EPO conjugate, particle or composition described herein, e.g., a polymer-ZK-EPO conjugate comprising a ZK-EPO molecule, coupled, e.g., via a linker, to a polymer described herein. In an embodiment, the polymer-agent conjugate comprises a ZK-EPO molecule, coupled via a linker shown in FIG. 1 to a polymer, e.g., a polymer described herein. In an embodiment, the polymer-agent conjugate is a polymer-ZK-EPO conjugate shown in FIG. 1. In one embodiment, the subject is selected for treatment with the polymer-ZK-EPO conjugate, particle or composition in combination with one or more additional agent, e.g., one or more chemotherapeutic agent described herein. In one embodiment, the subject is selected for administration of at least an additional dose of the polymer-ZK-EPO conjugate, particle or composition, e.g., at a dose and/or dosing schedule described herein.

In one aspect, the disclosure features a method of treating a subject, e.g., a human, having a proliferative disorder, e.g., cancer, comprising:

selecting a subject with a proliferative disorder, e.g., a cancer, who is 65 or older; and

administering a polymer-agent conjugate, particle or composition, e.g., a polymer-agent conjugate, particle or composition described herein, and capecitabine to the subject in an amount effective to treat the disorder, to thereby treat the disorder.

In an embodiment, the polymer-agent conjugate comprises an anticancer agent such as an epothilone, coupled, e.g., via a linker, to a polymer described herein. In an embodiment, the polymer-agent conjugate comprises an epothilone molecule, coupled via a linker shown in FIG. 1 to a polymer, e.g., a polymer described herein. In an embodiment, the polymer-agent conjugate is a polymer-epothilone conjugate shown in FIG. 1.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the polymer-ixabepilone conjugate, particle or composition is administered in combination with one or more additional agent, e.g., one or more chemotherapeutic agent described herein. In one embodiment, at least an additional dose of the polymer-ixabepilone conjugate, particle or composition is administered, e.g., at a dose and/or dosing schedule described herein.

In one embodiment, the polymer-agent conjugate, particle or composition includes a polymer-epothilone B conjugate, particle or composition, e.g., a polymer-epothilone B conjugate, particle or composition described herein, e.g., a polymer-epothilone B conjugate comprising an epothilone B molecule, coupled, e.g., via a linker, to a polymer described herein. In an embodiment, the polymer-agent conjugate comprises an epothilone B molecule, coupled via a linker shown in FIG. 1 to a polymer, e.g., a polymer described herein. In an embodiment, the polymer-agent conjugate is a polymer-epothilone B conjugate shown in FIG. 1. In one embodiment, the polymer-epothilone B conjugate, particle or composition is administered in combination with one or more additional agent, e.g., one or more chemotherapeutic agent described herein. In one embodiment, at least an additional dose of the polymer-epothilone B conjugate, particle or composition is administered, e.g., at a dose and/or dosing schedule described herein.

In one embodiment, the polymer-agent conjugate, particle or composition includes a polymer-epothilone D conjugate, particle or composition, e.g., a polymer-epothilone D conjugate, particle or composition described herein, e.g., a polymer-epothilone D conjugate comprising an epothilone D molecule, coupled, e.g., via a linker, to a polymer described herein. In an embodiment, the polymer-agent conjugate comprises an epothilone D molecule, coupled via a linker shown in FIG. 1 to a polymer, e.g., a polymer described herein. In an embodiment, the polymer-agent conjugate is a polymer-epothilone D conjugate shown in FIG. 1. In one embodiment, the polymer-epothilone D conjugate, particle or composition is administered in combination with one or more additional agent, e.g., one or more chemotherapeutic agent described herein. In one embodiment, at least an additional dose of the polymer-epothilone D conjugate, particle or composition is administered, e.g., at a dose and/or dosing schedule described herein.

In one embodiment, the polymer-agent conjugate, particle or composition includes a polymer-BMS310705 conjugate, particle or composition, e.g., a polymer-BMS310705 conjugate, particle or composition described herein, e.g., a polymer-BMS310705 conjugate comprising a BMS310705 molecule, coupled, e.g., via a linker, to a polymer described herein. In an embodiment, the polymer-agent conjugate comprises a BMS310705 molecule, coupled via a linker shown in FIG. 1 to a polymer, e.g., a polymer described herein. In an embodiment, the polymer-agent conjugate is a polymer-BMS310705 conjugate shown in FIG. 1. In one embodiment, the polymer-BMS310705 conjugate, particle or composition is administered in combination with one or more additional agent, e.g., one or more chemotherapeutic agent described herein. In one embodiment, at least an additional dose of the polymer-BMS310705 conjugate, particle or composition is administered, e.g., at a dose and/or dosing schedule described herein.

In one embodiment, the polymer-agent conjugate, particle or composition includes a polymer-dehydelone conjugate, particle or composition, e.g., a polymer-dehydelone conjugate, particle or composition described herein, e.g., a polymer-dehydelone conjugate comprising a dehydelone molecule, coupled, e.g., via a linker, to a polymer described herein. In an embodiment, the polymer-agent conjugate comprises a dehydelone molecule, coupled via a linker shown in FIG. 1 to a polymer, e.g., a polymer described herein. In an embodiment, the polymer-agent conjugate is a polymer-dehydelone conjugate shown in FIG. 1. In one embodiment, the polymer-dehydelone conjugate, particle or composition is administered in combination with one or more additional agent, e.g., one or more chemotherapeutic agent described herein. In one embodiment, at least an additional dose of the polymer-dehydelone conjugate, particle or composition is administered, e.g., at a dose and/or dosing schedule described herein.

In one embodiment, the polymer-agent conjugate, particle or composition is a polymer-ZK-EPO conjugate, particle or composition, e.g., a polymer-ZK-EPO conjugate, particle or composition described herein, e.g., a polymer-ZK-EPO conjugate comprising a ZK-EPO molecule, coupled, e.g., via a linker, to a polymer described herein. In an embodiment, the polymer-agent conjugate comprises a ZK-EPO molecule, coupled via a linker shown in FIG. 1 to a polymer, e.g., a polymer described herein. In an embodiment, the polymer-agent conjugate is a polymer-ZK-EPO conjugate shown in FIG. 1. In one embodiment, the polymer-ZK-EPO conjugate, particle or composition is administered in combination with one or more additional agent, e.g., one or more chemotherapeutic agent described herein. In one embodiment, at least an additional dose of the polymer-ZK-EPO conjugate, particle or composition includes administered, e.g., at a dose and/or dosing schedule described herein.

In another aspect, the disclosure features a method of selecting a subject, e.g., a human, with a proliferative disorder, e.g., cancer, for treatment with a polymer-agent conjugate, particle or composition, e.g., a polymer-agent conjugate, particle or composition described herein, comprising:

determining if a subject with a proliferative disorder, e.g., a cancer, is at risk for or has or previously had a cardiac adverse reaction; and

selecting a subject who is at risk for or has or previously had a cardiac adverse reaction for treatment with a polymer-agent conjugate, particle or composition, e.g., a polymer-agent conjugate, particle or composition described herein.

In an embodiment, the polymer-agent conjugate comprises an anticancer agent such as an epothilone, coupled, e.g., via a linker, to a polymer described herein. In an embodiment, the polymer-agent conjugate comprises an epothilone molecule, coupled via a linker shown in FIG. 1 to a polymer, e.g., a polymer described herein. In an embodiment, the polymer-agent conjugate is a polymer-epothilone conjugate shown in FIG. 1.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the subject is selected for treatment with the polymer-ixabepilone conjugate, particle or composition in combination with one or more additional agent, e.g., one or more chemotherapeutic agent described herein. In one embodiment, the subject is selected for administration of at least an additional dose of the polymer-ixabepilone conjugate, particle or composition, e.g., at a dose and/or dosing schedule described herein.

In one embodiment, the polymer-agent conjugate, particle or composition includes a polymer-epothilone B conjugate, particle or composition, e.g., a polymer-epothilone B conjugate, particle or composition described herein, e.g., a polymer-epothilone B conjugate comprising an epothilone B molecule, coupled, e.g., via a linker, to a polymer described herein. In an embodiment, the polymer-agent conjugate comprises an epothilone B molecule, coupled via a linker shown in FIG. 1 to a polymer, e.g., a polymer described herein. In an embodiment, the polymer-agent conjugate is a polymer-epothilone B conjugate shown in FIG. 1. In one embodiment, the subject is selected for treatment with the polymer-epothilone B conjugate, particle or composition in combination with one or more additional agent, e.g., one or more chemotherapeutic agent described herein. In one embodiment, the subject is selected for administration of at least an additional dose of the polymer-epothilone B conjugate, particle or composition, e.g., at a dose and/or dosing schedule described herein.

In one embodiment, the polymer-agent conjugate, particle or composition includes a polymer-epothilone D conjugate, particle or composition, e.g., a polymer-epothilone D conjugate, particle or composition described herein, e.g., a polymer-epothilone D conjugate comprising an epothilone D molecule, coupled, e.g., via a linker, to a polymer described herein. In an embodiment, the polymer-agent conjugate comprises an epothilone D molecule, coupled via a linker shown in FIG. 1 to a polymer, e.g., a polymer described herein. In an embodiment, the polymer-agent conjugate is a polymer-epothilone D conjugate shown in FIG. 1. In one embodiment, the subject is selected for treatment with the polymer-epothilone D conjugate, particle or composition in combination with one or more additional agent, e.g., one or more chemotherapeutic agent described herein. In one embodiment, the subject is selected for administration of at least an additional dose of the polymer-epothilone D conjugate, particle or composition, e.g., at a dose and/or dosing schedule described herein.

In one embodiment, the polymer-agent conjugate, particle or composition includes a polymer-BMS310705 conjugate, particle or composition, e.g., a polymer-BMS310705 conjugate, particle or composition described herein, e.g., a polymer-BMS310705 conjugate comprising a BMS310705 molecule, coupled, e.g., via a linker, to a polymer described herein. In an embodiment, the polymer-agent conjugate comprises a BMS310705 molecule, coupled via a linker shown in FIG. 1 to a polymer, e.g., a polymer described herein. In an embodiment, the polymer-agent conjugate is a polymer-BMS310705 conjugate shown in FIG. 1. In one embodiment, the subject is selected for treatment with the polymer-BMS310705 conjugate, particle or composition in combination with one or more additional agent, e.g., one or more chemotherapeutic agent described herein. In one embodiment, the subject is selected for administration of at least an additional dose of the polymer-BMS310705 conjugate, particle or composition, e.g., at a dose and/or dosing schedule described herein.

In one embodiment, the polymer-agent conjugate, particle or composition includes a polymer-dehydelone conjugate, particle or composition, e.g., a polymer-dehydelone conjugate, particle or composition described herein, e.g., a polymer-dehydelone conjugate comprising a dehydelone molecule, coupled, e.g., via a linker, to a polymer described herein. In an embodiment, the polymer-agent conjugate comprises a dehydelone molecule, coupled via a linker shown in FIG. 1 to a polymer, e.g., a polymer described herein. In an embodiment, the polymer-agent conjugate is a polymer-dehydelone conjugate shown in FIG. 1. In one embodiment, the subject is selected for treatment with the polymer-dehydelone conjugate, particle or composition in combination with one or more additional agent, e.g., one or more chemotherapeutic agent described herein. In one embodiment, the subject is selected for administration of at least an additional dose of the polymer-dehydelone conjugate, particle or composition, e.g., at a dose and/or dosing schedule described herein.

In one embodiment, the polymer-agent conjugate, particle or composition includes a polymer-ZK-EPO conjugate, particle or composition, e.g., a polymer-ZK-EPO conjugate, particle or composition described herein, e.g., a polymer-ZK-EPO conjugate comprising a ZK-EPO molecule, coupled, e.g., via a linker, to a polymer described herein. In an embodiment, the polymer-agent conjugate comprises a ZK-EPO molecule, coupled via a linker shown in FIG. 1 to a polymer, e.g., a polymer described herein. In an embodiment, the polymer-agent conjugate is a polymer-ZK-EPO conjugate shown in FIG. 1. In one embodiment, the subject is selected for treatment with the polymer-ZK-EPO conjugate, particle or composition in combination with one or more additional agent, e.g., one or more chemotherapeutic agent described herein. In one embodiment, the subject is selected for administration of at least an additional dose of the polymer-ZK-EPO conjugate, particle or composition, e.g., at a dose and/or dosing schedule described herein.

In one embodiment, a cardiac adverse reaction includes, e.g., myocardial ischemia, ventricular dysfunction, impaired cardiac function, myocardial infarction, supraventricular arrhythmia, left ventricular dysfunction, angia pectoris, atrial flutter, congestive heart failure (e.g., New York Heart Association class III or class IV heart failure), cardiac insufficiency, congenital long QT syndrome and cardiomyopathy.

In one aspect, the disclosure features a method of treating a subject, e.g., a human, having a proliferative disorder, e.g., cancer, comprising:

selecting a subject with a proliferative disorder, e.g., cancer, who is at risk for or has or previously had a cardiac adverse reaction; and

administering a polymer-agent conjugate, particle or composition, e.g., a polymer-agent conjugate, particle or composition described herein, to the subject in an amount effective to treat the disorder, to thereby treat the disorder.

In an embodiment, the polymer-agent conjugate comprises an anticancer agent such as an epothilone, coupled, e.g., via a linker, to a polymer described herein. In an embodiment, the polymer-agent conjugate comprises an epothilone molecule, coupled via a linker shown in FIG. 1 to a polymer, e.g., a polymer described herein. In an embodiment, the polymer-agent conjugate is a polymer-epothilone conjugate shown in FIG. 1.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the polymer-ixabepilone conjugate, particle or composition is administered in combination with one or more additional agent, e.g., one or more chemotherapeutic agent described herein. In one embodiment, at least an additional dose of the polymer-ixabepilone conjugate, particle or composition is administered, e.g., at a dose and/or dosing schedule described herein.

In one embodiment, the polymer-agent conjugate, particle or composition includes a polymer-epothilone B conjugate, particle or composition, e.g., a polymer-epothilone B conjugate, particle or composition described herein, e.g., a polymer-epothilone B conjugate comprising an epothilone B molecule, coupled, e.g., via a linker, to a polymer described herein. In an embodiment, the polymer-agent conjugate comprises an epothilone B molecule, coupled via a linker shown in FIG. 1 to a polymer, e.g., a polymer described herein. In an embodiment, the polymer-agent conjugate is a polymer-epothilone B conjugate shown in FIG. 1. In one embodiment, the polymer-epothilone B conjugate, particle or composition is administered in combination with one or more additional agent, e.g., one or more chemotherapeutic agent described herein. In one embodiment, at least an additional dose of the polymer-epothilone B conjugate, particle or composition is administered, e.g., at a dose and/or dosing schedule described herein.

In one embodiment, the polymer-agent conjugate, particle or composition includes a polymer-epothilone D conjugate, particle or composition, e.g., a polymer-epothilone D conjugate, particle or composition described herein, e.g., a polymer-epothilone D conjugate comprising an epothilone D molecule, coupled, e.g., via a linker, to a polymer described herein. In an embodiment, the polymer-agent conjugate comprises an epothilone D molecule, coupled via a linker shown in FIG. 1 to a polymer, e.g., a polymer described herein. In an embodiment, the polymer-agent conjugate is a polymer-epothilone D conjugate shown in FIG. 1. In one embodiment, the polymer-epothilone D conjugate, particle or composition is administered in combination with one or more additional agent, e.g., one or more chemotherapeutic agent described herein. In one embodiment, at least an additional dose of the polymer-epothilone D conjugate, particle or composition is administered, e.g., at a dose and/or dosing schedule described herein.

In one embodiment, the polymer-agent conjugate, particle or composition includes a polymer-BMS310705 conjugate, particle or composition, e.g., a polymer-BMS310705 conjugate, particle or composition described herein, e.g., a polymer-BMS310705 conjugate comprising a BMS310705 molecule, coupled, e.g., via a linker, to a polymer described herein. In an embodiment, the polymer-agent conjugate comprises a BMS310705 molecule, coupled via a linker shown in FIG. 1 to a polymer, e.g., a polymer described herein. In an embodiment, the polymer-agent conjugate is a polymer-BMS310705 conjugate shown in FIG. 1. In one embodiment, the polymer-BMS310705 conjugate, particle or composition is administered in combination with one or more additional agent, e.g., one or more chemotherapeutic agent described herein. In one embodiment, at least an additional dose of the polymer-BMS310705 conjugate, particle or composition is administered, e.g., at a dose and/or dosing schedule described herein.

In one embodiment, the polymer-agent conjugate, particle or composition includes a polymer-dehydelone conjugate, particle or composition, e.g., a polymer-dehydelone conjugate, particle or composition described herein, e.g., a polymer-dehydelone conjugate comprising a dehydelone molecule, coupled, e.g., via a linker, to a polymer described herein. In an embodiment, the polymer-agent conjugate comprises a dehydelone molecule, coupled via a linker shown in FIG. 1 to a polymer, e.g., a polymer described herein. In an embodiment, the polymer-agent conjugate is a polymer-dehydelone conjugate shown in FIG. 1. In one embodiment, the polymer-dehydelone conjugate, particle or composition is administered in combination with one or more additional agent, e.g., one or more chemotherapeutic agent described herein. In one embodiment, at least an additional dose of the polymer-dehydelone conjugate, particle or composition is administered, e.g., at a dose and/or dosing schedule described herein.

In one embodiment, the polymer-agent conjugate, particle or composition is a polymer-ZK-EPO conjugate, particle or composition, e.g., a polymer-ZK-EPO conjugate, particle or composition described herein, e.g., a polymer-ZK-EPO conjugate comprising a ZK-EPO molecule, coupled, e.g., via a linker, to a polymer described herein. In an embodiment, the polymer-agent conjugate comprises a ZK-EPO molecule, coupled via a linker shown in FIG. 1 to a polymer, e.g., a polymer described herein. In an embodiment, the polymer-agent conjugate is a polymer-ZK-EPO conjugate shown in FIG. 1. In one embodiment, the polymer-ZK-EPO conjugate, particle or composition is administered in combination with one or more additional agent, e.g., one or more chemotherapeutic agent described herein. In one embodiment, at least an additional dose of the polymer-ZK-EPO conjugate, particle or composition is administered, e.g., at a dose and/or dosing schedule described herein.

In one embodiment, a cardiac adverse reaction includes, e.g., myocardial ischemia, ventricular dysfunction, impaired cardiac function, myocardial infarction, supraventricular arrhythmia, left ventricular dysfunction, angia pectoris, artrial flutter, congestive heart failure (e.g., New York Heart Association class III or class IV heart failure), cardiac insufficiency, congenital long QT syndrome and cardiomyopathy.

In one aspect, the disclosure features a method of identifying a subject, e.g., a human, having a proliferative disorder, e.g., cancer, for treatment with a polymer-agent conjugate, particle or composition, e.g., a polymer-agent conjugate, particle or composition described herein, the method comprising:

identifying a subject having a proliferative disorder who has received an epothilone (e.g., ixabepilone, epothilone B, epothilone D, BMS310705, dehydelone or ZK-EPO) and has a platelet count less than a standard; and

identifying the subject as suitable for treatment with a polymer-agent conjugate, particle or composition, e.g., a polymer-agent conjugate, particle or composition described herein.

In one embodiment, the method further comprising administering a polymer-agent conjugate, particle or composition, e.g., a polymer-agent conjugate, particle or composition described herein in an amount effective to treat the disorder.

In an embodiment, the polymer-agent conjugate comprises an anticancer agent such as an epothilone, coupled, e.g., via a linker, to a polymer described herein. In an embodiment, the polymer-agent conjugate comprises an epothilone molecule, coupled via a linker shown in FIG. 1 to a polymer, e.g., a polymer described herein. In an embodiment, the polymer-agent conjugate is a polymer-epothilone conjugate shown in FIG. 1.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the polymer-ixabepilone conjugate, particle or composition is administered in combination with one or more additional agent, e.g., one or more chemotherapeutic agent described herein. In one embodiment, at least an additional dose of the polymer-ixabepilone conjugate, particle or composition is administered, e.g., at a dose and/or dosing schedule described herein.

In one embodiment, the polymer-agent conjugate, particle or composition includes a polymer-epothilone B conjugate, particle or composition, e.g., a polymer-epothilone B conjugate, particle or composition described herein, e.g., a polymer-epothilone B conjugate comprising an epothilone B molecule, coupled, e.g., via a linker, to a polymer described herein. In an embodiment, the polymer-agent conjugate comprises an epothilone B molecule, coupled via a linker shown in FIG. 1 to a polymer, e.g., a polymer described herein. In an embodiment, the polymer-agent conjugate is a polymer-epothilone B conjugate shown in FIG. 1. In one embodiment, the polymer-epothilone B conjugate, particle or composition is administered in combination with one or more additional agent, e.g., one or more chemotherapeutic agent described herein. In one embodiment, at least an additional dose of the polymer-epothilone B conjugate, particle or composition is administered, e.g., at a dose and/or dosing schedule described herein.

In one embodiment, the polymer-agent conjugate, particle or composition includes a polymer-epothilone D conjugate, particle or composition, e.g., a polymer-epothilone D conjugate, particle or composition described herein, e.g., a polymer-epothilone D conjugate comprising an epothilone D molecule, coupled, e.g., via a linker, to a polymer described herein. In an embodiment, the polymer-agent conjugate comprises an epothilone D molecule, coupled via a linker shown in FIG. 1 to a polymer, e.g., a polymer described herein. In an embodiment, the polymer-agent conjugate is a polymer-epothilone D conjugate shown in FIG. 1. In one embodiment, the polymer-epothilone D conjugate, particle or composition is administered in combination with one or more additional agent, e.g., one or more chemotherapeutic agent described herein. In one embodiment, at least an additional dose of the polymer-epothilone D conjugate, particle or composition is administered, e.g., at a dose and/or dosing schedule described herein.

In one embodiment, the polymer-agent conjugate, particle or composition includes a polymer-BMS310705 conjugate, particle or composition, e.g., a polymer-BMS310705 conjugate, particle or composition described herein, e.g., a polymer-BMS310705 conjugate comprising a BMS310705 molecule, coupled, e.g., via a linker, to a polymer described herein. In an embodiment, the polymer-agent conjugate comprises a BMS310705 molecule, coupled via a linker shown in FIG. 1 to a polymer, e.g., a polymer described herein. In an embodiment, the polymer-agent conjugate is a polymer-BMS310705 conjugate shown in FIG. 1. In one embodiment, the polymer-BMS310705 conjugate, particle or composition is administered in combination with one or more additional agent, e.g., one or more chemotherapeutic agent described herein. In one embodiment, at least an additional dose of the polymer-BMS310705 conjugate, particle or composition is administered, e.g., at a dose and/or dosing schedule described herein.

In one embodiment, the polymer-agent conjugate, particle or composition includes a polymer-dehydelone conjugate, particle or composition, e.g., a polymer-dehydelone conjugate, particle or composition described herein, e.g., a polymer-dehydelone conjugate comprising a dehydelone molecule, coupled, e.g., via a linker, to a polymer described herein. In an embodiment, the polymer-agent conjugate comprises a dehydelone molecule, coupled via a linker shown in FIG. 1 to a polymer, e.g., a polymer described herein. In an embodiment, the polymer-agent conjugate is a polymer-dehydelone conjugate shown in FIG. 1. In one embodiment, the polymer-dehydelone conjugate, particle or composition is administered in combination with one or more additional agent, e.g., one or more chemotherapeutic agent described herein. In one embodiment, at least an additional dose of the polymer-dehydelone conjugate, particle or composition is administered, e.g., at a dose and/or dosing schedule described herein.

In one embodiment, the polymer-agent conjugate, particle or composition is a polymer-ZK-EPO conjugate, particle or composition, e.g., a polymer-ZK-EPO conjugate, particle or composition described herein, e.g., a polymer-ZK-EPO conjugate comprising a ZK-EPO molecule, coupled, e.g., via a linker, to a polymer described herein. In an embodiment, the polymer-agent conjugate comprises a ZK-EPO molecule, coupled via a linker shown in FIG. 1 to a polymer, e.g., a polymer described herein. In an embodiment, the polymer-agent conjugate is a polymer-ZK-EPO conjugate shown in FIG. 1. In one embodiment, the polymer-ZK-EPO conjugate, particle or composition is administered in combination with one or more additional agent, e.g., one or more chemotherapeutic agent described herein. In one embodiment, at least an additional dose of the polymer-ZK-EPO conjugate, particle or composition is administered, e.g., at a dose and/or dosing schedule described herein.

In one aspect, the disclosure features a method of treating a subject, e.g., a human, having a proliferative disorder, e.g., cancer, the method comprising:

selecting a subject having a proliferative disease who has received an epothilone (e.g., ixabepilone, epothilone B, epothilone D, BMS310705, dehydelone or ZK-EPO) and has a platelet count less than a standard; and

administering a polymer-agent conjugate, particle or composition, e.g., a polymer-agent conjugate, particle or composition described herein, to the subject in an amount effective to treat the proliferative disorder, to thereby treat the disorder.

In an embodiment, the polymer-agent conjugate comprises an anticancer agent such as an epothilone, coupled, e.g., via a linker, to a polymer described herein. In an embodiment, the polymer-agent conjugate comprises an epothilone molecule, coupled via a linker shown in FIG. 1 to a polymer, e.g., a polymer described herein. In an embodiment, the polymer-agent conjugate is a polymer-epothilone conjugate shown in FIG. 1.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the polymer-ixabepilone conjugate, particle or composition is administered in combination with one or more additional agent, e.g., one or more chemotherapeutic agent described herein. In one embodiment, at least an additional dose of the polymer-ixabepilone conjugate, particle or composition is administered, e.g., at a dose and/or dosing schedule described herein.

In one embodiment, the polymer-agent conjugate, particle or composition includes a polymer-epothilone B conjugate, particle or composition, e.g., a polymer-epothilone B conjugate, particle or composition described herein, e.g., a polymer-epothilone B conjugate comprising an epothilone B molecule, coupled, e.g., via a linker, to a polymer described herein. In an embodiment, the polymer-agent conjugate comprises an epothilone B molecule, coupled via a linker shown in FIG. 1 to a polymer, e.g., a polymer described herein. In an embodiment, the polymer-agent conjugate is a polymer-epothilone B conjugate shown in FIG. 1. In one embodiment, the polymer-epothilone B conjugate, particle or composition is administered in combination with one or more additional agent, e.g., one or more chemotherapeutic agent described herein. In one embodiment, at least an additional dose of the polymer-epothilone B conjugate, particle or composition is administered, e.g., at a dose and/or dosing schedule described herein.

In one embodiment, the polymer-agent conjugate, particle or composition includes a polymer-epothilone D conjugate, particle or composition, e.g., a polymer-epothilone D conjugate, particle or composition described herein, e.g., a polymer-epothilone D conjugate comprising an epothilone D molecule, coupled, e.g., via a linker, to a polymer described herein. In an embodiment, the polymer-agent conjugate comprises an epothilone D molecule, coupled via a linker shown in FIG. 1 to a polymer, e.g., a polymer described herein. In an embodiment, the polymer-agent conjugate is a polymer-epothilone D conjugate shown in FIG. 1. In one embodiment, the polymer-epothilone D conjugate, particle or composition is administered in combination with one or more additional agent, e.g., one or more chemotherapeutic agent described herein. In one embodiment, at least an additional dose of the polymer-epothilone D conjugate, particle or composition is administered, e.g., at a dose and/or dosing schedule described herein.

In one embodiment, the polymer-agent conjugate, particle or composition includes a polymer-BMS310705 conjugate, particle or composition, e.g., a polymer-BMS310705 conjugate, particle or composition described herein, e.g., a polymer-BMS310705 conjugate comprising a BMS310705 molecule, coupled, e.g., via a linker, to a polymer described herein. In an embodiment, the polymer-agent conjugate comprises a BMS310705 molecule, coupled via a linker shown in FIG. 1 to a polymer, e.g., a polymer described herein. In an embodiment, the polymer-agent conjugate is a polymer-BMS310705 conjugate shown in FIG. 1. In one embodiment, the polymer-BMS310705 conjugate, particle or composition is administered in combination with one or more additional agent, e.g., one or more chemotherapeutic agent described herein. In one embodiment, at least an additional dose of the polymer-BMS310705 conjugate, particle or composition is administered, e.g., at a dose and/or dosing schedule described herein.

In one embodiment, the polymer-agent conjugate, particle or composition includes a polymer-dehydelone conjugate, particle or composition, e.g., a polymer-dehydelone conjugate, particle or composition described herein, e.g., a polymer-dehydelone conjugate comprising a dehydelone molecule, coupled, e.g., via a linker, to a polymer described herein. In an embodiment, the polymer-agent conjugate comprises a dehydelone molecule, coupled via a linker shown in FIG. 1 to a polymer, e.g., a polymer described herein. In an embodiment, the polymer-agent conjugate is a polymer-dehydelone conjugate shown in FIG. 1. In one embodiment, the polymer-dehydelone conjugate, particle or composition is administered in combination with one or more additional agent, e.g., one or more chemotherapeutic agent described herein. In one embodiment, at least an additional dose of the polymer-dehydelone conjugate, particle or composition is administered, e.g., at a dose and/or dosing schedule described herein.

In one embodiment, the polymer-agent conjugate, particle or composition is a polymer-ZK-EPO conjugate, particle or composition, e.g., a polymer-ZK-EPO conjugate, particle or composition described herein, e.g., a polymer-ZK-EPO conjugate comprising a ZK-EPO molecule, coupled, e.g., via a linker, to a polymer described herein. In an embodiment, the polymer-agent conjugate comprises a ZK-EPO molecule, coupled via a linker shown in FIG. 1 to a polymer, e.g., a polymer described herein. In an embodiment, the polymer-agent conjugate is a polymer-ZK-EPO conjugate shown in FIG. 1. In one embodiment, the polymer-ZK-EPO conjugate, particle or composition is administered in combination with one or more additional agent, e.g., one or more chemotherapeutic agent described herein. In one embodiment, at least an additional dose of the polymer-ZK-EPO conjugate, particle or composition is administered, e.g., at a dose and/or dosing schedule described herein.

In one embodiment, the standard is a platelet count below or equal to 50×10³ platelets/mm³. In some embodiments, the standard is platelet count prior to receiving an epothilone treatment. In one embodiment, the standard is a decrease from the mean platelet count prior to initiation of the treatment with an epothilone, e.g., by at least 20%, 30%, 40% or 50%.

In one aspect, the disclosure features a method of identifying a subject, e.g., a human, having a proliferative disorder, e.g., cancer, for treatment with a polymer-agent conjugate, particle or composition, e.g., a polymer-agent conjugate, particle or composition described herein, the method comprising:

identifying a subject having a proliferative disorder who has received an epothilone (e.g., ixabepilone, epothilone B, epothilone D, BMS310705, dehydelone or ZK-EPO) and has a neutrophil count less than a standard; and

identifying the subject as suitable for treatment with a polymer-agent conjugate, particle or composition, e.g., a polymer-agent conjugate, particle or composition described herein.

In an embodiment, the polymer-agent conjugate comprises an anticancer agent such as an epothilone, coupled, e.g., via a linker, to a polymer described herein. In an embodiment, the polymer-agent conjugate comprises an epothilone molecule, coupled via a linker shown in FIG. 1 to a polymer, e.g., a polymer described herein. In an embodiment, the polymer-agent conjugate is a polymer-epothilone conjugate shown in FIG. 1.

In one embodiment, the method further comprising administering a polymer-agent conjugate, particle or composition, e.g., a polymer-agent conjugate, particle or composition described herein in an amount effective to treat the disorder.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the polymer-ixabepilone conjugate, particle or composition is administered in combination with one or more additional agent, e.g., one or more chemotherapeutic agent described herein. In one embodiment, at least an additional dose of the polymer-ixabepilone conjugate, particle or composition is administered, e.g., at a dose and/or dosing schedule described herein.

In one embodiment, the polymer-agent conjugate, particle or composition includes a polymer-epothilone B conjugate, particle or composition, e.g., a polymer-epothilone B conjugate, particle or composition described herein, e.g., a polymer-epothilone B conjugate comprising an epothilone B molecule, coupled, e.g., via a linker, to a polymer described herein. In an embodiment, the polymer-agent conjugate comprises an epothilone B molecule, coupled via a linker shown in FIG. 1 to a polymer, e.g., a polymer described herein. In an embodiment, the polymer-agent conjugate is a polymer-epothilone B conjugate shown in FIG. 1. In one embodiment, the polymer-epothilone B conjugate, particle or composition is administered in combination with one or more additional agent, e.g., one or more chemotherapeutic agent described herein. In one embodiment, at least an additional dose of the polymer-epothilone B conjugate, particle or composition is administered, e.g., at a dose and/or dosing schedule described herein.

In one embodiment, the polymer-agent conjugate, particle or composition includes a polymer-epothilone D conjugate, particle or composition, e.g., a polymer-epothilone D conjugate, particle or composition described herein, e.g., a polymer-epothilone D conjugate comprising an epothilone D molecule, coupled, e.g., via a linker, to a polymer described herein. In an embodiment, the polymer-agent conjugate comprises an epothilone D molecule, coupled via a linker shown in FIG. 1 to a polymer, e.g., a polymer described herein. In an embodiment, the polymer-agent conjugate is a polymer-epothilone D conjugate shown in FIG. 1. In one embodiment, the polymer-epothilone D conjugate, particle or composition is administered in combination with one or more additional agent, e.g., one or more chemotherapeutic agent described herein. In one embodiment, at least an additional dose of the polymer-epothilone D conjugate, particle or composition is administered, e.g., at a dose and/or dosing schedule described herein.

In one embodiment, the polymer-agent conjugate, particle or composition includes a polymer-BMS310705 conjugate, particle or composition, e.g., a polymer-BMS310705 conjugate, particle or composition described herein, e.g., a polymer-BMS310705 conjugate comprising a BMS310705 molecule, coupled, e.g., via a linker, to a polymer described herein. In an embodiment, the polymer-agent conjugate comprises a BMS310705 molecule, coupled via a linker shown in FIG. 1 to a polymer, e.g., a polymer described herein. In an embodiment, the polymer-agent conjugate is a polymer-BMS310705 conjugate shown in FIG. 1. In one embodiment, the polymer-BMS310705 conjugate, particle or composition is administered in combination with one or more additional agent, e.g., one or more chemotherapeutic agent described herein. In one embodiment, at least an additional dose of the polymer-BMS310705 conjugate, particle or composition is administered, e.g., at a dose and/or dosing schedule described herein.

In one embodiment, the polymer-agent conjugate, particle or composition includes a polymer-dehydelone conjugate, particle or composition, e.g., a polymer-dehydelone conjugate, particle or composition described herein, e.g., a polymer-dehydelone conjugate comprising a dehydelone molecule, coupled, e.g., via a linker, to a polymer described herein. In an embodiment, the polymer-agent conjugate comprises a dehydelone molecule, coupled via a linker shown in FIG. 1 to a polymer, e.g., a polymer described herein. In an embodiment, the polymer-agent conjugate is a polymer-dehydelone conjugate shown in FIG. 1. In one embodiment, the polymer-dehydelone conjugate, particle or composition is administered in combination with one or more additional agent, e.g., one or more chemotherapeutic agent described herein. In one embodiment, at least an additional dose of the polymer-dehydelone conjugate, particle or composition is administered, e.g., at a dose and/or dosing schedule described herein.

In one embodiment, the polymer-agent conjugate, particle or composition is a polymer-ZK-EPO conjugate, particle or composition, e.g., a polymer-ZK-EPO conjugate, particle or composition described herein, e.g., a polymer-ZK-EPO conjugate comprising a ZK-EPO molecule, coupled, e.g., via a linker, to a polymer described herein. In an embodiment, the polymer-agent conjugate comprises a ZK-EPO molecule, coupled via a linker shown in FIG. 1 to a polymer, e.g., a polymer described herein. In an embodiment, the polymer-agent conjugate is a polymer-ZK-EPO conjugate shown in FIG. 1. In one embodiment, the polymer-ZK-EPO conjugate, particle or composition is administered in combination with one or more additional agent, e.g., one or more chemotherapeutic agent described herein. In one embodiment, at least an additional dose of the polymer-ZK-EPO conjugate, particle or composition is administered, e.g., at a dose and/or dosing schedule described herein.

In another aspect, the disclosure features a method of treating a subject, e.g., a human, with a proliferative disorder, e.g., cancer, the method comprising:

selecting a subject having a proliferative disease who has received an epothilone (e.g., ixabepilone) and has a neutrophil count less than a standard; and

administering a polymer-agent conjugate, particle or composition, e.g., a polymer-agent conjugate, particle or composition described herein, to the subject in an amount effective to treat the proliferative disorder, to thereby treat the disorder.

In an embodiment, the polymer-agent conjugate comprises an anticancer agent such as an epothilone, coupled, e.g., via a linker, to a polymer described herein. In an embodiment, the polymer-agent conjugate comprises an epothilone molecule, coupled via a linker shown in FIG. 1 to a polymer, e.g., a polymer described herein. In an embodiment, the polymer-agent conjugate is a polymer-epothilone conjugate shown in FIG. 1.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the polymer-ixabepilone conjugate, particle or composition is administered in combination with one or more additional agent, e.g., one or more chemotherapeutic agent described herein. In one embodiment, at least an additional dose of the polymer-ixabepilone conjugate, particle or composition is administered, e.g., at a dose and/or dosing schedule described herein.

In one embodiment, the polymer-agent conjugate, particle or composition includes a polymer-epothilone B conjugate, particle or composition, e.g., a polymer-epothilone B conjugate, particle or composition described herein, e.g., a polymer-epothilone B conjugate comprising an epothilone B molecule, coupled, e.g., via a linker, to a polymer described herein. In an embodiment, the polymer-agent conjugate comprises an epothilone B molecule, coupled via a linker shown in FIG. 1 to a polymer, e.g., a polymer described herein. In an embodiment, the polymer-agent conjugate is a polymer-epothilone B conjugate shown in FIG. 1. In one embodiment, the polymer-epothilone B conjugate, particle or composition is administered in combination with one or more additional agent, e.g., one or more chemotherapeutic agent described herein. In one embodiment, at least an additional dose of the polymer-epothilone B conjugate, particle or composition is administered, e.g., at a dose and/or dosing schedule described herein.

In one embodiment, the polymer-agent conjugate, particle or composition includes a polymer-epothilone D conjugate, particle or composition, e.g., a polymer-epothilone D conjugate, particle or composition described herein, e.g., a polymer-epothilone D conjugate comprising an epothilone D molecule, coupled, e.g., via a linker, to a polymer described herein. In an embodiment, the polymer-agent conjugate comprises an epothilone D molecule, coupled via a linker shown in FIG. 1 to a polymer, e.g., a polymer described herein. In an embodiment, the polymer-agent conjugate is a polymer-epothilone D conjugate shown in FIG. 1. In one embodiment, the polymer-epothilone D conjugate, particle or composition is administered in combination with one or more additional agent, e.g., one or more chemotherapeutic agent described herein. In one embodiment, at least an additional dose of the polymer-epothilone D conjugate, particle or composition is administered, e.g., at a dose and/or dosing schedule described herein.

In one embodiment, the polymer-agent conjugate, particle or composition includes a polymer-BMS310705 conjugate, particle or composition, e.g., a polymer-BMS310705 conjugate, particle or composition described herein, e.g., a polymer-BMS310705 conjugate comprising a BMS310705 molecule, coupled, e.g., via a linker, to a polymer described herein. In an embodiment, the polymer-agent conjugate comprises a BMS310705 molecule, coupled via a linker shown in FIG. 1 to a polymer, e.g., a polymer described herein. In an embodiment, the polymer-agent conjugate is a polymer-BMS310705 conjugate shown in FIG. 1. In one embodiment, the polymer-BMS310705 conjugate, particle or composition is administered in combination with one or more additional agent, e.g., one or more chemotherapeutic agent described herein. In one embodiment, at least an additional dose of the polymer-BMS310705 conjugate, particle or composition is administered, e.g., at a dose and/or dosing schedule described herein.

In one embodiment, the polymer-agent conjugate, particle or composition includes a polymer-dehydelone conjugate, particle or composition, e.g., a polymer-dehydelone conjugate, particle or composition described herein, e.g., a polymer-dehydelone conjugate comprising a dehydelone molecule, coupled, e.g., via a linker, to a polymer described herein. In an embodiment, the polymer-agent conjugate comprises a dehydelone molecule, coupled via a linker shown in FIG. 1 to a polymer, e.g., a polymer described herein. In an embodiment, the polymer-agent conjugate is a polymer-dehydelone conjugate shown in FIG. 1. In one embodiment, the polymer-dehydelone conjugate, particle or composition is administered in combination with one or more additional agent, e.g., one or more chemotherapeutic agent described herein. In one embodiment, at least an additional dose of the polymer-dehydelone conjugate, particle or composition is administered, e.g., at a dose and/or dosing schedule described herein.

In one embodiment, the polymer-agent conjugate, particle or composition is a polymer-ZK-EPO conjugate, particle or composition, e.g., a polymer-ZK-EPO conjugate, particle or composition described herein, e.g., a polymer-ZK-EPO conjugate comprising a ZK-EPO molecule, coupled, e.g., via a linker, to a polymer described herein. In an embodiment, the polymer-agent conjugate comprises a ZK-EPO molecule, coupled via a linker shown in FIG. 1 to a polymer, e.g., a polymer described herein. In an embodiment, the polymer-agent conjugate is a polymer-ZK-EPO conjugate shown in FIG. 1. In one embodiment, the polymer-ZK-EPO conjugate, particle or composition is administered in combination with one or more additional agent, e.g., one or more chemotherapeutic agent described herein. In one embodiment, at least an additional dose of the polymer-ZK-EPO conjugate, particle or composition is administered, e.g., at a dose and/or dosing schedule described herein.

In one embodiment, the standard is a neutrophil count below or equal to 1500 cells/mm³. In some embodiments, the standard is based on a neutrophil count prior to receiving an epothilone treatment, e.g., mean neutrophil count decreased from the mean neutrophil count prior to treatment with the epothilone, e.g., by at least 20%, 30%, 40% or 50% after administration of the epothilone.

In one aspect, the disclosure features a method of identifying a subject, e.g., a human, having a proliferative disorder, e.g., cancer, for treatment with a polymer-agent conjugate, particle or composition, e.g., a polymer-agent conjugate, particle or composition described herein, the method comprising:

identifying a subject having a proliferative disorder who has received an epothilone (e.g., ixabepilone) and had one or more symptom of febrile neutropenia; and

identifying the subject as suitable for treatment with a polymer-agent conjugate, particle or composition, e.g., a polymer-agent conjugate, particle or composition described herein.

In an embodiment, the polymer-agent conjugate comprises an anticancer agent such as an epothilone, coupled, e.g., via a linker, to a polymer described herein. In an embodiment, the polymer-agent conjugate comprises an epothilone molecule, coupled via a linker shown in FIG. 1 to a polymer, e.g., a polymer described herein. In an embodiment, the polymer-agent conjugate is a polymer-epothilone conjugate shown in FIG. 1.

In one embodiment, the method further comprises administering a polymer-agent conjugate, particle or composition, e.g., a polymer-agent conjugate, particle or composition described herein, in an amount effective to treat the disorder.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the polymer-ixabepilone conjugate, particle or composition is administered in combination with one or more additional agent, e.g., one or more chemotherapeutic agent described herein. In one embodiment, at least an additional dose of the polymer-ixabepilone conjugate, particle or composition is administered, e.g., at a dose and/or dosing schedule described herein.

In one embodiment, the polymer-agent conjugate, particle or composition includes a polymer-epothilone B conjugate, particle or composition, e.g., a polymer-epothilone B conjugate, particle or composition described herein, e.g., a polymer-epothilone B conjugate comprising an epothilone B molecule, coupled, e.g., via a linker, to a polymer described herein. In an embodiment, the polymer-agent conjugate comprises an epothilone B molecule, coupled via a linker shown in FIG. 1 to a polymer, e.g., a polymer described herein. In an embodiment, the polymer-agent conjugate is a polymer-epothilone B conjugate shown in FIG. 1. In one embodiment, the polymer-epothilone B conjugate, particle or composition is administered in combination with one or more additional agent, e.g., one or more chemotherapeutic agent described herein. In one embodiment, at least an additional dose of the polymer-epothilone B conjugate, particle or composition is administered, e.g., at a dose and/or dosing schedule described herein.

In one embodiment, the polymer-agent conjugate, particle or composition includes a polymer-epothilone D conjugate, particle or composition, e.g., a polymer-epothilone D conjugate, particle or composition described herein, e.g., a polymer-epothilone D conjugate comprising an epothilone D molecule, coupled, e.g., via a linker, to a polymer described herein. In an embodiment, the polymer-agent conjugate comprises an epothilone D molecule, coupled via a linker shown in FIG. 1 to a polymer, e.g., a polymer described herein. In an embodiment, the polymer-agent conjugate is a polymer-epothilone D conjugate shown in FIG. 1. In one embodiment, the polymer-epothilone D conjugate, particle or composition is administered in combination with one or more additional agent, e.g., one or more chemotherapeutic agent described herein. In one embodiment, at least an additional dose of the polymer-epothilone D conjugate, particle or composition is administered, e.g., at a dose and/or dosing schedule described herein.

In one embodiment, the polymer-agent conjugate, particle or composition includes a polymer-BMS310705 conjugate, particle or composition, e.g., a polymer-BMS310705 conjugate, particle or composition described herein, e.g., a polymer-BMS310705 conjugate comprising a BMS310705 molecule, coupled, e.g., via a linker, to a polymer described herein. In an embodiment, the polymer-agent conjugate comprises a BMS310705 molecule, coupled via a linker shown in FIG. 1 to a polymer, e.g., a polymer described herein. In an embodiment, the polymer-agent conjugate is a polymer-BMS310705 conjugate shown in FIG. 1. In one embodiment, the polymer-BMS310705 conjugate, particle or composition is administered in combination with one or more additional agent, e.g., one or more chemotherapeutic agent described herein. In one embodiment, at least an additional dose of the polymer-BMS310705 conjugate, particle or composition is administered, e.g., at a dose and/or dosing schedule described herein.

In one embodiment, the polymer-agent conjugate, particle or composition includes a polymer-dehydelone conjugate, particle or composition, e.g., a polymer-dehydelone conjugate, particle or composition described herein, e.g., a polymer-dehydelone conjugate comprising a dehydelone molecule, coupled, e.g., via a linker, to a polymer described herein. In an embodiment, the polymer-agent conjugate comprises a dehydelone molecule, coupled via a linker shown in FIG. 1 to a polymer, e.g., a polymer described herein. In an embodiment, the polymer-agent conjugate is a polymer-dehydelone conjugate shown in FIG. 1. In one embodiment, the polymer-dehydelone conjugate, particle or composition is administered in combination with one or more additional agent, e.g., one or more chemotherapeutic agent described herein. In one embodiment, at least an additional dose of the polymer-dehydelone conjugate, particle or composition is administered, e.g., at a dose and/or dosing schedule described herein.

In one embodiment, the polymer-agent conjugate, particle or composition is a polymer-ZK-EPO conjugate, particle or composition, e.g., a polymer-ZK-EPO conjugate, particle or composition described herein, e.g., a polymer-ZK-EPO conjugate comprising a ZK-EPO molecule, coupled, e.g., via a linker, to a polymer described herein. In an embodiment, the polymer-agent conjugate comprises a ZK-EPO molecule, coupled via a linker shown in FIG. 1 to a polymer, e.g., a polymer described herein. In an embodiment, the polymer-agent conjugate is a polymer-ZK-EPO conjugate shown in FIG. 1. In one embodiment, the polymer-ZK-EPO conjugate, particle or composition is administered in combination with one or more additional agent, e.g., one or more chemotherapeutic agent described herein. In one embodiment, at least an additional dose of the polymer-ZK-EPO conjugate, particle or composition is administered, e.g., at a dose and/or dosing schedule described herein.

In one embodiment, the symptom of febrile neutropenia is one or more of: fever, infection and a low neutrophil count in the blood.

In one aspect, the disclosure features a method of treating a subject, e.g., a human, with a proliferative disorder, e.g., cancer, the method comprising:

selecting a subject having a proliferative disease who has received an epothilone (e.g., ixabepilone, epothilone B, epothilone D, BMS 310705, dehydelone or ZK-EPO) and had one or more symptom of febrile neutropenia; and

administering a polymer-agent conjugate, particle or composition, e.g., a polymer-agent conjugate, particle or composition described herein, to the subject in an amount effective to treat the proliferative disorder, to thereby treat the disorder.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the polymer-ixabepilone conjugate, particle or composition is administered in combination with one or more additional agent, e.g., one or more chemotherapeutic agent described herein. In one embodiment, at least an additional dose of the polymer-ixabepilone conjugate, particle or composition is administered, e.g., at a dose and/or dosing schedule described herein.

In one embodiment, the polymer-agent conjugate, particle or composition includes a polymer-epothilone B conjugate, particle or composition, e.g., a polymer-epothilone B conjugate, particle or composition described herein, e.g., a polymer-epothilone B conjugate comprising an epothilone B molecule, coupled, e.g., via a linker, to a polymer described herein. In an embodiment, the polymer-agent conjugate comprises an epothilone B molecule, coupled via a linker shown in FIG. 1 to a polymer, e.g., a polymer described herein. In an embodiment, the polymer-agent conjugate is a polymer-epothilone B conjugate shown in FIG. 1. In one embodiment, the polymer-epothilone B conjugate, particle or composition is administered in combination with one or more additional agent, e.g., one or more chemotherapeutic agent described herein. In one embodiment, at least an additional dose of the polymer-epothilone B conjugate, particle or composition is administered, e.g., at a dose and/or dosing schedule described herein.

In one embodiment, the polymer-agent conjugate, particle or composition includes a polymer-epothilone D conjugate, particle or composition, e.g., a polymer-epothilone D conjugate, particle or composition described herein, e.g., a polymer-epothilone D conjugate comprising an epothilone D molecule, coupled, e.g., via a linker, to a polymer described herein. In an embodiment, the polymer-agent conjugate comprises an epothilone D molecule, coupled via a linker shown in FIG. 1 to a polymer, e.g., a polymer described herein. In an embodiment, the polymer-agent conjugate is a polymer-epothilone D conjugate shown in FIG. 1. In one embodiment, the polymer-epothilone D conjugate, particle or composition is administered in combination with one or more additional agent, e.g., one or more chemotherapeutic agent described herein. In one embodiment, at least an additional dose of the polymer-epothilone D conjugate, particle or composition is administered, e.g., at a dose and/or dosing schedule described herein.

In one embodiment, the polymer-agent conjugate, particle or composition includes a polymer-BMS310705 conjugate, particle or composition, e.g., a polymer-BMS310705 conjugate, particle or composition described herein, e.g., a polymer-BMS310705 conjugate comprising a BMS310705 molecule, coupled, e.g., via a linker, to a polymer described herein. In an embodiment, the polymer-agent conjugate comprises a BMS310705 molecule, coupled via a linker shown in FIG. 1 to a polymer, e.g., a polymer described herein. In an embodiment, the polymer-agent conjugate is a polymer-BMS310705 conjugate shown in FIG. 1. In one embodiment, the polymer-BMS310705 conjugate, particle or composition is administered in combination with one or more additional agent, e.g., one or more chemotherapeutic agent described herein. In one embodiment, at least an additional dose of the polymer-BMS310705 conjugate, particle or composition is administered, e.g., at a dose and/or dosing schedule described herein.

In one embodiment, the polymer-agent conjugate, particle or composition includes a polymer-dehydelone conjugate, particle or composition, e.g., a polymer-dehydelone conjugate, particle or composition described herein, e.g., a polymer-dehydelone conjugate comprising a dehydelone molecule, coupled, e.g., via a linker, to a polymer described herein. In an embodiment, the polymer-agent conjugate comprises a dehydelone molecule, coupled via a linker shown in FIG. 1 to a polymer, e.g., a polymer described herein. In an embodiment, the polymer-agent conjugate is a polymer-dehydelone conjugate shown in FIG. 1. In one embodiment, the polymer-dehydelone conjugate, particle or composition is administered in combination with one or more additional agent, e.g., one or more chemotherapeutic agent described herein. In one embodiment, at least an additional dose of the polymer-dehydelone conjugate, particle or composition is administered, e.g., at a dose and/or dosing schedule described herein.

In one embodiment, the polymer-agent conjugate, particle or composition is a polymer-ZK-EPO conjugate, particle or composition, e.g., a polymer-ZK-EPO conjugate, particle or composition described herein, e.g., a polymer-ZK-EPO conjugate comprising a ZK-EPO molecule, coupled, e.g., via a linker, to a polymer described herein. In an embodiment, the polymer-agent conjugate comprises a ZK-EPO molecule, coupled via a linker shown in FIG. 1 to a polymer, e.g., a polymer described herein. In an embodiment, the polymer-agent conjugate is a polymer-ZK-EPO conjugate shown in FIG. 1. In one embodiment, the polymer-ZK-EPO conjugate, particle or composition is administered in combination with one or more additional agent, e.g., one or more chemotherapeutic agent described herein. In one embodiment, at least an additional dose of the polymer-ZK-EPO conjugate, particle or composition is administered, e.g., at a dose and/or dosing schedule described herein.

In one embodiment, the symptom of febrile neutropenia is one or more of: fever, infection, and a low neutrophil count in the blood.

In another aspect, the disclosure features a method of identifying a subject, e.g., a human, having a proliferative disorder, e.g., cancer, for treatment with a polymer-agent conjugate, particle or composition, e.g., a polymer-agent conjugate, particle or composition described herein, the method comprising:

identifying a subject having a proliferative disorder who has one or more symptom of febrile neutropenia; and

identifying the subject as suitable for treatment with a polymer-agent conjugate, particle or composition, e.g., a polymer-agent conjugate, particle or composition described herein, at a dose of 40 mg/m².

In one embodiment, the method further comprises administering a polymer-agent conjugate, particle or composition, e.g., a polymer-agent conjugate, particle or composition described herein, in an amount effective to treat the disorder.

In an embodiment, the polymer-agent conjugate comprises an anticancer agent such as an epothilone, coupled, e.g., via a linker, to a polymer described herein. In an embodiment, the polymer-agent conjugate comprises an epothilone molecule, coupled via a linker shown in FIG. 1 to a polymer, e.g., a polymer described herein. In an embodiment, the polymer-agent conjugate is a polymer-epothilone conjugate shown in FIG. 1.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the subject is selected for administration of the polymer-ixabepilone conjugate, particle or composition in combination with one or more additional agent, e.g., one or more chemotherapeutic agent described herein. In one embodiment, the subject is selected for administration of at least one additional dose of the polymer-ixabepilone conjugate, particle or composition, e.g., at a dose and/or dosing schedule described herein.

In one embodiment, the symptom of febrile neutropenia is one or more of: fever, infection, and a low neutrophil count in the blood.

In one aspect, the disclosure features a method of treating a subject, e.g., a human, with a proliferative disorder, e.g., cancer, the method comprising:

selecting a subject having a proliferative disease who has one or more symptom of febrile neutropenia; and

administering a polymer-agent conjugate, particle or composition, e.g., a polymer-agent conjugate, particle or composition described herein, to the subject at a dose 40 mg/m², to thereby treat the disorder.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the polymer-ixabepilone conjugate, particle or composition is administered in combination with one or more additional agent, e.g., one or more chemotherapeutic agent described herein. In one embodiment, at least an additional dose of the polymer-ixabepilone conjugate, particle or composition is administered, e.g., at a dose and/or dosing schedule described herein.

In one embodiment, the symptom of febrile neutropenia is one or more of: fever, infection, and a low neutrophil count in the blood.

In another aspect, the disclosure features a method of selecting a subject, e.g., a human, with a proliferative disorder, e.g., cancer, for treatment with a polymer-agent conjugate, particle or composition, e.g., a polymer-agent conjugate, particle or composition described herein, comprising:

determining if a subject with a proliferative disorder, e.g., a cancer, is at risk for or has diarrhea or has experienced diarrhea from treatment with an epothilone, e.g., epothilone B; and

selecting a subject who is at risk for or has diarrhea or has experienced diarrhea from treatment with an epothilone for treatment with a polymer-agent conjugate, particle or composition, e.g., a polymer-agent conjugate, particle or composition described herein.

In an embodiment, the polymer-agent conjugate comprises an anticancer agent such as an epothilone, coupled, e.g., via a linker, to a polymer described herein. In an embodiment, the polymer-agent conjugate comprises an epothilone molecule, coupled via a linker shown in FIG. 1 to a polymer, e.g., a polymer described herein. In an embodiment, the polymer-agent conjugate is a polymer-epothilone conjugate shown in FIG. 1.

In one embodiment, the polymer-agent conjugate, particle or composition includes a polymer-epothilone B conjugate, particle or composition, e.g., a polymer-epothilone B conjugate, particle or composition described herein, e.g., a polymer-epothilone B conjugate comprising an epothilone B molecule, coupled, e.g., via a linker, to a polymer described herein. In an embodiment, the polymer-agent conjugate comprises an epothilone B molecule, coupled via a linker shown in FIG. 1 to a polymer, e.g., a polymer described herein. In an embodiment, the polymer-agent conjugate is a polymer-epothilone B conjugate shown in FIG. 1. In one embodiment, the subject is selected for treatment with the polymer-epothilone B conjugate, particle or composition in combination with one or more additional agent, e.g., one or more chemotherapeutic agent described herein. In one embodiment, the subject is selected for administration of at least an additional dose of the polymer-epothilone B conjugate, particle or composition, e.g., at a dose and/or dosing schedule described herein.

In one embodiment, the polymer-agent conjugate, particle or composition is administered in combination with an anti-diarrheal agent. The anti-diarrheal agent can be, e.g., an opioid (e.g., codeine, oxycodone, Percocet, paregoric, tincture of opium, diphenoxylate, diflenoxin), loperamide, bismuth subsalicylate, lanreotide, vapreotide, motilin antagonists, COX2 inhibitors (e.g., celecoxib), glutamine, thalidomide, a kaolin agent, a pectin agent, a berberine agent, a muscarinic agent, octreotide and a DPP-IV inhibitor.

In one aspect, the disclosure features a method of treating a subject, e.g., a human, having a proliferative disorder, e.g., cancer, comprising:

selecting a subject with a proliferative disorder, e.g., cancer, who is at risk for or has diarrhea or has experienced diarrhea from treatment with an epothilone, e.g., epothilone B; and

administering a polymer-agent conjugate, particle or composition, e.g., a polymer-agent conjugate, particle or composition described herein, to the subject in an amount effective to treat the disorder, to thereby treat the disorder.

In an embodiment, the polymer-agent conjugate comprises an anticancer agent such as an epothilone, coupled, e.g., via a linker, to a polymer described herein. In an embodiment, the polymer-agent conjugate comprises an epothilone molecule, coupled via a linker shown in FIG. 1 to a polymer, e.g., a polymer described herein. In an embodiment, the polymer-agent conjugate is a polymer-epothilone conjugate shown in FIG. 1.

In one embodiment, the polymer-agent conjugate, particle or composition includes a polymer-epothilone B conjugate, particle or composition, e.g., a polymer-epothilone B conjugate, particle or composition described herein, e.g., a polymer-epothilone B conjugate comprising an epothilone B molecule, coupled, e.g., via a linker, to a polymer described herein. In an embodiment, the polymer-agent conjugate comprises an epothilone B molecule, coupled via a linker shown in FIG. 1 to a polymer, e.g., a polymer described herein. In an embodiment, the polymer-agent conjugate is a polymer-epothilone B conjugate shown in FIG. 1. In one embodiment, the polymer-epothilone B conjugate, particle or composition is administered in combination with one or more additional agent, e.g., one or more chemotherapeutic agent described herein. In one embodiment, at least an additional dose of the polymer-epothilone B conjugate, particle or composition is administered, e.g., at a dose and/or dosing schedule described herein.

In one embodiment, the polymer-agent conjugate, particle or composition, is administered in combination with an anti-diarrheal agent. The anti-diarrheal agent can be, e.g., an opioid (e.g., codeine, oxycodone, Percocet, paregoric, tincture of opium, diphenoxylate, diflenoxin), loperamide, bismuth subsalicylate, lanreotide, vapreotide, motilin antagonists, COX2 inhibitors (e.g., celecoxib), glutamine, thalidomide, a kaolin agent, a pectin agent, a berberine agent, a muscarinic agent, octreotide and a DPP-IV inhibitor.

In another aspect, the disclosure features a method of selecting a subject, e.g., a human, with a proliferative disorder, e.g., cancer, for treatment with a polymer-agent conjugate, particle or composition, e.g., a polymer-agent conjugate, particle or composition described herein, comprising:

determining if a subject with a proliferative disorder, e.g., a cancer, has a catheter or port, e.g., an in-dwelling catheter or port; and

selecting a subject who has a catheter or port, e.g., an in-dwelling catheter or port, for treatment with a polymer-agent conjugate, particle or composition, e.g., a polymer-agent conjugate, particle or composition described herein.

In an embodiment, the polymer-agent conjugate comprises an anticancer agent such as an epothilone, coupled, e.g., via a linker, to a polymer described herein. In an embodiment, the polymer-agent conjugate comprises an epothilone molecule, coupled via a linker shown in FIG. 1 to a polymer, e.g., a polymer described herein. In an embodiment, the polymer-agent conjugate is a polymer-epothilone conjugate shown in FIG. 1.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the subject is selected for treatment with the polymer-ixabepilone conjugate, particle or composition in combination with one or more additional agent, e.g., one or more chemotherapeutic agent described herein. In one embodiment, the subject is selected for administration of at least an additional dose of the polymer-ixabepilone conjugate, particle or composition, e.g., at a dose and/or dosing schedule described herein.

In one embodiment, the polymer-agent conjugate, particle or composition includes a polymer-epothilone B conjugate, particle or composition, e.g., a polymer-epothilone B conjugate, particle or composition described herein, e.g., a polymer-epothilone B conjugate comprising an epothilone B molecule, coupled, e.g., via a linker, to a polymer described herein. In an embodiment, the polymer-agent conjugate comprises an epothilone B molecule, coupled via a linker shown in FIG. 1 to a polymer, e.g., a polymer described herein. In an embodiment, the polymer-agent conjugate is a polymer-epothilone B conjugate shown in FIG. 1. In one embodiment, the subject is selected for treatment with the polymer-epothilone B conjugate, particle or composition in combination with one or more additional agent, e.g., one or more chemotherapeutic agent described herein. In one embodiment, the subject is selected for administration of at least an additional dose of the polymer-epothilone B conjugate, particle or composition, e.g., at a dose and/or dosing schedule described herein.

In one embodiment, the polymer-agent conjugate, particle or composition includes a polymer-epothilone D conjugate, particle or composition, e.g., a polymer-epothilone D conjugate, particle or composition described herein, e.g., a polymer-epothilone D conjugate comprising an epothilone D molecule, coupled, e.g., via a linker, to a polymer described herein. In an embodiment, the polymer-agent conjugate comprises an epothilone D molecule, coupled via a linker shown in FIG. 1 to a polymer, e.g., a polymer described herein. In an embodiment, the polymer-agent conjugate is a polymer-epothilone D conjugate shown in FIG. 1. In one embodiment, the subject is selected for treatment with the polymer-epothilone D conjugate, particle or composition in combination with one or more additional agent, e.g., one or more chemotherapeutic agent described herein. In one embodiment, the subject is selected for administration of at least an additional dose of the polymer-epothilone D conjugate, particle or composition, e.g., at a dose and/or dosing schedule described herein.

In one embodiment, the polymer-agent conjugate, particle or composition includes a polymer-BMS310705 conjugate, particle or composition, e.g., a polymer-BMS310705 conjugate, particle or composition described herein, e.g., a polymer-BMS310705 conjugate comprising a BMS310705 molecule, coupled, e.g., via a linker, to a polymer described herein. In an embodiment, the polymer-agent conjugate comprises a BMS310705 molecule, coupled via a linker shown in FIG. 1 to a polymer, e.g., a polymer described herein. In an embodiment, the polymer-agent conjugate is a polymer-BMS310705 conjugate shown in FIG. 1. In one embodiment, the subject is selected for treatment with the polymer-BMS310705 conjugate, particle or composition in combination with one or more additional agent, e.g., one or more chemotherapeutic agent described herein. In one embodiment, the subject is selected for administration of at least an additional dose of the polymer-BMS310705 conjugate, particle or composition, e.g., at a dose and/or dosing schedule described herein.

In one embodiment, the polymer-agent conjugate, particle or composition includes a polymer-dehydelone conjugate, particle or composition, e.g., a polymer-dehydelone conjugate, particle or composition described herein, e.g., a polymer-dehydelone conjugate comprising a dehydelone molecule, coupled, e.g., via a linker, to a polymer described herein. In an embodiment, the polymer-agent conjugate comprises a dehydelone molecule, coupled via a linker shown in FIG. 1 to a polymer, e.g., a polymer described herein. In an embodiment, the polymer-agent conjugate is a polymer-dehydelone conjugate shown in FIG. 1. In one embodiment, the subject is selected for treatment with the polymer-dehydelone conjugate, particle or composition in combination with one or more additional agent, e.g., one or more chemotherapeutic agent described herein. In one embodiment, the subject is selected for administration of at least an additional dose of the polymer-dehydelone conjugate, particle or composition, e.g., at a dose and/or dosing schedule described herein.

In one embodiment, the polymer-agent conjugate, particle or composition includes a polymer-ZK-EPO conjugate, particle or composition, e.g., a polymer-ZK-EPO conjugate, particle or composition described herein, e.g., a polymer-ZK-EPO conjugate comprising a ZK-EPO molecule, coupled, e.g., via a linker, to a polymer described herein. In an embodiment, the polymer-agent conjugate comprises a ZK-EPO molecule, coupled via a linker shown in FIG. 1 to a polymer, e.g., a polymer described herein. In an embodiment, the polymer-agent conjugate is a polymer-ZK-EPO conjugate shown in FIG. 1. In one embodiment, the subject is selected for treatment with the polymer-ZK-EPO conjugate, particle or composition in combination with one or more additional agent, e.g., one or more chemotherapeutic agent described herein. In one embodiment, the subject is selected for administration of at least an additional dose of the polymer-ZK-EPO conjugate, particle or composition, e.g., at a dose and/or dosing schedule described herein.

In one embodiment, the subject is also being administered an anticoagulant such as heparin or warfarin.

In one aspect, the disclosure features a method of treating a subject, e.g., a human, having a proliferative disorder, e.g., cancer, comprising:

selecting a subject with a proliferative disorder, e.g., cancer, who has a catheter or port, e.g., an in-dwelling catheter or port; and

administering a polymer-agent conjugate, particle or composition, e.g., a polymer-agent conjugate, particle or composition described herein, to the subject in an amount effective to treat the disorder, to thereby treat the disorder.

In an embodiment, the polymer-agent conjugate comprises an anticancer agent such as an epothilone, coupled, e.g., via a linker, to a polymer described herein. In an embodiment, the polymer-agent conjugate comprises an epothilone molecule, coupled via a linker shown in FIG. 1 to a polymer, e.g., a polymer described herein. In an embodiment, the polymer-agent conjugate is a polymer-epothilone conjugate shown in FIG. 1.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the polymer-ixabepilone conjugate, particle or composition is administered in combination with one or more additional agent, e.g., one or more chemotherapeutic agent described herein. In one embodiment, at least an additional dose of the polymer-ixabepilone conjugate, particle or composition is administered, e.g., at a dose and/or dosing schedule described herein.

In one embodiment, the polymer-agent conjugate, particle or composition includes a polymer-epothilone B conjugate, particle or composition, e.g., a polymer-epothilone B conjugate, particle or composition described herein, e.g., a polymer-epothilone B conjugate comprising an epothilone B molecule, coupled, e.g., via a linker, to a polymer described herein. In an embodiment, the polymer-agent conjugate comprises an epothilone B molecule, coupled via a linker shown in FIG. 1 to a polymer, e.g., a polymer described herein. In an embodiment, the polymer-agent conjugate is a polymer-epothilone B conjugate shown in FIG. 1. In one embodiment, the polymer-epothilone B conjugate, particle or composition is administered in combination with one or more additional agent, e.g., one or more chemotherapeutic agent described herein. In one embodiment, at least an additional dose of the polymer-epothilone B conjugate, particle or composition is administered, e.g., at a dose and/or dosing schedule described herein.

In one embodiment, the polymer-agent conjugate, particle or composition includes a polymer-epothilone D conjugate, particle or composition, e.g., a polymer-epothilone D conjugate, particle or composition described herein, e.g., a polymer-epothilone D conjugate comprising an epothilone D molecule, coupled, e.g., via a linker, to a polymer described herein. In an embodiment, the polymer-agent conjugate comprises an epothilone D molecule, coupled via a linker shown in FIG. 1 to a polymer, e.g., a polymer described herein. In an embodiment, the polymer-agent conjugate is a polymer-epothilone D conjugate shown in FIG. 1. In one embodiment, the polymer-epothilone D conjugate, particle or composition is administered in combination with one or more additional agent, e.g., one or more chemotherapeutic agent described herein. In one embodiment, at least an additional dose of the polymer-epothilone D conjugate, particle or composition is administered, e.g., at a dose and/or dosing schedule described herein.

In one embodiment, the polymer-agent conjugate, particle or composition includes a polymer-BMS310705 conjugate, particle or composition, e.g., a polymer-BMS310705 conjugate, particle or composition described herein, e.g., a polymer-BMS310705 conjugate comprising a BMS310705 molecule, coupled, e.g., via a linker, to a polymer described herein. In an embodiment, the polymer-agent conjugate comprises a BMS310705 molecule, coupled via a linker shown in FIG. 1 to a polymer, e.g., a polymer described herein. In an embodiment, the polymer-agent conjugate is a polymer-BMS310705 conjugate shown in FIG. 1. In one embodiment, the polymer-BMS310705 conjugate, particle or composition is administered in combination with one or more additional agent, e.g., one or more chemotherapeutic agent described herein. In one embodiment, at least an additional dose of the polymer-BMS310705 conjugate, particle or composition is administered, e.g., at a dose and/or dosing schedule described herein.

In one embodiment, the polymer-agent conjugate, particle or composition includes a polymer-dehydelone conjugate, particle or composition, e.g., a polymer-dehydelone conjugate, particle or composition described herein, e.g., a polymer-dehydelone conjugate comprising a dehydelone molecule, coupled, e.g., via a linker, to a polymer described herein. In an embodiment, the polymer-agent conjugate comprises a dehydelone molecule, coupled via a linker shown in FIG. 1 to a polymer, e.g., a polymer described herein. In an embodiment, the polymer-agent conjugate is a polymer-dehydelone conjugate shown in FIG. 1. In one embodiment, the polymer-dehydelone conjugate, particle or composition is administered in combination with one or more additional agent, e.g., one or more chemotherapeutic agent described herein. In one embodiment, at least an additional dose of the polymer-dehydelone conjugate, particle or composition is administered, e.g., at a dose and/or dosing schedule described herein.

In one embodiment, the polymer-agent conjugate, particle or composition includes a polymer-ZK-EPO conjugate, particle or composition, e.g., a polymer-ZK-EPO conjugate, particle or composition described herein, e.g., a polymer-ZK-EPO conjugate, particle or composition comprising a ZK-EPO molecule, coupled, e.g., via a linker, to a polymer described herein. In an embodiment, the polymer-agent conjugate comprises a ZK-EPO molecule, coupled via a linker shown in FIG. 1 to a polymer, e.g., a polymer described herein. In an embodiment, the polymer-agent conjugate is a polymer-ZK-EPO conjugate shown in FIG. 1. In one embodiment, the polymer-ZK-EPO conjugate, particle or composition is administered in combination with one or more additional agent, e.g., one or more chemotherapeutic agent described herein. In one embodiment, at least an additional dose of the polymer-ZK-EPO conjugate, particle or composition is administered, e.g., at a dose and/or dosing schedule described herein.

In one embodiment, the subject is also administered an anticoagulant such as heparin or warfarin.

In another aspect, the disclosure features a method of selecting a subject, e.g., a human, with a proliferative disorder, e.g., cancer, for treatment with a polymer-agent conjugate, particle or composition, e.g., a polymer-agent conjugate, particle or composition described herein, comprising:

determining if a subject with a proliferative disorder, e.g., a cancer, is at risk for needing an anticoagulant or is currently being administered an anticoagulant; and

selecting a subject who is at risk for needing an anticoagulant or is currently being administered an anticoagulant for treatment with a polymer-agent conjugate, particle or composition, e.g., a polymer-agent conjugate, particle or composition described herein.

In an embodiment, the polymer-agent conjugate comprises an anticancer agent such as an epothilone, coupled, e.g., via a linker, to a polymer described herein. In an embodiment, the polymer-agent conjugate comprises an epothilone molecule, coupled via a linker shown in FIG. 1 to a polymer, e.g., a polymer described herein. In an embodiment, the polymer-agent conjugate is a polymer-epothilone conjugate shown in FIG. 1.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the subject is selected for treatment with the polymer-ixabepilone conjugate, particle or composition in combination with one or more additional agent, e.g., one or more chemotherapeutic agent described herein. In one embodiment, the subject is selected for administration of at least an additional dose of the polymer-ixabepilone conjugate, particle or composition, e.g., at a dose and/or dosing schedule described herein.

In one embodiment, the polymer-agent conjugate, particle or composition includes a polymer-epothilone B conjugate, particle or composition, e.g., a polymer-epothilone B conjugate, particle or composition described herein, e.g., a polymer-epothilone B conjugate comprising an epothilone B molecule, coupled, e.g., via a linker, to a polymer described herein. In an embodiment, the polymer-agent conjugate comprises an epothilone B molecule, coupled via a linker shown in FIG. 1 to a polymer, e.g., a polymer described herein. In an embodiment, the polymer-agent conjugate is a polymer-epothilone B conjugate shown in FIG. 1. In one embodiment, the subject is selected for treatment with the polymer-epothilone B conjugate, particle or composition in combination with one or more additional agent, e.g., one or more chemotherapeutic agent described herein. In one embodiment, the subject is selected for administration of at least an additional dose of the polymer-epothilone B conjugate, particle or composition, e.g., at a dose and/or dosing schedule described herein.

In one embodiment, the polymer-agent conjugate, particle or composition includes a polymer-epothilone D conjugate, particle or composition, e.g., a polymer-epothilone D conjugate, particle or composition described herein, e.g., a polymer-epothilone D conjugate comprising an epothilone D molecule, coupled, e.g., via a linker, to a polymer described herein. In an embodiment, the polymer-agent conjugate comprises an epothilone D molecule, coupled via a linker shown in FIG. 1 to a polymer, e.g., a polymer described herein. In an embodiment, the polymer-agent conjugate is a polymer-epothilone D conjugate shown in FIG. 1. In one embodiment, the subject is selected for treatment with the polymer-epothilone D conjugate, particle or composition in combination with one or more additional agent, e.g., one or more chemotherapeutic agent described herein. In one embodiment, the subject is selected for administration of at least an additional dose of the polymer-epothilone D conjugate, particle or composition, e.g., at a dose and/or dosing schedule described herein.

In one embodiment, the polymer-agent conjugate, particle or composition includes a polymer-BMS310705 conjugate, particle or composition, e.g., a polymer-BMS310705 conjugate, particle or composition described herein, e.g., a polymer-BMS310705 conjugate comprising a BMS310705 molecule, coupled, e.g., via a linker, to a polymer described herein. In an embodiment, the polymer-agent conjugate comprises a BMS310705 molecule, coupled via a linker shown in FIG. 1 to a polymer, e.g., a polymer described herein. In an embodiment, the polymer-agent conjugate is a polymer-BMS310705 conjugate shown in FIG. 1. In one embodiment, the subject is selected for treatment with the polymer-BMS310705 conjugate, particle or composition in combination with one or more additional agent, e.g., one or more chemotherapeutic agent described herein. In one embodiment, the subject is selected for administration of at least an additional dose of the polymer-BMS310705 conjugate, particle or composition, e.g., at a dose and/or dosing schedule described herein.

In one embodiment, the polymer-agent conjugate, particle or composition includes a polymer-dehydelone conjugate, particle or composition, e.g., a polymer-dehydelone conjugate, particle or composition described herein, e.g., a polymer-dehydelone conjugate comprising a dehydelone molecule, coupled, e.g., via a linker, to a polymer described herein. In an embodiment, the polymer-agent conjugate comprises a dehydelone molecule, coupled via a linker shown in FIG. 1 to a polymer, e.g., a polymer described herein. In an embodiment, the polymer-agent conjugate is a polymer-dehydelone conjugate shown in FIG. 1. In one embodiment, the subject is selected for treatment with the polymer-dehydelone conjugate, particle or composition in combination with one or more additional agent, e.g., one or more chemotherapeutic agent described herein. In one embodiment, the subject is selected for administration of at least an additional dose of the polymer-dehydelone conjugate, particle or composition, e.g., at a dose and/or dosing schedule described herein.

In one embodiment, the polymer-agent conjugate, particle or composition includes a polymer-ZK-EPO conjugate, particle or composition, e.g., a polymer-ZK-EPO conjugate, particle or composition described herein, e.g., a polymer-ZK-EPO conjugate comprising a ZK-EPO molecule, coupled, e.g., via a linker, to a polymer described herein. In an embodiment, the polymer-agent conjugate comprises a ZK-EPO molecule, coupled via a linker shown in FIG. 1 to a polymer, e.g., a polymer described herein. In an embodiment, the polymer-agent conjugate is a polymer-ZK-EPO conjugate shown in FIG. 1. In one embodiment, the subject is selected for treatment with the polymer-ZK-EPO conjugate, particle or composition in combination with one or more additional agent, e.g., one or more chemotherapeutic agent described herein. In one embodiment, the subject is selected for administration of at least an additional dose of the polymer-ZK-EPO conjugate, particle or composition, e.g., at a dose and/or dosing schedule described herein.

In one embodiment, the anticoagulant is a heparin or warfarin.

In one aspect, the disclosure features a method of treating a subject, e.g., a human, having a proliferative disorder, e.g., cancer, comprising:

selecting a subject with a proliferative disorder, e.g., cancer, who is at risk for needing an anticoagulant or is currently being administered an anticoagulant; and

administering a polymer-agent conjugate, particle or composition, e.g., a polymer-agent conjugate, particle or composition described herein, to the subject in an amount effective to treat the disorder, to thereby treat the disorder.

In an embodiment, the polymer-agent conjugate comprises an anticancer agent such as an epothilone, coupled, e.g., via a linker, to a polymer described herein. In an embodiment, the polymer-agent conjugate comprises an epothilone molecule, coupled via a linker shown in FIG. 1 to a polymer, e.g., a polymer described herein. In an embodiment, the polymer-agent conjugate is a polymer-epothilone conjugate shown in FIG. 1.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the polymer-ixabepilone conjugate, particle or composition is administered in combination with one or more additional agent, e.g., one or more chemotherapeutic agent described herein. In one embodiment, at least an additional dose of the polymer-ixabepilone conjugate, particle or composition is administered, e.g., at a dose and/or dosing schedule described herein.

In one embodiment, the polymer-agent conjugate, particle or composition includes a polymer-epothilone B conjugate, particle or composition, e.g., a polymer-epothilone B conjugate, particle or composition described herein, e.g., a polymer-epothilone B conjugate comprising an epothilone B molecule, coupled, e.g., via a linker, to a polymer described herein. In an embodiment, the polymer-agent conjugate comprises an epothilone B molecule, coupled via a linker shown in FIG. 1 to a polymer, e.g., a polymer described herein. In an embodiment, the polymer-agent conjugate is a polymer-epothilone B conjugate shown in FIG. 1. In one embodiment, the polymer-epothilone B conjugate, particle or composition is administered in combination with one or more additional agent, e.g., one or more chemotherapeutic agent described herein. In one embodiment, at least an additional dose of the polymer-epothilone B conjugate, particle or composition is administered, e.g., at a dose and/or dosing schedule described herein.

In one embodiment, the polymer-agent conjugate, particle or composition includes a polymer-epothilone D conjugate, particle or composition, e.g., a polymer-epothilone D conjugate, particle or composition described herein, e.g., a polymer-epothilone D conjugate comprising an epothilone D molecule, coupled, e.g., via a linker, to a polymer described herein. In an embodiment, the polymer-agent conjugate comprises an epothilone D molecule, coupled via a linker shown in FIG. 1 to a polymer, e.g., a polymer described herein. In an embodiment, the polymer-agent conjugate is a polymer-epothilone D conjugate shown in FIG. 1. In one embodiment, the polymer-epothilone D conjugate, particle or composition is administered in combination with one or more additional agent, e.g., one or more chemotherapeutic agent described herein. In one embodiment, at least an additional dose of the polymer-epothilone D conjugate, particle or composition is administered, e.g., at a dose and/or dosing schedule described herein.

In one embodiment, the polymer-agent conjugate, particle or composition includes a polymer-BMS310705 conjugate, particle or composition, e.g., a polymer-BMS310705 conjugate, particle or composition described herein, e.g., a polymer-BMS310705 conjugate comprising a BMS310705 molecule, coupled, e.g., via a linker, to a polymer described herein. In an embodiment, the polymer-agent conjugate comprises a BMS310705 molecule, coupled via a linker shown in FIG. 1 to a polymer, e.g., a polymer described herein. In an embodiment, the polymer-agent conjugate is a polymer-BMS310705 conjugate shown in FIG. 1. In one embodiment, the polymer-BMS310705 conjugate, particle or composition is administered in combination with one or more additional agent, e.g., one or more chemotherapeutic agent described herein. In one embodiment, at least an additional dose of the polymer-BMS310705 conjugate, particle or composition is administered, e.g., at a dose and/or dosing schedule described herein.

In one embodiment, the polymer-agent conjugate, particle or composition includes a polymer-dehydelone conjugate, particle or composition, e.g., a polymer-dehydelone conjugate, particle or composition described herein, e.g., a polymer-dehydelone conjugate comprising a dehydelone molecule, coupled, e.g., via a linker, to a polymer described herein. In an embodiment, the polymer-agent conjugate comprises a dehydelone molecule, coupled via a linker shown in FIG. 1 to a polymer, e.g., a polymer described herein. In an embodiment, the polymer-agent conjugate is a polymer-dehydelone conjugate shown in FIG. 1. In one embodiment, the polymer-dehydelone conjugate, particle or composition is administered in combination with one or more additional agent, e.g., one or more chemotherapeutic agent described herein. In one embodiment, at least an additional dose of the polymer-dehydelone conjugate, particle or composition is administered, e.g., at a dose and/or dosing schedule described herein.

In one embodiment, the polymer-agent conjugate, particle or composition is a polymer-ZK-EPO conjugate, particle or composition, e.g., a polymer-ZK-EPO conjugate, particle or composition described herein, e.g., a polymer-ZK-EPO conjugate comprising a ZK-EPO molecule, coupled, e.g., via a linker, to a polymer described herein. In an embodiment, the polymer-agent conjugate comprises a ZK-EPO molecule, coupled via a linker shown in FIG. 1 to a polymer, e.g., a polymer described herein. In an embodiment, the polymer-agent conjugate is a polymer-ZK-EPO conjugate shown in FIG. 1. In one embodiment, the polymer-ZK-EPO conjugate, particle or composition is administered in combination with one or more additional agent, e.g., one or more chemotherapeutic agent described herein. In one embodiment, at least an additional dose of the polymer-ZK-EPO conjugate, particle or composition is administered, e.g., at a dose and/or dosing schedule described herein.

In one embodiment, the anticoagulant is a heparin or warfarin.

In another aspect, the disclosure features a method of selecting a subject, e.g., a human, with a proliferative disorder, e.g., cancer, for treatment with a polymer-agent conjugate, particle or composition, e.g., a polymer-agent conjugate, particle or composition described herein, comprising:

determining if a subject with a proliferative disorder, e.g., a cancer, is at risk for needing a hematopoietic growth factor or is currently being administered a hematopoietic growth factor; and

selecting a subject who is at risk for needing a hematopoietic growth factor or is currently being administered a hematopoietic growth factor for treatment with a polymer-agent conjugate, particle or composition, e.g., a polymer-agent conjugate, particle or composition described herein.

In an embodiment, the polymer-agent conjugate comprises an anticancer agent such as an epothilone, coupled, e.g., via a linker, to a polymer described herein. In an embodiment, the polymer-agent conjugate comprises an epothilone molecule, coupled via a linker shown in FIG. 1 to a polymer, e.g., a polymer described herein. In an embodiment, the polymer-agent conjugate is a polymer-epothilone conjugate shown in FIG. 1.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the subject is selected for treatment with the polymer-ixabepilone conjugate, particle or composition in combination with one or more additional agent, e.g., one or more chemotherapeutic agent described herein. In one embodiment, the subject is selected for administration of at least an additional dose of the polymer-ixabepilone conjugate, particle or composition, e.g., at a dose and/or dosing schedule described herein.

In one embodiment, the polymer-agent conjugate, particle or composition includes a polymer-epothilone B conjugate, particle or composition, e.g., a polymer-epothilone B conjugate, particle or composition described herein, e.g., a polymer-epothilone B conjugate comprising an epothilone B molecule, coupled, e.g., via a linker, to a polymer described herein. In an embodiment, the polymer-agent conjugate comprises an epothilone B molecule, coupled via a linker shown in FIG. 1 to a polymer, e.g., a polymer described herein. In an embodiment, the polymer-agent conjugate is a polymer-epothilone B conjugate shown in FIG. 1. In one embodiment, the subject is selected for treatment with the polymer-epothilone B conjugate, particle or composition in combination with one or more additional agent, e.g., one or more chemotherapeutic agent described herein. In one embodiment, the subject is selected for administration of at least an additional dose of the polymer-epothilone B conjugate, particle or composition, e.g., at a dose and/or dosing schedule described herein.

In one embodiment, the polymer-agent conjugate, particle or composition includes a polymer-epothilone D conjugate, particle or composition, e.g., a polymer-epothilone D conjugate, particle or composition described herein, e.g., a polymer-epothilone D conjugate comprising an epothilone D molecule, coupled, e.g., via a linker, to a polymer described herein. In an embodiment, the polymer-agent conjugate comprises an epothilone D molecule, coupled via a linker shown in FIG. 1 to a polymer, e.g., a polymer described herein. In an embodiment, the polymer-agent conjugate is a polymer-epothilone D conjugate shown in FIG. 1. In one embodiment, the subject is selected for treatment with the polymer-epothilone D conjugate, particle or composition in combination with one or more additional agent, e.g., one or more chemotherapeutic agent described herein. In one embodiment, the subject is selected for administration of at least an additional dose of the polymer-epothilone D conjugate, particle or composition, e.g., at a dose and/or dosing schedule described herein.

In one embodiment, the polymer-agent conjugate, particle or composition includes a polymer-BMS310705 conjugate, particle or composition, e.g., a polymer-BMS310705 conjugate, particle or composition described herein, e.g., a polymer-BMS310705 conjugate comprising a BMS310705 molecule, coupled, e.g., via a linker, to a polymer described herein. In an embodiment, the polymer-agent conjugate comprises a BMS310705 molecule, coupled via a linker shown in FIG. 1 to a polymer, e.g., a polymer described herein. In an embodiment, the polymer-agent conjugate is a polymer-BMS310705 conjugate shown in FIG. 1. In one embodiment, the subject is selected for treatment with the polymer-BMS310705 conjugate, particle or composition in combination with one or more additional agent, e.g., one or more chemotherapeutic agent described herein. In one embodiment, the subject is selected for administration of at least an additional dose of the polymer-BMS310705 conjugate, particle or composition, e.g., at a dose and/or dosing schedule described herein.

In one embodiment, the polymer-agent conjugate, particle or composition includes a polymer-dehydelone conjugate, particle or composition, e.g., a polymer-dehydelone conjugate, particle or composition described herein, e.g., a polymer-dehydelone conjugate comprising a dehydelone molecule, coupled, e.g., via a linker, to a polymer described herein. In an embodiment, the polymer-agent conjugate comprises a dehydelone molecule, coupled via a linker shown in FIG. 1 to a polymer, e.g., a polymer described herein. In an embodiment, the polymer-agent conjugate is a polymer-dehydelone conjugate shown in FIG. 1. In one embodiment, the subject is selected for treatment with the polymer-dehydelone conjugate, particle or composition in combination with one or more additional agent, e.g., one or more chemotherapeutic agent described herein. In one embodiment, the subject is selected for administration of at least an additional dose of the polymer-dehydelone conjugate, particle or composition, e.g., at a dose and/or dosing schedule described herein.

In one embodiment, the polymer-agent conjugate, particle or composition includes a polymer-ZK-EPO conjugate, particle or composition, e.g., a polymer-ZK-EPO conjugate, particle or composition described herein, e.g., a polymer-ZK-EPO conjugate comprising a ZK-EPO molecule, coupled, e.g., via a linker, to a polymer described herein. In an embodiment, the polymer-agent conjugate comprises a ZK-EPO molecule, coupled via a linker shown in FIG. 1 to a polymer, e.g., a polymer described herein. In an embodiment, the polymer-agent conjugate is a polymer-ZK-EPO conjugate shown in FIG. 1. In one embodiment, the subject is selected for treatment with the polymer-ZK-EPO conjugate, particle or composition in combination with one or more additional agent, e.g., one or more chemotherapeutic agent described herein. In one embodiment, the subject is selected for administration of at least an additional dose of the polymer-ZK-EPO conjugate, particle or composition, e.g., at a dose and/or dosing schedule described herein.

In one embodiment, the hematopoietic growth factor is a colony stimulating factor such as granulocyte colony stimulating factor (G-CSF) or granulocyte macrophage-colony stimulating factor (GM-CSF).

In one aspect, the disclosure features a method of treating a subject, e.g., a human, having a proliferative disorder, e.g., cancer, comprising:

selecting a subject with a proliferative disorder, e.g., cancer, who is at risk for needing a hematopoietic growth factor or is currently being administered a hematopoietic growth factor; and

administering a polymer-agent conjugate, particle or composition, e.g., a polymer-agent conjugate, particle or composition described herein, to the subject in an amount effective to treat the disorder, to thereby treat the disorder.

In an embodiment, the polymer-agent conjugate comprises an anticancer agent such as an epothilone, coupled, e.g., via a linker, to a polymer described herein. In an embodiment, the polymer-agent conjugate comprises an epothilone molecule, coupled via a linker shown in FIG. 1 to a polymer, e.g., a polymer described herein. In an embodiment, the polymer-agent conjugate is a polymer-epothilone conjugate shown in FIG. 1.

In one embodiment, the polymer-agent conjugate, particle or composition includes a polymer-ixabepilone conjugate, particle or composition, e.g., a polymer-ixabepilone conjugate, particle or composition described herein, e.g., a polymer-ixabepilone conjugate comprising an ixabepilone molecule, coupled, e.g., via a linker, to a polymer described herein. In an embodiment, the polymer-agent conjugate comprises an ixabepilone molecule, coupled via a linker shown in FIG. 1 to a polymer, e.g., a polymer described herein. In an embodiment, the polymer-agent conjugate is a polymer-ixabepilone conjugate shown in FIG. 1. In one embodiment, the polymer-ixabepilone conjugate, particle or composition is administered in combination with one or more additional agent, e.g., one or more chemotherapeutic agent described herein. In one embodiment, at least an additional dose of the polymer-ixabepilone conjugate, particle or composition is administered, e.g., at a dose and/or dosing schedule described herein.

In one embodiment, the polymer-agent conjugate, particle or composition includes a polymer-epothilone B conjugate, particle or composition, e.g., a polymer-epothilone B conjugate, particle or composition described herein, e.g., a polymer-epothilone B conjugate comprising an epothilone B molecule, coupled, e.g., via a linker, to a polymer described herein. In an embodiment, the polymer-agent conjugate comprises an epothilone B molecule, coupled via a linker shown in FIG. 1 to a polymer, e.g., a polymer described herein. In an embodiment, the polymer-agent conjugate is a polymer-epothilone B conjugate shown in FIG. 1. In one embodiment, the polymer-epothilone B conjugate, particle or composition is administered in combination with one or more additional agent, e.g., one or more chemotherapeutic agent described herein. In one embodiment, at least an additional dose of the polymer-epothilone B conjugate, particle or composition is administered, e.g., at a dose and/or dosing schedule described herein.

In one embodiment, the polymer-agent conjugate, particle or composition includes a polymer-epothilone D conjugate, particle or composition, e.g., a polymer-epothilone D conjugate, particle or composition described herein, e.g., a polymer-epothilone D conjugate comprising an epothilone D molecule, coupled, e.g., via a linker, to a polymer described herein. In an embodiment, the polymer-agent conjugate comprises an epothilone D molecule, coupled via a linker shown in FIG. 1 to a polymer, e.g., a polymer described herein. In an embodiment, the polymer-agent conjugate is a polymer-epothilone D conjugate shown in FIG. 1. In one embodiment, the polymer-epothilone D conjugate, particle or composition is administered in combination with one or more additional agent, e.g., one or more chemotherapeutic agent described herein. In one embodiment, at least an additional dose of the polymer-epothilone D conjugate, particle or composition is administered, e.g., at a dose and/or dosing schedule described herein.

In one embodiment, the polymer-agent conjugate, particle or composition includes a polymer-BMS310705 conjugate, particle or composition, e.g., a polymer-BMS310705 conjugate, particle or composition described herein, e.g., a polymer-BMS310705 conjugate comprising a BMS310705 molecule, coupled, e.g., via a linker, to a polymer described herein. In an embodiment, the polymer-agent conjugate comprises a BMS310705 molecule, coupled via a linker shown in FIG. 1 to a polymer, e.g., a polymer described herein. In an embodiment, the polymer-agent conjugate is a polymer-BMS310705 conjugate shown in FIG. 1. In one embodiment, the polymer-BMS310705 conjugate, particle or composition is administered in combination with one or more additional agent, e.g., one or more chemotherapeutic agent described herein. In one embodiment, at least an additional dose of the polymer-BMS310705 conjugate, particle or composition is administered, e.g., at a dose and/or dosing schedule described herein.

In one embodiment, the polymer-agent conjugate, particle or composition includes a polymer-dehydelone conjugate, particle or composition, e.g., a polymer-dehydelone conjugate, particle or composition described herein, e.g., a polymer-dehydelone conjugate comprising a dehydelone molecule, coupled, e.g., via a linker, to a polymer described herein. In an embodiment, the polymer-agent conjugate comprises a dehydelone molecule, coupled via a linker shown in FIG. 1 to a polymer, e.g., a polymer described herein. In an embodiment, the polymer-agent conjugate is a polymer-dehydelone conjugate shown in FIG. 1. In one embodiment, the polymer-dehydelone conjugate, particle or composition is administered in combination with one or more additional agent, e.g., one or more chemotherapeutic agent described herein. In one embodiment, at least an additional dose of the polymer-dehydelone conjugate, particle or composition is administered, e.g., at a dose and/or dosing schedule described herein.

In one embodiment, the polymer-agent conjugate, particle or composition is a polymer-ZK-EPO conjugate, particle or composition, e.g., a polymer-ZK-EPO conjugate, particle or composition described herein, e.g., a polymer-ZK-EPO conjugate comprising a ZK-EPO molecule, coupled, e.g., via a linker, to a polymer described herein. In an embodiment, the polymer-agent conjugate comprises a ZK-EPO molecule, coupled via a linker shown in FIG. 1 to a polymer, e.g., a polymer described herein. In an embodiment, the polymer-agent conjugate is a polymer-ZK-EPO conjugate shown in FIG. 1. In one embodiment, the polymer-ZK-EPO conjugate, particle or composition is administered in combination with one or more additional agent, e.g., one or more chemotherapeutic agent described herein. In one embodiment, at least an additional dose of the polymer-ZK-EPO conjugate, particle or composition is administered, e.g., at a dose and/or dosing schedule described herein.

In one embodiment, the hematopoietic growth factor is a colony stimulating factor such as granulocyte colony stimulating factor (G-CSF) or granulocyte macrophage colony stimulating factor (GM-CSF).

In some embodiments, the polymer-agent conjugate, particle or composition is administered orally, parenterally, or intravenously. In some embodiments, the polymer-agent conjugate, particle or composition is administered to a subject once a day. In some embodiments, the polymer-agent conjugate particle or composition is administered to a subject once a week. In some embodiments, the polymer-agent conjugate, particle or composition is administered to a subject every 21 or every 28 days. In some embodiments, the polymer-agent conjugate, particle or composition is administered over a course of at least about 1 month. In some embodiments, the polymer-agent conjugate, particle or composition is administered over a course of from about 6 months to about 1 year.

In some embodiments, the method further comprises monitoring the subject for one or more toxicities or side effects. In some embodiments, the method further comprises administering at least one additional agent in combination with the polymer-agent conjugate, particle or composition.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 depicts a table of polymer-epothilone conjugates.

FIG. 2 depicts exemplary epothilone structures.

### DETAILED DESCRIPTION

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Polymer-agent conjugates, particles, and compositions are described herein. Also disclosed are dosage forms containing the polymer-agent conjugates, particles and compositions; methods of using the polymer-agent conjugates, particles and compositions (e.g., to treat a disorder); kits including the polymer-agent conjugates, particles and compositions; methods of making the polymer-agent conjugates, particles and compositions; methods of storing the polymer-agent conjugates, particles and compositions; and methods of analyzing the particles.

### DEFINITIONS

The term “ambient conditions,” as used herein, refers to surrounding conditions at about one atmosphere of pressure, 50% relative humidity and about 25° C.

The term “attach,” as used herein with respect to the relationship of a first moiety to a second moiety, e.g., the attachment of an agent to a polymer, refers to the formation of a covalent bond between a first moiety and a second moiety. In the same context, “attachment” refers to the covalent bond. For example, an agent attached to a polymer is an agent covalently bonded to the polymer (e.g., a hydrophobic polymer described herein). The attachment can be a direct attachment, e.g., through a direct bond of the first moiety to the second moiety, or can be through a linker (e.g., through a covalently linked chain of one or more atoms disposed between the first and second moiety). E.g., where an attachment is through a linker, a first moiety (e.g., an agent) is covalently bonded to a linker, which in turn is covalently bonded to a second moiety (e.g., a hydrophobic polymer described herein).

The term “biodegradable” is art-recognized, and includes polymers, compositions and formulations, such as those described herein, that are intended to degrade during use. Biodegradable polymers typically differ from non-biodegradable polymers in that the former may be degraded during use. In certain embodiments, such use involves in vivo use, such as in vivo therapy, and in other certain embodiments, such use involves in vitro use. In general, degradation attributable to biodegradability involves the degradation of a biodegradable polymer into its component subunits, or digestion, e.g., by a biochemical process, of the polymer into smaller, non-polymeric subunits. In certain embodiments, two different types of biodegradation may generally be identified. For example, one type of biodegradation may involve cleavage of bonds (whether covalent or otherwise) in the polymer backbone. In such biodegradation, monomers and oligomers typically result, and even more typically, such biodegradation occurs by cleavage of a bond connecting one or more of subunits of a polymer. In contrast, another type of biodegradation may involve cleavage of a bond (whether covalent or otherwise) internal to a side chain or that connects a side chain to the polymer backbone. In certain embodiments, one or the other or both general types of biodegradation may occur during use of a polymer.

The term “biodegradation,” as used herein, encompasses both general types of biodegradation. The degradation rate of a biodegradable polymer often depends in part on a variety of factors, including the chemical identity of the linkage responsible for any degradation, the molecular weight, crystallinity, biostability, and degree of cross-linking of such polymer, the physical characteristics (e.g., shape and size) of a polymer, assembly of polymers or particle, and the mode and location of administration. For example, a greater molecular weight, a higher degree of crystallinity, and/or a greater biostability, usually lead to slower biodegradation.

An “effective amount” or “an amount effective” refers to an amount of the polymer-agent conjugate, compound or composition which is effective, upon single or multiple dose administrations to a subject, in treating a cell, or curing, alleviating, relieving or improving a symptom of a disorder. An effective amount of the composition may vary according to factors such as the disease state, age, sex, and weight of the individual, and the ability of the compound to elicit a desired response in the individual. An effective amount is also one in which any toxic or detrimental effects of the composition is outweighed by the therapeutically beneficial effects.

The term “embed,” as used herein, refers to the formation of a non-covalent interaction between a first moiety and a second moiety, e.g., an agent and a polymer (e.g., an epothilone and a hydrophobic polymer). An embedded moiety, e.g., an agent embedded in a polymer or a particle, is associated with a polymer or other component of the particle through one or more non-covalent interactions such as van der Waals interactions, hydrophobic interactions, hydrogen bonding, dipole-dipole interactions, ionic interactions, and pi stacking. An embedded moiety has no covalent linkage to the polymer or particle in which it is embedded. An embedded moiety may be completely or partially surrounded by the polymer or particle in which it is embedded.

The term “hydrophilic,” as used herein, refers to a moiety that has a solubility in aqueous solution of at least about 0.05 mg/mL or greater (e.g., at least about 1.0 mg/mL or greater).

The term “hydrophobic,” as used herein, refers to a moiety that can be dissolved in an aqueous solution at physiological ionic strength only to the extent of about 0.05 mg/mL or less (preferably about 0.001 mg/mL or less).

A “hydroxy protecting group” as used herein, is well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitable hydroxy protecting groups include, for example, triethylsilyl (TES), t-butyldimethylsilyl (TBDMS), 2,2,2-trichloroethoxycarbonyl (Troc), and carbobenzyloxy (Cbz).

“Inert atmosphere,” as used herein, refers to an atmosphere composed primarily of an inert gas, which does not chemically react with the polymer-agent conjugates, particles, compositions or mixtures described herein. Examples of inert gases are nitrogen (N₂), helium, and argon.

“Linker,” as used herein, is a moiety having at least two functional groups. One functional group is capable of reacting with a functional group on a polymer

described herein, and a second functional group is capable of reacting with a functional group on agent described herein. In some embodiments the linker has just two functional groups. A linker may have more than two functional groups (e.g., 3, 4, 5, 6, 7, 8, 9, 10 or more functional groups), which may be used, e.g., to link multiple agents to a polymer. Depending on the context, linker can refer to a linker moiety before attachment to either of a first or second moiety (e.g., agent or polymer), after attachment to one moiety but before attachment to a second moiety, or the residue of the linker present after attachment to both the first and second moiety.

The term “lyoprotectant,” as used herein refers to a substance present in a lyophilized preparation. Typically it is present prior to the lyophilization process and persists in the resulting lyophilized preparation. It can be used to protect nanoparticles, liposomes, and/or micelles during lyophilization, for example to reduce or prevent aggregation, particle collapse and/or other types of damage. In an embodiment the lyoprotectant is a cryoprotectant.

In an embodiment the lyoprotectant is a carbohydrate. The term “carbohydrate,” as used herein refers to and encompasses monosaccharides, disaccharides, oligosaccharides and polysaccharides.

In an embodiment, the lyoprotectant is a monosaccharide. The term “monosaccharide,” as used herein refers to a single carbohydrate unit (e.g., a simple sugar) that can not be hydrolyzed to simpler carbohydrate units. Exemplary monosaccharide lyoprotectants include glucose, fructose, galactose, xylose, ribose and the like.

In an embodiment, the lyoprotectant is a disaccharide. The term “disaccharide,” as used herein refers to a compound or a chemical moiety formed by 2 monosaccharide units that are bonded together through a glycosidic linkage, for example through 1-4 linkages or 1-6 linkages. A disaccharide may be hydrolyzed into two monosaccharides. Exemplary disaccharide lyoprotectants include sucrose, trehalose, lactose, maltose and the like.

In an embodiment, the lyoprotectant is an oligosaccharide. The term “oligosaccharide,” as used herein refers to a compound or a chemical moiety formed by 3 to about 15, preferably 3 to about 10 monosaccharide units that are bonded together through glycosidic linkages, for example through 1-4 linkages or 1-6 linkages, to form a linear, branched or cyclic structure. Exemplary oligosaccharide lyoprotectants include cyclodextrins, raffinose, melezitose, maltotriose, stachyose acarbose, and the like. An oligosaccharide can be oxidized or reduced.

In an embodiment, the lyoprotectant is a cyclic oligosaccharide. The term “cyclic oligosaccharide,” as used herein refers to a compound or a chemical moiety formed by 3 to about 15, preferably 6, 7, 8, 9, or 10 monosaccharide units that are bonded together through glycosidic linkages, for example through 1-4 linkages or 1-6 linkages, to form a cyclic structure. Exemplary cyclic oligosaccharide lyoprotectants include cyclic oligosaccharides that are discrete compounds, such as a cyclodextrin, β cyclodextrin, or γ cyclodextrin.

Other exemplary cyclic oligosaccharide lyoprotectants include compounds which include a cyclodextrin moiety in a larger molecular structure, such as a polymer that contains a cyclic oligosaccharide moiety. A cyclic oligosaccharide can be oxidized or reduced, for example, oxidized to dicarbonyl forms. The term “cyclodextrin moiety,” as used herein refers to cyclodextrin (e.g., an α, β, or γ cyclodextrin) radical that is incorporated into, or a part of, a larger molecular structure, such as a polymer. A cyclodextrin moiety can be bonded to one or more other moieties directly, or through an optional linker A cyclodextrin moiety can be oxidized or reduced, for example, oxidized to dicarbonyl forms.

Carbohydrate lyoprotectants, e.g., cyclic oligosaccharide lyoprotectants, can be derivatized carbohydrates. For example, in an embodiment, the lyoprotectant is a derivatized cyclic oligosaccharide, e.g., a derivatized cyclodextrin, e.g., 2 hydroxy propyl-beta cyclodextrin, e.g., partially etherified cyclodextrins (e.g., partially etherified (3 cyclodextrins) disclosed in U.S. Pat. No. 6,407,079, the contents of which are incorporated herein by this reference.

An exemplary lyoprotectant is a polysaccharide. The term “polysaccharide,” as used herein refers to a compound or a chemical moiety formed by at least 16 monosaccharide units that are bonded together through glycosidic linkages, for example through 1-4 linkages or 1-6 linkages, to form a linear, branched or cyclic structure, and includes polymers that comprise polysaccharides as part of their backbone structure. In backbones, the polysaccharide can be linear or cyclic. Exemplary polysaccharide lyoprotectants include glycogen, amylase, cellulose, dextran, maltodextrin and the like.

The term “derivatized carbohydrate,” refers to an entity which differs from the subject non-derivatized carbohydrate by at least one atom. For example, instead of the —OH present on a non-derivatized carbohydrate the derivatized carbohydrate can have —OX, wherein X is other than H. Derivatives may be obtained through chemical functionalization and/or substitution or through de novo synthesis—the term “derivative” implies no process-based limitation.

The term “nanoparticle” is used herein to refer to a material structure whose size in any dimension (e.g., x, y, and z Cartesian dimensions) is less than about 1 micrometer (micron), e.g., less than about 500 nm or less than about 200 nm or less than about 100 nm, and greater than about 5 nm. A nanoparticle can have a variety of geometrical shapes, e.g., spherical, ellipsoidal, etc. The term “nanoparticles” is used as the plural of the term “nanoparticle.”

As used herein, “particle polydispersity index (PDI)” or “particle polydispersity” refers to the width of the particle size distribution. Particle PDI can be calculated from the equation PDI=2a₂/a₁² where a₁ is the 1^{st} Cumulant or moment used to calculate the intensity weighted Z average mean size and a₂ is the 2^{nd} moment used to calculate a parameter defined as the polydispersity index (PdI). A particle PDI of 1 is the theoretical maximum and would be a completely flat size distribution plot. Compositions of particles described herein may have particle PDIs of less than 0.5, less than 0.4, less than 0.3, less than 0.2, or less than 0.1. Particle PDI is further defined in the document “What does polydispersity mean (Malvern)”, which is incorporated herein by reference. (Available at http://www.malvern.com/malvern/kbase.nsf/allbyno/KB000780/$file/FAQ%20-%20What%20does%20polydispersity%20mean.pdf).

“Pharmaceutically acceptable carrier or adjuvant,” as used herein, refers to a carrier or adjuvant that may be administered to a patient, together with a polymer-agent conjugate, particle or composition described herein, and which does not destroy the pharmacological activity thereof and is nontoxic when administered in doses sufficient to deliver a therapeutic amount of the particle. Some examples of materials which can serve as pharmaceutically acceptable carriers include: (1) sugars, such as lactose, glucose, mannitol and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical compositions.

The term “polymer,” as used herein, is given its ordinary meaning as used in the art, i.e., a molecular structure featuring one or more repeat units (monomers), connected by covalent bonds. The repeat units may all be identical, or in some cases, there may be more than one type of repeat unit present within the polymer. In some cases, the polymer is biologically derived, i.e., a biopolymer. Non-limiting examples of biopolymers include peptides or proteins (i.e., polymers of various amino acids), or nucleic acids such as DNA or RNA.

As used herein, “polymer polydispersity index (PDI)” or “polymer polydispersity” refers to the distribution of molecular mass in a given polymer sample. The polymer PDI calculated is the weight average molecular weight divided by the number average molecular weight. It indicates the distribution of individual molecular masses in a batch of polymers. The polymer PDI has a value typically greater than 1, but as the polymer chains approach uniform chain length, the PDI approaches unity (1).

As used herein, the term “prevent” or “preventing” as used in the context of the administration of an agent to a subject, refers to subjecting the subject to a regimen, e.g., the administration of a polymer-agent conjugate, particle or composition, such that the onset of at least one symptom of the disorder is delayed as compared to what would be seen in the absence of the regimen.

The term “prodrug” is intended to encompass compounds that, under physiological conditions, are converted into therapeutically active agents. A common method for making a prodrug is to include selected moieties that are hydrolyzed under physiological conditions to reveal the desired molecule, such as an ester or an amide. In some embodiments, the prodrug is converted by an enzymatic activity of the host animal. Exemplary prodrugs include hexanoate conjugates.

As used herein, the term “subject” is intended to include human and non-human animals. Exemplary human subjects include a human patient having a disorder, e.g., a disorder described herein, or a normal subject. The term “non-human animals” includes all vertebrates, e.g., non-mammals (such as chickens, amphibians, reptiles) and mammals, such as non-human primates, domesticated and/or agriculturally useful animals, e.g., sheep, dog, cat, cow, pig, etc.

As used herein, the term “treat” or “treating” a subject having a disorder refers to subjecting the subject to a regimen, e.g., the administration of a polymer-agent conjugate, particle or composition, such that at least one symptom of the disorder is cured, healed, alleviated, relieved, altered, remedied, ameliorated, or improved. Treating includes administering an amount effective to alleviate, relieve, alter, remedy, ameliorate, improve or affect the disorder or the symptoms of the disorder. The treatment may inhibit deterioration or worsening of a symptom of a disorder.

Epothilone B is also referred to herein as patupilone and EP0906.

Ixabepilone is also referred to herein as Ixempra™.

Epothilone D is also referred to herein as KOS-862.

Dehydelone is also referred to herein as KOS-1584.

ZK-EPO is also referred to herein as sagopilone.

The term “acyl” refers to an alkylcarbonyl, cycloalkylcarbonyl, arylcarbonyl, heterocyclylcarbonyl, or heteroarylcarbonyl substituent, any of which may be further substituted (e.g., by one or more substituents). Exemplary acyl groups include acetyl group (CH₃C(O)—), benzoyl (C₆H₅C(O)—), and acetylamino acids (e.g., acetylglycine, CH₃C(O)NHCH₂C(O)—.

The term “alkenyl” refers to an aliphatic group containing at least one double bond.

The terms “alkoxyl” or “alkoxy” refers to an alkyl group, as defined below, having an oxygen radical attached thereto. Representative alkoxyl groups include methoxy, ethoxy, propyloxy, tert-butoxy and the like. An “ether” is two hydrocarbons covalently linked by an oxygen.

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁-C₃₀ for straight chains, C₃-C₃₀ for branched chains), and more preferably 20 or fewer, and most preferably 10 or fewer. Likewise, preferred cycloalkyls have from 3-10 carbon atoms in their ring structure, and more preferably have 5, 6 or 7 carbons in the ring structure. The term “alkylenyl” refers to a divalent alkyl, e.g., —CH₂—, —CH₂CH₂—, and —CH₂CH₂CH₂—.

The term “alkynyl” refers to an aliphatic group containing at least one triple bond.

The term “aralkyl” or “arylalkyl” refers to an alkyl group substituted with an aryl group (e.g., a phenyl or naphthyl).

The term “aryl” includes 5-14 membered single-ring or bicyclic aromatic groups, for example, benzene, naphthalene, and the like. The aromatic ring can be substituted at one or more ring positions with such substituents as described above, for example, halogen, azide, alkyl, aralkyl, alkenyl, alkynyl, cycloalkyl, polycyclyl, hydroxyl, alkoxyl, amino, nitro, sulfhydryl, imino, amido, phosphate, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocyclyls. Each ring can contain, e.g., 5-7 members. The term “arylene” refers to a divalent aryl, as defined herein.

The term “arylalkenyl” refers to an alkenyl group substituted with an aryl group.

The terms “halo” and “halogen” means halogen and includes chloro, fluoro, bromo, and iodo.

The terms “hetaralkyl”, “heteroaralkyl” or “heteroarylalkyl” refers to an alkyl group substituted with a heteroaryl group.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, 3, or 4 atoms of each ring may be substituted by a substituent. Examples of heteroaryl groups include pyridyl, furyl or furanyl, imidazolyl, benzimidazolyl, pyrimidinyl, thiophenyl or thienyl, quinolinyl, indolyl, thiazolyl, and the like. The term “heteroarylene” refers to a divalent heteroaryl, as defined herein.

The term “heteroarylalkenyl” refers to an alkenyl group substituted with a heteroaryl group.

The term “substituents” refers to a group “substituted” on an alkyl, cycloalkyl, alkenyl, alkynyl, heterocyclyl, heterocycloalkenyl, cycloalkenyl, aryl, or heteroaryl group at any atom of that group. Any atom can be substituted. Suitable substituents include, without limitation, alkyl (e.g., C1, C2, C3, C4, C5, C6, C7, C8, C9, C10, C11, C12 straight or branched chain alkyl), cycloalkyl, haloalkyl (e.g., perfluoroalkyl such as CF₃), aryl, heteroaryl, aralkyl, heteroaralkyl, heterocyclyl, alkenyl, alkynyl, cycloalkenyl, heterocycloalkenyl, alkoxy, haloalkoxy (e.g., perfluoroalkoxy such as OCF₃), halo, hydroxy, carboxy, carboxylate, cyano, nitro, amino, alkyl amino, SO₃H, sulfate, phosphate, methylenedioxy (—O—CH₂—O— wherein oxygens are attached to vicinal atoms), ethylenedioxy, oxo, thioxo (e.g., C═S), imino (alkyl, aryl, aralkyl), S(O)ₙalkyl (where n is 0-2), S(O)ₙ aryl (where n is 0-2), S(O)ₙ heteroaryl (where n is 0-2), S(O)ₙ heterocyclyl (where n is 0-2), amine (mono-, di-, alkyl, cycloalkyl, aralkyl, heteroaralkyl, aryl, heteroaryl, and combinations thereof), ester (alkyl, aralkyl, heteroaralkyl, aryl, heteroaryl), amide (mono-, di-, alkyl, aralkyl, heteroaralkyl, aryl, heteroaryl, and combinations thereof), sulfonamide (mono-, di-, alkyl, aralkyl, heteroaralkyl, and combinations thereof). In one aspect, the substituents on a group are independently any one single, or any subset of the aforementioned substituents. In another aspect, a substituent may itself be substituted with any one of the above substituents.

### Polymer-Agent Conjugates

A polymer-agent conjugate described herein includes a polymer (e.g., a hydrophobic polymer or a polymer containing a hydrophilic portion and a hydrophobic portion) and an agent (e.g., an epothilone). An agent described herein may be attached to a polymer described herein, e.g., directly or through a linker. An agent may be attached to a hydrophobic polymer (e.g., PLGA), or a polymer having a hydrophobic portion and a hydrophilic portion (e.g., PEG-PLGA). An agent may be attached to a terminal end of a polymer, to both terminal ends of a polymer, or to a point along a polymer chain. In some embodiments, multiple agents may be attached to points along a polymer chain. In some embodiments, multiple agents may be attached to points along a polymer chain, or multiple agents may be attached to a terminal end of a polymer via a multifunctional linker.

Polymers

A wide variety of polymers and methods for forming polymer-agent conjugates and particles therefrom are known in the art of drug delivery. Any polymer may be used in accordance with the present invention. Polymers may be natural or unnatural (synthetic) polymers. Polymers may be homopolymers or copolymers containing two or more monomers. Polymers may be linear or branched.

If more than one type of repeat unit is present within the polymer, then the polymer is said to be a “copolymer.” It is to be understood that in any embodiment employing a polymer, the polymer being employed may be a copolymer. The repeat units forming the copolymer may be arranged in any fashion. For example, the repeat units may be arranged in a random order, in an alternating order, or as a “block” copolymer, i.e., containing one or more regions each containing a first repeat unit (e.g., a first block), and one or more regions each containing a second repeat unit (e.g., a second block), etc. Block copolymers may have two (a diblock copolymer), three (a triblock copolymer), or more numbers of distinct blocks. In terms of sequence, copolymers may be random, block, or contain a combination of random and block sequences.

Hydrophobic Polymers

A polymer-agent conjugate or particle described herein may include a hydrophobic polymer. The hydrophobic polymer may be attached to an agent. Exemplary hydrophobic polymers include the following: acrylates including methyl acrylate, ethyl acrylate, propyl acrylate, n-butyl acrylate (BA), isobutyl acrylate, 2-ethyl acrylate, and t-butyl acrylate; methacrylates including ethyl methacrylate, n-butyl methacrylate, and isobutyl methacrylate; acrylonitriles; methacrylonitrile; vinyls including vinyl acetate, vinylversatate, vinylpropionate, vinylformamide, vinylacetamide, vinylpyridines, and vinylimidazole; aminoalkyls including aminoalkylacrylates, aminoalkylmethacrylates, and aminoalkyl(meth)acrylamides; styrenes; cellulose acetate phthalate; cellulose acetate succinate; hydroxypropylmethylcellulose phthalate; poly(D,L-lactide); poly(D,L-lactide-co-glycolide); poly(glycolide); poly(hydroxybutyrate); poly(alkylcarbonate); poly(orthoesters); polyesters; poly(hydroxyvaleric acid); polydioxanone; poly(ethylene terephthalate); poly(malic acid); poly(tartronic acid); polyanhydrides; polyphosphazenes; poly(amino acids) and their copolymers (see generally, Svenson, S (ed.)., Polymeric Drug Delivery: Volume I: Particulate Drug Carriers. 2006; ACS Symposium Series; Amiji, M. M (ed.)., Nanotechnology for Cancer Therapy. 2007; Taylor & Francis Group, LLP; Nair et al. Prog. Polym. Sci. (2007) 32: 762-798); hydrophobic peptide-based polymers and copolymers based on poly(L-amino acids) (Lavasanifar, A., et al., Advanced Drug Delivery Reviews (2002) 54:169-190); poly(ethylene-vinyl acetate) (“EVA”) copolymers; silicone rubber; polyethylene; polypropylene; polydienes (polybutadiene, polyisoprene and hydrogenated forms of these polymers); maleic anhydride copolymers of vinyl methylether and other vinyl ethers; polyamides (nylon 6,6); polyurethane; poly(ester urethanes); poly(ether urethanes); and poly(ester-urea).

Hydrophobic polymers useful in preparing the polymer-agent conjugates or particles described herein also include biodegradable polymers. Examples of biodegradable polymers include polylactides, polyglycolides, caprolactone-based polymers, poly(caprolactone), polydioxanone, polyanhydrides, polyamines, polyesteramides, polyorthoesters, polydioxanones, polyacetals, polyketals, polycarbonates, polyphosphoesters, polyesters, polybutylene terephthalate, polyorthocarbonates, polyphosphazenes, succinates, poly(malic acid), poly(amino acids), poly(vinylpyrrolidone), polyethylene glycol, polyhydroxycellulose, polysaccharides, chitin, chitosan and hyaluronic acid, and copolymers, terpolymers and mixtures thereof. Biodegradable polymers also include copolymers, including caprolactone-based polymers, polycaprolactones and copolymers that include polybutylene terephthalate.

In some embodiments, the polymer is a polyester synthesized from monomers selected from the group consisting of D,L-lactide, D-lactide, L-lactide, D,L-lactic acid, D-lactic acid, L-lactic acid, glycolide, glycolic acid, ε-caprolactone, ε-hydroxy hexanoic acid, γ-butyrolactone, γ-hydroxy butyric acid, δ-valerolactone, δ-hydroxy valeric acid, hydroxybutyric acids, and malic acid.

A copolymer may also be used in a polymer-agent conjugate or particle described herein. In some embodiments, a polymer may be PLGA, which is a biodegradable random copolymer of lactic acid and glycolic acid. A PLGA polymer may have varying ratios of lactic acid:glycolic acid, e.g., ranging from about 0.1:99.9 to about 99.9:0.1 (e.g., from about 75:25 to about 25:75, from about 60:40 to 40:60, or about 55:45 to 45:55). In some embodiments, e.g., in PLGA, the ratio of lactic acid monomers to glycolic acid monomers is 50:50, 60:40 or 75:25.

In particular embodiments, by optimizing the ratio of lactic acid to glycolic acid monomers in the PLGA polymer of the polymer-agent conjugate or particle, parameters such as water uptake, agent release (e.g., “controlled release”) and polymer degradation kinetics may be optimized. Furthermore, tuning the ratio will also affect the hydrophobicity of the copolymer, which may in turn affect drug loading.

In certain embodiments wherein the biodegradable polymer also has an agent or other material attached to it, the biodegradation rate of such polymer may be characterized by a release rate of such materials. In such circumstances, the biodegradation rate may depend on not only the chemical identity and physical characteristics of the polymer, but also on the identity of material(s) attached thereto. Degradation of the subject compositions includes not only the cleavage of intramolecular bonds, e.g., by oxidation and/or hydrolysis, but also the disruption of intermolecular bonds, such as dissociation of host/guest complexes by competitive complex formation with foreign inclusion hosts. In some embodiments, the release can be affected by an additional component in the particle, e.g., a compound having at least one acidic moiety (e.g., free-acid PLGA).

In certain embodiments, polymeric formulations of the present invention biodegrade within a period that is acceptable in the desired application. In certain embodiments, such as in vivo therapy, such degradation occurs in a period usually less than about five years, one year, six months, three months, one month, fifteen days, five days, three days, or even one day on exposure to a physiological solution with a pH between 4 and 8 having a temperature of between 25° C. and 37° C. In other embodiments, the polymer degrades in a period of between about one hour and several weeks, depending on the desired application.

When polymers are used for delivery of pharmacologically active agents in vivo, it is important that the polymers themselves be nontoxic and that they degrade into non-toxic degradation products as the polymer is eroded by the body fluids. Many synthetic biodegradable polymers, however, yield oligomers and monomers upon erosion in vivo that adversely interact with the surrounding tissue (D. F. Williams, J. Mater. Sci. 1233 (1982)). To minimize the toxicity of the intact polymer carrier and its degradation products, polymers have been designed based on naturally occurring metabolites. Exemplary polymers include polyesters derived from lactic and/or glycolic acid and polyamides derived from amino acids.

A number of biodegradable polymers are known and used for controlled release of pharmaceuticals. Such polymers are described in, for example, U.S. Pat. Nos. 4,291,013; 4,347,234; 4,525,495; 4,570,629; 4,572,832; 4,587,268; 4,638,045; 4,675,381; 4,745,160; and 5,219,980; and PCT publication WO2006/014626, each of which is hereby incorporated by reference in its entirety.

A hydrophobic polymer described herein may have a variety of end groups. In some embodiments, the end group of the polymer is not further modified, e.g., when the end group is a carboxylic acid, a hydroxy group or an amino group. In some embodiments, the end group may be further modified. For example, a polymer with a hydroxyl end group may be derivatized with an acyl group to yield an acyl-capped polymer (e.g., an acetyl-capped polymer or a benzoyl capped polymer), an alkyl group to yield an alkoxy-capped polymer (e.g., a methoxy-capped polymer), or a benzyl group to yield a benzyl-capped polymer.

A hydrophobic polymer may have a weight average molecular weight ranging from about 1 kDa to about 20 kDa (e.g., from about 1 kDa to about 15 kDa, from about 2 kDa to about 12 kDa, from about 6 kDa to about 20 kDa, from about 5 kDa to about 15 kDa, from about 6 kDa to about 13 kDa, from about 7 kDa to about 11 kDa, from about 5 kDa to about 10 kDa, from about 7 kDa to about 10 kDa, from about 5 kDa to about 7 kDa, from about 6 kDa to about 8 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa or about 17 kDa).

A hydrophobic polymer described herein may have a polymer polydispersity index (PDI) of less than or equal to about 2.5 (e.g., less than or equal to about 2.2, or less than or equal to about 2.0). In some embodiments, a hydrophobic polymer described herein may have a polymer PDI of about 1.0 to about 2.5, about 1.0 to about 2.0, about 1.0 to about 1.7, or from about 1.0 to about 1.6.

A particle described herein may include varying amounts of a hydrophobic polymer, e.g., from about 20% to about 90% by weight (e.g., from about 20% to about 80%, from about 25% to about 75%, or from about 30% to about 70%).

A hydrophobic polymer described herein may be commercially available, e.g., from a commercial supplier such as BASF, Boehringer Ingelheim, Durcet Corporation, Purac America and SurModics Pharmaceuticals. A polymer described herein may also be synthesized. Methods of synthesizing polymers are known in the art (see, for example, Polymer Synthesis: Theory and Practice Fundamentals, Methods, Experiments. D. Braun et al., 4th edition, Springer, Berlin, 2005). Such methods include, for example, polycondensation, radical polymerization, ionic polymerization (e.g., cationic or anionic polymerization), or ring-opening metathesis polymerization.

A commercially available or synthesized polymer sample may be further purified prior to formation of a polymer-agent conjugate or incorporation into a particle or composition described herein. In some embodiments, purification may reduce the polydispersity of the polymer sample. A polymer may be purified by precipitation from solution, or precipitation onto a solid such as Celite. A polymer may also be further purified by size exclusion chromatography (SEC).

Polymers Containing a Hydrophilic Portion and a Hydrophobic Portion

A polymer-agent conjugate or particle described herein may include a polymer containing a hydrophilic portion and a hydrophobic portion. A polymer containing a hydrophilic portion and a hydrophobic portion may be a copolymer of a hydrophilic block coupled with a hydrophobic block. These copolymers may have a weight average molecular weight between about 5 kDa and about 30 kDa (e.g., from about 5 kDa to about 25 kDa, from about 10 kDa to about 22 kDa, from about 10 kDa to about 15 kDa, from about 12 kDa to about 22 kDa, from about 7 kDa to about 15 kDa, from about 15 kDa to about 19 kDa, or from about 11 kDa to about 13 kDa, e.g., about 9 kDa, about 10 kDa, about 11 kDa, about 12 kDa, about 13 kDa, about 14 kDa about 15 kDa, about 16 kDa, about 17 kDa, about 18 kDa or about 19 kDa). The polymer containing a hydrophilic portion and a hydrophobic portion may be attached to an agent.

Examples of suitable hydrophobic portions of the polymers include those described above. The hydrophobic portion of the copolymer may have a weight average molecular weight of from about 1 kDa to about 20 kDa (e.g., from about 1 kDa to about 18 kDa, 17 kDa, 16 kDa, 15 kDa, 14 kDa or 13 kDa, from about 2 kDa to about 12 kDa, from about 6 kDa to about 20 kDa, from about 5 kDa to about 18 kDa, from about 7 kDa to about 17 kDa, from about 8 kDa to about 13 kDa, from about 9 kDa to about 11 kDa, from about 10 kDa to about 14 kDa, from about 6 kDa to about 8 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa or about 17 kDa).

Examples of suitable hydrophilic portions of the polymers include the following: carboxylic acids including acrylic acid, methacrylic acid, itaconic acid, and maleic acid; polyoxyethylenes or polyethylene oxide; polyacrylamides and copolymers thereof with dimethylaminoethylmethacrylate, diallyldimethylammonium chloride, vinylbenzylthrimethylammonium chloride, acrylic acid, methacrylic acid, 2-acrylamido-2-methylpropane sulfonic acid and styrene sulfonate, poly(vinylpyrrolidone), starches and starch derivatives, dextran and dextran derivatives; polypeptides, such as polylysines, polyarginines, polyglutamic acids; polyhyaluronic acids, alginic acids, polylactides, polyethyleneimines, polyionenes, polyacrylic acids, and polyiminocarboxylates, gelatin, and unsaturated ethylenic mono or dicarboxylic acids. A listing of suitable hydrophilic polymers can be found in Handbook of Water-Soluble Gums and Resins, R. Davidson, McGraw-Hill (1980).

The hydrophilic portion of the copolymer may have a weight average molecular weight of from about 1 kDa to about 21 kDa (e.g., from about 1 kDa to about 3 kDa, e.g., about 2 kDa, or from about 2 kDa to about 5 kDa, e.g., about 3.5 kDa, or from about 4 kDa to about 6 kDa, e.g., about 5 kDa).

A polymer containing a hydrophilic portion and a hydrophobic portion may be a block copolymer, e.g., a diblock or triblock copolymer. In some embodiments, the polymer may be a diblock copolymer containing a hydrophilic block and a hydrophobic block. In some embodiments, the polymer may be a triblock copolymer containing a hydrophobic block, a hydrophilic block and another hydrophobic block. The two hydrophobic blocks may be the same hydrophobic polymer or different hydrophobic polymers. The block copolymers used herein may have varying ratios of the hydrophilic portion to the hydrophobic portion, e.g., ranging from 1:1 to 1:40 by weight (e.g., about 1:1 to about 1:10 by weight, about 1:1 to about 1:2 by weight, or about 1:3 to about 1:6 by weight).

A polymer containing a hydrophilic portion and a hydrophobic portion may have a variety of end groups. In some embodiments, the end group may be a hydroxy group or an alkoxy group. In some embodiments, the end group of the polymer is not further modified. In some embodiments, the end group may be further modified. For example, the end group may be capped with an alkyl group, to yield an alkoxy-capped polymer (e.g., a methoxy-capped polymer), or may be derivatized with a targeting agent (e.g., folate) or a dye (e.g., rhodamine).

A polymer containing a hydrophilic portion and a hydrophobic portion may include a linker between the two blocks of the copolymer. Such a linker may be an amide, ester, ether, amino, carbamate or carbonate linkage, for example.

A polymer containing a hydrophilic portion and a hydrophobic portion described herein may have a polymer polydispersity index (PDI) of less than or equal to about 2.5 (e.g., less than or equal to about 2.2, or less than or equal to about 2.0, or less than or equal to about 1.5). In some embodiments, the polymer PDI is from about 1.0 to about 2.5, e.g., from about 1.0 to about 2.0, from about 1.0 to about 1.8, from about 1.0 to about 1.7, or from about 1.0 to about 1.6.

A particle described herein may include varying amounts of a polymer containing a hydrophilic portion and a hydrophobic portion, e.g., up to about 50% by weight (e.g., from about 4 to about 50%, about 5%, about 10%, about 15%, about 20%, about 25%, about 30%, about 35%, about 40%, about 45% or about 50% by weight). For example, the percent by weight of the second polymer within the particle is from about 3% to 30%, from about 5% to 25% or from about 8% to 23%.

A polymer containing a hydrophilic portion and a hydrophobic portion described herein may be commercially available, or may be synthesized. Methods of synthesizing polymers are known in the art (see, for example, Polymer Synthesis: Theory and Practice Fundamentals, Methods, Experiments. D. Braun et al., 4th edition, Springer, Berlin, 2005). Such methods include, for example, polycondensation, radical polymerization, ionic polymerization (e.g., cationic or anionic polymerization), or ring-opening metathesis polymerization. A block copolymer may be prepared by synthesizing the two polymer units separately and then conjugating the two portions using established methods. For example, the blocks may be linked using a coupling agent such as EDC (1-ethyl-3-(3-dimethylaminopropyl) carbodiimide hydrochloride). Following conjugation, the two blocks may be linked via an amide, ester, ether, amino, carbamate or carbonate linkage.

A commercially available or synthesized polymer sample may be further purified prior to formation of a polymer-agent conjugate or incorporation into a particle or composition described herein. In some embodiments, purification may remove lower molecular weight polymers that may lead to unfilterable polymer samples. A polymer may be purified by precipitation from solution, or precipitation onto a solid such as Celite. A polymer may also be further purified by size exclusion chromatography (SEC).

Agents

An agent to be delivered using a polymer-agent conjugate, particle or composition described herein may be an epothilone or an anti-cancer agent.

Epothilones

The term “epothilone,” as used herein, refers to any naturally occurring, synthetic, or semi-synthetic epothilone structure, for example, known in the art. The term epothilone also includes structures falling within the generic formulae X, XI, XII, XIII, XIV, XV, and XVI as provided herein.

Exemplary epothilones include those described generically and specifically herein. In some embodiments, the epothilone is epothilone B, ixabepilone, BMS310705, epothilone D, dehydelone, or sagopilone. The structures of all of these epothilones are provided below:

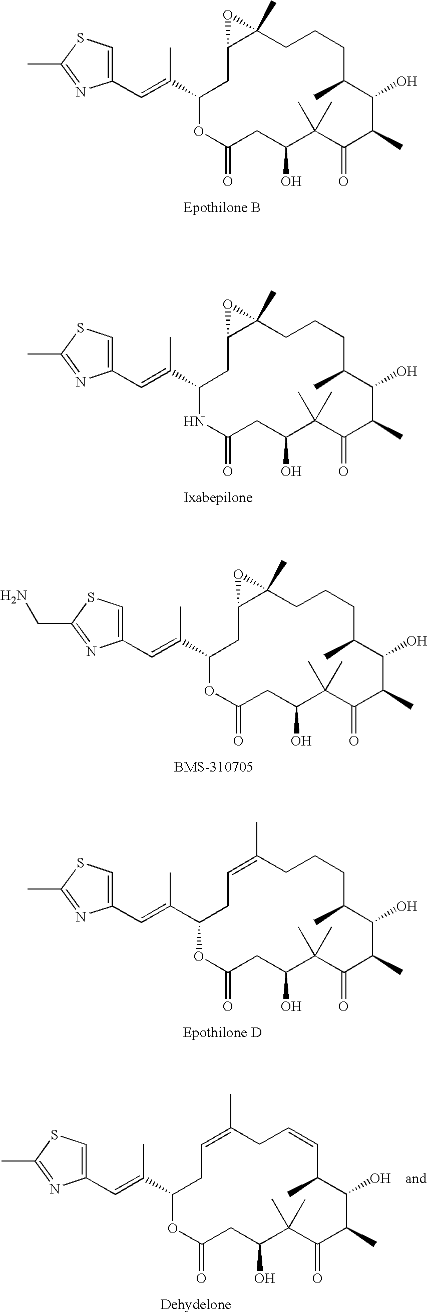

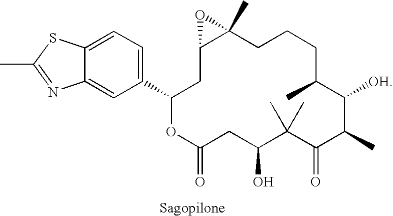

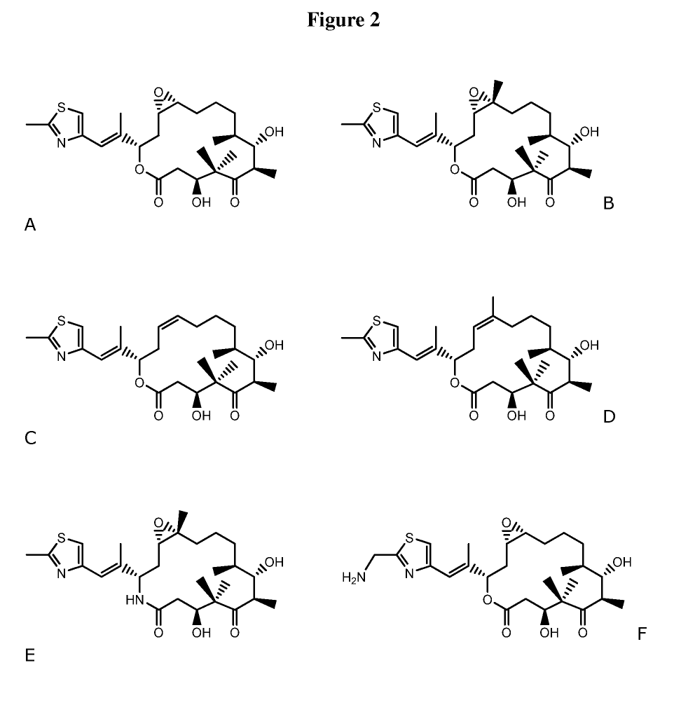

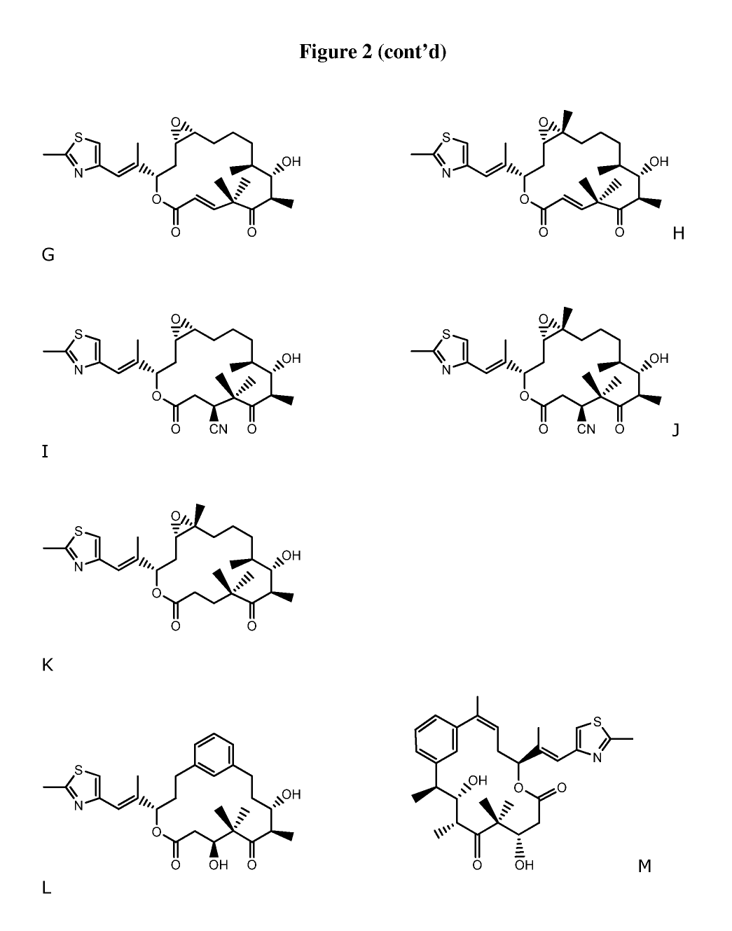

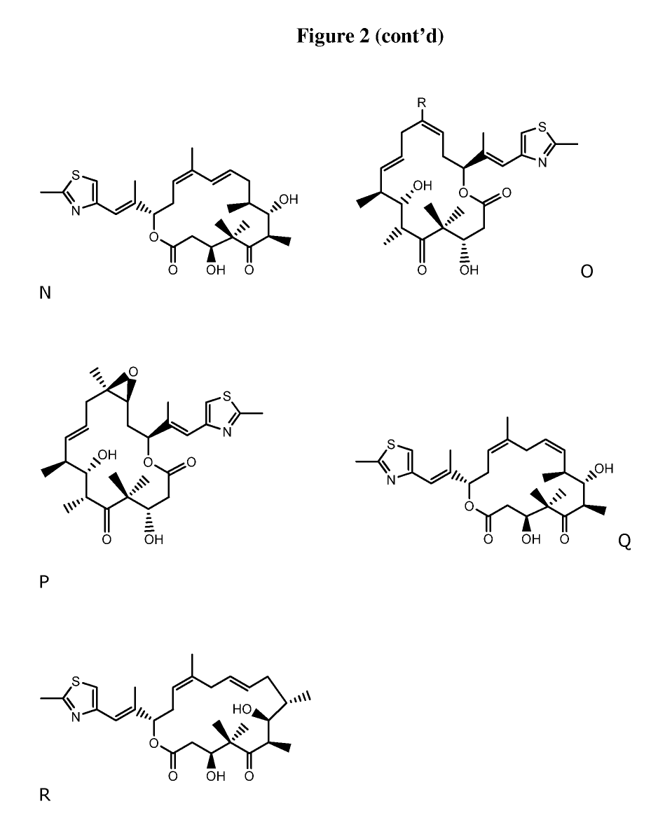

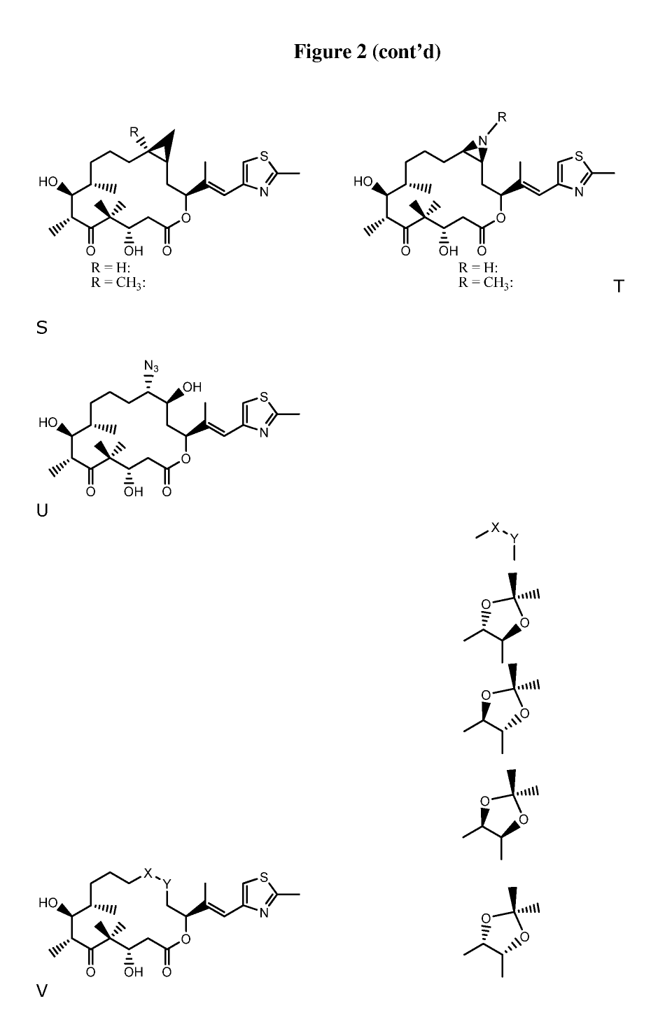

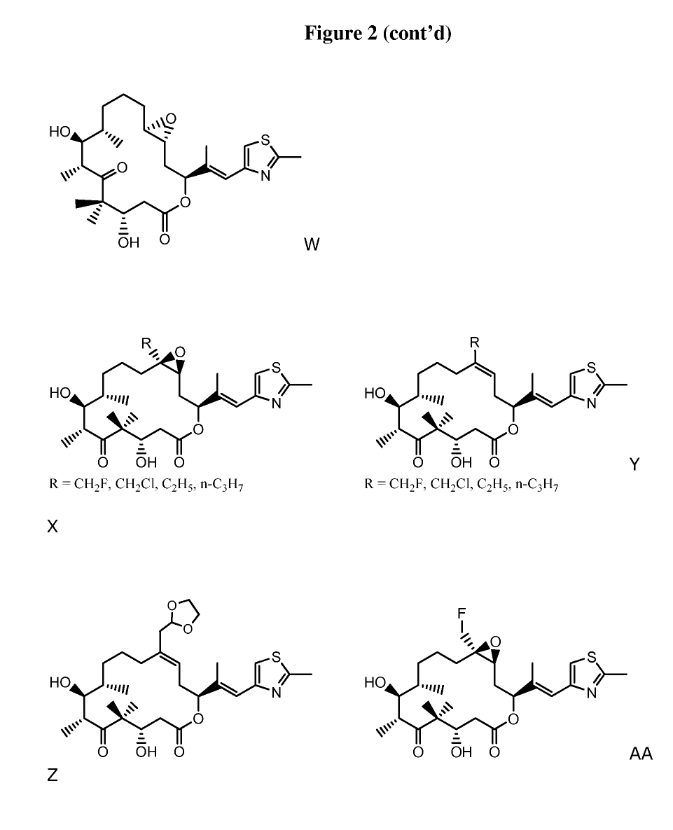

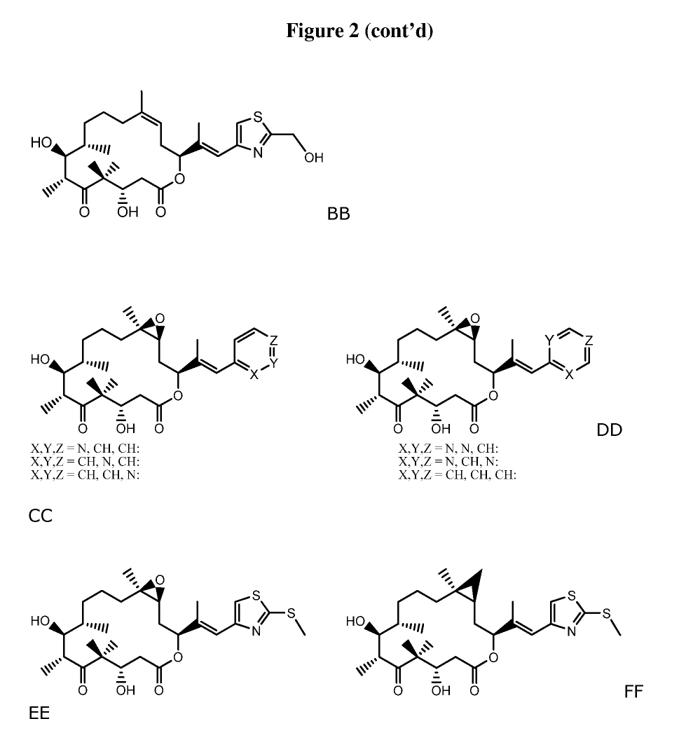

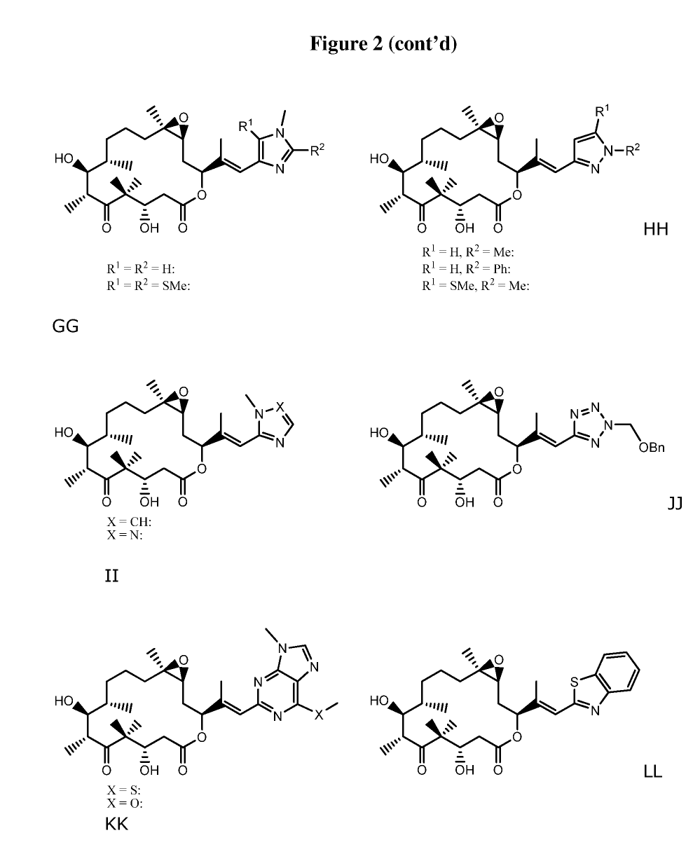

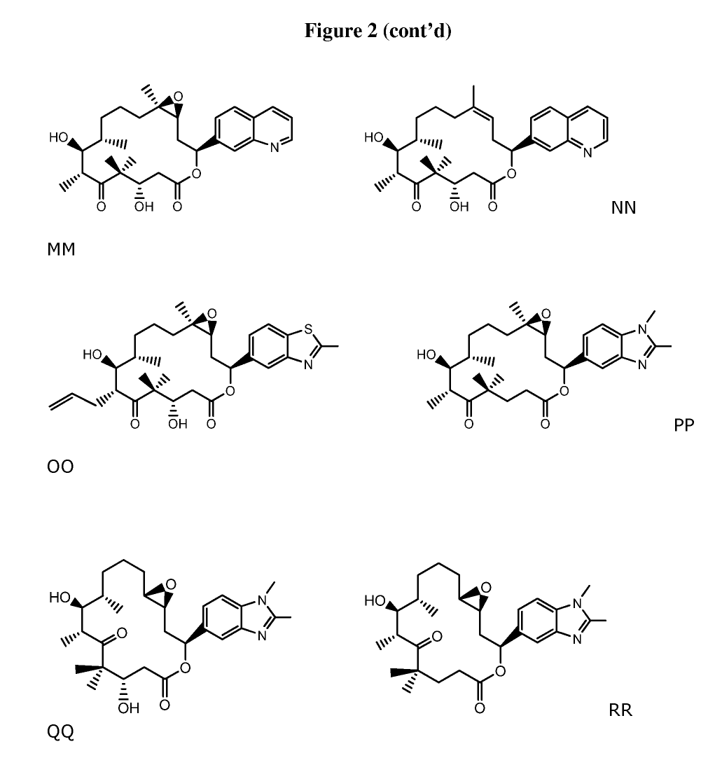

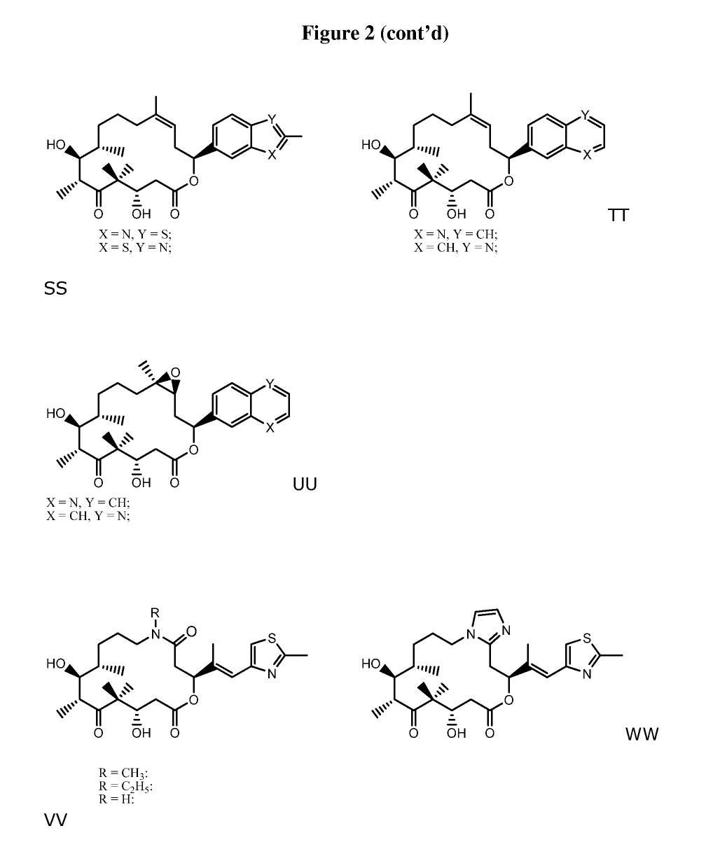

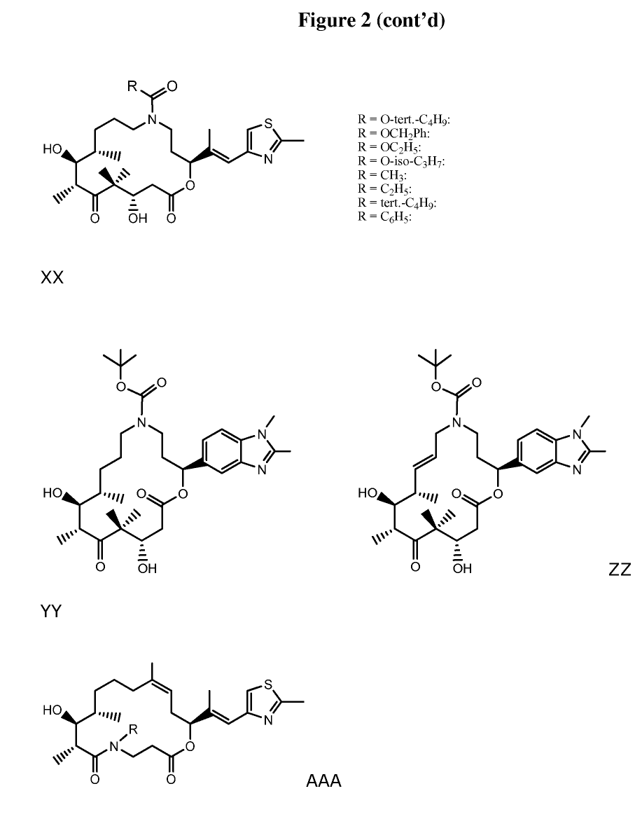

Other exemplary epothilones are also provided in FIG. 2 and disclosed in Altmann et al. “Epothilones as Lead Structures for New Anticancer Drugs-Pharmacology, Fermentation, and Structure-activity-relationships;” Progress in Drug Research (2008) Vol. 66, page 274-334, which is incorporated herein by reference.

Additionally, epothilones may be found, for example, in U.S. Pat. No. 7,317,100; U.S. Pat. No. 6,946,561; U.S. Pat. No. 6,350,878; U.S. Pat. No. 6,302,838; U.S. Pat. No. 7,030,147; U.S. Pat. No. 6,387,927; U.S. Pat. No. 6,346,404; US 2004/0038324; US 2009/0041715; US 2007/0129411; US 2005/0271669; US 2008/0139587; US 2004/0235796; US 2005/0282873; US 2006/0089327; WO 2008/071404; WO 2008/019820; WO 2007/121088; WO 1998/08849; EP 1198225; EP 1420780; EP 1385522; EP 1539768; EP 1485090; and EP 1463504, the contents of these references are incorporated herein in their entireties.

Further epothilones may be found, for example, in U.S. Pat. No. 6,410,301; U.S. Pat. No. 7,091,193; U.S. Pat. No. 7,402,421; U.S. Pat. No. 7,067,286; U.S. Pat. No. 6,489,314; U.S. Pat. No. 6,589,968; U.S. Pat. No. 6,893,859; U.S. Pat. No. 7,176,235; U.S. Pat. No. 7,220,560; U.S. Pat. No. 6,280,999; U.S. Pat. No. 7,070,964; US 2005/0148543; US 2005/0215604; US 2003/0134883; US 2008/0319211; US 2005/0277682; US 2005/0020558; US 2005/0203174; US 20020045609, US 2004/0167097; US 2004/0072882; US 2002/0137152; WO 2009/064800; and WO 2002/012534, the contents of these references are incorporated herein in their entireties.

Further epothilones may be found, for example, in U.S. Pat. No. 6,537,988; U.S. Pat. No. 7,312,237; U.S. Pat. No. 7,022,330; U.S. Pat. No. 6,670,384; U.S. Pat. No. 6,605,599; U.S. Pat. No. 7,125,899; U.S. Pat. No. 6,399,638; U.S. Pat. No. 7,053,069; U.S. Pat. No. 6,936,628; U.S. Pat. No. 7,211,593; U.S. Pat. No. 6,686,380; U.S. Pat. No. 6,727,276; U.S. Pat. No. 6,291,684; U.S. Pat. No. 6,780,620; U.S. Pat. No. 6,719,540; US 2009/0004277; US 2007/0276018; WO 2004/078978; and EP 1157023, the contents of these references are incorporated herein in their entireties.

Further epothilones may be found, for example, in US 2008/0146626; US 2009/0076098; WO 2009/003706 and WO 2009/074274, the contents of these references are incorporated herein in their entireties.

Further epothilones may be found, for example, in U.S. Pat. No. 7,169,930; U.S. Pat. No. 6,294,374; U.S. Pat. No. 6,380,394; and U.S. Pat. No. 6,441,186, the contents of these references are incorporated herein in their entireties.

Further epothilones may be found, for example, in U.S. Pat. No. 7,119,071; and German Application Serials Nos. DE 197 13 970.1, DE 100 51 136.8, DE 101 34 172.5, and DE 102 32 094.2, the contents of these references are incorporated herein in their entireties.

In some embodiments, the epothilone is attached to a targeting moiety such as a folate moiety. In some embodiments, the targeting moiety (e.g., a folate) is attached to a functional group on the epothilone such as a hydroxyl group or an amino group where appropriate. In some embodiments, the folate is attached to the epothilone directly. In some embodiments, the folate is attached to the epothilone via a linker. Epofolate (BMS-753493) is an example an epothilone attached to a folate, see, for example, U.S. Pat. No. 7,033,594, which is incorporated herein by reference.

In one embodiment, the epothilone is a compound of formula (X)

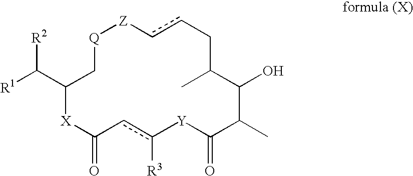

wherein

R¹ is aryl, heteroaryl, arylalkenyl or heteroarylalkenyl; each of which is optionally substituted with 1-3 R⁸;

R² is H or alkyl (e.g., a methyl); or

R¹ and R², when taken together with the carbon to which they are attached, form an aryl or a heteroaryl moiety optionally substituted with 1-3 R⁸;

R³ is H, OH, NH₂, or CN;

X is O or NR⁴;

R⁴ is H, alkyl, —C(O)Oalkyl, —C(O)Oarylalkyl, —C(O)NR⁵alkyl, —C(O)NR⁵arylalkyl, —C(O)alkyl, —C(O)aryl or arylalkyl;

Y is CR⁵R⁶, O or NR⁷;

each of R⁵ and R⁶ is independently H or alkyl (e.g., methyl);

R⁷ is H, alkyl, —C(O)Oalkyl, —C(O)Oarylalkyl, —C(O)NR⁵alkyl, —C(O) NR⁵arylalkyl, —C(O)alkyl, —C(O)aryl or arylalkyl;

each R⁸, for each occurrence, is independently alkyl, aminoalkyl, hydroxyalkyl, alkylthiol, aryl, arylalkyloxyalkyl or alkoxy;

Q-Z, when taken together, form

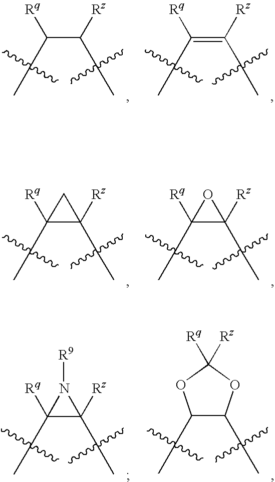

heteroarylenyl, C(O)NR⁴, NR⁴C(O), CR⁵R⁶NR⁴, or NR⁴CR⁵R⁶;

R^{q} is H, alkyl (e.g., methyl) or hydroxy;

R^{z} is H, alkyl (e.g., methyl), haloalkyl (e.g., CF₃), heterocyclylalkyl or N₃;

R⁹ is H, alkyl, —C(O)Oalkyl, —C(O)Oarylalkyl, —C(O)NR⁵alkyl, —C(O) NR⁵arylalkyl, —C(O)alkyl, —C(O)aryl or arylalkyl; and

each [symbol] for each occurrence, is independently a single or double bond.

In some embodiments, R¹ is

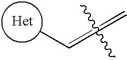

optionally substituted with 1-3 R⁸.

In some embodiments, HET is a five membered ring heteroaryl optionally substituted with 1-3 R⁸.

In some embodiments, HET is a thiazolyl optionally substituted with 1-3 R⁸. In some embodiments, HET is substituted with alkyl (e.g., methyl), aminoalkyl (e.g., aminomethyl), alkylthiol (e.g., methylthiol), hydroxyalkyl (e.g., hydroxymethyl), alkoxy (e.g., methoxy) or aryl (e.g., phenyl).

In some embodiments, HET is substituted with alkyl (e.g., methyl) or amino alkyl.

In some embodiments, HET is

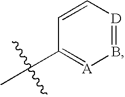

wherein each of A, B and D is independently CH or N. In some embodiments, A is N, B is CH and D is CH. In some embodiments, A is CH, B is N and D is CH. In some embodiments, A is CH, B is CH and D is N.

In some embodiments, HET is

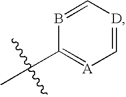

wherein each of A, B and D is independently CH or N. In some embodiments, A is N, B is N and D is CH. In some embodiments, A is N, B is CH and D is N. In some embodiments, A is CH, B is CH and D is CH.

In some embodiments, HET is

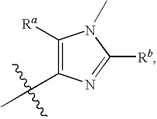

wherein each R^{a} and R^{b} is independently H or —SMe.

In some embodiments, HET is

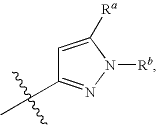

wherein each R^{a} is H, alkyl or —Salkyl; and R^{b} is H, alkyl (e.g., methyl) or aryl (e.g., phenyl).

In some embodiments, HET is

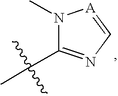

wherein A is CH or N.

In some embodiments, HET is

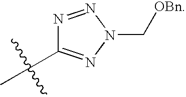

In some embodiments, HET is

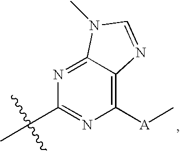

wherein A is S or O.

In some embodiments, HET is

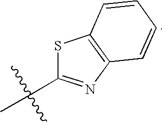

In some embodiments R² is H.

In some embodiments, R² is alkyl (e.g., methyl).

In some embodiments, R¹ and R², when taken together with the carbon to which they are attached, form an aryl or a heteroaryl moiety optionally substituted with 1-3 R⁸.

In some embodiments, R¹ and R², when taken together with the carbon to which they are attached, form a heteroaryl moiety optionally substituted with 1-3 R⁸.

In some embodiments, the heteroaryl moiety is a bicyclic heteroaryl moiety.

In some embodiments, R¹ and R², when taken together with the carbon to which they are attached, are

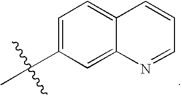

In some embodiments, R¹ and R², when taken together with the carbon to which they are attached, are

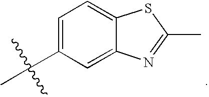

In some embodiments, R¹ and R², when taken together with the carbon to which they are attached, are

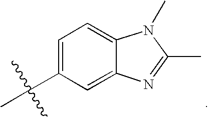

In some embodiments, R¹ and R², when taken together with the carbon to which they are attached, are

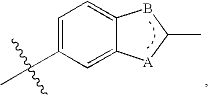

wherein A is N and B is S or wherein A is S and B is N.

In some embodiments, R¹ and R², when taken together with the carbon to which they are attached, are

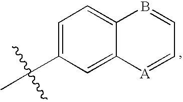

wherein A is N and B is CH or wherein A is CH and B is N.

In some embodiments,

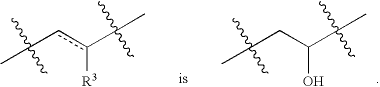

In some embodiments,

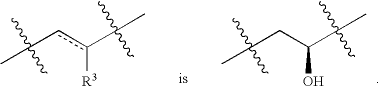

In some embodiments,

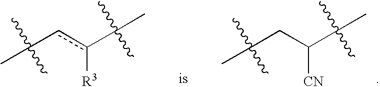

In some embodiments,

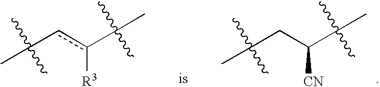

In some embodiments,

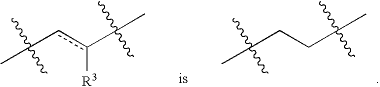

In some embodiments,

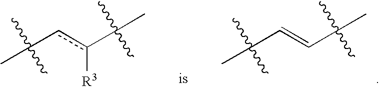

In some embodiments, X is O.

In some embodiments, X is NR⁴ (e.g., NH).

In some embodiments, Y is CR⁵R⁶. In some embodiments, Y is

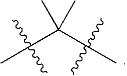

In some embodiments, Y is CH₂.

In some embodiments, Y is NR⁷ (e.g., NH or NMe).

In some embodiments, Q-Z, when taken together, form

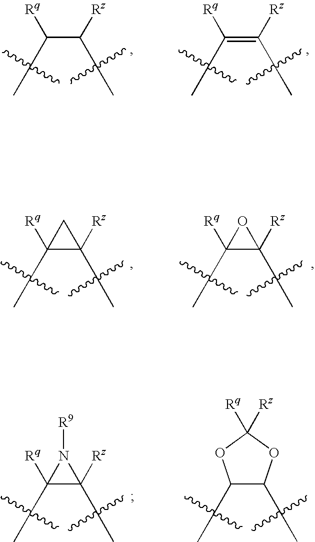

or heteroarylenyl.

In some embodiments, Q-Z, when taken together, form

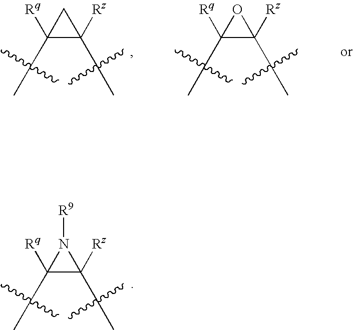

In some embodiments, Q-Z, when taken together, form

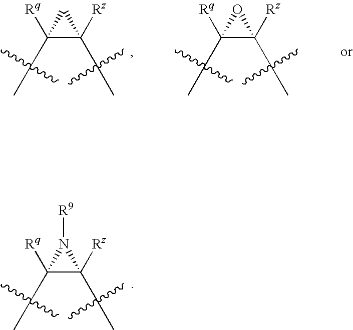

In some embodiments, Q-Z, when taken together, form

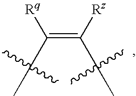

wherein R^{q} is H and R^{z} is H or alkyl (e.g., methyl).

In some embodiments, Q-Z, when taken together, form

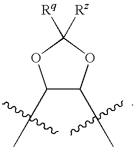

In some embodiments, both R^{q} and R^{z} are methyl. In some embodiments,

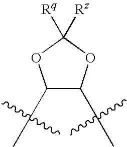

is selected from

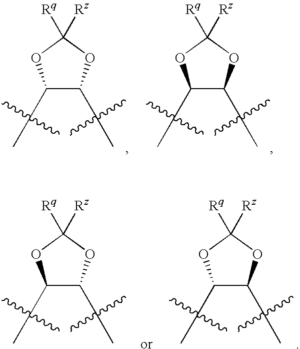

In some embodiments, both R^{q} and R^{z} are methyl.

In some embodiments, Q-Z, when taken together, form a heteroarylenyl. In some embodiments, Q-Z, when taken together, form

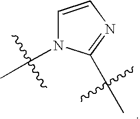

In some embodiments, Q-Z, when taken together, form C(O)NR⁴. In some embodiments, R⁴ is H or alkyl (e.g., methyl or ethyl).

In some embodiments, Q-Z, when taken together, form NR⁴C(O). In some embodiments, R⁴ is H or alkyl (e.g., methyl or ethyl).

In some embodiments, Q-Z, when taken together, form CH₂NR⁴. In some embodiments, R⁴ is H, alkyl, —C(O)Oalkyl, —C(O)Oarylalkyl, —C(O)alkyl, —C(O)aryl or arylalkyl. In some embodiments, R⁴ is —C(O)Oalkyl, —C(O)Oarylalkyl, —C(O)alkyl, —C(O)aryl or arylalkyl.

In some embodiments, Q-Z, when taken together, form NR⁴CH₂. In some embodiments, R⁴ is H, alkyl, —C(O)Oalkyl, —C(O)Oarylalkyl, —C(O)alkyl, —C(O)aryl or arylalkyl. In some embodiments, R⁴ is —C(O)Oalkyl, —C(O)Oarylalkyl, —C(O)alkyl, —C(O)aryl or arylalkyl.

In some embodiments, the compound of formula (X) is a compound of formula (Xa)

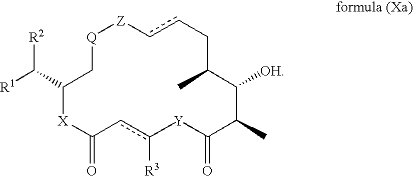

In some embodiments, the compound of formula (X) is a compound of formula (Xb)

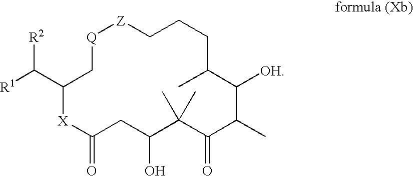

In some embodiments, the compound of formula (X) is a compound of formula (Xc)

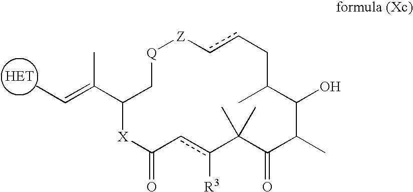

wherein HET is an optionally substituted heteroaryl.

In some embodiments, HET is an optionally substituted 5 membered ring.

In some embodiments, the compound of formula (X) is a compound of formula (Xd)

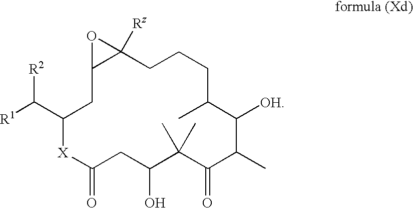

In some embodiments, the compound of formula (X) is a compound of formula (Xe)

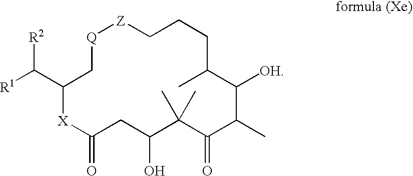

In some embodiments, the compound of formula (X) is a compound of formula (Xf)

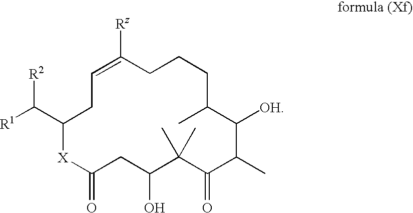

In some embodiments, the compound of formula (X) is a compound of formula (Xg)

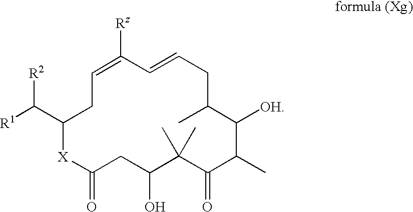

In one embodiment, the epothilone is a compound of formula (XI)

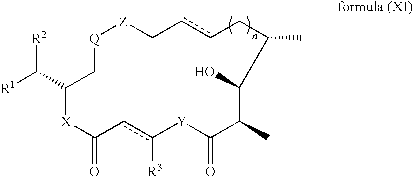

wherein

R¹ is aryl, heteroaryl, arylalkenyl, or heteroarylalkenyl; each of which is optionally substituted with 1-3 R⁸;

R² is H or alkyl (e.g., methyl); or

R¹ and R², when taken together with the carbon to which they are attached, form an aryl or a heteroaryl moiety optionally substituted with 1-3 R⁸;

R³ is H, OH, NH₂ or CN;

X is O or NR⁴;

R⁴ is H, alkyl, —C(O)Oalkyl, —C(O)Oarylalkyl, —C(O)NR⁵alkyl, —C(O) NR⁵arylalkyl, —C(O)alkyl, —C(O)aryl or arylalkyl;

Y is CR⁵R⁶, O or NR⁷;

each of R⁵ and R⁶ is independently H or alkyl (e.g., methyl);

R⁷ is H, alkyl, —C(O)Oalkyl, —C(O)Oarylalkyl, —C(O)NR⁵alkyl, —C(O) NR⁵arylalkyl, —C(O)alkyl, —C(O)aryl or arylalkyl;

each R⁸, for each occurrence, is independently alkyl, aminoalkyl, hydroxyalkyl, alkylthiol, aryl, arylalkyloxyalkyl or alkoxy;

Q-Z, when taken together, form

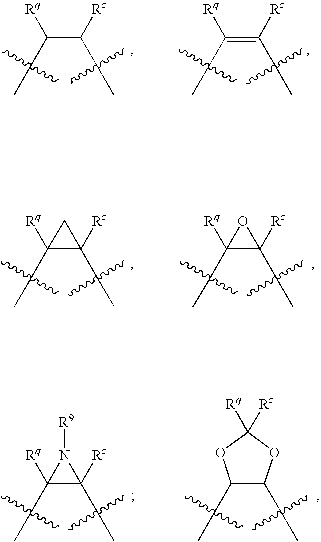

heteroarylenyl, C(O)NR⁴, NR⁴C(O), CR⁵R⁶NR⁴, or NR⁴CR⁵R⁶NR⁴;

R^{q} is H, alkyl (e.g., methyl) or hydroxy;

R^{z} is H, alkyl (e.g., methyl), haloalkyl (e.g., CF₃), heterocyclylalkyl or N₃;

R⁹ is H, alkyl, —C(O)Oalkyl, —C(O)Oarylalkyl, —C(O)NR⁵alkyl, —C(O) NR⁵arylalkyl, —C(O)alkyl, —C(O)aryl or arylalkyl;

each [symbol] for each occurrence, is independently a single or double bond; and

n is 0, 1 or 2.

In some embodiments, R¹ is

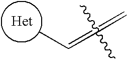

optionally substituted with 1-3 R⁸. In some embodiments, HET is a five membered ring heteroaryl optionally substituted with 1-3 R⁸. In some embodiments, HET is a thiazolyl optionally substituted with 1-3 R⁸. In some embodiments, HET is substituted with alkyl (e.g., a methyl), aminoalkyl (e.g., aminomethyl), alkylthiol (e.g., methylthiol), hydroxyalkyl (e.g., hydroxymethyl), alkoxy (e.g., methoxy) or aryl (e.g., phenyl). In some embodiments, HET is substituted with alkyl (e.g., methyl) or aminoalkyl.

In some embodiments, HET is

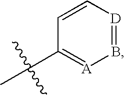

wherein each of A, B and D is independently CH or N. In some embodiments, A is N, B is CH and D is CH. In some embodiments, A is CH, B is N and D is CH. In some embodiments, A is CH, B is CH and D is N.

In some embodiments, HET is

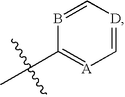

wherein each of A, B and D is independently CH or N. In some embodiments, A is N, B is N and D is CH. In some embodiments, A is N, B is CH and D is N. In some embodiments, A is CH, B is CH and D is CH.

In some embodiments, HET is

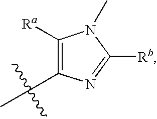

wherein each R^{a} and R^{b} is independently —H or —SMe.

In some embodiments, HET is

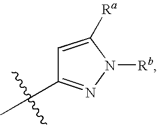

wherein each R^{a} is H, alkyl or —Salkyl; and R^{b} is H, alkyl (e.g., methyl) or aryl (e.g., phenyl).

In some embodiments, HET is

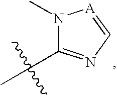

wherein A is CH or N.

In some embodiments, HET is

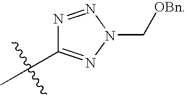

In some embodiments, HET is

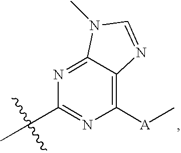

wherein A is S or O.

In some embodiments, HET is

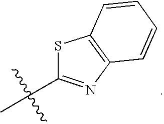

In some embodiments R² is H.

In some embodiments, R² is alkyl (e.g., methyl). In some embodiments, R¹ and R², when taken together with the carbon to which they are attached, form an aryl or a heteroaryl moiety optionally substituted with 1-3 R⁸. In some embodiments, the heteroaryl moiety is a bicyclic heteroaryl moiety.

In some embodiments, R¹ and R², when taken together with the carbon to which they are attached, are

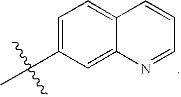

In some embodiments, R¹ and R², when taken together with the carbon to which they are attached, are

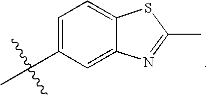

In some embodiments, R¹ and R², when taken together with the carbon to which they are attached, are

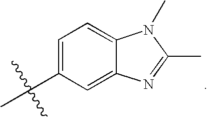

In some embodiments, R¹ and R², when taken together with the carbon to which they are attached, are

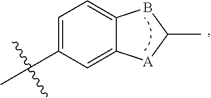

wherein A is N and B is S or wherein A is S and B is N.

In some embodiments, R¹ and R², when taken together with the carbon to which they are attached, are

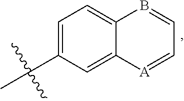

wherein A is N and B is CH or wherein A is CH and B is N.

In some embodiments,

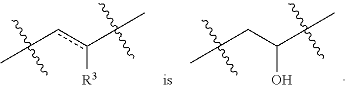

In some embodiments,

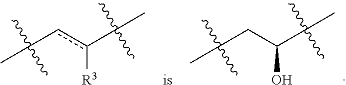

In some embodiments,

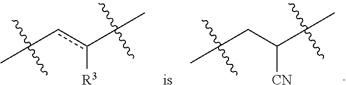

In some embodiments,

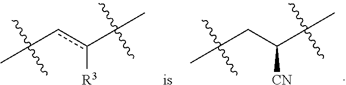

In some embodiments,

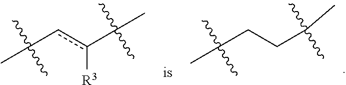

In some embodiments,

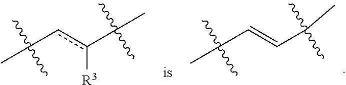

In some embodiments, X is O.

In some embodiments, X is NR⁴ (e.g., NH).

In some embodiments, Y is CR⁵R⁶.

In some embodiments, Y is

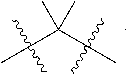

In some embodiments, Y is CH₂.

In some embodiments, Y is NR⁷ (e.g., NH or NMe).

In some embodiments, Q-Z, when taken together, form

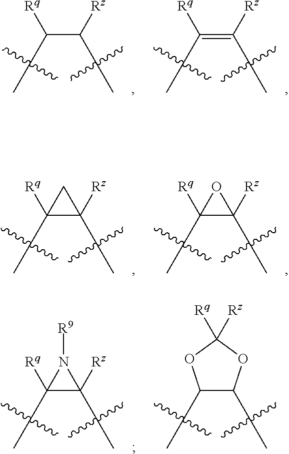

or heteroarylenyl.

In some embodiments, Q-Z, when taken together, form

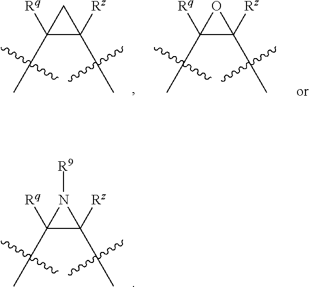

In some embodiments, Q-Z, when taken together, form

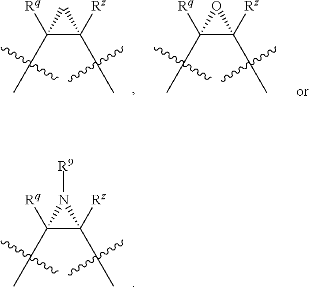

In some embodiments, Q-Z, when taken together, form

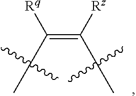

wherein R^{q} is H and R^{z} is H or alkyl (e.g., methyl).

In some embodiments, Q-Z, when taken together, form

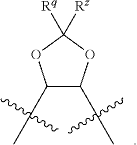

In some embodiments, both R^{q} and R^{z} are methyl.

In some embodiments

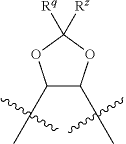

is selected from

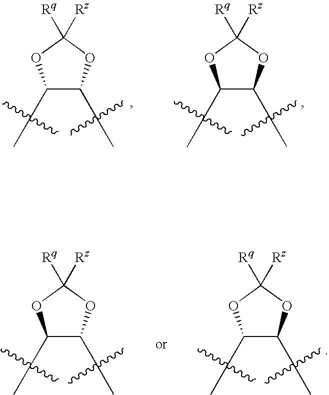

In some embodiments, both R^{q} and R^{z} are methyl.

In some embodiments, Q-Z, when taken together, form a heteroarylenyl. In some embodiments, Q-Z, when taken together, form

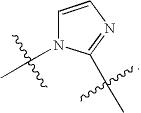

In some embodiments, Q-Z, when taken together, form C(O)NR⁴. In some embodiments, R⁴ is H or alkyl (e.g., methyl or ethyl).

In some embodiments, Q-Z, when taken together, form NR⁴C(O). In some embodiments, R⁴ is H or alkyl (e.g., methyl or ethyl).

In some embodiments, Q-Z, when taken together, form CH₂NR⁴. In some embodiments, R⁴ is H, alkyl, —C(O)Oalkyl, —C(O)Oarylalkyl, —C(O)alkyl, —C(O)aryl or arylalkyl. In some embodiments, R⁴ is —C(O)Oalkyl, —C(O)Oarylalkyl, —C(O)alkyl, —C(O)aryl or arylalkyl.

In some embodiments, Q-Z, when taken together, form NR⁴CH₂. In some embodiments, R⁴ is H, alkyl, —C(O)Oalkyl, —C(O)Oarylalkyl, —C(O)alkyl, —C(O)aryl or arylalkyl. In some embodiments, R⁴ is —C(O)Oalkyl, —C(O)Oarylalkyl, —C(O)alkyl, —C(O)aryl or arylalkyl.

In some embodiments, n is 0.

In some embodiments, n is 1. In some embodiments, the compound of formula (XI) is a compound of formula (XIa)

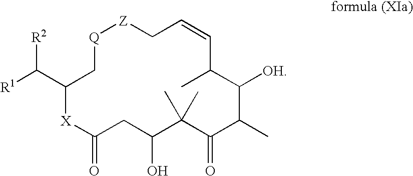

In some embodiments, the compound of formula (XI) is a compound of formula (XIb)

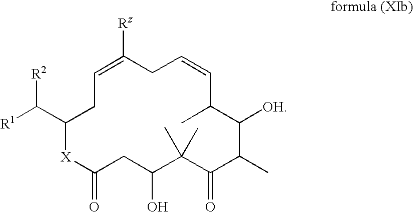

In some embodiments, the compound of formula (XI) is a compound of formula (XIc)

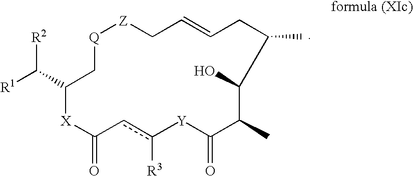

In some embodiments, the compound of formula (XI) is a compound of formula (XId)

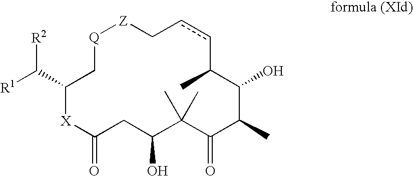

wherein

R¹ is heteroarylalkenyl, which is optionally substituted with 1-3 R⁸;

R² is alkyl (e.g., methyl); or

R¹ and R², when taken together with the carbon to which they are attached, form a heteroaryl moiety substituted with 1 R⁸;

X is O or NR⁴;

R⁴ is H;

Y is CR⁵R⁶;

each of R⁵ and R⁶ is independently alkyl (e.g., methyl);

R⁸ is alkyl (e.g., methyl);

Q-Z, when taken together, form

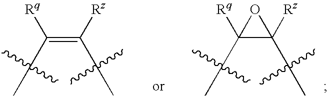

R^{q} is H or alkyl (e.g., methyl);

R^{z} is H or alkyl (e.g., methyl); and

[symbol] is a single or double bond.

In some embodiments, the epothilone is a compound of formula (XII)

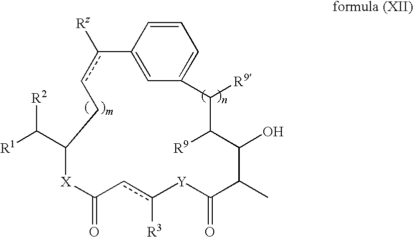

wherein,

R¹ is aryl, heteroaryl, arylalkenyl or heteroarylalkenyl; each of which is optionally substituted with 1-3 R⁸;

R² is H or alkyl (e.g., methyl); or

R¹ and R², when taken together with the carbon to which they are attached, form an aryl or a heteroaryl moiety optionally substituted with 1-3 R⁸;

R³ is H, OH, NH₂, or CN;

X is O or NR⁴;

R⁴ is H, alkyl, —C(O)Oalkyl, —C(O)Oarylalkyl, —C(O)NR⁵alkyl, —C(O) NR⁵arylalkyl, —C(O)alkyl, —C(O)aryl or arylalkyl;

Y is CR⁵R⁶, O or NR⁷;

each of R⁵ and R⁶ is independently H or alkyl (e.g., methyl);

R⁷ is H, alkyl, —C(O)Oalkyl, —C(O)Oarylalkyl, —C(O)NR⁵alkyl, —C(O) NR⁵arylalkyl, —C(O)alkyl, —C(O)aryl or arylalkyl;

each R⁸, for each occurrence, is independently alkyl, aminoalkyl or hydroxyalkyl;

each R⁹ and R^{9′} is independently H or alkyl (e.g., methyl);

R^{z} is H, alkyl (e.g., methyl), haloalkyl (e.g., CF₃), heterocyclylalkyl or N₃;

each [symbol] for each occurrence, is independently a single or double bond;

m is 0, 1 or 2; and

n is 0, 1 or 2.

In some embodiments, R¹ is

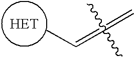

optionally substituted with 1-3 R⁸. In some embodiments, HET is a five membered ring heteroaryl optionally substituted with 1-3 R⁸. In some embodiments, HET is thiazolyl optionally substituted with 1-3 R⁸. In some embodiments, HET is substituted with alkyl (e.g., methyl), aminoalkyl (e.g., aminomethyl), alkylthiol (e.g., methylthiol), hydroxyalkyl (e.g., hydroxymethyl), alkoxy (e.g., methoxy) or aryl (e.g., phenyl). In some embodiments, HET is substituted with alkyl (e.g., methyl) or amino alkyl.

In some embodiments, HET is

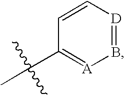

wherein each of A, B and D is independently CH or N. In some embodiments, A is N, B is CH and D is CH. In some embodiments, A is CH, B is N and D is CH. In some embodiments, A is CH, B is CH and D is N.

In some embodiments, HET is

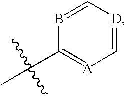

wherein each of A, B and D is independently CH or N. In some embodiments, A is N, B is N and D is CH. In some embodiments, A is N, B is CH and D is N. In some embodiments, A is CH, B is CH and D is CH.

In some embodiments, HET is

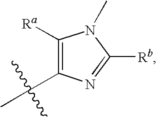

wherein each R^{a} and R^{b} is independently H or —SMe.

In some embodiments, HET is

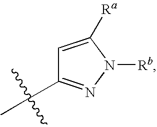

wherein each R^{a} is H, an alkyl or —Salkyl; and R^{b} is H, alkyl (e.g., methyl) or aryl (e.g., phenyl).

In some embodiments, HET is

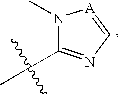

wherein A is CH or N.

In some embodiments, HET is

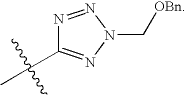

In some embodiments, HET is

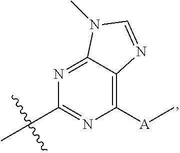

wherein A is S or O.

In some embodiments, HET is

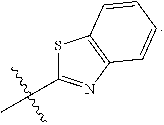

In some embodiments R² is H.

In some embodiments, R² is alkyl (e.g., methyl).

In some embodiments, R¹ and R², when taken together with the carbon to which they are attached, form an aryl or a heteroaryl moiety optionally substituted with 1-3 R⁸.

In some embodiments, the heteroaryl moiety is a bicyclic heteroaryl moiety.

In some embodiments, R¹ and R², when taken together with the carbon to which they are attached, are

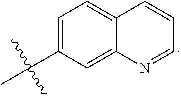

In some embodiments, R¹ and R², when taken together with the carbon to which they are attached, are

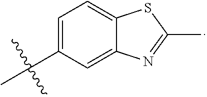

In some embodiments, R¹ and R², when taken together with the carbon to which they are attached, are

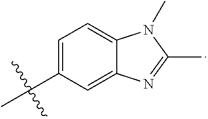

In some embodiments, R¹ and R², when taken together with the carbon to which they are attached, are

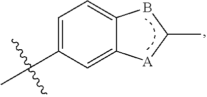

wherein A is N and B is S or wherein A is S and B is N.

In some embodiments, R¹ and R², when taken together with the carbon to which they are attached, are

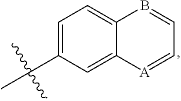

wherein A is N and B is CH or wherein A is CH and B is N.

In some embodiments,

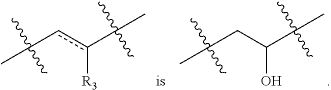

In some embodiments,

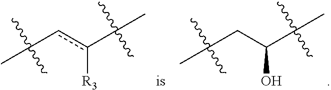

In some embodiments,

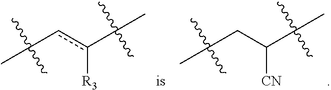

In some embodiments,

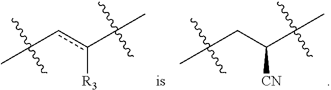

In some embodiments,

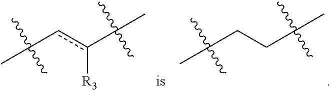

In some embodiments,

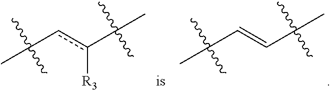

In some embodiments, X is O.

In some embodiments, X is NR⁴ (e.g., NH).

In some embodiments, Y is CR⁵R⁶. In some embodiments, Y is

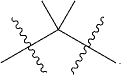

In some embodiments, Y is CH₂.

In some embodiments, Y is NR⁷ (e.g., NH or NMe).

In some embodiments, R⁹ is H.

In some embodiments, R⁹ is Me.

In some embodiments,

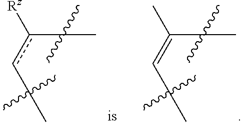

In some embodiments, m is 1.

In some embodiments,

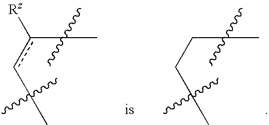

In some embodiments, m is 0.

In some embodiments, n is 0.

In some embodiments,

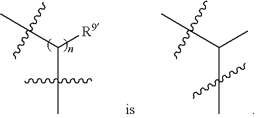

In some embodiments, compound of formula (XII) is a compound of formula (XIIa)

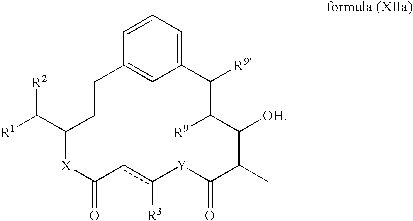

In some embodiments, compound of formula (XII) is a compound of formula (XIIb)

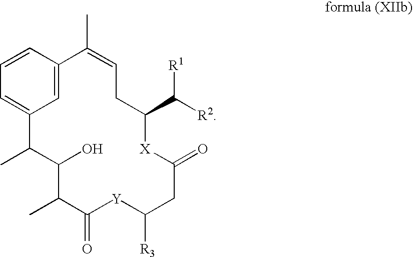

In some embodiments, the epothilone is a compound of formula (XIII):

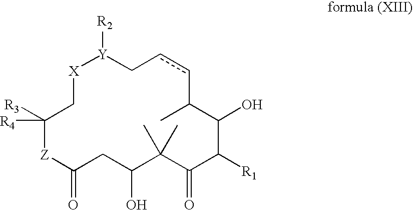

wherein

[symbol] represents a single or double bond;

R₁ is C₁₋₆alkyl, C₂₋₆alkynyl or C₂₋₆alkenyl radical;

R₂ is H or C₁₋₆alkyl radical;

X—Y is selected from the following groups:

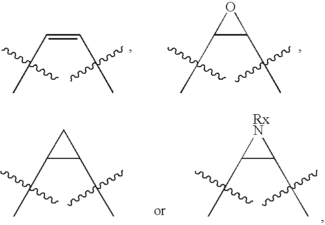

preferably

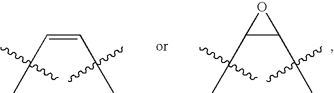

Z is O or NRₓ, wherein Rₓ is hydrogen, alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl group;

R₃ is halogen atom or C₁₋₆alkyl, C₂₋₆alkenyl or C₁₋₆-heteroalkyl radical;

R₄ is bicycloaryl, bicycloheteroaryl or a group of formula —C(R₅)═CHR₆;

R₅ is H or methyl; and

R₆ is an optionally substituted aryl or a heteroaryl group.

In certain embodiments, R₄ is

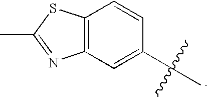

In some embodiments, Z is O. In some embodiments, Z is NH.

In certain embodiments, the compound of formula (XIII) can be represented by the following structures:

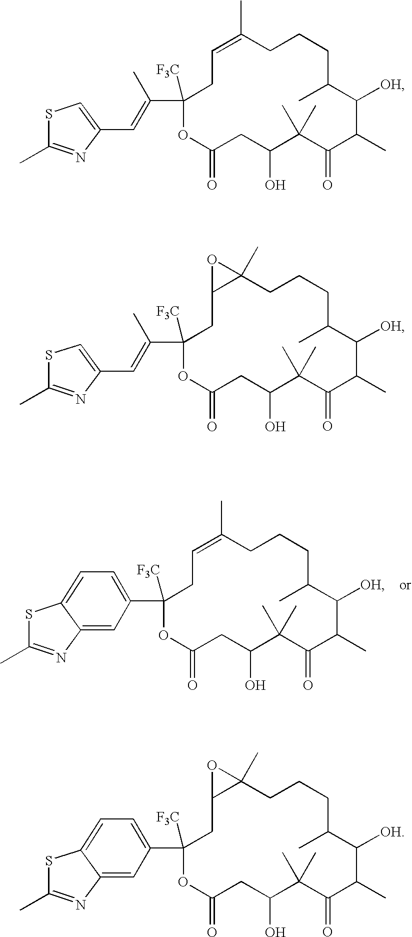

In some embodiments, the epothilone is a compound of formula (XIV):

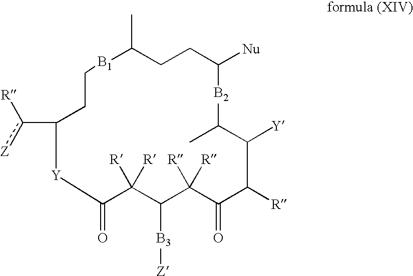

wherein

B₁, B₂, B₃ are selected from single bonds; double bonds in the E(trans) form,

the Z(cis) form or as an E/Z mixture; epoxide rings in the E(trans) form, the Z(cis) form or an E/Z mixture; aziridine rings in the E(trans) form, the Z(cis) form or an E/Z mixture; cyclopropane rings in the E(trans) form, the Z(cis) form or an E/Z mixture; and/or combinations thereof; and being preferably selected from single and double bonds; and particularly preferably being selected from B₁ as Z double bonds or epoxide and B₂ and B₃ as single bond;

R is selected from H, alkyl, aryl, aralkyl (such as —CH₂-aryl, —C₂H₄-aryl and the like), alkenyl (such as vinyl), cycloalkyl (preferably a 3- to 7-membered cycloalkyl), CHₙF₃₋ₙ wherein n=0 to 3, oxacycloalkyl (preferably a 3- to 7-membered oxacycloalkyl) and/or combinations thereof. Preferably R is selected from H, methyl, ethyl, phenyl, benzyl and combinations thereof, and more preferably R is selected from H, methyl, ethyl and combinations thereof;

R′ is selected from the same group as R, and is preferably H;

R″ is selected from the same group as R, and is preferably methyl;

Y is selected from S, NH, N-PG, NR and O; preferably Y is selected from NH, N-PG, NR and O, and more preferably Y is O;

Y′ is selected from H, OH, OR, O-PG, NH₂, NR₂, N(PG)₂, SR and SH; preferably Y′ is O-PG and/or OH;

Nu is selected from R, O-PG, OR, N(PG)₂, NR₂, S-PG, SR, SeR, CN, N₃, aryl and heteroaryl; Nu is preferably selected from R, O-PG, OR, N(PG)₂ and NR₂, and more preferably Nu is H;

Z is selected from —OH, —O-PG, —OR, ═O, ═N—Nu, ═CH-heteroaryl, ═CH-aryl and ═PR₃, where all previously mentioned double bound groups may be present in the E(trans) form, the Z(cis) form or as an E/Z mixture; preferably Z is ═CH-heteroaryl; and more preferably Z is selected from ═O, (E)-(2-methylthiazol-4-yl)-CH═ and (E)-(2-methyloxazol-4-yl)-CH═;

Z′ is selected from 0, OH, OR, O-PG, N(H)₁₋₂, N(R)₁₋₂, N(PG)₁₋₂, SR, S-PG and R; preferably Z′ is O, O-PG and/or OR;

B₃ is selected from single or double bonds in the E(trans) form, the Z(cis) form or as an E/Z mixture; preferably B₃ is selected from single and double bonds with heteroatoms such as O, S and N; and more preferably B₃ is a single bond to O-PG and/or OH;

PG, as referred to herein, is a protecting group, and is preferably selected from allyl, methyl, t-butyl (preferably with electron withdrawing group), benzyl, silyl, acyl and activated methylene derivative (e.g., methoxymethyl), alkoxyalkyl or 2-oxacycloalkyl. Exemplary protecting groups for alcohol and amines include trimethylsilyl, triethylsilyl, dimethyl-tert-butylsilyl, acetyl, propionyl, benzoyl, or a tetrahydropyranyl protecting group. Protecting groups can also be used to protect two neighboring groups (e.g., —CH(OH)—CH(OH)—), or bivalent groups (PG₂). Such protecting groups can form a ring such as a 5- to 7-membered ring. Exemplary protecting groups include succinyl, phthalyl, methylene, ethylene, propylene, 2,2-dimethylpropa-1,3-diyl, and acetonide. Any combination of protecting groups described herein can be used as determined by one of skill in the art.

In some embodiments, the epothilone is a compound of formula (XV):

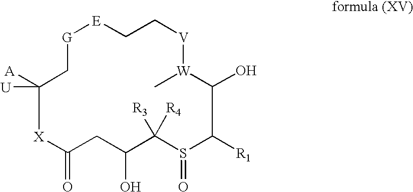

wherein

A is heteroalkyl, heterocycloalkyl, heteroalkylcycloalkyl, heteroaryl, heteroaralkenyl or heteroaralkyl group;

U is hydrogen, halogen, alkyl, heteroalkyl, heterocycloalkyl, heteroalkylcycloalkyl, heteroaryl or heteroaralkyl;

G-E is selected from the following groups,

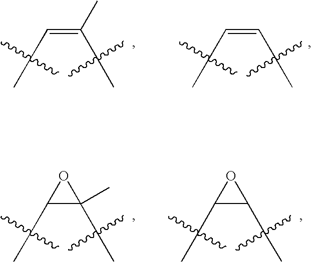

or is part of an optionally substituted phenyl ring;

R₁ is C₁-C₄-alkyl, C₂-C₄-alkenyl, C₂-C₄-alkynyl, or C₃-C₄-cycloalkyl group;

V—W is selected from the group consisting of CH₂CH or CH═C;

X is oxygen or a group of the formula NR₂, wherein R₂ is hydrogen, alkyl, alkenyl, alkynyl, heteroalkyl, aryl, heteroaryl, cycloalkyl, alkylcycloalkyl, heteroalkylcycloalkyl, heterocycloalkyl, aralkyl, or heteroaralkyl; and

each of R₃ and R₄, independently from each other, is hydrogen, C₁-C₄-alkyl or R₃ and R₄ together are part of a cycloalkyl group with 3 or 4 ring atoms.

In certain embodiments of formula (XV), A is a group of Formula (XVII) or (XVIII),

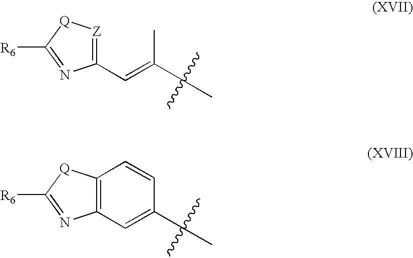

wherein

Q is sulfur, oxygen or NR_{S} (preferably oxygen or sulfur), wherein R₇ is hydrogen, C₁-C₄ alkyl or C₁-C₄ heteroalkyl;

Z is nitrogen or CH (preferably CH); and

R₆ is OR₈, NHR₈, C₁-C₄ alkyl, C₁-C₄ alkenyl, C₁-C₄ alkynyl or C₁-C₆ heteroalkyl (preferably methyl, CH₂OR₈ or CH₂NHR₈), wherein R₈ is hydrogen, C₁-C₄ alkyl or C₁-C₄ heteroalkyl (preferably hydrogen).

In some embodiments, the epothilone is a compound of formula (XVI):

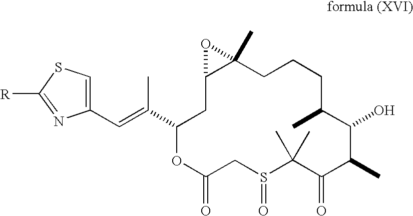

wherein R is selected from OR¹, NHR¹, alkyl, alkenyl, alkynyl and heteroalkyl (e.g., CH₂OR¹ or CH₂NHR¹) and R¹ is selected from hydrogen, C₁₋₄ alkyl and C₁₋₄ heteroalkyl (preferably hydrogen).

In certain embodiments, R is selected from methyl, CH₂OH and CH₂NH₂.

Preparation of naturally occurring and semi-synthetic epothilones and corresponding derivatives is known in the art. Epothilones A & B were first extracted from Sorangium cellulosum So ce90 which exists at the German Collection of Microorganisms as DMS 6773 and DSM 11999. It has been reported that DSM 6773 allegedly displays increased production of epothilones A and B over the wild type strain. Representative fermentation conditions for Sorangium are described, for example, in U.S. Pat. No. 6,194,181 and various international PCT publications including WO 98/10121, WO 97/19086, WO 98/22461 and WO 99/42602. Methods of preparing epothilones are also described in WO 93/10121.

In addition, epothilones can be obtained via de novo synthesis. The total synthesis of epothilones A and B have been reported by a number of research groups including Danishefsky, Schinzer and Nicolaou. These total syntheses are described, for example, in U.S. Pat. Nos. 6,156,905, 6,043,372, and 5,969,145 and in international PCT publications WO 98/08849, WO 98/25929, and WO 99/01124. Additional synthetic methods for making epothilone compounds are also described in PCT publications WO 97/19086, WO 98/38192, WO 99/02514, WO 99/07692, WO 99/27890, WO 99/28324, WO 99/43653, WO 99/54318, WO 99/54319, WO 99/54330, WO 99/58534, WO 59985, WO 99/67252, WO 99/67253, WO 00/00485, WO 00/23452, WO 00/37473, WO 00/47584, WO 00/50423, WO 00/57874, WO 00/58254, WO 00/66589, WO 00/71521, WO 01/07439 and WO 01/27308.

Anti-Cancer Agents

An agent to be delivered using a polymer-agent conjugate, particle or composition described herein may be an anti-cancer agent. Exemplary classes of chemotherapeutic agents include, e.g., the following:

alkylating agents (including, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil Nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexylen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®).

anti-EGFR antibodies (e.g., cetuximab (Erbitux®), panitumumab (Vectibix®), and gefitinib (Iressa®)).

anti-Her-2 antibodies (e.g., trastuzumab (Herceptin®) and other antibodies from Genentech).

antimetabolites (including, without limitation, folic acid antagonists (also referred to herein as antifolates), pyrimidine analogs, purine analogs and adenosine deaminase inhibitors): methotrexate (Rheumatrex®, Trexall®), 5-fluorouracil (Adrucil®, Efudex®, Fluoroplex®), floxuridine (FUDF®), cytarabine (Cytosar-U®, Tarabine PFS), 6-mercaptopurine (Puri-Nethol®)), 6-thioguanine (Thioguanine Tabloid®), fludarabine phosphate (Fludara®), pentostatin (Nipent®), pemetrexed (Alimta®), raltitrexed (Tomudex®), cladribine (Leustatin®), clofarabine (Clofarex®, Clolar®), mercaptopurine (Puri-Nethol®), capecitabine (Xeloda®), nelarabine (Arranon®), azacitidine (Vidaza®) and gemcitabine (Gemzar®). Preferred antimetabolites include, e.g., 5-fluorouracil (Adrucil®, Efudex®, Fluoroplex®), floxuridine (FUDF®), capecitabine (Xeloda®), pemetrexed (Alimta®), raltitrexed (Tomudex®) and gemcitabine (Gemzar®).

vinca alkaloids: vinblastine (Velban®, Velsar®), vincristine (Vincasar®, Oncovin®), vindesine (Eldisine®), vinorelbine (Navelbine®).

platinum-based agents: carboplatin (Paraplat®, Paraplatin®), cisplatin (Platinol®), oxaliplatin (Eloxatin®).

anthracyclines: daunorubicin (Cerubidine®, Rubidomycin®), doxorubicin (Adriamycin®), epirubicin (Ellence®), idarubicin (Idamycin®), mitoxantrone (Novantrone®), valrubicin (Valstar®). Preferred anthracyclines include daunorubicin (Cerubidine®, Rubidomycin®) and doxorubicin (Adriamycin®).

topoisomerase inhibitors: topotecan (Hycamtin®), irinotecan (Camptosar®), etoposide (Toposar®, VePesid®), teniposide (Vumon®), lamellarin D, SN-38, camptothecin (e.g., IT-101).

taxanes: paclitaxel (Taxol®), docetaxel (Taxotere®), larotaxel, cabazitaxel.

antibiotics: actinomycin (Cosmegen®), bleomycin (Blenoxane®), hydroxyurea (Droxia®, Hydrea®), mitomycin (Mitozytrex®, Mutamycin®).

immunomodulators: lenalidomide (Revlimid®), thalidomide (Thalomid®).

immune cell antibodies: alemtuzamab (Campath®), gemtuzumab (Myelotarg®), rituximab (Rituxan®), tositumomab (Bexxar®).

interferons (e.g., IFN-alpha (Alferon®, Roferon-A®, Intron®-A) or IFN-gamma (Actimmune®)).

interleukins: IL-1, IL-2 (Proleukin®), IL-24, IL-6 (Sigosix®), IL-12.

HSP90 inhibitors (e.g., geldanamycin or any of its derivatives). In certain embodiments, the HSP90 inhibitor is selected from geldanamycin, 17-alkylamino-17-desmethoxygeldanamycin (“17-AAG”) or 17-(2-dimethylaminoethyl)amino-17-desmethoxygeldanamycin (“17-DMAG”).

anti-androgens which include, without limitation nilutamide (Nilandron®) and bicalutamide (Caxodex®).

antiestrogens which include, without limitation tamoxifen (Nolvadex®), toremifene (Fareston®), letrozole (Femara®), testolactone (Teslac®), anastrozole (Arimidex®), bicalutamide (Casodex®), exemestane (Aromasin®), flutamide (Eulexin®), fulvestrant (Faslodex®), raloxifene (Evista®, Keoxifene®) and raloxifene hydrochloride.

anti-hypercalcaemia agents which include without limitation gallium (III) nitrate hydrate (Ganite®) and pamidronate disodium (Aredia®).

apoptosis inducers which include without limitation ethanol, 2-[[3-(2,3-dichlorophenoxy)propyl]amino]-(9Cl), gambogic acid, embelin and arsenic trioxide (Trisenox®).

Aurora kinase inhibitors which include without limitation binucleine 2.

Bruton's tyrosine kinase inhibitors which include without limitation terreic acid.

calcineurin inhibitors which include without limitation cypermethrin, deltamethrin, fenvalerate and tyrphostin 8.

CaM kinase II inhibitors which include without limitation 5-Isoquinolinesulfonic acid, 4-[{2S)-2-[(5-isoquinolinylsulfonyl)methylamino]-3-oxo-3-{4-phenyl-1-piperazinyl)propyl]phenyl ester and benzenesulfonamide.

CD45 tyrosine phosphatase inhibitors which include without limitation phosphonic acid.

CDC25 phosphatase inhibitors which include without limitation 1,4-naphthalene dione, 2,3-bis[(2-hydroxyethyl)thio]-(9Cl).

CHK kinase inhibitors which include without limitation debromohymenialdisine.

cyclooxygenase inhibitors which include without limitation 1H-indole-3-acetamide, 1-(4-chlorobenzoyl)-5-methoxy-2-methyl-N-(2-phenylethyl)-(9Cl), 5-alkyl substituted 2-arylaminophenylacetic acid and its derivatives (e.g., celecoxib (Celebrex®), rofecoxib (Vioxx®), etoricoxib (Arcoxia®), lumiracoxib (Prexige®), valdecoxib (Bextra®) or 5-alkyl-2-arylaminophenylacetic acid).

cRAF kinase inhibitors which include without limitation 3-(3,5-dibromo-4-hydroxybenzylidene)-5-iodo-1,3-dihydroindol-2-one and benzamide, 3-(dimethylamino)-N-[3-[(4-hydroxybenzoyl)amino]-4-methylphenyl]-(9Cl).

cyclin dependent kinase inhibitors which include without limitation olomoucine and its derivatives, purvalanol B, roascovitine (Seliciclib®), indirubin, kenpaullone, purvalanol A and indirubin-3′-monooxime.

cysteine protease inhibitors which include without limitation 4-morpholinecarboxamide, N-[(1S)-3-fluoro-2-oxo-1-(2-phenylethyl)propyl]amino]-2-oxo-1-(phenylmethyl)ethyl]-(9Cl).

DNA intercalators which include without limitation plicamycin (Mithracin®) and daptomycin (Cubicin®).

DNA strand breakers which include without limitation bleomycin (Blenoxane®).

E3 ligase inhibitors which include without limitation N-((3,3,3-trifluoro-2-trifluoromethyl)propionyl)sulfanilamide

EGF Pathway Inhibitors which include, without limitation tyrphostin 46, EKB-569, erlotinib (Tarceva®), gefitinib (Iressa®), lapatinib (Tykerb®) and those compounds that are generically and specifically disclosed in WO 97/02266, EP 0 564 409, WO 99/03854, EP 0 520 722, EP 0 566 226, EP 0 787 722, EP 0 837 063, U.S. Pat. No. 5,747,498, WO 98/10767, WO 97/30034, WO 97/49688, WO 97/38983 and WO 96/33980.

farnesyltransferase inhibitors which include without limitation A-hydroxyfarnesylphosphonic acid, butanoic acid, 2-[(2S)-2-[[(2S,3S)-2[[(2R)-2-amino-3-mercaptopropyl]amino]-3-methylpentyl]oxy]-1-oxo-3-phenylpropyl]amino]-4-(methylsulfonyl)-1-methylethylester (2S)-(9Cl), and manumycin A.

Flk-1 kinase inhibitors which include without limitation 2-propenamide, 2-cyano-3-[4-hydroxy-3,5-bis(1-methylethyl)phenyl]-N-(3-phenylpropyl)-(2E)-(9Cl).

glycogen synthase kinase-3 (GSK3) inhibitors which include without limitation indirubin-3′-monooxime.

histone deacetylase (HDAC) inhibitors which include without limitation suberoylanilide hydroxamic acid (SAHA), [4-(2-amino-phenylcarbamoyl)-benzyl]-carbamic acid pyridine-3-ylmethylester and its derivatives, butyric acid, pyroxamide, trichostatin A, oxamflatin, apicidin, depsipeptide, depudecin, trapoxin and compounds disclosed in WO 02/22577.

I-kappa B-alpha kinase inhibitors (IKK) which include without limitation 2-propenenitrile, 3-[(4-methylphenyl)sulfonyl]-(2E)-(9Cl).

imidazotetrazinones which include without limitation temozolomide

(Methazolastone®, Temodar® and its derivatives (e.g., as disclosed generically and specifically in U.S. Pat. No. 5,260,291) and Mitozolomide.

insulin tyrosine kinase inhibitors which include without limitation hydroxyl-2-naphthalenylmethylphosphonic acid.

c-Jun-N-terminal kinase (JNK) inhibitors which include without limitation pyrazoleanthrone and epigallocatechin gallate.

mitogen-activated protein kinase (MAP) inhibitors which include without limitation benzenesulfonamide, N-[2-[[[3-(4-chlorophenyl)-2-propenyl]methyl]amino]methyl]phenyl]-N-(2-hydroxyethyl)-4-methoxy-(9Cl).

MDM2 inhibitors which include without limitation trans-4-iodo, 4′-boranyl-chalcone.

MEK inhibitors which include without limitation butanedinitrile, bis[amino[2-aminophenyl)thio]methylene]-(9Cl).

MMP inhibitors which include without limitation Actinonin, epigallocatechin gallate, collagen peptidomimetic and non-peptidomimetic inhibitors, tetracycline derivatives marimastat (Marimastat®), prinomastat, incyclinide (Metastat®), shark cartilage extract AE-941 (Neovastat®), Tanomastat, TAA211, MMI270B or AAJ996.

mTor inhibitors which include without limitation rapamycin (Rapamune®), and analogs and derivatives thereof, AP23573 (also known as ridaforolimus, deforolimus, or MK-8669), CCI-779 (also known as temsirolimus) (Torisel®) and SDZ-RAD.

NGFR tyrosine kinase inhibitors which include without limitation tyrphostin AG 879.

p38 MAP kinase inhibitors which include without limitation Phenol, 4-[4-(4-fluorophenyl)-5-(4-pyridinyl)-1H-imidazol-2-yl]-(9Cl), and benzamide, 3-(dimethylamino)-N-[3-[(4-hydroxylbenzoyl)amino]-4-methylphenyl]-(9Cl).

p56 tyrosine kinase inhibitors which include without limitation damnacanthal and tyrphostin 46.

PDGF pathway inhibitors which include without limitation tyrphostin AG 1296, tyrphostin 9, 1,3-butadiene-1,1,3-tricarbonitrile, 2-amino-4-(1H-indol-5-yl)-(9Cl), imatinib (Gleevec®) and gefitinib (Iressa®) and those compounds generically and specifically disclosed in European Patent No.: 0 564 409 and PCT Publication No.: WO 99/03854.

phosphatidylinositol 3-kinase inhibitors which include without limitation wortmannin, and quercetin dihydrate.

phosphatase inhibitors which include without limitation cantharidic acid, cantharidin, and L-leucinamide.

protein phosphatase inhibitors which include without limitation cantharidic acid, cantharidin, L-P-bromotetramisole oxalate, 2(5H)-furanone, 4-hydroxy-5-(hydroxymethyl)-3-(1-oxohexadecyl)-(5R)-(9Cl) and benzylphosphonic acid.

PKC inhibitors which include without limitation 1-H-pyrollo-2,5-dione,3-[1-[3-(dimethylamino)propyl]-1H-indol-3-yl]-4-(1H-indol-3-yl)-(9Cl), Bisindolylmaleimide IX, Sphinogosine, staurosporine, and Hypericin.

PKC delta kinase inhibitors which include without limitation rottlerin.

polyamine synthesis inhibitors which include without limitation DMFO.

PTP1B inhibitors which include without limitation L-leucinamide.

protein tyrosine kinase inhibitors which include, without limitation tyrphostin Ag 216, tyrphostin Ag 1288, tyrphostin Ag 1295, geldanamycin, genistein and 7H-pyrrolo[2,3-d]pyrimidine derivatives as generically and specifically described in PCT Publication No.: WO 03/013541 and U.S. Publication No.: 2008/0139587.

SRC family tyrosine kinase inhibitors which include without limitation PP1 and PP2.

Syk tyrosine kinase inhibitors which include without limitation piceatannol.

Janus (JAK-2 and/or JAK-3) tyrosine kinase inhibitors which include without limitation tyrphostin AG 490 and 2-naphthyl vinyl ketone.

retinoids which include without limitation isotretinoin (Accutane®, Amnesteem®, Cistane®, Claravis®, Sotret®) and tretinoin (Aberel®, Aknoten®, Avita®, Renova®, Retin-A®, Retin-A MICRO®, Vesanoid®).

RNA polymerase II elongation inhibitors which include without limitation 5,6-dichloro-1-beta-D-ribofuranosylbenzimidazole.

serine/Threonine kinase inhibitors which include without limitation 2-aminopurine.

sterol biosynthesis inhibitors which include without limitation squalene epoxidase and CYP2D6.

VEGF pathway inhibitors, which include without limitation anti-VEGF antibodies, e.g., bevacizumab, and small molecules, e.g., sunitinib (Sutent®), sorafinib (Nexavar®), ZD6474 (also known as vandetanib) (Zactima™), SU6668, CP-547632 and AZD2171 (also known as cediranib) (Recentin™).

Examples of chemotherapeutic agents are also described in the scientific and patent literature, see, e.g., Bulinski (1997) J. Cell Sci. 110:3055-3064; Panda (1997) Proc. Natl. Acad. Sci. USA 94:10560-10564; Muhlradt (1997) Cancer Res. 57:3344-3346; Nicolaou (1997) Nature 387:268-272; Vasquez (1997) Mol. Biol. Cell. 8:973-985; Panda (1996) J. Biol. Chem. 271:29807-29812.

In some embodiments, the agent is an anti-cancer agent. An anti-cancer agent may be an alkylating agent (e.g., nitrogen mustards, nitrosoureas, platinum, alkyl sulfonates, hydrazines, triazenes, aziridines, spindle poison, cytotoxic agents, topoisomerase inhibitors and others), a cytotoxic agent, an anti-angiogenic agent, a vascular disrupting agent, a microtubule targeting agent, a mitotic inhibitor, a topoisomerase inhibitor, or an anti-metabolite (e.g., folic acid, purine, and pyrimidine derivatives). Exemplary anti-cancer agents include aclarubicin, actinomycin, alitretinon, altretamine, aminopterin, aminolevulinic acid, amrubicin, amsacrine, anagrelide, arsenic trioxide, asparaginase, atrasentan, belotecan, bexarotene, endamustine, bleomycin, busulfan, camptothecin, capecitabine, carboplatin, carboquone, carmofur, carmustine, celecoxib, chlorambucil, chlormethine, cisplatin, cladribine, clofarabine, crisantaspase, cyclophosphamide, cytarabine, dacarbazine, dactinomycin, daunorubicin, decitabine, demecolcine, docetaxel, doxorubicin, efaproxiral, elesclomol, elsamitrucin, enocitabine, epirubicin, estramustine, etoglucid, etoposide, floxuridine, fludarabine, fluorouracil (5FU), fotemustine, gemcitabine, Gliadel implants, hydroxycarbamide, hydroxyurea, idarubicin, ifosfamide, irinotecan, irofulven, larotaxel, leucovorin, liposomal doxorubicin, liposomal daunorubicin, lonidamine, lomustine, lucanthone, mannosulfan, masoprocol, melphalan, mercaptopurine, mesna, methotrexate, methyl aminolevulinate, mitobronitol, mitoguazone, mitotane, mitomycin, mitoxantrone, nedaplatin, nimustine, oblimersen, omacetaxine, ortataxel, oxaliplatin, paclitaxel, pegaspargase, pemetrexed, pentostatin, pirarubicin, pixantrone, plicamycin, porfimer sodium, prednimustine, procarbazine, raltitrexed, ranimustine, rubitecan, sapacitabine, semustine, sitimagene ceradenovec, strataplatin, streptozocin, talaporfin, tamoxifen, tegafur-uracil, temoporfin, temozolomide, teniposide, tesetaxel, testolactone, tetranitrate, thiotepa, tiazofurine, tioguanine, tipifarnib, topotecan, trabectedin, triaziquone, triethylenemelamine, triplatin, tretinoin, treosulfan, trofosfamide, uramustine, valrubicin, verteporfin, vinblastine, vincristine, vindesine, vinflunine, vinorelbine, vorinostat, zorubicin, and combinations thereof, or other cytostatic or cytotoxic agents described herein.

Agent may mean a combination of agents that have been combined and attached to a polymer and/or loaded into the particle. Any combination of agents may be used. For example, an epothilone may be combined with an anti-cancer agent. In certain embodiments for treating cancer, at least two traditional chemotherapeutic agents are attached to a polymer and/or loaded into the particle.

In certain embodiments, the agent may be attached to a polymer to form a polymer-agent conjugate.

In certain embodiments, the agent in the particle is attached to a polymer of the particle. The agent may be attached to any polymer in the particle, e.g., a hydrophobic polymer or a polymer containing a hydrophilic and a hydrophobic portion.

In certain embodiments, an agent is embedded in the particle. The agent may be associated with a polymer or other component of the particle through one or more non-covalent interactions such as van der Waals interactions, hydrophobic interactions, hydrogen bonding, dipole-dipole interactions, ionic interactions, and pi stacking.

An agent may be present in varying amounts of a polymer-agent conjugate, particle or composition described herein. When present in a particle, the agent may be present in an amount, e.g., from about 1 to about 30% by weight (e.g., from about 2 to about 30% by weight, from about 4 to about 25% by weight, or from about 5 to about 13%, 14%, 15%, 16%, 17%, 18%, 19% or 20% by weight).

Modes of Attachment

An agent described herein may be directly attached to a polymer described herein. A reactive functional group of an agent may be directly attached to a functional group on a polymer. An agent may be attached to a polymer via a variety of linkages, e.g., an amide, ester, succinimide, carbonate or carbamate linkage. For example, in one embodiment, hydroxy group of an agent may be reacted with a carboxylic acid group of a polymer, forming a direct ester linkage between the agent and the polymer. In another embodiment, an amino group of an agent may be linked to a carboxylic acid group of a polymer, forming an amide bond.

In some embodiments, an agent may be directly attached to a terminal end of a polymer. For example, a polymer having a carboxylic acid moiety at its terminus may be covalently attached to a hydroxy or amino moiety of an agent, forming an ester or amide bond.

In certain embodiments, suitable protecting groups may be required on the other polymer terminus or on other reactive substituents on the agent, to facilitate formation of the specific desired conjugate. For example, a polymer having a hydroxy terminus may be protected, e.g., with an alkyl group (e.g., methyl) or an acyl group (e.g., acetyl). An agent such as an epothilone may be protected, e.g., with a protecting group (e.g., tert-butyldimethylsilyl (TBDMS) or 2,2,2-trichlorethoxycarbonyl (Troc)), on the hydroxyl group at the 3 position, such that the epothilone may be attached to a polymer via the hydroxyl group at the 7 position. Alternatively, an epothilone may be protected, e.g., with a protecting group (e.g., tert-butyldimethylsilyl (TBDMS) or 2,2,2-trichlorethoxycarbonyl (Troc)), on the hydroxyl group at the 7 position, such that the epothilone may be attached to a polymer via the hydroxyl group at the 3 position.

In some embodiments, the process of attaching an agent to a polymer may result in a composition comprising a mixture of polymer-agent conjugates having the same polymer and the same agent, but which differ in the nature of the linkage between the agent and the polymer. For example, when an agent has a plurality of reactive moieties that may react with a polymer, the product of a reaction of the agent and the polymer may include a polymer-agent conjugate wherein the agent is attached to the polymer via one reactive moiety, and a polymer-agent conjugate wherein the agent is attached to the polymer via another reactive moiety. For example, epothilones have a plurality of hydroxyl moieties, all of which may react with a polymer. Thus, when the agent is an epothilone, the resulting composition may include a plurality of polymer-epothilone conjugates including polymers attached to the agent via different hydroxyl groups present on the epothilone. For example, the plurality of polymer-agent conjugates may include polymers attached to an epothilone via the hydroxyl group at the 3 position and/or polymers attached to an epothilone via the hydroxyl group at the 7 position.

In some embodiments, the process of attaching an agent to a polymer may involve the use of protecting groups. For example, when an agent has a plurality of reactive moieties that may react with a polymer, the agent may be protected at certain reactive positions such that a polymer will be attached via a specified position. In one embodiment, when the agent is an epothilone, the agent may be selectively coupled to the polymer, e.g., via the hydroxyl group at the 3 position, by protecting the hydroxyl group at the 7 position with a suitable protecting group. Alternatively, the agent may be selectively coupled to the polymer via the hydroxyl group at the 7 position, by protecting the hydroxyl group at the 3 position with a suitable protecting group.

In some embodiments, selectively-coupled products such as those described above may be combined to form mixtures of polymer-agent conjugates. For example, PLGA attached to an epothilone via the hydroxyl group at the 3 position, and PLGA attached to an epothilone via the hydroxyl group at the 7 position, may be combined to form a mixture of the two polymer-agent conjugates, and the mixture may be used in the preparation of a particle.

A polymer-agent conjugate may comprise a single agent attached to a polymer. The agent may be attached to a terminal end of a polymer, or to a point along a polymer chain.

In some embodiments, the polymer-agent conjugate may comprise a plurality of agents attached to a polymer (e.g., 2, 3, 4, 5, 6 or more agents may be attached to a polymer). The agents may be the same or different. In some embodiments, a plurality of agents may be attached to a multifunctional linker (e.g., a polyglutamic acid linker). In some embodiments, a plurality of agents may be attached to points along the polymer chain.

Linkers

An agent may be attached to a polymer via a linker, such as a linker described herein. In certain embodiments, a plurality of the linker moieties are attached to a polymer, allowing attachment of a plurality of agents to the linker. The agent may be released from the linker under biological conditions. In another embodiment a single linker is attached to a polymer, e.g., at a terminus of the polymer.

The linker may be, for example, an alkylenyl (divalent alkyl) group. In some embodiments, one or more carbon atoms of the alkylenyl linker may be replaced with one or more heteroatoms. In some embodiments, one or more carbon atoms may be substituted with a substituent (e.g., alkyl, amino, or oxo substituents).

In some embodiments, the linker, prior to attachment to the agent and the polymer, may have one or more of the following functional groups: amine, amide, hydroxyl, carboxylic acid, ester, halogen, thiol, carbonate, or carbamate.

In some embodiments, the linker may comprise an amino acid linker or a peptide linker. Frequently, in such embodiments, the peptide linker is cleavable by hydrolysis, under reducing conditions, or by a specific enzyme.

When the linker is the residue of a divalent organic molecule, the cleavage of the linker may be either within the linker itself, or it may be at one of the bonds that couples the linker to the remainder of the conjugate, i.e. either to the agent or the polymer.

In some embodiments, a linker may be selected from one of the following:

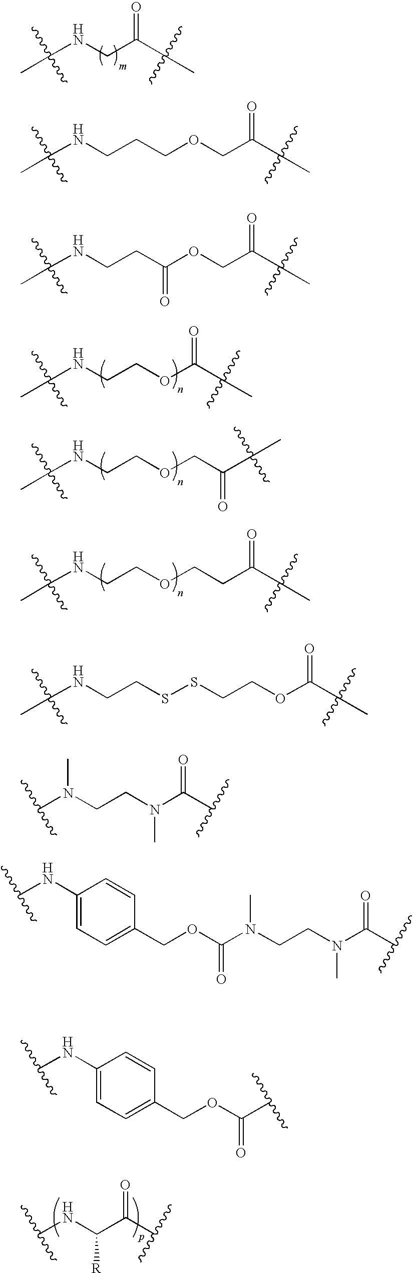

wherein m is 1-10, n is 1-10, p is 1-10, and R is an amino acid side chain.

A linker may be, for example, cleaved by hydrolysis, reduction reactions, oxidative reactions, pH shifts, photolysis, or combinations thereof; or by an enzyme reaction. The linker may also comprise a bond that is cleavable under oxidative or reducing conditions, or may be sensitive to acids.

Methods of Making Polymer-Agent Conjugates

The polymer-agent conjugates may be prepared using a variety of methods known in the art, including those described herein. In some embodiments, to covalently link the agent to a polymer, the polymer or agent may be chemically activated using any technique known in the art. The activated polymer is then mixed with the agent, or the activated agent is mixed with the polymer, under suitable conditions to allow a covalent bond to form between the polymer and the agent. In some embodiments, a nucleophile, such as a thiol, hydroxyl group, or amino group, on the agent attacks an electrophile (e.g., activated carbonyl group) to create a covalent bond. An agent may be attached to a polymer via a variety of linkages, e.g., an amide, ester, succinimide, carbonate or carbamate linkage.

In some embodiments, an agent may be attached to a polymer via a linker. In such embodiments, a linker may be first covalently attached to a polymer, and then attached to an agent. In other embodiments, a linker may be first attached to an agent, and then attached to a polymer.

Exemplary Polymer-Agent Conjugates

Polymer-agent conjugates can be made using many different combinations of components described herein. For example, various combinations of polymers (e.g., PLGA, PLA or PGA), linkers attaching the agent to the polymer, and agents are described herein.

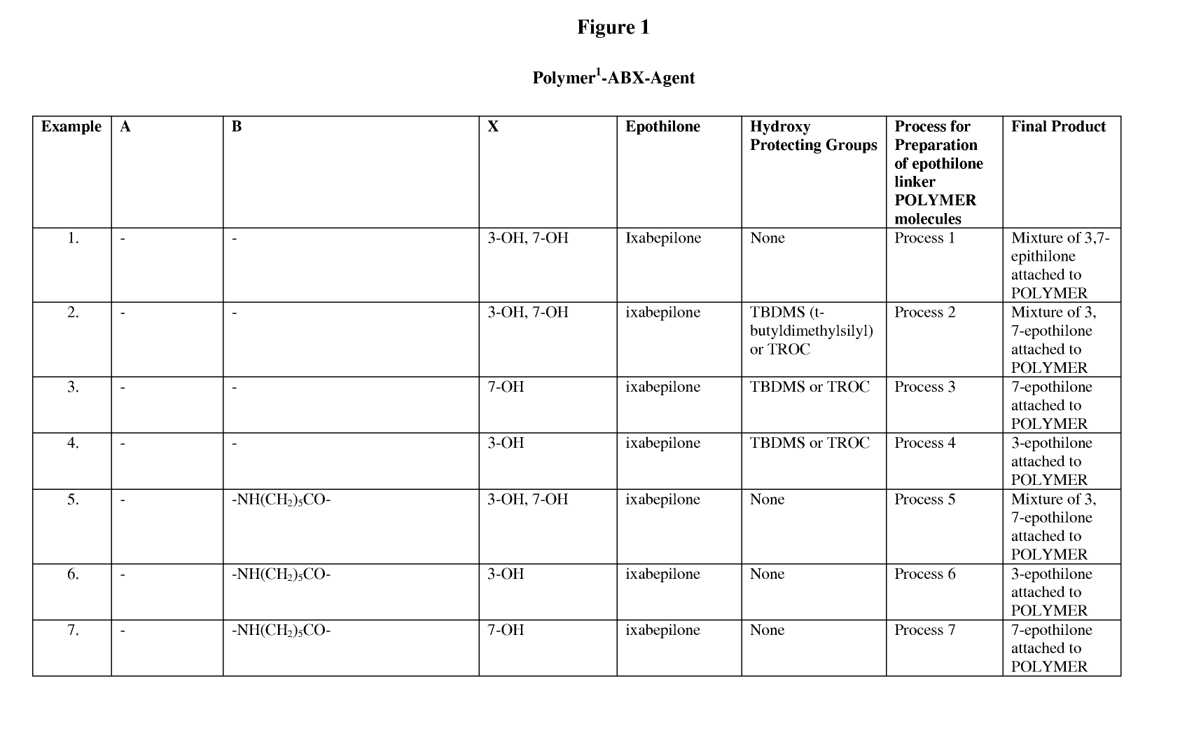

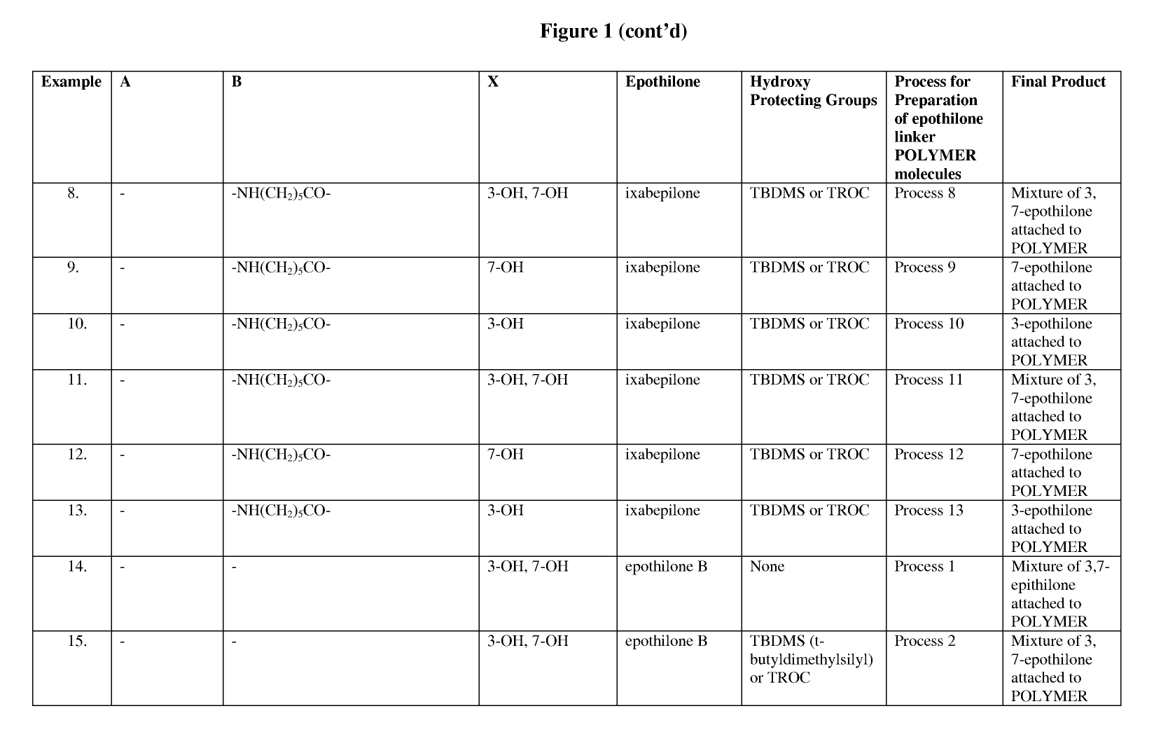

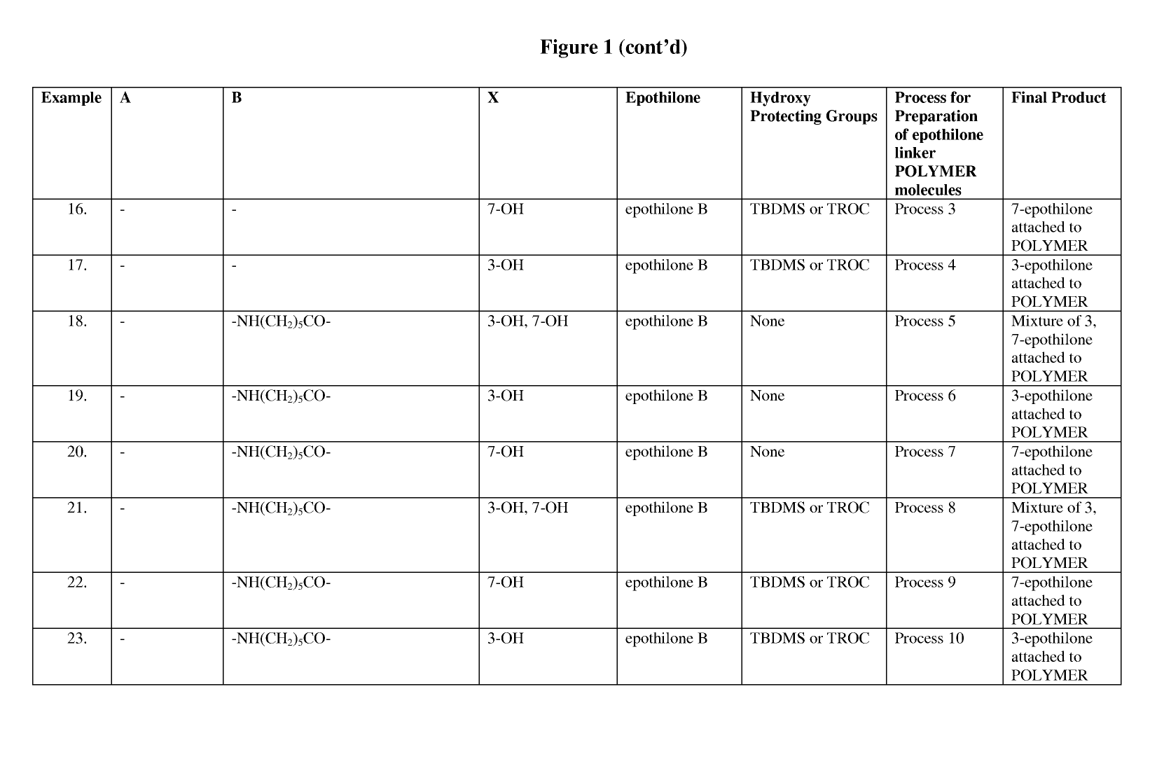

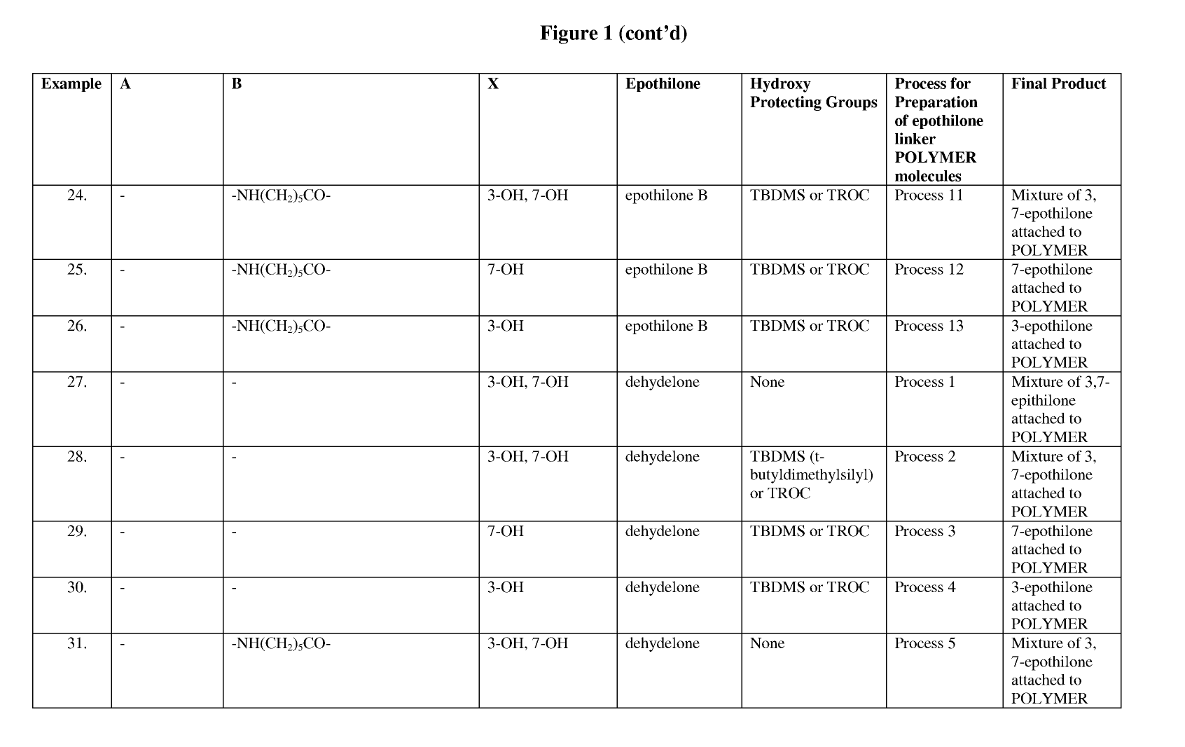

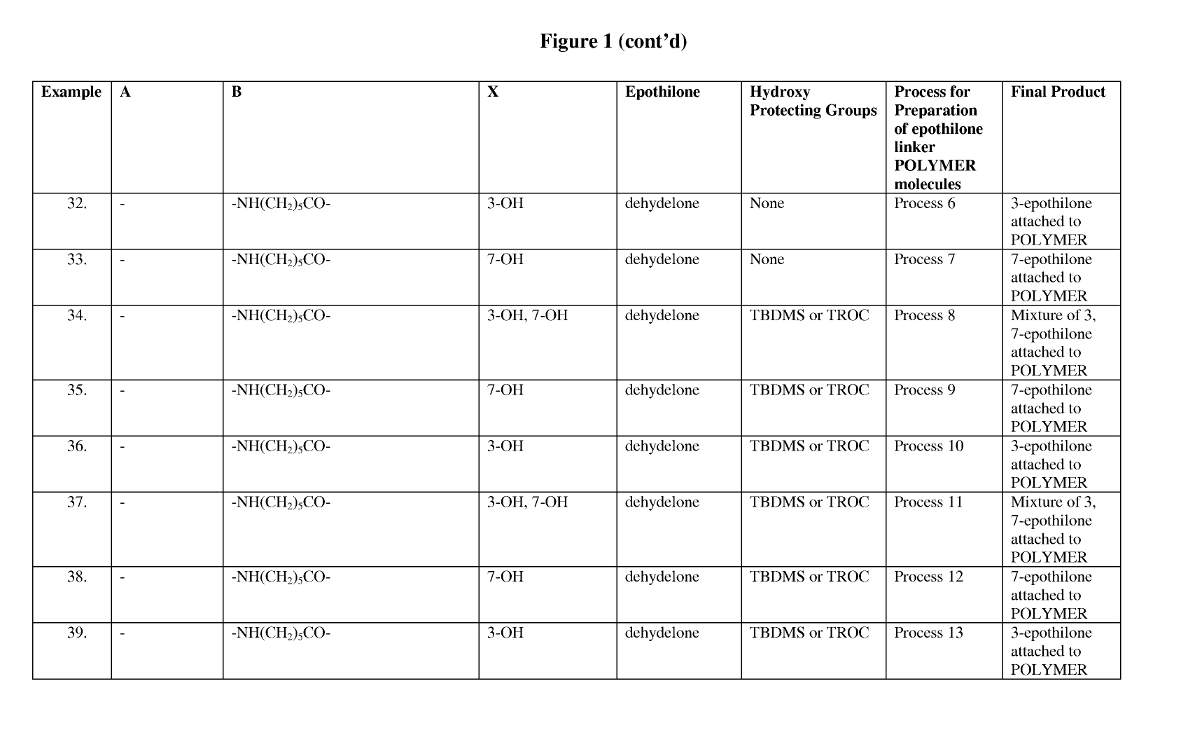

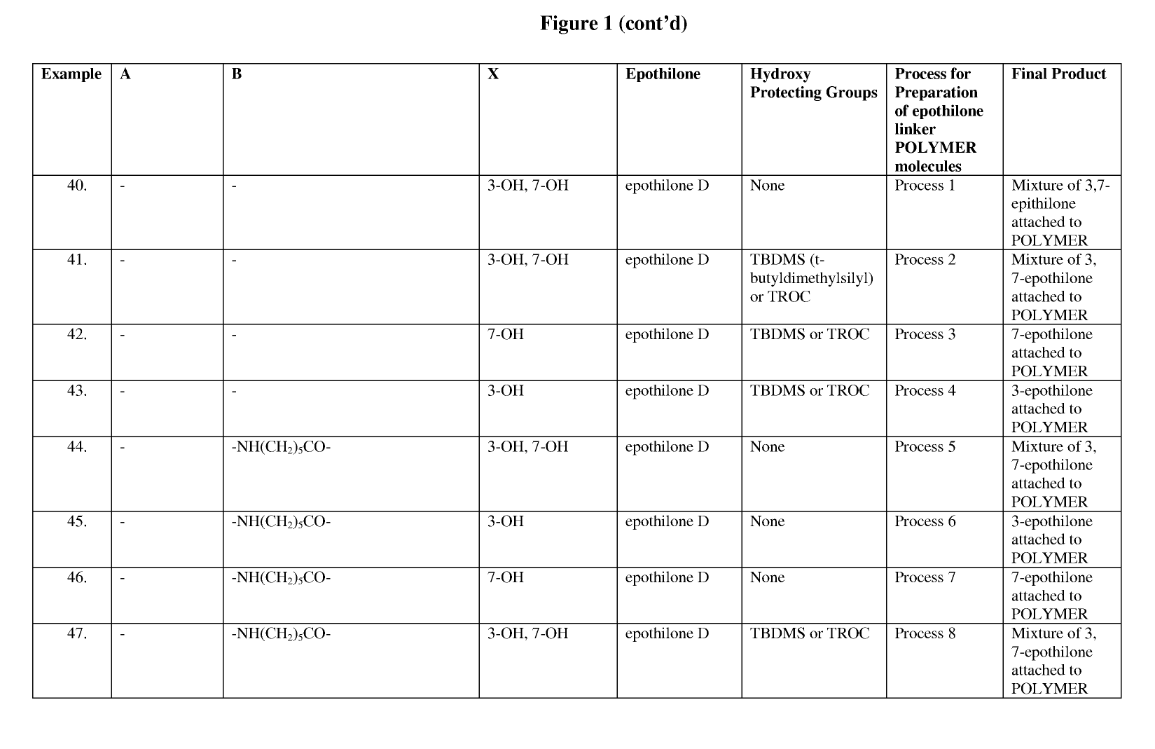

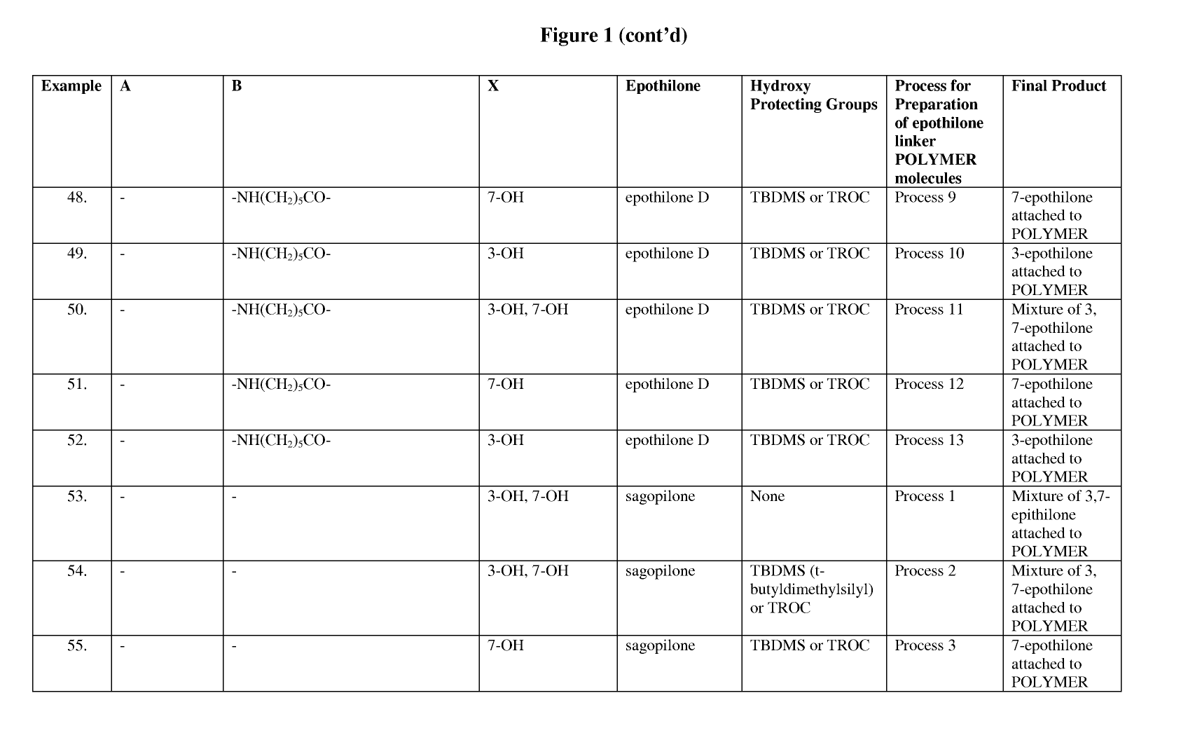

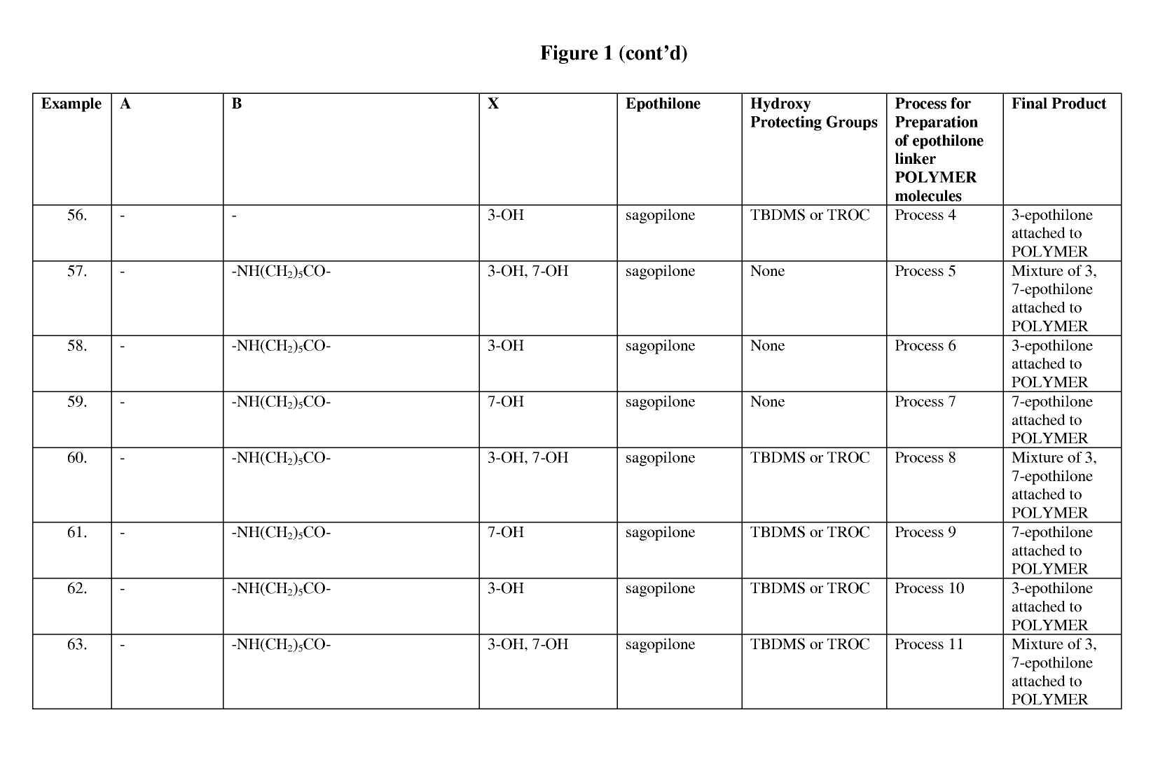

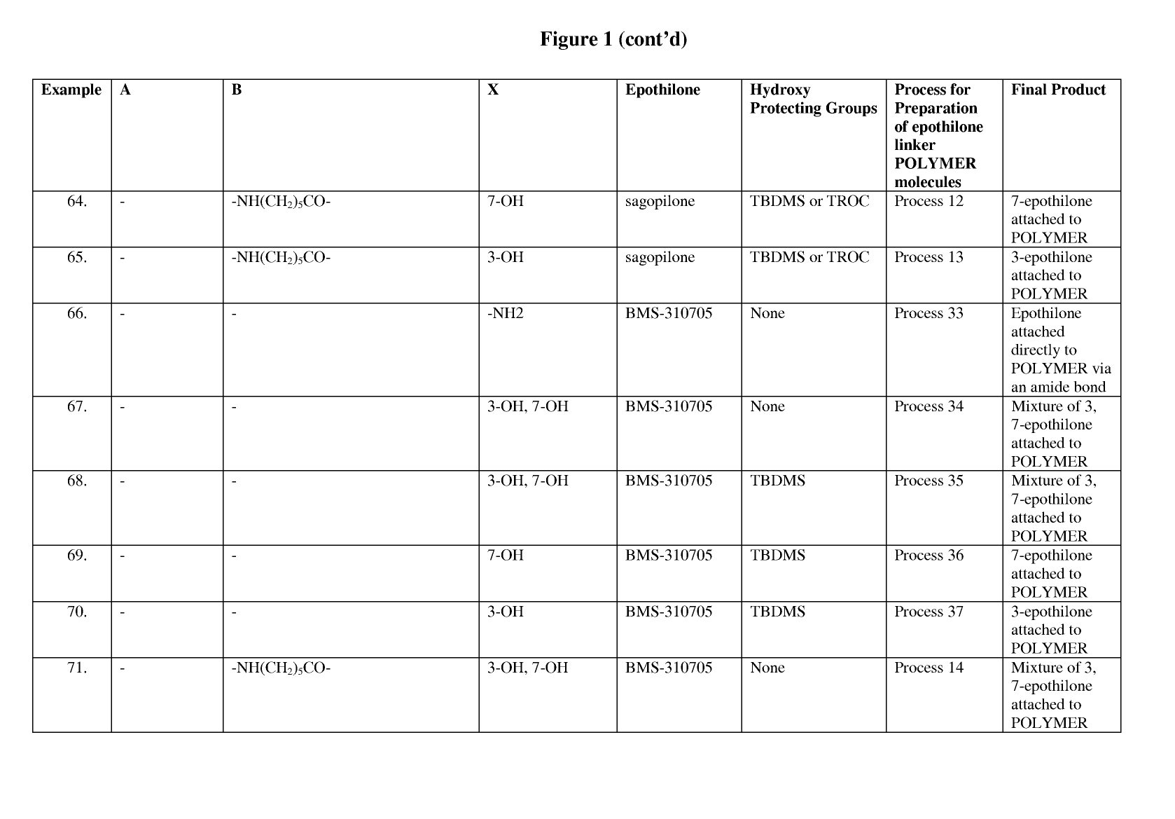

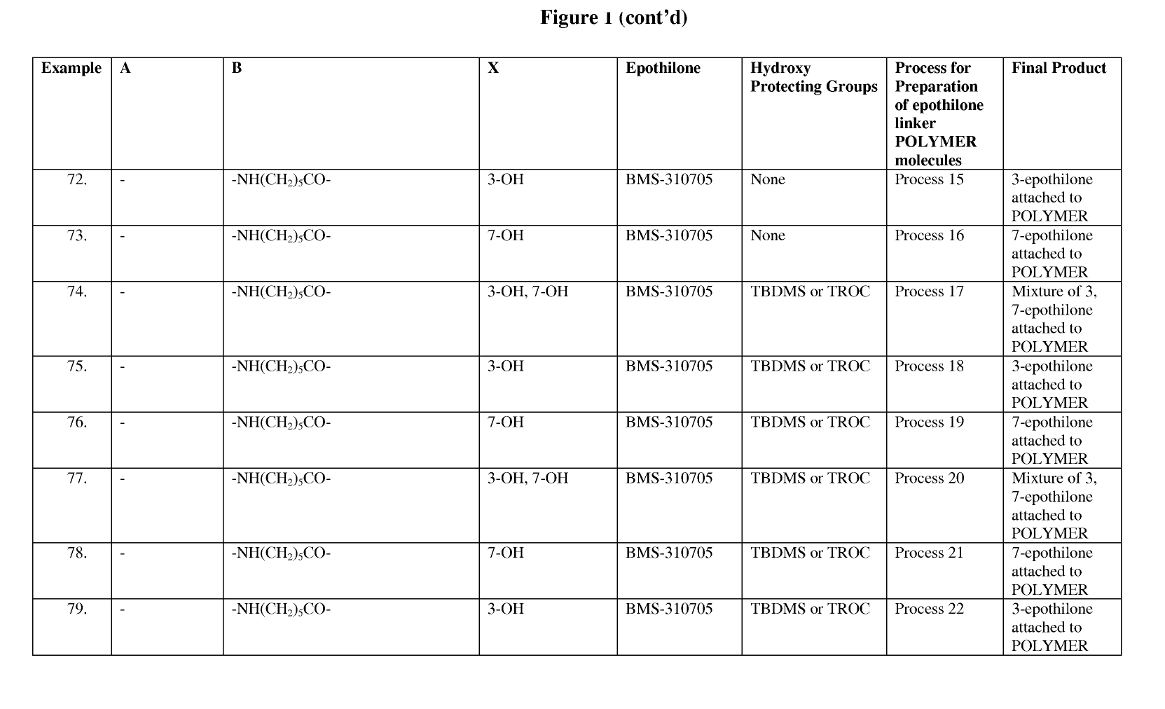

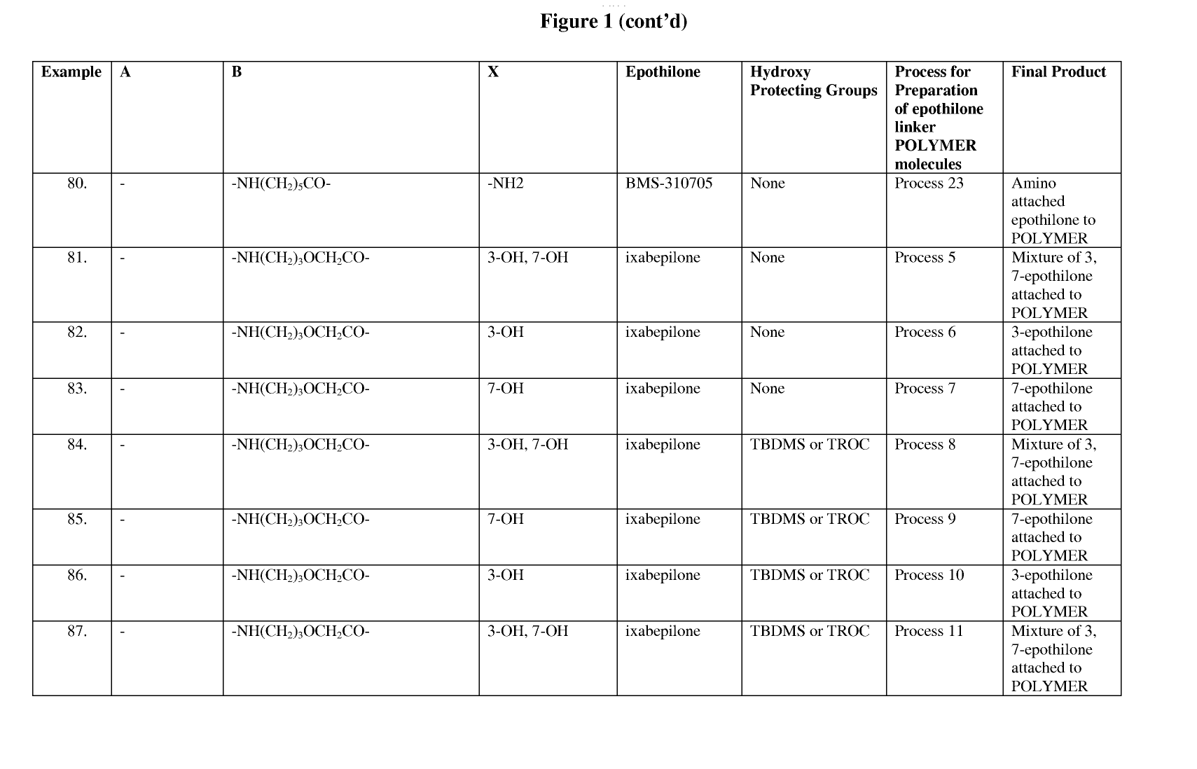

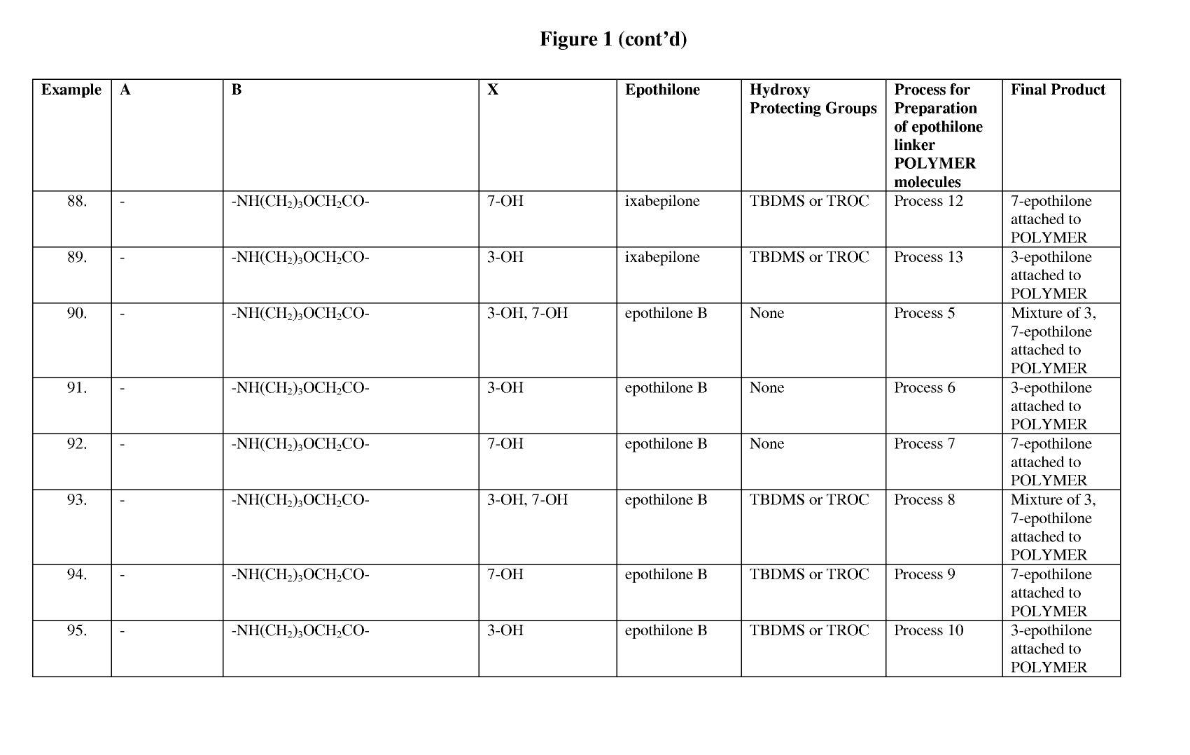

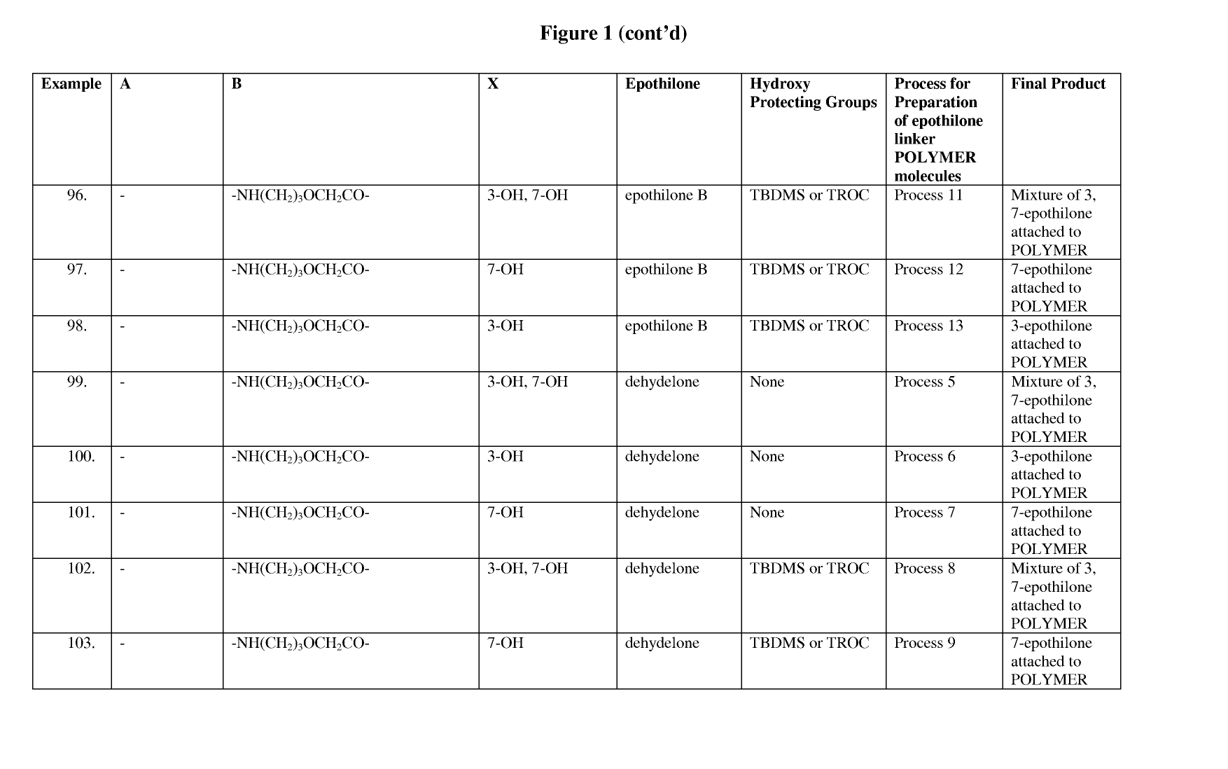

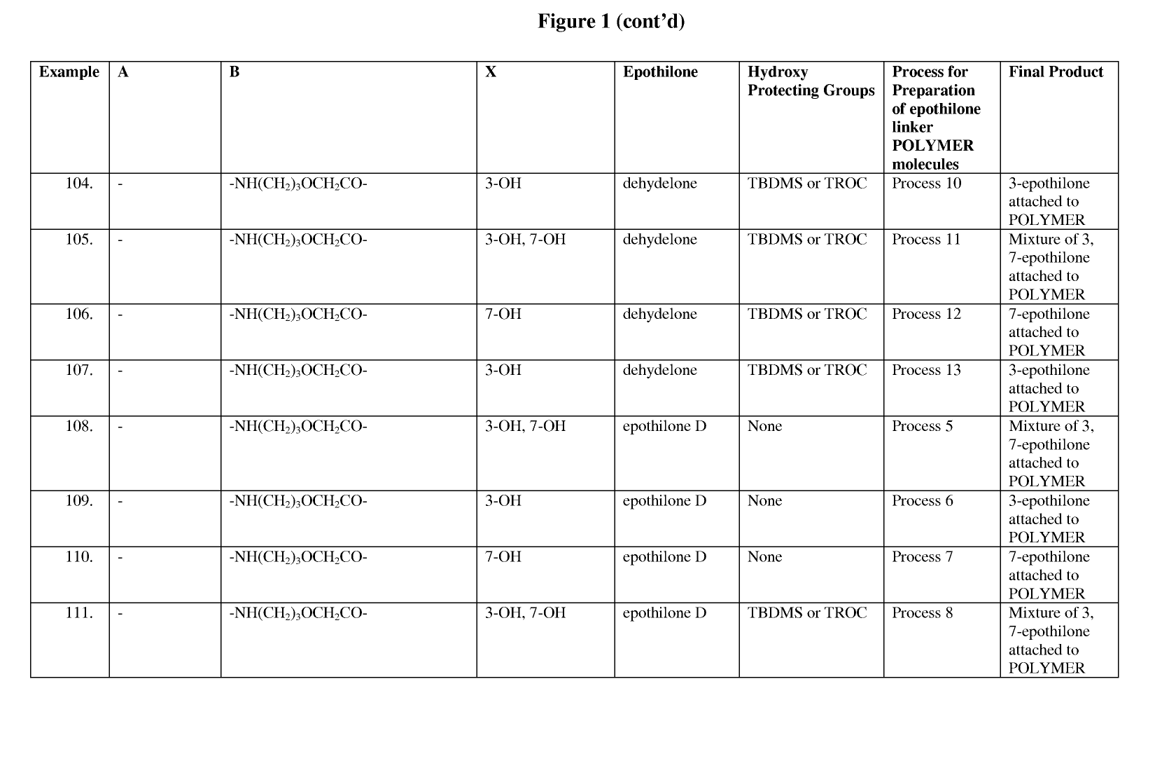

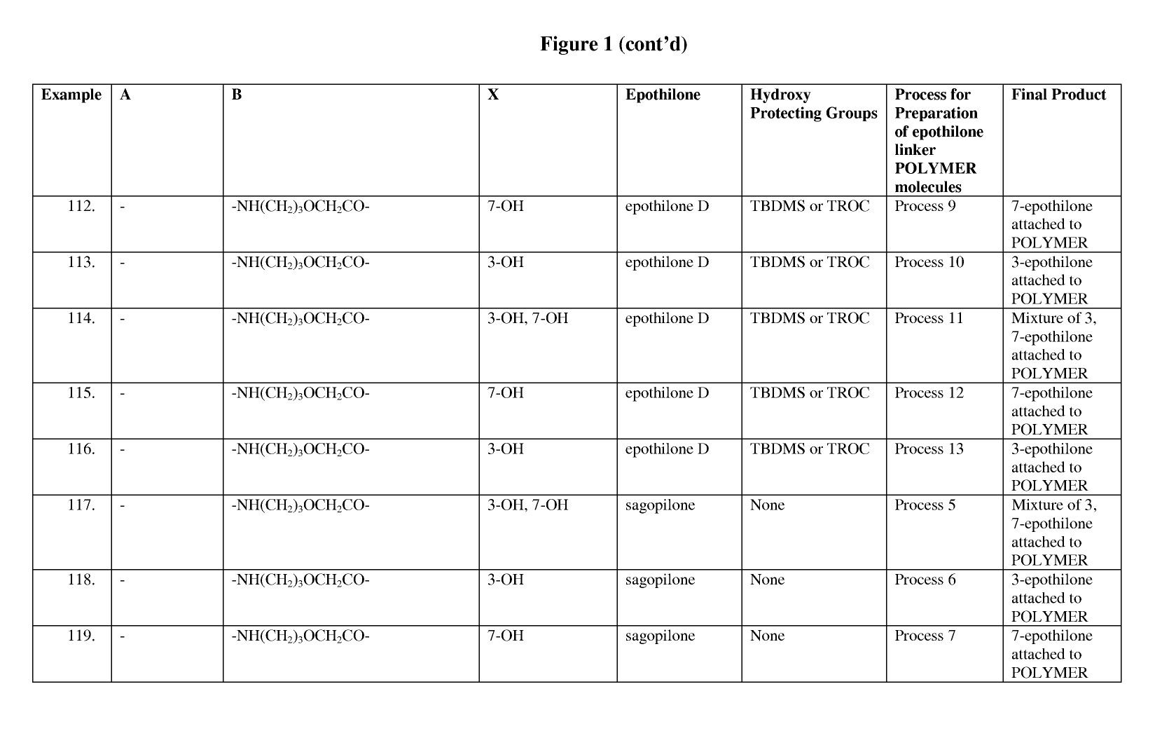

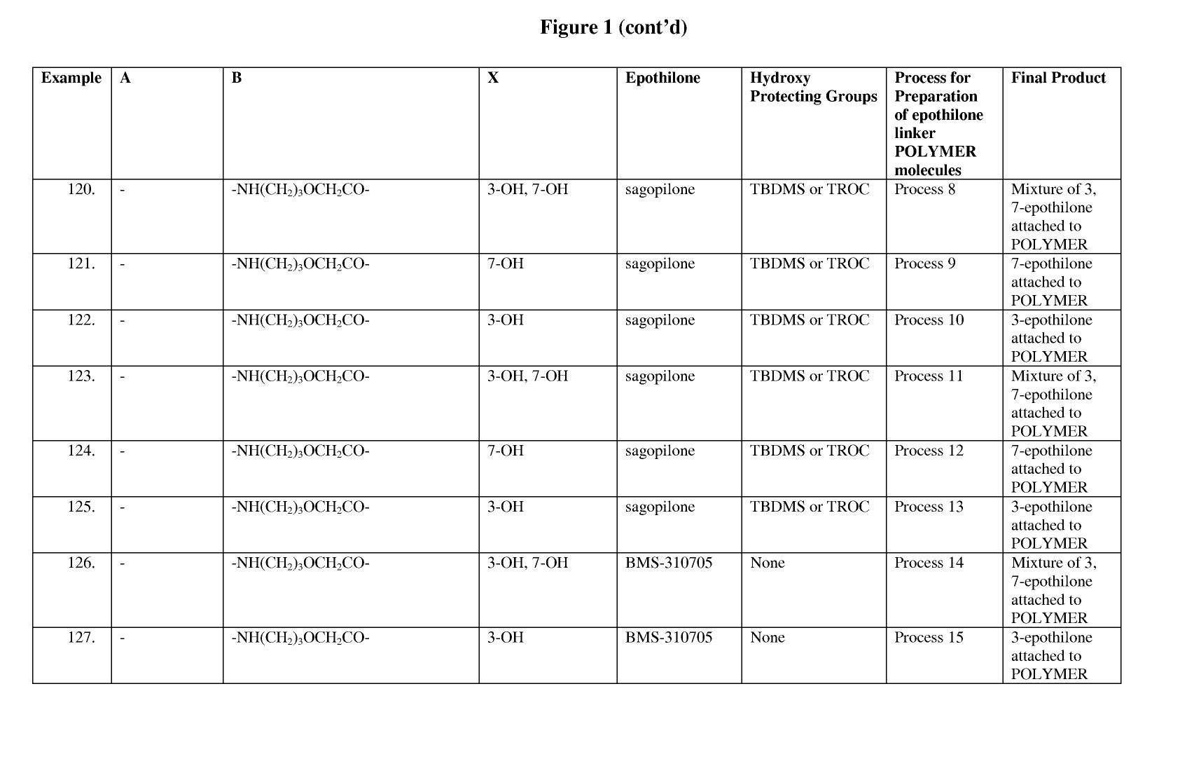

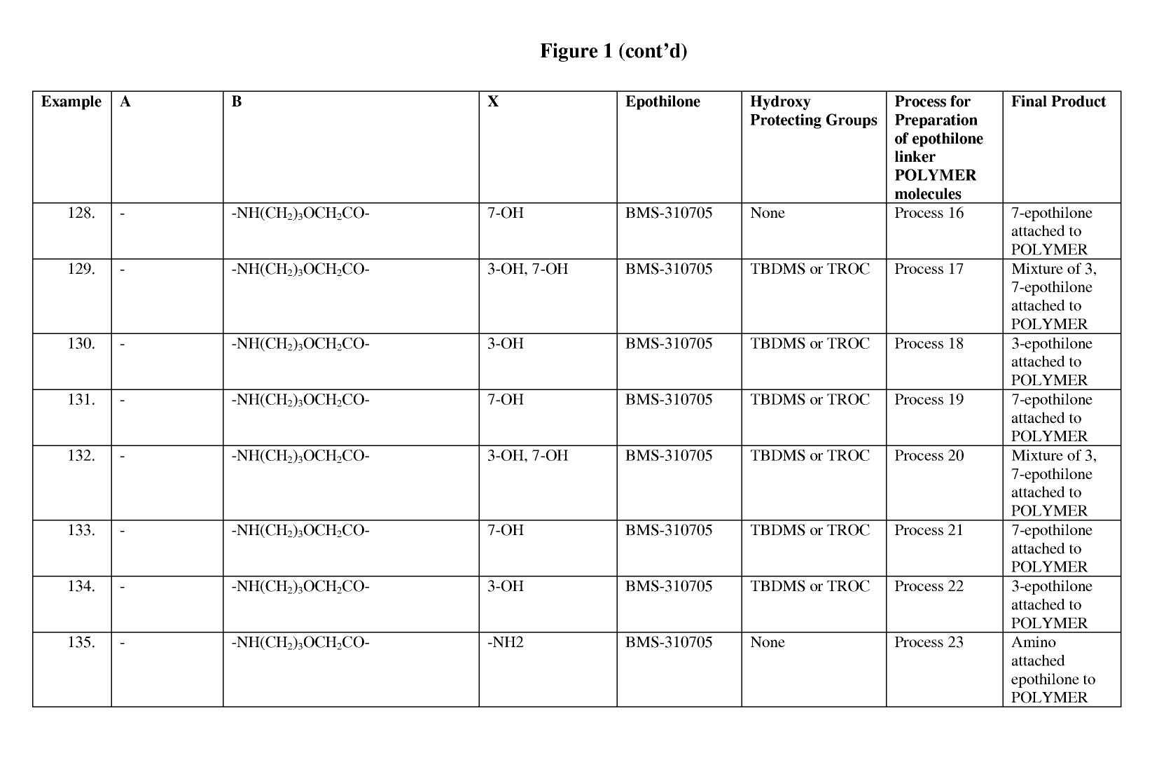

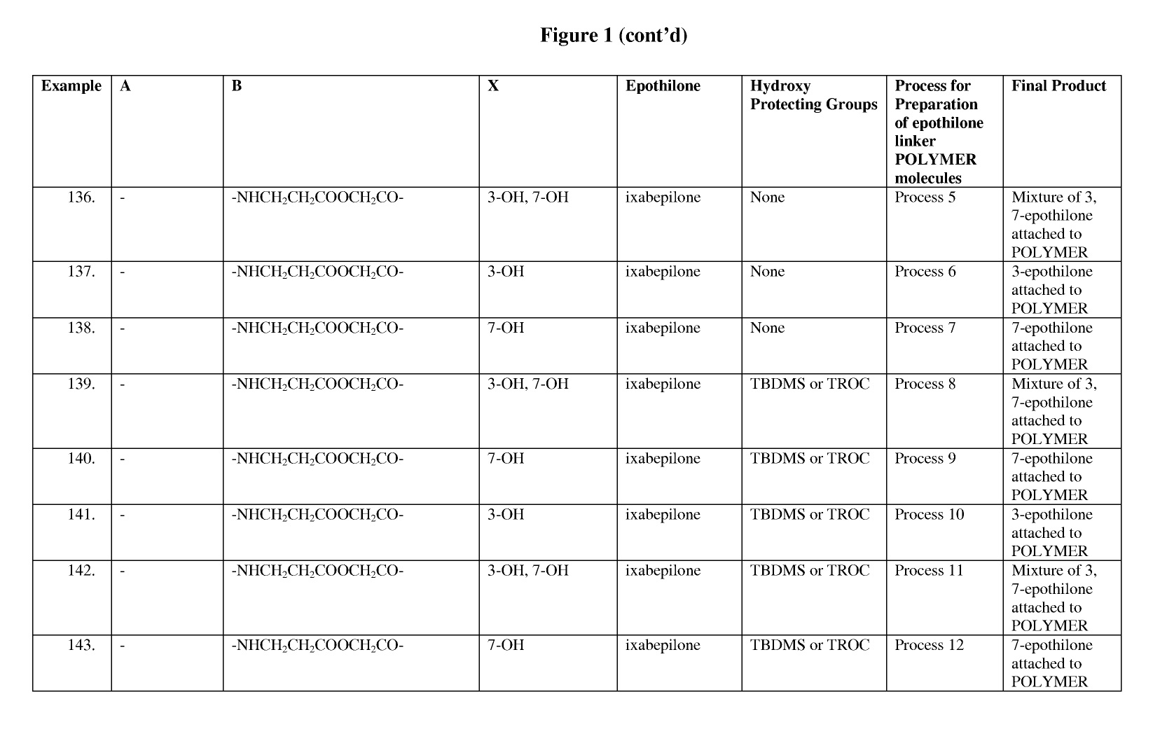

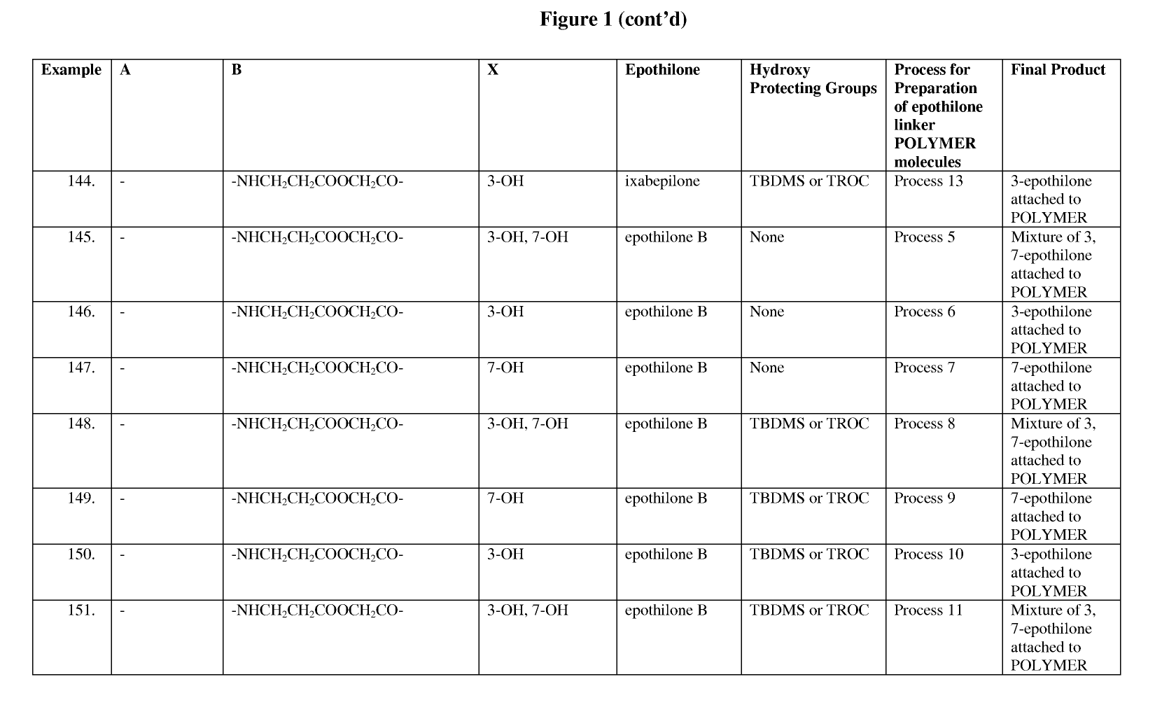

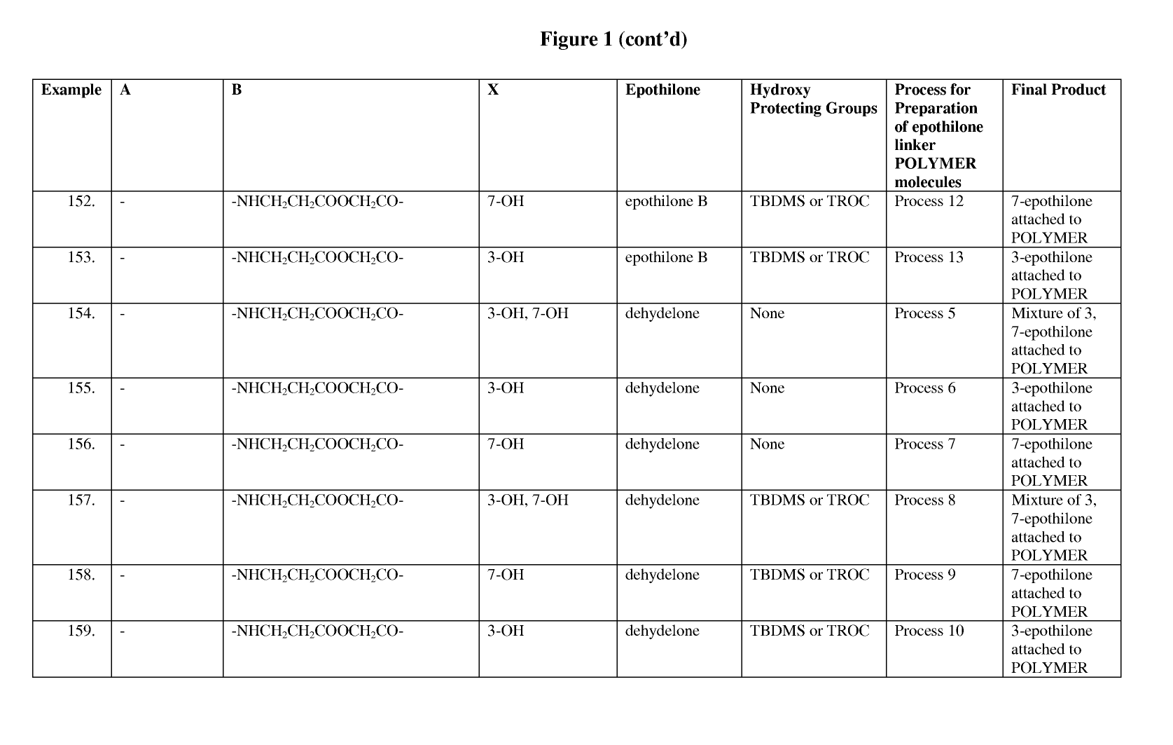

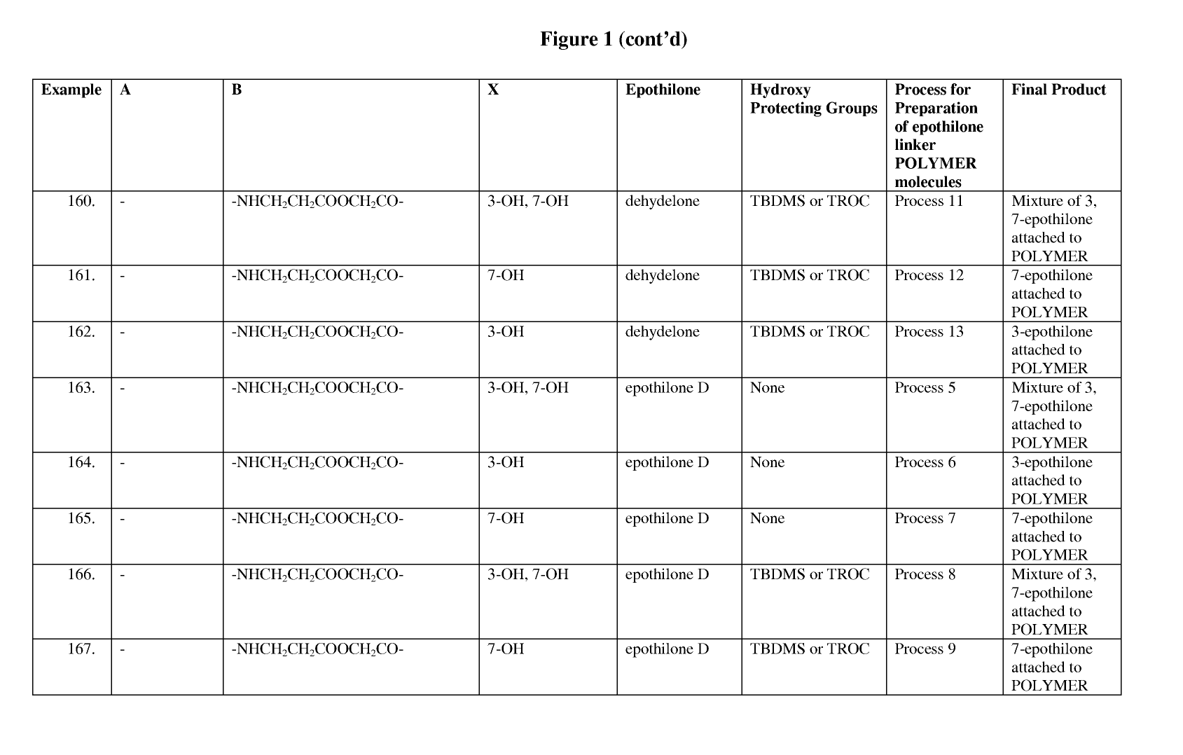

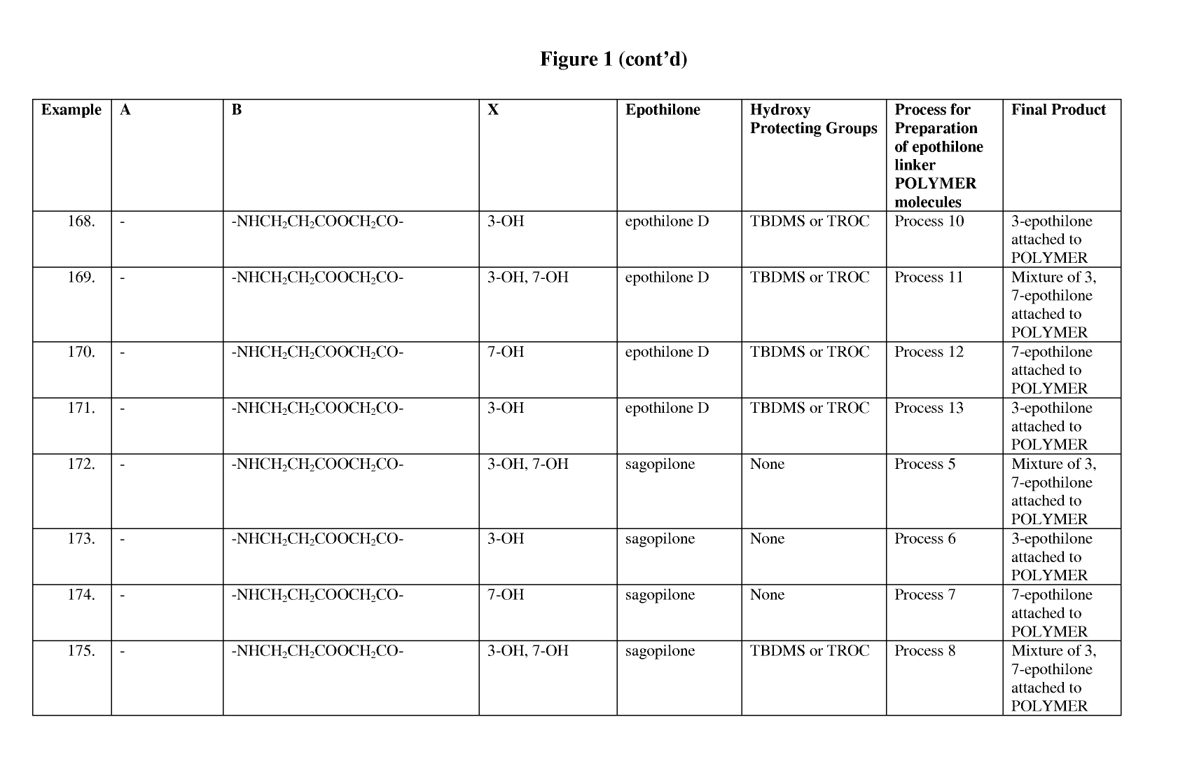

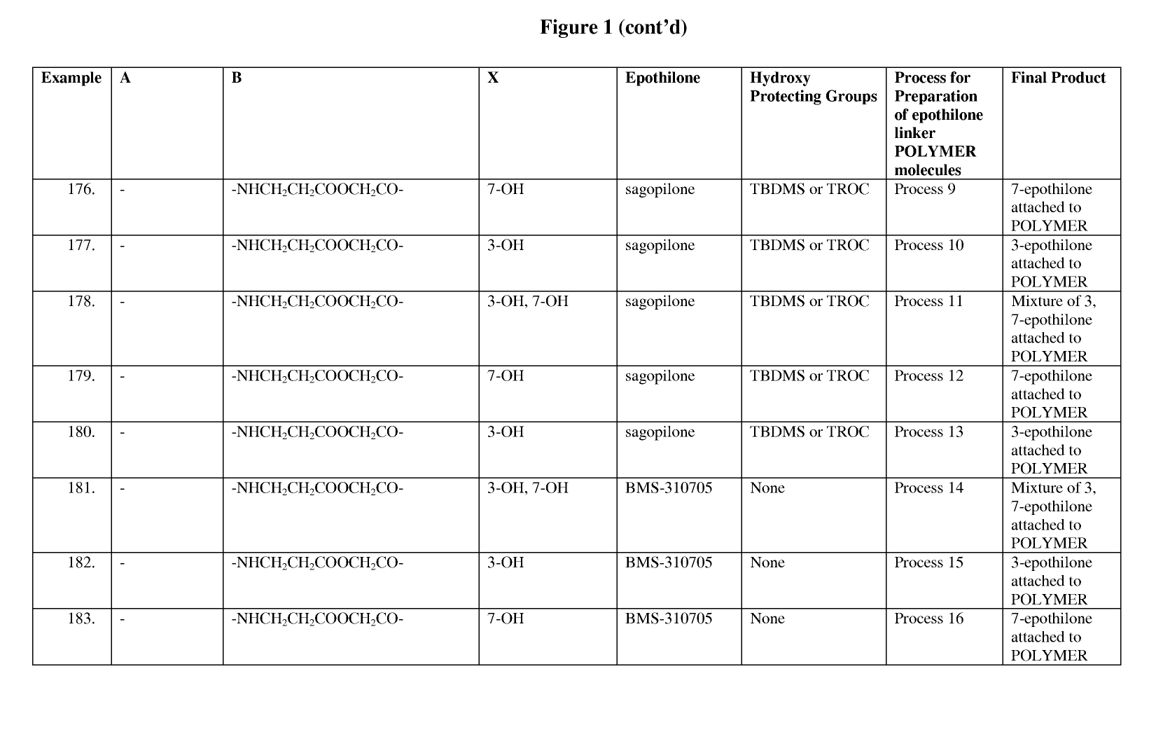

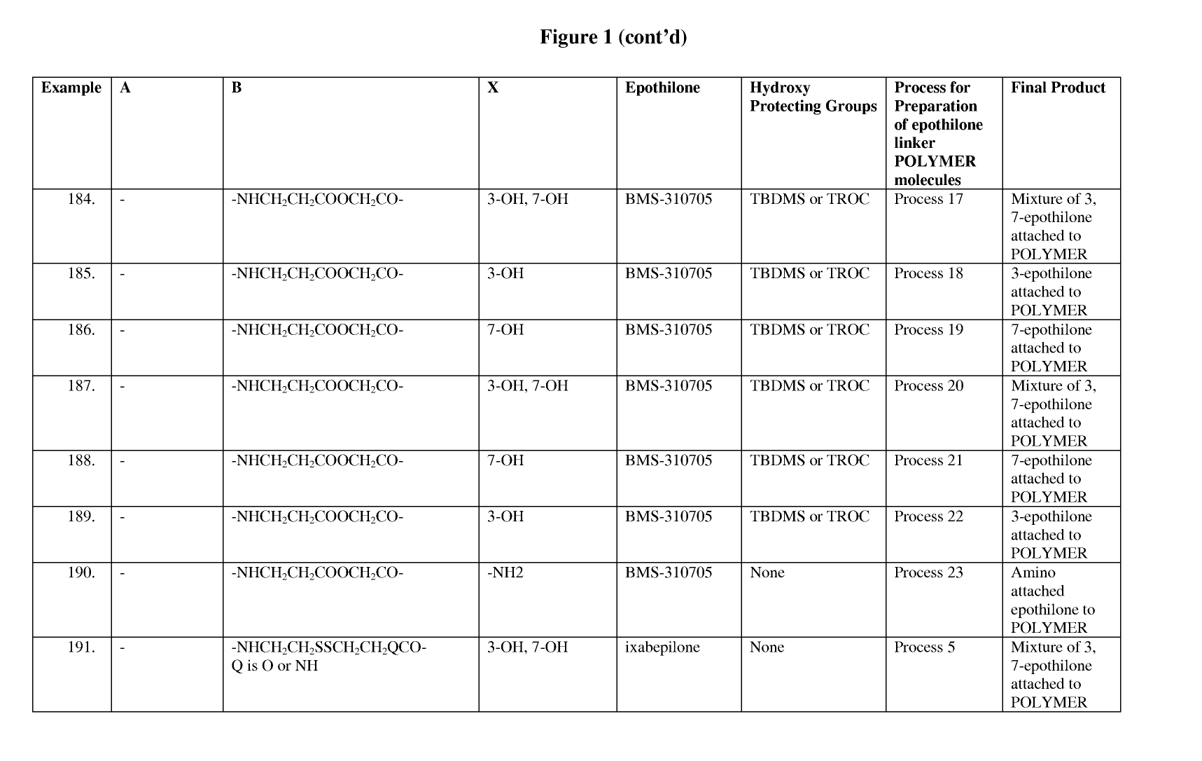

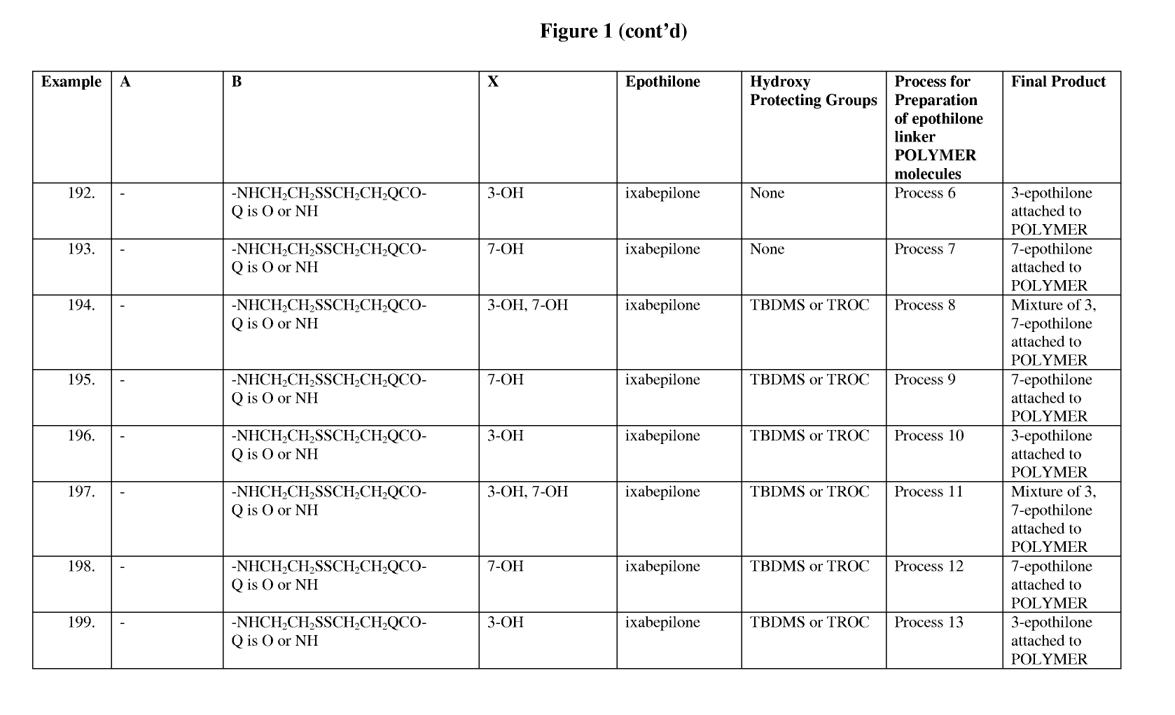

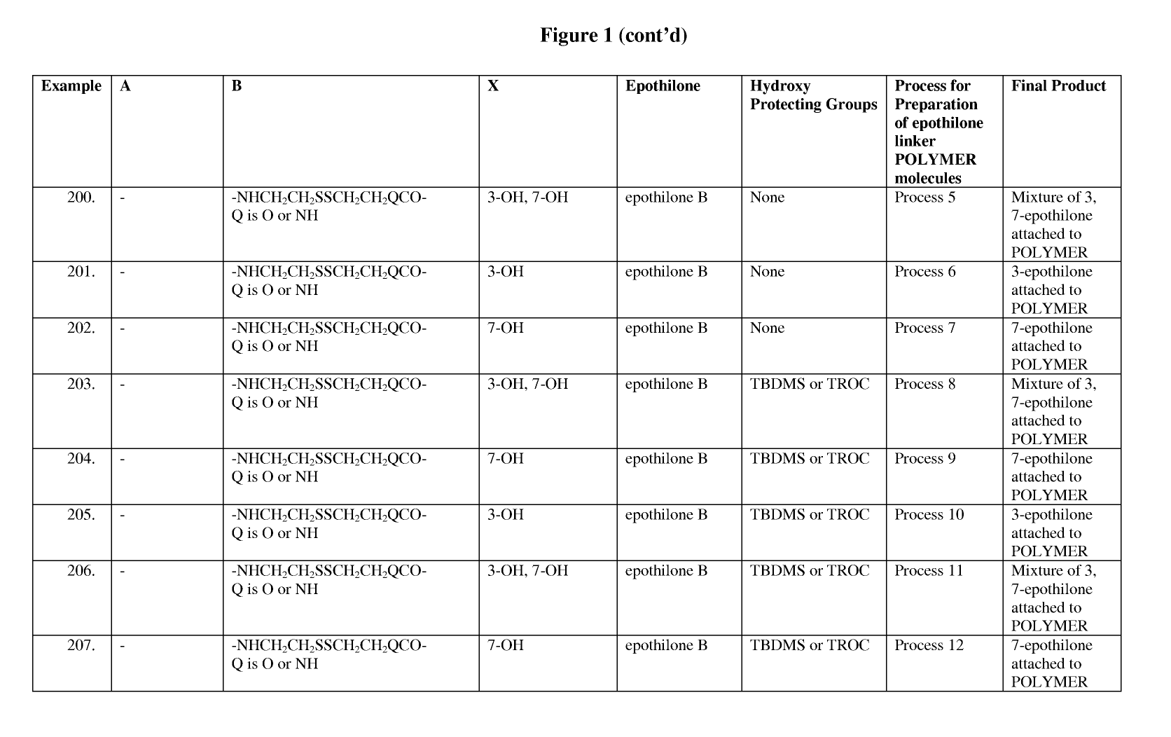

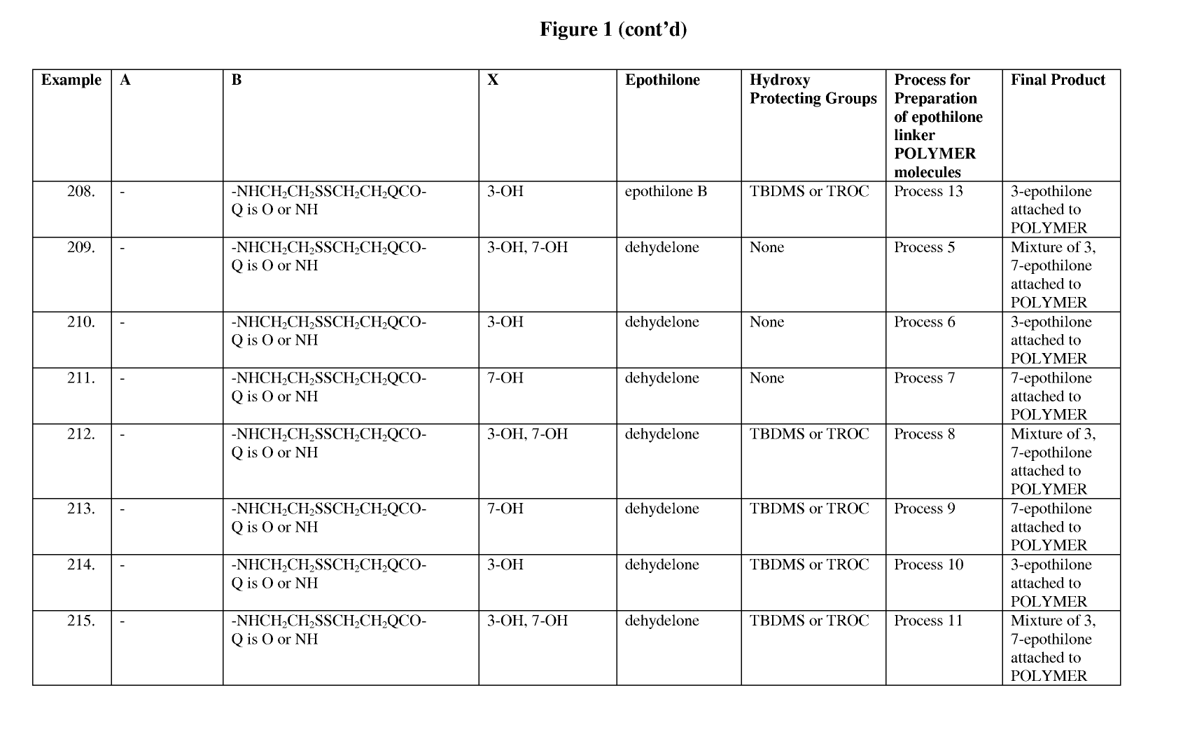

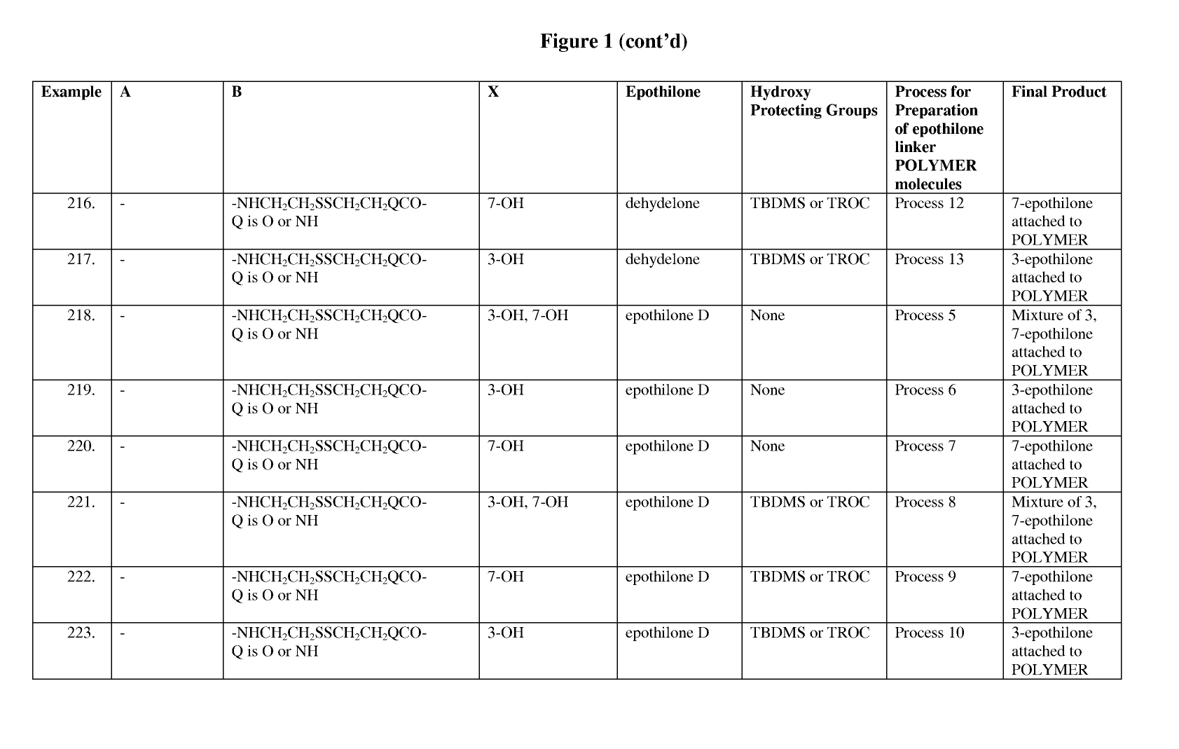

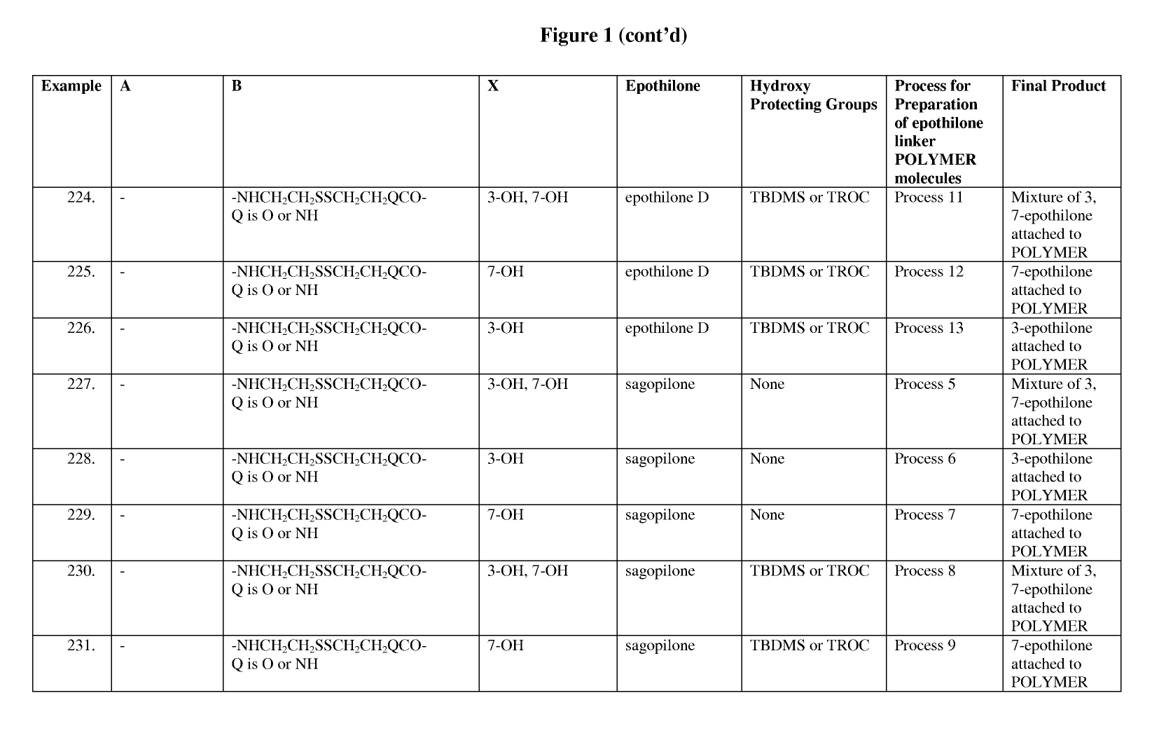

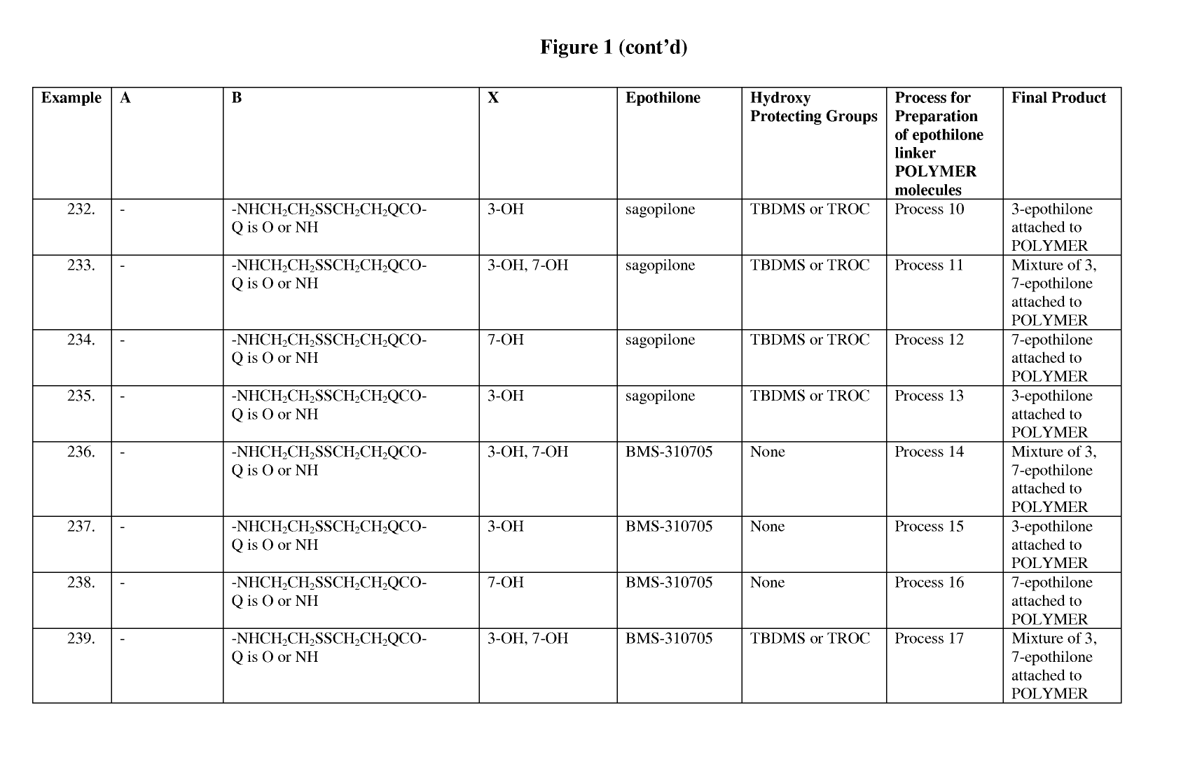

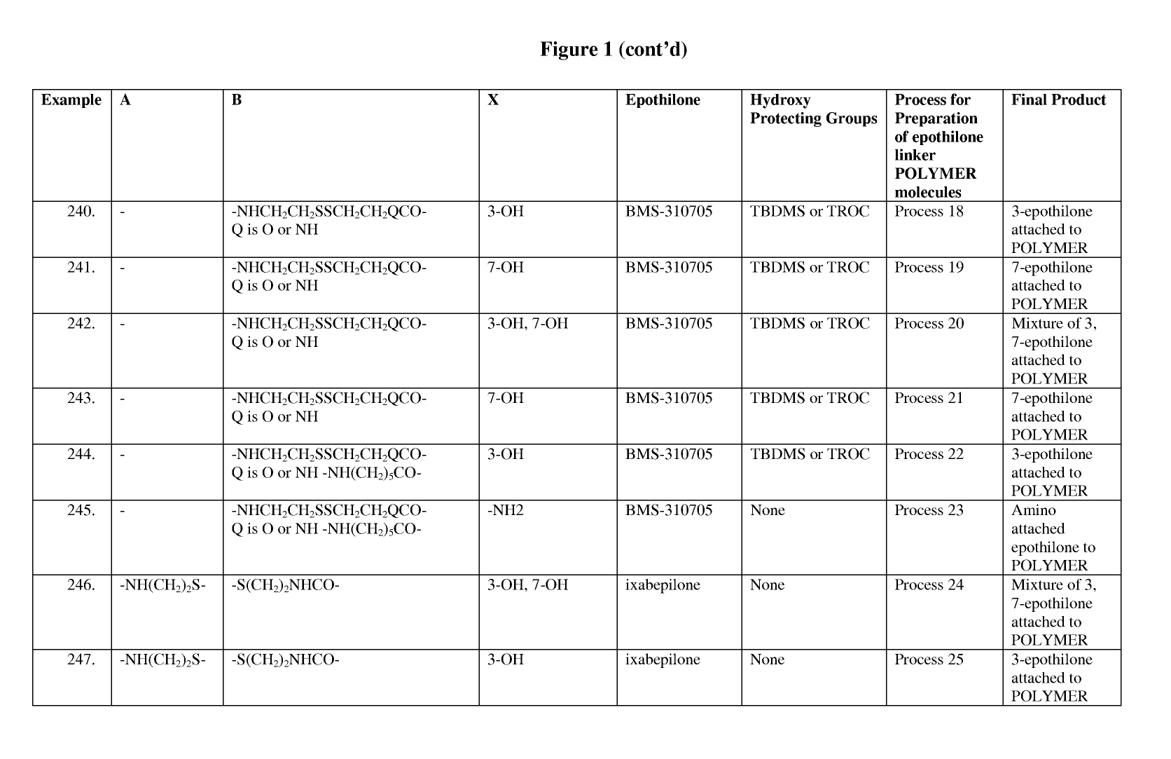

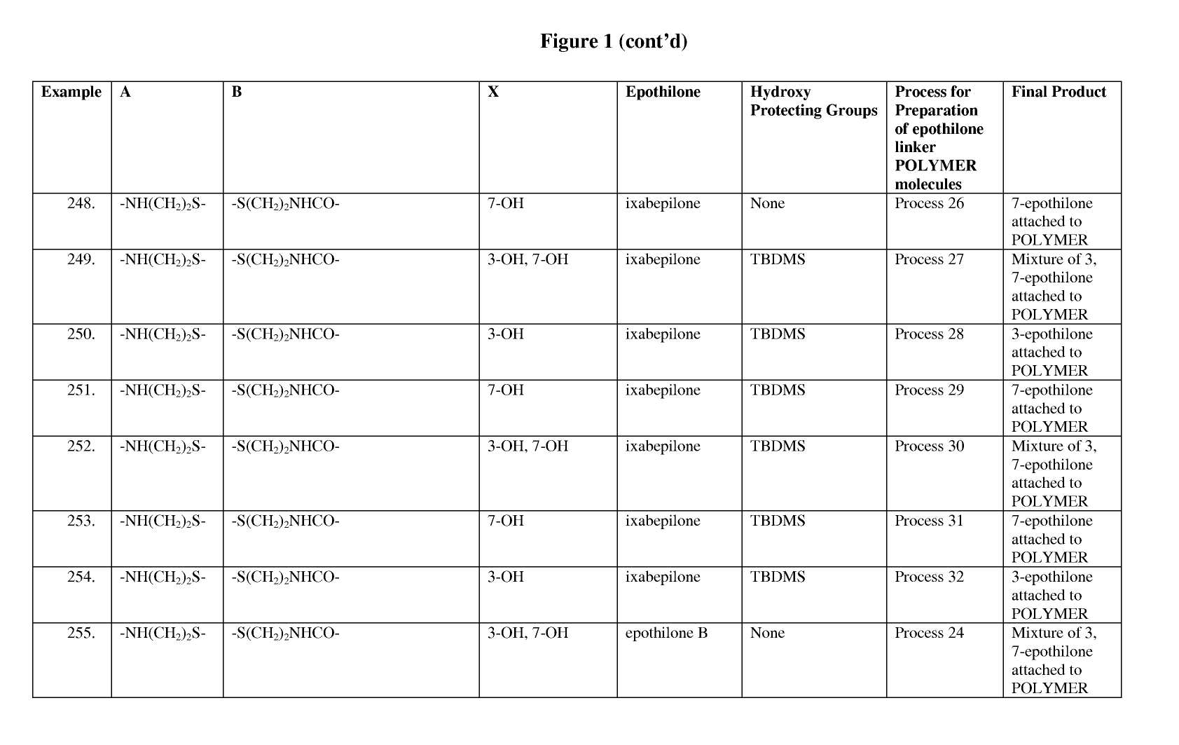

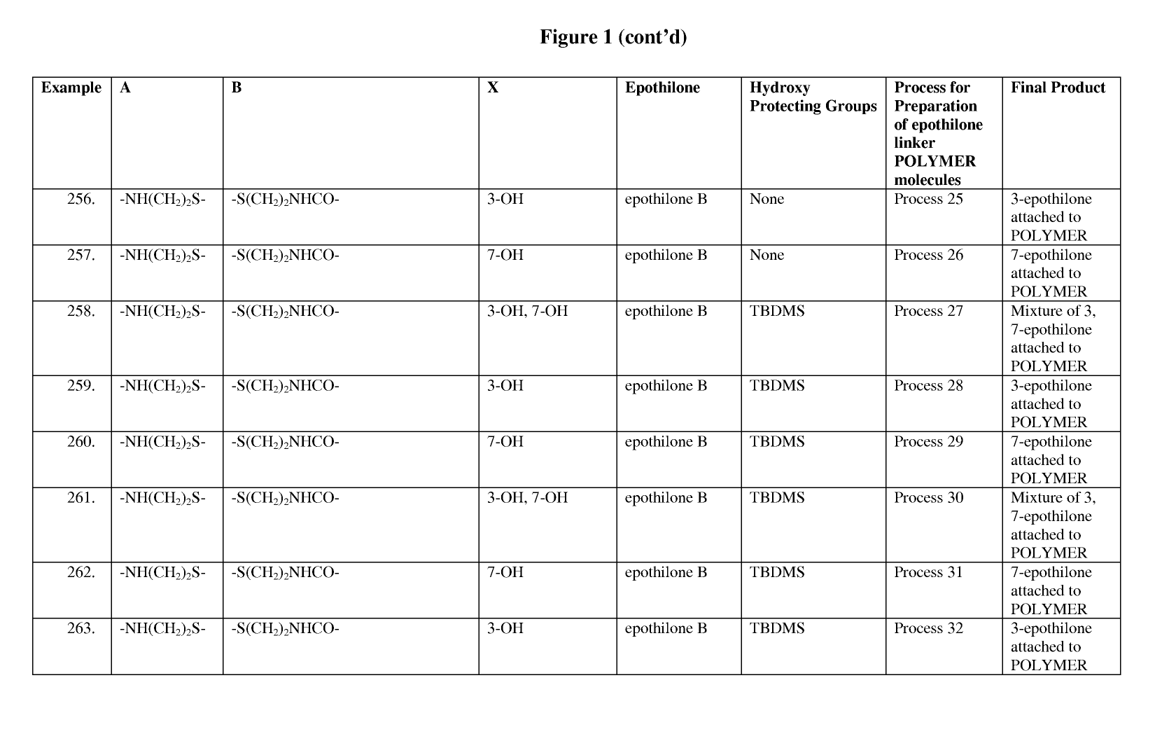

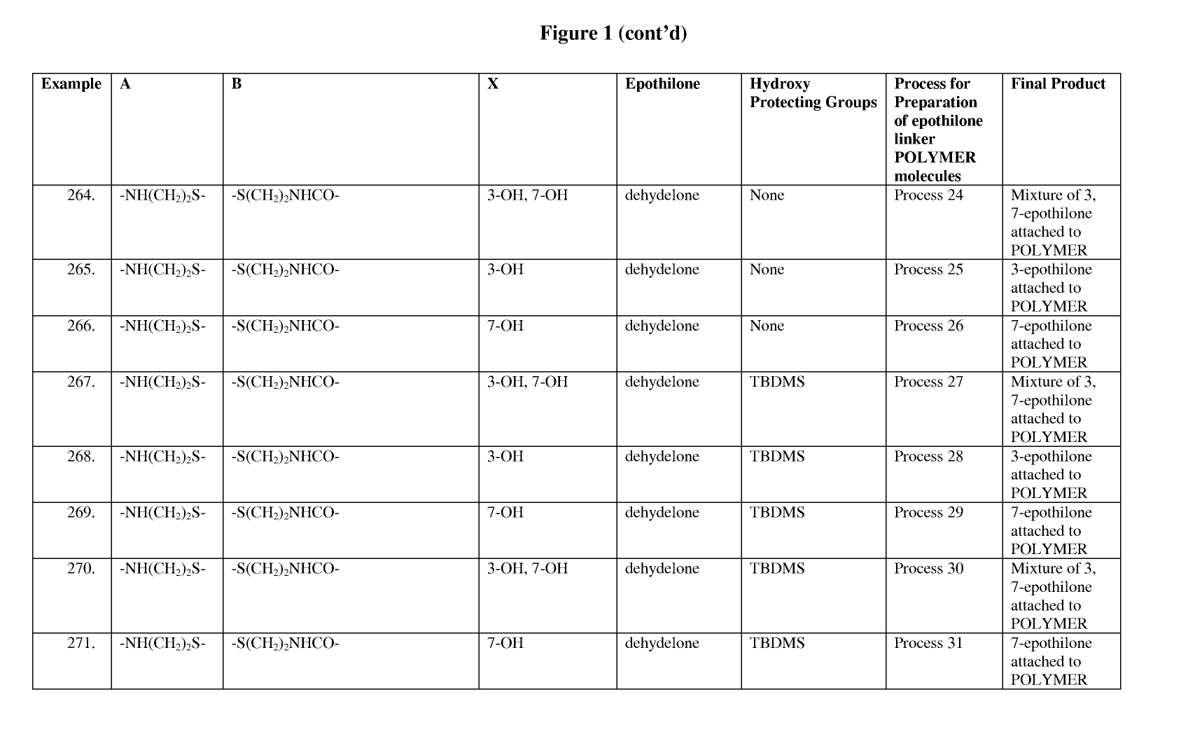

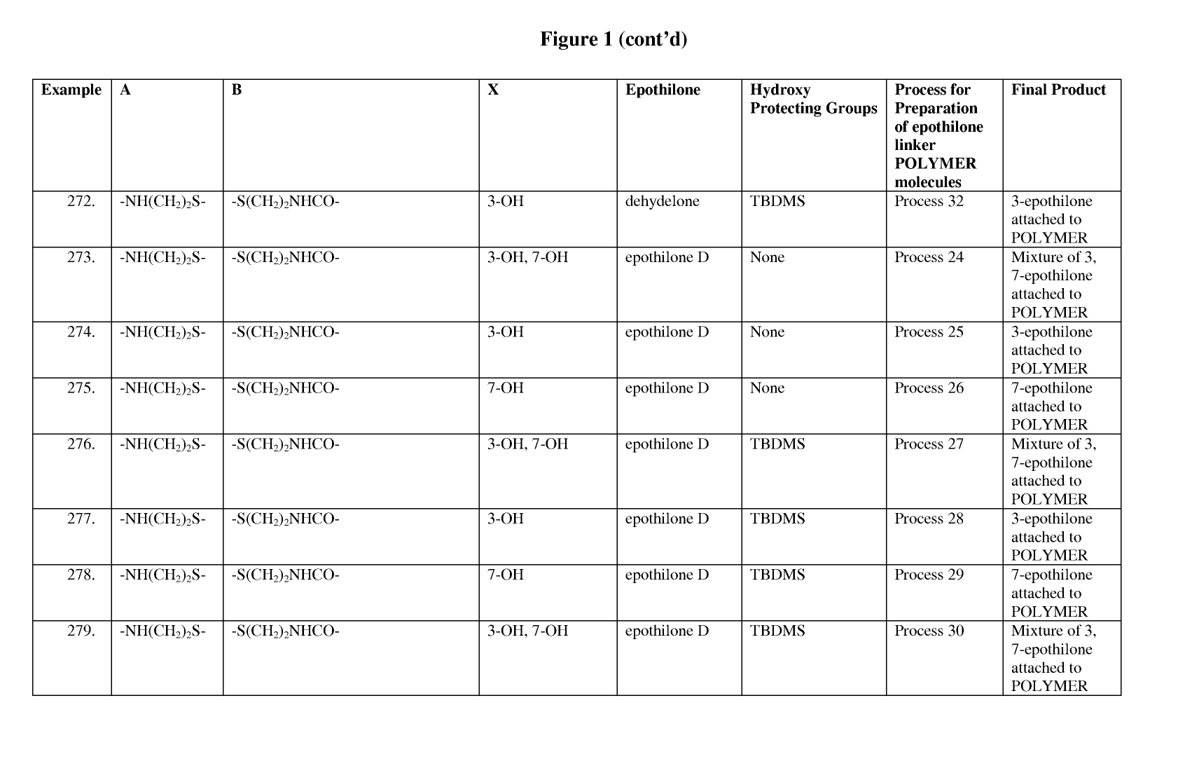

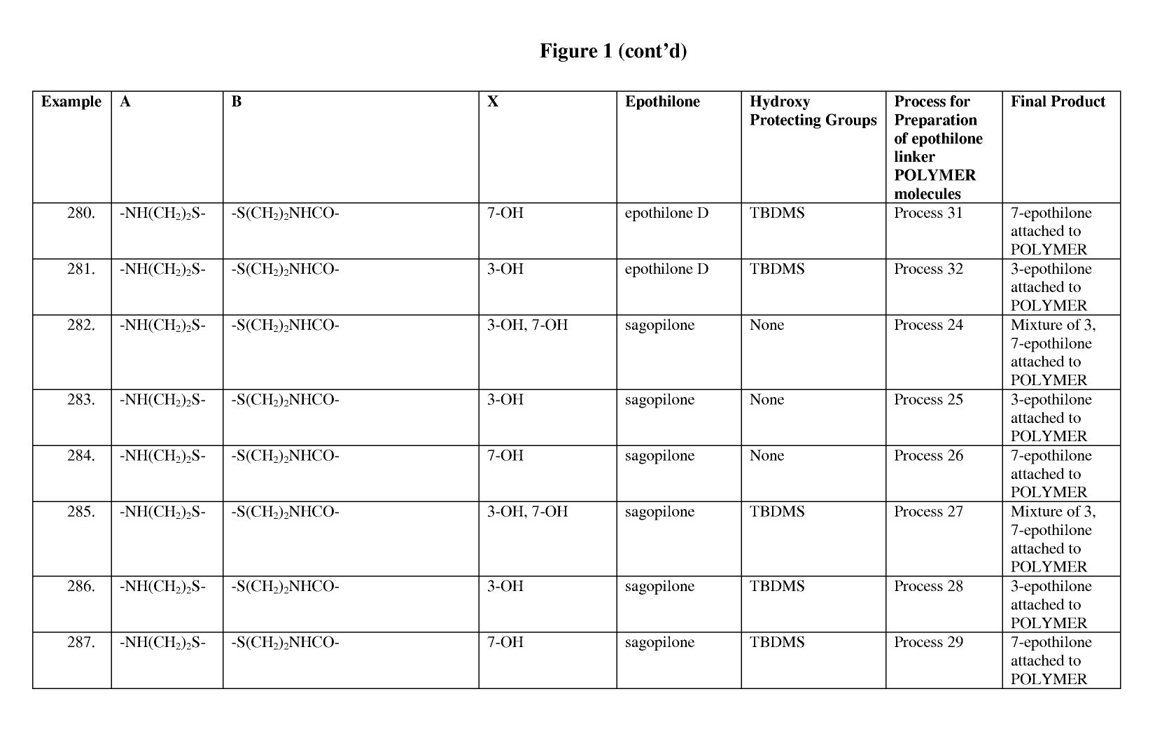

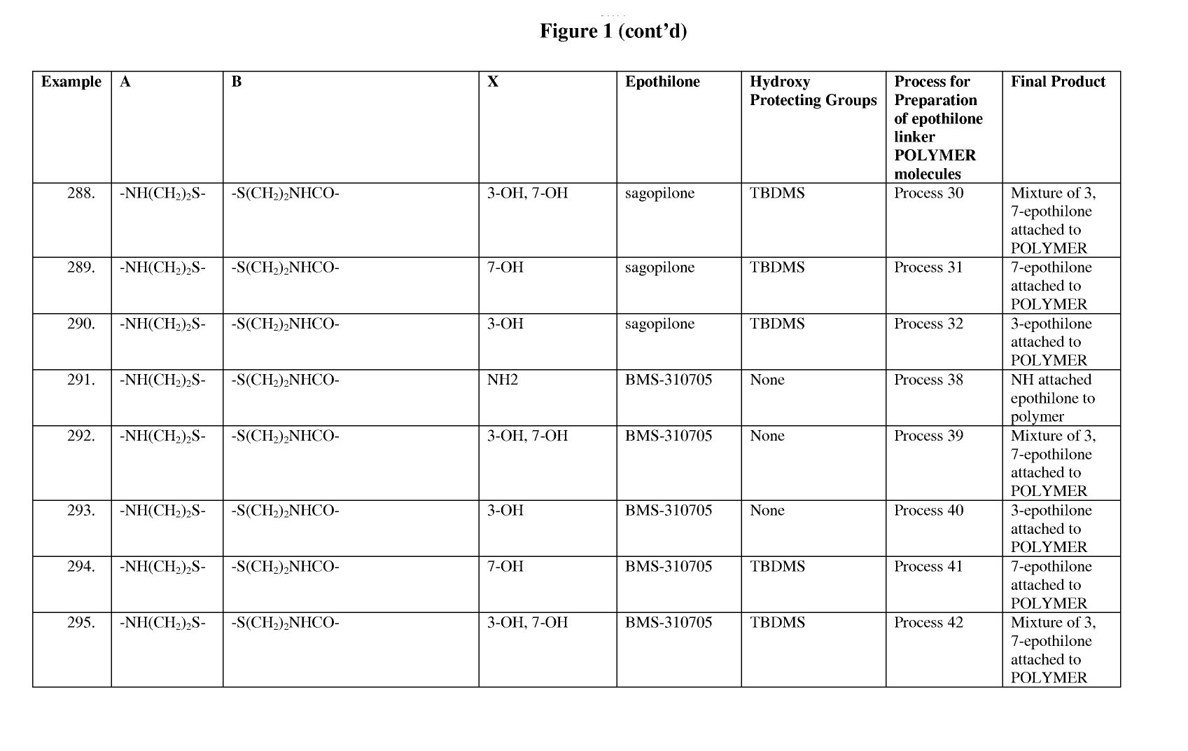

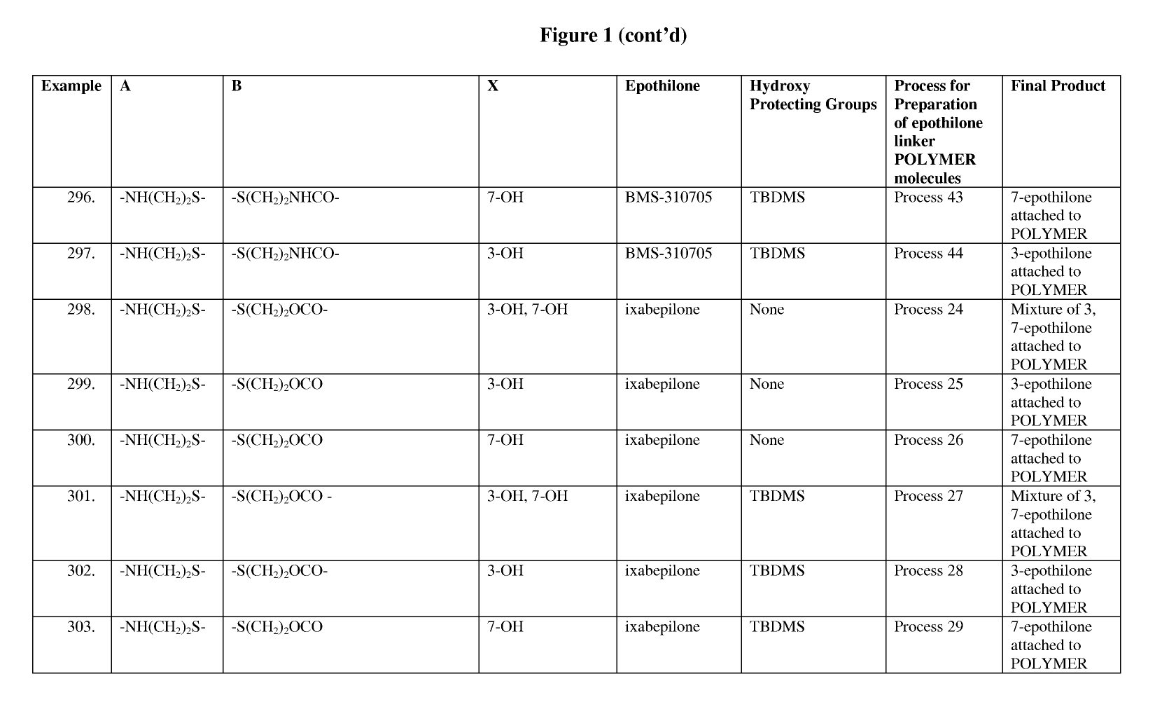

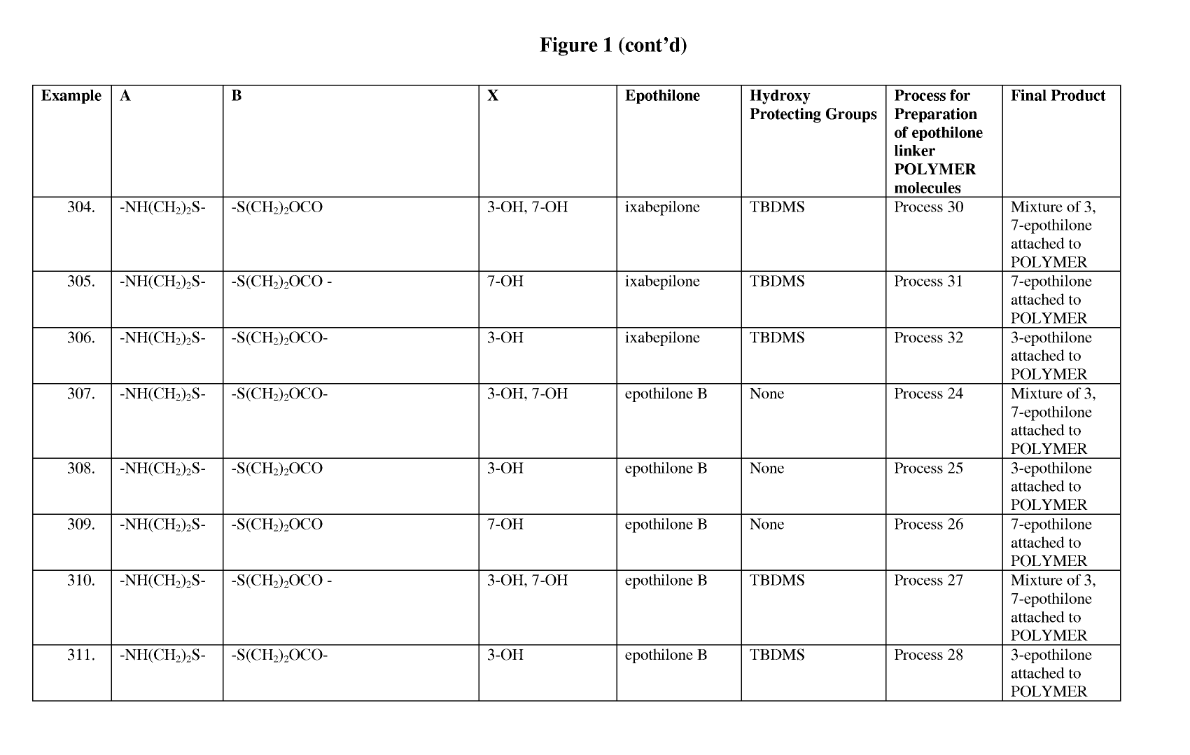

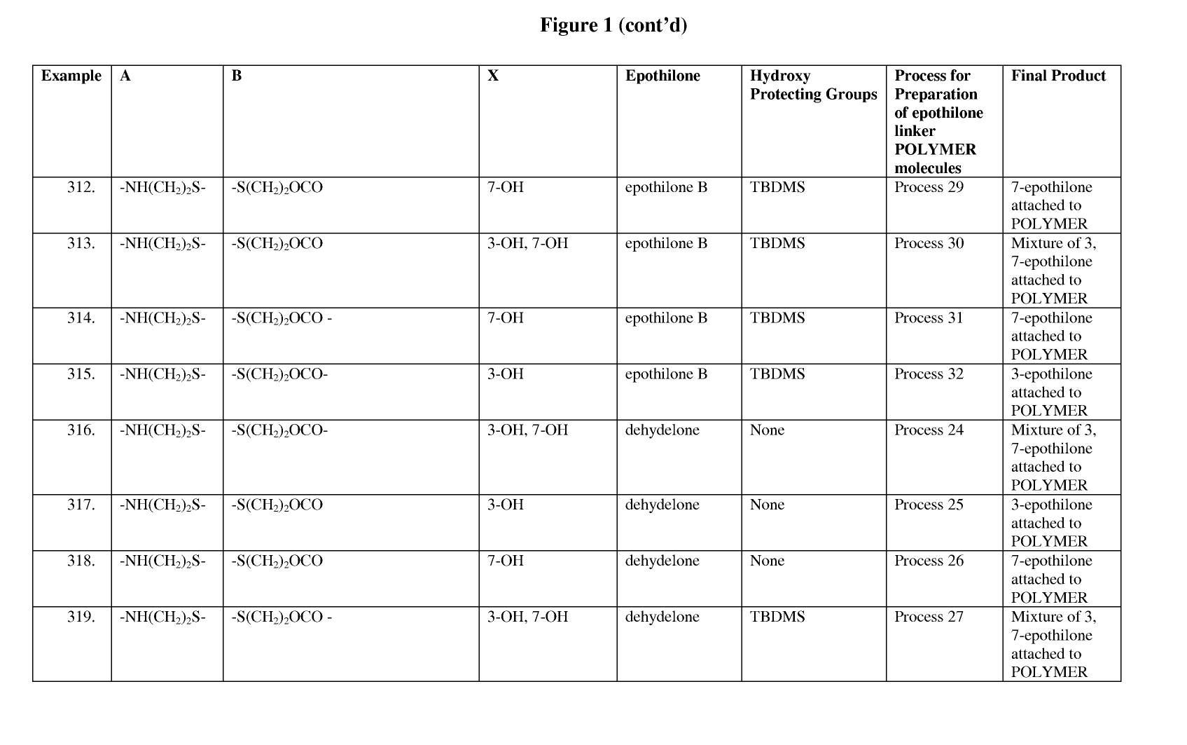

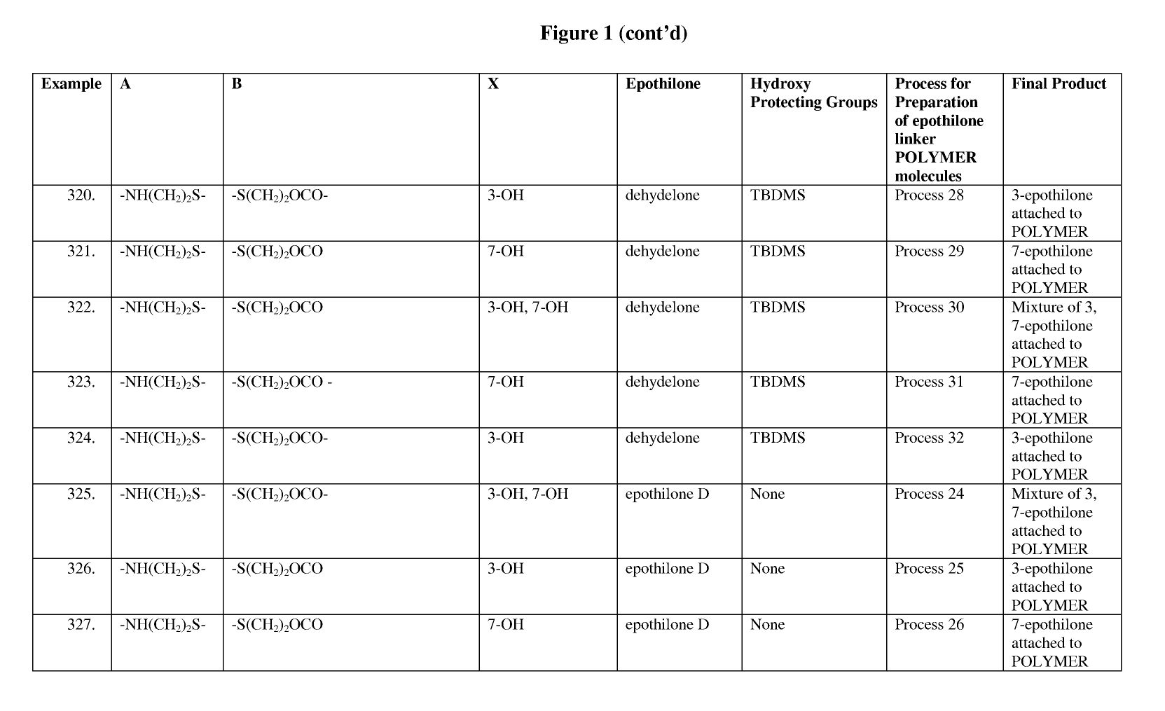

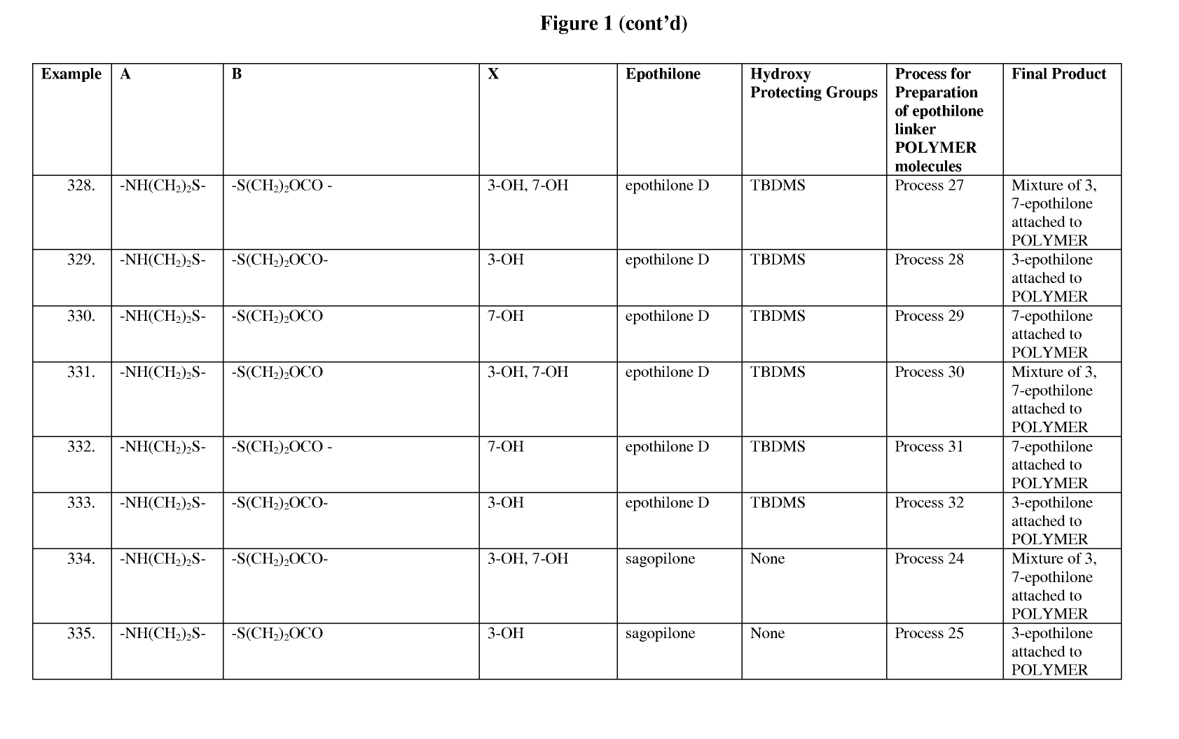

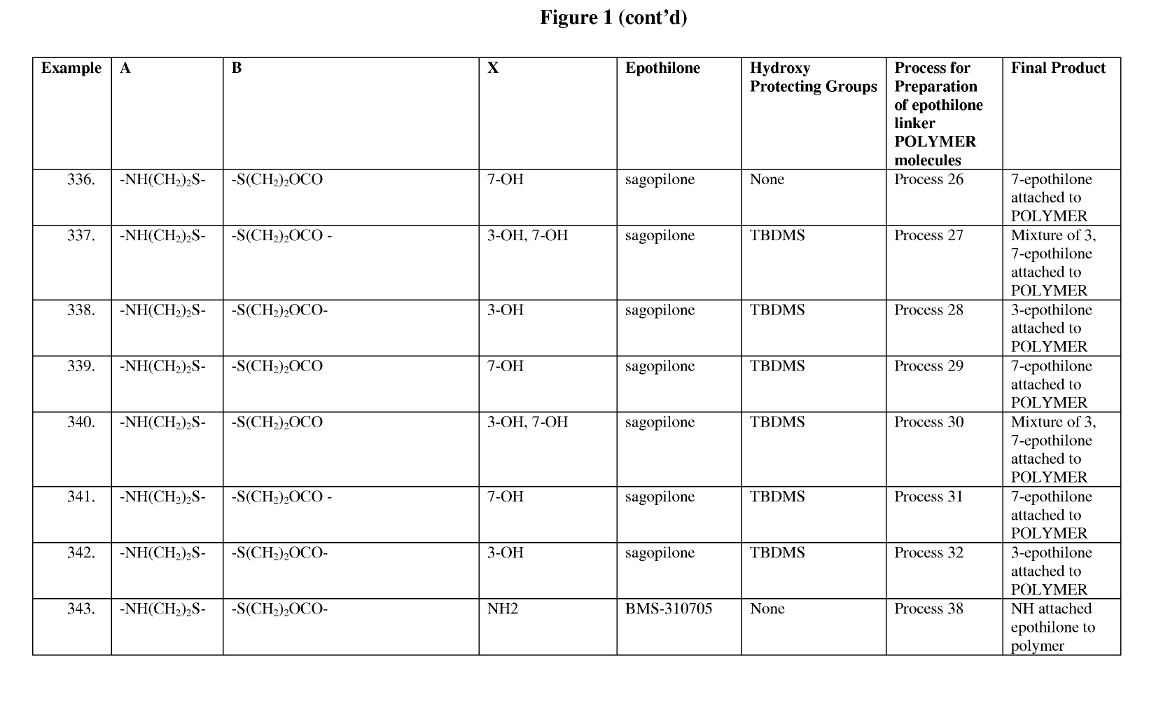

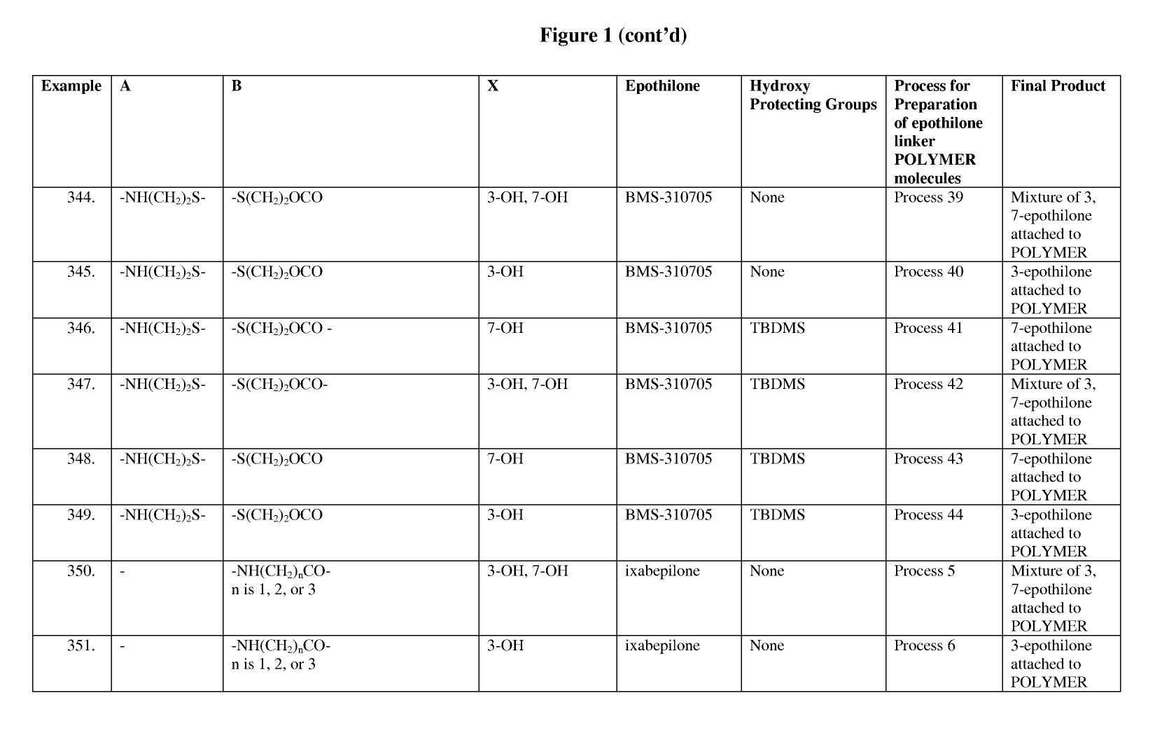

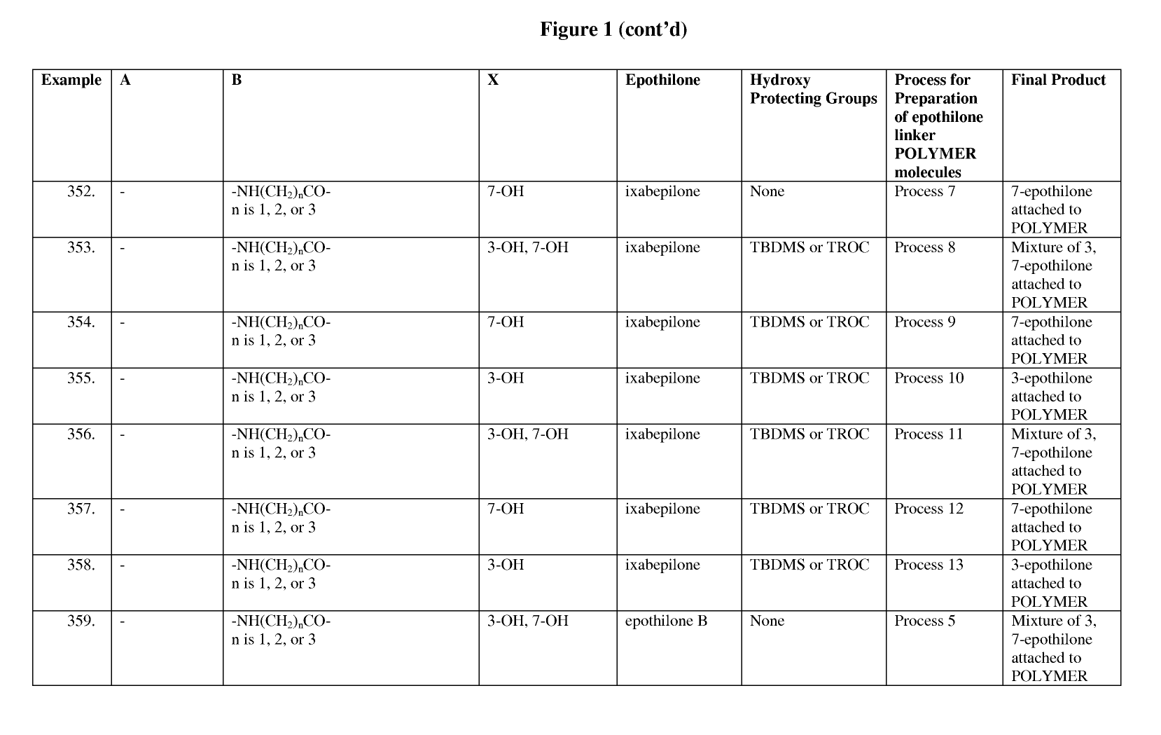

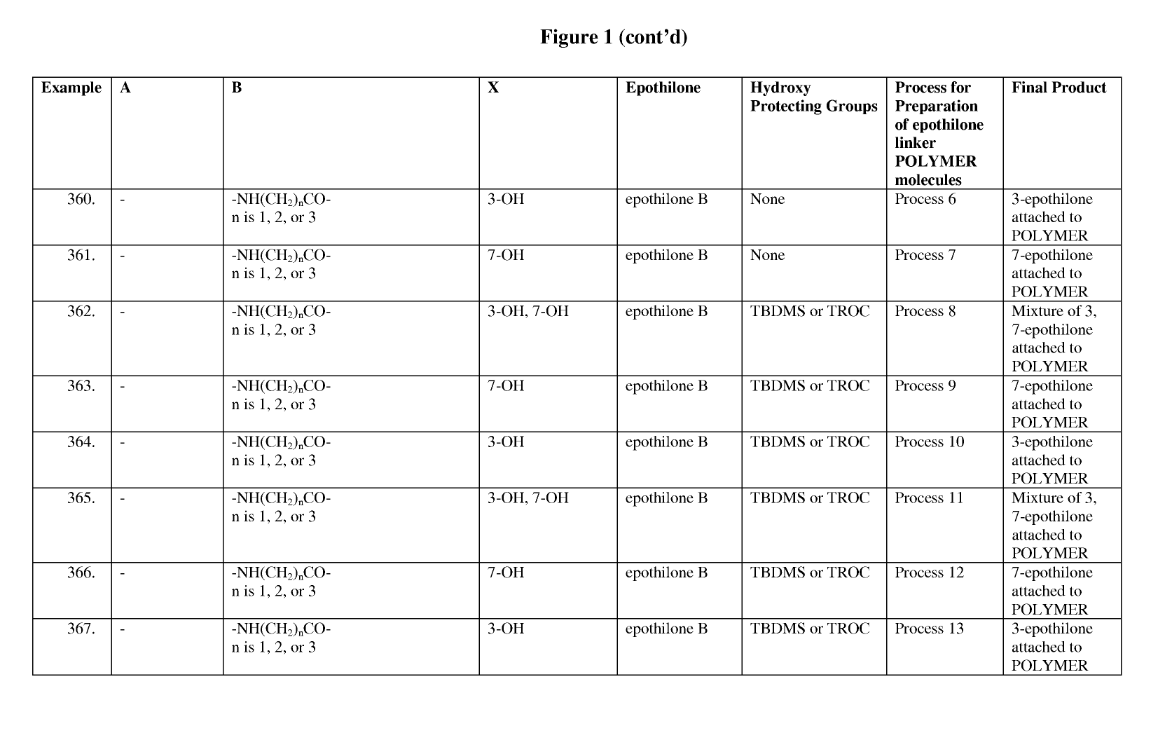

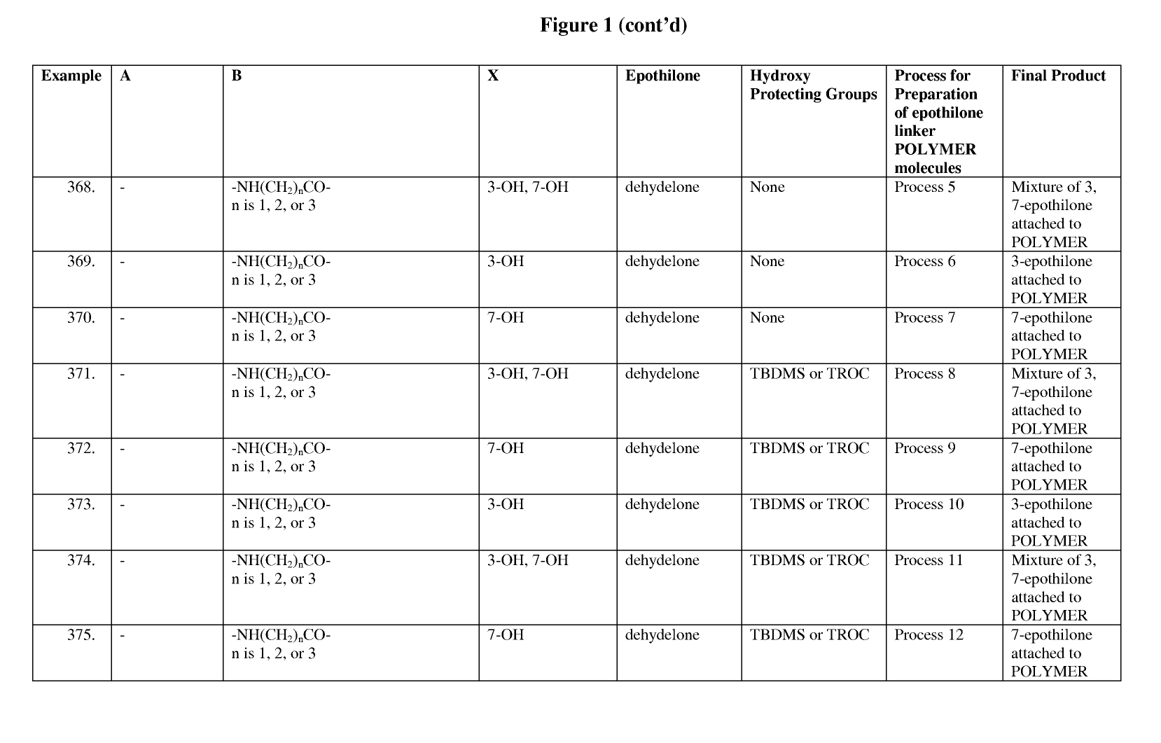

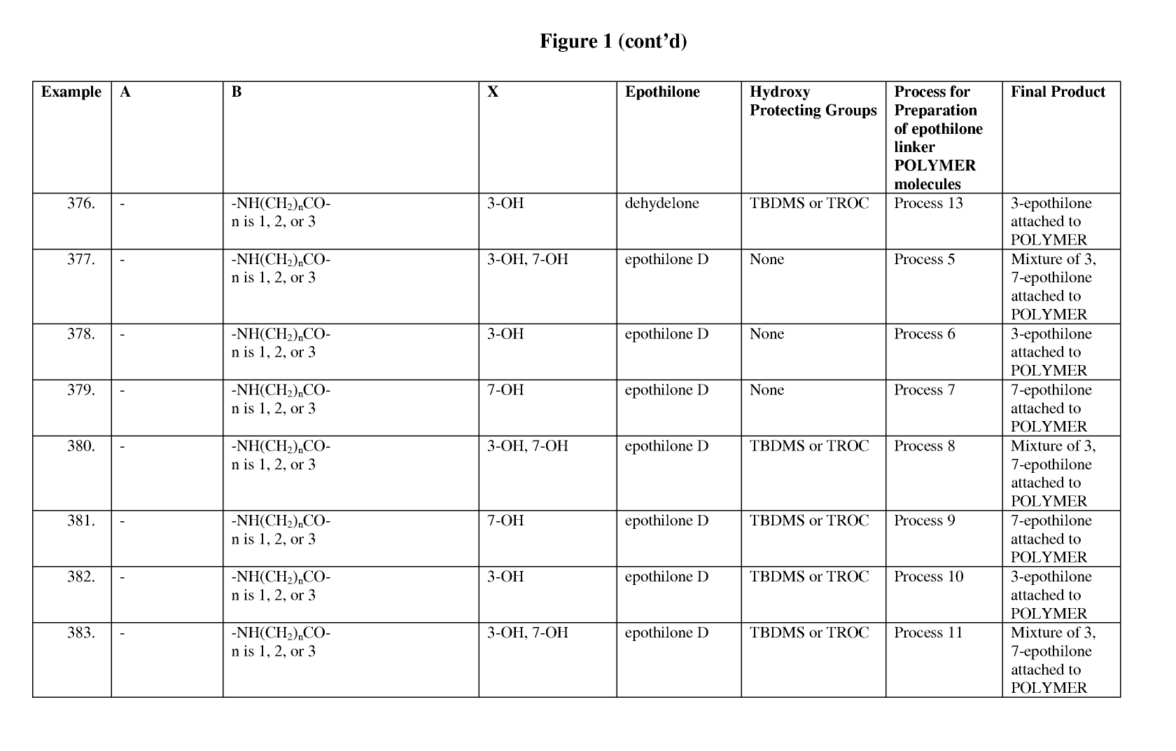

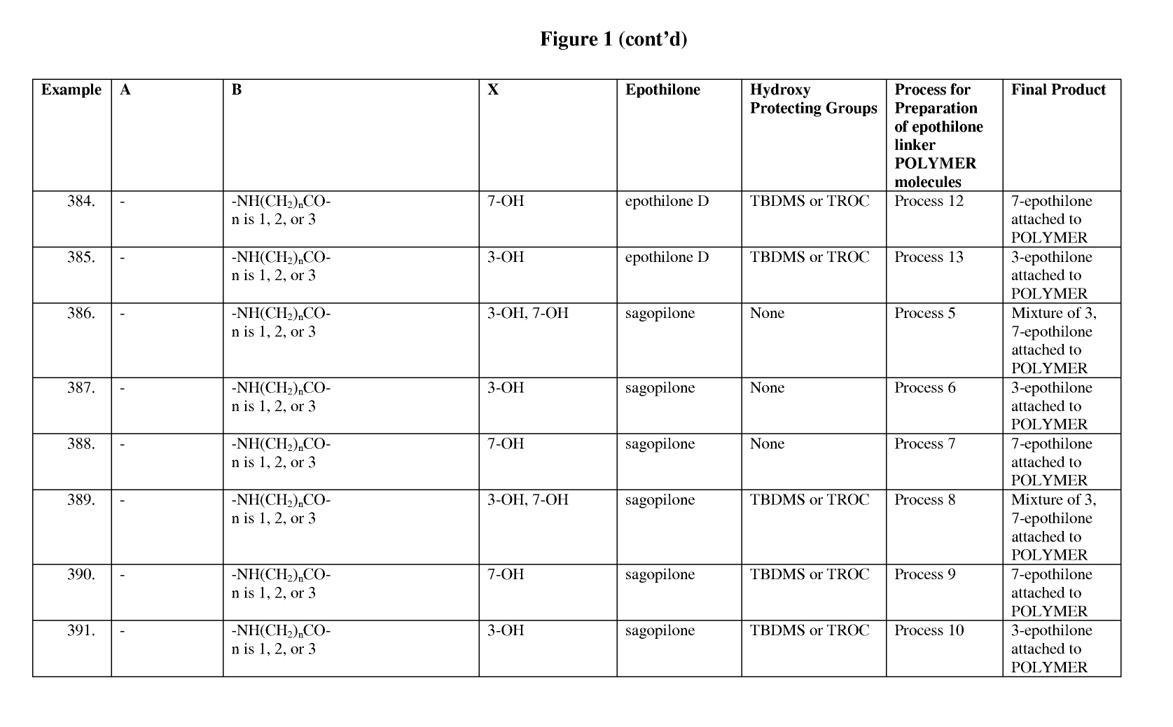

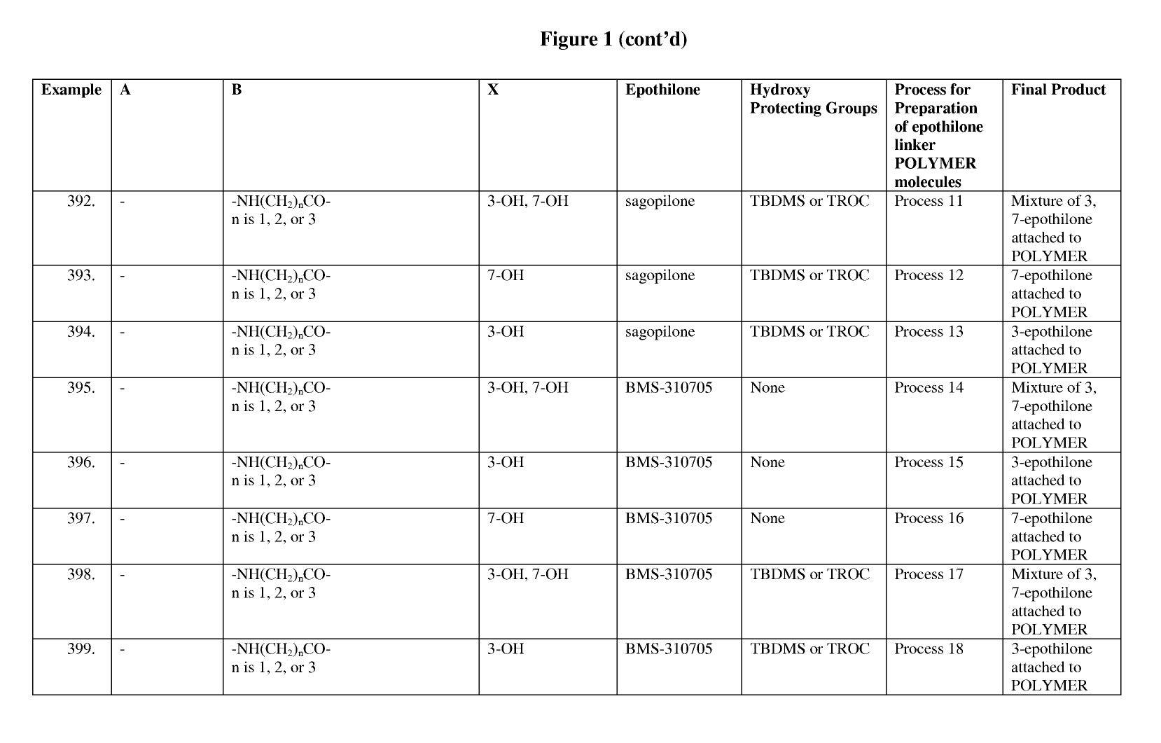

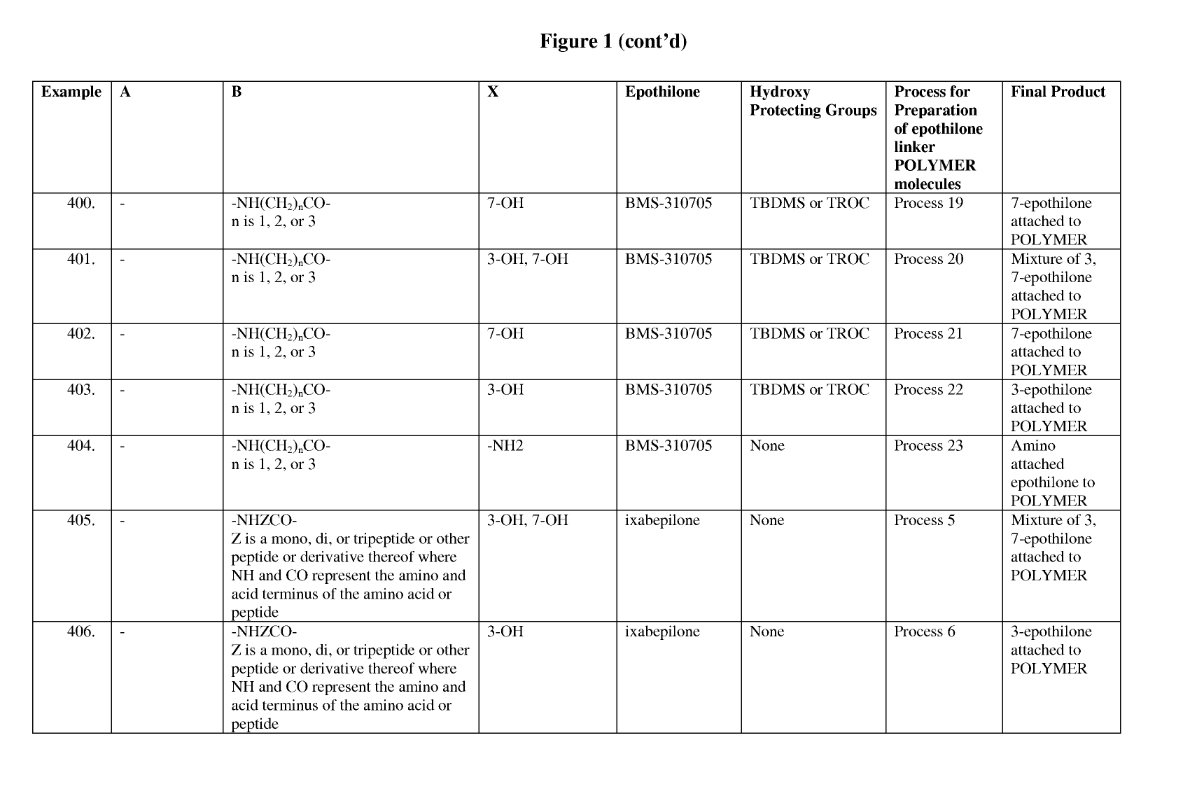

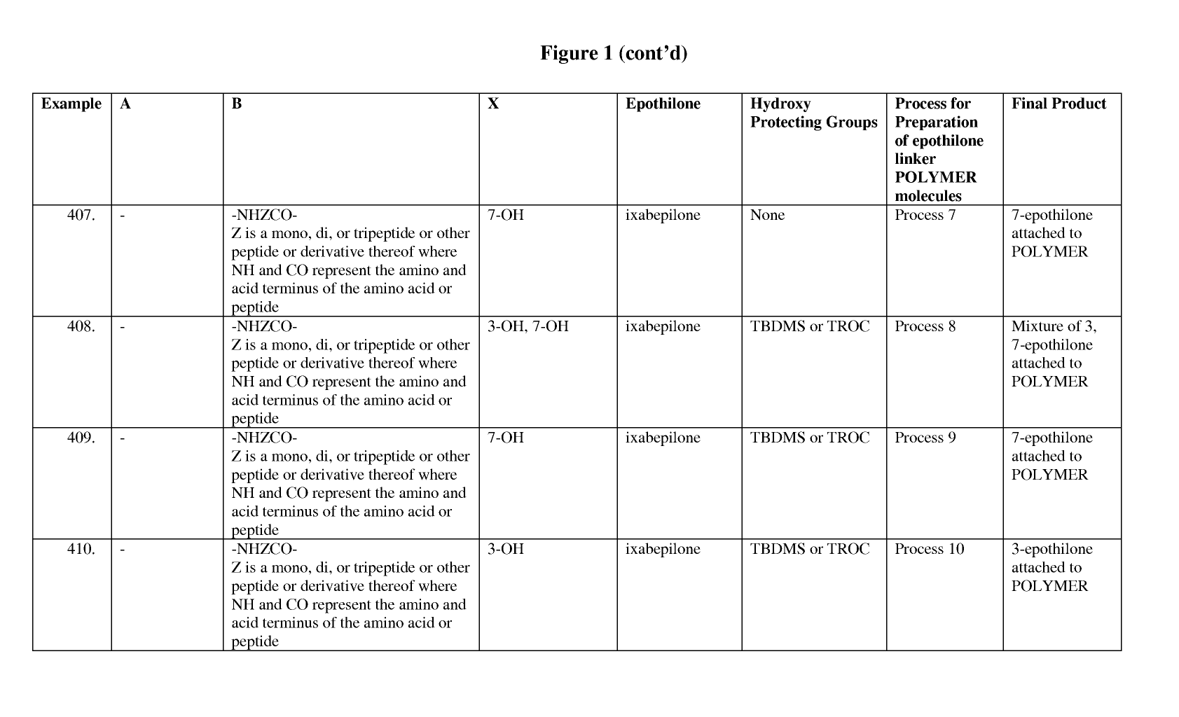

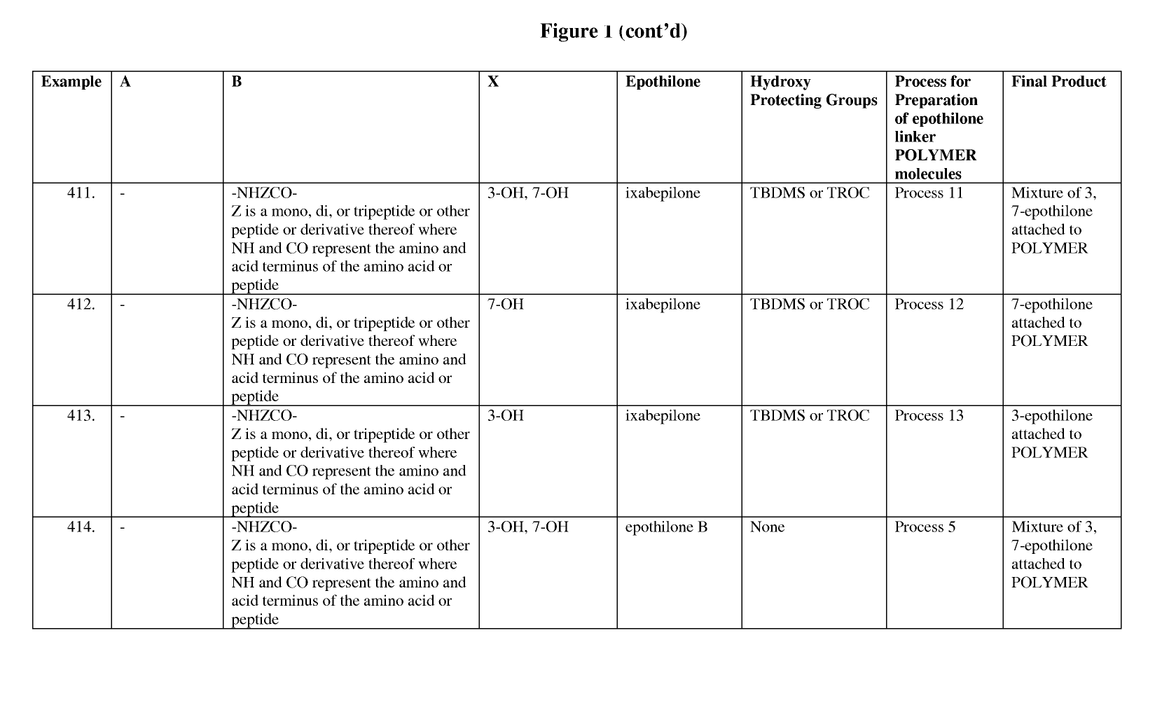

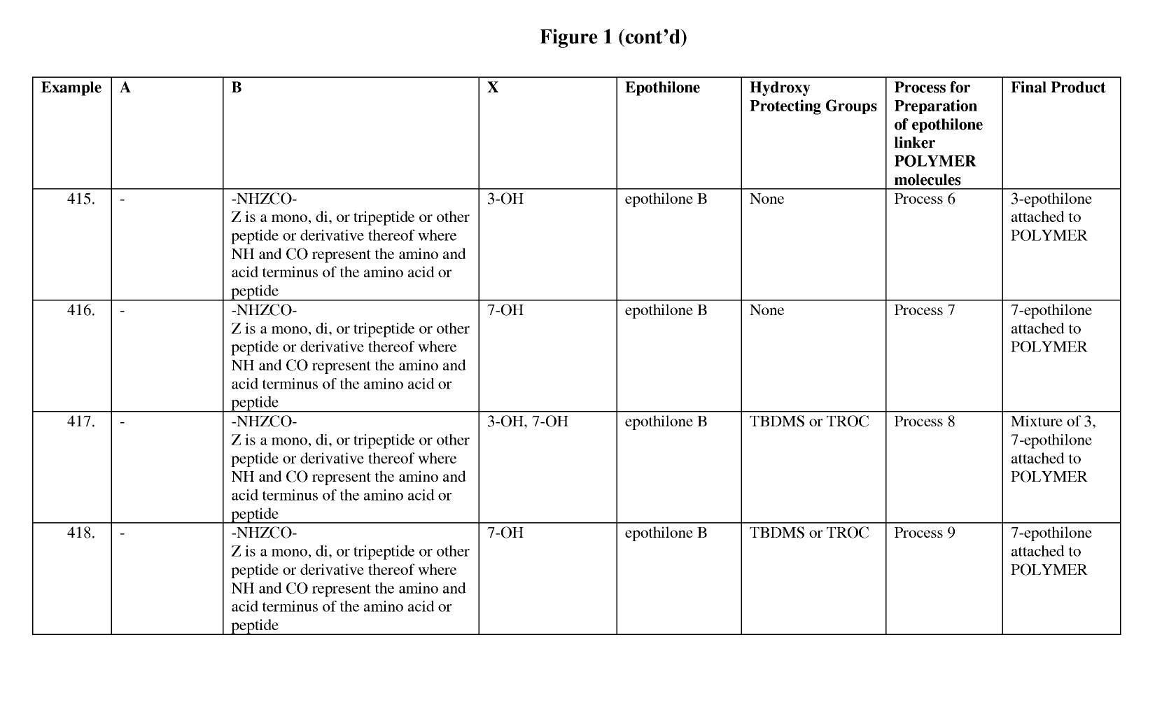

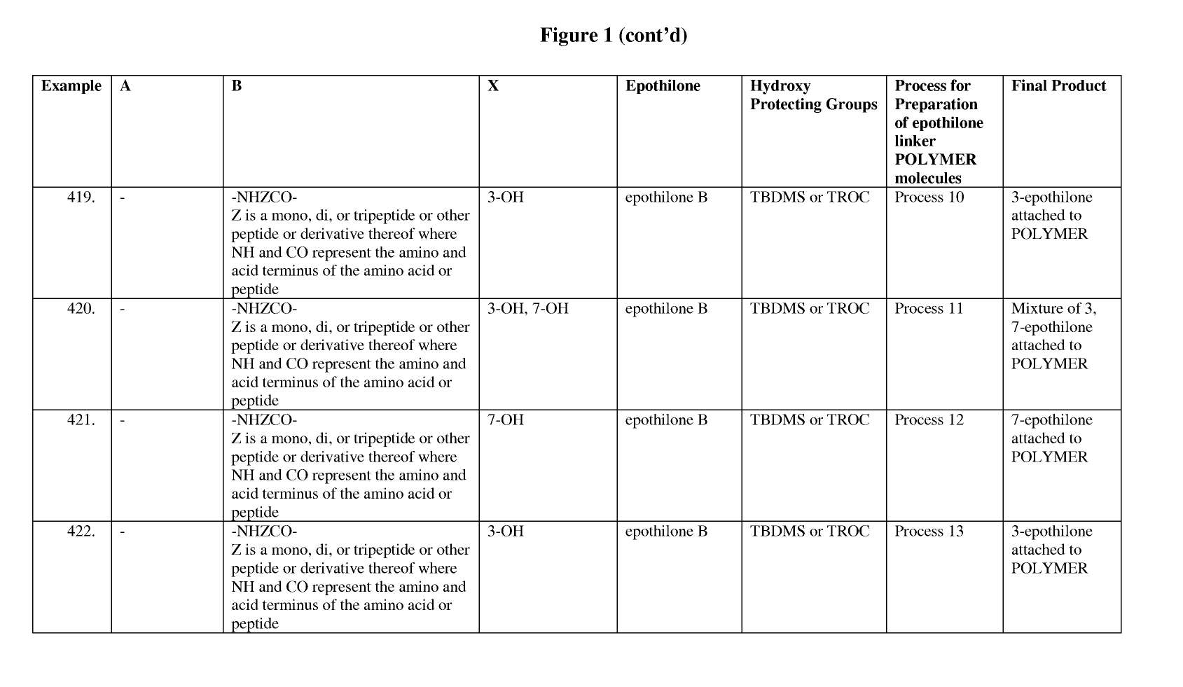

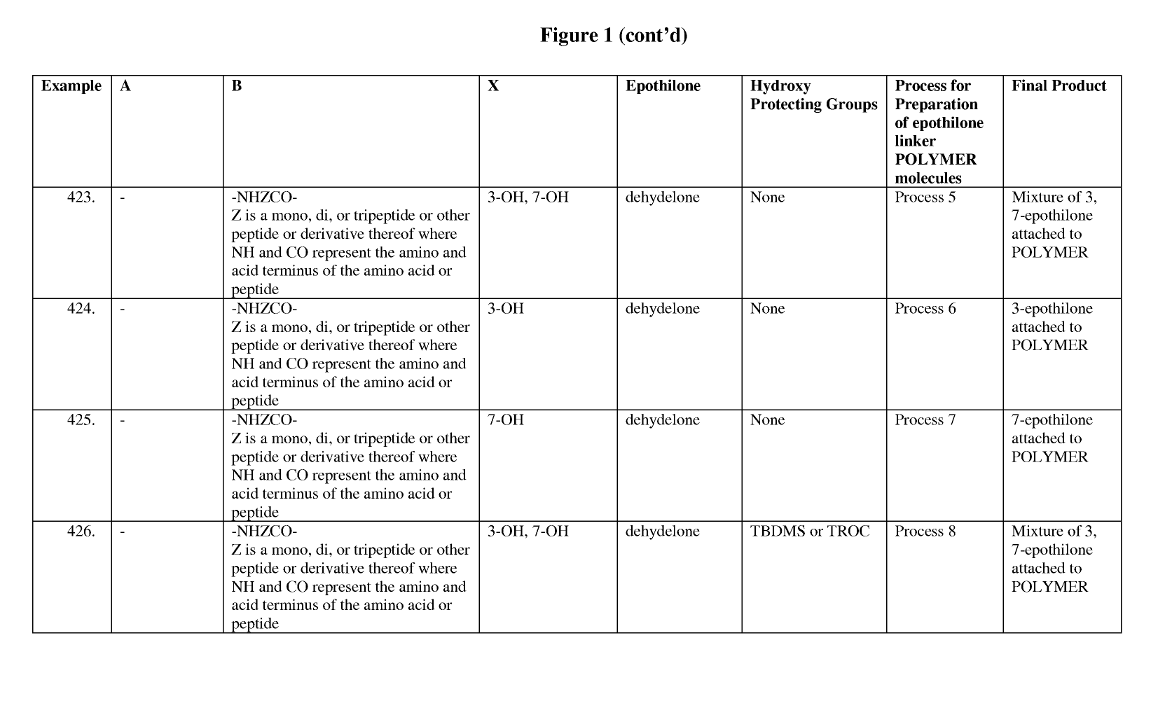

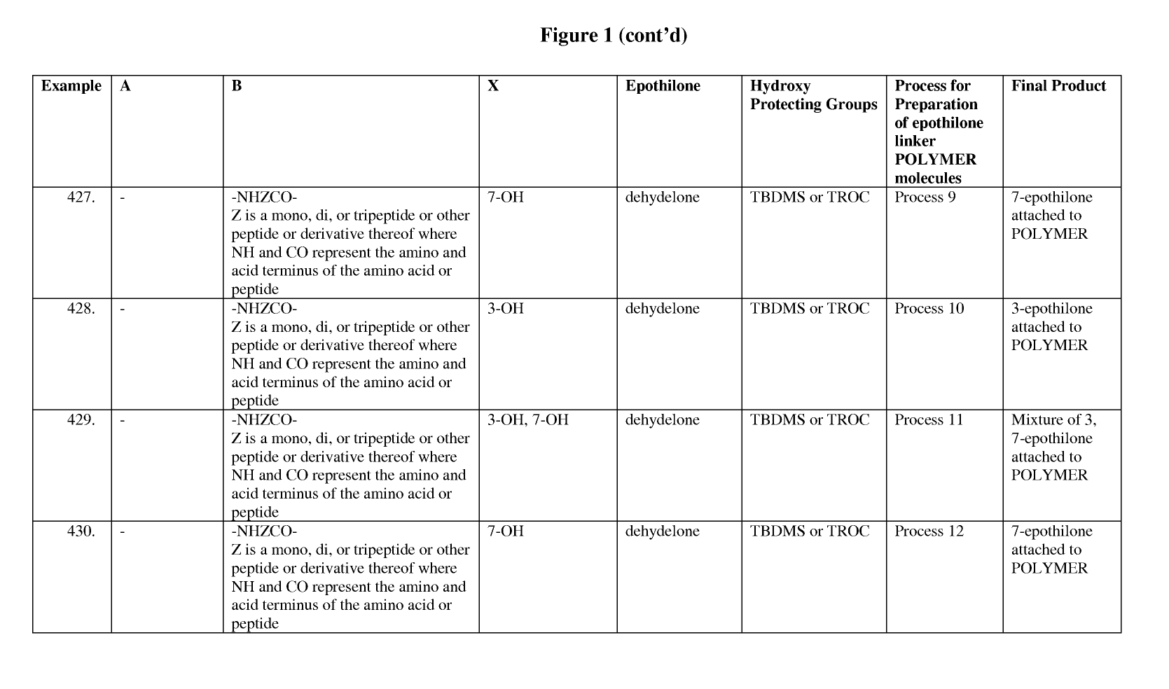

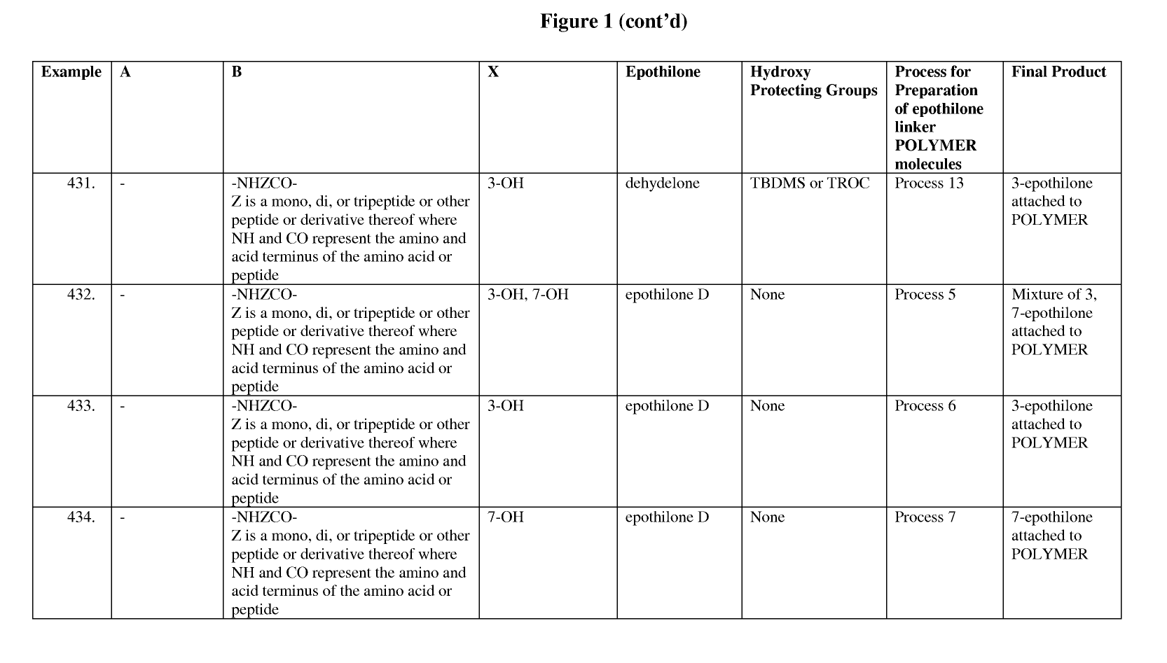

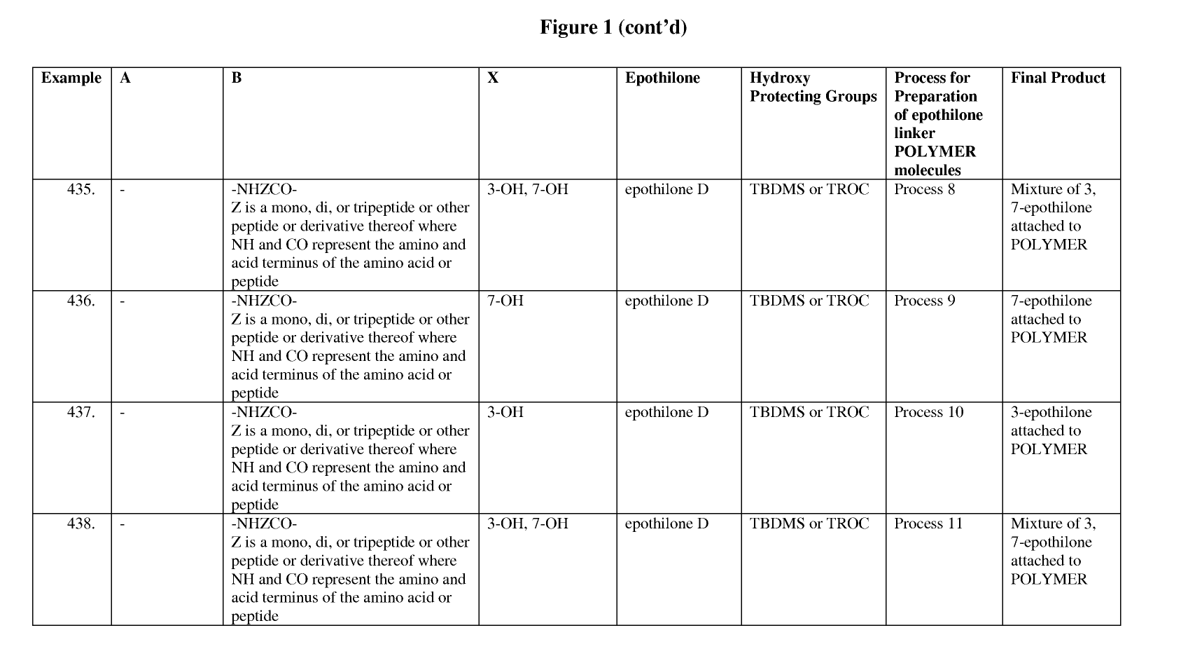

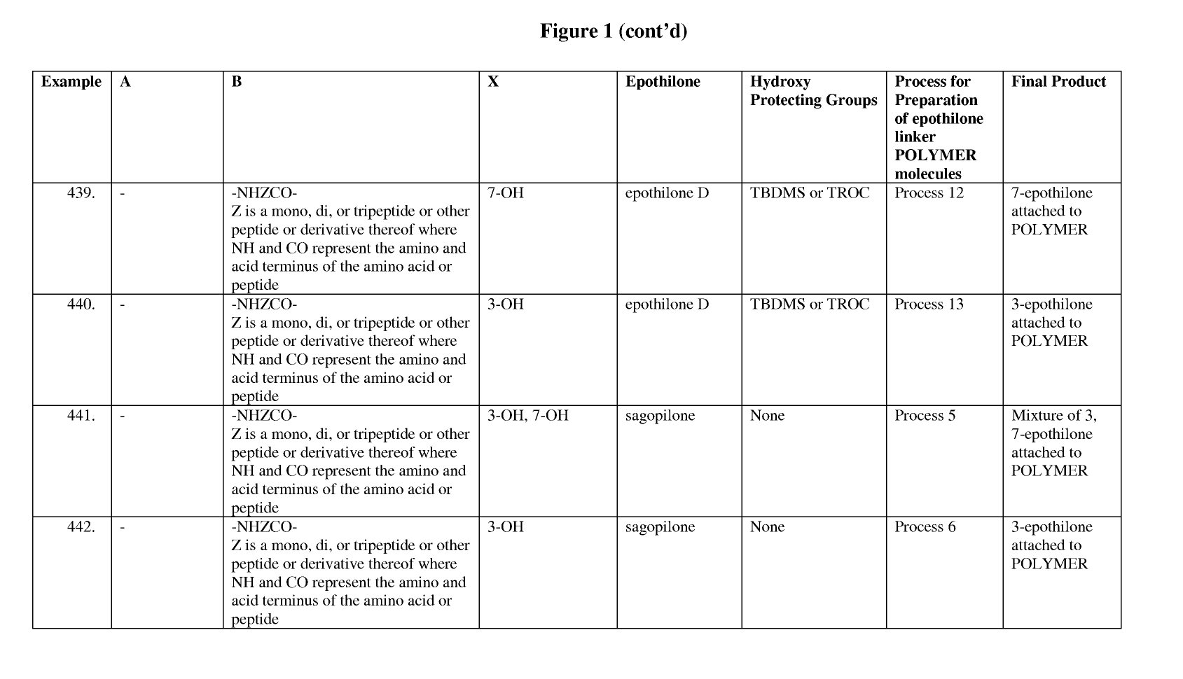

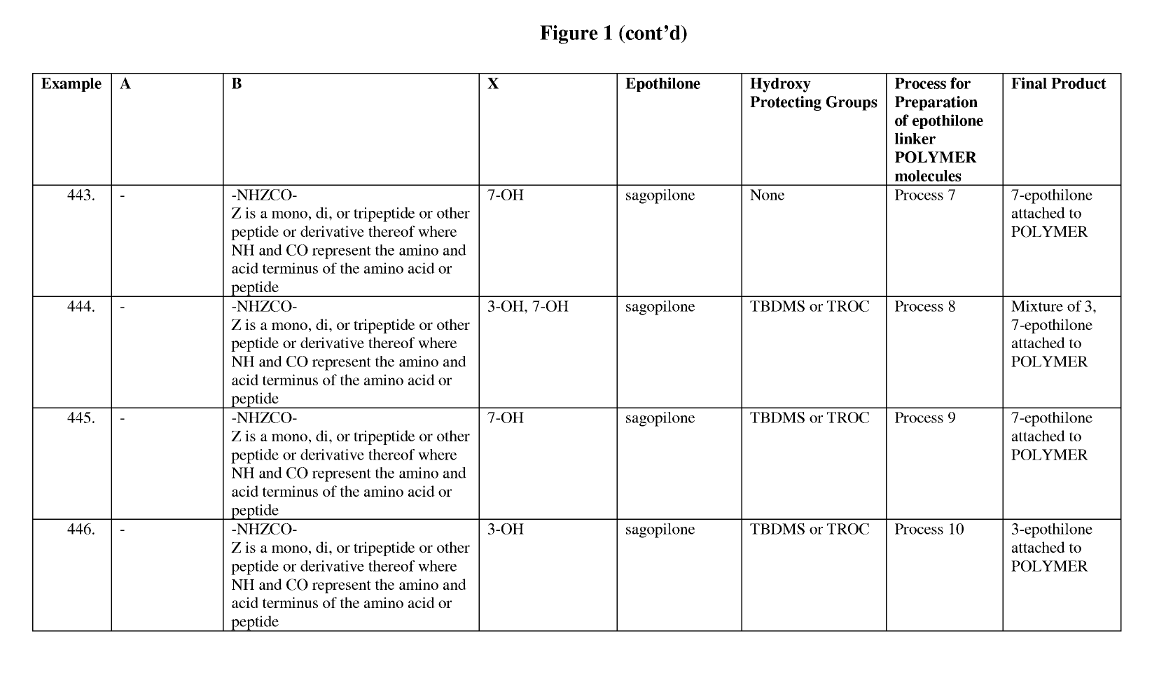

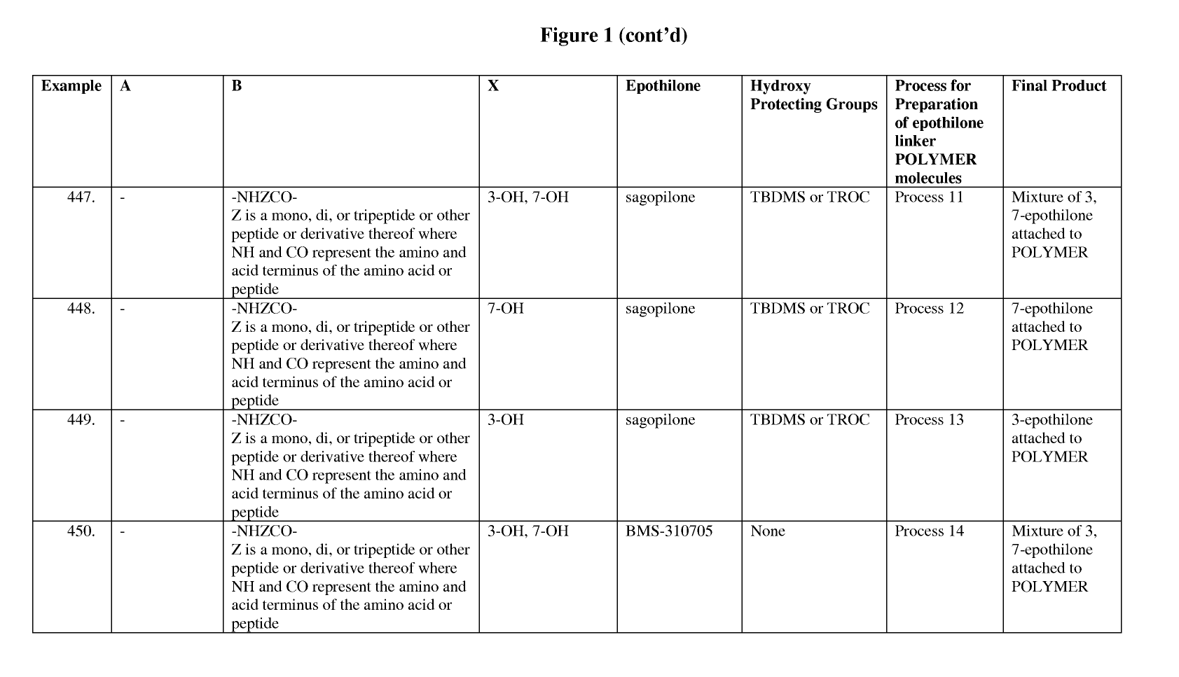

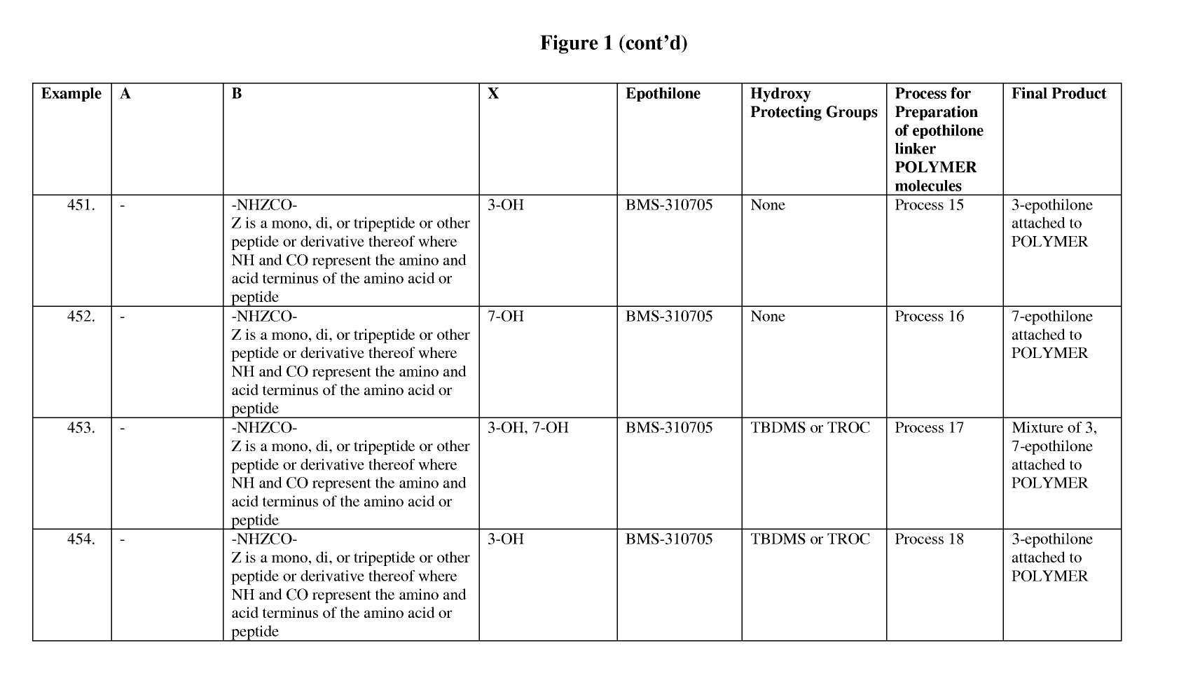

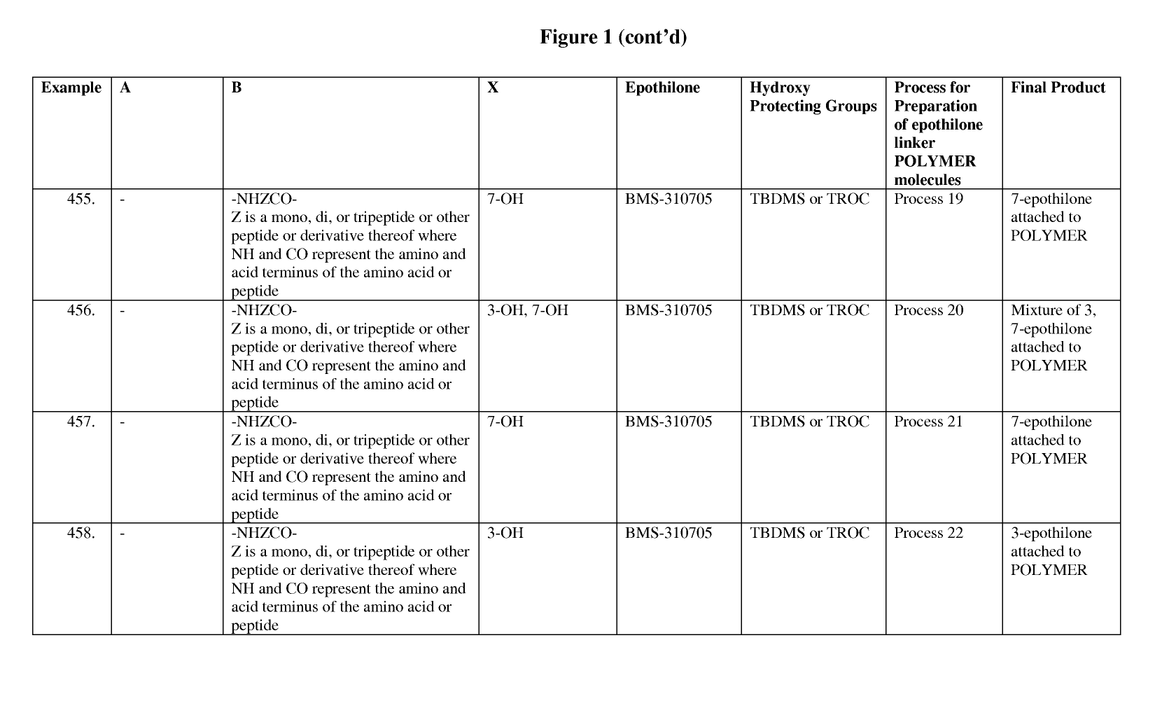

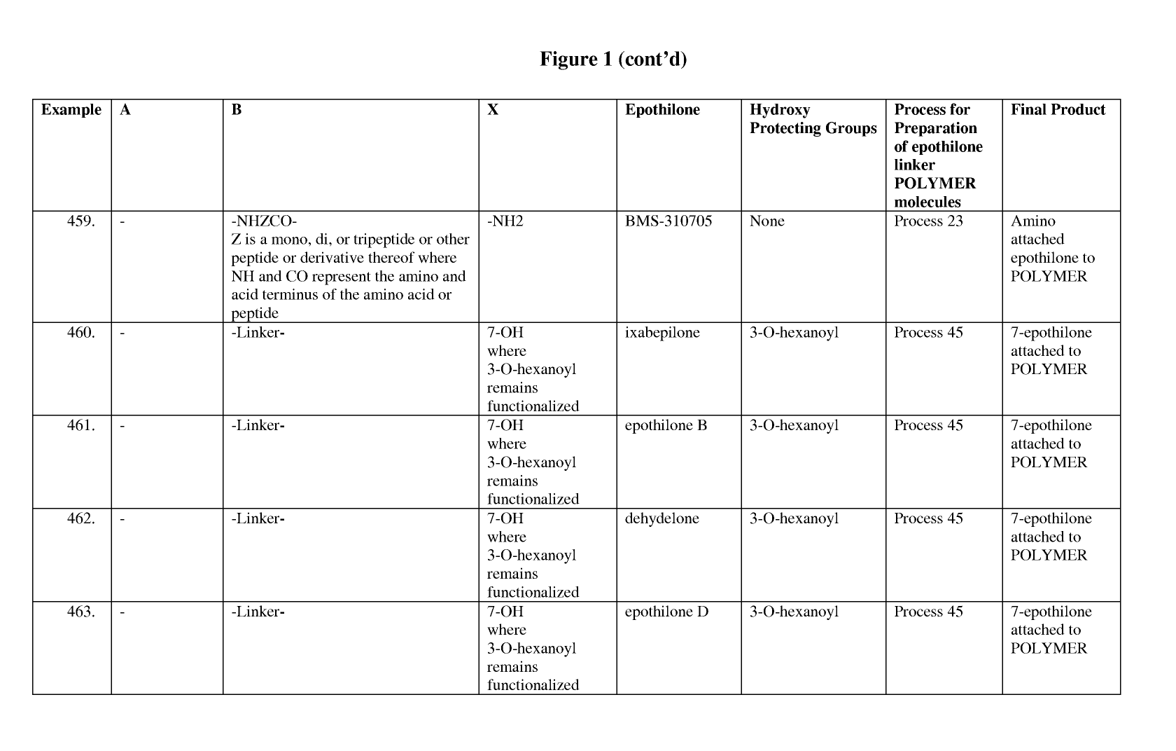

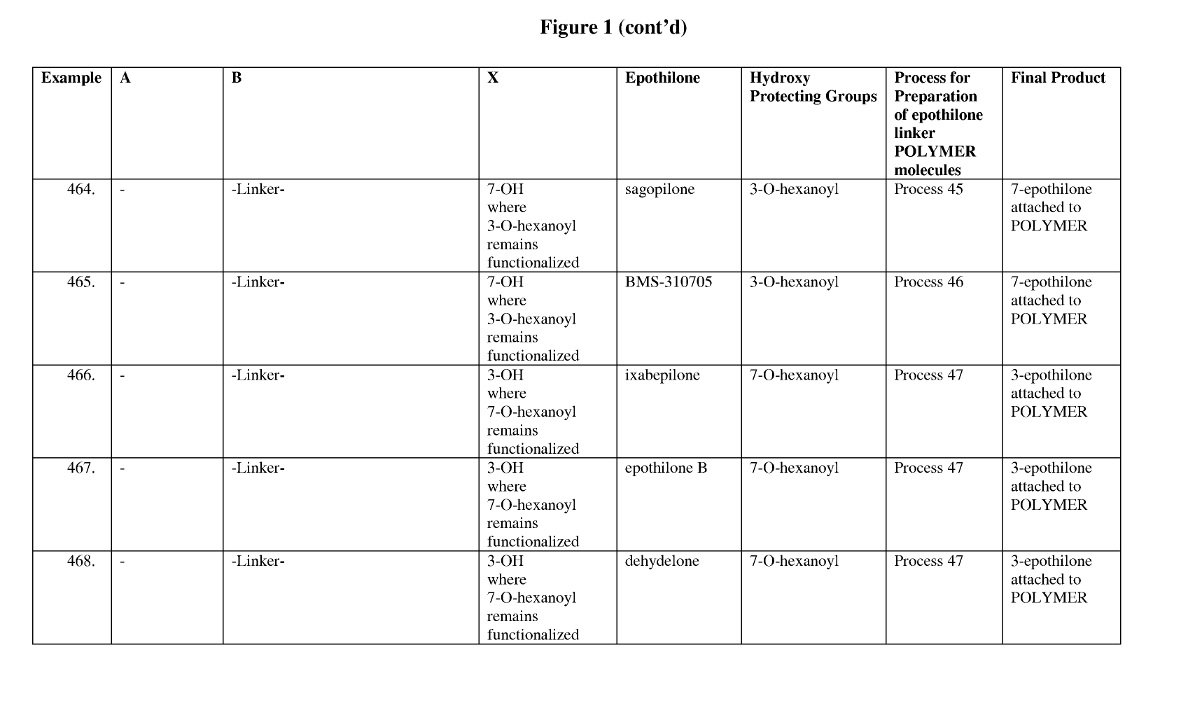

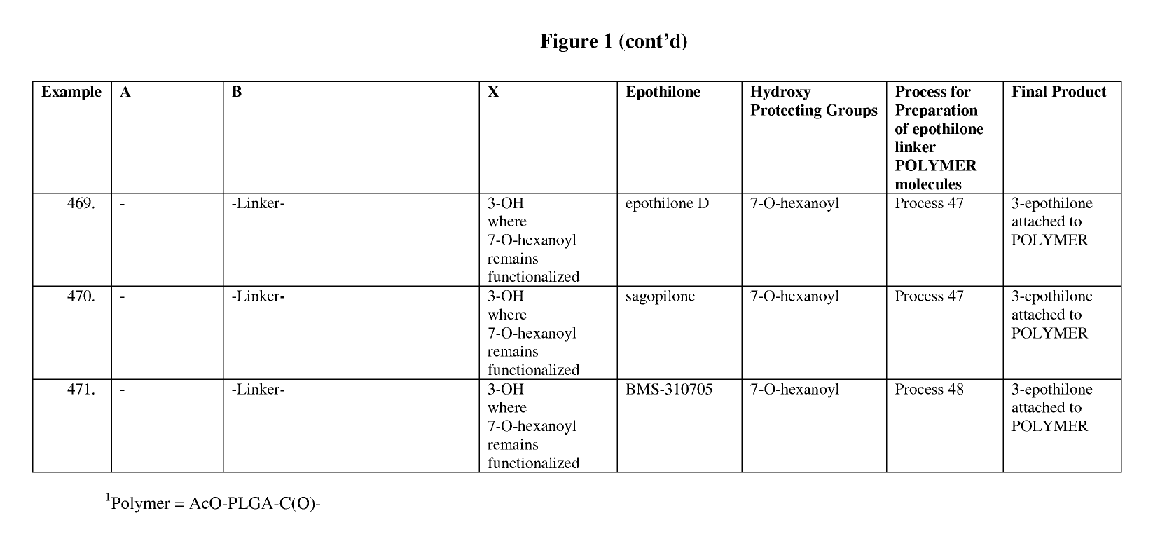

FIG. 1 is a table depicting examples of different polymer-agent conjugates. The polymer-agent conjugates in FIG. 1 are represented by the following formula:

### Polymer-ABX-Agent

“Polymer” in this formula represents the polymer portion of the polymer-agent conjugate. The polymer can be further modified on the end not conjugated with the agent. For example in instances where the polymer terminates with an —OH, the —OH can be capped, for example with an acyl group, as depicted in FIG. 1. In instances where the polymer terminates with a —COOH, the polymer may be capped, e.g., with an alkyl group to provide an ester.

A and B represent the connection between the polymer and the agent. Position A is either a bond between linker B and the carbonyl of the polymer (represented as a “-” in FIG. 1), a bond between the agent and the carbonyl of the polymer (represented as a “-” in FIG. 1) or depicts a portion of the linker that is attached via a bond to the carbonyl of the polymer. Position B is either not occupied (represented by “-” in FIG. 1) or represents the linker or the portion of the linker that is attached via a bond to the agent; and

X represents the heteroatom on the agent through which the linker or polymer is coupled to the agent.

As provided in FIG. 1, the column with the heading “drug” indicates which agent is included in the polymer-agent conjugate.

The three columns on the right of the table in FIG. 1 indicate respectively, what, if any, protecting groups are used to protect a hydroxy group on the agent, the process for producing the polymer-agent conjugate, and the final product of the process for producing the polymer-agent conjugate.

The processes referred to in FIG. 1 are given a numerical representation, e.g., Process 1, Process 2, Process 3 etc. as seen in the second column from the right. The steps for each these processes respectively are provided below.

Process 1: Couple the polymer directly to the epothilone to afford a mixture of 3- and 7-linked epothilone to polymer.

Process 2: Protect the epothilone, couple the protected epothilone via an unprotected hydroxyl group of the epothilone directly to the polymer, and deprotect to afford a mixture of 3- and 7-linked epothilone to polymer.

Process 3: Protect the epothilone, isolate the 3-protected epothilone, couple the 3-protected epothilone to the polymer and deprotect to afford a 7-linked epothilone to the polymer.

Process 4: Protect the epothilone, isolate the 7-protected epothilone, couple the 7-protected epothilone to the polymer and deprotect to afford 3-linked epothilone to polymer.

Process 5: Couple the protected linker of position B to the epothilone, deprotect the linker and couple to polymer via the carboxylic acid group of the polymer to afford a mixture of 3- and 7-linked epothilone to polymer.

Process 6: Couple the protected linker of position B to the epothilone, isolate 3-linked epothilone, and deprotect the linker and couple to polymer via the carboxylic acid group of the polymer to afford a 3-linked epothilone to polymer.

Process 7: Couple the protected linker of position B to the epothilone, isolate 7-linked epothilone, deprotect the linker and couple to polymer via the carboxylic acid group of the polymer to afford a 7-linked epothilone to polymer.

Process 8: Protect the epothilone, couple the protected linker of position B to an unprotected hydroxyl group of the epothilone, deprotect the linker and the epothilone hydroxyl protecting group, and couple to polymer via the carboxylic acid group of the polymer to afford a mixture of 3- and 7-linked epothilone to polymer.

Process 9: Protect the epothilone, isolate the 3-protected epothilone, couple the 3-protected epothilone to the protected linker of position B, deprotect linker and hydroxyl protecting group of the epothilone, and couple to polymer via the carboxylic acid group of the polymer to afford a 7-linked epothilone to polymer.

Process 10: Protect the epothilone, isolate the 7-protected epothilone, couple to the protected linker of position B, deprotect linker and hydroxyl protecting group of the epothilone, and couple to polymer via the carboxylic acid group of the polymer to afford 3-linked epothilone to polymer.

Process 11: Protect the epothilone, couple the protected linker of position B to an unprotected hydroxyl group of the epothilone, deprotect the linker protecting group, couple the linker to polymer via the carboxylic acid group of the polymer, and deprotect the hydroxyl protecting group to afford a mixture of 3- and 7-linked epothilone to polymer.

Process 12: Protect the epothilone, isolate the 3-protected epothilone, couple the 3-protected epothilone to the protected linker of position B, deprotect linker group of the epothilone, and couple to polymer via the carboxylic acid group of the polymer and then deprotect the hydroxyl group to afford a 7-linked epothilone to polymer.

Process 13: Protect the epothilone, isolate the 7-protected epothilone, couple to the protected linker of position B, deprotect linker group of the epothilone, and couple to polymer via the carboxylic acid group of the polymer to afford 3-linked epothilone to polymer and then deprotect the hydroxyl group to afford a 3-linked epothilone to polymer.

Process 14: Protect an amino group of the epothilone, couple the protected linker of position B to the epothilone, deprotect linker, couple to polymer via the carboxylic acid group of the polymer to afford a mixture of 3- and 7-linked epothilone to polymer, and deprotect the amino group of the epothilone and prepare salt to afford 7-linked epothilone.

Process 15: Protect an amino group of the epothilone, couple the protected linker of position B to the epothilone, isolate the 3-linked epothilone, deprotect the linker, couple to polymer via the carboxylic acid group of the polymer to afford 3-linked epothilone to polymer, and deprotect the amino group of the epothilone and prepare salt to afford 3-linked epothilone.

Process 16: Protect an amino group of the epothilone, couple the protected linker of position B to the epothilone, isolate the 7-linked epothilone, deprotect the linker, couple to polymer via the carboxylic acid group of the polymer to afford 7-linked epothilone to polymer, and deprotect the amino group of the epothilone and prepare salt to afford 7-linked epothilone.

Process 17: Protect an amino group and a hydroxyl group of the epothilone, couple the protected linker of position B to an unprotected hydroxyl group of the epothilone, deprotect the linker and the hydroxyl group of the epothilone, couple to polymer via the carboxylic acid group of the polymer to afford a mixture of 3- and 7-linked epothilone to polymer, and deprotect the amino group of the epothilone and prepare salt to afford a mixture of 3- and 7-linked eopthilone.

Process 18: Protect an amino group and a hydroxyl group of the epothilone, couple the protected linker of position B to an unprotected hydroxyl group of the epothilone, isolate the 3-linked epothilone, deprotect the linker and the hydroxyl group of the epothilone, couple to polymer via the carboxylic acid group of the polymer, deprotect amino group and prepare salt to afford a 3-linked epothilone to polymer.

Process 19: Protect an amino group and a hydroxyl group of the epothilone, couple the protected linker of position B to an unprotected hydroxyl group of the epothilone, isolate the 7-linked epothilone, deprotect the linker and the hydroxyl group of the epothilone, couple to polymer via the carboxylic acid group of the polymer, deprotect the amino group and prepare salt to afford a 7-linked epothilone to polymer.

Process 20: Protect epothilone amino group and hydroxyl group, couple the protected linker of position B to unprotected hydroxyl group, isolate the 3-linked epothilone, deprotect linker protecting group, couple to polymer via the carboxylic acid group of the polymer, deprotect hydroxyl and amino groups and prepare salt to afford a mixture of 3- and 7-linked epothilone to polymer

Process 21: Protect an amino group and a hydroxyl group of the epothilone, isolate 3-protected epothilone, couple the epothilone to the linker of position B, deprotect the linker protecting group, couple to polymer via the carboxylic acid group of the polymer to afford 7-linked epothilone to polymer, and deprotect the hydroxyl and amino groups of the epothilone and prepare salt to afford the 7-linked epothilone to polymer.

Process 22: Protect an amino group and a hydroxyl group of the epothilone, isolate 7-protected epothilone, couple the epothilone to the linker of position B, deprotect the linker of the epothilone, couple to polymer via the carboxylic acid group of the polymer to afford 3-linked epothilone to polymer, and deprotect the amino group and hydroxyl groups of the 3-epothilone and prepare salt to afford the 3-linked epothilone to polymer.

Process 23: Couple the protected linker of position B to an amino group of epothilone, deprotect the linker, and couple to polymer via the carboxylic acid group to afford NH-linked epothilone to polymer.

Process 24: Couple the activated linker of position B to the epothilone, and couple to polymer containing linker of position A via the linker of A to afford a mixture of 3- and 7-linked epothilone to polymer.

Process 25: Couple the activated linker of position B to the epothilone, isolate the 3-linked epothilone, and couple to the polymer containing linker of position A via the linker of A to afford the 3-linked epothilone to polymer.

Process 26: Couple the activated linker of position B, isolate the 7-linked epothilone, and couple to the polymer containing linker of position A via the linker of A to afford 7-linked epothilone to polymer.

Process 27: Protect one hydroxyl group of the epothilone, couple the activated linker of position B to an unprotected hydroxyl group of the epothilone, deprotect the hydroxyl group of the epothilone, and couple to the polymer containing linker of position A via the linker of A to afford a mixture of 3- and 7-linked epothilone to polymer.

Process 28—Protect one hydroxyl group of the epothilone, couple the activated linker of position B to an unprotected hydroxyl group of the epothilone, isolate the 3-linked epothiolone, deprotect the hydroxyl group of the epothilone, and couple to the polymer containing linker of position A via the linker of A to afford 3-linked epothilone to polymer.

Process 29—Protect one hydroxyl group of the epothilone, couple the activated linker of position B to an unprotected hydroxyl group of the epothilone, isolate the 7-linked epothiolone, deprotect the hydroxyl group of the epothilone, and couple to the polymer containing linker of position A via the linker of A to afford 3-linked epothilone to polymer.

Process 30: Protect one hydroxyl group of the epothilone, couple the activated linker of position B to an unprotected hydroxyl group of the epothilone, couple to the polymer containing linker of position A via the linker of A, and deprotect hydroxyl group of the epothilone to afford a mixture of 3- and 7-linked epothilone to polymer.

Process 31: Protect the epothilone, isolate the 3-protected epothilone, couple the epothilone to the activated linker of position B, couple to the polymer containing linker of position A to afford the 7-linked epothilone to polymer and deprotect hydroxyl group of the epothilone to afford 7-linked epothilone to polymer.

Process 32: Protect the epothilone, isolate the 7-protected epothilone, couple to the activated linker of position B, and couple to polymer containing linker of position A via the linker of A to afford the 3-linked epothilone to polymer and deprotect hydroxyl group of the epothilone to afford 3-linked epothilone to polymer.

Process 33: Couple the epothilone directly to polymer via the free amino group of the epothilone to the carboxylic acid group of the polymer to form NH-linked epothilone to polymer.

Process 34: Protect the amino group of the epothilone, couple the epothilone to polymer, deprotect the amino group, prepare salt to form 3,7-linked epothilone to polymer.

Process 35: Protect the amino group and one hydroxyl group of the epothilone, and couple the epothilone to polymer, deprotect the amino and the hydroxyl group, prepare salt to form 3,7-linked epothilone to polymer.

Process 36: Protect the amino group and one hydroxyl group of the epothilone, separate the 3-protected epothilone and couple the epothilone to polymer, deprotect the amino and the hydroxyl group, prepare salt to form 7-linked epothilone to polymer.

Process 37: Protect the amino group and one hydroxyl group of the epothilone, separate the 7-protected epothilone and couple the epothilone to polymer, deprotect the amino and the hydroxyl group, prepare salt to form 3-linked epothilone to polymer.

Process 38: Couple the activated linker of position B to the amino group of the epothilone and couple to the polymer containing linker of position A via the linker of A to afford an NH linked epothilone to polymer.

Process 39: Protect the amino group of the epothilone, couple the activated linker of position B to the hydroxyl groups of epothiolone, couple to the polymer containing the linker of position A via the linker of A, deprotect the amino group and prepare salt to afford 3,7-linked epothilone to polymer.

Process 40: Protect the amino group of the epothilone, couple the activated linker of position B to the hydroxyl groups of epothiolone, separate the 3-linked epothilone, couple to the polymer containing the linker of position A via the linker of A, deprotect the amino group and prepare salt to afford 3-linked epothilone to polymer.

Process 41: Protect the amino group of the epothilone, couple the activated linker of position B to the hydroxyl groups of epothiolone, separate the 7-linked epothilone, couple to the polymer containing the linker of position A via the linker of A, deprotect the amino group and prepare salt to afford 3-linked epothilone to polymer.

Process 42: Protect the amino group and one of the hydroxyl groups of the epothilone, couple the activated linker of position B to the hydroxyl group of epothiolone, couple to the polymer containing the linker of position A via the linker of A, deprotect the amino and the hydroxyl group and prepare salt to afford 3,7-linked epothilone to polymer.

Process 43: Protect the amino group and one of the hydroxyl groups of the epothilone, separate the 3-protected epothilone, couple the activated linker of position B to the hydroxyl group of epothiolone, couple to the polymer containing the linker of position A via the linker of A, deprotect the amino and the hydroxyl group and prepare salt to afford 7-linked epothilone to polymer.

Process 44: Protect the amino group and one of the hydroxyl groups of the epothilone, separate the 7-protected epothilone, couple the activated linker of position B to the hydroxyl group of epothiolone, couple to the polymer containing the linker of position A via the linker of A, deprotect the amino and the hydroxyl group and prepare salt to afford 3-linked epothilone to polymer.

Process 45: Protect the epothilone, isolate the 3-protected epothilone, couple the epothilone to the linker of position B, deprotect the linker, and couple to polymer via the carboxylic acid group of polymer to afford the 7-linked epothilone to polymer.

Process 46: Protect the amino and hydroxyl groups of the epothilone, isolate 3-protected epothilone, couple to the protected linker of position B, deprotect the linker, and couple to polymer via the carboxylic acid group of polymer to afford 7-linked epothilone to polymer.

Process 47: Protect the epothilone, isolate the 7-protected epothilone, couple to the protected linker of position B, deprotect linker, and couple to polymer via the carboxylic acid group of polymer to afford the 3-linked epothilone to polymer.

Process 48: Protect the amino and hydroxyl groups of the epothilone, isolate 7-protected epothilone, couple to the protected linker of position B, deprotect the linker, and couple to polymer via the carboxylic acid group of the polymer to afford 3-linked epothilone to polymer.

### Compositions of Polymer-Agent Conjugates

Compositions of polymer-agent conjugates described above may include mixtures of products. For example, the conjugation of an agent to a polymer may proceed in less than 100% yield, and the composition comprising the polymer-agent conjugate may thus also include unconjugated polymer.

Compositions of polymer-agent conjugates may also include polymer-agent conjugates that have the same polymer and the same agent, and differ in the nature of the linkage between the agent and the polymer. For example, in some embodiments, when the agent is an epothilone, the composition may include polymers attached to the agent via different hydroxyl groups present on the agent. In the case of an epothilone, the composition may include polymers attached to the epothilone via the hydroxyl group at the 3 position and/or polymers attached to the epothilone via the hydroxyl group at the 7 position. The polymer-agent conjugates may be present in the composition in varying amounts. For example, when an agent having a plurality of available attachment points (e.g., an epothilone) is reacted with a polymer, the resulting composition may include more of a product conjugated via a one hydroxyl group, and less of a product attached via another hydroxyl group.

Additionally, compositions of polymer-agent conjugates may include agents that are attached to more than one polymer chain. For example, in the case of an epothilone, the composition may include an epothilone attached to one polymer chain via the hydroxyl group at the 3 position and a second polymer chain via the hydroxyl group at the 7 position.

### Particles

In general, a particle described herein includes a hydrophobic polymer, a polymer containing a hydrophilic portion and a hydrophobic portion, and one or more agents (e.g., epothilones). In some embodiments, an agent may be attached to a polymer (e.g., a hydrophobic polymer or a polymer containing a hydrophilic and a hydrophobic portion), and in some embodiments, an additional agent may be embedded in the particle. In some embodiments, an agent may not be attached to a polymer and may be embedded in the particle. The additional agent may be the same as the agent attached to a polymer, or may be a different agent. A particle described herein may also include a compound having at least one acidic moiety, such as a carboxylic acid group. The compound may be a small molecule or a polymer having at least one acidic moiety. In some embodiments, the compound is a polymer such as PLGA. A particle described herein may also include one or more excipients, such as surfactants, stabilizers or lyoprotectants. Exemplary stabilizers or lyoprotectants include carbohydrates (e.g., a carbohydrate described herein, such as, e.g., sucrose, cyclodextrin or a derivative of cyclodextrin (e.g. 2-hydroxypropyl-β-cyclodextrin)), salt, PEG, PVP, crown either or polyol (e.g., trehalose, mannitol, sorbitol or lactose).

In some embodiments, the particle is a nanoparticle. In some embodiments, the nanoparticle has a diameter of less than or equal to about 220 nm (e.g., less than or equal to about 215 nm, 210 nm, 205 nm, 200 nm, 195 nm, 190 nm, 185 nm, 180 nm, 175 nm, 170 nm, 165 nm, 160 nm, 155 nm, 150 nm, 145 nm, 140 nm, 135 nm, 130 nm, 125 nm, 120 nm, 115 nm, 110 nm, 105 nm, 100 nm, 95 nm, 90 nm, 85 nm, 80 nm, 75 nm, 70 nm, 65 nm, 60 nm, 55 nm or 50 nm).

A composition of a plurality of particles described herein may have an average diameter of about 50 nm to about 500 nm (e.g., from about 50 nm to about 200 nm). A composition of a plurality of particles particle may have a median particle size (Dv50) is from about 50 nm to about 220 nm (e.g., from about 75 nm to about 200 nm). A composition of a plurality of particles particle may have a Dv90 (particle size below which 90% of the volume of particles exists) of about 50 nm to about 500 nm (e.g., about 75 nm to about 220 nm).

A particle described herein may have a surface zeta potential ranging from about −80 mV to about 50 mV, when measured in water. Zeta potential is a measurement of surface potential of a particle. In some embodiments, a particle may have a surface zeta potential, when measured in water, ranging between about −50 mV to about 30 mV, about −20 mV to about 20 mV, or about −10 mV to about 10 mV. In some embodiments, the zeta potential of the particle surface, when measured in water, is neutral or slightly negative. In some embodiments, the zeta potential of the particle surface, when measured in water, is less than 0, e.g., 0 to −20 mV.

A particle described herein may include a small amount of a residual solvent, e.g., a solvent used in preparing the particles such as acetone, tert-butylmethyl ether, heptane, dichloromethane, dimethylformamide, ethyl acetate, acetonitrile, tetrahydrofuran, ethanol, methanol, isopropyl alcohol, methyl ethyl ketone, butyl acetate, or propyl acetate. In some embodiments, the particle may include less than 5000 ppm of a solvent (e.g., less than 4500 ppm, less than 4000 ppm, less than 3500 ppm, less than 3000 ppm, less than 2500 ppm, less than 2000 ppm, less than 1500 ppm, less than 1000 ppm, less than 500 ppm, less than 250 ppm, less than 100 ppm, less than 50 ppm, less than 25 ppm, less than 10 ppm, less than 5 ppm, less than 2 ppm, or less than 1 ppm).

In some embodiments, the particle is substantially free of a class II or class III solvent as defined by the United States Department of Health and Human Services Food and Drug Administration “Q3c-Tables and List.” In some embodiments, the particle comprises less than 5000 ppm of acetone. In some embodiments, the particle comprises less than 5000 ppm of tert-butylmethyl ether. In some embodiments, the particle comprises less than 5000 ppm of heptane. In some embodiments, the particle comprises less than 600 ppm of dichloromethane. In some embodiments, the particle comprises less than 880 ppm of dimethylformamide. In some embodiments, the particle comprises less than 5000 ppm of ethyl acetate. In some embodiments, the particle comprises less than 410 ppm of acetonitrile. In some embodiments, the particle comprises less than 720 ppm of tetrahydrofuran. In some embodiments, the particle comprises less than 5000 ppm of ethanol. In some embodiments, the particle comprises less than 3000 ppm of methanol. In some embodiments, the particle comprises less than 5000 ppm of isopropyl alcohol. In some embodiments, the particle comprises less than 5000 ppm of methyl ethyl ketone. In some embodiments, the particle comprises less than 5000 ppm of butyl acetate. In some embodiments, the particle comprises less than 5000 ppm of propyl acetate.

A particle described herein may include varying amounts of a hydrophobic polymer, e.g., from about 20% to about 90% (e.g., from about 20% to about 80%, from about 25% to about 75%, or from about 30% to about 70%). A particle described herein may include varying amounts of a polymer containing a hydrophilic portion and a hydrophobic portion, e.g., up to about 50% by weight (e.g., from about 4 to any of about 50%, about 5%, about 8%, about 10%, about 15%, about 20%, about 23%, about 25%, about 30%, about 35%, about 40%, about 45% or about 50% by weight). For example, the percent by weight of the second polymer within the particle is from about 3% to 30%, from about 5% to 25% or from about 8% to 23%.

A particle described herein may be substantially free of a targeting agent (e.g., of a targeting agent covalently linked to the particle, e.g., to the first or second polymer or agent), e.g., a targeting agent able to bind to or otherwise associate with a target biological entity, e.g., a membrane component, a cell surface receptor, prostate specific membrane antigen, or the like. A particle described herein may be substantially free of a targeting agent that causes the particle to become localized to a tumor, a disease site, a tissue, an organ, a type of cell, e.g., a cancer cell, within the body of a subject to whom a therapeutically effective amount of the particle is administered. A particle described herein may be substantially free of a targeting agent selected from nucleic acid aptamers, growth factors, hormones, cytokines, interleukins, antibodies, integrins, fibronectin receptors, p-glycoprotein receptors, peptides and cell binding sequences. In some embodiments, no polymer within the particle is conjugated to a targeting moiety. In an embodiment substantially free of a targeting agent means substantially free of any moiety other than the first polymer, the second polymer, a third polymer (if present), a surfactant (if present), and the agent, e.g., an epothilone or anti-cancer agent, that targets the particle. Thus, in such embodiments, any contribution to localization by the first polymer, the second polymer, a third polymer (if present), a surfactant (if present), and the agent is not considered to be “targeting.” A particle described herein may be free of moieties added for the purpose of selectively targeting the particle to a site in a subject, e.g., by the use of a moiety on the particle having a high and specific affinity for a target in the subject.

In some embodiments the second polymer is other than a lipid, e.g., other than a phospholipid. A particle described herein may be substantially free of an amphiphilic layer that reduces water penetration into the nanoparticle. A particle described herein may comprise less than 5 or 10% (e.g., as determined as w/w, v/v) of a lipid, e.g., a phospholipid. A particle described herein may be substantially free of a lipid layer, e.g., a phospholipid layer, e.g., that reduces water penetration into the nanoparticle. A particle described herein may be substantially free of lipid, e.g., is substantially free of phospholipid.

A particle described herein may be substantially free of a radiopharmaceutical agent, e.g., a radiotherapeutic agent, radiodiagnostic agent, prophylactic agent, or other radioisotope. A particle described herein may be substantially free of an immunomodulatory agent, e.g., an immunostimulatory agent or immunosuppressive agent. A particle described herein may be substantially free of a vaccine or immunogen, e.g., a peptide, sugar, lipid-based immunogen, B cell antigen or T cell antigen.

A particle described herein may be substantially free of a water-soluble hydrophobic polymer such as PLGA, e.g., PLGA having a molecular weight of less than about 1 kDa.

In a particle described herein, the ratio of the first polymer to the second polymer is such that the particle comprises at least 5%, 8%, 10%, 12%, 15%, 18%, 20%, 23%, 25%, or 30% by weight of a polymer having a hydrophobic portion and a hydrophilic portion.

Methods of Making Particles and Compositions

A particle described herein may be prepared using any method known in the art for preparing particles, e.g., nanoparticles. Exemplary methods include spray drying, emulsion (e.g., emulsion-solvent evaporation or double emulsion), precipitation (e.g., nanoprecipitation) and phase inversion.

In one embodiment, a particle described herein can be prepared by precipitation (e.g., nanoprecipitation). This method involves dissolving the components of the particle (i.e., one or more polymers, an optional additional component or components, and an agent), individually or combined, in one or more solvents to form one or more solutions. For example, a first solution containing one or more of the components may be poured into a second solution containing one or more of the components (at a suitable rate or speed). The solutions may be combined, for example, using a syringe pump, a MicroMixer, or any device that allows for vigorous, controlled mixing. In some cases, nanoparticles can be formed as the first solution contacts the second solution, e.g., precipitation of the polymer upon contact causes the polymer to form nanoparticles. The control of such particle formation can be readily optimized.

In one set of embodiments, the particles are formed by providing one or more solutions containing one or more polymers and additional components, and contacting the solutions with certain solvents to produce the particle. In a non-limiting example, a hydrophobic polymer (e.g., PLGA), is conjugated to an agent to form a conjugate. This polymer-agent conjugate, a polymer containing a hydrophilic portion and a hydrophobic portion (e.g., PEG-PLGA), and optionally a third polymer (e.g., a biodegradable polymer, e.g., PLGA) are dissolved in a partially water miscible organic solvent (e.g., acetone). This solution is added to an aqueous solution containing a surfactant, forming the desired particles. These two solutions may be individually sterile filtered prior to mixing/precipitation.

The formed nanoparticles can be exposed to further processing techniques to remove the solvents or purify the nanoparticles (e.g., dialysis). For purposes of the aforementioned process, water miscible solvents include acetone, ethanol, methanol, and isopropyl alcohol; and partially water miscible organic solvents include acetonitrile, tetrahydrofuran, ethyl acetate, isopropyl alcohol, isopropyl acetate or dimethylformamide.

Another method that can be used to generate a particle described herein is a process termed “flash nanoprecipitation” as described by Johnson, B. K., et al, AlChE Journal (2003) 49:2264-2282 and U.S. 2004/0091546, each of which is incorporated herein by reference in its entirety. This process is capable of producing controlled size, polymer-stabilized and protected nanoparticles of hydrophobic organics at high loadings and yields. The flash nanoprecipitation technique is based on amphiphilic diblock copolymer arrested nucleation and growth of hydrophobic organics. Amphiphilic diblock copolymers dissolved in a suitable solvent can form micelles when the solvent quality for one block is decreased. In order to achieve such a solvent quality change, a tangential flow mixing cell (vortex mixer) is used. The vortex mixer consists of a confined volume chamber where one jet stream containing the diblock copolymer and active agent dissolved in a water-miscible solvent is mixed at high velocity with another jet stream containing water, an anti-solvent for the active agent and the hydrophobic block of the copolymer. The fast mixing and high energy dissipation involved in this process provide timescales that are shorter than the timescale for nucleation and growth of particles, which leads to the formation of nanoparticles with active agent loading contents and size distributions not provided by other technologies. When forming the nanoparticles via flash nanoprecipitation, mixing occurs fast enough to allow high supersaturation levels of all components to be reached prior to the onset of aggregation. Therefore, the active agent(s) and polymers precipitate simultaneously, and overcome the limitations of low active agent incorporations and aggregation found with the widely used techniques based on slow solvent exchange (e.g., dialysis). The flash nanoprecipitation process is insensitive to the chemical specificity of the components, making it a universal nanoparticle formation technique.

A particle described herein may also be prepared using a mixer technology, such as a static mixer or a micro-mixer (e.g., a split-recombine micro-mixer, a slit-interdigital micro-mixer, a star laminator interdigital micro-mixer, a superfocus interdigital micro-mixer, a liquid-liquid micro-mixer, or an impinging jet micro-mixer).

A split-recombine micromixer uses a mixing principle involving dividing the streams, folding/guiding over each other and recombining them per each mixing step, consisting of 8 to 12 such steps. Mixing finally occurs via diffusion within milliseconds, exclusive of residence time for the multi-step flow passage. Additionally, at higher-flow rates, turbulences add to this mixing effect, improving the total mixing quality further.

A slit interdigital micromixer combines the regular flow pattern created by multi-lamination with geometric focusing, which speeds up liquid mixing. Due to this double-step mixing, a slit mixer is amenable to a wide variety of processes.

A particle described herein may also be prepared using Microfluidics Reaction Technology (MRT). At the core of MRT is a continuous, impinging jet microreactor scalable to at least 50 lit/min. In the reactor, high-velocity liquid reactants are forced to interact inside a microliter scale volume. The reactants mix at the nanometer level as they are exposed to high shear stresses and turbulence. MRT provides precise control of the feed rate and the mixing location of the reactants. This ensures control of the nucleation and growth processes, resulting in uniform crystal growth and stabilization rates.

A particle described herein may also be prepared by emulsion. An exemplary emulsification method is disclosed in U.S. Pat. No. 5,407,609, which is incorporated herein by reference. This method involves dissolving or otherwise dispersing agents, liquids or solids, in a solvent containing dissolved wall-forming materials, dispersing the agent/polymer-solvent mixture into a processing medium to form an emulsion and transferring all of the emulsion immediately to a large volume of processing medium or other suitable extraction medium, to immediately extract the solvent from the microdroplets in the emulsion to form a microencapsulated product, such as microcapsules or microspheres. The most common method used for preparing polymer delivery vehicle formulations is the solvent emulsification-evaporation method. This method involves dissolving the polymer and drug in an organic solvent that is completely immiscible with water (for example, dichloromethane). The organic mixture is added to water containing a stabilizer, most often poly(vinyl alcohol) (PVA) and then typically sonicated.

After the particles are prepared, they may be fractionated by filtering, sieving, extrusion, or ultracentrifugation to recover particles within a specific size range. One sizing method involves extruding an aqueous suspension of the particles through a series of polycarbonate membranes having a selected uniform pore size; the pore size of the membrane will correspond roughly with the largest size of particles produced by extrusion through that membrane. See, e.g., U.S. Pat. No. 4,737,323, incorporated herein by reference. Another method is serial ultracentrifugation at defined speeds (e.g., 8,000, 10,000, 12,000, 15,000, 20,000, 22,000, and 25,000 rpm) to isolate fractions of defined sizes. Another method is tangential flow filtration, wherein a solution containing the particles is pumped tangentially along the surface of a membrane. An applied pressure serves to force a portion of the fluid through the membrane to the filtrate side. Particles that are too large to pass through the membrane pores are retained on the upstream side. The retained components do not build up at the surface of the membrane as in normal flow filtration, but instead are swept along by the tangential flow. Tangential flow filtration may thus be used to remove excess surfactant present in the aqueous solution or to concentrate the solution via diafiltration.

After purification of the particles, they may be sterile filtered (e.g., using a 0.22 micron filter) while in solution.

In certain embodiments, the particles are prepared to be substantially homogeneous in size within a selected size range. The particles are preferably in the range from 30 nm to 300 nm in their greatest diameter, (e.g., from about 30 nm to about 250 nm). The particles may be analyzed by techniques known in the art such as dynamic light scattering and/or electron microscopy, (e.g., transmission electron microscopy or scanning electron microscopy) to determine the size of the particles. The particles may also be tested for agent loading and/or the presence or absence of impurities.

Lyophilization

A particle described herein may be prepared for dry storage via lyophilization, commonly known as freeze-drying. Lyophilization is a process which extracts water from a solution to form a granular solid or powder. The process is carried out by freezing the solution and subsequently extracting any water or moisture by sublimation under vacuum. Advantages of lyophilization include maintenance of substance quality and minimization of therapeutic compound degradation. Lyophilization may be particularly useful for developing pharmaceutical drug products that are reconstituted and administered to a patient by injection, for example parenteral drug products. Alternatively, lyophilization is useful for developing oral drug products, especially fast melts or flash dissolve formulations.

Lyophilization may take place in the presence of a lyoprotectant, e.g., a lyoprotectant described herein. In some embodiments, the lyoprotectant is a carbohydrate (e.g., a carbohydrate described herein, such as, e.g., sucrose, cyclodextrin or a derivative of cyclodextrin (e.g. 2-hydroxypropyl-β-cyclodextrin)), salt, PEG, PVP or crown ether.

Methods of Storing

A polymer-agent conjugate, particle or composition described herein may be stored in a container for at least about 1 hour (e.g., at least about 2 hours, 4 hours, 8 hours, 12 hours, 24 hours, 2 days, 1 week, 1 month, 2 months, 3 months, 4 months, 5 months, 6 months, 1 year, 2 years or 3 years). Accordingly, described herein are containers including a polymer-agent conjugate, particle or composition described herein.

A polymer-agent conjugate, particle or composition may be stored under a variety of conditions, including ambient conditions (e.g., at room temperature, ambient humidity, and atmospheric pressure). A polymer-agent conjugate, particle or composition may also be stored at low temperature, e.g., at a temperature less than or equal to about 5° C. (e.g., less than or equal to about 4° C. or less than or equal to about 0° C.). A polymer-agent conjugate, particle or composition may also be frozen and stored at a temperature of less than about 0° C. (e.g., between −80° C. and −20° C.). A polymer-agent conjugate, particle or composition may also be stored under an inert atmosphere, e.g., an atmosphere containing an inert gas such as nitrogen or argon. Such an atmosphere may be substantially free of atmospheric oxygen and/or other reactive gases, and/or substantially free of moisture.

A polymer-agent conjugate, particle or composition described herein may be stored in a variety of containers, including a light-blocking container such as an amber vial. A container may be a vial, e.g., a sealed vial having a rubber or silicone enclosure (e.g., an enclosure made of polybutadiene or polyisoprene). A container may be substantially free of atmospheric oxygen and/or other reactive gases, and/or substantially free of moisture.

Methods of Evaluating Particles

A particle described herein may be subjected to a number of analytical methods. For example, a particle described herein may be subjected to a measurement to determine whether an impurity or residual solvent is present (e.g., via gas chromatography (GC)), to determine relative amounts of one or more components (e.g., via high performance liquid chromatography (HPLC)), to measure particle size (e.g., via dynamic light scattering and/or scanning electron microscopy), or determine the presence or absence of surface components.

In some embodiments, a particle described herein may be evaluated using dynamic light scattering. Particles may be illuminated with a laser, and the intensity of the scattered light fluctuates at a rate that is dependent upon the size of the particles as smaller particles are “kicked” further by the solvent molecules and move more rapidly. Analysis of these intensity fluctuations yields the velocity of the Brownian motion and hence the particle size using the Stokes-Einstein relationship. The diameter that is measured in Dynamic Light Scattering is called the hydrodynamic diameter and refers to how a particle diffuses within a fluid. The diameter obtained by this technique is that of a sphere that has the same translational diffusion coefficient as the particle being measured.

In some embodiments, a particle described herein may be evaluated using cryo scanning electron microscopy (Cryo-SEM). SEM is a type of electron microscopy in which the sample surface is imaged by scanning it with a high-energy beam of electrons in a raster scan pattern. The electrons interact with the atoms that make up the sample producing signals that contain information about the sample's surface topography, composition and other properties such as electrical conductivity. For Cryo-SEM, the SEM is equipped with a cold stage for cryo-microscopy. Cryofixation may be used and low-temperature scanning electron microscopy performed on the cryogenically fixed specimens. Cryo-fixed specimens may be cryo-fractured under vacuum in a special apparatus to reveal internal structure, sputter coated and transferred onto the SEM cryo-stage while still frozen.

In some embodiments, a particle described herein may be evaluated using transmission electron microscopy (TEM). In this technique, a beam of electrons is transmitted through an ultra thin specimen, interacting with the specimen as it passes through. An image is formed from the interaction of the electrons transmitted through the specimen; the image is magnified and focused onto an imaging device, such as a fluorescent screen, on a layer of photographic film, or to be detected by a sensor such as a charge-coupled device (CCD) camera.

### Pharmaceutical Compositions

In another aspect, the present invention provides a composition, e.g., a pharmaceutical composition, comprising a plurality of particles described herein and a pharmaceutically acceptable carrier or adjuvant.

In some embodiments, a pharmaceutical composition may include a pharmaceutically acceptable salt of a compound described herein, e.g., a polymer-agent conjugate. Pharmaceutically acceptable salts of the compounds described herein include those derived from pharmaceutically acceptable inorganic and organic acids and bases. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-naphthalenesulfonate, nicotinate, nitrate, palmoate, phosphate, picrate, pivalate, propionate, salicylate, succinate, sulfate, tartrate, tosylate and undecanoate. Salts derived from appropriate bases include alkali metal (e.g., sodium), alkaline earth metal (e.g., magnesium), ammonium and N-(alkyl)₄⁺ salts. This invention also envisions the quaternization of any basic nitrogen-containing groups of the compounds described herein. Water or oil-soluble or dispersible products may be obtained by such quaternization.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gailate, alpha-tocopherol, and the like; and (3) metal chelating agents, such as citric acid, ethylenediamine tetraacetic acid (EDTA), sorbitol, tartaric acid, phosphoric acid, and the like.

A composition may include a liquid used for suspending a polymer-agent conjugate, particle or composition, which may be any liquid solution compatible with the polymer-agent conjugate, particle or composition, which is also suitable to be used in pharmaceutical compositions, such as a pharmaceutically acceptable nontoxic liquid. Suitable suspending liquids including but are not limited to suspending liquids selected from the group consisting of water, aqueous sucrose syrups, corn syrups, sorbitol, polyethylene glycol, propylene glycol, D5W and mixtures thereof.

A composition described herein may also include another component, such as an antioxidant, antibacterial, buffer, bulking agent, chelating agent, an inert gas, a tonicity agent and/or a viscosity agent.

In one embodiment, the polymer-agent conjugate, particle or composition is provided in lyophilized form and is reconstituted prior to administration to a subject. The lyophilized polymer-agent conjugate, particle or composition can be reconstituted by a diluent solution, such as a salt or saline solution, e.g., a sodium chloride solution having a pH between 6 and 9, lactated Ringer's injection solution, or a commercially available diluent, such as PLASMA-LYTE A Injection pH 7.4® (Baxter, Deerfield, Ill.).

In one embodiment, a lyophilized formulation includes a lyoprotectant or stabilizer to maintain physical and chemical stability by protecting the particle and active from damage from crystal formation and the fusion process during freeze-drying. The lyoprotectant or stabilizer can be one or more of polyethylene glycol (PEG), a PEG lipid conjugate (e.g., PEG-ceramide or D-alpha-tocopheryl polyethylene glycol 1000 succinate), poly(vinyl alcohol) (PVA), poly(vinylpyrrolidone) (PVP), polyoxyethylene esters, poloxamers, polysorbates, polyoxyethylene esters, lecithins, saccharides, oligosaccharides, polysaccharides, carbohydrates, cyclodextrins (e.g. 2-hydroxypropyl-β-cyclodextrin) and polyols (e.g., trehalose, mannitol, sorbitol, lactose, sucrose, glucose and dextran), salts and crown ethers.

In some embodiments, the lyophilized polymer-agent conjugate, particle or composition is reconstituted with water, 5% Dextrose Injection, Lactated Ringer's and Dextrose Injection, or a mixture of equal parts by volume of Dehydrated Alcohol, USP and a nonionic surfactant, such as a polyoxyethylated castor oil surfactant available from GAF Corporation, Mount Olive, N.J., under the trademark, Cremophor EL. The lyophilized product and vehicle for reconstitution can be packaged separately in appropriately light-protected vials. To minimize the amount of surfactant in the reconstituted solution, only a sufficient amount of the vehicle may be provided to form a solution of the polymer-agent conjugate, particle or composition. Once dissolution of the drug is achieved, the resulting solution is further diluted prior to injection with a suitable parenteral diluent. Such diluents are well known to those of ordinary skill in the art. These diluents are generally available in clinical facilities. It is, however, within the scope of the present invention to package the subject polymer-agent conjugate, particle or composition with a third vial containing sufficient parenteral diluent to prepare the final concentration for administration. A typical diluent is Lactated Ringer's Injection.

The final dilution of the reconstituted polymer-agent conjugate, particle or composition may be carried out with other preparations having similar utility, for example, 5% Dextrose Injection, Lactated Ringer's and Dextrose Injection, Sterile Water for Injection, and the like. However, because of its narrow pH range, pH 6.0 to 7.5, Lactated Ringer's Injection is most typical. Per 100 mL, Lactated Ringer's Injection contains Sodium Chloride USP 0.6 g, Sodium Lactate 0.31 g, Potassium chloride USP 0.03 g and Calcium Chloride2H2O USP 0.02 g. The osmolarity is 275 mOsmol/L, which is very close to isotonicity.

The compositions may conveniently be presented in unit dosage form and may be prepared by any methods well known in the art of pharmacy. The amount of active agent which can be combined with a pharmaceutically acceptable carrier to produce a single dosage form will vary depending upon the host being treated, the particular mode of administration. The amount of active agent which can be combined with a pharmaceutically acceptable carrier to produce a single dosage form will generally be that amount of the compound which produces a therapeutic effect.

### Routes of Administration

The pharmaceutical compositions described herein may be administered orally, parenterally (e.g., via intravenous, subcutaneous, intracutaneous, intramuscular, intraarticular, intraarterial, intrasynovial, intrasternal, intrathecal, intralesional or intracranial injection), topically, mucosally (e.g., rectally or vaginally), nasally, buccally, ophthalmically, via inhalation spray (e.g., delivered via nebulization, propellant or a dry powder device) or via an implanted reservoir.

Pharmaceutical compositions suitable for parenteral administration comprise one or more polymer-agent conjugate(s), particle(s) or composition(s) in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the agent from subcutaneous or intramuscular injection. This may be accomplished by the use of a liquid suspension of crystalline or amorphous material having poor water solubility. The rate of absorption of the polymer-agent conjugate, particle or composition then depends upon its rate of dissolution which, in turn, may depend upon crystal size and crystalline form. Alternatively, delayed absorption of a parenterally administered drug form is accomplished by dissolving or suspending the polymer-agent conjugate, particle or composition in an oil vehicle.

Pharmaceutical compositions suitable for oral administration may be in the form of capsules, cachets, pills, tablets, gums, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouthwashes and the like, each containing a predetermined amount of an agent as an active ingredient. A compound may also be administered as a bolus, electuary or paste.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered peptide or peptidomimetic moistened with an inert liquid diluent.

Tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups and elixirs. In addition to the polymer-agent conjugate, particle or composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the polymer-agent conjugate, particle or composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions suitable for topical administration are useful when the desired treatment involves areas or organs readily accessible by topical application. For application topically to the skin, the pharmaceutical composition should be formulated with a suitable ointment containing the active components suspended or dissolved in a carrier. Carriers for topical administration of the a particle described herein include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active particle suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions described herein may also be topically applied to the lower intestinal tract by rectal suppository formulation or in a suitable enema formulation. Topically-transdermal patches are also included herein.

The pharmaceutical compositions described herein may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

The pharmaceutical compositions described herein may also be administered in the form of suppositories for rectal or vaginal administration. Suppositories may be prepared by mixing one or more polymer-agent conjugate, particle or composition described herein with one or more suitable non-irritating excipients which is solid at room temperature, but liquid at body temperature. The composition will therefore melt in the rectum or vaginal cavity and release the polymer-agent conjugate, particle or composition. Such materials include, for example, cocoa butter, polyethylene glycol, a suppository wax or a salicylate. Compositions of the present invention which are suitable for vaginal administration also include pessaries, tampons, creams, gels, pastes, foams or spray formulations containing such carriers as are known in the art to be appropriate.

Ophthalmic formulations, eye ointments, powders, solutions and the like, are also contemplated as being within the scope of the invention. An ocular tissue (e.g., a deep cortical region, a supranuclear region, or an aqueous humor region of an eye) may be contacted with the ophthalmic formulation, which is allowed to distribute into the lens. Any suitable method(s) of administration or application of the ophthalmic formulations of the invention (e.g., topical, injection, parenteral, airborne, etc.) may be employed. For example, the contacting may occur via topical administration or via injection.

### Dosages and Dosage Regimens

The polymer-agent conjugate(s), particle(s) or composition(s) can be formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular subject, composition, and mode of administration, without being toxic to the subject.

In one embodiment, the polymer-agent conjugate, particle or composition is administered to a subject at a dosage of, e.g., about 0.1 to 300 mg/m², about 5 to 275 mg/m², about 10 to 250 mg/m², e.g., about 15, 20, 25, 30, 35, 40, 45, 50, 55, 60, 65, 70, 80, 90, 100, 110, 120, 130, 140, 150, 160, 170, 180, 190, 200, 210, 220, 230, 240, 250, 260, 270, 280, 290 mg/m². Administration can be at regular intervals, such as every 1, 2, 3, 4, or 5 days, or weekly, or every 2, 3, 4, 5, 6, or 7 or 8 weeks. The administration can be over a period of from about 10 minutes to about 6 hours, e.g., from about 30 minutes to about 2 hours, from about 45 minutes to 90 minutes, e.g., about 30 minutes, 45 minutes, 1 hour, 2 hours, 3 hours, 4 hours, 5 hours or more. In one embodiment, the polymer-agent conjugate, particle or composition is administered as a bolus infusion or intravenous push, e.g., over a period of 15 minutes, 10 minutes, 5 minutes or less. In one embodiment, the polymer-agent conjugate, particle or composition is administered in an amount such the desired dose of the agent is administered. Preferably the dose of the polymer-agent conjugate, particle or composition is a dose described herein.

In one embodiment, the subject receives 1, 2, 3, up to 10, up to 12, up to 15 treatments, or more, or until the disorder or a symptom of the disorder is cured, healed, alleviated, relieved, altered, remedied, ameliorated, palliated, improved or affected. For example, the subject receive an infusion once every 1, 2, 3 or 4 weeks until the disorder or a symptom of the disorder are cured, healed, alleviated, relieved, altered, remedied, ameliorated, palliated, improved or affected. Preferably, the dosing schedule is a dosing schedule described herein.

The polymer, particle, or composition can be administered as a first line therapy, e.g., alone or in combination with an additional agent or agents. In other embodiments, a polymer-agent conjugate, particle or composition is administered after a subject has developed resistance to, has failed to respond to or has relapsed after a first line therapy. The polymer-agent conjugate, particle or composition may be administered in combination with a second agent. Preferably, the polymer-agent conjugate, particle or composition is administered in combination with a second agent described herein. The second agent may be the same or different as the agent in the particle.

### Kits

A polymer-agent conjugate, particle or composition described herein may be provided in a kit. The kit includes a polymer-agent conjugate, particle or composition described herein and, optionally, a container, a pharmaceutically acceptable carrier and/or informational material. The informational material can be descriptive, instructional, marketing or other material that relates to the methods described herein and/or the use of the particles for the methods described herein.

The informational material of the kits is not limited in its form. In one embodiment, the informational material can include information about production of the polymer-agent conjugate, particle or composition, physical properties of the polymer-agent conjugate, particle or composition, concentration, date of expiration, batch or production site information, and so forth. In one embodiment, the informational material relates to methods for administering the polymer-agent conjugate, particle or composition.

In one embodiment, the informational material can include instructions to administer a polymer-agent conjugate, particle or composition described herein in a suitable manner to perform the methods described herein, e.g., in a suitable dose, dosage form, or mode of administration (e.g., a dose, dosage form, or mode of administration described herein). In another embodiment, the informational material can include instructions to administer a polymer-agent conjugate, particle or composition described herein to a suitable subject, e.g., a human, e.g., a human having or at risk for a disorder described herein. In another embodiment, the informational material can include instructions to reconstitute a polymer-agent conjugate or particle described herein into a pharmaceutically acceptable composition.

In one embodiment, the kit includes instructions to use the polymer-agent conjugate, particle or composition, such as for treatment of a subject. The instructions can include methods for reconstituting or diluting the polymer-agent conjugate, particle or composition for use with a particular subject or in combination with a particular chemotherapeutic agent. The instructions can also include methods for reconstituting or diluting the polymer conjugate composition for use with a particular means of administration, such as by intravenous infusion.

In another embodiment, the kit includes instructions for treating a subject with a particular indication, such as a particular cancer, or a cancer at a particular stage. For example, the instructions can be for a cancer or cancer at stage described herein. The instructions may also address first line treatment of a subject who has a particular cancer, or cancer at a stage described herein. The instructions can also address treatment of a subject who has been non-responsive to a first line therapy or has become sensitive (e.g., has one or more unacceptable side effect) to a first line therapy, such as a taxane, an anthracycline, an alkylating agent, a platinum based agent, a vinca alkaloid. In another embodiment, the instructions will describe treatment of selected subjects with the polymer-agent conjugate, particle or composition. For example, the instructions can describe treatment of one or more of: a subject who has received an anticancer agent (e.g., docetaxel, paclitaxel, larotaxel, cabazitaxel, doxorubicin) and has a neutrophil count less than a standard; a subject who has moderate to severe neutropenia; a subject who has experienced one or more symptom of neuropathy from treatment with an anticancer agent, e.g., a taxane, a vinca alkaloid, an alkylating agent, an anthracycline, a platinum-based agent or an epothilone; a subject who has experienced an infusion site reaction or has or is at risk for having hypersensitivity to treatment with an anticancer agent (e.g., a taxane); a subject having transaminase (ALT and/or AST levels) greater than the upper limit of normal (ULN) and/or bilirubin levels greater than ULN; a subject having ALP levels greater than the upper limit of normal (ULN), SGOT and/or SGPT levels greater the upper limit of normal (ULN) and/or bilirubin levels greater than the ULN; a subject who is currently being administered or will be administered a cytochrome P450 isoenzyme inhibitor; and a subject who has or is at risk for having fluid retention and/or effusion.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about a particle described herein and/or its use in the methods described herein. The informational material can also be provided in any combination of formats.

In addition to a polymer-agent conjugate, particle or composition described herein, the composition of the kit can include other ingredients, such as a surfactant, a lyoprotectant or stabilizer, an antioxidant, an antibacterial agent, a bulking agent, a chelating agent, an inert gas, a tonicity agent and/or a viscosity agent, a solvent or buffer, a stabilizer, a preservative, a flavoring agent (e.g., a bitter antagonist or a sweetener), a fragrance, a dye or coloring agent, for example, to tint or color one or more components in the kit, or other cosmetic ingredient, a pharmaceutically acceptable carrier and/or a second agent for treating a condition or disorder described herein. Alternatively, the other ingredients can be included in the kit, but in different compositions or containers than a particle described herein. In such embodiments, the kit can include instructions for admixing a polymer-agent conjugate, particle or composition described herein and the other ingredients, or for using a polymer-agent conjugate, particle or composition described herein together with the other ingredients.

In another embodiment, the kit includes a second therapeutic agent, such as a second chemotherapeutic agent, e.g., a chemotherapeutic agent or combination of chemotherapeutic agents described herein. In one embodiment, the second agent is in lyophilized or in liquid form. In one embodiment, the polymer-agent conjugate, particle or composition and the second therapeutic agent are in separate containers, and in another embodiment, the polymer-agent conjugate, particle or composition and the second therapeutic agent are packaged in the same container.

In some embodiments, a component of the kit is stored in a sealed vial, e.g., with a rubber or silicone enclosure (e.g., a polybutadiene or polyisoprene enclosure). In some embodiments, a component of the kit is stored under inert conditions (e.g., under Nitrogen or another inert gas such as Argon). In some embodiments, a component of the kit is stored under anhydrous conditions (e.g., with a desiccant). In some embodiments, a component of the kit is stored in a light blocking container such as an amber vial.

A polymer-agent conjugate, particle or composition described herein can be provided in any form, e.g., liquid, frozen, dried or lyophilized form. It is preferred that a polymer-agent conjugate, particle or composition described herein be substantially pure and/or sterile. In an embodiment, the polymer-agent conjugate, particle or composition is sterile. When a polymer-agent conjugate, particle or composition described herein is provided in a liquid solution, the liquid solution preferably is an aqueous solution, with a sterile aqueous solution being preferred. In one embodiment, the polymer-agent conjugate, particle or composition is provided in lyophilized form and, optionally, a diluent solution is provided for reconstituting the lyophilized agent. The diluent can include for example, a salt or saline solution, e.g., a sodium chloride solution having a pH between 6 and 9, lactated Ringer's injection solution, D5W, or PLASMA-LYTE A Injection pH 7.4® (Baxter, Deerfield, Ill.).

The kit can include one or more containers for the composition containing a polymer-agent conjugate, particle or composition described herein. In some embodiments, the kit contains separate containers, dividers or compartments for the composition and informational material. For example, the composition can be contained in a bottle, vial, IV admixture bag, IV infusion set, piggyback set or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of a polymer-agent conjugate, particle or composition described herein. For example, the kit includes a plurality of syringes, ampules, foil packets, or blister packs, each containing a single unit dose of a particle described herein. The containers of the kits can be air tight, waterproof (e.g., impermeable to changes in moisture or evaporation), and/or light-tight.

The kit optionally includes a device suitable for administration of the composition, e.g., a syringe, inhalant, pipette, forceps, measured spoon, dropper (e.g., eye dropper), swab (e.g., a cotton swab or wooden swab), or any such delivery device. In one embodiment, the device is a medical implant device, e.g., packaged for surgical insertion.

### Methods of Using Particles and Compositions

The polymer-agent conjugates, particles and compositions described herein can be administered to cells in culture, e.g. in vitro or ex vivo, or to a subject, e.g., in vivo, to treat or prevent a variety of disorders, including those described herein below. The polymer-agent conjugates, particles and compositions can be used as part of a first line, second line, or adjunct therapy, and can also be used alone or in combination with one or more additional treatment regimes.

### Cancer

The disclosed polymer-agent conjugates, particles and compositions are useful in treating proliferative disorders, e.g., treating a tumor and metastases thereof wherein the tumor or metastases thereof is a cancer described herein.

The methods described herein can be used to treat a solid tumor, a soft tissue tumor or a liquid tumor. Exemplary solid tumors include malignancies (e.g., sarcomas and carcinomas (e.g., adenocarcinoma or squamous cell carcinoma)) of the various organ systems, such as those of brain, lung, breast, lymphoid, gastrointestinal (e.g., colon), and genitourinary (e.g., renal, urothelial, or testicular tumors) tracts, pharynx, prostate, and ovary. Exemplary adenocarcinomas include colorectal cancers, renal-cell carcinoma, liver cancer, non-small cell carcinoma of the lung, and cancer of the small intestine. The disclosed methods are also useful in evaluating or treating soft tissue tumors such as those of the tendons, muscles or fat, and liquid tumors.

The methods described herein can be used with any cancer, for example those described by the National Cancer Institute. The cancer can be a carcinoma, a sarcoma, a myeloma, a leukemia, a lymphoma or a mixed type. Exemplary cancers described by the National Cancer Institute include:

Digestive/gastrointestinal cancers such as anal cancer; bile duct cancer; extrahepatic bile duct cancer; appendix cancer; carcinoid tumor, gastrointestinal cancer; colon cancer; colorectal cancer including childhood colorectal cancer; esophageal cancer including childhood esophageal cancer; gallbladder cancer; gastric (stomach) cancer including childhood gastric (stomach) cancer; hepatocellular (liver) cancer including adult (primary) hepatocellular (liver) cancer and childhood (primary) hepatocellular (liver) cancer; pancreatic cancer including childhood pancreatic cancer; sarcoma, rhabdomyosarcoma; islet cell pancreatic cancer; rectal cancer; and small intestine cancer;

Endocrine cancers such as islet cell carcinoma (endocrine pancreas); adrenocortical carcinoma including childhood adrenocortical carcinoma; gastrointestinal carcinoid tumor; parathyroid cancer; pheochromocytoma; pituitary tumor; thyroid cancer including childhood thyroid cancer; childhood multiple endocrine neoplasia syndrome; and childhood carcinoid tumor;

Eye cancers such as intraocular melanoma; and retinoblastoma;

Musculoskeletal cancers such as Ewing's family of tumors; osteosarcoma/malignant fibrous histiocytoma of the bone; childhood rhabdomyosarcoma; soft tissue sarcoma including adult and childhood soft tissue sarcoma; clear cell sarcoma of tendon sheaths; and uterine sarcoma;

Breast cancer such as breast cancer including childhood and male breast cancer and pregnancy;

Neurologic cancers such as childhood brain stem glioma; brain tumor; childhood cerebellar astrocytoma; childhood cerebral astrocytoma/malignant glioma; childhood ependymoma; childhood medulloblastoma; childhood pineal and supratentorial primitive neuroectodermal tumors; childhood visual pathway and hypothalamic glioma; other childhood brain cancers; adrenocortical carcinoma; central nervous system lymphoma, primary; childhood cerebellar astrocytoma; neuroblastoma; craniopharyngioma; spinal cord tumors; central nervous system atypical teratoid/rhabdoid tumor; central nervous system embryonal tumors; and childhood supratentorial primitive neuroectodermal tumors and pituitary tumor;

Genitourinary cancers such as bladder cancer including childhood bladder cancer; renal cell (kidney) cancer; ovarian cancer including childhood ovarian cancer; ovarian epithelial cancer; ovarian low malignant potential tumor; penile cancer; prostate cancer; renal cell cancer including childhood renal cell cancer; renal pelvis and ureter, transitional cell cancer; testicular cancer; urethral cancer; vaginal cancer; vulvar cancer; cervical cancer; Wilms tumor and other childhood kidney tumors; endometrial cancer; and gestational trophoblastic tumor;

Germ cell cancers such as childhood extracranial germ cell tumor; extragonadal germ cell tumor; ovarian germ cell tumor; and testicular cancer;

Head and neck cancers such as lip and oral cavity cancer; oral cancer including childhood oral cancer; hypopharyngeal cancer; laryngeal cancer including childhood laryngeal cancer; metastatic squamous neck cancer with occult primary; mouth cancer; nasal cavity and paranasal sinus cancer; nasopharyngeal cancer including childhood nasopharyngeal cancer; oropharyngeal cancer; parathyroid cancer; pharyngeal cancer; salivary gland cancer including childhood salivary gland cancer; throat cancer; and thyroid cancer;

Hematologic/blood cell cancers such as a leukemia (e.g., acute lymphoblastic leukemia including adult and childhood acute lymphoblastic leukemia; acute myeloid leukemia including adult and childhood acute myeloid leukemia; chronic lymphocytic leukemia; chronic myelogenous leukemia; and hairy cell leukemia); a lymphoma (e.g., AIDS-related lymphoma; cutaneous T-cell lymphoma; Hodgkin's lymphoma including adult and childhood Hodgkin's lymphoma and Hodgkin's lymphoma during pregnancy; non-Hodgkin's lymphoma including adult and childhood non-Hodgkin's lymphoma and non-Hodgkin's lymphoma during pregnancy; mycosis fungoides; Sézary syndrome; Waldenstrom's macroglobulinemia; and primary central nervous system lymphoma); and other hematologic cancers (e.g., chronic myeloproliferative disorders; multiple myeloma/plasma cell neoplasm; myelodysplastic syndromes; and myelodysplastic/myeloproliferative disorders);

Lung cancer such as non-small cell lung cancer; and small cell lung cancer;

Respiratory cancers such as malignant mesothelioma, adult; malignant mesothelioma, childhood; malignant thymoma; childhood thymoma; thymic carcinoma; bronchial adenomas/carcinoids including childhood bronchial adenomas/carcinoids; pleuropulmonary blastoma; non-small cell lung cancer; and small cell lung cancer;

Skin cancers such as Kaposi's sarcoma; Merkel cell carcinoma; melanoma; and childhood skin cancer;

AIDS-Related Malignancies;

Other childhood cancers, unusual cancers of childhood and cancers of unknown primary site;

and metastases of the aforementioned cancers can also be treated or prevented in accordance with the methods described herein.

The polymer-agent conjugates, compounds or compositions described herein are particularly suited to treat accelerated or metastatic cancers of the bladder cancer, pancreatic cancer, prostate cancer, renal cancer, non-small cell lung cancer, ovarian cancer, melanoma, colorectal cancer, and breast cancer.

In one embodiment, a method is provided for a combination treatment of a cancer, such as by treatment with a polymer-agent conjugate, compound or composition and a second therapeutic agent. Various combinations are described herein. The combination can reduce the development of tumors, reduces tumor burden, or produce tumor regression in a mammalian host.

### Cancer Combination Therapy

The polymer-agent conjugate, compound or composition may be used in combination with other known therapies. Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

The polymer-agent conjugate, compound or composition and the at least one additional agent can be administered simultaneously, in the same or in separate compositions, or sequentially. For sequential administration, the polymer-agent conjugate, compound or composition can be administered first, and the additional agent can be administered second, or the order of administration can be reversed.

In some embodiments, the polymer-agent conjugate, compound or composition is administered in combination with other therapeutic treatment modalities, including surgery, radiation, cryosurgery, and/or thermotherapy. Such combination therapies may advantageously utilize lower dosages of the administered agent and/or other chemotherapeutic agent, thus avoiding possible toxicities or complications associated with the various monotherapies. The phrase “radiation” includes, but is not limited to, external-beam therapy which involves three dimensional, conformal radiation therapy where the field of radiation is designed to conform to the volume of tissue treated; interstitial-radiation therapy where seeds of radioactive compounds are implanted using ultrasound guidance; and a combination of external-beam therapy and interstitial-radiation therapy.

In some embodiments, the polymer-agent conjugate, compound or composition is administered with at least one additional agent, such as a chemotherapeutic agent. In certain embodiments, the polymer-agent conjugate, compound or composition is administered in combination with one or more additional chemotherapeutic agent, e.g., with one or more chemotherapeutic agents described herein.

In some embodiments, the polymer-agent conjugate, compound or composition is administered in combination with a chemotherapeutic agent. Exemplary classes of chemotherapeutic agents include, e.g., the following:

alkylating agents (including, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil Nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexylen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®).

anti-EGFR antibodies (e.g., cetuximab (Erbitux®), panitumumab (Vectibix®), and gefitinib (Iressa®)).

anti-Her-2 antibodies (e.g., trastuzumab (Herceptin®) and other antibodies from Genentech).

antimetabolites (including, without limitation, folic acid antagonists (also referred to herein as antifolates), pyrimidine analogs, purine analogs and adenosine deaminase inhibitors): methotrexate (Rheumatrex®, Trexall®), 5-fluorouracil (Adrucil®, Efudex®, Fluoroplex®), floxuridine (FUDF®), cytarabine (Cytosar-U®, Tarabine PFS), 6-mercaptopurine (Puri-Nethol®)), 6-thioguanine (Thioguanine Tabloid®), fludarabine phosphate (Fludara®), pentostatin (Nipent®), pemetrexed (Alimta®), raltitrexed (Tomudex®), cladribine (Leustatin®), clofarabine (Clofarex®, Clolar®), mercaptopurine (Puri-Nethol®), capecitabine (Xeloda®), nelarabine (Arranon®), azacitidine (Vidaza®) and gemcitabine (Gemzar®). Preferred antimetabolites include, e.g., 5-fluorouracil (Adrucil®, Efudex®, Fluoroplex®), floxuridine (FUDF®), capecitabine (Xeloda®), pemetrexed (Alimta®), raltitrexed (Tomudex®) and gemcitabine (Gemzar®).

vinca alkaloids: vinblastine (Velban®, Velsar®), vincristine (Vincasar®, Oncovin®), vindesine (Eldisine®), vinorelbine (Navelbine®).

platinum-based agents: carboplatin (Paraplat®, Paraplatin®), cisplatin (Platinol®), oxaliplatin (Eloxatin®).

anthracyclines: daunorubicin (Cerubidine®, Rubidomycin®), doxorubicin (Adriamycin®), epirubicin (Ellence®), idarubicin (Idamycin®), mitoxantrone (Novantrone®), valrubicin (Valstar®). Preferred anthracyclines include daunorubicin (Cerubidine®, Rubidomycin®) and doxorubicin (Adriamycin®).

topoisomerase inhibitors: topotecan (Hycamtin®), irinotecan (Camptosar®), etoposide (Toposar®, VePesid®), teniposide (Vumon®), lamellarin D, SN-38, camptothecin (e.g., IT-101).

taxanes: paclitaxel (Taxol®), docetaxel (Taxotere®), larotaxel, cabazitaxel.

epothilones: ixabepilone, epothilone B, epothilone D, BMS310705, dehydelone, ZK-Epothilone (ZK-EPO).

antibiotics: actinomycin (Cosmegen®), bleomycin (Blenoxane®), hydroxyurea (Droxia®, Hydrea®), mitomycin (Mitozytrex®, Mutamycin®).

immunomodulators: lenalidomide (Revlimid®), thalidomide (Thalomid®).

immune cell antibodies: alemtuzamab (Campath®), gemtuzumab (Myelotarg®), rituximab (Rituxan®), tositumomab (Bexxar®).

interferons (e.g., IFN-alpha (Alferon®, Roferon-A® Intron®-A) or IFN-gamma (Actimmune®))

interleukins: IL-1, IL-2 (Proleukin®), IL-24, IL-6 (Sigosix®), IL-12.

HSP90 inhibitors (e.g., geldanamycin or any of its derivatives). In certain embodiments, the HSP90 inhibitor is selected from geldanamycin, 17-alkylamino-17-desmethoxygeldanamycin (“17-AAG”) or 17-(2-dimethylaminoethyl)amino-17-desmethoxygeldanamycin (“17-DMAG”).

anti-androgens which include, without limitation nilutamide (Nilandron®) and bicalutamide (Caxodex®).

antiestrogens which include, without limitation tamoxifen (Nolvadex®), toremifene (Fareston®), letrozole (Femara®), testolactone (Teslac®), anastrozole (Arimidex®), bicalutamide (Casodex®), exemestane (Aromasin®), flutamide (Eulexin®), fulvestrant (Faslodex®), raloxifene (Evista®, Keoxifene®) and raloxifene hydrochloride.

anti-hypercalcaemia agents which include without limitation gallium (III) nitrate hydrate (Ganite®) and pamidronate disodium (Aredia®).

apoptosis inducers which include without limitation ethanol, 2-[[3-(2,3-dichlorophenoxy)propyl]amino]-(9Cl), gambogic acid, embelin and arsenic trioxide (Trisenox®).

Aurora kinase inhibitors which include without limitation binucleine 2.

Bruton's tyrosine kinase inhibitors which include without limitation terreic acid.

calcineurin inhibitors which include without limitation cypermethrin, deltamethrin, fenvalerate and tyrphostin 8.

CaM kinase II inhibitors which include without limitation 5-Isoquinolinesulfonic acid, 4-[{2S)-2-[(5-isoquinolinylsulfonyl)methylamino]-3-oxo-3-{4-phenyl-1-piperazinyl)propyl]phenyl ester and benzenesulfonamide.

CD45 tyrosine phosphatase inhibitors which include without limitation phosphonic acid.

CDC25 phosphatase inhibitors which include without limitation 1,4-naphthalene dione, 2,3-bis[(2-hydroxyethyl)thio]-(9Cl).

CHK kinase inhibitors which include without limitation debromohymenialdisine.

cyclooxygenase inhibitors which include without limitation 1H-indole-3-acetamide, 1-(4-chlorobenzoyl)-5-methoxy-2-methyl-N-(2-phenylethyl)-(9Cl), 5-alkyl substituted 2-arylaminophenylacetic acid and its derivatives (e.g., celecoxib (Celebrex®), rofecoxib (Vioxx®), etoricoxib (Arcoxia®), lumiracoxib (Prexige®), valdecoxib (Bextra®) or 5-alkyl-2-arylaminophenylacetic acid).

cRAF kinase inhibitors which include without limitation 3-(3,5-dibromo-4-hydroxybenzylidene)-5-iodo-1,3-dihydroindol-2-one and benzamide, 3-(dimethylamino)-N-[3-[(4-hydroxybenzoyl)amino]-4-methylphenyl]-(9Cl).

cyclin dependent kinase inhibitors which include without limitation olomoucine and its derivatives, purvalanol B, roascovitine (Seliciclib®), indirubin, kenpaullone, purvalanol A and indirubin-3′-monooxime.

cysteine protease inhibitors which include without limitation 4-morpholinecarboxamide, N-[(1S)-3-fluoro-2-oxo-1-(2-phenylethyl)propyl]amino]-2-oxo-1-(phenylmethyl)ethyl]-(9Cl).

DNA intercalators which include without limitation plicamycin (Mithracin®) and daptomycin (Cubicin®).

DNA strand breakers which include without limitation bleomycin (Blenoxane®).

E3 ligase inhibitors which include without limitation N-((3,3,3-trifluoro-2-trifluoromethyl)propionyl)sulfanilamide.

EGF Pathway Inhibitors which include, without limitation tyrphostin 46, EKB-569, erlotinib (Tarceva®), gefitinib (Iressa®), lapatinib (Tykerb®) and those compounds that are generically and specifically disclosed in WO 97/02266, EP 0 564 409, WO 99/03854, EP 0 520 722, EP 0 566 226, EP 0 787 722, EP 0 837 063, U.S. Pat. No. 5,747,498, WO 98/10767, WO 97/30034, WO 97/49688, WO 97/38983 and WO 96/33980.

farnesyltransferase inhibitors which include without limitation A-hydroxyfarnesylphosphonic acid, butanoic acid, 2-[(2S)-2-[[(2S,3S)-2-[[(2R)-2-amino-3-mercaptopropyl]amino]-3-methylpentyl]oxy]-1-oxo-3-phenylpropyl]amino]-4-(methylsulfonyl)-1-methylethylester (2S)-(9Cl), and manumycin A.

Flk-1 kinase inhibitors which include without limitation 2-propenamide, 2-cyano-3-[4-hydroxy-3,5-bis(1-methylethyl)phenyl]-N-(3-phenylpropyl)-(2E)-(9Cl).

glycogen synthase kinase-3 (GSK3) inhibitors which include without limitation indirubin-3′-monooxime.

histone deacetylase (HDAC) inhibitors which include without limitation suberoylanilide hydroxamic acid (SAHA), [4-(2-amino-phenylcarbamoyl)-benzyl]-carbamic acid pyridine-3-ylmethylester and its derivatives, butyric acid, pyroxamide, trichostatin A, oxamflatin, apicidin, depsipeptide, depudecin, trapoxin and compounds disclosed in WO 02/22577.

I-kappa B-alpha kinase inhibitors (IKK) which include without limitation 2-propenenitrile, 3-[(4-methylphenyl)sulfonyl]-(2E)-(9Cl).

imidazotetrazinones which include without limitation temozolomide (Methazolastone®, Temodar® and its derivatives (e.g., as disclosed generically and specifically in U.S. Pat. No. 5,260,291) and Mitozolomide.

insulin tyrosine kinase inhibitors which include without limitation hydroxyl-2-naphthalenylmethylphosphonic acid.

c-Jun-N-terminal kinase (JNK) inhibitors which include without limitation pyrazoleanthrone and epigallocatechin gallate.

mitogen-activated protein kinase (MAP) inhibitors which include without limitation benzenesulfonamide, N-[2-[[[3-(4-chlorophenyl)-2-propenyl]methyl]amino]methyl]phenyl]-N-(2-hydroxyethyl)-4-methoxy-(9Cl).

MDM2 inhibitors which include without limitation trans-4-iodo, 4′-boranyl-chalcone.

MEK inhibitors which include without limitation butanedinitrile, bis[amino[2-aminophenyl)thio]methylene]-(9Cl).

MMP inhibitors which include without limitation Actinonin, epigallocatechin gallate, collagen peptidomimetic and non-peptidomimetic inhibitors, tetracycline derivatives marimastat (Marimastat®), prinomastat, incyclinide (Metastat®), shark cartilage extract AE-941 (Neovastat®), Tanomastat, TAA211, MMI270B or AAJ996.

mTor inhibitors which include without limitation rapamycin (Rapamune®), and analogs and derivatives thereof, AP23573 (also known as ridaforolimus, deforolimus, or MK-8669), CCI-779 (also known as temsirolimus) (Torisel®) and SDZ-RAD.

NGFR tyrosine kinase inhibitors which include without limitation tyrphostin AG 879.

p38 MAP kinase inhibitors which include without limitation Phenol, 4-[4-(4-fluorophenyl)-5-(4-pyridinyl)-1H-imidazol-2-yl]-(9Cl), and benzamide, 3-(dimethylamino)-N-[3-[(4-hydroxylbenzoyl)amino]-4-methylphenyl]-(9Cl).

p56 tyrosine kinase inhibitors which include without limitation damnacanthal and tyrphostin 46.

PDGF pathway inhibitors which include without limitation tyrphostin AG 1296, tyrphostin 9, 1,3-butadiene-1,1,3-tricarbonitrile, 2-amino-4-(1H-indol-5-yl)-(9Cl), imatinib (Gleevec®) and gefitinib (Iressa®) and those compounds generically and specifically disclosed in European Patent No.: 0 564 409 and PCT Publication No.: WO 99/03854.

phosphatidylinositol 3-kinase inhibitors which include without limitation wortmannin, and quercetin dihydrate.

phosphatase inhibitors which include without limitation cantharidic acid, cantharidin, and L-leucinamide.

protein phosphatase inhibitors which include without limitation cantharidic acid, cantharidin, L-P-bromotetramisole oxalate, 2(5H)-furanone, 4-hydroxy-5-(hydroxymethyl)-3-(1-oxohexadecyl)-(5R)-(9Cl) and benzylphosphonic acid.

PKC inhibitors which include without limitation 1-H-pyrollo-2,5-dione,3-[1-[3-(dimethylamino)propyl]-1H-indol-3-yl]-4-(1H-indol-3-yl)-(9Cl), Bisindolylmaleimide IX, Sphinogosine, staurosporine, and Hypericin.

PKC delta kinase inhibitors which include without limitation rottlerin.

polyamine synthesis inhibitors which include without limitation DMFO.

proteasome inhibitors which include, without limitation aclacinomycin A, gliotoxin and bortezomib (Velcade®).

PTP1B inhibitors which include without limitation L-leucinamide.

protein tyrosine kinase inhibitors which include, without limitation tyrphostin Ag 216, tyrphostin Ag 1288, tyrphostin Ag 1295, geldanamycin, genistein and 7H-pyrollo[2,3-d]pyrimidine derivatives as generically and specifically described in PCT Publication No.: WO 03/013541 and U.S. Publication No.: 2008/0139587.

SRC family tyrosine kinase inhibitors which include without limitation PP1 and PP2.

Syk tyrosine kinase inhibitors which include without limitation piceatannol.

Janus (JAK-2 and/or JAK-3) tyrosine kinase inhibitors which include without limitation tyrphostin AG 490 and 2-naphthyl vinyl ketone.

retinoids which include without limitation isotretinoin (Accutane®, Amnesteem®, Cistane®, Claravis®, Sotret®) and tretinoin (Aberel®, Aknoten®, Avita®, Renova®, Retin-A®, Retin-A MICRO®, Vesanoid®).

RNA polymerase II elongation inhibitors which include without limitation 5,6-dichloro-1-beta-D-ribofuranosylbenzimidazole.

serine/Threonine kinase inhibitors which include without limitation 2-aminopurine.

sterol biosynthesis inhibitors which include without limitation squalene epoxidase and CYP2D6.

VEGF pathway inhibitors, which include without limitation anti-VEGF antibodies, e.g., bevacizumab, and small molecules, e.g., sunitinib (Sutent®), sorafinib (Nexavar®), ZD6474 (also known as vandetanib) (Zactima™), SU6668, CP-547632 and AZD2171 (also known as cediranib) (Recentin™).

Examples of chemotherapeutic agents are also described in the scientific and patent literature, see, e.g., Bulinski (1997) J. Cell Sci. 110:3055-3064; Panda (1997) Proc. Natl. Acad. Sci. USA 94:10560-10564; Muhlradt (1997) Cancer Res. 57:3344-3346; Nicolaou (1997) Nature 387:268-272; Vasquez (1997) Mol. Biol. Cell. 8:973-985; Panda (1996) J. Biol. Chem. 271:29807-29812.

In some embodiments, the polymer-agent conjugate, compound or composition is administered instead of another microtubule affecting agent, e.g., instead of a microtubule affecting agent as a first line therapy or a second line therapy. For example, the polymer-agent conjugate, compound or composition can be used instead of any of the following microtubule affecting agents allocolchicine (NSC 406042), halichondrin B (NSC 609395), colchicine (NSC 757), colchicine derivatives (e.g., NSC 33410), dolastatin 10 (NSC 376128), maytansine (NSC 153858), rhizoxin (NSC 332598), paclitaxel (Taxol®, NSC 125973), taxol derivatives (e.g., derivatives (e.g., NSC 608832), thiocolchicine (NSC 361792), trityl cysteine (NSC 83265), vinblastine sulfate (NSC 49842), vincristine sulfate (NSC 67574).

In some cases, a hormone and/or steroid can be administered in combination with a polymer-agent conjugate, compound or composition. Examples of hormones and steroids include: 17a-ethinylestradiol (Estinyl®, Ethinoral®, Feminone®, Orestralyn®), diethylstilbestrol (Acnestrol®, Cyren A®, Deladumone®, Diastyl®, Domestrol®, Estrobene®, Estrobene®, Estrosyn®, Fonatol®, Makarol®, Milestrol®, Milestrol®, Neo-Oestronol I®, Oestrogenine®, Oestromenin®, Oestromon®, Palestrol®, Stilbestrol®, Stilbetin®, Stilboestroform®, Stilboestrol®, Synestrin®, Synthoestrin®, Vagestrol®), testosterone (Delatestryl®, Testoderm®, Testolin®, Testostroval®, Testostroval-PA®, Testro AQ®), prednisone (Delta-Dome®, Deltasone®, Liquid Pred®, Lisacort®, Meticorten®, Orasone®, Prednicen-M®, Sk-Prednisone®, Sterapred®), Fluoxymesterone (Android-F®, Halodrin®, Halotestin®, Ora-Testryl®, Ultandren®), dromostanolone propionate (Drolban®, Emdisterone®, Masterid®, Masteril®, Masteron®, Masterone®, Metholone®, Permastril®), testolactone (Teslac®), megestrolacetate (Magestin®, Maygace®, Megace®, Megeron®, Megestat®, Megestil®, Megestin®, Nia®, Niagestin®, Ovaban®, Ovarid®, Volidan®), methylprednisolone (Depo-Medrol®, Medlone 21®, Medrol®, Meprolone®, Metrocort®, Metypred®, Solu-Medrol®, Summicort®), methyl-testosterone (Android®, Testred®, Virilon®), prednisolone (Cortalone®, Delta-Cortef®, Hydeltra®, Hydeltrasol®, Meti-derm®, Prelone®), triamcinolone (Aristocort®), chlorotrianisene (Anisene®, Chlorotrisin®, Clorestrolo®, Clorotrisin®, Hormonisene®, Khlortrianizen®, Merbentul®, Metace®, Rianil®, Tace®, Tace-Fn®, Trianisestrol®), hydroxyprogesterone (Delalutin®, Gestiva™), aminoglutethimide (Cytadren®, Elipten®, Orimeten®), estramustine (Emcyt®), medroxyprogesteroneacetate (Provera®, Depo-Provera®), leuprolide (Lupron®, Viadur®), flutamide (Eulexin®), toremifene (Fareston®), and goserelin (Zoladex®).

In certain embodiments, the polymer-agent conjugate, compound or composition is administered in combination with an anti-microbial (e.g., leptomycin B).

In another embodiment, the polymer-agent conjugate, compound or composition is administered in combination with an agent or procedure to mitigate potential side effects from the agent compositions such as diarrhea, nausea and vomiting.

Diarrhea may be treated with antidiarrheal agents including, but not limited to opioids (e.g., codeine (Codicept®, Coducept®), oxicodeine, percocet, paregoric, tincture of opium, diphenoxylate (Lomotil®), diflenoxin), and loperamide (Imodium A-D®), bismuth subsalicylate, lanreotide, vapreotide (Sanvar®, Sanvar IC®), motiln antagonists, COX2 inhibitors (e.g., celecoxib (Celebrex®), glutamine (NutreStore®), thalidomide (Synovir®, Thalomid®), traditional antidiarrhea remedies (e.g., kaolin, pectin, berberine and muscarinic agents), octreotide and DPP-IV inhibitors.

DPP-IV inhibitors employed in the present invention are generically and specifically disclosed in PCT Publication Nos.: WO 98/19998, DE 196 16 486 A1, WO 00/34241 and WO 95/15309.

Nausea and vomiting may be treated with antiemetic agents such as dexamethasone (Aeroseb-Dex®, Alba-Dex®, Decaderm®, Decadrol®, Decadron®, Decasone®, Decaspray®, Deenar®, Deronil®, Dex-4®, Dexace®, Dexameth®, Dezone®, Gammacorten®, Hexadrol®, Maxidex®, Sk-Dexamethasone®), metoclopramide (Reglan®), diphenylhydramine (Benadryl®, SK-Diphenhydramine®), lorazepam (Ativan®), ondansetron (Zofran®), proclorperazine (Bayer A 173®, Buccastem®, Capazine®, Combid®, Compazine®, Compro®, Emelent®, Emetiral®, Eskatrol®, Kronocin®, Meterazin®, Meterazin Maleate®, Meterazine®, Nipodal®, Novamin®, Pasotomin®, Phenotil®, Stemetil®, Stemzine®, Tementil®, Temetid®, Vertigon®), thiethylperazine (Norzine®, Torecan®), and dronabinol (Marinol®).

In some embodiments, the polymer-agent conjugate, compound or composition is administered in combination with an immunosuppressive agent. Immunosuppressive agents suitable for the combination include, but are not limited to natalizumab (Tysabri®), azathioprine (Imuran®), mitoxantrone (Novantrone®), mycophenolate mofetil (Cellcept®), cyclosporins (e.g., Cyclosporin A (Neoral®, Sandimmun®, Sandimmune®, SangCya®), calcineurin inhibitors (e.g., Tacrolimus (Prograf®, Protopic®), sirolimus (Rapamune®), everolimus (Afinitor®), cyclophosphamide (Clafen®, Cytoxan®, Neosar®), or methotrexate (Abitrexate®, Folex®, Methotrexate®, Mexate®)), fingolimod, mycophenolate mofetil (CellCept®), mycophenolic acid (Myfortic®), anti-CD3 antibody, anti-CD25 antibody (e.g., Basiliximab (Simulect®) or daclizumab (Zenapax®)), and anti-TNFα antibody (e.g., Infliximab (Remicade®) or adalimumab (Humira®)).

In some embodiments, a polymer-agent conjugate, compound or composition is administered in combination with a CYP3A4 inhibitor (e.g., ketoconazole (Nizoral®, Xolegel®), itraconazole (Sporanox®), clarithromycin (Biaxin®), atazanavir (Reyataz®), nefazodone (Serzone®, Nefadar®), saquinavir (Invirase®), telithromycin (Ketek®), ritonavir (Norvir®), amprenavir (also known as Agenerase, a prodrug version is fosamprenavir (Lexiva®, Telzir®), indinavir (Crixivan®), nelfinavir (Viracept®), delavirdine (Rescriptor®) or voriconazole (Vfend®)).

When employing the methods or compositions, other agents used in the modulation of tumor growth or metastasis in a clinical setting, such as antiemetics, can also be administered as desired.

Exemplary chemotherapeutic agents that may be administered in combination with a polymer-agent conjugate, compound or composition include, bevacizumab (Avastin®), cisplatin (Platinol®), carboplatin (Paraplat®, Paraplatin®), irinotecan (Camptosar®), floxuridine (FUDF®), 5-fluorouracil (5FU) (Adrucil®, Efudex®, Fluoroplex®), leucovorin (Wellcovorin®), capecitabine (Xeloda®), gemcitabine (Gemzar®), oxaliplatin (Eloxatin®), mitoxantrone (Novantrone®), prednisone (Delta-Dome®, Deltasone®, Liquid Pred®, Lisacort®, Meticorten®, Orasone®, Prednicen-M®, Sk-Prednisone®, Sterapred®), estramustine (Emcyt®), sunitinib (Sutent®), temsirolimus (Torisel®), sorafenib (Nexavar®), everolimus (Afinitor®), cetuximab (Erbitux®), pemetrexed (ALIMTA®), erlotinib (Tarceva®), daunorubicin (Cerubidine®, Rubidomycin®), doxorubicin (Adriamycin®), trastuzumab (Herceptin®), or tamoxifen (Nolvadex®). Exemplary combinations of agents that can be administered with a polymer-agent conjugate, compound or composition include, e.g., bevacizumab (Avastin®) and interferon; 5FU (Adrucil®, Efudex®, Fluoroplex®) and leucovorin (Wellcovorin®); UFT (Uftoral®) and Leucovorin (Wellcovorin®); cisplatin (Platinol®) and pemetrexed (ALIMTA®); cisplastin (Platinol®) and vinorelbine (Navelbine®); cisplastin (Platinol®) and gemcitabine (Gemzar®); cisplastin (Platinol®) and vinblastine (Velban®) Velsar®); cisplastin (Platinol®), dacarbazine (DTIC-Dome®) and vinblastine (Velban®) Velsar®); cisplastin (Platinol®), temozolomide (Methazolastone®, Temodar®) and vinblastine (Velban®) Velsar®); cisplatin (Platinol®) and 5FU (Adrucil®) Efudex®, Fluoroplex®); oxaliplatin (Eloxatin®) and irinotecan (Camptosar®); 5FU (Adrucil®) Efudex®, Fluoroplex®), irinotecan (Camptosar®), and leucovorin (Wellcovorin®); 5FU (Adrucil®) Efudex®, Fluoroplex®), irinotecan (Camptosar®), oxaliplatin (Eloxatin®), and leucovorin (Wellcovorin®); 5FU (Adrucil®, Efudex®, Fluoroplex®) and radiation; 5FU (Adrucil®, Efudex®, Fluoroplex®), radiation and cisplatin (Platinol®); oxaliplatin (Eloxatin®), 5FU (Adrucil®, Efudex®, Fluoroplex®), and leucovorin (Wellcovorin®); capecitabine (Xeloda®), oxaliplatin (Eloxatin®), and bevacizumab (Avastin®); capecitabine (Xeloda®), irinotecan (Camptosar®), and bevacizumab (Avastin®); capecitabine (Xeloda®) and bevacizumab (Avastin®); irinotecan (Camptosar®) and bevacizumab (Avastin®); cetuximab (Erbutux®) and bevacizumab (Avastin®); cetuximab (Erbutux®), irinotecan (Camptosar®) and bevacizumab (Avastin®); panitumumab (Vectibix®) and bevacizumab (Avastin®); 5FU (Adrucil®, Efudex®, Fluoroplex®), leucovorin (Wellcovorin®) and bevacizumab (Avastin®); 5FU (Adrucil®, Efudex®, Fluoroplex®), leucovorin (Wellcovorin®), oxaliplatin (Eloxatin®) and bevacizumab (Avastin®); 5FU (Adrucil®, Efudex®, Fluoroplex®), leucovorin (Wellcovorin®), irinotecan (Camptosar®) and bevacizumab (Avastin®); 5FU (Adrucil®, Efudex®, Fluoroplex®), oxaliplatin (Eloxatin®), irinotecan (Camptosar®), leucovorin (Wellcovorin®) and bevacizumab (Avastin®); and UFT (Uftoral®), irinotecan (Camptosar®) and leucovorin (Wellcovorin®).

When formulating the pharmaceutical compositions featured in the invention the clinician may utilize preferred dosages as warranted by the condition of the subject being treated. For example, in one embodiment, a polymer-agent conjugate, compound or composition may be administered at a dosing schedule described herein, e.g., once every one, two three four, five, or six weeks.

Also, in general, a polymer-agent conjugate, compound or composition, and an additional chemotherapeutic agent(s) do not have to be administered in the same pharmaceutical composition, and may, because of different physical and chemical characteristics, have to be administered by different routes. For example, the polymer-agent conjugate, compound or composition may be administered intravenously while the chemotherapeutic agent(s) may be administered orally. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

In one embodiment, a polymer-agent conjugate, compound or composition is administered once every three weeks and an additional therapeutic agent (or additional therapeutic agents) may also be administered every three weeks for as long as treatment is required. Examples of other chemotherapeutic agents which are administered one every three weeks include: an antimetabolite (e.g., floxuridine (FUDF®), pemetrexed (ALIMTA®), 5FU (Adrucil®, Efudex®, Fluoroplex®)); an anthracycline (e.g., daunorubicin (Cerubidine®, Rubidomycin®), epirubicin (Ellence®), idarubicin (Idamycin®), mitoxantrone (Novantrone®), valrubicin (Valstar®)); a vinca alkaloid (e.g., vinblastine (Velban®, Velsar®), vincristine (Vincasar®, Oncovin®), vindesine (Eldisine®) and vinorelbine (Navelbine®)); a topoisomerase inhibitor (e.g., topotecan (Hycamtin®), irinotecan (Camptosar®), etoposide (Toposar®, VePesid®), teniposide (Vumon®), lamellarin D, SN-38, camptothecin (e.g., IT-101)); and a platinum-based agent (e.g., cisplatin (Platinol®), carboplatin (Paraplat®, Paraplatin®), oxaliplatin (Eloxatin®)).

In another embodiment, the polymer-agent conjugate, compound or composition is administered once every two weeks in combination with one or more additional chemotherapeutic agent that is administered orally. For example, the polymer-agent conjugate, compound or composition can be administered once every two weeks in combination with one or more of the following chemotherapeutic agents: capecitabine (Xeloda®), estramustine (Emcyt®), erlotinib (Tarceva®), rapamycin (Rapamune®), SDZ-RAD, CP-547632; AZD2171, sunitinib (Sutent®), sorafenib (Nexavar®) and everolimus (Afinitor®).

The actual dosage of the polymer-agent conjugate, compound or composition and/or any additional chemotherapeutic agent employed may be varied depending upon the requirements of the subject and the severity of the condition being treated. Determination of the proper dosage for a particular situation is within the skill of the art. Generally, treatment is initiated with smaller dosages which are less than the optimum dose of the compound. Thereafter, the dosage is increased by small amounts until the optimum effect under the circumstances is reached.

In one embodiment, the polymer-agent conjugate, compound or composition can be administered at a dose that includes 0.5 to 300 mg/m² of an agent, e.g., 2.5 mg/m² to 30 mg/m², 9 to 280 mg/m², 0.5 to 100 mg/m², 0.5 to 35 mg/m², 25 to 90 mg/m². Preferably, the polymer-agent conjugate, compound or composition is administered at a dosage described herein.

In some embodiments, when a polymer-agent conjugate, compound or composition is administered in combination with one or more additional chemotherapeutic agent, the additional chemotherapeutic agent (or agents) is administered at a standard dose. For example, a standard dosage for cisplatin is 75-120 mg/m² administered every three weeks; a standard dosage for carboplatin is within the range of 200-600 mg/m² or an AUC of 0.5-8 mg/ml×min; e.g., at an AUC of 4-6 mg/ml×min; a standard dosage for irinotecan is within 100-125 mg/m², once a week; a standard dosage for gemcitabine is within the range of 80-1500 mg/m² administered weekly; a standard dose for UFT is within a range of 300-400 mg/m² per day when combined with leucovorin administration; a standard dosage for leucovorin is 10-600 mg/m² administered weekly.

The disclosure also encompasses a method for the synergistic treatment of cancer wherein a polymer-agent conjugate, compound or composition is administered in combination with an additional chemotherapeutic agent or agents.

The particular choice of polymer conjugate and anti-proliferative cytotoxic agent(s) or radiation will depend upon the diagnosis of the attending physicians and their judgment of the condition of the subject and the appropriate treatment protocol.

If the polymer-agent conjugate, compound or composition and the chemotherapeutic agent(s) and/or radiation are not administered simultaneously or essentially simultaneously, then the initial order of administration of the polymer-agent conjugate, compound or composition, and the chemotherapeutic agent(s) and/or radiation, may be varied. Thus, for example, the polymer-agent conjugate, compound or composition may be administered first followed by the administration of the chemotherapeutic agent(s) and/or radiation; or the chemotherapeutic agent(s) and/or radiation may be administered first followed by the administration of the polymer-agent conjugate, compound or composition. This alternate administration may be repeated during a single treatment protocol. The determination of the order of administration, and the number of repetitions of administration of each therapeutic agent during a treatment protocol, is well within the knowledge of the skilled physician after evaluation of the disease being treated and the condition of the subject.

Thus, in accordance with experience and knowledge, the practicing physician can modify each protocol for the administration of a component (polymer-agent conjugate, compound or composition, anti-neoplastic agent(s), or radiation) of the treatment according to the individual subject's needs, as the treatment proceeds.

The attending clinician, in judging whether treatment is effective at the dosage administered, will consider the general well-being of the subject as well as more definite signs such as relief of disease-related symptoms, inhibition of tumor growth, actual shrinkage of the tumor, or inhibition of metastasis. Size of the tumor can be measured by standard methods such as radiological studies, e.g., CAT or MRI scan, and successive measurements can be used to judge whether or not growth of the tumor has been retarded or even reversed. Relief of disease-related symptoms such as pain, and improvement in overall condition can also be used to help judge effectiveness of treatment.

Neurological Deficits

The disclosed methods can be use to treat neurological deficits due to neurodegeneration in the brain of a subject, e.g., a human subject. The method can include administering a polymer-agent, particle or composition described herein to the subject. As used herein, the phrase “neurological deficits” includes an impairment or absence of a normal neurological function or presence of an abnormal neurological function. Neurodegeneration of the brain can be the result of disease, injury, and/or aging. As used herein, neurodegeneration includes morphological and/or functional abnormality of a neural cell or a population of neural cells. Non-limiting examples of morphological and functional abnormalities include physical deterioration and/or death of neural cells, abnormal growth patterns of neural cells, abnormalities in the physical connection between neural cells, under- or over production of a substance or substances, e.g., a neurotransmitter, by neural cells, failure of neural cells to produce a substance or substances which it normally produces, production of substances, e.g., neurotransmitters, and/or transmission of electrical impulses in abnormal patterns or at abnormal times. Neurodegeneration can occur in any area of the brain of a subject and is seen with many disorders including, for example, head trauma, stroke, ALS, multiple sclerosis, Huntington's disease, Parkinson's disease, and Alzheimer's disease.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

### Example 1

### Purification and Characterization of 5050 PLGA

Step A: A 3-L round-bottom flask equipped with a mechanical stirrer was charged with 5050PLGA (300 g, Mw: 7.8 KDa; Mn: 2.7 KDa) and acetone (900 mL). The mixture was stirred for 1 h at ambient temperature to form a clear yellowish solution.
Step B: A 22-L jacket reactor with a bottom-outlet valve equipped with a mechanical stirrer was charged with MTBE (9.0 L, 30 vol. to the mass of 5050 PLGA). Celite® (795 g) was added to the solution with overhead stirring at ˜200 rpm to produce a suspension. To this suspension was slowly added the solution from Step A over 1 h. The mixture was agitated for an additional one hour after addition of the polymer solution and filtered through a polypropylene filter. The filter cake was washed with MTBE (3×300 mL), conditioned for 0.5 h, air-dried at ambient temperature (typically 12 h) until residual MTBE was ≦5 wt % (as determined by 1H NMR analysis.
Step C: A 12-L jacket reactor with a bottom-outlet valve equipped with a mechanical stirrer was charged with acetone (2.1 L, 7 vol. to the mass of 5050 PLGA). The polymer/Celite® complex from Step B was charged into the reactor with overhead stirring at ˜200 rpm to produce a suspension. The suspension was stirred at ambient temperature for an additional 1 h and filtered through a polypropylene filter. The filter cake was washed with acetone (3×300 mL) and the combined filtrates were clarified through a 0.45 mM in-line filter to produce a clear solution. This solution was concentrated to ˜1000 mL.
Step D: A 22-L jacket reactor with a bottom-outlet valve equipped with a mechanical stirrer was charged with water (9.0 L, 30 vol.) and was cooled down to 0-5° C. using a chiller. The solution from Step C was slowly added over 2 h with overhead stirring at ˜200 rpm. The mixture was stirred for an additional one hour after addition of the solution and filtered through a polypropylene filter. The filter cake was conditioned for 1 h, air-dried for 1 day at ambient temperature, and then vacuum-dried for 3 days to produce the purified 5050 PLGA as a white powder [258 g, 86%]. The ¹H NMR analysis was consistent with that of the desired product and Karl Fisher analysis showed 0.52 wt % of water. The product was analyzed by HPLC (AUC, 230 nm) and GPC (AUC, 230 nm). The process produced a more narrow polymer polydispersity, i.e. Mw: 8.8 kDa and Mn: 5.8 kDa.

### Example 2

### Purification and Characterization of 5050 PLGA Lauryl Ester

A 12-L round-bottom flask equipped with a mechanical stirrer was charged with MTBE (4 L) and heptanes (0.8 L). The mixture was agitated at ˜300 rpm, to which a solution of 5050 PLGA lauryl ester (65 g) in acetone (300 mL) was added dropwise. Gummy solids were formed over time and finally clumped up on the bottom of the flask. The supernatant was decanted off and the solid was dried under vacuum at 25° C. for 24 h to afford 40 g of purified 5050 PLGA lauryl ester as a white powder [yield: 61.5%]. ¹H NMR (CDCl₃, 300 MHz): δ 5.25-5.16 (m, 53H), 4.86-4.68 (m, 93H), 4.18 (m, 7H), 1.69-1.50 (m, 179H), 1.26 (bs, 37H), 0.88 (t, J=6.9 Hz, 6H). The ¹H NMR analysis was consistent with that of the desired product. GPC (AUC, 230 nm): 6.02-9.9 min, t_{R}=7.91 min.

### Example 3

### Purification and Characterization of 7525 PLGA

A 22-L round-bottom flask equipped with a mechanical stirrer was charged with 12 L of MTBE, to which a solution of 7525 PLGA (150 g, approximately 6.6 kD) in dichloromethane (DCM, 750 mL) was added dropwise over an hour with an agitation of ˜300 rpm, resulting in a gummy solid. The supernatant was decanted off and the gummy solid was dissolved in DCM (3 L). The solution was transferred to a round-bottom flask and concentrated to a residue, which was dried under vacuum at 25° C. for 40 h to afford 94 g of purified 7525 PLGA as a white foam [yield: 62.7%,]. ¹H NMR (CDCl₃, 300 MHz): δ 5.24-5.15 (m, 68H), 4.91-4.68 (m, 56H), 3.22 (s, 2.3H, MTBE), 1.60-1.55 (m, 206H), 1.19 (s, 6.6H, MTBE). The ¹H NMR analysis was consistent with that of the desired product. GPC (AUC, 230 nm): 6.02-9.9 min, t_{R}=7.37 min.

### Example 4

### Synthesis, Purification and Characterization of O-acetyl-5050-PLGA

A 2000-mL, round-bottom flask equipped with an overhead stirrer was charged with purified 5050 PLGA [220 g, Mn of 5700] and DCM (660 mL). The mixture was stirred for 10 min to form a clear solution. Ac2O (11.0 mL, 116 mmol) and pyridine (9.4 mL, 116 mmol) were added to the solution, resulting in a minor exotherm of ˜0.5° C. The reaction was stirred at ambient temperature for 3 h and concentrated to ˜600 mL. The solution was added to a suspension of Celite® (660 g) in MTBE (6.6 L, 30 vol.) over 1 h with overhead stirring at ˜200 rpm. The suspension was filtered through a polypropylene filter and the filter cake was air-dried at ambient temperature for 1 day. It was suspended in acetone (1.6 L, ˜8 vol) with overhead stirring for 1 h. The slurry was filtered though a fritted funnel (coarse) and the filter cake was washed with acetone (3×300 mL). The combined filtrates were clarified though a Celite pad to afford a clear solution. It was concentrated to ˜700 mL and added to cold water (7.0 L, 0-5° C.) with overhead stirring at 200 rpm over 2 h. The suspension was filtered though a polypropylene filter. The filter cake was washed with water (3×500 mL), and conditioned for 1 h to afford 543 g of wet cake. It was transferred to two glass trays and air-dried at ambient temperature overnight to afford 338 g of wet product, which was then vacuum-dried at 25° C. for 2 days to constant weight to afford 201 g of product as a white powder [yield: 91%]. The ¹H NMR analysis was consistent with that of the desired product. The product was analyzed by HPLC (AUC, 230 nm) and GPC (Mw: 9.0 kDa and Mn: 6.3 kDa).

### Example 5

### Synthesis of PLGA-PEG-PLGA

The triblock copolymer PLGA-PEG-PLGA will be synthesized using a method developed by Zentner et al., Journal of Controlled Release, 72, 2001, 203-215. The molecular weight of PLGA obtained using this method would be ˜3 kDa. A similar method reported by Chen et al., International Journal of Pharmaceutics, 288, 2005, 207-218 will be used to synthesize PLGA molecular weights ranging from 1-7 kDa. The LA/GA ratio would typically be, but not limited to a ratio of 1:1. The minimum PEG molecular weight would be 2 kDa with an upper limit of 30 kDa. The preferred range of PEG would be 3-12 kDa. The PLGA molecular weight would be a minimum value of 4 kDa and a maximum of 30 kDa. The preferred range of PLGA would be 7-20 kDa. Any drug (e.g. any epothilone) could be conjugated to the PLGA through an appropriate linker (i.e. as listed in the previous examples) to form a polymer-drug conjugate. In addition, the same drug or a different drug could be attached to the other PLGA to form a dual drug polymer conjugate with two same drugs or two different drugs. Nanoparticles could be formed from either the PLGA-PEG-PLGA alone or from a single drug or dual polymer conjugate composed of this triblock copolymer.

### Example 6

### Synthesis of polycaprolactone-poly(ethylene glycol)-polycaprolactone (PCL-PEG-PCL)

The triblock PCL-PEG-PCL will be synthesized using a ring open polymerization method in the presence of a catalyst (i.e. stannous octoate) as reported in Hu et al., Journal of Controlled Release, 118, 2007, 7-17. The molecular weights of PCL obtained from this synthesis range from 2 to 22 kDa. A non-catalyst method shown in the article by Ge et al. Journal of Pharmaceutical Sciences, 91, 2002, 1463-1473 will also be used to synthesize PCL-PEG-PCL. The molecular weights of PCL that could be obtained from this particular synthesis range from 9 to 48 kDa. Similarly, another catalyst free method developed by Cerrai et al., Polymer, 30, 1989, 338-343 will be used to synthesize the triblock copolymer with molecular weights of PCL ranging from 1-9 kDa. The minimum PEG molecular weight would be 2 kDa with an upper limit of 30 kDa. The preferred range of PEG would be 3-12 kDa. The PCL molecular weight would be a minimum value of 4 kDa and a maximum of 30 kDa. The preferred range of PCL would be 7-20 kDa. Any drug (e.g., any epothilone) could be conjugated to the PCL through an appropriate linker (i.e. as listed in the previous examples) to form a polymer-drug conjugate. In addition, the same drug or a different drug could be attached to the other PCL to form a dual drug polymer conjugate with two same drugs or two different drugs. Nanoparticles could be formed from either the PCL-PEG-PCL alone or from a single drug or dual polymer conjugate composed of this triblock copolymer.

### Example 7

### Synthesis of polylactide-poly(ethylene glycol)-polylactide (PLA-PEG-PLA)

The triblock PLA-PEG-PLA copolymer will be synthesized using a ring opening polymerization using a catalyst (i.e. stannous octoate) reported in Chen et al., Polymers for Advanced Technologies, 14, 2003, 245-253. The molecular weights of PLA that can be formed range from 6 to 46 kDa. A lower molecular weight range (i.e. 1-8 kDa) could be achieved by using the method shown by Zhu et al., Journal of Applied Polymer Science, 39, 1990, 1-9. The minimum PEG molecular weight would be 2 kDa with an upper limit of 30 kDa. The preferred range of PEG would be 3-12 kDa. The PCL molecular weight would be a minimum value of 4 kDa and a maximum of 30 kDa. The preferred range of PCL would be 7-20 kDa. Any drug (e.g., any epothilone) could be conjugated to the PLA through an appropriate linker (i.e. as listed in the previous examples) to form a polymer-drug conjugate. In addition, the same drug or a different drug could be attached to the other PLA to form a dual drug polymer conjugate with two same drugs or two different drugs. Nanoparticles could be formed from either the PLA-PEG-PLA alone or from a single drug or dual polymer conjugate composed of this triblock copolymer.

### Example 8

### Synthesis of p-dioxanone-co-lactide-poly(ethylene glycol)-p-dioxanone-co-lactide (PDO-PEG-PDO)

The triblock PDO-PEG-PDO will be synthesized in the presence of a catalyst (stannous 2-ethylhexanoate) using a method developed by Bhattari et al., Polymer International, 52, 2003, 6-14. The molecular weight of PDO obtained from this method ranges from 2-19 kDa. The minimum PEG molecular weight would be 2 kDa with an upper limit of 30 kDa. The preferred range of PEG would be 3-12 kDa. The PDO molecular weight would be a minimum value of 4 kDa and a maximum of 30 kDa. The preferred range of PDO would be 7-20 kDa. Any drug (e.g., any epothilone) could be conjugated to the PDO through an appropriate linker (i.e. as listed in the previous examples) to form a polymer-drug conjugate. In addition, the same drug or a different drug could be attached to the other PDO to form a dual drug polymer conjugate with two same drugs or two different drugs. Nanoparticles could be formed from either the PDO-PEG-PDO alone or from a single drug or dual polymer conjugate composed of this triblock copolymer.

### Example 9

### Synthesis of Polyfunctionalized PLGA/PLA Based Polymers

One could synthesize a PLGA/PLA related polymer with functional groups that are dispersed throughout the polymer chain that is readily biodegradable and whose components are all bioacceptable components (i.e. known to be safe in humans). Specifically, PLGA/PLA related polymers derived from 3-S-[benzyloxycarbonyl)methyl]-1,4-dioxane-2,5-dione (BMD) could be synthesized (see structures below). (The structures below are intended to represent random copolymers of the monomeric units shown in brackets.)

1. PLGA/PLA related polymer derived from BMD

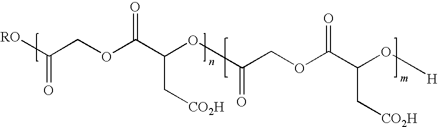

2. PLGA/PLA related polymer with BMD and 3,5-dimethyl-1,4-dioxane-2,5-dione (bis-DL-lactic acid cyclic diester)

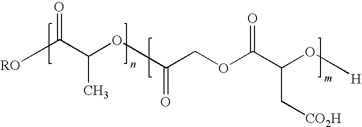

3. PLGA/PLA related polymer with BMD and 1,4-dioxane-2,5-dione (bis-glycolic acid cyclic diester

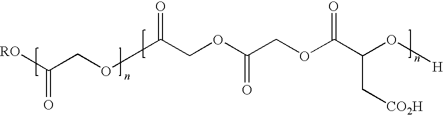

In a preferred embodiment, PLGA/PLA polymers derived from BMD and bis-DL-lactic acid cyclic diester will be prepared with a number of different pendent functional groups by varying the ratio of BMD and lactide. For reference, if it is assumed that each polymer has a number average molecular weight (Mn) of 8 kDa, then a polymer that is 100 wt % derived from BMD has approximately 46 pendant carboxylic acid groups (1 acid group per 0.174 kDa). Similarly, a polymer that is 25 wt % derived from BMD and 75 wt % derived from 3,5-dimethyl-1,4-dioxane-2,5-dione (bis-DL-lactic acid cyclic diester) has approximately 11 pendant carboxylic acid groups (1 acid group per 0.35 kDa). This compares to just 1 acid group for an 8 kDa PLGA polymer that is not functionalized and 1 acid group/2 kDa if there are 4 sites added during functionalization of the terminal groups of a linear PLGA/PLA polymer or 1 acid group/1 kDa if a 4 kDa molecule has four functional groups attached.

Specifically, the PLGA/PLA related polymers derived from BMD will be developed using a method by Kimura et al., Macromolecules, 21, 1988, 3338-3340. This polymer would have repeating units of glycolic and malic acid with a pendant carboxylic acid group on each unit [RO(COCH₂OCOCHR₁O)ₙ H where R is H, or alkyl or PEG unit etc. and R₁ is CO₂H]. There is one pendant carboxylic acid group for each 174 mass units. The molecular weight of the polymer and the polymer polydispersity can vary with different reaction conditions (i.e. type of initiator, temperature, processing condition). The Mn could range from 2 to 21 kDa. Also, there will be a pendant carboxylic acid group for every two monomer components in the polymer. Based on the reference previously sited, NMR analysis showed no detectable amount of the β-malate polymer was produced by ester exchange or other mechanisms.

Another type of PLGA/PLA related polymer derived from BMD and 3,5-dimethyl-1,4-dioxane-2,5-dione (bis-DL-lactic acid cyclic diester) will be synthesized using a method developed by Kimura et al., Polymer, 1993, 34, 1741-1748. They showed that the highest BMD ratio utilized was 15 mol % and this translated into a polymer containing 14 mol % (16.7 wt %) of BMD-derived units. This level of BMD incorporation represents approximately 8 carboxylic acid residues per 8 kDa polymer (1 carboxylic acid residue/kDa of polymer). Similarly to the use of BMD alone, no (3-malate derived polymer was detected. Also, Kimura et al. reported that the glass transition temperatures (T_{g}) were in the low 20° C.' s despite the use of high polymer molecular weights (36-67 kDa). The T_{g}'s were in the 20-23° C. for these polymers whether the carboxylic acid was free or still a benzyl group. The inclusion of more rigidifying elements (i.e. carboxylic acids which can form strong hydrogen bonds) should increase the T_{g}. Possible prevention of aggregation of any nanoparticles formed from a polymer drug conjugate derived from this specific polymer will have to be evaluated due to possible lower T_{g} values.

Another method for synthesizing a PLA-PEG polymer that contains varying amounts of glycolic acid malic acid benzyl ester involves the polymerization of BMD in the presence of 3,5-dimethyl-1,4-dioxane-2,5-dione (bis-DL-lactic acid cyclic diester), reported by Lee et al., Journal of Controlled Release, 94, 2004, 323-335. They reported that the synthesized polymers contained 1.3-3.7 carboxylic acid units in a PLA chain of approximately 5-8 kDa (total polymer weight was approximately 11-13 kDa with PEG being 5 kDa) depending on the quantity of BMD used in the polymerization. In one polymer there were 3.7 carboxylic acid units/hydrophobic block in which the BMD represents approximately 19 wt % of the weight of the hydrophobic block. The ratio of BMD to lactide was similar to that observed by Kimura et al., Polymer, 1993, 34, 1741-1748 and the acid residues were similar in the resulting polymers (approximately 1 acid unit/kDa of hydrophobic polymer).

Polymers functionalized with BMD that are more readily hydrolysable will be prepared using the method developed by Kimura et al., International Journal of Biological Macromolecules, 25, 1999, 265-271. They reported that the rate of hydrolysis was related to the number of free acid groups present (with polymers with more acid groups hydrolyzing faster). The polymers had approximately 5 or 10 mol % BMD content. Also, in the reference by Lee et al., Journal of Controlled Release, 94, 2004, 323-335, the rate of hydrolysis of the polymer was fastest with the highest concentration of pendent acid groups (6 days for polymer containing 19.5 wt % of BMD and 20 days for polymer containing 0 wt % of BMD.

A drug (e.g. an epothilone) could be conjugated to a PLGA/PLA related polymer with BMD (refer to previous examples above). Similarly, a nanoparticle could be prepared from such a polymer drug conjugate.

### Example 10

### Synthesis of Polymers Prepared Using β-Lactone of Malic Acid Benzyl Esters

One could prepare a polymer by polymerizing MePEGOH with RS-β-benzyl malolactonate (a β-lactone) with DL-lactide (cyclic diester of lactic acid) to afford a polymer containing MePEG (lactic acid) (malic acid) Me(OCH2CH2O)[OCCCH(CH3)O]m[COCH2CH(CO2H)O]. as developed by Wang et al., Colloid Polymer Sci., 2006, 285, 273-281. These polymers would potentially degrade faster because they contain higher levels of acidic groups. It should be noted that the use of β-lactones generate a different polymer from that obtained using 3-[benzyloxycarbonyl)methyl]-1,4-dioxane-2,5-dione. In these polymers, the carboxylic acid group is directly attached to the polymer chain without a methylene spacer.

Another polymer that could be prepared directly from a β-lactone was reported by Ouhib et al., Ch. Des. Monoeres. Polym, 2005, 1, 25. The resulting polymer (i.e. poly-3,3-dimethylmaleic acid) is water soluble as the free acid, has pendant carboxylic acid groups on each unit of the polymer chain and as well it has been reported that 3,3-dimethylmaleic acid is a nontoxic molecule.

One could polymerize 4-benzyloxycarbonyl-,3,3-dimethyl-2-oxetanone in the presence of 3,5-dimethyl-1,4-dioxane-2,5-dione (DDD) and β-butyrolactone to generate a block copolymer with pendant carboxylic acid groups as shown by Coulembier et al., Macromolecules, 2006, 39, 4001-4008. This polymerization reaction was carried out with a carbene catalyst in the presence of ethylene glycol. The catalyst used was a triazole carbene catalyst which leads to polymers with narrow polydispersities.

What is claimed is:

1. (canceled)

2. A particle, comprising:
a) a plurality of hydrophobic polymer-epothilone conjugates, wherein
i) each hydrophobic polymer-epothilone conjugate of said plurality comprises a hydrophobic polymer attached to epothilone,
ii) said hydrophobic polymer attached to epothilone can be a homopolymer or a polymer made up of more than one kind of monomeric subunit,
iii) said hydrophobic polymer attached to said epothilone has a weight average molecular weight of about 4-15 kD,
iv) said epothilone is about 1-30 weight % of said particle and
v) said plurality of hydrophobic polymer-epothilone conjugates is about 25-80 weight % of said particle;
b) a plurality of hydrophilic-hydrophobic polymers, wherein
i) each of said hydrophilic-hydrophobic polymers of said plurality comprises a hydrophilic portion attached to a hydrophobic portion,
ii) said hydrophilic portion has a weight average molecular weight of about 1-6 kD (e.g., 2-6 kD), and
iii) said plurality of hydrophilic-hydrophobic polymers is about 5-30 weight % of said particle;
and
c) a surfactant, wherein said surfactant is about 15-35 weight % of said particle; and
wherein:
the diameter of said particle is less than about 200 nm.

3. The particle of claim 2, wherein a)iii) said hydrophobic polymer attached to said epothilone has a weight average molecular weight of about 4-8 kD.

4. The particle of claim 2, wherein a)iii) said hydrophobic polymer attached to said epothilone has a weight average molecular weight of about 8-13 kD.

5. The particle of claim 2,
wherein
if the weight average molecular weight of said hydrophilic portion of said hydrophilic-hydrophobic polymer is about 1-3 kD, e.g., about 2 kD, the ratio of the weight average molecular weight of said hydrophilic portion to the weight average molecular weight of said hydrophobic portion is between 1:3-1:7, and if the weight average molecular weight of said hydrophilic portion is about 4-6 kD, e.g., about 5 kD, the ratio of the weight average molecular weight of said hydrophilic portion to the weight average molecular weight of said hydrophobic portion is between 1:1-1:4.

6-7. (canceled)

8. The particle of claim 2, wherein
said hydrophilic portion of said hydrophilic-hydrophobic polymer terminates in an OMe,
and
said particle further comprises a hydrophobic polymer having a terminal acyl moiety.

9-10. (canceled)

11. A method of making the particle of claim 2, comprising:
providing an organic solution comprising:
a) a plurality of hydrophobic polymer-epothilone conjugates, wherein
i) each hydrophobic polymer-epothilone conjugate of said plurality comprises a hydrophobic polymer attached to an epothilone,
ii) said hydrophobic polymer attached to said epothilone can be a homopolymer or a polymer made up of more than one kind of monomeric subunit,
iii) said hydrophobic polymer attached to said epothilone has a weight average molecular weight of about 4-15 kD,
iv) said epothilone is about 1-30 weight % of said particle and
v) said plurality of hydrophobic polymer-epothilone conjugates is about 25-80 weight % of said particle;
b) a plurality of hydrophilic-hydrophobic polymers, wherein
i) each of said hydrophilic-hydrophobic polymers of said plurality comprises a hydrophilic portion attached to a hydrophobic portion,
ii) said hydrophilic portion has a weight average molecular weight of about 1-6 kD (e.g., 2-6 kD), and
iii) said plurality of hydrophilic-hydrophobic polymers is about 5-30 weight % of said particle;
and
combining said organic solution with an aqueous solution comprising a solvent to provide said particles.

12-13. (canceled)

14. A pharmaceutically acceptable composition comprising a plurality of particles of claim 2 and an additional component.

15. A kit comprising a plurality of particles of claim 2.

16. A single dosage unit comprising a plurality of particles of claim 2.

17. A method of treating a subject having a disorder comprising administering to said subject an effective amount of particles of claim 2.

18. The particle of claim 2 comprising:
b) a plurality of PEG-hydrophobic polymers, wherein
i) each of said PEG-hydrophobic polymers of said plurality comprises a PEG portion attached to a hydrophobic portion,
ii) said PEG portion has a weight average molecular weight of about 1-6 kD (e.g., 2-6 kD), and
iii) said plurality of PEG-hydrophobic polymers is about 5-30 weight % of said particle;
and
c) PVA, wherein
said PVA has a weight average molecular weight of about 5-45 kD and is about 15-35 weight of said particle.

19-20. (canceled)

21. The particle of claim 18,
wherein
if the weight average molecular weight of said PEG portion of said PEG-hydrophobic polymer is about 1-3 kD, e.g., about 2 kD, the ratio of the weight average molecular weight of said PEG portion to the weight average molecular weight of said hydrophobic portion is between 1:3-1:7, and if the weight average molecular weight of said PEG portion is about 4-6 kD, e.g., about 5 kD, the ratio of the weight average molecular weight of said PEG portion to the weight average molecular weight of said hydrophobic portion is between 1:1-1:4; and
iii) said plurality of PEG-hydrophobic polymers is about 5-30 weight % of said particle.

22-23. (canceled)

24. The particle of claim 18, wherein
the hydrophobic polymer is made up of a first and a second type of monomeric subunit, and the ratio of the first to second type of monomeric subunit in said hydrophobic polymer attached to said epothilone is from about 25:75 to about 75:25, and wherein
said PEG portion of said PEG-hydrophobic polymer terminates in an OMe,
and
wherein said PVA has a weight average molecular weight of about 23-26 kD;
wherein:
the particle further comprises a hydrophobic polymer having a terminal acyl moiety.

25-26. (canceled)

27. A method of making the particle of claim 18, comprising:
providing an organic solution comprising:
a) a plurality of hydrophobic polymer-epothilone conjugates, wherein
i) each hydrophobic polymer-epothilone conjugate of said plurality comprises a hydrophobic polymer attached to an epothilone,
ii) the hydrophobic polymer is made up of a first and a second type of monomeric subunit, and the ratio of the first to second type of monomeric subunit in said hydrophobic polymer attached to said epothilone is from about 25:75 to about 75:25,
iii) said hydrophobic polymer attached to said epothilone has a weight average molecular weight of about 4-15 kD,
iv) said epothilone is about 1-30 weight % of said particle and
v) said plurality of hydrophobic polymer-epothilone conjugates is about 25-80 weight % of said particle;
b) a plurality of PEG-hydrophobic polymers, wherein
i) each of said PEG-hydrophobic polymers of said plurality comprises a PEG portion attached to a hydrophobic portion,
ii) said PEG portion has a weight average molecular weight of about 1-6 kD (e.g., 2-6 kD), and
iii) said plurality of PEG-hydrophobic polymers is about 5-30 weight % of said particle; and
combining the organic solution with an aqueous solution comprising PVA to provide said particles.

28-33. (canceled)

34. The particle of claim 2 comprising:
a) a plurality of PLGA-epothilone (e.g., therapeutic or diagnostic epothilone) conjugates, wherein
i) each PLGA-epothilone conjugate of said plurality comprises a PLGA polymer attached to an epothilone,
ii) the ratio of lactic acid to glycolic acid in said PLGA polymer attached to said epothilone is from about 25:75 to about 75:25,
iii) said PLGA polymer attached to said epothilone has a weight average molecular weight of about 4-15 kD,
iv) said epothilone is about 1-30 weight % of said particle and
v) said plurality of PLGA-epothilone conjugates is about 25-80 weight % of said particle;
b) a plurality of PEG-PLGA polymers, wherein
i) each of said PEG-PLGA polymers of said plurality comprises a PEG portion attached to a PLGA portion,
ii) said PEG portion has a weight average molecular weight of about 1-6 kD (e.g., 2-6 kD), and
iii) said plurality of PEG-PLGA polymers is about 5-30 weight % of said particle;
and
c) PVA, wherein
said PVA has a weight average molecular weight of about 5-45 kD and is about 15-35 weight % of said particle; and
wherein:
the diameter of said particle is less than about 200 nm.

35-36. (canceled)

37. The particle of claim 34, wherein
if the weight average molecular weight of said PEG portion of said PEG-PLGA polymer is about 1-3 kD, e.g., about 2 kD, the ratio of the weight average molecular weight of said PEG portion to the weight average molecular weight of said PLGA portion is between 1:3-1:7, and if the weight average molecular weight of said PEG portion is about 4-6 kD, e.g., about 5 kD, the ratio of the weight average molecular weight of said PEG portion to the weight average molecular weight of said PLGA portion is between 1:1-1:4.

38-39. (canceled)

40. The particle of claim 34,
wherein
said PEG portion of said PEG-PLGA polymer has a weight average molecular weight of about 2-6 kD and said PLGA portion has a weight average molecular weight of between about 8-13 kD,
iii) said plurality of PEG-PLGA polymers is about 10-25 weight % of said particle;
iv) said PEG portion of said PEG-PLGA polymer terminates in an OMe,
and
wherein said PVA has a weight average molecular weight of about 23-26 kD and is about 15-35 weight % of said particle; and
wherein:
said particle further comprises PLGA having a terminal acyl moiety.

41-49. (canceled)

50. The particle of claim 2, wherein said epothilone has the structure of Formula XI: 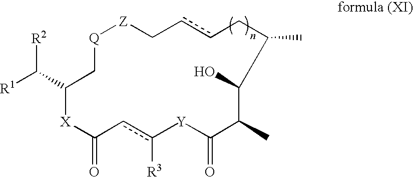
wherein
R¹ is aryl, heteroaryl, arylalkenyl, or heteroarylalkenyl; each of which is optionally substituted with 1-3 R⁸;
R² is H or alkyl (e.g., methyl); or
R¹ and R², when taken together with the carbon to which they are attached, form an aryl or a heteroaryl moiety optionally substituted with 1-3 R⁸;
R³ is H, OH, NH₂ or CN;
X is O or NR⁴;
R⁴ is H, alkyl, —C(O)Oalkyl, —C(O)Oarylalkyl, —C(O)NR⁵alkyl, —C(O)NR⁵arylalkyl, —C(O)alkyl, —C(O)aryl or arylalkyl;
Y is CR⁵R⁶, O or NR⁷;
each of R⁵ and R⁶ is independently H or alkyl (e.g., methyl);
R⁷ is H, alkyl, —C(O)Oalkyl, —C(O)Oarylalkyl, —C(O)NR⁵alkyl, —C(O)NR⁵arylalkyl, —C(O)alkyl, —C(O)aryl or arylalkyl;
each R⁸, for each occurrence, is independently alkyl, aminoalkyl, hydroxyalkyl, alkylthiol, aryl, arylalkyloxyalkyl or alkoxy;
Q-Z, when taken together, form 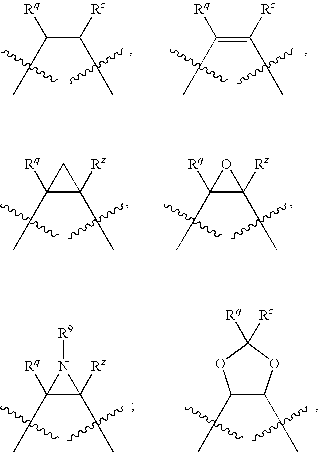
heteroarylenyl, C(O)NR⁴, NR⁴C(O), CR⁵R⁶NR⁴, or
NR⁴CR⁵R⁶NR⁴;
R^{q} is H, alkyl (e.g., methyl) or hydroxy;
R^{z} is H, alkyl (e.g., methyl), haloalkyl (e.g., CF₃), heterocyclylalkyl or N₃;
R⁹ is H, alkyl, —C(O)Oalkyl, —C(O)Oarylalkyl, —C(O)NR⁵alkyl, —C(O)NR⁵arylalkyl, —C(O)alkyl, —C(O)aryl or arylalkyl;
each [symbol] for each occurrence, is independently a single or double bond; and
n is 0, 1 or 2.

51. The particle of claim 2, wherein said epothilone has the structure of Formula XId: 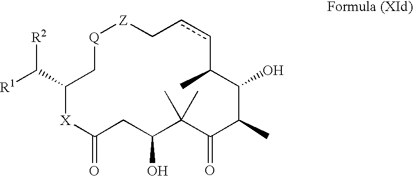
wherein
R¹ is heteroarylalkenyl, which is optionally substituted with 1-3 R⁸;
R² is alkyl (e.g., methyl); or
R¹ and R², when taken together with the carbon to which they are attached, form a heteroaryl moiety substituted with 1 R⁸;
X is O or NR⁴;
R⁴ is H;
Y is CR⁵R⁶;
each of R⁵ and R⁶ is independently alkyl (e.g., methyl);
R⁸ is alkyl (e.g., methyl);
Q-Z, when taken together, form 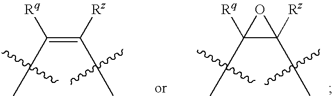
R^{q} is H or alkyl (e.g., methyl);
R^{z} is H or alkyl (e.g., methyl); and [symbol]
is a single or double bond.

52. The particle of claim 2, wherein said epothilone is epothilone B.

53. The particle of claim 2, wherein said epothilone is ixabepilone.

54. The particle claim 2, wherein said epothilone is BMS-310705.

55. The particle of claim 2, wherein said epothilone is epothilone D.

56. The particle of claim 2, wherein said epothilone is dehydelone.

57. The particle of claim 2, wherein said epothilone is sagopilone (ZK-EPO).

58-105. (canceled)

106. A polymer-agent conjugate, wherein said agent is an epothilone, comprising:
a hydrophobic polymer; and
an epothilone attached to said polymer.

107-115. (canceled)

116. The polymer-agent conjugate of claim 106, having the formula: 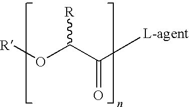
wherein:
agent is an epothilone
L is selected from a bond or linker;
R is selected from hydrogen and methyl, wherein about 45% to about 55% of R substituents are hydrogen and about 45% to about 55% are methyl;
R′ is selected from hydrogen, acyl and a hydroxy protecting group; and
n is an integer from about 15 to about 308.

117-129. (canceled)

130. The particle of claim 2, wherein a)iii) said hydrophobic polymer attached to said epothilone has a weight average molecular weight of about 9-12 kD.

131-141. (canceled)
